(19) United States
(12) Patent Application Publication
(10) Pub. No.: US 2025/0391505 A1
(43) Pub. Date: Dec. 25, 2025

(54) Methods and Systems for Machine Learning Analysis of Lupus Nephritis

(71) Applicant: AMPEL BioSolutions, LLC, Charlottesville, VA (US)

(72) Inventors: Kathryn K. ALLISON, Charlottesville, VA (US); Sneha SHROTRI, Charlottesville, VA (US); Andrea DAAMEN, Charlottesville, VA (US); Amrie C. GRAMMER, Charlottesville, VA (US); Peter E. LIPSKY, Charlottesville, VA (US)

(21) Appl. No.: 19/020,679

(22) Filed: Jan. 14, 2025

Related U.S. Application Data
(63) Continuation of application No. PCT/US2023/027847, filed on Jul. 14, 2023.
(60) Provisional application No. 63/389,804, filed on Jul. 15, 2022.
(60) Provisional application No. 63/424,096, filed on Nov. 9, 2022.
(60) Provisional application No. 63/448,628, filed on Feb. 27, 2023.

(51) Int. Cl. G16B 25/10; G16B 40/00; G16H 10/60; G16H 50/20

(52) U.S. Cl. CPC G16B 25/10; G16B 40/00; G16H 10/60; G16H 50/20

(57) ABSTRACT

A method for assessing a lupus nephritis disease state of a patient, the method comprising: analyzing a data set comprising or derived from gene expression measurement data of at least 2 genes or human orthologs thereof selected from the genes listed in Tables 19-1 to 19-36, Tables 19A-1 to 19A-36, Table 20, Table 21, Table 22, Tables 23-1 to 23-28, Tables 25-1 to 25-32, Tables 26-1 to 26-60, Tables 27-1 to 27-48, and Tables 28-1 to 28-22 in a biological sample from the patient, to classify the lupus nephritis disease state of the patient.

This application is a continuation of PCT Application No. PCT/US2023/027847, filed Jul. 14, 2023, which claims priority to U.S. Provisional Patent Application No. 63/389,804, filed Jul. 15, 2022; U.S. Provisional Patent Application No. 63/424,096, filed Nov. 9, 2022; and U.S. Provisional Patent Application No. 63/448,628, filed Feb. 27, 2023, all of which are incorporated in full herein by reference.

### BACKGROUND

Systemic lupus erythematosus (SLE) is an autoimmune disorder that can affect a variety of tissues, including the kidney. Lupus nephritis (LN) is one of the most severe organ manifestations of SLE and affects approximately 40% of adult lupus patients with 10-20% of patients developing end-stage renal disease (ESRD). The immune mechanisms of LN disease progression and risk factors for end organ damage are poorly understood. There is a need for understanding molecular pathways involved in disease progression in LN to allow identification and optimization of therapies.

### SUMMARY

An aspect of the current disclosure is directed to a method for assessing a lupus nephritis (LN) disease state of a patient. Based on transcriptomic analysis of lupus prone mice, the inventors have identified molecular pathways and risk factors for development of end-stage renal disease in human lupus patients. Using a gene expression-based clustering approach, disclosed sets of curated gene signatures are identified which, can be used e to classify disease stages of murine glomerulonephritis into molecular endotypes that effectively translate to human LN patients. A newly recognized, intermediate stage (e.g., endotype) of LN, referred to herein as “transitional LN”, occurring between acute and chronic LN disease state, was identified. Based on an understanding of molecular mechanisms of LN disease state progression from acute LN disease state to transitional LN disease state, and transitional LN disease state to chronic LN disease state, and gene expression analysis of the molecular endotypes (e.g., acute LN, transitional LN and chronic LN), targeted therapy was developed to stop, slow and/or reverse LN disease progression in a patient. The method for assessing the LN disease state of the patient can include analyzing a data set comprising or derived from gene expression measurement data of at least 2 genes or human orthologs thereof, from a biological sample from the patient, to classify the LN disease state of the patient. In certain embodiments, the at least 2 genes are selected from the genes listed in Tables 19-1 to 19-36, Tables 19A-1 to 19A-36, Table 20, Table 21, Table 22, Tables 23-1 to 23-28, Tables 25-1 to 25-32, Tables 26-1 to 26-60, Tables 27-1 to 27-48 and Tables 28-1 to 28-22. As an illustrative example, “genes listed in Table X and Y” includes x+y genes, where Table X contains x genes and Table Y contains y genes, considering no overlap exists between x and y genes. In the event of overlap, duplicate copies can be excluded from analysis.

In certain embodiments, the data set comprises or is derived from gene expression measurement data of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 450, 500, 550, 600, 650, 700, 750, 850, 900, 950, 1000, 1050, 1100, 1150, 1200, 1250, 1300, 1350, 1400, 1450, 1500, 1550, 1600, 1700, 1800, 1900, 2000 or all, or any range or value therebetween, genes (or human orthologs thereof), selected from the genes listed in Tables 19-1 to 19-36, Tables 19A-1 to 19A-36, Table 20, Table 21, Table 22, Tables 23-1 to 23-28, Tables 25-1 to 25-32, Tables 26-1 to 26-60, Tables 27-1 to 27-48, and Tables 22-1 to 22-28, from the biological sample from the patient. In certain embodiments, the at least two genes are selected from the genes listed in Tables 19-1 to 19-36. In certain embodiments, the at least two genes are selected from the genes listed in Tables 19A-1 to 19A-36. In certain embodiments, the at least two genes are selected from the genes listed in Table 20. In certain embodiments, the at least two genes are selected from the genes listed in Table 21. In certain embodiments, the at least two genes are selected from the genes listed in Table 22. In certain embodiments, the at least two genes are selected from the genes listed in Tables 23-1 to 23-28. In certain embodiments, the at least two genes are selected from the genes listed in Tables 25-1 to 25-32. In certain embodiments, the at least two genes are selected from the genes listed in Tables 26-1 to 26-60. In certain embodiments, the at least two genes are selected from the genes listed in Tables 27-1 to 27-48. In certain embodiments, the at least two genes are selected from the genes listed in Tables 28-1 to 28-22. The at least 2 genes may or may not include gene(s) that are not listed in Tables 19-1 to 19-36, Table 20, Table 21, Table 22, Tables 23-1 to 23-28, Tables 25-1 to 25-32, Tables 26-1 to 26-60, Tables 27-1 to 27-48, and/or Tables 28-1 to 28-22. In certain embodiments, the at least 2 genes do not include any gene that are not listed in Tables 19-1 to 19-36, Table 20, Table 21, Table 22, Tables 23-1 to 23-28, Tables 25-1 to 25-32, Tables 26-1 to 26-60, Tables 27-1 to 27-48, and/or Tables 28-1 to 28-22. In certain embodiments, the at least 2 genes do not include any gene that is not listed in Tables 19-1 to 19-36. In certain embodiments, the at least 2 genes do not include any gene that is not listed in Tables 23-1 to 23-28. In certain embodiments, the at least 2 genes do not include any gene that is not listed in Tables 25-1 to 25-32. In certain embodiments, the at least 2 genes do not include any gene that is not listed in Tables 26-1 to 26-60. In certain embodiments, the at least 2 genes do not include any gene that is not listed in Tables 27-1 to 27-48. In certain embodiments, the at least 2 genes do not include any gene that is not listed in Tables 28-1 to 28-22. In certain embodiments, the data set comprises or is derived from gene expression measurement data of one or more human orthologs of the genes selected from Tables 19-1 to 19-36, Table 20, Table 21, Table 22, Tables 28-1 to 28-22, Tables 26-1 to 26-60, and Tables 27-1 to 27-48. Gene sets listed in each of these Tables can be used as effective biomarkers for classifying the LN disease state of the patients. In certain embodiments, the data set comprises or is derived from gene expression measurement data of one or more human orthologs of the genes selected from Tables 19-1 to 19-36. In certain embodiments, the data set comprises or is derived from gene expression measurement data of one or more human orthologs of the genes selected from Table 20. In certain embodiments, the data set comprises or is derived from gene expression measurement data of one or more human orthologs of the genes selected from Table 21. In certain embodiments, the data set comprises or is derived from gene expression measurement data of one or more human orthologs of the genes selected from Table 22. A human ortholog of a non-human gene (such as a mouse gene) can be identified using a method as described in U.S. Pat. App. Pub. No. 2021/0104321 (“Machine Learning Disease Prediction and Treatment Prioritization”), incorporated herein by reference in its entirety, as described in the Examples therein, and/or by any method published and/or known to one of skill in the art. As a non-limiting example, human orthologs of the mouse gene sets can be identified on a gene-by-gene basis using publicly available online databases, including but not limited to GeneCards, the Mouse Genome Informatics (MGI), and UniProtKB, as well as literature mining. Through this process, genes with similar tissue expression, cellular localization, and functions between mouse and human can be retained in the human gene sets. One or more human ortholog of a non-human gene may be identified. Gene expression measurement data of any of the one or more identified human orthologs of a given non human gene may be comprised by the data set. It is understood that in the absence of a human ortholog for a given non human gene, that expression measurement data of human ortholog for that non human gene may not be comprised by the data set.

In certain embodiments, the data set comprises or is derived from gene expression measurement data of human orthologs of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 450, 500, 550, 600, 650, 700, 750, 850, 900, 950, 1000, 1050, 1100, 1150, 1200, 1250, or 1291 or all, or any range or value therebetween, genes selected from the genes listed in Tables 19-1 to 19-36, from the biological sample from the patient.

In certain embodiments, the data set comprises or is derived from gene expression measurement data of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 450, 500, 550, 600, 650, 700, 750, 850, 900, 950, 1000, 1050, 1100, 1150, 1200, 1250, or 1291 or all, or any range or value therebetween, genes selected from the genes listed in Tables 19A-1 to 19A-36, from the biological sample from the patient.

In certain embodiments, the data set comprises or is derived from gene expression measurement data of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 450, 500, 550, 600, 650, 700, 750, 850, 900, 950, 1000, 1050, 1100, 1150, 1200, 1250, 1300, 1500, 2000, or all or any range or value there between genes, selected from the genes listed in Tables 26-1 to 26-60, from the biological sample from the patient.

In certain embodiments, the data set comprises or is derived from gene expression measurement data of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 450, 500, 550, 600, 650, 700, 750, 850, 900, 950, 1000, 1050, 1100, 1150, 1200, 1250, 1300, 1500, 2000, or all or any range or value there between genes, selected from the genes listed in Tables 27-1 to 27-48, from the biological sample from the patient.

In certain embodiments, the data set comprises or is derived from gene expression measurement data of human orthologs of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 450, 500, 550, 600, 650, 700, 727, or all, or any range or value there between genes, selected from the genes listed in Tables 28-1 to 28-22, from the biological sample from the patient.

In certain embodiments, the data set comprises or is derived from gene expression measurement data of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 450, 500, 550, 600, 650, 700, 750, 850, 900, 950, 960, 968, or all, or any range or value therebetween, genes selected from the genes listed in Tables 23-1 to 23-28, from the biological sample from the patient.

In certain embodiments, the data set comprises or is derived from gene expression measurement data of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 450, 500, 550, 600, 650, 700, 750, 850, 900, 950, 960, 968, 1000, or all, or any range or value therebetween, genes selected from the genes listed in Tables 25-1 to 25-32, from the biological sample from the patient.

In certain embodiments, the data set comprises or is derived from gene expression measurement data of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 450, 500, 550, 600, 650, 700, 727, or all, or any range or value there between genes, selected from the genes listed in Tables 28-1 to 28-22, from the biological sample from the patient.

In certain embodiments, the data set comprises or is derived from gene expression measurement data of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, or 203, or all, or any range or value there between, genes or human orthologs thereof selected from the genes listed in each of one or more Tables selected from Tables 19-1 to 19-36, Tables 19A-1 to 19A-36, Table 20, Table 21, Table 22, Tables 23-1 to 23-28, Tables 25-1 to 25-32, Tables 26-1 to 26-60, Tables 27-1 to 27-48, and Table 28-1 to 28-22 from the biological sample from the patient, wherein the number of genes selected from the genes listed in each selected table may be different or same (e.g., a different or identical number of genes can be selected from the genes listed in each selected table, e.g., in an illustrative example Table 25-1, Table 25-2, and Table 25-3 are selected, and 4 genes from Table 25-1, 2 genes from Table 25-2, and 7 genes from Table 25-3 are selected). In a non-limiting example, the data set comprises or is derived from gene expression measurement data of at least 2 genes selected from the genes listed in each of 28 tables (i.e., one or more Tables selected comprises 28 tables) selected from Tables 23-1 to 23-28, from the biological sample from the patient, i.e., 28 Tables from Tables 23-1 to 23-28 are selected, and at least 2 genes are selected from the genes listed in each of the selected Tables, thereby the data set comprises or is derived from gene expression measurement data of, at least 2 genes selected from the genes listed in Table 23-1, at least 2 genes selected from the genes listed in Table 23-2, at least 2 genes selected from the genes listed in Table 23-3, at least 2 genes selected from the genes listed in Table 23-4, at least 2 genes selected from the genes listed in Table 23-5, at least 2 genes selected from the genes listed in Table 23-6, at least 2 genes selected from the genes listed in Table 23-7, at least 2 genes selected from the genes listed in Table 23-8, at least 2 genes selected from the genes listed in Table 23-9, at least 2 genes selected from the genes listed in Table 23-10, at least 2 genes selected from the genes listed in Table 23-11, at least 2 genes selected from the genes listed in Table 23-12, at least 2 genes selected from the genes listed in Table 23-13, at least 2 genes selected from the genes listed in Table 23-14, at least 2 genes selected from the genes listed in Table 23-15, at least 2 genes selected from the genes listed in Table 23-16, at least 2 genes selected from the genes listed in Table 23-17, at least 2 genes selected from the genes listed in Table 23-18, at least 2 genes selected from the genes listed in Table 23-19, at least 2 genes selected from the genes listed in Table 23-20, at least 2 genes selected from the genes listed in Table 23-21, at least 2 genes selected from the genes listed in Table 23-22, at least 2 genes selected from the genes listed in Table 23-23, at least 2 genes selected from the genes listed in Table 23-24, at least 2 genes selected from the genes listed in Table 23-25, at least 2 genes selected from the genes listed in Table 23-26, at least 2 genes selected from the genes listed in Table 23-27, and at least 2 genes selected from the genes listed in Table 23-28, from the biological sample from the patient. Genes selected from each selected Table of the one or more Tables, can be used as effective biomarkers for classifying the LN disease state of the patient.

In certain embodiments, the data set comprises or is derived from gene expression measurement data of one or more human orthologs of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, or 191 or all, or any range or value there between, genes selected from the genes listed in each of one or more Tables selected from Tables 19-1 to 19-36, from the biological sample from the patient, wherein the number of genes selected from the genes listed in each selected table may be different or same (e.g., a different or identical number of genes can be selected from the genes listed in each selected table). In certain embodiments, the data set comprises or is derived from gene expression measurement data of one or more human orthologs of an effective number of genes selected from the genes listed in each of the one or more Tables selected from Tables 19-1 to 19-36, from the biological sample from the patient, wherein a different or identical number of genes can be selected from the genes listed in each selected table. In certain embodiments, the data set comprises or is derived from gene expression measurement data of one or more human orthologs of the genes listed in each of one or more Tables selected from Tables 19-1 to 19-36, from the biological sample from the patient. The one or more Tables selected from Tables 19-1 to 19-36 can include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36, or any range therebetween Tables. In certain embodiments, Tables 19-1 to 19-36 (e.g., 36 Tables) are selected.

In certain embodiments, the data set comprises or is derived from gene expression measurement data of one or more human orthologs of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, or 191 or all, or any range or value there between, genes selected from the genes listed in each of one or more Tables selected from Tables 19-1 to 19-36, from the biological sample from the patient, wherein the number of genes selected from the genes listed in each selected table may be different or same (e.g., a different or identical number of genes can be selected from the genes listed in each selected table). In certain embodiments, the data set comprises or is derived from gene expression measurement data of one or more human orthologs of an effective number of genes selected from the genes listed in each of the one or more Tables selected from Tables 19-1 to 19-36, from the biological sample from the patient, wherein a different or identical number of genes can be selected from the genes listed in each selected table. In certain embodiments, the data set comprises or is derived from gene expression measurement data of one or more human orthologs of the genes listed in each of one or more Tables selected from Tables 19-1 to 19-36, from the biological sample from the patient. The one or more Tables selected from Tables 19-1 to 19-36 can include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36, or any range therebetween Tables. In certain embodiments, Tables 19-1 to 19-36 (e.g., 36 Tables) are selected.

In certain embodiments, the data set comprises or is derived from gene expression measurement data of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, or 191 or all, or any range or value there between, genes selected from the genes listed in each of one or more Tables selected from Tables 19A-1 to 19A-36, from the biological sample from the patient, wherein the number of genes selected from the genes listed in each selected table may be different or same (e.g., a different or identical number of genes can be selected from the genes listed in each selected table). In certain embodiments, the data set comprises or is derived from gene expression measurement data of an effective number of genes selected from the genes listed in each of the one or more Tables selected from Tables 19A-1 to 19A-36, from the biological sample from the patient, wherein a different or identical number of genes can be selected from the genes listed in each selected table. In certain embodiments, the data set comprises or is derived from gene expression measurement data of the genes listed in each of one or more Tables selected from Tables 19A-1 to 19A-36, from the biological sample from the patient. The one or more Tables selected from Tables 19A-1 to 19A-36 can include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36, or any range therebetween Tables. In certain embodiments, Tables 19A-1 to 19A-36 (e.g., 36 Tables) are selected.

In certain embodiments, the data set comprises or is derived from gene expression measurement data of one or more human orthologs of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, or all, or any range or value there between, genes selected from the genes listed in each of one or more Tables selected from Tables 28-1 to 28-22, from the biological sample from the patient, wherein the number of genes selected from the genes listed in each selected table may be different or same (e.g., a different or identical number of genes can be selected from the genes listed in each selected table). In certain embodiments, the data set comprises or is derived from gene expression measurement data of one or more human orthologs of an effective number of genes selected from the genes listed in each of the one or more Tables selected from Tables 28-1 to 28-22, from the biological sample from the patient, wherein a different or identical number of genes can be selected from the genes listed in each selected table. In certain embodiments, the data set comprises or is derived from gene expression measurement data of one or more human orthologs of the genes listed in each of the one or more Tables selected from Tables 28-1 to 28-22, from the biological sample from the patient. The one or more Tables selected from Tables 28-1 to 28-22 can include at least 1, 2, 3, 4, 5, 6, 7, 8, 9, 10, 11, 12, 13, 14, 15, 16, 17, 18, 19, 20, 21, or 22, or any range there between Tables. In certain embodiments, Tables 28-1 to 28-22 (e.g., 22 Tables) are selected. In certain embodiments, Tables 28-1, 28-2, 28-3, 28-4, 28-5, 28-6, 28-7, 28-8, 28-12, 28-13, 28-14, 28-15, 28-16, 28-17, 28-18, 28-19, 28-20, 28-21 and 28-22 are selected. In certain embodiments, Tables 28-1, 28-2, 28-3, 28-4, 28-6, 28-7, 28-8, 28-9, 28-10, 28-11, 28-12, 28-13, 28-14, 28-15, 28-16, 28-17, 28-18, 28-20, 28-21 and 28-22 are selected. In certain embodiments, Tables 28-1, 28-2, 28-3, 28-4, 28-5, 28-6, 28-7, 28-8, 28-9, 28-10, 28-11, 28-12, 28-13, 28-14, 28-15, 28-16, 28-17, 28-18, 28-20, 28-21 and 28-22 are selected. In certain embodiments, Tables 28-1 to 28-22 (e.g., 22 Tables) are selected.

In certain embodiments, the data set comprises or is derived from gene expression measurement data of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, or 191 or all, or any range or value therebetween, genes selected from the genes listed in each of one or more Tables selected from Tables 26-1 to 26-60, from the biological sample from the patient, wherein the number of genes selected from the genes listed in each selected table may be different or same (e.g., a different or identical number of genes can be selected from the genes listed in each selected table). In certain embodiments, the data set comprises or is derived from gene expression measurement data of an effective number of genes selected from the genes listed in each of the one or more Tables selected from Tables 26-1 to 26-60, from the biological sample from the patient, wherein a different or identical number of genes can be selected from the genes listed in each selected table. In certain embodiments, the data set comprises or is derived from gene expression measurement data of the genes listed in each of the one or more Tables selected from Tables 26-1 to 26-60, from the biological sample from the patient. The one or more Tables selected from Tables 26-1 to 26-60 can include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60, or any range there between Tables. In certain embodiments, Tables 26-1 to 26-60 (e.g., 60 Tables) are selected.

In certain embodiments, the data set comprises or is derived from gene expression measurement data of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, or 191 or all, or any range or value therebetween, genes selected from the genes listed in each of one or more Tables selected from Tables 27-1 to 27-48, from the biological sample from the patient, wherein the number of genes selected from the genes listed in each selected table may be different or same (e.g., a different or identical number of genes can be selected from the genes listed in each selected table). In certain embodiments, the data set comprises or is derived from gene expression measurement data of an effective number of genes selected from the genes listed in each of the one or more Tables selected from Tables 27-1 to 27-48, from the biological sample from the patient, wherein a different or identical number of genes can be selected from the genes listed in each selected table. In certain embodiments, the data set comprises or is derived from gene expression measurement data of the genes listed in each of the one or more Tables selected from Tables 27-1 to 27-48, from the biological sample from the patient. The one or more Tables selected from Tables 27-1 to 27-48 can include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or any range there between Tables. In certain embodiments, Tables 27-1 to 27-48 (e.g., 48 Tables) are selected.

In certain embodiments, the data set comprises or is derived from gene expression measurement data of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, or 203, or all, or any range or value therebetween, genes selected from the genes listed in each of one or more Tables selected from Tables 23-1 to 23-28, from the biological sample from the patient, wherein the number of genes selected from the genes listed in each selected table may be different or same (e.g., a different or identical number of genes can be selected from the genes listed in each selected table). In certain embodiments, the data set comprises or is derived from gene expression measurement data of an effective number of genes selected from the genes listed in each of the one or more Tables selected from Tables 23-1 to 23-28, from the biological sample from the patient, wherein a different or identical number of genes can be selected from the genes listed in each selected table. In certain embodiments, the data set comprises or is derived from gene expression measurement data of the genes listed in each of the one or more Tables selected from Tables 23-1 to 23-28, from the biological sample from the patient. The one or more Tables selected from Tables 23-1 to 23-28 can include at least 1, 2, 3, 4, 5, 6, 7, 8, 9, 10, 11, 12, 13, 14, 15, 16, 17, 18, 19, 20, 21, 22, 23, 24, 25, 26, 27, or 28, or any range there between Tables. In certain embodiments, Tables 23-1 to 23-28 (e.g., 28 Tables) are selected.

In certain embodiments, the data set comprises or is derived from gene expression measurement data of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, or all, or any range or value therebetween, genes selected from the genes listed in each of one or more Tables selected from Tables 25-1 to 25-32, from the biological sample from the patient, wherein the number of genes selected from the genes listed in each selected table may be different or same (e.g., a different or identical number of genes can be selected from the genes listed in each selected table). In certain embodiments, the data set comprises or is derived from gene expression measurement data of an effective number of genes selected from the genes listed in each of the one or more Tables selected from Tables 25-1 to 25-32, from the biological sample from the patient, wherein a different or identical number of genes can be selected from the genes listed in each selected table. In certain embodiments, the data set comprises or is derived from gene expression measurement data of the genes listed in each of the one or more Tables selected from Tables 25-1 to 25-32, from the biological sample from the patient. The one or more Tables selected from Tables 25-1 to 25-32 can include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32, or any range there between Tables. In certain embodiments, Table 25-8 is selected. In certain embodiments, Table 25-31 is selected. In certain embodiments, Tables 25-8 and 25-31 are selected. In certain embodiments, Tables 25-1 to 25-32 (e.g., 32 Tables) are selected.

In certain embodiments, the data set comprises or is derived from gene expression measurement data of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, or all, or any range or value there between, genes selected from the genes listed in each of one or more Tables selected from Tables 28-1 to 28-22, from the biological sample from the patient, wherein the number of genes selected from the genes listed in each selected table may be different or same (e.g., a different or identical number of genes can be selected from the genes listed in each selected table). In certain embodiments, the data set comprises or is derived from gene expression measurement data of an effective number of genes selected from the genes listed in each of the one or more Tables selected from Tables 28-1 to 28-22, from the biological sample from the patient, wherein a different or identical number of genes can be selected from the genes listed in each selected table. In certain embodiments, the data set comprises or is derived from gene expression measurement data of the genes listed in each of the one or more Tables selected from Tables 28-1 to 28-22, from the biological sample from the patient. The one or more Tables selected from Tables 28-1 to 28-22 can include at least 1, 2, 3, 4, 5, 6, 7, 8, 9, 10, 11, 12, 13, 14, 15, 16, 17, 18, 19, 20, 21, or 22, or any range there between Tables. In certain embodiments, Tables 28-1 to 28-22 (e.g., 22 Tables) are selected. In certain embodiments, Tables 28-1, 28-2, 28-3, 28-4, 28-5, 28-6, 28-7, 28-8, 28-12, 28-13, 28-14, 28-15, 28-16, 28-17, 28-18, 28-19, 28-20, 28-21 and 28-22 are selected. In certain embodiments, Tables 28-1, 28-2, 28-3, 28-4, 28-6, 28-7, 28-8, 28-9, 28-10, 28-11, 28-12, 28-13, 28-14, 28-15, 28-16, 28-17, 28-18, 28-20, 28-21 and 28-22 are selected. In certain embodiments, Tables 28-1, 28-2, 28-3, 28-4, 28-5, 28-6, 28-7, 28-8, 28-9, 28-10, 28-11, 28-12, 28-13, 28-14, 28-15, 28-16, 28-17, 28-18, 28-20, 28-21 and 28-22 are selected. In certain embodiments, Tables 28-1 to 28-22 (e.g., 22 Tables) are selected.

Genes selected form each of the selected Tables can be used as effective biomarkers for classifying the LN disease state of the patients.

Selecting effective number of genes from a selected Table can include selecting at least minimum number of genes from the table to obtain desired accuracy, sensitivity, specificity, positive predictive value, and/or negative predictive value in classification of the LN disease state of the patient. Desired accuracy, sensitivity, specificity, positive predictive value, and/or negative predictive value, can be an accuracy, sensitivity, specificity, positive predictive value, and/or negative predictive value described herein. In certain embodiments, the desired accuracy, sensitivity, specificity, positive predictive value, and/or negative predictive value, is at least 80%. In certain embodiments, the desired accuracy, sensitivity, specificity, positive predictive value, and/or negative predictive value, is at least 85%. In certain embodiments, the desired accuracy, sensitivity, specificity, positive predictive value, and/or negative predictive value, is at least 90%. In certain embodiments, the desired accuracy, sensitivity, specificity, positive predictive value, and/or negative predictive value, is at least 95%. In certain embodiments, effective number of genes for a Table can be determined using adjusted rand index (ARI) method. The ARI method can include performing k-Means clustering on randomly selected gene subsets by standard interval based on the total number of genes of a Table. Similarity between two clusters can be measured by adjusted rand index (ARI). As a non-limiting example, the adjusted rand index (ARI) can be calculated between k-Means cluster memberships from the randomly selected gene subsets to the cluster memberships obtained using total number of genes of a Table. The higher the ARI, the similar the cluster memberships and lower the ARI the weaker the cluster memberships, suggesting more genes may be required. The ARI can be calculated to determine the effective number of genes for a Table. In certain embodiments, selecting effective number of genes from a selected Table can include selecting at least 60%, 70%, 80%, 90%, or all genes listed in the selected Table. In certain embodiments, selecting effective number of genes from a selected Table can include selecting at least 60% of the genes listed in the selected Table. In certain embodiments, selecting effective number of genes from a selected Table can include selecting at least 70% of the genes listed in the selected Table. In certain embodiments, selecting effective number of genes from a selected Table can include selecting at least 80% of the genes listed in the selected Table. In certain embodiments, selecting effective number of genes from a selected Table can include selecting at least 90% of the genes listed in the selected Table. In certain embodiments, selecting effective number of genes from a selected Table can include selecting all the genes listed in the selected Table. In certain embodiments, selecting effective number of genes from a selected Table can include selecting at least about 60%, 65%, 70%, 75%, 80%, 85%, 90%, 95%, 96%, 97%, 98%, 99% or 100% of the genes in the Table. In certain embodiments, selecting an effective number of genes from a selected Table can include selecting at least about 60%, 65%, 70%, 75%, 80%, 85%, 90%, 95%, 96%, 97%, 98%, 99% or 100% of the genes in the Table, where the Table contains 100 or more genes. In certain embodiments, selecting effective number of genes from a selected Table can include selecting at least 70%, genes from the Table, where the Table contains 100 or more genes. In certain embodiments, selecting effective number of genes from a selected Table can include selecting at least about 80%, 90%, 95%, 96%, 97%, 98%, 99%, or 100% of the genes in the Table, where the Table contains less than 100 genes. In certain embodiments, selecting effective number of genes from a selected Table can include selecting all genes from the Table, where the Table contains less than 100 genes. In certain embodiments, collinear genes (such as with r>0.9, >0.8, >0.7, or >0.6) are be removed from the gene set forming the effective number of genes. In some embodiments, an effective number of genes in a Table disclosed herein comprises about 60 percent to about 100 percent of the genes in the Table. In some embodiments, an effective number of genes in a Table disclosed herein comprises about 60 percent to about 65 percent, about 60 percent to about 70 percent, about 60 percent to about 75 percent, about 60 percent to about 80 percent, about 60 percent to about 85 percent, about 60 percent to about 90 percent, about 60 percent to about 95 percent, about 60 percent to about 97 percent, about 60 percent to about 98 percent, about 60 percent to about 99 percent, about 60 percent to about 100 percent, about 65 percent to about 70 percent, about 65 percent to about 75 percent, about 65 percent to about 80 percent, about 65 percent to about 85 percent, about 65 percent to about 90 percent, about 65 percent to about 95 percent, about 65 percent to about 97 percent, about 65 percent to about 98 percent, about 65 percent to about 99 percent, about 65 percent to about 100 percent, about 70 percent to about 75 percent, about 70 percent to about 80 percent, about 70 percent to about 85 percent, about 70 percent to about 90 percent, about 70 percent to about 95 percent, about 70 percent to about 97 percent, about 70 percent to about 98 percent, about 70 percent to about 99 percent, about 70 percent to about 100 percent, about 75 percent to about 80 percent, about 75 percent to about 85 percent, about 75 percent to about 90 percent, about 75 percent to about 95 percent, about 75 percent to about 97 percent, about 75 percent to about 98 percent, about 75 percent to about 99 percent, about 75 percent to about 100 percent, about 80 percent to about 85 percent, about 80 percent to about 90 percent, about 80 percent to about 95 percent, about 80 percent to about 97 percent, about 80 percent to about 98 percent, about 80 percent to about 99 percent, about 80 percent to about 100 percent, about 85 percent to about 90 percent, about 85 percent to about 95 percent, about 85 percent to about 97 percent, about 85 percent to about 98 percent, about 85 percent to about 99 percent, about 85 percent to about 100 percent, about 90 percent to about 95 percent, about 90 percent to about 97 percent, about 90 percent to about 98 percent, about 90 percent to about 99 percent, about 90 percent to about 100 percent, about 95 percent to about 97 percent, about 95 percent to about 98 percent, about 95 percent to about 99 percent, about 95 percent to about 100 percent, about 97 percent to about 98 percent, about 97 percent to about 99 percent, about 97 percent to about 100 percent, about 98 percent to about 99 percent, about 98 percent to about 100 percent, or about 99 percent to about 100 percent of the genes in the Table. In some embodiments, an effective number of genes in a Table disclosed herein comprises about 60 percent, about 65 percent, about 70 percent, about 75 percent, about 80 percent, about 85 percent, about 90 percent, about 95 percent, about 97 percent, about 98 percent, about 99 percent, or about 100 percent of the genes in the Table. In some embodiments, an effective number of genes in a Table disclosed herein comprises at least about 60 percent, about 65 percent, about 70 percent, about 75 percent, about 80 percent, about 85 percent, about 90 percent, about 95 percent, about 97 percent, about 98 percent, or about 99 percent of the genes in the Table.

In certain embodiments, a minimum number of Tables are selected (e.g., from Tables 23-1 to 23-28, or from Tables 25-1 to 25-32, or from Tables 26-1 to 26-60, or from Tables 27-1 to 27-48, or from Tables 28-1 to 28-22) such that the method can classify/identify all four endotypes (acute LN, transitional LN, chronic group I LN and chronic group II LN) of LN disease state. In certain embodiments, a minimum number of Tables are selected (e.g., from Tables 23-1 to 23-28, or from Tables 25-1 to 25-32, or from Tables 26-1 to 26-60, or from Tables 27-1 to 27-48, or from Tables 28-1 to 28-22) such that the method can classify the LN disease state of the patient with a desired accuracy, sensitivity, specificity, positive predictive value, and/or negative predictive value. The desired accuracy, sensitivity, specificity, positive predictive value, and/or negative predictive value, can be an accuracy, sensitivity, specificity, positive predictive value, and/or negative predictive value described herein. In certain embodiments, the desired accuracy, sensitivity, specificity, positive predictive value, and/or negative predictive value, is at least 80%. In certain embodiments, the desired accuracy, sensitivity, specificity, positive predictive value, and/or negative predictive value, is at least 85%. In certain embodiments, the desired accuracy, sensitivity, specificity, positive predictive value, and/or negative predictive value, is at least 90%. In certain embodiments, the desired accuracy, sensitivity, specificity, positive predictive value, and/or negative predictive value, is at least 95%.

The data set can be generated from the biological sample from the patient. For example, nucleic acid molecules of the patient in the biological sample can be assessed to obtain the data set. In certain embodiments, the gene expression measurements of the at least 2 genes from the biological sample can be performed using any suitable method known to those of skill in the art including but not limited to DNA sequencing, RNA sequencing, microarray, RNA-Seq, qPCR, northern blotting, fluorescence in situ hybridization, serial analysis of gene expression, tiling arrays or any combination thereof, to obtain the data set. In certain embodiments, the gene expression measurements of the at least 2 genes in the biological sample can be performed using RNA-Seq. RNA-Seq can include single cell RNA-Seq, and/or bulk RNA-Seq. In certain embodiments, the gene expression measurements of the at least 2 genes in the biological sample can be performed using microarray analysis. In certain embodiments, the data set is derived from the gene expression measurement data from the biological sample, wherein the gene expression measurement data is analyzed using a suitable data analysis tool including but not limited to BIG-C™ big data analysis tool, an I-Scope™ big data analysis tool, a T-Scope™ big data analysis tool, a CellScan big data analysis tool, an MS (Molecular Signature) Scoring™ analysis tool, gene set variation analysis (GSVA), gene set enrichment analysis (GSEA), enrichment algorithm, Z score, multiscale embedded gene co-expression network analysis (MEGENA), weighted gene co-expression network analysis (WGCNA), differential expression analysis, log 2 expression analysis, or any combination thereof, to obtain the dataset. In certain embodiments, the data set is derived from the gene expression measurement data using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, Z-score, log 2 expression analysis, or any combination thereof. In certain embodiments, the data set is derived from the gene expression measurement data using gene set variation analysis (GSVA). In certain embodiments, the method comprises obtaining and/or deriving the biological sample from the patient. In certain embodiments, the method comprises analyzing the biological sample to obtain the gene expression measurement data from the biological sample. In certain embodiments, the method comprises analyzing the gene expression measurement data to obtain the dataset. In certain embodiments, the method comprises obtaining and/or deriving the biological sample from the patient, and/or analyzing the biological sample to obtain the gene expression measurement data from the biological sample. In certain embodiments, the method comprises obtaining and/or deriving the biological sample from the patient, analyzing the biological sample to obtain the gene expression measurement data from the biological sample, and/or analyzing the gene expression measurement data to obtain the dataset.

In certain embodiments, the data set is derived from the gene expression measurement data, and the data set comprises one or more enrichment scores of the patient. The one or more enrichment scores of the patient can be generated based on the one or more Tables selected from Tables 19-1 to 19-36, Tables 19A-1 to 19A-36, Tables 23-1 to 23-28, Tables 25-1 to 25-32, Tables 26-1 to 26-60, Tables 27-1 to 27-48, and Tables 28-1 to 28-22 wherein for each selected Table, at least one enrichment score of the patient is generated based on enrichment of expression of the at least 2 genes (or one or more human orthologs thereof) selected from the genes listed in the selected Table, in the biological sample. The one or more enrichment scores can contain the at least one enrichment score generated from each of the selected Table. The at least 2 genes selected from a respective selected Table, can form the input gene set for generating the at least one enrichment score from the respective selected Table. The at least 2 genes of the data set can comprise the at least 2 genes selected from each of the selected table. In certain embodiments, the data set can be derived from the gene expression measurements of the genes selected from the selected Tables using GSVA, and the data set comprises one or more enrichment scores of the patient. In certain embodiments, for each selected Table, the at least one enrichment score of the patient is generated based on enrichment of expression of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 203, or all, any range or value there between genes (or one or more human orthologs thereof) selected from the genes listed in the respective Table, in the biological sample, wherein number of genes selected from different selected Tables can be the same or different. In certain embodiments, for each selected Table, the at least one enrichment score of the patient is generated based on enrichment of expression of an effective number of genes (or one or more human orthologs thereof) selected from the genes listed in the selected Table, in the biological sample, wherein a different or identical number of genes can be selected from the genes listed in each selected table. In certain embodiments, for each selected Table, the at least one enrichment score of the patient is generated based on enrichment of expression of all the genes (or one or more human orthologs thereof) listed in the selected Table, in the biological sample. The genes selected from a respective selected Table (or one or more human orthologs thereof), can form the input gene set for generating the at least one enrichment score of the patient based on the respective selected Table. The at least one enrichment score based on a selected Table can be generated based on enrichment of the input gene set (e.g., containing genes selected from the selected Table, e.g., at least 2 genes, effective number of genes, or all the genes selected from the selected Table) based on the selected Table, in the biological sample. Enrichment can be determined with respect to a reference data set, as described herein. In a non-limiting example, the one or more Tables selected comprise Tables: 23-1 and 23-2, and effective number of genes are selected from the genes listed in each of the Tables selected, and the dataset comprises the one or more enrichment scores of the patient, thereby the one or more enrichment scores of the patient comprise at least one enrichment score generated based on Table 23-1, and at least one enrichment score generated based on Table 23-2, wherein the at least one enrichment score generated based on Table 23-1 is generated based on enrichment of the input gene set (e.g., containing the effective number of genes selected from the genes listed in Table 23-1) based on Table 23-1 in the biological sample, and the at least one enrichment score generated based on Table 23-2 is generated based on enrichment of the input gene set (e.g., containing the effective number of genes selected from the genes listed in Table 23-2) based on Table 23-2 in the biological sample. In certain embodiments, one enrichment score is generated from each of the selected Tables. In certain embodiments, the dataset comprises the one or more enrichment scores of the patients, and analyzing the data set comprises analyzing the one or more enrichment scores of the patient to classify the LN disease state of the patient. In certain embodiments, the one or more enrichment scores of the patients are analyzed, to classify the LN disease state of the patient. The enrichment score can be generated using any suitable method, including but not limited to GSEA and GSVA. In certain embodiments, the enrichment scores are generated based on GSVA, and the enrichment scores are GSVA scores.

In certain embodiments, the one or more enrichment scores of the patient are generated based on one or more Tables selected from Tables 19-1 to 19-36. In certain embodiments, the one or more enrichment scores of the patient are generated based on one or more Tables selected from Tables 19-1 to 19-36, and the one or more Tables comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36, or any range therebetween Tables. In certain embodiments, Tables 19-1 to 19-36 (e.g., 36 Tables) are selected.

In certain embodiments, the one or more enrichment scores of the patient are generated based on one or more Tables selected from Tables 19A-1 to 19A-36. In certain embodiments, the one or more enrichment scores of the patient are generated based on one or more Tables selected from Tables 19A-1 to 19A-36, and the one or more Tables comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36, or any range therebetween Tables. In certain embodiments, Tables 19A-1 to 19A-36 (e.g., 36 Tables) are selected.

In certain embodiments, the one or more enrichment scores of the patient are generated based on one or more Tables selected from Tables 26-1 to 26-60. In certain embodiments, the one or more enrichment scores of the patient are generated based on one or more Tables selected from Tables 26-1 to 26-60, and the one or more Tables comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60, or any range therebetween Tables. In certain embodiments, Tables 26-1 to 26-60 (e.g., 60 Tables) are selected.

In certain embodiments, the one or more enrichment scores of the patient are generated based on one or more Tables selected from Tables 27-1 to 27-48. In certain embodiments, the one or more enrichment scores of the patient are generated based on one or more Tables selected from Tables 27-1 to 27-48, and the one or more Table comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48, or any range therebetween Tables. In certain embodiments, Tables 27-1 to 27-48 (e.g., 48 Tables) are selected.

In certain embodiments, the one or more enrichment scores of the patient are generated based on one or more Tables selected from Tables 23-1 to 23-28. In certain embodiments, the one or more enrichment scores of the patient are generated based on one or more Tables selected from Tables 23-1 to 23-28, and the one or more Tables comprises at least 1, 2, 3, 4, 5, 6, 7, 8, 9, 10, 11, 12, 13, 14, 15, 16, 17, 18, 19, 20, 21, 22, 23, 24, 25, 26, 27, or 28, or any range therebetween Tables. In certain embodiments, Tables 23-1 to 23-28 (e.g., 28 Tables) are selected.

In certain embodiments, the one or more enrichment scores of the patient are generated based on one or more Tables selected from Tables 25-1 to 25-32. In certain embodiments, the one or more enrichment scores of the patient are generated based on one or more Tables selected from Tables 25-1 to 25-32, and the one or more Tables comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or 32, or any range therebetween Tables. In certain embodiments, Tables 25-1 to 25-32 (e.g., 32 Tables) are selected.

In certain embodiments, the one or more enrichment scores of the patient are generated based on one or more Tables selected from Tables 28-1 to 28-22. In certain embodiments, the one or more enrichment scores of the patient are generated based on one or more Tables selected from Tables 28-1 to 28-22, and the one or more Tables comprise at least 1, 2, 3, 4, 5, 6, 7, 8, 9, 10, 11, 12, 13, 14, 15, 16, 17, 18, 19, 20, 21, or 22, or any range therebetween Tables. In certain embodiments, Tables 28-1, 28-2, 28-3, 28-4, 28-5, 28-6, 28-7, 28-8, 28-12, 28-13, 28-14, 28-15, 28-16, 28-17, 28-18, 28-19, 28-20, 28-21 and 28-22 are selected. In certain embodiments, Tables 28-1, 28-2, 28-3, 28-4, 28-6, 28-7, 28-8, 28-9, 28-10, 28-11, 28-12, 28-13, 28-14, 28-15, 28-16, 28-17, 28-18, 28-20, 28-21 and 28-22 are selected. In certain embodiments, Tables 28-1, 28-2, 28-3, 28-4, 28-5, 28-6, 28-7, 28-8, 28-9, 28-10, 28-11, 28-12, 28-13, 28-14, 28-15, 28-16, 28-17, 28-18, 28-20, 28-21 and 28-22 are selected. In certain embodiments, Tables 28-1 to 28-22 (e.g., 22 Tables) are selected.

In certain embodiments, the data set is derived from the gene expression measurement data using GSVA. In certain embodiments, the data set is derived from the gene expression measurement data using GSVA, and the data set comprises one or more GSVA scores of the patient. The one or more GSVA scores of the patient can be generated based on the one or more Tables selected from Tables 19-1 to 19-36, Tables 19A-1 to 19A-36, Tables 23-1 to 23-28, Tables 25-1 to 25-32, Tables 26-1 to 26-60, Tables 27-1 to 27-48, and Tables 28-1 to 28-22, wherein for each selected Table, at least one GSVA score of the patient is generated based on enrichment of expression of the at least 2 genes (or one or more human orthologs thereof) selected from the genes listed in the selected Table, in the biological sample. The one or more GSVA scores can contain the at least one GSVA score generated from each of the selected Table. The at least 2 genes (or one or more human orthologs thereof) selected from a respective selected Table, can form the input gene set for generating the at least one GSVA score from the respective selected Table, using GSVA. The at least 2 genes of the data set can comprise the at least 2 genes selected from each of the selected table. In certain embodiments, for each selected Table, the at least one GSVA score of the patient is generated based on enrichment of expression of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 203, or all, any range or value there between genes (or one or more human orthologs thereof) selected from the genes listed in the respective Table, in the biological sample, wherein number of genes selected from different selected Tables can be the same or different. In certain embodiments, for each selected Table, the at least one GSVA score of the patient is generated based on enrichment of expression of an effective number of genes (or one or more human orthologs thereof) selected from the genes listed in the selected Table, in the biological sample, wherein a different or identical number of genes can be selected from the genes listed in each selected table. In certain embodiments, for each selected Table, the at least one GSVA score of the patient is generated based on enrichment of expression of all the genes (or one or more human orthologs thereof) listed in the selected Table, in the biological sample. The genes selected from a respective selected Table (or one or more human orthologs thereof), can form the input gene set for generating the at least one GSVA score of the patient based on the respective selected Table, using GSVA. The at least one GSVA score based on a selected Table can be generated based on enrichment of the input gene set (e.g., containing the genes selected from the selected Table, e.g., at least 2 genes, effective number of genes, or all the genes selected from the selected Table) based on the selected Table, in the biological sample. Enrichment can be determined with respect to a reference data set, as described herein. In a non-limiting example, the one or more Tables selected comprise Tables: 23-1 and 23-2, and effective number of genes are selected from the genes listed in each of the Table selected, and the dataset comprises the one or more GSVA scores of the patient, thereby the one or more GSVA scores of the patient comprise at least one GSVA score generated based on Table 23-1, and at least one GSVA score generated based on Table 23-2, wherein the at least one GSVA score generated based on Table 23-1 is generated based on enrichment of the input gene set (e.g., containing the effective number of genes selected from the genes listed in Table 23-1) based on Table 23-1 in the biological sample, and the at least one GSVA score generated based on Table 23-2 is generated based on enrichment of the input gene set (e.g., containing the effective number of genes selected from the genes listed in Table 23-2) based on Table 23-2 in the biological sample. In certain embodiments, one GSVA score is generated from each of the selected Tables. In certain embodiments, the dataset comprises the one or more GSVA scores of the patients, and analyzing the data set comprises analyzing the one or more GSVA scores of the patient to classify the LN disease state of the patient. In certain embodiments, the one or more GSVA scores of the patients are analyzed, to classify the LN disease state of the patient

In certain embodiments, the one or more GSVA scores of the patient are generated based on one or more Tables selected from Tables 19-1 to 19-36. In certain embodiments, the one or more GSVA scores of the patient are generated based on one or more Tables selected from Tables 19-1 to 19-36, and the one or more Tables comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36, or any range therebetween Tables. In certain embodiments, Tables 19-1 to 19-36 (e.g., 36 Tables) are selected.

In certain embodiments, the one or more GSVA scores of the patient are generated based on one or more Tables selected from Tables 19A-1 to 19A-36. In certain embodiments, the one or more GSVA scores of the patient are generated based on one or more Tables selected from Tables 19A-1 to 19A-36, and the one or more Tables comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36, or any range therebetween Tables. In certain embodiments, Tables 19A-1 to 19A-36 (e.g., 36 Tables) are selected.

In certain embodiments, the one or more GSVA scores of the patient are generated based on one or more Tables selected from Tables 26-1 to 26-60. In certain embodiments, the one or more GSVA scores of the patient are generated based on one or more Tables selected from Tables 26-1 to 26-60, and the one or more Tables comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60, or any range therebetween Tables. In certain embodiments, Tables 26-1 to 26-60 (e.g., 60 Tables) are selected.

In certain embodiments, the one or more GSVA scores of the patient are generated based on one or more Tables selected from Tables 27-1 to 27-48. In certain embodiments, the one or more GSVA scores of the patient are generated based on one or more Tables selected from Tables 27-1 to 27-48, and the one or more Tables comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48, or any range therebetween Tables. In certain embodiments, Tables 27-1 to 27-48 (e.g., 48 Tables) are selected.

In certain embodiments, the one or more GSVA scores of the patient are generated based on one or more Tables selected from Tables 23-1 to 23-28. In certain embodiments, the one or more GSVA scores of the patient are generated based on one or more Tables selected from Tables 23-1 to 23-28, and the one or more Tables comprise at least 1, 2, 3, 4, 5, 6, 7, 8, 9, 10, 11, 12, 13, 14, 15, 16, 17, 18, 19, 20, 21, 22, 23, 24, 25, 26, 27, or 28, or any range therebetween Tables. In certain embodiments, Tables 23-1 to 23-28 (e.g., 28 Tables) are selected.

In certain embodiments, the one or more GSVA scores of the patient are generated based on one or more Tables selected from Tables 25-1 to 25-32. In certain embodiments, the one or more GSVA scores of the patient are generated based on one or more Tables selected from Tables 25-1 to 25-32, and the one or more Tables comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or 32, or any range therebetween Tables. In certain embodiments, Tables 25-1 to 25-32 (e.g., 32 Tables) are selected.

In certain embodiments, the one or more GSVA scores of the patient are generated based on one or more Tables selected from Tables 28-1 to 28-22. In certain embodiments, the one or more GSVA scores are generated based on one or more Tables selected from Tables 28-1 to 28-22, and the one or more Tables comprise at least 1, 2, 3, 4, 5, 6, 7, 8, 9, 10, 11, 12, 13, 14, 15, 16, 17, 18, 19, 20, 21, or 22, or any range there between Tables. In certain embodiments, Tables 28-1, 28-2, 28-3, 28-4, 28-5, 28-6, 28-7, 28-8, 28-12, 28-13, 28-14, 28-15, 28-16, 28-17, 28-18, 28-19, 28-20, 28-21 and 28-22 are selected. In certain embodiments, Tables 28-1, 28-2, 28-3, 28-4, 28-6, 28-7, 28-8, 28-9, 28-10, 28-11, 28-12, 28-13, 28-14, 28-15, 28-16, 28-17, 28-18, 28-20, 28-21 and 28-22 are selected. In certain embodiments, Tables 28-1, 28-2, 28-3, 28-4, 28-5, 28-6, 28-7, 28-8, 28-9, 28-10, 28-11, 28-12, 28-13, 28-14, 28-15, 28-16, 28-17, 28-18, 28-20, 28-21 and 28-22 are selected. In certain embodiments, Tables 28-1 to 28-22 (e.g., 22 Tables) are selected.

In certain embodiments, analyzing the dataset comprises analyzing gene expression of one or more gene sets formed based on the one or more Tables selected from Tables 19-1 to 19-36, Tables 19A-1 to 19A-36, Tables 23-1 to 23-28, Tables 25-1 to 25-32, Tables 26-1 to 26-60, Tables 27-1 to 27-48, and Tables 28-1 to 28-22, wherein genes (or one or more human orthologs thereof) selected from each of the selected Table can form a gene set of the one or more gene sets. Genes (or one or more human orthologs thereof) selected from different selected Tables can form different gene sets of the one or more gene sets. The dataset can comprise the gene expression measurement data of the one or more gene sets. The at least 2 genes (or one or more human orthologs thereof) of the dataset can comprise the genes within the one or more gene sets. The one or more Tables selected (e.g., based on which the one or more gene sets are formed) can comprise the selected Tables as described above or elsewhere herein. For a selected Table the genes selected from the selected Table can comprise the selected genes as described above or elsewhere herein, such as at least 2 genes, effective number of genes, and/or all genes from the selected Table. In certain embodiments, for each selected Table the genes selected (e.g., that forms the gene set based on the selected Table) comprise at least 2 genes (or one or more human orthologs thereof) selected from the genes listed in the selected Table, wherein the number of genes selected from different selected Tables can be the same or different. In certain embodiments, for each selected Table the genes selected (e.g., that forms the gene set based on the selected Table) comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or all genes (or one or more human orthologs thereof) selected from the genes listed in the selected Table, wherein the number of genes selected from different selected Tables can be the same or different. In certain embodiments, for each selected Table the genes selected (e.g., that forms the gene set based on the selected Table) comprise effective number of genes (or one or more human orthologs thereof) selected from the genes listed in the selected Table, wherein the number of genes selected from different selected Tables can be the same or different. In certain embodiments, for each selected Table the genes selected (e.g., that forms the gene set based on the selected Table) comprise all the genes (or one or more human orthologs thereof) listed in the selected Table. Each of the one or more gene sets can be generated based on one of the one or more selected Tables, wherein for each selected Table the genes selected (e.g., at least 2 genes, effective number of genes, and/or all genes) from the selected Table (or one or more human orthologs thereof) forms a gene set of the one or more gene set. In a non-limiting example, the one or more Tables selected comprise Tables: 23-1, 23-2 and 23-3, and effective number of genes are selected from each of the Table selected, and the data set comprises gene expression measurement data of one or more gene sets formed based on the one or more Tables selected, thereby the one or more gene sets comprise a gene set formed based on Table 23-1, a gene set formed based on Table 23-2, and a gene set formed based on Table 23-3, wherein the gene set formed based on Table 23-1 comprises effective number of genes selected from the genes listed in Table 23-1, the gene set formed based on Table 23-2 comprises effective number of genes selected from the genes listed in Table 23-2, and the gene set formed based on Table 23-3 comprises effective number of genes selected from the genes listed in Table 23-3. In certain embodiments, analyzing gene expression (e.g., in the biological sample) of a gene set (e.g., of the one or more gene sets) can include analyzing module eigengenes (MEs) of the gene set (e.g., forming a module). In certain embodiments, the dataset comprises the gene expression measurement data of the one or more gene sets, and analyzing the dataset comprises analyzing gene expression of one or more gene sets to classify the LN disease of the patient. In certain embodiments, the gene expression (e.g., in the biological sample) of the one or more gene sets can be analyzed to classify the LN disease of the patient. In certain embodiments, the one or more gene sets are generated based on one or more Tables selected from Tables 19-1 to 19-36. In certain embodiments, the one or more gene sets are generated based on one or more Tables selected from Tables 19-1 to 19-36, and the one or more Tables comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36, or any range therebetween Tables. In certain embodiments, Tables 19-1 to 19-36 (e.g., 36 Tables) are selected. In certain embodiments, the one or more gene sets are generated based on one or more Tables selected from Tables 19A-1 to 19A-36. In certain embodiments, the one or more gene sets are generated based on one or more Tables selected from Tables 19A-1 to 19A-36, and the one or more Tables comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36, or any range therebetween Tables. In certain embodiments, Tables 19A-1 to 19A-36 (e.g., 36 Tables) are selected. In certain embodiments, the one or more gene sets are generated based on one or more Tables selected from Tables 26-1 to 26-60. In certain embodiments, the one or more gene sets are generated based on one or more Tables selected from Tables 26-1 to 26-60, and the one or more Tables comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60, or any range therebetween Tables. In certain embodiments, Tables 26-1 to 26-60 (e.g., 60 Tables) are selected. In certain embodiments, the one or more gene sets are generated based on one or more Tables selected from Tables 27-1 to 27-48. In certain embodiments, the one or more gene sets are generated based on one or more Tables selected from Tables 27-1 to 27-48, and the one or more Tables comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48, or any range therebetween Tables. In certain embodiments, Tables 27-1 to 27-48 (e.g., 48 Tables) are selected. In certain embodiments, the one or more gene sets are generated based on one or more Tables selected from Tables 23-1 to 23-28. In certain embodiments, the one or more gene sets are generated based on one or more Tables selected from Tables 23-1 to 23-28, and the one or more Tables comprise at least 1, 2, 3, 4, 5, 6, 7, 8, 9, 10, 11, 12, 13, 14, 15, 16, 17, 18, 19, 20, 21, 22, 23, 24, 25, 26, 27, or 28, or any range there between Tables. In certain embodiments, Tables 23-1 to 23-28 (e.g., 28 Tables) are selected. In certain embodiments, the one or more gene sets are generated based on one or more Tables selected from Tables 25-1 to 25-32. In certain embodiments, the one or more gene sets are generated based on one or more Tables selected from Tables 25-1 to 25-32, and the one or more Tables comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or 32, or any range therebetween Tables. In certain embodiments, Tables 25-1 to 25-32 (e.g., 32 Tables) are selected. In certain embodiments, the one or more gene sets are generated based on one or more Tables selected from Tables 28-1 to 28-22. In certain embodiments, the one or more gene sets generated based on one or more Tables selected from Tables 28-1 to 28-22, and the one or more Tables comprise at least 1, 2, 3, 4, 5, 6, 7, 8, 9, 10, 11, 12, 13, 14, 15, 16, 17, 18, 19, 20, 21, or 22, or any range there between Tables. In certain embodiments, Tables 28-1, 28-2, 28-3, 28-4, 28-5, 28-6, 28-7, 28-8, 28-12, 28-13, 28-14, 28-15, 28-16, 28-17, 28-18, 28-19, 28-20, 28-21 and 28-22 are selected. In certain embodiments, Tables 28-1, 28-2, 28-3, 28-4, 28-6, 28-7, 28-8, 28-9, 28-10, 28-11, 28-12, 28-13, 28-14, 28-15, 28-16, 28-17, 28-18, 28-20, 28-21 and 28-22 are selected. In certain embodiments, Tables 28-1, 28-2, 28-3, 28-4, 28-5, 28-6, 28-7, 28-8, 28-9, 28-10, 28-11, 28-12, 28-13, 28-14, 28-15, 28-16, 28-17, 28-18, 28-20, 28-21 and 28-22 are selected. In certain embodiments, Tables 28-1 to 28-22 (e.g., 22 Tables) are selected.

In certain embodiments, analyzing the data set comprises providing the data set as an input to a machine-learning model to classify the LN disease state of the patient. The machine-learning model can generate an inference indicative of the LN disease state of the patient, based at least on the data set. The method can classify the LN disease state of the patient based on the inference. In certain embodiments, the data set comprises the one or more enrichment scores of the patient, and the machine-learning model generates the inference based at least on the one or more enrichment scores. In certain embodiments, the data set comprises the one or more GSVA scores of the patient, and the machine-learning model generates the inference based at least on the one or more GSVA scores. In certain embodiments, the data set comprises gene expression measurement data (such as MEs) of the one or more gene sets, and the machine-learning model generates the inference based at least on the gene expression (such as MEs) of the one or more gene sets. In certain embodiments, the method further comprises receiving, as an output of the machine-learning model, the inference; and/or electronically outputting a report indicating of the LN disease state of the patient based on the inference. The machine learning model can be a trained machine learning model.

The trained machine learning model can generates the inference based at least on comparing the data set to a reference data set. The reference data set can comprise and/or be derived from gene expression measurements from reference biological samples of at least 2 genes (or human orthologs thereof) selected from the genes listed in Tables 19-1 to 19-36, Tables 19A-1 to 19A-36, Table 20, Table 21, Table 22, Tables 23-1 to 23-28, Tables 25-1 to 25-32, Tables 26-1 to 26-60, Tables 27-1 to 27-48 and Tables 28-1 to 28-22. In certain embodiments, the at least 2 genes expression measurements of which, the reference data set is comprised of and/or derived from are selected from the genes listed in Tables 23-1 to 23-28. In certain embodiments, the at least 2 genes expression measurements of which, the reference data set is comprised of and/or derived from are selected from the genes listed in Tables 25-1 to 25-32. In certain embodiments, the at least 2 genes expression measurements of one or more human orthologs of which, the reference data set is comprised of and/or derived from are selected from the genes listed in Tables 19-1 to 19-36. In certain embodiments, the at least 2 genes expression measurements of which, the reference data set is comprised of and/or derived from are selected from the genes listed in Tables 19A-1 to 19A-36. In certain embodiments, the at least 2 genes, expression measurements of which, the reference data set is comprised of and/or derived from are selected from the genes listed in Tables 26-1 to 26-60. In certain embodiments, the at least 2 genes, expression measurements of which, the reference data set is comprised of and/or derived from are selected from the genes listed in Tables 27-1 to 27-48. In certain embodiments, the at least 2 genes, expression measurements of which, the reference data set is comprised of and/or derived from are selected from the genes listed in Tables 28-1 to 28-22. The at least 2 genes gene expression measurements of which, the reference data set is comprised of and/or derived from, and the at least 2 genes gene expression measurements of which, the data set is comprised of and/or derived from can at least partially overlap (e.g., one or more genes can be the same). In certain embodiments, the selected genes, the gene expression measurements of which (or one or more human orthologs thereof) are comprised by the data set, and the selected genes the gene expression measurements of which are comprised by the reference data set are same. In certain embodiments, selected genes of the dataset, and selected genes of the reference dataset are same. In certain embodiments, selected genes of the dataset, and selected genes of the reference dataset are same, and can be any selected set of genes e.g., of the data set, as described above or elsewhere herein. The Tables selected, and genes selected from a selected Table for the data set and the reference data set can be the same, and can be as described (e.g., for the data set) herein. In certain embodiments, the reference data set contains gene expression (such as MEs) from the reference biological samples of the one or more gene sets formed based on the selected Tables, wherein the one or more gene sets of the reference dataset can be the same (e.g., formed based on the same selected Tables and contains same genes selected from the selected Tables) as the one or more gene sets of the dataset, as described above. In certain embodiments, the machine learning model is trained based on gene expression (such as MEs) from the reference biological samples, of the one or more gene sets, and analyzing the data set include providing the gene expression (such as MEs) from the biological sample, of the one or more gene sets, to the trained machine learning model. The reference biological samples can be obtained or derived from a plurality of reference subjects. In certain embodiments, the reference biological samples comprise a first plurality of reference biological samples obtained or derived from reference subjects having LN, and a second plurality of reference biological samples obtained or derived from reference subjects not having LN. In certain embodiments, the reference biological samples comprise a first plurality of reference biological samples obtained or derived from reference subjects having acute LN, a second plurality of reference biological samples obtained or derived from reference subjects having transitional LN, a third plurality of reference biological samples obtained or derived from reference subjects having chronic LN, and/or a fourth plurality of reference biological samples obtained or derived from reference subjects not having LN. In certain embodiments, the reference biological samples comprise a first plurality of reference biological samples obtained or derived from reference subjects having acute LN, a second plurality of reference biological samples obtained or derived from reference subjects having transitional LN, a third plurality of reference biological samples obtained or derived from reference subjects having chronic LN, and a fourth plurality of reference biological samples obtained or derived from reference subjects not having LN. In certain embodiments, the reference biological samples comprise a first plurality of reference biological samples obtained or derived from reference subjects having acute LN, a second plurality of reference biological samples obtained or derived from reference subjects having transitional LN, and a third plurality of reference biological samples obtained or derived from reference subjects having chronic LN. In certain embodiments, the reference biological samples comprise a first plurality of reference biological samples obtained or derived from reference subjects having acute LN, a second plurality of reference biological samples obtained or derived from reference subjects having transitional LN, a third plurality of reference biological samples obtained or derived from reference subjects having chronic group I LN, a fourth plurality of reference biological samples obtained or derived from reference subjects having chronic group II LN, and/or a fifth plurality of reference biological samples obtained or derived from reference subjects not having LN. In certain embodiments, the reference biological samples comprise a first plurality of reference biological samples obtained or derived from reference subjects having acute LN, a second plurality of reference biological samples obtained or derived from reference subjects having transitional LN, a third plurality of reference biological samples obtained or derived from reference subjects having chronic group I LN, and a fourth plurality of reference biological samples obtained or derived from reference subjects having chronic group II LN. In certain embodiments, the reference biological samples comprise a first plurality of reference biological samples obtained or derived from reference subjects having acute LN, a second plurality of reference biological samples obtained or derived from reference subjects having transitional LN, a third plurality of reference biological samples obtained or derived from reference subjects having chronic group I LN, a fourth plurality of reference biological samples obtained or derived from reference subjects having chronic group II LN, and a fifth plurality of reference biological samples obtained or derived from reference subjects not having LN. The trained machine learning model can be trained (e.g., obtained by training) using the reference data set. A first portion of the reference data set can be used as training data set, and a second portion of the reference data set can be used as validation data set. One-vs.-one and one-vs.-rest multi-class classifications with leave-one-out cross-validation can employed to infer reference a subject's LN disease state to one of the five groups, e.g., acute, transitional, chronic group I chronic group II, LN disease state and not having LN. In certain embodiments, 0 to 25 fold, such as 0, 1, 2, 3, 4, 5, 6, 7, 8, 9, 10, 11, 12, 13, 14, 15, 16, 17, 18, 19, 20, 21, 22, 23, 24 or 25 fold cross-validation is used. In certain embodiments, 6 fold cross-validation is used. In certain embodiments, 10 fold cross-validation is used. In certain embodiments, oversampling or undersampling correction is made during training of the machine learning model. Synthetic Minority Oversampling Technique (SMOTE) can be applied on the training data to handle class imbalances. In certain embodiments low intensity genes (e.g., with IQR<0) in the reference dataset, were filtered out during training the machine learning model using the reference data set, and from the dataset during analysis of the dataset using the trained machine learning model. The trained machine learning model can be trained to generate an inference indicative of the LN disease state of a reference subject, based at least on an individual data set comprising and/or derived from gene expression measurement data of the at least 2 genes (e.g., of the reference data set) from a reference biological sample from the reference subject. In certain embodiments, the machine learning model can be trained using a method and/or reference dataset as described in the Examples. In certain embodiments, the reference data set can be derived from the gene expression measurement data of the reference biological samples, wherein the gene expression measurement data is analyzed using a suitable data analysis tool including but not limited to a BIG-C™ big data analysis tool, an I-Scope™ big data analysis tool, a T-Scope™ big data analysis tool, a CellScan big data analysis tool, an MS (Molecular Signature) Scoring™ analysis tool, gene set variation analysis (GSVA), gene set enrichment analysis (GSEA), enrichment algorithm, Z score, multiscale embedded gene co-expression network analysis (MEGENA), weighted gene co-expression network analysis (WGCNA), differential expression analysis, log 2 expression analysis, or any combination thereof, to obtain the reference data set. In certain embodiments, the gene expression measurement data of the reference biological samples can be analyzed using GSVA, to obtain the reference data set.

In certain embodiments, the reference data set comprises one or more enrichment scores of the reference biological samples, wherein for a respective reference biological sample one or more enrichment scores are generated based on one or more of the Tables selected from Tables 19-1 to 19-36, Tables 19A-1 to 19A-36, Table 20, Table 21, Table 22, Tables 23-1 to 23-28, Tables 25-1 to 25-32, Tables 26-1 to 26-60, Tables 27-1 to 27-48, and Table 28-1 to 28-22, wherein for each selected Table, at least one enrichment score of the respective reference biological sample based on the selected Table is generated based on enrichment of expression of at least 2 genes (or one or more human orthologs thereof) selected from the genes listed in the respective selected Table, in the respective reference biological sample. In certain embodiments, for a reference biological sample, the one or more enrichment scores of the reference biological sample can be generated using the same method as that used for the patient (test) sample (e.g., using the same selected Tables and genes selected from the selected Tables). The at least 2 genes, effective number of genes, all genes (or one or more human orthologs thereof) selected from the genes listed in a respective selected Table, can form the input gene set for generating the at least one enrichment score based on the respective selected Table. Enrichment of the input gene set formed based on a selected Table, in a reference biological sample can be measured for generating the at least one enrichment score based on the selected Table, of the reference biological sample. In certain embodiments, the one or more Tables are selected from Tables 19-1 to 19-36. In certain embodiments, the one or more Tables are selected from Tables 19A-1 to 19A-36. In certain embodiments, the one or more Tables are selected from Tables 26-1 to 26-60. In certain embodiments, the one or more Tables are selected from Tables 27-1 to 27-48. In certain embodiments, the one or more Tables are selected from Tables 23-1 to 23-28. In certain embodiments, the one or more Tables are selected from Tables 25-1 to 25-32. In certain embodiments, the one or more Tables are selected from Tables 28-1 to 28-22. The one or more Tables selected, and the genes selected from the selected Tables for generating the one or more enrichment scores of the reference biological samples can be same as the one or more Tables selected, and the genes selected from the selected Tables respectively used for generating the one or more enrichment scores of the patient, and can be any of the selected Tables and selected genes described herein. The one or more enrichment scores can comprise the at least one enrichment score from each of the selected Table. The at least 2 genes of the reference data set can include the at least 2 genes from each of the selected table. In certain embodiments, the selected tables of the data set (e.g., based on which the one or more enrichment scores of the patient are generated), and the selected tables of the reference data set (e.g., based on which the one or more enrichment scores of the reference biological samples are generated) can at least partially overlap (e. g., one or more selected Tables can be same). In certain embodiments, the selected tables of the data set, and the selected tables of the reference data set are the same. In certain embodiments, the selected tables and genes selected from the selected Tables of the data set, and the selected tables and genes selected from the selected Tables of the reference data set, are the same. Enrichment of expression the selected genes (or one or more human orthologs thereof) in a respective reference biological sample, e.g., for calculating the one or more enrichment scores of the respective reference biological sample, can be measured by comparing the gene expression from the respective reference biological sample with that of the cohort (e.g., the reference biological samples). In certain embodiments, the one or more enrichment scores of the patient are generated based on comparing the data set with a reference data set, wherein the reference data set can be a reference data set described herein. In certain embodiments, the one or more enrichment scores of the patient are generated based on comparing the data set with the reference data set, and the enrichment of expression of the selected genes, (e.g., for calculating the one or more enrichment scores of the patient) in the biological sample from the patient can be calculated based on comparing gene expression measurement data of the biological sample, with the gene expression measurement data of the reference biological samples. In certain embodiments, the machine learning model is trained based on the one or more enrichment scores of the reference biological samples, and analyzing the data set include providing the one or more enrichment scores of the patient to the trained machine learning model. The reference data set used for generating the one or more enrichment scores of the patient, can be the same as or different from the reference data set used for training the machine learning model. In certain embodiments, the reference data set used for generating the one or more enrichment scores of the patient, is same as the reference data set used for training the machine learning model. The enrichment score can be generated using any suitable method, including but not limited to GSEA, and GSVA. In certain embodiments, the enrichment scores are generated based on GSVA, and the enrichment scores are GSVA scores.

In certain embodiments, the reference data set is obtained using GSVA, wherein the reference data set comprises one or more GSVA scores of the reference biological samples, wherein for a respective reference biological sample one or more GSVA scores are generated based on one or more of the Tables selected from Tables 19-1 to 19-36, Tables 19A-1 to 19A-36, Table 20, Table 21, Table 22, Tables 23-1 to 23-28, Tables 25-1 to 25-32, Tables 26-1 to 26-60, Tables 27-1 to 27-48, and Table 28-1 to 28-22, wherein for each selected Table, at least one GSVA score of the respective reference biological sample based on the selected Table is generated based on enrichment of expression of at least 2 genes selected from the genes listed in the respective selected Table, in the respective reference biological sample. In certain embodiments, for a reference biological sample, the one or more GSVA scores of the reference biological sample can be generated using a method same (e.g., using the same selected Tables and genes selected from the selected Tables) as of the patient. The at least 2 genes, effective number of genes, all genes (or one or more human orthologs thereof) selected from the genes listed in a respective selected Table, can form the input gene set for generating the at least one GSVA score based on the respective selected Table, using GSVA. Enrichment of the input gene set formed based on a selected Table, in a reference biological sample can be measured for generating the at least one GSVA score based on the selected Table of the reference biological sample. In certain embodiments, the one or more Tables are selected from Tables 19-1 to 19-36. In certain embodiments, the one or more Tables are selected from Tables 19A-1 to 19A-36. In certain embodiments, the one or more Tables are selected from Tables 26-1 to 26-60. In certain embodiments, the one or more Tables are selected from Tables 27-1 to 27-48. In certain embodiments, the one or more Tables are selected from Tables 23-1 to 23-28. In certain embodiments, the one or more Tables are selected from Tables 25-1 to 25-32. In certain embodiments, the one or more Tables are selected from Tables 28-1 to 28-22. The one or more Tables selected, and the genes selected from the selected Tables for generating the one or more GSVA scores of the reference biological samples can be same as the one or more Tables selected, and the genes selected from the selected Tables respectively used for generating the one or more GSVA scores of the patient, and can be any of the selected Tables and selected genes described herein. The one or more GSVA scores can comprise the at least one GSVA score from each of the selected Table. The at least 2 genes of the reference data set can include the at least 2 genes from each of the selected table. In certain embodiments, the selected tables of the data set (e.g., based on which the one or more GSVA scores of the patient are generated), and the selected tables of the reference data set (e.g., based on which the one or more GSVA scores of the reference biological samples are generated) can at least partially overlap (e. g., one or more selected Tables can be same). In certain embodiments, the selected tables of the data set, and the selected tables of the reference data set are the same. In certain embodiments, the selected tables and genes selected from the selected Tables of the data set, and the selected tables and genes selected from the selected Tables of the reference data set, are the same. Enrichment of expression of the selected genes in a respective reference biological sample, e.g., for calculating the one or more GSVA scores of the respective reference biological sample, can be measured by comparing the gene expression from the respective reference biological sample with that of the cohort (e.g., the reference biological samples). In certain embodiments, the one or more GSVA scores of the patient are generated based on comparing the data set with a reference data set, wherein the reference data set can be a reference data set described herein. In certain embodiments, the one or more GSVA scores of the patient are generated based on comparing the data set with the reference data set, and the enrichment of expression of the selected genes, (e.g., for calculating the one or more GSVA scores of the patient) in the biological sample from the patient can be calculated based on comparing gene expression measurement data of the biological sample, with the gene expression measurement data of the reference biological samples. In certain embodiments, the machine learning model is trained based on the one or more GSVA scores of the reference biological samples, and analyzing the data set include providing the one or more GSVA scores of the patient to the trained machine learning model. The reference data set used for generating the one or more GSVA scores of the patient, can be same or different as the reference data set used for training the machine learning model. In certain embodiments, the reference data set used for generating the one or more GSVA scores of the patient, is same as the reference data set used for training the machine learning model. In certain embodiments, the reference data set can be a data set described in the examples. The reference subjects can be human. The patient can be a human patient.

The trained machine-learning model can be trained (e.g., obtained by training) using linear regression, logistic regression, Ridge regression, Lasso regression, elastic net (EN) regression, support vector machine (SVM), gradient boosted machine (GBM), k nearest neighbors (kNN), generalized linear model (GLM), naïve Bayes (NB) classifier, neural network, Random Forest (RF), deep learning algorithm, linear discriminant analysis (LDA), decision tree learning (DTREE), adaptive boosting (ADB), Classification and Regression Tree (CART), hierarchical clustering, or any combination thereof. The algorithm of the trained machine learning model can be a machine learning classifier, e.g., mentioned in this paragraph. The machine learning classifier (e.g., linear regression, LOG, Ridge regression, Lasso regression, EN regression, SVM, GBM, kNN, GLM, NB classifier, neural network, a RF, deep learning algorithm, LDA, DTREE, ADB, CART, and/or hierarchical clustering) can be trained to obtain the trained machine learning model. In some embodiments, the trained machine learning model, is trained using a supervised machine learning algorithm or an unsupervised machine learning algorithm, e.g., the classifier can be a supervised machine learning algorithm or an unsupervised machine learning algorithm. In certain embodiments, the trained machine-learning model is trained using linear regression. In certain embodiments, the trained machine-learning model is trained using logistic regression. In certain embodiments, the trained machine-learning model is trained using Lasso regression. In certain embodiments, the trained machine-learning model is trained using EN regression. In certain embodiments, the trained machine-learning model is trained using SVM. In certain embodiments, the trained machine-learning model is trained using GBM. In certain embodiments, the trained machine-learning model is trained using kNN. In certain embodiments, the trained machine-learning model is trained using GLM. In certain embodiments, the trained machine-learning model is trained using NB classifier. In certain embodiments, the trained machine-learning model is trained using neural network. In certain embodiments, the trained machine-learning model is trained using RF. In certain embodiments, the trained machine-learning model is trained using deep learning algorithm. In certain embodiments, the trained machine-learning model is trained using LDA. In certain embodiments, the trained machine-learning model is trained using DTREE. In certain embodiments, the trained machine-learning model is trained using ADB. In certain embodiments, the trained machine-learning model is trained using CART. In certain embodiments, the trained machine-learning model is trained using hierarchical clustering.

The LN disease state of the patient can be classified with an accuracy of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The LN disease state of the patient can be classified with a sensitivity of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The LN disease state of the patient can be classified with a specificity of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The LN disease state of the patient can be classified with a positive predictive value of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The LN disease state of the patient can be classified with a negative predictive value of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The LN disease state of the patient can be classified with a Receiver operating characteristic (ROC) curve having an Area-Under-Curve (AUC) of at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99. The trained machine learning model can have a Receiver operating characteristic (ROC) curve having an Area-Under-Curve (AUC) of at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99 for classifying LN disease states.

In some embodiments, the method classifies the LN disease state of the patient with an accuracy of 70% to 100%. In some embodiments, the method classifies the LN disease state of the patient with an accuracy of 70% to 75%, 70% to 80%, 70% to 85%, 70% to 90%, 70% to 92%, 70% to 95%, 70% to 96%, 70% to 97%, 70% to 98%, 70% to 99%, 70% to 100%, 75% to 80%, 75% to 85%, 75% to 90%, 75% to 92%, 75% to 95%, 75% to 96%, 75% to 97%, 75% to 98%, 75% to 99%, 75% to 100%, 80% to 85%, 80% to 90%, 80% to 92%, 80% to 95%, 80% to 96%, 80% to 97%, 80% to 98%, 80% to 99%, 80% to 100%, 85% to 90%, 85% to 92%, 85% to 95%, 85% to 96%, 85% to 97%, 85% to 98%, 85% to 99%, 85% to 100%, 90% to 92%, 90% to 95%, 90% to 96%, 90% to 97%, 90% to 98%, 90% to 99%, 90% to 100%, 92% to 95%, 92% to 96%, 92% to 97%, 92% to 98%, 92% to 99%, 92% to 100%, 95% to 96%, 95% to 97%, 95% to 98%, 95% to 99%, 95% to 100%, 96% to 97%, 96% to 98%, 96% to 99%, 96% to 100%, 97% to 98%, 97% to 99%, 97% to 100%, 98% to 99%, 98% to 100%, or 99% to 100%. In some embodiments, the method classifies the LN disease state of the patient with an accuracy of 70%, 75%, 80%, 85%, 90%, 92%, 95%, 96%, 97%, 98%, 99%, or 100%. In some embodiments, the method classifies the LN disease state of the patient with an accuracy of at least 70%, 75%, 80%, 85%, 90%, 92%, 95%, 96%, 97%, 98%, or 99%. In some embodiments, the method classifies the LN disease state of the patient with a sensitivity of 70% to 100%. In some embodiments, the method classifies the LN disease state of the patient with a sensitivity of 70% to 75%, 70% to 80%, 70% to 85%, 70% to 90%, 70% to 92%, 70% to 95%, 70% to 96%, 70% to 97%, 70% to 98%, 70% to 99%, 70% to 100%, 75% to 80%, 75% to 85%, 75% to 90%, 75% to 92%, 75% to 95%, 75% to 96%, 75% to 97%, 75% to 98%, 75% to 99%, 75% to 100%, 80% to 85%, 80% to 90%, 80% to 92%, 80% to 95%, 80% to 96%, 80% to 97%, 80% to 98%, 80% to 99%, 80% to 100%, 85% to 90%, 85% to 92%, 85% to 95%, 85% to 96%, 85% to 97%, 85% to 98%, 85% to 99%, 85% to 100%, 90% to 92%, 90% to 95%, 90% to 96%, 90% to 97%, 90% to 98%, 90% to 99%, 90% to 100%, 92% to 95%, 92% to 96%, 92% to 97%, 92% to 98%, 92% to 99%, 92% to 100%, 95% to 96%, 95% to 97%, 95% to 98%, 95% to 99%, 95% to 100%, 96% to 97%, 96% to 98%, 96% to 99%, 96% to 100%, 97% to 98%, 97% to 99%, 97% to 100%, 98% to 99%, 98% to 100%, or 99% to 100%. In some embodiments, the method classifies the LN disease state of the patient with a sensitivity of 70%, 75%, 80%, 85%, 90%, 92%, 95%, 96%, 97%, 98%, 99%, or 100%. In some embodiments, the method classifies the LN disease state of the patient with a sensitivity of at least 70%, 75%, 80%, 85%, 90%, 92%, 95%, 96%, 97%, 98%, or 99%. In some embodiments, the method classifies the LN disease state of the patient with a specificity of 70% to 100%. In some embodiments, the method classifies the LN disease state of the patient with a specificity of 70% to 75%, 70% to 80%, 70% to 85%, 70% to 90%, 70% to 92%, 70% to 95%, 70% to 96%, 70% to 97%, 70% to 98%, 70% to 99%, 70% to 100%, 75% to 80%, 75% to 85%, 75% to 90%, 75% to 92%, 75% to 95%, 75% to 96%, 75% to 97%, 75% to 98%, 75% to 99%, 75% to 100%, 80% to 85%, 80% to 90%, 80% to 92%, 80% to 95%, 80% to 96%, 80% to 97%, 80% to 98%, 80% to 99%, 80% to 100%, 85% to 90%, 85% to 92%, 85% to 95%, 85% to 96%, 85% to 97%, 85% to 98%, 85% to 99%, 85% to 100%, 90% to 92%, 90% to 95%, 90% to 96%, 90% to 97%, 90% to 98%, 90% to 99%, 90% to 100%, 92% to 95%, 92% to 96%, 92% to 97%, 92% to 98%, 92% to 99%, 92% to 100%, 95% to 96%, 95% to 97%, 95% to 98%, 95% to 99%, 95% to 100%, 96% to 97%, 96% to 98%, 96% to 99%, 96% to 100%, 97% to 98%, 97% to 99%, 97% to 100%, 98% to 99%, 98% to 100%, or 99% to 100%. In some embodiments, the method classifies the LN disease state of the patient with a specificity of 70%, 75%, 80%, 85%, 90%, 92%, 95%, 96%, 97%, 98%, 99%, or 100%. In some embodiments, the method classifies the LN disease state of the patient with a specificity of at least 70%, 75%, 80%, 85%, 90%, 92%, 95%, 96%, 97%, 98%, or 99%. In some embodiments, the method classifies the LN disease state of the patient with a positive predictive value of 70% to 100%. In some embodiments, the method classifies the LN disease state of the patient with a positive predictive value of 70% to 75%, 70% to 80%, 70% to 85%, 70% to 90%, 70% to 92%, 70% to 95%, 70% to 96%, 70% to 97%, 70% to 98%, 70% to 99%, 70% to 100%, 75% to 80%, 75% to 85%, 75% to 90%, 75% to 92%, 75% to 95%, 75% to 96%, 75% to 97%, 75% to 98%, 75% to 99%, 75% to 100%, 80% to 85%, 80% to 90%, 80% to 92%, 80% to 95%, 80% to 96%, 80% to 97%, 80% to 98%, 80% to 99%, 80% to 100%, 85% to 90%, 85% to 92%, 85% to 95%, 85% to 96%, 85% to 97%, 85% to 98%, 85% to 99%, 85% to 100%, 90% to 92%, 90% to 95%, 90% to 96%, 90% to 97%, 90% to 98%, 90% to 99%, 90% to 100%, 92% to 95%, 92% to 96%, 92% to 97%, 92% to 98%, 92% to 99%, 92% to 100%, 95% to 96%, 95% to 97%, 95% to 98%, 95% to 99%, 95% to 100%, 96% to 97%, 96% to 98%, 96% to 99%, 96% to 100%, 97% to 98%, 97% to 99%, 97% to 100%, 98% to 99%, 98% to 100%, or 99% to 100%. In some embodiments, the method classifies the LN disease state of the patient with a positive predictive value of 70%, 75%, 80%, 85%, 90%, 92%, 95%, 96%, 97%, 98%, 99%, or 100%. In some embodiments, the method classifies the LN disease state of the patient with a positive predictive value of at least 70%, 75%, 80%, 85%, 90%, 92%, 95%, 96%, 97%, 98%, or 99%. In some embodiments, the method classifies the LN disease state of the patient with a negative predictive value of 70% to 100%. In some embodiments, the method classifies the LN disease state of the patient with a negative predictive value of 70% to 75%, 70% to 80%, 70% to 85%, 70% to 90%, 70% to 92%, 70% to 95%, 70% to 96%, 70% to 97%, 70% to 98%, 70% to 99%, 70% to 100%, 75% to 80%, 75% to 85%, 75% to 90%, 75% to 92%, 75% to 95%, 75% to 96%, 75% to 97%, 75% to 98%, 75% to 99%, 75% to 100%, 80% to 85%, 80% to 90%, 80% to 92%, 80% to 95%, 80% to 96%, 80% to 97%, 80% to 98%, 80% to 99%, 80% to 100%, 85% to 90%, 85% to 92%, 85% to 95%, 85% to 96%, 85% to 97%, 85% to 98%, 85% to 99%, 85% to 100%, 90% to 92%, 90% to 95%, 90% to 96%, 90% to 97%, 90% to 98%, 90% to 99%, 90% to 100%, 92% to 95%, 92% to 96%, 92% to 97%, 92% to 98%, 92% to 99%, 92% to 100%, 95% to 96%, 95% to 97%, 95% to 98%, 95% to 99%, 95% to 100%, 96% to 97%, 96% to 98%, 96% to 99%, 96% to 100%, 97% to 98%, 97% to 99%, 97% to 100%, 98% to 99%, 98% to 100%, or 99% to 100%. In some embodiments, the method classifies the LN disease state of the patient with a negative predictive value of 70%, 75%, 80%, 85%, 90%, 92%, 95%, 96%, 97%, 98%, 99%, or 100%. In some embodiments, the method classifies the LN disease state of the patient with a negative predictive value of at least 70%, 75%, 80%, 85%, 90%, 92%, 95%, 96%, 97%, 98%, or 99%. In some embodiments, the AUC of the ROC curve of the trained machine learning model is 0.7 to 1, for classifying LN disease states. In some embodiments, the AUC of the ROC curve of the trained machine learning model is 0.7 to 0.75, 0.7 to 0.8, 0.7 to 0.85, 0.7 to 0.9, 0.7 to 0.92, 0.7 to 0.95, 0.7 to 0.96, 0.7 to 0.97, 0.7 to 0.98, 0.7 to 0.99, 0.7 to 1, 0.75 to 0.8, 0.75 to 0.85, 0.75 to 0.9, 0.75 to 0.92, 0.75 to 0.95, 0.75 to 0.96, 0.75 to 0.97, 0.75 to 0.98, 0.75 to 0.99, 0.75 to 1, 0.8 to 0.85, 0.8 to 0.9, 0.8 to 0.92, 0.8 to 0.95, 0.8 to 0.96, 0.8 to 0.97, 0.8 to 0.98, 0.8 to 0.99, 0.8 to 1, 0.85 to 0.9, 0.85 to 0.92, 0.85 to 0.95, 0.85 to 0.96, 0.85 to 0.97, 0.85 to 0.98, 0.85 to 0.99, 0.85 to 1, 0.9 to 0.92, 0.9 to 0.95, 0.9 to 0.96, 0.9 to 0.97, 0.9 to 0.98, 0.9 to 0.99, 0.9 to 1, 0.92 to 0.95, 0.92 to 0.96, 0.92 to 0.97, 0.92 to 0.98, 0.92 to 0.99, 0.92 to 1, 0.95 to 0.96, 0.95 to 0.97, 0.95 to 0.98, 0.95 to 0.99, 0.95 to 1, 0.96 to 0.97, 0.96 to 0.98, 0.96 to 0.99, 0.96 to 1, 0.97 to 0.98, 0.97 to 0.99, 0.97 to 1, 0.98 to 0.99, 0.98 to 1, or 0.99 to 1, for classifying LN disease states. In some embodiments, the AUC of the ROC curve of the trained machine learning model is 0.7, 0.75, 0.8, 0.85, 0.9, 0.92, 0.95, 0.96, 0.97, 0.98, 0.99, or 1, for classifying LN disease states. In some embodiments, the AUC of the ROC curve of the trained machine learning model is at least 0.7, 0.75, 0.8, 0.85, 0.9, 0.92, 0.95, 0.96, 0.97, 0.98, or 0.99, for classifying LN disease states.

The trained machine-learning model can have the accuracy, sensitivity, specificity, positive predictive value, negative predictive value, and ROC-AUC value, described above, and the accuracy, sensitivity, specificity, positive predictive value, negative predictive value, and ROC-AUC value of the method for classifying the LN disease state of the patient can be based on the classification parameters (e.g., accuracy, sensitivity, specificity, positive predictive value, negative predictive value, and ROC-AUC respectively) of the trained machine-learning model for classifying the LN disease state of patients, as described herein and/or as understood by one of skill in the art. In certain embodiments, the accuracy, sensitivity, specificity, positive predictive value, and negative predictive value for classifying LN disease state of the patient can be the accuracy, sensitivity, specificity, positive predictive value, and negative predictive value respectively for classifying whether the patient has acute LN, transitional LN, or chronic LN, or does not have LN. In certain embodiments, the accuracy, sensitivity, specificity, positive predictive value, and negative predictive value for classifying LN disease state of the patient can be the accuracy, sensitivity, specificity, positive predictive value, and negative predictive value respectively for classifying whether the patient has acute LN, transitional LN, or chronic LN. In certain embodiments, the accuracy, sensitivity, specificity, positive predictive value, and negative predictive value for classifying LN disease state of the patient can be the accuracy, sensitivity, specificity, positive predictive value, and negative predictive value respectively for classifying whether the patient has acute LN, transitional LN, chronic group I LN, or chronic group II LN, or does not have LN. In certain embodiments, the accuracy, sensitivity, specificity, positive predictive value, and negative predictive value for classifying LN disease state of the patient can be the accuracy, sensitivity, specificity, positive predictive value, and negative predictive value respectively for classifying whether the patient has acute LN, transitional LN, chronic group I LN, or chronic group II LN. In certain embodiments, the accuracy, sensitivity, specificity, positive predictive value, and negative predictive value for classifying LN disease state of the patient can be the accuracy, sensitivity, specificity, positive predictive value, and negative predictive value respectively for classifying whether the patient has LN. The accuracy, sensitivity, specificity, positive predictive value, and/or negative predictive value can be calculated based on the AUC of the ROC curve of the trained machine learning model for classifying the LN disease states.

In certain embodiments, the LN disease state of the patient is classified based on a LN disease risk score. The LN disease risk score can be generated from the data set. In certain embodiments, the LN disease risk score is generated based on the one or more GSVA scores of the patient. In certain embodiments, the LN disease state of the patient is classified based on comparing the LN disease risk score of the patient to one or more reference values. In certain embodiments, generating the LN disease risk score of the patient comprises developing one or more weighted GSVA scores of the patient from the one or more GSVA scores, and summing the one or more weighted GSVA scores to obtain the disease risk score of the patient. For a respective GSVA score of the one or more GSVA scores, the corresponding weighted GSVA score is obtained by multiplying the respective GSVA score with its corresponding weight factor, wherein the corresponding weight factor is determined based on contribution of the set of genes based on which the respective GSVA score is generated, on the classification of the LN disease state of the patient. The set of genes based on which the respective GSVA score is generated are the genes based on enrichment of expression which in the biological sample, the respective GSVA score is generated. In certain particular embodiments, the one or more GSVA scores of the patient is binarized, and the binarized GSVA scores are multiplied with the corresponding weight factors to obtain the weighted GSVA scores. In certain embodiments, binarizing the one or more GSVA scores includes replacing all GSVA scores (e.g., of the one or more GSVA scores) above a threshold value with a first value, and replacing all GSVA scores (e.g., of the one or more GSVA scores) equal to or below the threshold value with a second value. In certain particular embodiments, the threshold value is 0, the first value is 1, and the second value is 0. The one or more GSVA scores can be generated using a method as described herein. In certain embodiments, the weight factors are calculated based on training a machine learning model, wherein the trained machine learning model can generate an inference indicating the LN disease state of a reference subject based on the one or more GSVA scores of the reference subject. The trained machine learning model can be a trained machine learning model as described herein, and/or can be trained according a method and a reference data set as described herein. The gene sets based on which the one or more GSVA scores are generated can be features of the machine learning model. The GSVA scores can be the feature values. For a respective reference subject GSVA score generated based on a gene set, can be the feature value of the gene set for the respective reference subject. The feature co-efficients of the features can be the weight factors. The corresponding weight factor for a respective GSVA score is the feature co-efficient of the gene set (e.g., a feature) based on which the GSVA score is generated. The feature co-efficient, can be the average feature co-efficients of the iterations run during training the model. In certain embodiments, the machine learning model is trained using the one or more GSVA scores of the reference subjects (e.g., of a reference dataset described herein) and a ridge regression algorithm with penalty, to obtain the weight factors. In certain embodiments, the machine learning model was trained using the one or more GSVA scores of the reference subjects (e.g., of a reference dataset described herein) having acute LN disease state and of the reference subjects having group II chronic LN disease state, and a ridge regression algorithm with penalty, to obtain the weight factors.

In certain embodiments, classifying the LN disease state of the patient includes classifying whether the patient has acute LN disease state, transitional LN disease state, or chronic LN disease state, or does not have LN, e.g., the LN disease state of the patient is classified as acute lupus nephritis, transitional lupus nephritis, chronic lupus nephritis, or absence of lupus nephritis. In certain embodiments, classifying the LN disease state of the patient includes classifying whether the patient has acute LN disease state, transitional LN disease state, or chronic LN disease state. In certain embodiments, classifying the LN disease state of the patient includes classifying whether the patient has acute LN disease state, transitional LN disease state, chronic group I LN disease state, or chronic group II LN disease state, or does not have LN. In certain embodiments, classifying the LN disease state of the patient includes classifying whether the patient has acute LN disease state, transitional LN disease state, chronic group I LN disease state, or chronic group II LN disease state. In certain embodiments, classifying LN disease state of the patient includes classifying whether the patient has LN. In certain embodiments, the LN disease state of the patient is classified as acute LN disease state, transitional LN disease state, chronic LN disease state, or absence of LN. In certain embodiments, the LN disease state of the patient is classified as acute LN disease state, transitional LN disease state, or chronic LN disease state. In certain embodiments, the LN disease state of the patient is classified as acute LN disease state, transitional LN disease state, chronic group I LN disease state, chronic group II LN disease state, or absence of LN. In certain embodiments, the LN disease state of the patient is classified as acute LN disease state, transitional LN disease state, chronic group I LN disease state, or chronic group II LN disease state. In certain embodiments, the LN disease state of the patient is classified as presence of LN, or absence of LN. The chronic LN disease state can be chronic group I LN disease state or chronic group II LN disease state.

The inference of the trained machine learning model can include a confidence value between 0 and 1. In certain embodiments, the confidence value of the inference of the trained machine learning model is between 0 and 1, such as, 0, 0.1, 0.2, 0.3, 0.4, 0.5, 0.6, 0.7, 0.8, 0.9 or 1, or any value or ranges there between, that the patient has acute LN. In certain embodiments, the confidence value of the inference of the trained machine learning model is between 0 and 1, such as, 0, 0.1, 0.2, 0.3, 0.4, 0.5, 0.6, 0.7, 0.8, 0.9 or 1, or any value or ranges there between, that the patient has transitional LN. In certain embodiments, the confidence value of the inference of the trained machine learning model is between 0 and 1, such as, 0, 0.1, 0.2, 0.3, 0.4, 0.5, 0.6, 0.7, 0.8, 0.9 or 1, or any value or ranges there between, that the patient has chronic LN. In certain embodiments, the confidence value of the inference of the trained machine learning model is between 0 and 1, such as, 0, 0.1, 0.2, 0.3, 0.4, 0.5, 0.6, 0.7, 0.8, 0.9 or 1, or any value or ranges there between, that the patient has chronic group I LN disease state. In certain embodiments, the confidence value of the inference of the trained machine learning model is between 0 and 1, such as, 0, 0.1, 0.2, 0.3, 0.4, 0.5, 0.6, 0.7, 0.8, 0.9 or 1, or any value or ranges there between, that the patient has chronic group II LN disease state.

In certain embodiments, the patient has lupus. In certain embodiments, the patient has LN. In certain embodiments, the patient is at elevated risk of having lupus. In certain embodiments, the patient is at elevated risk of having LN. In certain embodiments, the patient is suspected of having lupus. In certain embodiment, the patient is suspected of having LN. In certain embodiments, the patient is asymptomatic for lupus. In certain embodiments, the patient is asymptomatic for LN.

In certain embodiments, the method further includes recommending, selecting and/or administering a treatment to the patient based at least in part on the classification of the LN disease state of the patient. In certain embodiments, the method further includes administering a treatment to the patient based at least in part on the classification of the LN disease state of the patient. In certain embodiments, the treatment is configured to treat LN. In certain embodiments, the treatment is configured to reduce a severity of LN. In certain embodiments, the treatment is configured to reduce a risk of having LN. In certain embodiments, the treatment administered is configured to prevent, reverse and/or slow disease progression from non-disease state to acute LN, acute LN to transitional LN, and/or transitional LN to chronic LN. In certain embodiments, the treatment administered is configured to prevent, reverse and/or slow disease progression from non-disease state to acute LN. In certain embodiments, the treatment administered is configured to prevent, reverse and/or slow disease progression from acute LN to transitional LN. In certain embodiments, the treatment administered is configured to prevent, reverse and/or slow disease progression from transitional LN to chronic LN. In certain embodiments, the treatment administered is configured to prevent, reverse and/or slow disease progression from acute LN to transitional LN, and/or transitional LN to chronic LN. In certain embodiments, the treatment configured to prevent, reverse and/or slow disease progression from acute LN to transitional LN, may be configured to target inflammatory macrophages, and/or the GC/Tfh cell response. In certain embodiments, the treatment configured to prevent, reverse and/or slow disease progression from transitional LN to chronic LN, may be configured to protect kidney tubules. In certain embodiments, the treatment configured to protect kidney tubules may target mitochondrial dysfunction in the kidney tubulointerstitial tissue. In certain embodiments, a patient determined to have acute LN is administered with the treatment configured to prevent, reverse and/or slow disease progression from acute LN to transitional LN. In certain embodiments, a patient determined to have transitional LN is administered with the treatment configured to prevent, reverse and/or slow disease progression from transitional LN to chronic LN, and/or the treatment configured to prevent, reverse and/or slow disease progression from acute LN to transitional LN. The treatment can include one or more treatments of lupus nephritis. The treatment can comprises a pharmaceutical composition. As shown in Example 3, increased enrichment of inflammatory cells/pathways at the transitional LN stage appears to represent whether mice will progress to chronic LN stage and/or renal failure. Relatively early detection of unique gene signatures indicative of transitional LN, can be beneficial in treating LN, and stop disease progression to chronic LN stage and/or renal failure. Specific features of this stage can include an enrichment of gene signatures for IFN, Th17 cells, and/or increased enrichment of macrophages, and the treatment administered can include one or more pharmaceutical composition targeting one or more molecular pathways associated with these gene signatures. In certain embodiments, the one or more pharmaceutical composition targeting one or more molecular pathways associated with enrichment of gene signatures for IFN, Th17 cells, and/or increased enrichment of macrophages, can include one or more of anifrolumab, secukinumab, ibrutinib, or the like. In certain embodiments, the treatment administered can include the one or more pharmaceutical composition targeting one or more molecular pathways associated with enrichment of gene signatures for IFN, Th17 cells, and/or increased enrichment of macrophages. In certain embodiments, the treatment administered can include one or more of anifrolumab, secukinumab, ibrutinib, or the like. In certain embodiments, a patient determined to have transitional LN is administered with the treatment comprising the one or more pharmaceutical composition targeting one or more molecular pathways associated with enrichment of gene signatures for IFN, Th17 cells, and/or increased enrichment of macrophages. In certain embodiments, the treatment comprises a neutrophil function inhibitor, a TNF inhibitor, an IL1 inhibitor, a Plasma cell inhibitor, a NK cell inhibitor, a B Cell Inhibitor, an IFN inhibitor, or any combination thereof. Non-limiting examples of an IFN inhibitor include Anifrolumab. Non-limiting examples of a Plasma cell inhibitor include Mycophenolate, Bortezomib, Carfilzomib, Ixazomib, Daratumumab, Isatuximab and Elotuzumab. Mycophenolate can be Mycophenolate Mofetil. Non-limiting examples of an IL1 inhibitor include Anakinra, and Canakinumab. Non-limiting examples of a TNF inhibitor include Adalimumab, Certolizumab pegol, Etanercept, Golimumab, and Infliximab. Non-limiting examples of a Neutrophil function inhibitor include Dasatinib, Apremilast, and Roflumilast. Non-limiting examples of a NK cell inhibitor include Azathioprine. Non-limiting examples of a B cell inhibitor include Belimumab, Rituximab, Obinutuzumab, Ocrelizumab, Ofatumumab, and Inebilizumab. In certain embodiments, the treatment comprises Anifrolumab, Mycophenolate, Bortezomib, Carfilzomib, Ixazomib, Daratumumab, Isatuximab, Elotuzumab, Anakinra, Canakinumab Adalimumab, Certolizumab pegol, Etanercept, Golimumab, Infliximab, Dasatinib, Apremilast, Roflumilast, Azathioprine, Belimumab, Rituximab, Obinutuzumab, Ocrelizumab, Ofatumumab, Inebilizumab, or any combination thereof. In certain embodiments, the treatment for acute LN disease state comprises a neutrophil function inhibitor, a TNF inhibitor, an IL1 inhibitor, a Plasma cell inhibitor, a NK cell inhibitor, a B Cell Inhibitor, an IFN inhibitor, or any combination thereof. In certain embodiments, the treatment for acute LN disease state comprises Anifrolumab, Mycophenolate, Bortezomib, Carfilzomib, Ixazomib, Daratumumab, Isatuximab, Elotuzumab, Anakinra, Canakinumab Adalimumab, Certolizumab pegol, Etanercept, Golimumab, Infliximab, Dasatinib, Apremilast, Roflumilast, Azathioprine, Belimumab, Rituximab, Obinutuzumab, Ocrelizumab, Ofatumumab, Inebilizumab, or any combination thereof. In certain embodiments, a patient determined to have transitional LN is administered with the treatment comprising an IFN inhibitor. In certain embodiments, a patient determined to have transitional LN is administered with the treatment comprising an IFN inhibitor, a Th 17 cell inhibitor, a B cell inhibitor, or any combination thereof. In certain embodiments, the treatment for transitional LN disease state comprises an IFN inhibitor, a TNF inhibitor, a Th 17 cell inhibitor, a B cell inhibitor, or any combination thereof. In certain embodiments, a patient determined to have transitional LN is administered with the treatment comprising anifrolumab, secukinumab, ibrutinib, or the like. In certain embodiments, the treatment for transitional LN disease state comprises anifrolumab, secukinumab, ibrutinib, or the like. In certain embodiments, the treatment for transitional LN disease state comprises anifrolumab, secukinumab, ibrutinib, belimumab, Rituximab, Obinutuzumab, Ocrelizumab, Ofatumumab, Inebilizumab, or any combination thereof. In certain embodiments, the treatment for transitional LN disease state comprises anifrolumab, secukinumab, ibrutinib, belimumab, Rituximab, Obinutuzumab, Ocrelizumab, Ofatumumab, Inebilizumab, Adalimumab, Certolizumab pegol, Etanercept, Golimumab, and Infliximab or any combination thereof. In certain embodiments, the treatment for chronic LN disease state comprises kidney transplantation. Association between mitochondrial dysfunction and loss of kidney tubule cells indicates that attempts to target immunometabolism by treatment with anti-diabetic drugs such as metformin may exacerbate metabolic defects and contribute to kidney damage in lupus nephritis patients. In certain embodiments, the treatment administered does not target immunometabolism by treatment with anti-diabetic drugs such as metformin. In certain embodiments, the treatment administered does not include an anti-diabetic drug, such as metformin, that targets immunometabolism. In certain embodiments, the treatment administered to a patient determined to have transitional LN, does not include an anti-diabetic drug, such as metformin, that targets immunometabolism.

The biological sample can comprise a kidney biopsy sample, a blood sample, isolated peripheral blood mononuclear cells (PBMCs), urine sample, or any derivative thereof. In certain embodiments, the biological sample comprises a kidney biopsy sample or any derivative thereof. In certain embodiments, the biological sample comprises a blood sample or any derivative thereof. In certain embodiments, the biological sample comprises PBMCs or any derivative thereof. In certain embodiments, the biological sample comprises a urine sample or any derivative thereof. The reference biological samples can comprise kidney biopsy samples, blood samples, isolated peripheral blood mononuclear cells (PBMCs), urine samples, or any derivative thereof. In certain embodiments, the reference biological samples comprise kidney biopsy samples or any derivative thereof. In certain embodiments, the reference biological samples comprise blood samples or any derivative thereof. In certain embodiments, the reference biological samples comprise PBMCs or any derivative thereof. In certain embodiments, the reference biological samples comprise urine samples or any derivative thereof. The biological sample and the reference biological sample can be of similar type. In certain embodiments, the biological sample comprises PBMCs or any derivative thereof, and the reference biological samples comprise PBMCs or any derivative thereof. In certain embodiments, the biological sample comprises a blood sample or any derivative thereof, and the reference biological samples comprise blood samples or any derivative thereof. In certain embodiments, the biological sample comprises a kidney biopsy sample or any derivative thereof, and the reference biological samples comprise kidney biopsy samples or any derivative thereof. The blood sample can be whole blood sample or any derivative thereof. In certain embodiments, the kidney biopsy sample contains renal cortex. In certain embodiments, the kidney biopsy sample contains non-microdissected renal cortex. In certain embodiments, the kidney biopsy sample contains glomerular tissue. In certain embodiments, the kidney biopsy sample contains tubulointerstitial tissue. In certain embodiments, the kidney biopsy sample contains glomerular and tubulointerstitial tissue. In certain embodiments, the biological sample comprise a blood sample, or any derivative thereof, and the data set comprises and/or derived from gene expression measurement of genes selected from the genes listed in Tables 25-1 to 25-32, e.g., the one or more Tables are selected from Tables 25-1 to 25-32. In certain embodiments, the biological sample comprise PBMCs, or any derivative thereof, and the data set comprises and/or derived from gene expression measurement of genes selected from the genes listed in Tables 25-1 to 25-32, e.g., the one or more Tables are selected from Tables 25-1 to 25-32. In certain embodiments, the biological sample comprise a kidney biopsy sample, or any derivative thereof, and the data set comprises and/or derived from gene expression measurement of genes selected from the genes listed in Tables 23-1 to 23-28, e.g., the one or more Tables are selected from Tables 23-1 to 23-28. In certain embodiments, the biological sample comprise a kidney biopsy sample, or any derivative thereof, and the data set comprises and/or derived from gene expression measurement of the genes selected from genes listed in Tables 28-1 to 28-22, e.g., the one or more Tables are selected from Tables 28-1 to 28-22. In certain embodiments, the biological sample comprise a kidney biopsy sample, or any derivative thereof, wherein the kidney biopsy sample contains glomerular tissue and the data set comprises and/or derived from gene expression measurement of genes selected from genes listed in Tables 28-1 to 28-8, and 28-12 to 28-22, e.g., the one or more Tables selected comprise Tables 28-1 to 28-8, and 28-12 to 28-22. In certain embodiments, the biological sample comprise a kidney biopsy sample, or any derivative thereof, wherein the kidney biopsy sample contains tubulointerstitial tissue and the data set comprises and/or derived from gene expression measurement of genes selected from genes listed in Tables 28-1 to 28-4, 28-6 to 28-18, and 28-20 to 28-22, e.g., the one or more Tables selected comprise Tables 28-1 to 28-4, 28-6 to 28-18, and 28-20 to 28-22. In certain embodiments, the biological sample comprise a kidney biopsy sample, or any derivative thereof, wherein the kidney biopsy sample contains tubulointerstitial tissue and the data set comprises and/or derived from gene expression measurement of genes selected from genes listed in Tables 28-1 to 28-18, and 28-20 to 28-22, e.g., the one or more Tables selected comprise Tables 28-1 to 28-18, and 28-20 to 28-22. The patient can be a human patient.

To obtain a kidney biopsy sample, various techniques may be used. The kidney biopsy sample can include kidney samples removed from the body. Kidney biopsy can be performed using any suitable technique known to those of skill in the art. In certain embodiments, kidney biopsy can be performed using percutaneous (through the skin) biopsy, open biopsy, or any combination thereof. Percutaneous biopsy can include isolating the kidney biopsy sample by inserting a needle through the skin that lies above the kidney. Open biopsy can include isolating the kidney biopsy sample during surgery. The area, size, and amount of the kidney biopsy sample may vary depending upon the condition being analyzed.

In certain embodiments, the method further comprises monitoring the LN disease state of the patient, wherein the monitoring comprises assessing the LN disease state of the patient at a plurality of different time points. A difference in the assessment of the LN disease state of the patient among the plurality of time points can be indicative of one or more clinical indications selected from the group consisting of: (i) a diagnosis of the LN disease state of the patient, (ii) a prognosis of the LN disease state of the patient, and (iii) an efficacy or non-efficacy of a course of treatment for treating the LN disease state of the patient. In certain embodiments, the patient has been administered a treatment, and the method can assess an efficacy or non-efficacy of the treatment, for treating the LN disease state of the patient.

Acute, transitional and chronic lupus nephritis disease state can be characterized by gene enrichment analysis corresponding to the coral group (acute LN), yellow group (transitional LN), purple group (chronic group I LN) and black group (chronic group II LN) as described in Example 4, and FIGS. 74A and 74B (in a kidney/renal biopsy sample), and/or in FIG. 77 (in a blood sample).

Patients having acute lupus nephritis disease state can have transcriptomic characteristics corresponding to (e.g., falls within) the coral group, as described in Example 4, FIGS. 74A and 74B (in a kidney/renal biopsy sample), and/or FIG. 77 (in a blood sample). In certain embodiments, patients having acute lupus nephritis disease state have i) unchanged expression, of one or more immune/inflammatory cell signature gene modules, ii) minimally decreased expression of one or more kidney cell signature gene modules, iii) unchanged expression of one or more metabolic signature gene modules, iv) unchanged expression of one or more endothelial cell signature gene modules, and/or v) unchanged expression of one or more fibroblast signature gene modules, in a kidney biopsy sample compared to a control. The control can be subjects without LN. In certain embodiments, patients with acute lupus nephritis disease state have i) a mean GSVA score of 0.33±0.1 of kidney cell signature gene modules, ii) a mean GSVA score of −0.12±0.1 of endothelial cell signature gene modules, iii) a mean GSVA score of −0.15±0.1 of fibroblast signature gene modules, iv) a mean GSVA score of −0.15±0.1 of mesangial cell signature gene modules, v) a mean GSVA score of 0.24±0.1 of metabolic signatures gene modules, vi) a mean GSVA score of −0.26±0.1 of immune/inflammatory cell signature gene modules, or any combination thereof, where the GSVA scores are determined with the method, and with respect to the data set described in example 4. In certain embodiments, disease pathology of patients having acute LN is sufficiently confined to glomeruli, with or no or minimum damage to kidney cells, and kidney tubules. In certain embodiments, glomeruli of patients having acute LN is increased in size compared to a control. In certain embodiments, glomeruli of patients having acute LN have immune complex deposition. In certain embodiments, glomeruli of patients having acute LN are increased in size with immune cell infiltration and/or immune complex deposition, such as IgG, C3, and/or anti-nuclear antibody (ANA) deposits. Patients having acute lupus nephritis disease state can have any one or more characteristics selected from those described in this paragraph.

Patients having transitional LN disease state, have more severe disease compared to patients with acute LN, but less severe disease compared to chronic LN. In certain embodiments, patients having transitional LN disease state have transcriptomic characteristics corresponding to (e.g., falls within) the yellow group, as described in Example 4, FIGS. 74A and 74B (in a kidney/renal biopsy sample), and/or FIG. 77 (in a blood sample). In certain embodiments, patients having transitional LN disease state have i) minimally increased expression of one or more immune/inflammatory cell signature gene modules, ii) minimally decreased expression of one or more kidney cell signature gene modules, iii) minimally decreased expression of one or more metabolic signature gene modules, iv) minimally increased expression of one or more endothelial cell signature gene modules, and/or v) minimally increased expression of one or more fibroblast signature gene modules, in a kidney biopsy sample compared to a control. The control can be subjects without LN. In certain embodiments, patients having transitional lupus nephritis disease state have i) a mean GSVA score of 0.18±0.1 of kidney cell signature gene modules, ii) a mean GSVA score of 0.02±0.1 of endothelial cell signature gene modules, iii) a mean GSVA score of −0.02±0.1 of fibroblast signature gene modules, iv) a mean GSVA score of −0.06±0.1 of mesangial cell signature gene modules, v) a mean GSVA score of 0.17±0.1 of metabolic signatures gene modules, vi) a mean GSVA score of 0.05±0.1 of immune/inflammatory cell signature gene modules, or any combination thereof, where the GSVA scores are determined with the method, and with respect to the data set described in example 4. In certain embodiments, patients having transitional LN have inflammatory disease, with no or minimal kidney cell damage and/or no or minimal metabolic dysfunction. In certain embodiments, glomeruli of patients having transitional LN have immune cell infiltration with IgG and/or C3 deposition. For patients having transitional LN, IgG and/or C3 deposition in glomeruli, and serum levels of anti-DNA antibodies can be higher compared to acute LN stage. Interstitium of patients having transitional LN can have more inflammatory cells than acute LN stage, and tubular cells may show some dilation and atrophy. Patients having transitional LN may have minimum to no tubule damage. Patients having transitional lupus nephritis disease state can have any one or more characteristics selected from those described in this paragraph.

Patients having chronic LN disease state, have more severe disease compared to patients with transitional LN. In certain embodiments, patients having chronic LN disease state have transcriptomic characteristics corresponding to (e.g., falls within) the purple group (chronic group I LN) or black group (chronic group II LN), as described in Example 4, FIGS. 74A and 74B (in a kidney/renal biopsy sample), and/or FIG. 77 (in a blood sample). In certain embodiments, patients having chronic group I LN disease state have i) increased expression of one or more immune/inflammatory cell signature gene modules, ii) decreased expression of one or more kidney cell signature gene modules, iii) decreased expression of one or more metabolic signature gene modules, iv) unchanged expression of one or more endothelial cell signature gene modules, and/or v) increased expression of one or more fibroblast signature gene modules, in a kidney biopsy sample compared to a control. The control can be subjects without LN. In certain embodiments, chronic group I LN disease state have i) a mean GSVA score of −0.24±0.1 of kidney cell signature gene modules, ii) a mean GSVA score of −0.07±0.1 of endothelial cell signature gene modules, iii) a mean GSVA score of 0.08±0.1 of fibroblast signature gene modules, iv) a mean GSVA score of 0.09±0.1 of mesangial cell signature gene modules, v) a mean GSVA score of −0.19±0.1 of metabolic signatures gene modules, vi) a mean GSVA score of 0.31±0.1 of immune/inflammatory cell signature gene modules, or any combination thereof, where the GSVA scores are determined with the method, and with respect to the data set described in example 4. In certain embodiments, patients having chronic group I LN disease state have inflammatory disease with kidney cell damage and/or metabolic dysfunction. In certain embodiments, patients having chronic group II LN disease state have i) unchanged expression of one or more immune/inflammatory cell signature gene modules, ii) decreased expression of one or more kidney cell signature gene modules, iii) decreased expression of one or more metabolic signature gene modules, iv) increased expression of one or more endothelial cell signature gene modules, and/or v) increased expression of one or more fibroblast signature gene modules, in a kidney biopsy sample compared to a control. The control can be subjects without LN. In certain embodiments, chronic group II LN disease state have i) a mean GSVA score of −0.21±0.1 of kidney cell signature gene modules, ii) a mean GSVA score of 0.25±0.1 of endothelial cell signature gene modules, iii) a mean GSVA score of 0.07±0.1 of fibroblast signature gene modules, iv) a mean GSVA score of 0.1±0.1 of mesangial cell signature gene modules, v) a mean GSVA score of −0.15±0.1 of metabolic signatures gene modules, vi) a mean GSVA score of −0.25±0.1 of immune/inflammatory cell signature gene modules, or any combination thereof, where the GSVA scores are determined with the method, and with respect to the data set described in example 4. In certain embodiments, patients having chronic group II LN disease state have kidney cell damage and/or metabolic dysfunction, with no or minimal inflammation. In certain embodiments, patients having chronic LN, exhibit glomerular sclerosis, fibrosis with interstitial inflammation, and elevated level of immune complex depositions. Immune complex depositions in patients having chronic LN can be higher compared to acute and transitional disease stages. In certain embodiments, 80% or more tubular cells of patients with chronic LN, have tubular dilation, and/or with evidence of atrophy and tubular casts. Patients having chronic lupus nephritis disease state can have any one or more characteristics selected from those described in this paragraph.

The gene modules are disclosed in Table 23-1 to 23-28, and Tables 28-1 to 28-22. Immune/inflammatory cell signature gene modules can include Anergic/activated T cell, B Cell signature module, Dendritic Cell signature module, GC B Cell signature module, Granulocyte signature module, LDG signature module, Monocyte/Myeloid Cell signature module, NK Cell signature module, PDC signature module, Plasma Cell signature module, Platelet signature module, Antigen presenting cell signature module, CD8 T cell signature module, IG Chain cell signature module, Monocyte-Macrophage signature module, Myeloid Cell signature module, Platelet signature module, Tfh Cell signature module, Th17 Cell signature module, and T Cell signature module. Metabolic signature gene modules can include Amino Acid Metabolism signature module, Fatty Acid Oxidation signature module, Fatty Acid Alpha Oxidation signature module, Fatty Acid Beta Oxidation signature module, Glycolysis signature module, Oxidative Phosphorylation signature module, Pentose Phosphate signature module, general mitochondria signature module, and TCA cycle signature module. Kidney cell signature gene modules can include Collecting Duct signature module, Distal Tubule signature module, Kidney Cell signature module, Loop of Henle signature module, Mesangial Cell signature module, Podocyte signature module, and Proximal Tubule signature module.

Unchanged expression of a gene module in a patient, compared to a control can refer to GSVA score change, e.g., GSVA score of the module for the patient vs. GSVA score of the module for the control, of less than ±0.1 (e.g., within −0.1 and +0.1). Minimally increased expression of a gene module, compared to control can refer to GSVA score change of 0 to ≤0.2. Minimally decreased expression of a gene module, compared to control can refer to GSVA score change of −0.2≤to 0. Increased expression of a gene module, compared to control can refer to GSVA score change of greater than 0.2. Decrease expression of a gene module, compared to control can refer to GSVA can refer to GSVA score change more negative than −0.2. The control can be subjects/patients with absence of lupus nephritis. The GSVA scores (discussed in this paragraph) can be determined with the method, and with respect to the data set described herein, e.g., in example 4.

Certain aspects are directed to use of a data set described herein.

Certain aspects of the present disclosure is directed to a method for validating a mouse model useful for identifying and/or characterizing a human disease. The method can include (a) providing a gene set capable of classifying a mouse as having an endotype selected from two or more endotypes of the disease; (b) determining human orthologs of the gene set; (c) classifying a human patient as having an endotype selected from the two or more endotypes of the disease using the human orthologs; and/or (d) using the human orthologs to classify the mouse model as having an endotype selected from the two or more endotypes of the disease. The endotype of a validated mouse model classified using the human orthologs can correspond to the human endotype of step (c) identified using the human orthologs. The disease can be lupus, or lupus nephritis. In certain embodiments, the disease is lupus nephritis. In certain embodiments, disease is lupus nephritis, and the endotypes of lupus nephritis are acute LN, transitional LN, and chronic LN.

In another aspect, the present disclosure provides a method of identifying one or more records having a specific phenotype, the method comprising: receiving a plurality of first records, wherein each first record is associated with one or more of a plurality of phenotypes; receiving a plurality of second records, wherein each second record is associated with one or more of the plurality of phenotypes, and wherein the plurality of second records and the plurality of first records are non-overlapping; applying a machine learning algorithm to at least one first record and at least one second record to determine a classifier; receiving a plurality of third records, wherein the third records are distinct from the plurality of first records and the plurality of second records; and applying the classifier to the plurality of third records to identify one or more third records associated with the specific phenotype.

In some embodiments, the first records and the second records comprise nucleic acid sequencing data, transcriptome data, genome data, epigenome data, proteome data, metabolome data, virome data, methylome data, lipidomic data, lineage-ome data, nucleosomal occupancy data, a genetic variant, a gene fusion, an insertion or deletion (indel), or any combination thereof. In some embodiments, the first records and the second records are in different formats. In some embodiments, the first records and the second records are from different sources, different studies, or both. In some embodiments, the phenotype comprises a disease state, an organ involvement, a medication response, or any combination thereof. In some embodiments, the classifier comprises an elastic generalized linear model classifier, a k-nearest neighbors classifier, a random forest classifier, or any combination thereof.

In some embodiments, the elastic generalized linear model classifier employs an elastic penalty of about 0.8 to about 1. In some embodiments, the elastic generalized linear model classifier employs an elastic penalty of at least about 0.8, about 0.825, about 0.85, about 0.875, about 0.9, about 0.925, about 0.95, about 0.975, or about 1. In some embodiments, the elastic generalized linear model classifier employs an elastic penalty of at most about 0.8, about 0.825, about 0.85, about 0.875, about 0.9, about 0.925, about 0.95, about 0.975, or about 1. In some embodiments, the elastic generalized linear model classifier employs an elastic penalty of about 0.8 to about 0.825, about 0.8 to about 0.85, about 0.8 to about 0.875, about 0.8 to about 0.9, about 0.8 to about 0.925, about 0.8 to about 0.95, about 0.8 to about 0.975, about 0.8 to about 1, about 0.825 to about 0.85, about 0.825 to about 0.875, about 0.825 to about 0.9, about 0.825 to about 0.925, about 0.825 to about 0.95, about 0.825 to about 0.975, about 0.825 to about 1, about 0.85 to about 0.875, about 0.85 to about 0.9, about 0.85 to about 0.925, about 0.85 to about 0.95, about 0.85 to about 0.975, about 0.85 to about 1, about 0.875 to about 0.9, about 0.875 to about 0.925, about 0.875 to about 0.95, about 0.875 to about 0.975, about 0.875 to about 1, about 0.9 to about 0.925, about 0.9 to about 0.95, about 0.9 to about 0.975, about 0.9 to about 1, about 0.925 to about 0.95, about 0.925 to about 0.975, about 0.925 to about 1, about 0.95 to about 0.975, about 0.95 to about 1, or about 0.975 to about 1. In some embodiments, the elastic generalized linear model classifier employs an elastic penalty of about 0.8, about 0.825, about 0.85, about 0.875, about 0.9, about 0.925, about 0.95, about 0.975, or about 1.

In some embodiments, the k-nearest neighbors classifier employs a K value of the size of the plurality of distinct first data sets, wherein k is about 1 to about 20. In some embodiments, the k-nearest neighbors classifier employs a K value of the size of the plurality of distinct first data sets, wherein k is at least about 1, about 2, about 3, about 4, about 5, about 6, about 8, about 10, about 12, about 14, about 16, or about 20. In some embodiments, the k-nearest neighbors classifier employs a K value of the size of the plurality of distinct first data sets, wherein k is at most about 1, about 2, about 3, about 4, about 5, about 6, about 8, about 10, about 12, about 14, about 16, or about 20. In some embodiments, the k-nearest neighbors classifier employs a K value of the size of the plurality of distinct first data sets, wherein k is about 1 to about 2, about 1 to about 3, about 1 to about 4, about 1 to about 5, about 1 to about 6, about 1 to about 8, about 1 to about 10, about 1 to about 12, about 1 to about 14, about 1 to about 16, about 1 to about 20, about 2 to about 3, about 2 to about 4, about 2 to about 5, about 2 to about 6, about 2 to about 8, about 2 to about 10, about 2 to about 12, about 2 to about 14, about 2 to about 16, about 2 to about 20, about 3 to about 4, about 3 to about 5, about 3 to about 6, about 3 to about 8, about 3 to about 10, about 3 to about 12, about 3 to about 14, about 3 to about 16, about 3 to about 20, about 4 to about 5, about 4 to about 6, about 4 to about 8, about 4 to about 10, about 4 to about 12, about 4 to about 14, about 4 to about 16, about 4 to about 20, about 5 to about 6, about 5 to about 8, about 5 to about 10, about 5 to about 12, about 5 to about 14, about 5 to about 16, about 5 to about 20, about 6 to about 8, about 6 to about 10, about 6 to about 12, about 6 to about 14, about 6 to about 16, about 6 to about 20, about 8 to about 10, about 8 to about 12, about 8 to about 14, about 8 to about 16, about 8 to about 20, about 10 to about 12, about 10 to about 14, about 10 to about 16, about 10 to about 20, about 12 to about 14, about 12 to about 16, about 12 to about 20, about 14 to about 16, about 14 to about 20, or about 16 to about 20. In some embodiments, the k-nearest neighbors classifier employs a K value of the size of the plurality of distinct first data sets, wherein k is about 1, about 2, about 3, about 4, about 5, about 6, about 8, about 10, about 12, about 14, about 16, or about 20.

In some embodiments, the K-value of the random forest classifier is incremented by 1 if the k-value is an even number. In some embodiments, applying a machine learning algorithm to the third data set comprises applying a machine learning algorithm to a plurality of unique third data sets.

In some embodiments, the classifier identifies said one or more third records associated with the specific phenotype with an accuracy of about 70% to about 100%. In some embodiments, the classifier identifies said one or more third records associated with the specific phenotype with an accuracy of at least about 70%, about 75%, about 80%, about 85%, about 90%, about 95%, or about 100%. In some embodiments, the classifier identifies said one or more third records associated with the specific phenotype with an accuracy of at most about 70%, about 75%, about 80%, about 85%, about 90%, about 95%, or about 100%. In some embodiments, the classifier identifies said one or more third records associated with the specific phenotype with an accuracy of about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the classifier identifies said one or more third records associated with the specific phenotype with an accuracy of about 70%, about 75%, about 80%, about 85%, about 90%, about 95%, or about 100%.

In some embodiments, the classifier identifies said one or more third records associated with the specific phenotype with an accuracy of about 70% to about 100%. In some embodiments, the classifier identifies said one or more third records associated with the specific phenotype with an accuracy of at least about 70%, about 75%, about 80%, about 85%, about 90%, about 95%, or about 100%. In some embodiments, the classifier identifies said one or more third records associated with the specific phenotype with an accuracy of at most about 70%, about 75%, about 80%, about 85%, about 90%, about 95%, or about 100%. In some embodiments, the classifier identifies said one or more third records associated with the specific phenotype with an accuracy of about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the classifier identifies said one or more third records associated with the specific phenotype with an accuracy of about 70%, about 75%, about 80%, about 85%, about 90%, about 95%, or about 100%.

In some embodiments, the classifier herein enables a specific phenotype association sensitivity of about 70% to about 100%. In some embodiments, the classifier herein enables a specific phenotype association sensitivity of at least 70%, about 75%, about 80%, about 85%, about 90%, about 95%, or about 100%. In some embodiments, the classifier herein enables a specific phenotype association sensitivity of at most 70%, about 75%, about 80%, about 85%, about 90%, about 95%, or about 100%. In some embodiments, the classifier herein enables a specific phenotype association sensitivity of about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the classifier herein enables a specific phenotype association sensitivity of about 70%, about 75%, about 80%, about 85%, about 90%, about 95%, or about 100%.

In some embodiments, the classifier herein enables a specific phenotype association specificity of about 70% to about 100%. In some embodiments, the classifier herein enables a specific phenotype association specificity of at least 70%, about 75%, about 80%, about 85%, about 90%, about 95%, or about 100%. In some embodiments, the classifier herein enables a specific phenotype association specificity of at most 70%, about 75%, about 80%, about 85%, about 90%, about 95%, or about 100%. In some embodiments, the classifier herein enables a specific phenotype association specificity of about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the classifier herein enables a specific phenotype association specificity of about 70%, about 75%, about 80%, about 85%, about 90%, about 95%, or about 100%.

In some embodiments, the method further comprises filtering the first records, the second records, or both. In some embodiments, the filtering comprises removing outliers, removing background noise, removing data without annotation data, normalizing, scaling, variance correcting, Weighted Gene Co-expression Network Analysis, enrichment analysis, dimensionality reduction, or any combination thereof. In some embodiments, the normalizing is performed by Robust Multi-Array Analysis (RMA), Guanine Cytosine Robust Multi-Array Analysis (GCRMA), Linear Models for Microarray Data, variance stabilizing transformation (VST), normal-exponential quantile correction (NEQC), or any combination thereof. In some embodiments, the variance correction comprises employing a local empirical Bayesian shrinkage, adjusting the p-values for multiple hypothesis testing using the Benjamini-Hochberg correction, and removing all data with a set false discovery rate

In some embodiments, the false discovery rate is about 0.000001 to about 0.2. In some embodiments, the false discovery rate is at least about 0.000001. In some embodiments, the false discovery rate is at most about 0.2. In some embodiments, the false discovery rate is about 0.000001 to about 0.00005, about 0.000001 to about 0.00001, about 0.000001 to about 0.0005, about 0.000001 to about 0.0001, about 0.000001 to about 0.005, about 0.000001 to about 0.001, about 0.000001 to about 0.05, about 0.000001 to about 0.01, about 0.000001 to about 0.2, about 0.00005 to about 0.00001, about 0.00005 to about 0.0005, about 0.00005 to about 0.0001, about 0.00005 to about 0.005, about 0.00005 to about 0.001, about 0.00005 to about 0.05, about 0.00005 to about 0.01, about 0.00005 to about 0.2, about 0.00001 to about 0.0005, about 0.00001 to about 0.0001, about 0.00001 to about 0.005, about 0.00001 to about 0.001, about 0.00001 to about 0.05, about 0.00001 to about 0.01, about 0.00001 to about 0.2, about 0.0005 to about 0.0001, about 0.0005 to about 0.005, about 0.0005 to about 0.001, about 0.0005 to about 0.05, about 0.0005 to about 0.01, about 0.0005 to about 0.2, about 0.0001 to about 0.005, about 0.0001 to about 0.001, about 0.0001 to about 0.05, about 0.0001 to about 0.01, about 0.0001 to about 0.2, about 0.005 to about 0.001, about 0.005 to about 0.05, about 0.005 to about 0.01, about 0.005 to about 0.2, about 0.001 to about 0.05, about 0.001 to about 0.01, about 0.001 to about 0.2, about 0.05 to about 0.01, about 0.05 to about 0.2, or about 0.01 to about 0.2. In some embodiments, the false discovery rate is about 0.000001, about 0.00005, about 0.00001, about 0.0005, about 0.0001, about 0.005, about 0.001, about 0.05, about 0.01, or about 0.2.

In some embodiments, the Weighted Gene Co-expression Network Analysis comprises calculating a topology matrix, clustering the data based on the topology matrix, and correlating module eigenvalues for traits on a linear scale by Pearson correlation, for nonparametric traits by Spearman correlation, and for dichotomous traits by point-biserial correlation or t-test. The Pearson correlation or the Product Moment Correlation Coefficient (PMCC), is a number between −1 and 1 that indicates the extent to which two variables are linearly related. The Spearman correlation is a nonparametric measure of rank correlation; statistical dependence between the rankings of two variables.

In another aspect, the present disclosure provides a non-transitory computer-readable storage media encoded with a computer program including instructions executable by a processor to create an application for identifying one or more records having a specific phenotype, the application comprising: a first receiving module receiving a plurality of first records, wherein each first record is associated with one or more of a plurality of phenotypes; a second receiving module receiving a plurality of second records, wherein each second record is associated with one or more of the plurality of phenotypes, and wherein the plurality of second records and the plurality of first records are non-overlapping; a machine learning module applying a machine learning algorithm to at least one first record and at least one second record to determine a classifier; a third receiving module receiving a plurality of third records, wherein the third records are distinct from the plurality of first records and the plurality of second records; and a classifying module applying the classifier to the plurality of third records to identify one or more third records associated with the specific phenotype.

In some embodiments, the first records and the second records comprise nucleic acid sequencing data, transcriptome data, genome data, epigenome data, proteome data, metabolome data, virome data, methylome data, lipidomic data, lineage-ome data, nucleosomal occupancy data, a genetic variant, a gene fusion, an insertion or deletion (indel), or any combination thereof. In some embodiments, the first records and the second records are in different formats. In some embodiments, the first records and the second records are from different sources, different studies, or both. In some embodiments, the phenotype comprises a disease state, an organ involvement, a medication response, or any combination thereof. In some embodiments, the classifier comprises an elastic generalized linear model classifier, a k-nearest neighbors classifier, a random forest classifier, or any combination thereof. In some embodiments, the elastic generalized linear model classifier employs an elastic penalty of about 0.9. In some embodiments, the k-nearest neighbors classifier employs a K-value of about 5% of the size of the plurality of distinct first data sets. In some embodiments, the K-value of the random forest classifier is incremented by 1 if the k-value is an even number. In some embodiments, applying a machine learning algorithm to the third data set comprises applying a machine learning algorithm to a plurality of unique third data sets. In some embodiments, said classifier identifies said one or more third records associated with the specific phenotype with an accuracy of at least about 70%. In some embodiments, the method further comprises filtering the first records, the second records, or both. In some embodiments, the filtering comprises removing outliers, removing background noise, removing data without annotation data, normalizing, scaling, variance correcting, Weighted Gene Co-expression Network Analysis, enrichment analysis, dimensionality reduction, or any combination thereof. In some embodiments, the normalizing is performed by Robust Multi-Array Analysis (RMA), Guanine Cytosine Robust Multi-Array Analysis (GCRMA), Linear Models for Microarray Data, variance stabilizing transformation (VST), normal-exponential quantile correction (NEQC), or any combination thereof. In some embodiments, the variance correction comprises employing a local empirical Bayesian shrinkage, adjusting the p-values for multiple hypothesis testing using the Benjamini-Hochberg correction, and removing all data with a false discovery rate of less than 0.2. In some embodiments, the Weighted Gene Co-expression Network Analysis comprises calculating a topology matrix, clustering the data based on the topology matrix, and correlating module eigenvalues for traits on a linear scale by Pearson correlation, for nonparametric traits by Spearman correlation, and for dichotomous traits by point-biserial correlation or t-test.

In some embodiments, the plurality of quantitative measures comprises gene expression measurements. In some embodiments, the immunological state comprises an active or inactive state of each of one or more of the plurality of genomic loci. In some embodiments, the plurality of genomic loci comprises one or more genes selected from the group consisting of: RAB4B, ADAR, MRPL44, CDCA5, MYD88, SNN, BRD3, C7orf43, CDC20, SP1, POFUT1, SAMD4B, ATP6V1B2, TSPAN9, SP140, STK26, IRF4, LCP1, LMO2, SF3B4, HIST2H2AA3, CITED4, ADAM8, TICAM1, and HSD17B7.

### Biological Data Analysis

In another aspect, the present disclosure provides a computer-implemented method for assessing a condition of a subject, comprising: (a) receiving a dataset of a biological sample of the subject; (b) selecting one or more data analysis tools, wherein the one or more data analysis tools comprise an analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool, or a combination thereof; (c) processing the dataset using the one or more data analysis tools to generate a data signature of the biological sample of the subject; and (d) based at least in part on the data signature generated in (c), assessing the condition of the subject. For use in the context of the methods set forth in the present disclosure, any tools and methods known to those in the skill of the art may be applied, e.g., as described in “Machine Learning Disease Prediction and Treatment Prioritization,” published as U.S. Pat. App. Pub. No. 2021/0104321 (and WO 2020/102043), incorporated herein by reference in its entirety.

In some embodiments, the dataset comprises mRNA gene expression or transcriptome data, DNA genomic data, proteomic data, metabolomic data, or a combination thereof. In some embodiments, the biological sample comprises a whole blood (WB) sample, a PBMC sample, a tissue sample, a cell sample, or any derivative thereof. In some embodiments, assessing the condition of the subject comprises identifying a disease or disorder of the subject.

In some embodiments, the method further comprises identifying a disease or disorder of the subject at a sensitivity or specificity of at least about 70%. In some embodiments, the method further comprises determining a likelihood of the identification of the disease or disorder of the subject. In some embodiments, the method further comprises providing a therapeutic intervention for the disease or disorder of the subject. In some embodiments, the method further comprises monitoring the disease or disorder of the subject, wherein the monitoring comprises assessing the disease or disorder of the subject at a plurality of time points, wherein the assessing is based at least on the disease or disorder identified at each of the plurality of time points.

In some embodiments, selecting the one or more data analysis tools comprises receiving a user selection of the one or more data analysis tools. In some embodiments, selecting the one or more data analysis tools is automatically performed by the computer without receiving a user selection of the one or more data analysis tools.

In another aspect, the present disclosure provides a computer system for assessing a condition of a subject, comprising: a database that is configured to store a dataset of a biological sample of the subject; and one or more computer processors operatively coupled to the database, wherein the one or more computer processors are individually or collectively programmed to: (i) select one or more data analysis tools, wherein the one or more data analysis tools comprise an analysis tool selected from the group consisting of: a BIG-C™ big data analysis tool, an I_Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool; (ii) process the dataset using the one or more data analysis tools to generate a data signature of the biological sample of the subject; and (iii) based at least in part on the data signature generated in (ii), assess the condition of the subject.

In another aspect, the present disclosure provides a non-transitory computer readable medium comprising machine-executable code that, upon execution by one or more computer processors, implements a method for assessing a condition of a subject, the method comprising: (a) receiving a dataset of a biological sample of the subject; (b) selecting one or more data analysis tools wherein the one or more data analysis tools comprise an analysis tool selected from the group consisting of: a BIG-C™ big data analysis tool, an I_Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool; (c) processing the dataset using the one or more data analysis tools to generate a data signature of the biological sample of the subject; and (d) based at least in part on the data signature generated in (c), assessing the condition of the subject. In any embodiment described herein, the one or more data analysis tools may be a plurality of data analysis tools each independently selected from a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool.

In an aspect, the present disclosure provides systems and methods for using bioinformatics approaches to deconvolute bulk mRNA for various cells and processes involved in lupus, psoriasis, atopic dermatitis, and/or systemic sclerosis (scleroderma) organ pathology, including inflammatory cells, endothelial cells, tissue cells.

In an aspect, the present disclosure provides systems and methods for the delineation of the altered metabolism of cells by using gene expression analysis.

In an aspect, the present disclosure provides systems and methods for using various regression models (e.g., classification and regression trees, linear regression, step-wise regression) to dissect the specific metabolic alterations in individual cell types.

In an aspect, the present disclosure provides systems and methods for using animal models and the ability to translate mouse gene expression into the human equivalent to confirm the results in humans and also analyze the effects of treatment.

In an aspect, the present disclosure provides systems and methods for the delineation of the role of specific cells (myeloid cells) and processes (interferon, mitochondrial dysfunction) in lupus, psoriasis, atopic dermatitis, and/or systemic sclerosis (scleroderma) tissue pathology.

In an aspect, the present disclosure provides systems and methods for using non-lymphocyte populations in skin and kidney toward diagnostic and/or prognostic biopsy tests.

In an aspect, the present disclosure provides systems and methods for defining gene signatures in individual cell types in a mixed population such as blood or tissue (e.g., skin, kidney).

In an aspect, the present disclosure provides systems and methods for analyzing sets of metabolism genes and their relationship to function and cell type, including subsets of myeloid cells.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, including any supplementary materials, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent application file contains at least one drawing executed in color. Copies of this patent application publication with color drawings(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1I show that dysregulation of metabolic gene signatures is common among lupus-affected tissues. FIG. 1A: Comparison of DEGs among DLE, class III/IV LN GL, and class III/IV LN TI (lupus nephritis tubulointerstitial inflammation). FIG. 1B: MCODE protein-protein interactions of common UP and DOWN DEGs were generated with Cytoscape using the STRING and ClusterMaker2 plugins and annotated with BIG-C functional categories (odds ratio (OR)>1, p<0.05) in Adobe Illustrator. Overlap p-value was calculated using Fisher's exact test. GSVA of signatures for glycolysis (FIG. 1C), the PPP (FIG. 1D), the TCA cycle (FIG. 1E), OXPHOS (FIG. 1F), FAAO (FIG. 1G), FABO (FIG. 1H), and AA metabolism (FIG. 11) in lupus tissues and controls (CTLs; unshaded plot on left in each comparison).

FIGS. 2A-2C show that increased myeloid cell signatures and decreased non-hematopoietic cell signatures characterize the majority of lupus patients. FIG. 2A: Hedges' g effect sizes of immune and non-hematopoietic cell signatures in DLE, class III/IV LN GL, and class III/IV LN TI as compared to tissue CTLs. Significant p-values reflect significant differences in enrichment of the immune cell signatures or non-hematopoietic cell signatures in lupus tissues as compared to CTL as determined by Welch's t-test with Bonferroni correction (FIG. 9). FIG. 2B: R² values derived from linear regression of the monocyte-derived macrophage or the tissue-resident macrophage markers with the monocyte/MC GSVA scores in individual patients and CTLs from lupus-affected tissues (FIGS. 11A-11B). Significant p-values reflect significantly non-zero slopes. FIG. 2C: Pearson correlation coefficients between tissue-resident macrophage markers in LN.

FIGS. 3A-3U show that metabolic and cellular signature changes in class II LN GL are similar to those seen in class III/IV. GSVA of metabolic pathway signatures (FIGS. 3A-3G) and cell signatures (FIGS. 3H-3T) in all classes of LN GL. Each point represents an individual sample. Significant differences in enrichment of the metabolic signatures, immune cell signatures, or non-hematopoietic cell signatures between class II LN GL and CTL, class III/IV LN GL and CTL, and class II LN GL and class III/IV LN GL were performed by Welch's t-test with Bonferroni correction. FIG. 3U: Hierarchical clustering (k=4) of all glomerular samples.

FIGS. 4A-4H show that metabolic gene expression changes in LN GL are associated with changes in the EC, kidney cell, and fibroblast gene signatures. FIG. 4A: Stepwise regression coefficients and FIGS. 4B-4H CART analysis for metabolic pathway signatures in all glomerular LN samples and CTLs.

FIGS. 5A-5O show that mitochondrial and peroxisomal signature changes and local hypoxia contribute to changes in metabolic gene expression in specific cells. GSVA of signatures for mitochondria- (FIGS. 5A-5F) or peroxisome-related gene signatures (FIGS. 5G-5H) in lupus tissues and CTLs. Each point represents an individual sample. FIGS. 5I-5K: Stepwise regression coefficients for mitochondrial and peroxisomal signatures in all tissues and CTLs. FIG. 5L: GSVA of HIF1A in lupus tissues and CTLs. Each point represents an individual sample. FIGS. 5M-5O: Stepwise regression coefficients for metabolic pathway signatures with the addition of HIF1A in all tissues and CTLs.

FIGS. 6A-6H show that metabolic gene expression changes occur independent of acute IFN stimulation in murine LN. FIG. 6A: GSVA of the IGS in the kidney of IFNα-accelerated NZB/W mice (GSE86423). FIGS. 6B-6H: GSVA of metabolic signatures and linear regression between the IGS and metabolic signature GSVA scores.

FIGS. 7A-7E show that metabolic gene expression changes in murine LN are corrected with immunosuppressive treatment. GSVA of metabolism signatures in the kidney of NZM2410 (GSE32583, GSE49898) (FIG. 7A), NZB/W (GSE32583, GSE49898) (FIG. 7B), IFNα-accelerated NZB/W (GSE72410) (FIG. 7C), MRL/lpr (GSE153021) (FIG. 7D), and NZW/BXSB (GSE32583, GSE49898) mice (FIG. 7E) with and without treatment.

FIGS. 8A-8F show that cellular and metabolic gene expression changes correlate with expression of genes indicating tubular damage in human and murine LN. Log 2 expression of HAVCR1 Havcr1 (FIG. 8A) and LCN2 Lcn2 (FIG. 8B) in human LN TI and the kidneys of (NZM2410 (GSE32583, GSE49898), NZB/W (GSE32583, GSE49898), IFNα-accelerated NZB/W (GSE86423), IFNα-accelerated NZB/W (GSE72410), and MRL/lpr (GSE153021) mice.

FIGS. 9A-90 show that increased myeloid cell signatures and decreased tissue cell signatures characterize the majority of lupus patients. GSVA of signatures for granulocytes (FIG. 9A), pDCs (FIG. 9B), dendritic cells (FIG. 9C), monocyte/MCs (FIG. 9D), T cells (FIG. 9E), B cells (FIG. 9F), plasma cells (FIG. 9G), platelets (FIG. 9H), immune cells (FIG. 91) with expression found only in DLE, endothelial cells (FIG. 9J), fibroblasts (FIG. 9K), skin cells (FIG. 9L), kidney cells (FIG. 9M), glomerular cells (FIG. 9N), and tubule cells (FIG. 90) in lupus tissues and CTLs.

FIG. 10 shows that anergic/Activated T cell marker genes have no change in expression in LN class III/V. Log2 expression of CD160, CD244, CTLA4, ICOS, KLRG1, LAG3, and PDCD1 in lupus tissues and CTLs.

FIGS. 11A-11B show that monocyte/MC gene signatures reflect both monocyte-derived macrophage and tissue-resident macrophage populations. Linear regression between the monocyte/MC GSVA score and FCN1 expression (FIG. 11A) or TRM marker expression (FIG. 11B) in lupus-affected tissues.

FIG. 12 shows that metabolic and cellular gene expression changes in class II LN GL are similar to those seen in class III/IV. Hierarchical clustering (k=4) of class II LN GL samples (n=8).

FIGS. 13A-13U show that metabolic and cellular gene expression changes in class II LN TI are less robust than those seen in class III/IV. GSVA of metabolic pathway signatures (FIGS. 13A-13G) and cell signatures (FIGS. 13H-13T) in all classes of LN TI. Each point represents an individual sample. Significant differences in enrichment of the metabolic signatures, immune cell signatures, or non-hematopoietic cell signatures between class II LN TI and CTL, class III/IV LN TI and CTL, and class II LN TI and class III/IV LN TI were performed by Welch's t-test with Bonferroni correction. FIG. 13U: Hierarchical clustering (k=4) of all tubulointerstitial samples.

FIG. 14 shows that metabolic and cellular gene expression changes in some class II LN TI patients are similar to those seen in class III/IV patients. Hierarchical clustering (k=4) of class II LN TI samples (n=8). Each of FIGS. 13A-13T shows, from left to right: CTL points; class II LN TI points; and class II/IV LN TI points.

FIGS. 15A-15B show that numerous cellular gene signatures contribute to the observed metabolic changes in DLE. FIG. 15A: Stepwise regression coefficients for metabolic pathway GSVA scores in all samples for DLE and CTLs. For stepwise repression the pDC, skin-specific DC, monocyte/MC, T Cell, anergic/activated T cell, B cell, and plasma cell signatures were combined into the “inflammatory cell” signature because of collinearity. FIG. 15B: Hierarchical clustering (k=2) of all skin samples.

FIGS. 16A-16H show that metabolic gene expression changes in LN TI are associated with changes in the kidney cell, proximal tubule, and monocyte/MC gene signatures. Stepwise regression coefficients (FIG. 16A) and CART (FIGS. 16B-16H) analysis for metabolic pathway signatures in all tubulointerstitial LN samples and CTLs.

FIG. 17 shows that metabolic genes are altered in scRNA-seq from LN biopsies. DEGs related to metabolism in scRNA-seq clusters (CM2 left panel: tissue-resident macrophages, CT0a, center panel: effector memory CD4+ T cells, and CEO, right panel: epithelial cells) that were present in both LN patients and CTL samples from Arazi et al (Ref. 30).

FIGS. 18A-18Q show that cellular gene expression changes in NZM2410 kidneys may be corrected with immunosuppressive treatment. GSVA of immune (FIGS. 18A-18H) and non-hematopoietic (FIGS. 18I-18Q) cell signatures in the kidneys of NZM2410 mice (GSE32583, GSE49898) with and without treatment.

FIGS. 19A-19R show that cellular gene expression changes in NZB/W kidneys may be corrected with immunosuppressive treatment. GSVA of immune (FIGS. 19A-19I) and non-hematopoietic (FIGS. 19J-19R) cell signatures in the kidneys of NZB/W mice (GSE32583, GSE49898) with and without treatment. From left to right in each graph: plot 1—

FIGS. 20A-20S show that immune/inflammatory cell gene expression is increased and proximal tubule cell gene expression is decreased in IFNα-accelerated NZB/W kidneys. GSVA of immune (FIGS. 20A-20J) and non-hematopoietic (FIGS. 20K-20S) cell signatures in the kidneys of IFNα-accelerated NZB/W mice (GSE86423).

FIGS. 21A-21S show that cellular gene expression changes in IFNα-accelerated NZB/W kidneys may be corrected with immunosuppressive treatment. GSVA of immune (FIGS. 21A-21J) and non-hematopoietic (FIGS. 21K-21S) cell signatures in the kidneys of IFNα-accelerated NZB/W mice (GSE72410) with and without treatment.

FIGS. 22A-22R show that cellular gene expression in the MRL/lpr kidney is not significantly altered. GSVA of immune (FIGS. 22A-22I) and non-hematopoietic (FIGS. 22J-22R) cell signatures in the kidneys of MRL/lpr mice (GSE153021) with and without treatment.

FIGS. 23A-23Q show that immune/inflammatory cell gene expression is increased and kidney cell and proximal tubule cell gene expression is decreased in NZW/BXSB kidneys. GSVA of immune (FIGS. 23A-23H) and non-hematopoietic (FIGS. 23I-23Q) cell signatures in the kidneys of NZW/BXSB mice (GSE32583, GSE49898).

FIGS. 24A-24F show that cellular gene expression changes in murine LN correlate with metabolic gene signatures. Pearson correlation coefficients for all metabolic pathway and cellular GSVA scores in all samples of each murine LN model NZM2410 (GSE32583, GSE49898) (FIG. 24A), NZB/W (GSE32583, GSE49898) (FIG. 24B), IFNα-accelerated NZB/W (GSE86423) (FIG. 24C), IFNα-accelerated (GSE72410) (FIG. 24D), MRL/lpr (GSE153021) (FIG. 24E), and NZW/BXSB (GSE32583, GSE49898) (FIG. 24F).

FIG. 25 shows that cellular and metabolic gene expression changes correlate with expression of genes indicating tubular damage in murine LN. Correlation between Havcr1 or Lcn2 gene expression and GSVA scores for kidney cell, proximal tubule, and TCA cycle in all samples from the kidneys of NZM2410 (GSE32583, GSE49898), NZB/W (GSE32583, GSE49898), IFNα-accelerated NZB/W (GSE86423), IFNα-accelerated NZB/W (GSE72410), MRL/lpr (GSE153021), and NZW/BXSB (GSE32583) mice.

FIGS. 26A-26F show alteration/dysregulation of metabolic gene signatures in lupus, psoriasis, atopic dermatitis, and scleroderma-affected tissues. Each graph shows comparison of DEGs among class III/IV LN GL (violin plot 2), class III/IV LN TI (violin plot 4), DLE (violin plot 6), PSO (violin plot 8), AD (violin plot 10), and SSc (violin plot 12), and respective controls (unshaded violin plots 1, 3, 5, 7, 9 and 11 in each panel). The graphs show GSVA of signatures for glycolysis (FIG. 26A), the PPP (FIG. 26B), the TCA cycle (FIG. 26C), OXPHOS (FIG. 26D), FABO (FIG. 26E), and AA metabolism in lupus tissues and controls (CTLs) (FIG. 26F). Each point represents an individual sample. Numbers below each tissue indicate the number of lupus patients with enrichment scores 1 SD less than (<1SD) or greater than (>1SD) the CTL mean. Significant p-values reflect significant differences in GSVA enrichment of the metabolic or cellular signatures in each lupus tissue as compared to CTL in was determined by Welch's t-test with Bonferroni correction. **, p<0.01; ***, p<0.001; ****, p<0.0001. See methods described in relation to FIGS. 1A-1I, Example 1.

FIGS. 27A and 27B show that increased immune cell signatures and decreased non-hematopoietic cell signatures characterize the majority of lupus patients. FIG. 27A: Hedges' g effect sizes of immune cell signatures in class III/IV LN GL, class III/IV LN TI, DLE, PSO, AD, and SSc as compared to tissue CTLs. FIG. 27B: Hedges' g effect sizes of non-hematopoietic cell signatures in class III/IV LN GL, class III/IV LN TI, DLE, PSO, AD, and SSc as compared to tissue CTLs. Significant p-values reflect significant differences in GSVA enrichment of the metabolic or cellular signatures in each lupus tissue as compared to CTL was determined by Welch's t-test with Bonferroni correction. **, p<0.01; ***, p<0.001; ****, p<0.0001. See methods described in relation to FIG. 2A, Example 1.

FIGS. 28A-28C show that metabolic and cellular gene signatures are concurrently altered in the tissues of inflammatory skin diseases, with different metabolic changes reflecting different cellular signatures. Stepwise regression coefficients are shown for the glycolysis (FIG. 28A), TCA cycle (FIG. 28B), and FABO (FIG. 28C) signatures in class II-IV LN GL, class II-IV LN TI, DLE, PSO, AD, SSc and tissue CTLs. Significant p-values reflect significant coefficients in the stepwise regression model. *, p<0.05; **, p<0.01; ***, p<0.001; ****, p<0.0001. See methods described in relation to FIGS. 15A and 16A, Example 1.

FIGS. 29A-29B show DLE is characterized by enrichment of inflammatory cell and cytokine signatures, including the IFN, IL-12, and TNF signatures. FIG. 29A: Hierarchical clustering (k=4 clusters) of DLE and healthy control samples from five lupus datasets using GSVA enrichment scores of cellular and pathway gene signatures. FIG. 29B: Hedges' g effect sizes of cellular (left) and pathway (right) gene signatures for DLE compared to healthy control samples in five lupus datasets. Heatmap visualization uses red (enriched signature, >0) and blue (decreased signature, <0). Welch's t-test: *p<0.05; **p<0.01; ***p<0.001; ****p<0.0001.

FIGS. 30A-30K show enrichment of myeloid, lymphoid, IFN, IL-12, IL-23, and TNF signatures is shared among DLE, PSO, AD, and SSc. FIG. 30A: Hedges' g effect sizes of cellular (left) and pathway (right) gene signatures for disease samples compared to their respective control samples in five DLE, three PSO, two AD and three SSc datasets. Heatmap visualization uses red (enriched signature, >0) and blue (decreased signature, <0). Welch's t-test: *p<0.05; **p<0.01; ***p<0.001; ****p<0.0001. CART analysis for disease or control classification using GSVA enrichment scores in lesional: lesional DLE (FIG. 30B), lesional PSO (FIG. 30C), lesional AD (FIG. 30D) and lesional SSc (FIG. 30E), and non-lesional (NL) DLE (FIG. 30F), NL PSO (FIG. 30G), and NL AD (FIG. 30H). FIGS. 30I-K CART of nonlesional skin that was pooled without z-score normalization and non-lesional (NL) DLE (FIG. 30I), NL PSO (FIG. 30J), and NL AD (FIG. 30K). Sample numbers below bottom leaves represent the number of samples of each group classified into that leaf.

FIGS. 31A-31B show that analysis of cellular and molecular pathway signatures in lesional DLE shows increased expression of inflammatory pathways regulated by, e.g., monocytes, B cells, T cells and plasmacytoid dendritic cells (pDC). GSVA enrichment scores (y-axis) of (FIG. 31A) cellular gene signatures and (FIG. 31B) pathway gene signatures in five datasets including DLE samples and control samples. The number of DLE samples per dataset that lie −1 standard deviation of the average of the control samples is denoted on the first subtext line. The number of DLE samples per dataset that lie+1 standard deviation of the average of the control samples is denoted on the second subtext line. Welch's T-test: *p<0.05; **p<0.01; ***p<0.001; ****p<0.0001, as indicated where observed for a given pair of violin plots by a bracket and corresponding number of asterisks above the plots. Plots for DLE samples are shown in dark gray (the right plot of each pair of violin plots). Plots for control samples (CTL) are shown in light gray (the left plot of each pair of violin plots). In each panel, each pair of violin plots corresponds to analysis of NCBI Gene Expression Omnibus dataset, from left to right, GSE52471, GSE72535, GSE81071, GSE81071(2), and GSE109248. Dotted horizontal line indicates GSVA enrichment score of 0, with positive scores above and negative scores below. In FIG. 31A the panels show GSVA scores for cell types, in each row from left to right: row 1 (top row)—granulocyte, neutrophil, LDG, skin-specific DC, Langerhans; row 2 —pDC, monocyte, monocyte/myeloid, NK cell, T cell; row 3—B cell, GC B cell, plasma cell, platelet, erythrocyte; row 4: endothelial cell, fibroblast, keratinocyte, melanocyte. In FIG. 31B the panels show GSVA scores for pathways, in each row from left to right: row 1 (top row)—IFN, IL-1 cytokines, IL-12 complex, T cell IL-12 signature, IL-12, IL-17 complex; row 2—IL-21 complex, IL-23 complex, T cell IL-23 signature, TGFB fibroblast, TNF, Th17; row 3—anti-inflammation, complement proteins, inflammasome, ROS production, apoptosis, cell cycle; row 4—immunoproteasome, proteasome, unfolded protein, glycolysis, pentose phosphate, TCA cycle; row 5—OXPHOS, FAAO, FABO, AA metabolism, peroxisome.

FIGS. 32A-32B show that analysis of cellular and molecular pathway signatures in lesional PSO shows increased expression of keratinocyte cell signatures as well as TNF and Th17 pathway gene signatures. GSVA enrichment scores (y-axis) of (FIG. 32A) cellular gene signatures and (FIG. 32B) pathway gene signatures in three datasets including PSO samples and control samples. The number of PSO samples per dataset that lie −1 standard deviation of the average of the control samples is denoted on the first subtext line. The number of PSO samples per dataset that lie+1 standard deviation of the average of the control samples is denoted on the second subtext line. Welch's T-test: *p<0.05; **p<0.01; ***p<0.001; ****p<0.0001, as indicated where observed for a given pair of violin plots by a bracket and corresponding number of asterisks above the plots. Plots for PSO samples are the right plot of each pair of violin plots. Plots for control samples (CTL) are the left plot of each pair of violin plots. In each panel, each pair of violin plots corresponds to analysis of NCBI Gene Expression Omnibus dataset, from left to right, GSE52471, GSE109248, and GSE121212. Dotted horizontal line indicates GSVA enrichment score of 0, with positive scores above and negative scores below. In FIG. 32A the panels show GSVA scores for cell types, in each row from left to right: row 1 (top row)—granulocyte, neutrophil, LDG, skin-specific DC, Langerhans; row 2 —pDC, monocyte, monocyte/myeloid, NK cell, T cell; row 3—B cell, GC B cell, plasma cell, platelet, erythrocyte; row 4: endothelial cell, fibroblast, keratinocyte, melanocyte. In FIG. 32B the panels show GSVA scores for pathways, in each row from left to right: row 1 (top row)—IFN, IL-1 cytokines, IL-12 complex, T cell IL-12 signature, IL-12, IL-17 complex; row 2-IL-21 complex, IL-23 complex, T cell IL-23 signature, TGFB fibroblast, TNF, Th17; row 3—anti-inflammation, complement proteins, inflammasome, ROS production, apoptosis, cell cycle; row 4-immunoproteasome, proteasome, unfolded protein, glycolysis, pentose phosphate, TCA cycle; row 5—OXPHOS, FAAO, FABO, AA metabolism, peroxisome.

FIGS. 33A-33B show that analysis of cellular and molecular pathway signatures in lesional AD shows increased expression of skin-specific dendritic cell, B cell and IL12 inflammatory pathway gene signatures. GSVA enrichment scores of (FIG. 33A) cellular gene signatures and (FIG. 33B) pathway gene signatures in two datasets including AD samples and control samples. The number of AD samples per dataset that lie −1 standard deviation of the average of the control samples is denoted on the first subtext line. The number of AD samples per dataset that lie+1 standard deviation of the average of the control samples is denoted on the second subtext line. Welch's T-test: *p<0.05; **p<0.01; ***p<0.001; ****p<0.0001, as indicated where observed for a given pair of violin plots by a bracket and corresponding number of asterisks above the plots. Plots for AD samples are the right plot of each pair of violin plots. Plots for control samples (CTL) are the left plot of each pair of violin plots. In each panel, each pair of violin plots corresponds to analysis of NCBI Gene Expression Omnibus dataset, from left to right, GSE130588 and GSE121212. Dotted horizontal line indicates GSVA enrichment score of 0, with positive scores above and negative scores below. In FIG. 33A the panels show GSVA scores for cell types, in each row from left to right: row 1 (top row)—granulocyte, neutrophil, LDG, skin-specific DC, Langerhans; row 2 —pDC, monocyte, monocyte/myeloid, NK cell, T cell; row 3—B cell, GC B cell, plasma cell, platelet, erythrocyte; row 4: endothelial cell, fibroblast, keratinocyte, melanocyte. In FIG. 33B the panels show GSVA scores for pathways, in each row from left to right: row 1 (top row)—IFN, IL-1 cytokines, IL-12 complex, T cell IL-12 signature, IL-12, IL-17 complex; row 2-IL-21 complex, IL-23 complex, T cell IL-23 signature, TGFB fibroblast, TNF, Th17; row 3—anti-inflammation, complement proteins, inflammasome, ROS production, apoptosis, cell cycle; row 4-immunoproteasome, proteasome, unfolded protein, glycolysis, pentose phosphate, TCA cycle; row 5—OXPHOS, FAAO, FABO, AA metabolism, peroxisome.

FIGS. 34A-34B show that analysis of cellular and molecular pathway signatures in lesional SSc samples show increased expression of myeloid-specific cell and TGFβ fibroblast gene signatures. GSVA enrichment scores of (FIG. 34A) cellular gene signatures and (FIG. 34B) pathway gene signatures in three datasets including SSc samples and control samples. The number of SSc samples per dataset that lie −1 standard deviation of the average of the control samples is denoted on the first subtext line. The number of SSc samples per dataset that lie +1 standard deviation of the average of the control samples is denoted on the second subtext line. Welch's T-test: *p<0.05; **p<0.01; ***p<0.001; ****p<0.0001, as indicated where observed for a given pair of violin plots by a bracket and corresponding number of asterisks above the plots. Plots for SSc samples are shown in dark gray (the right plot of each pair of violin plots). Plots for control samples (CTL) are shown in light gray (the left plot of each pair of violin plots). In each panel, each pair of violin plots corresponds to analysis of NCBI Gene Expression Omnibus dataset, from left to right, GSE58095, GSE95065, and GSE130955. Dotted horizontal line indicates GSVA enrichment score of 0, with positive scores above and negative scores below. In FIG. 34A the panels show GSVA scores for cell types, in each row from left to right: row 1 (top row)—granulocyte, neutrophil, LDG, skin-specific DC, Langerhans; row 2 —pDC, monocyte, monocyte/myeloid, NK cell, T cell; row 3—B cell, GC B cell, plasma cell, platelet, erythrocyte; row 4: endothelial cell, fibroblast, keratinocyte, melanocyte. In FIG. 34B the panels show GSVA scores for pathways, in each row from left to right: row 1 (top row)—IFN, IL-1 cytokines, IL-12 complex, T cell IL-12 signature, IL-12, IL-17 complex; row 2—IL-21 complex, IL-23 complex, T cell IL-23 signature, TGFB fibroblast, TNF, Th17; row 3—anti-inflammation, complement proteins, inflammasome, ROS production, apoptosis, cell cycle; row 4—immunoproteasome, proteasome, unfolded protein, glycolysis, pentose phosphate, TCA cycle; row 5—OXPHOS, FAAO, FABO, AA metabolism, peroxisome.

FIGS. 35A-35H shows ML effectively classifies lesional skin samples from DLE, PSO, AD, and SSc. ROC curve (FIG. 35A) and PR curve (FIG. 35B) of lesional DLE, lesional PSO, lesional AD, and lesional SSc samples compared to pooled control samples using all cellular and pathway gene signatures. Top 15 features important in classifying: (FIG. 35C) lesional DLE, (FIG. 35D) lesional PSO (FIG. 35E), lesional AD, and (FIG. 35F) lesional SSc from pooled control samples using Gini feature importance. (FIG. 35G) Comparison of the top 15 features for classifying each lesional disease compared to control using Gini feature importance. (FIG. 35H, Table 7) Classification metrics to properly separate DLE, PSO, AD or SSc and control samples using all 48 (top) or the top 15 (bottom) cellular and pathway gene signatures. Refer to Tables 5A-B for ML details. Collinear features were removed (FIG. 37). The AUC values of the ROC curves (FIG. 35A) for lesional DLE vs. control, lesional PSO vs. control, lesional AD vs. control, and lesional SSc vs. control classification are 0.977, 0.977, 0.963 and 0.965 respectively. The AUC values of the PR curves (FIG. 35B) for lesional DLE vs. control, lesional PSO vs. control, lesional AD vs. control, and lesional SSc vs. control are 0.972, 0.982, 0.970 and 0.968 respectively. Top 15 features important in classifying lesional DLE vs. control (FIG. 35C) are (in order of gini index, highest to lowest) IFN, TNF, IL-23 Complex, Plasma Cell, T Cell IL-12 signature, IL-12 Complex, Monocyte, Inflammasome, Unfolded Protein, B Cell, T Cell, pDC, Anti-inflammation, Immunoproteasome, and T Cell IL-23 signature. Top 15 features important in classifying lesional PSO vs. control (FIG. 35D) are (in order of gini index, highest to lowest) Cell Cycle, TNF, IL-12 Complex, Inflammasome, IFN, IL-23 complex, Apoptosis, Keratinocyte, Anti-inflammation, T Cell IL-23 signature, Proteasome, Unfolded Protein, Neutrophil, Pentose Phosphate, and Plasma Cell. Top 15 features important in classifying lesional AD vs. control (FIG. 35E) are (in order of gini index, highest to lowest) IL-12 Complex, TNF, IFN, T Cell IL-12 signature, Anti-inflammation, Inflammasome, Plasma Cell, IL-23 Complex, IL-21 Complex, T Cell IL-23 signature, Glycolysis, Immunoproteasome, Monocyte/Myeloid Cell, Cell Cycle and Apoptosis. Top 15 features important in classifying lesional SSc vs. control (FIG. 35F) are (in order of gini index, highest to lowest) Plasma Cell, IFN, TNF, ROS production, Unfolded Protein, IL-12 Complex, Anti-inflammation, Apoptosis, TGFB Fibroblast, IL-23 Complex, Skin-specific DC, Granulocyte, pDC, IL-17 Complex, and T Cell IL-23 Signature. From FIG. 35G shared features between Lesional DLE, PSO, AD and SSc are IFN, TNF, IL-23 Complex, Plasma Cell, IL-12 Complex, Anti-inflammation, and T Cell IL-23 Signature; Lesional DLE only features are Monocyte, B cell and T cell; Lesional AD only features are IL-21 Complex, Glycolysis, Monocyte/Myeloid Cell; Lesional PSO only features are Keratinocyte, Proteasome, Neutrophil, and Pentose Phosphate; and Lesional SSc only features are ROS production, TGFB fibroblast, skin-specific DC, Granulocyte and IL-17 Complex.

FIGS. 36A-36E shows ML accurately classifies lesional skin and control skin samples. ROC curve and PR curve of all ML algorithms to separate lesional samples from healthy control samples using all cellular and pathway gene signatures/features. ML classifiers include: logistic regression (LR, blue), K-nearest neighbors (KNN, orange), random forest (RF, green), naïve bayes (NB, red), support-vector machine (SVM, purple) and gradient boosting (GB, brown). (FIG. 36A) DLE versus control; (FIG. 36B) PSO versus control; (FIG. 36C) AD versus control; and (FIG. 36D) SSc versus control. FIG. 36E: Classification metrics including sensitivity, specificity, Cohen Kappa score, precision, f-1 score and accuracy to properly separate lesional disease samples (DLE, PSO, AD or SSc) from healthy control samples with each ML classifier (Table 8). Refer to Tables 5A-B for details about ML. Collinear features were removed (FIG. 37). The AUC values of the ROC curves (FIG. 36A) for lesional DLE vs. control classification, for ML classifiers LR, KNN, RF, NB, SVM, and GB are 0.959, 0.975, 0.977, 0.974, 0.959, and 0.949 respectively. The AUC values of the PR curves (FIG. 36A) for lesional DLE vs. control classification, for ML classifiers LR, KNN, RF, NB, SVM, and GB are 0.954, 0.963, 0.972, 0.971, 0.962, and 0.944 respectively. The AUC values of the ROC curves (FIG. 36B) for lesional PSO vs. control classification, for ML classifiers LR, KNN, RF, NB, SVM, and GB are 0.986, 0.972, 0.977, 0.983, 0.984, and 0.978 respectively. The AUC values of the PR curves (FIG. 36B) for lesional PSO vs. control classification, for ML classifiers LR, KNN, RF, NB, SVM, and GB are 0.988, 0.980, 0.982, 0.986, 0.986, and 0.982 respectively. The AUC values of the ROC curves (FIG. 36C) for lesional AD vs. control classification, for ML classifiers LR, KNN, RF, NB, SVM, and GB are 0.955, 0.936, 0.963, 0.945, 0.945, and 0.968 respectively. The AUC values of the PR curves (FIG. 36C) for lesional AD vs. control classification, for ML classifiers LR, KNN, RF, NB, SVM, and GB are 0.962, 0.961, 0.970, 0.959, 0.966, and 0.973 respectively. The AUC values of the ROC curves (FIG. 36D) for lesional SSc vs. control classification, for ML classifiers LR, KNN, RF, NB, SVM, and GB are 0.964, 0.967, 0.965, 0.952, 0.980, and 0.946 respectively. The AUC values of the PR curves (FIG. 36D) for lesional SSc vs. control classification, for ML classifiers LR, KNN, RF, NB, SVM, and GB are 0.967, 0.972, 0.968, 0.956, 0.983, and 0.955 respectively.

FIGS. 37A-37D show correlated features from cellular and pathway signatures used to extract collinear features for lesional ML binary classifications. Correlation plots of GSVA enrichment scores of pooled control samples and pooled lesional (FIG. 37A) DLE, (FIG. 37B) PSO, (FIG. 37C) AD and (FIG. 37D) SSc samples. Black boxes indicate collinear samples with Pearson correlation coefficient greater than 0.8, then the feature with the lower correlation was removed using a greedy elimination approach.

FIGS. 38A-38B show that direct comparison of DLE and PSO samples using GSVA shows key differences in enrichment of inflammatory cell and pathway signatures. (FIG. 38A) Hierarchical clustering (k=4 clusters) of GSVA enrichments scores of cellular and pathway gene signatures in two datasets including DLE, PSO and healthy control samples. (FIG. 38B) Heatmap of GSVA enrichment scores of DLE compared to PSO samples in two datasets of cellular (left) and pathway (right) gene signatures. Heatmap visualization uses red (enriched signature, >0) and blue (decreased signature, <0). Welch's t-test: *p<0.05; **p<0.01; ***p<0.001; ****p<0.0001.

FIGS. 39A-39F show that ML classification of DLE versus PSO, AD, and SSc confirms distinct disease-specific gene signatures. (FIG. 39A) ROC curve and (FIG. 39B) PR curve of lesional DLE samples compared to lesional PSO (purple) samples and lesional DLE samples compared to lesional AD samples (orange) using all cellular and pathway gene signatures. Top 15 features important in classifying (FIG. 39C) lesional DLE and lesional PSO, (FIG. 39D) lesional DLE and lesional AD, and FIG. 39E) lesional DLE and lesional SSc using Gini feature importance. (FIG. 39F, Table 9) Classification metrics to properly separate lesional DLE samples and lesional PSO or lesional AD samples using all 48 (top) or the top 15 (bottom) cellular and pathway gene signatures. Refer to Table 5A-B for ML details. Collinear features were removed (FIG. 41). The AUC values of the ROC curves (FIG. 39A) for lesional DLE vs. PSO, lesional DLE vs. AD, and lesional DLE vs. SSc classification are 0.902, 0.816, and 0.774 respectively. The AUC values of the PR curves (FIG. 39B) for lesional DLE vs. PSO, lesional DLE vs. AD, and lesional DLE vs. SSc classification are 0.845, 0.754, and 0.776 respectively. Top 15 features important in classifying lesional DLE vs. PSO (FIG. 39C) are (in order of gini index, highest to lowest) Amino Acid Metabolism, Fibroblast, Keratinocyte, NK Cell, Granulocyte, Cell Cycle, Proteasome, Plasma Cell, pDC, Pentose Phosphate, IL-12, Monocyte, OXPHOS, Fatty Acid Alpha Oxidation and Glycolysis. Top 15 features important in classifying lesional DLE vs. AD (FIG. 39D) are (in order of gini index, highest to lowest) Glycolysis, TGFB Fibroblast, Langerhans Cell, Low Density Granulocyte, Cell Cycle, Melanocyte, Fibroblast, Complement Proteins, Amino Acid Metabolism, pDC, IFN, Monocyte, IL-21 Complex, Platelet and IL-12 complex. Top 15 features important in classifying lesional DLE vs. SSc (FIG. 39E) are (in order of gini index, highest to lowest) Th17, TGFB Fibroblast, IL-12, IFN, Fibroblast, T Cell, Low Density Granulocyte, Proteasome, Inflammasome, Glycolysis, ROS production, T Cell IL-23 Signature, pDC, IL-21 Complex, and Langerhans Cell.

FIGS. 40A-40D show that ML accurately classifies lesional DLE from lesional PSO, AD and SSc. ROC curve and PR curve of all ML algorithms to separate lesional DLE from other inflammatory skin diseases using all cellular and pathway gene signatures/features. ML classifiers include: logistic regression (LR, blue), K-nearest neighbors (KNN, orange), random forest (RF, green), naïve bayes (NB, red), support-vector machine (SVM, purple) and gradient boosting (GB, brown). (FIG. 40A) DLE versus PSO; (FIG. 40B) DLE versus AD; and (FIG. 40C) DLE versus SSc. (FIG. 40D, Table 10) Classification metrics including sensitivity, specificity, Cohen Kappa score, precision, f-1 score and accuracy to properly separate lesional DLE samples from lesional PSO, AD, and SSc samples with each ML classifier. Refer to Table 5A-B for details about ML. Collinear features were removed (FIG. 41). The AUC values of the ROC curves (FIG. 40A) for lesional DLE vs. PSO classification, for ML classifiers LR, KNN, RF, NB, SVM, and GB are 0.909, 0.919, 0.902, 0.853, 0.936, and 0.890 respectively. The AUC values of the PR curves (FIG. 40A) for lesional DLE vs. PSO classification, for ML classifiers LR, KNN, RF, NB, SVM, and GB are 0.851, 0.901, 0.845, 0.805, 0.907, and 0.849 respectively. The AUC values of the ROC curves (FIG. 40B) for lesional DLE vs. AD classification, for ML classifiers LR, KNN, RF, NB, SVM, and GB are 0.715, 0.911, 0.816, 0.780, 0.879, and 0.837 respectively. The AUC values of the PR curves (FIG. 40B) for lesional DLE vs. AD classification, for ML classifiers LR, KNN, RF, NB, SVM, and GB are 0.693, 0.880, 0.754, 0.755, 0.864, and 0.793 respectively. The AUC values of the ROC curves (FIG. 40C) for lesional DLE vs. SSc classification, for ML classifiers LR, KNN, RF, NB, SVM, and GB are 0.720, 0.816, 0.774, 0.689, 0.805, and 0.784 respectively. The AUC values of the PR curves (FIG. 40C) for lesional DLE vs. SSc classification, for ML classifiers LR, KNN, RF, NB, SVM, and GB are 0.790, 0.838, 0.776, 0.745, 0.846, and 0.802 respectively.

FIGS. 41A-41C show correlated features from cellular and pathway signatures used to extract collinear features for lesional ML binary classifications compared to DLE. Correlation plots of GSVA enrichment scores of lesional DLE and lesional (FIG. 41A) PSO, (FIG. 41B) AD and (FIG. 41C) SSc samples. Correlations outlined in black were reduced to only include one feature. Black boxes indicate collinear samples with Pearson correlation coefficient greater than 0.8, then the feature with the lower correlation was removed using a greedy elimination approach.

FIGS. 42A-42B show GSVA enrichment of lesional skin compared to nonlesional skin. Hedges' g effect sizes of GSVA enrichment scores for paired lesional and nonlesional samples, including two DLE, four AD and three PSO datasets using (FIG. 42A) cellular gene signatures and (FIG. 42B) pathway gene signatures. Lesional samples were compared to their respective nonlesional paired samples in DLE, AD and PSO. Heatmap visualization uses red (enriched signature, >0) and blue (decreased signature, <0). Paired t-test: *p<0.05; **p<0.01; ***p<0.001; ****p<0.0001.

FIGS. 43A-43G show that ML classification reveals nonlesional skin of DLE, PSO, and AD is distinct from control skin. (FIG. 43A) ROC curve and (FIG. 43B) PR curve of nonlesional DLE, nonlesional PSO, and nonlesional AD samples compared to pooled control samples using all cellular and pathway gene signatures. The top 15 features important in classifying (FIG. 43C) nonlesional DLE, (FIG. 43D) nonlesional PSO and (FIG. 43E) nonlesional AD and control samples using Gini feature importance. (FIG. 43F) Comparison of the top 15 features for classifying each nonlesional disease compared to control using Gini feature importance. (FIG. 43G, Table 11) Classification metrics to properly separate nonlesional DLE and control samples, nonlesional PSO and control samples, as well as nonlesional AD and control samples using all 48 (top) or the top 15 (bottom) cellular and pathway gene signatures. Refer to Tables 5A-B for ML details. Collinear features were removed (FIG. 46). The AUC values of the ROC curves (FIG. 43A) for non-lesional DLE vs. control, non-lesional PSO vs. control, and non-lesional AD vs. control, classification are 0.996, 0.859, and 0.922 respectively. The AUC values of the PR curves (FIG. 43B) for non-lesional DLE vs. control, non-lesional PSO vs. control, and non-lesional AD vs. control, are 0.997, 0.902, and 0.941 respectively. Top 15 features important in classifying non-lesional DLE vs. control (FIG. 43C) are (in order of gini index, highest to lowest) Unfolded Protein, Langerhans Cell, NK Cell, Plasma Cell, IL-12, B Cell, Fatty Acid Beta Oxidation, Melanocyte, IL-12 Complex, Inflammasome, Apoptosis, Peroxisome, IL-21 Complex, Amino Acid Metabolism, and TNF. Top 15 features important in classifying non-lesional PSO vs. control (FIG. 43D) are (in order of gini index, highest to lowest) Amino Acid Metabolism, Cell Cycle, IL-17 Complex, NK Cell, Th17, OXPHOS, Proteasome, TGFB Fibroblast, Low Density Granulocyte, pDC, Skin-specific DC, Neutrophil, Unfolded Protein, Apoptosis, and GC B Cell. Top 15 features important in classifying non-lesional AD vs. control (FIG. 43E) are (in order of gini index, highest to lowest) OXPHOS, Anti-inflammation, Granulocyte, Keratinocyte, Apoptosis, Proteasome, Low-density Granulocyte, Pentose Phosphate, Monocyte/Myeloid Cell, Plasma Cell, Neutrophill, T Cell IL-23 Signature, IL-1 Cytokines, Erythrocyte and Melanocyte. From FIG. 43F shared features between non-lesional DLE, PSO, and AD is Apoptosis; non-lesional DLE only features are Langerhans Cell, IL-12, B Cell, Fatty Acid Beta Oxidation, IL-12 Complex, Inflammasome, Peroxisome, IL-21 Complex, and TNF; non-lesional AD only features are Anti-inflammation, Granulocyte, Keratinocyte, Pentose Phosphate, Monocyte/Myeloid Cell, T Cell IL-23 Signature, IL-1 Cytokines, and Erythrocyte; and non-lesional PSO only features are Cell Cycle, IL-17 Complex, Th17, TGFB Fibroblast, pDC, Skin-specific DC, and GC B Cell.

FIG. 44A-44D show that ML accurately separates nonlesional skin and control skin groups. ROC curve and PR curve of all machine learning classification algorithms to separate nonlesional samples from healthy control samples using all cellular and pathway gene signatures/features. ML classifiers include: logistic regression (LR, blue), K-nearest neighbors (KNN, orange), random forest (RF, green), naïve bayes (NB, red), support-vector machine (SVM, purple) and gradient boosting (GB, brown). (FIG. 44A) DLE versus control; (FIG. 44B) PSO versus control; and (FIG. 44C) AD versus control. (FIG. 44D, Table 12) Classification metrics including sensitivity, specificity, Cohen's Kappa score, precision, f-1 score and accuracy to properly separate nonlesional disease samples (DLE, PSO or AD) from healthy control samples with each ML classifier. Refer to Tables 5A-B for details about ML. Collinear features were removed (FIG. 46). The AUC values of the ROC curves (FIG. 44A) for non-lesional DLE vs. control classification, for ML classifiers LR, KNN, RF, NB, SVM, and GB are 0.934, 0.958, 0.996, 0.942, 0.994, and 0.983 respectively. The AUC values of the PR curves (FIG. 44A) for non-lesional DLE vs. control classification, for ML classifiers LR, KNN, RF, NB, SVM, and GB are 0.912, 0.963, 0.997, 0.968, 0.995, and 0.987 respectively. The AUC values of the ROC curves (FIG. 44B) for non-lesional PSO vs. control classification, for ML classifiers LR, KNN, RF, NB, SVM, and GB are 0.840, 0.889, 0.859, 0.822, 0.883, and 0.832 respectively. The AUC values of the PR curves (FIG. 44B) for non-lesional PSO vs. control classification, for ML classifiers LR, KNN, RF, NB, SVM, and GB are 0.885, 0.930, 0.902, 0.856, 0.925, and 0.886 respectively. The AUC values of the ROC curves (FIG. 44C) for non-lesional AD vs. control classification, for ML classifiers LR, KNN, RF, NB, SVM, and GB are 0.813, 0.836, 0.922, 0.771, 0.940, and 0.894 respectively. The AUC values of the PR curves (FIG. 44C) for non-lesional AD vs. control classification, for ML classifiers LR, KNN, RF, NB, SVM, and GB are 0.805, 0.842, 0.941, 0.793, 0.931, and 0.904 respectively.

FIGS. 45A-45E show nonlesional DLE is distinct from PSO and AD. (FIG. 45A) ROC curve and (FIG. 45B) PR curve of nonlesional DLE samples compared to nonlesional PSO (purple) samples and nonlesional DLE samples compared to nonlesional AD samples (orange) using all cellular and pathway gene signatures. Top 15 features important in classifying (FIG. 45C) nonlesional DLE and nonlesional PSO and (FIG. 45D) nonlesional DLE and nonlesional AD using Gini feature importance. (FIG. 45E, Table 13) Classification metrics to properly separate DLE samples and PSO or AD samples using all 48 (top) or the top 15 (bottom) cellular and pathway gene signatures. Refer to Tables 5A-B for ML details. Collinear features were removed (FIG. 50). The AUC values of the ROC curves (FIG. 45A) for non-lesional DLE vs. PSO, and non-lesional DLE vs. AD, classification are 1 and 0.990 respectively. The AUC values of the PR curves (FIG. 45B) for non-lesional DLE vs. PSO, and non-lesional DLE vs. AD classification are 1 and 0.989 respectively. Top 15 features important in classifying non-lesional DLE vs. PSO (FIG. 45C) are (in order of gini index, highest to lowest) NK Cell, Amino Acid Metabolism, Plasma Cell, pDC, Inflammasome, Monocyte/Myeloid Cell, Langerhans Cell, B Cell, TNF, Unfolded Protein, TCA Cycle, T Cell IL-12 Signature, Keratinocyte, IL-12 Complex, and Melanocyte. Top 15 features important in classifying non-lesional DLE vs. AD (FIG. 45D) are (in order of gini index, highest to lowest) Inflammasome, NK Cell, Unfolded Protein, B Cell, pDC, IL-12 Complex, TNF, Langerhans Cell, Plasma Cell, Anti-inflammation, Amino Acid Metabolism, Melanocyte, Monocyte/Myeloid Cell, IL-21 Complex, and Immunoproteasome.

FIGS. 46A-46C show correlated features from cellular and pathway signatures used to extract collinear features for nonlesional ML binary classification. Correlation plots of GSVA enrichment scores of control samples and nonlesional (FIG. 46A) DLE, (FIG. 46B) PSO and (FIG. 46C) AD samples. Correlations outlined in black were reduced to only include one feature. Black boxes indicate collinear samples with Pearson correlation coefficient greater than 0.8, then the feature with the lower correlation was removed using a greedy elimination approach.

FIG. 47A-47C show ML distinguishes nonlesional DLE from nonlesional PSO and nonlesional AD. ROC curve and PR curve of all machine learning classification algorithms to separate nonlesional DLE from other inflammatory skin diseases using all cellular and pathway gene signatures/features. ML classifiers include: logistic regression (LR, blue), K-nearest neighbors (KNN, orange), random forest (RF, green), naïve bayes (NB, red), support-vector machine (SVM, purple) and gradient boosting(GB, brown). (FIG. 47A) DLE versus PSO and (FIG. 47B) DLE versus AD. (FIG. 47C, Table 14) Classification metrics including sensitivity, specificity, Cohen Kappa score, precision, f-1 score and accuracy to properly separate nonlesional DLE samples from nonlesional PSO and nonlesional AD samples with each ML classifier. Refer to Tables 5A-B for details about ML. Collinear features were removed (FIG. 50). The AUC values of the ROC curves (FIG. 47A) for non-lesional DLE vs. PSO classification, for ML classifiers LR, KNN, RF, NB, SVM, and GB are 0.974, 0.953, 1, 0.982, 1, and 0.971 respectively. The AUC values of the PR curves (FIG. 47A) for non-lesional DLE vs. PSO classification, for ML classifiers LR, KNN, RF, NB, SVM, and GB are 0.944, 0.947, 1, 0.986, 1, and 0.963 respectively. The AUC values of the ROC curves (FIG. 47B) for non-lesional DLE vs. AD classification, for ML classifiers LR, KNN, RF, NB, SVM, and GB are 0.983, 0.953, 0.990, 0.961, 0.997, and 0.974 respectively. The AUC values of the PR curves (FIG. 47B) for non-lesional DLE vs. AD classification, for ML classifiers LR, KNN, RF, NB, SVM, and GB are 0.983, 0.946, 0.989, 0.969, 0.997, and 0.975 respectively.

FIG. 48A-48D show ML classification of nonlesional PSO and AD. (FIG. 48A) ROC curve and PR curve of all ML classification algorithms to separate nonlesional PSO from nonlesional AD samples using all cellular and pathway gene signatures/features. ML classifiers include: logistic regression (LR, blue), K-nearest neighbors (KNN, orange), random forest (RF, green), naïve bayes (NB, red), support-vector machine (SVM, purple) and gradient boosting (GB, brown). (FIG. 48B) Top 15 features important in classifying nonlesional PSO from nonlesional AD using Gini feature importance. (FIG. 48C, Table 15) Classification metrics including sensitivity, specificity, Cohen Kappa score, precision, f-1 score and accuracy to properly separate nonlesional PSO samples from nonlesional AD samples with each ML classifier. (FIG. 48D) Correlation plots of GSVA enrichment scores of nonlesional PSO and nonlesional AD samples. Black boxes indicate collinear samples with Pearson correlation coefficient greater than 0.8, then the feature with the lower correlation was removed using a greedy elimination approach. The AUC values of the ROC curves (FIG. 48A) for non-lesional AD vs. PSO classification, for ML classifiers LR, KNN, RF, NB, SVM, and GB are 0.684, 0.830, 0.801, 0.807, 0.739, and 0.824 respectively. The AUC values of the PR curves (FIG. 48A) for non-lesional AD vs. PSO classification, for ML classifiers LR, KNN, RF, NB, SVM, and GB are 0.682, 0.867, 0.841, 0.854, 0.767, and 0.851 respectively. Top 15 features important in classifying non-lesional AD vs. PSO (FIG. 48D) are (in order of gini index, highest to lowest) Amino Acid Metabolism, IL-23 Complex, Cell Cycle, Glycolysis, OXPHOS, Low-density Granulocyte, IL-17 Complex, Fibroblast, IL-12 Complex, NK Cell, Proteasome, T Cell IL-12 Signature, Inflammasome, IL-21 Complex, and Monocyte.

FIGS. 49A-49B show nonlesional skin is characterized by upregulation of unique cellular and pathway signatures. (FIG. 49A) Hedges' g effect sizes of cellular (left) and pathway (right) gene signatures for pooled nonlesional disease samples compared to pooled control samples DLE, PSO and AD datasets. Heatmap visualization uses red (enriched signature, >0) and blue (decreased signature, <0). Welch's t-test: *p<0.05; **p<0.01; ***p<0.001; ****p<0.0001. (FIG. 49B) Comparison of the most important features determined by ML that are also statistically significant by Z-score GSVA of nonlesional skin versus controls for nonlesional DLE (left), nonlesional PSO (middle) and nonlesional AD (right). 40 features were used in the nonlesional Z-score GSVA, only these features were used in the comparison to nonlesional ML.

FIGS. 50A-50B show correlated features from cellular and pathway signatures used to extract collinear features for nonlesional ML binary classification compared to DLE. Correlation plots of GSVA enrichment scores of nonlesional DLE and (FIG. 50A) nonlesional PSO and (FIG. 50B) nonlesional AD samples. Black boxes indicate collinear samples with Pearson correlation coefficient greater than 0.8, then the feature with the lower correlation was removed using a greedy elimination approach.

FIGS. 51A-51B show that analysis of cellular and molecular pathway signatures in nonlesional DLE (NL DLE) shows upregulation of B cell, plasma cell and fatty acid metabolism gene signatures. GSVA enrichment scores using Z-scores of (FIG. 51A) cellular gene signatures and (FIG. 51B) pathway gene signatures in nonlesional DLE and control samples. The number of nonlesional DLE samples per dataset that lie −1 standard deviation of the average of the control samples is denoted on the first subtext line. The number of DLE samples per dataset that lie+1 standard deviation of the average of the control samples is denoted on the second subtext line. Welch's T-test: *p<0.05; **p<0.01; ***p<0.001; ****p<0.0001, as indicated where observed for a given pair of violin plots by a bracket and corresponding number of asterisks above the plots. Plots for NL DLE samples are shown in dark gray (the right plot of each pair of violin plots). Plots for control samples (CTL) are shown in light gray (the left plot of each pair of violin plots). Dotted horizontal line indicates GSVA enrichment score of 0, with positive scores above and negative scores below. In FIG. 51A the panels show GSVA scores for cell types, in each row from left to right: row 1 (top row)—granulocyte, neutrophil, LDG, skin-specific DC, Langerhans, monocyte, monocyte/myeloid, NK cell, T cell, B cell, GC B cell; row 2—plasma cell, platelet, endothelial cell, fibroblast, keratinocyte, melanocyte. In FIG. 51B the panels show GSVA scores for pathways, in each row from left to right: row 1 (top row)—IFN, T cell IL-12 signature, IL-12, IL-17 complex; T cell IL-23 signature, TGFB fibroblast, TNF, Th17, anti-inflammation, complement proteins; row 2-inflammasome, ROS production, apoptosis, cell cycle, proteasome, unfolded protein, glycolysis, pentose phosphate, TCA cycle, OXPHOS; row 3—FAAO, FABO, AA metabolism, peroxisome.

FIGS. 52A-52B show that analysis of cellular and molecular pathway signatures in nonlesional PSO (NL PSO) shows upregulation of innate immune cell and IL-17 gene signatures. GSVA enrichment scores using Z-scores of (FIG. 52A) cellular gene signatures and (FIG. 52B) pathway gene signatures in nonlesional PSO and control samples. The number of nonlesional PSO samples per dataset that lie −1 standard deviation of the average of the control samples is denoted on the first subtext line. The number of PSO samples per dataset that lie+1 standard deviation of the average of the control samples is denoted on the second subtext line. Welch's T-test: *p<0.05; ** p<0.01; ***p<0.001; ****p<0.0001, as indicated where observed for a given pair of violin plots by a bracket and corresponding number of asterisks above the plots. Plots for NL PSO samples are shown as the right plot of each pair of violin plots. Plots for control samples (CTL) are shown as the left plot of each pair of violin plots. Dotted horizontal line indicates GSVA enrichment score of 0, with positive scores above and negative scores below. In FIG. 52A the panels show GSVA scores for cell types, in each row from left to right: row 1 (top row)—granulocyte, neutrophil, LDG, skin-specific DC, Langerhans, monocyte, monocyte/myeloid, NK cell, T cell, B cell, GC B cell; row 2-plasma cell, platelet, endothelial cell, fibroblast, keratinocyte, melanocyte. In FIG. 52B the panels show GSVA scores for pathways, in each row from left to right: row 1 (top row)—IFN, T cell IL-12 signature, IL-12, IL-17 complex; T cell IL-23 signature, TGFB fibroblast, TNF, Th17, anti-inflammation, complement proteins; row 2—inflammasome, ROS production, apoptosis, cell cycle, proteasome, unfolded protein, glycolysis, pentose phosphate, TCA cycle, OXPHOS; row 3—FAAO, FABO, AA metabolism, peroxisome.

FIGS. 53A-53B show that analysis of cellular and molecular pathway signatures in nonlesional AD (NL AD) shows upregulation of anti-inflammation, neutrophil, NK cell and Th17 gene signatures. GSVA enrichment scores using Z-scores of (FIG. 53A) cellular gene signatures and (FIG. 53B) pathway gene signatures in nonlesional AD and control samples. The number of nonlesional AD samples per dataset that lie −1 standard deviation of the average of the control samples is denoted on the first subtext line. The number of AD samples per dataset that lie+1 standard deviation of the average of the control samples is denoted on the second subtext line. Welch's T-test: *p<0.05; **p<0.01; ***p<0.001; ****p<0.0001, as indicated where observed for a given pair of violin plots by a bracket and corresponding number of asterisks above the plots. Plots for NL AD samples are shown as the right plot of each pair of violin plots. Plots for control samples (CTL) are shown as the left plot of each pair of violin plots. Dotted horizontal line indicates GSVA enrichment score of 0, with positive scores above and negative scores below. In FIG. 53A the panels show GSVA scores for cell types, in each row from left to right: row 1 (top row)—granulocyte, neutrophil, LDG, skin-specific DC, Langerhans, monocyte, monocyte/myeloid, NK cell, T cell, B cell, GC B cell; row 2—plasma cell, platelet, endothelial cell, fibroblast, keratinocyte, melanocyte. In FIG. 53B the panels show GSVA scores for pathways, in each row from left to right: row 1 (top row)—IFN, T cell IL-12 signature, IL-12, IL-17 complex; T cell IL-23 signature, TGFB fibroblast, TNF, Th17, anti-inflammation, complement proteins; row 2-inflammasome, ROS production, apoptosis, cell cycle, proteasome, unfolded protein, glycolysis, pentose phosphate, TCA cycle, OXPHOS; row 3—FAAO, FABO, AA metabolism, peroxisome.

FIGS. 54A-54B show analysis of cellular and molecular pathway signatures in nonlesional DLE using mean of Z-score. Box plots of the mean of Z-scores of genes for each sample and gene category for (FIG. 54A) cellular gene signatures and (FIG. 54B) pathway gene signatures in nonlesional DLE and control samples. Welch's T-test: *p<0.05; **p<0.01; ***p<0.001; **** p<0.0001, as indicated where observed for a given pair of box plots by a bracket and corresponding number of asterisks above the plots. Plots for NL DLE samples are shown as the right plot of each pair of box plots. Plots for control samples (CTL) are shown as the left plot of each pair of box plots. Dotted horizontal line indicates mean of Z-score of 0, with positive scores above and negative scores below. In FIG. 54A the panels show the mean of Z-scores for cell types, in each row from left to right: row 1 (top row)—granulocyte, neutrophil, skin-specific DC, Langerhans, monocyte, monocyte/myeloid, NK cell, T cell, B cell; row 2—plasma cell, platelet, endothelial cell, fibroblast, keratinocyte, melanocyte. In FIG. 54B the panels show mean of Z-scores for pathways, in each row from left to right: row 1 (top row)—IFN, T cell IL-12 signature, IL-12, IL-17 complex; T cell IL-23 signature, TGFB fibroblast, TNF, Th17, anti-inflammation, complement proteins; row 2-inflammasome, ROS production, apoptosis, cell cycle, proteasome, unfolded protein, glycolysis, pentose phosphate, TCA cycle, OXPHOS; row 3—FAAO, FABO, AA metabolism, peroxisome.

FIG. 55A-55B show analysis of cellular and molecular pathway signatures in nonlesional PSO using mean of Z-score. Box plots of the mean of Z-scores of genes for each sample and gene category for (FIG. 55A) cellular gene signatures and (FIG. 55B) pathway gene signatures in nonlesional PSO and control samples. Welch's T-test: *p<0.05; **p<0.01; ***p<0.001; **** p<0.0001, as indicated where observed for a given pair of box plots by a bracket and corresponding number of asterisks above the plots. Plots for NL PSO samples are shown as the right plot of each pair of box plots. Plots for control samples (CTL) are shown as the left plot of each pair of box plots. Dotted horizontal line indicates mean of Z-score of 0, with positive scores above and negative scores below. In FIG. 55A the panels show the mean of Z-scores for cell types, in each row from left to right: row 1 (top row)—granulocyte, neutrophil, skin-specific DC, Langerhans, monocyte, monocyte/myeloid, NK cell, T cell, B cell; row 2—plasma cell, platelet, endothelial cell, fibroblast, keratinocyte, melanocyte. In FIG. 55B the panels show mean of Z-scores for pathways, in each row from left to right: row 1 (top row)—IFN, T cell IL-12 signature, IL-12, IL-17 complex; T cell IL-23 signature, TGFB fibroblast, TNF, Th17, anti-inflammation, complement proteins; row 2-inflammasome, ROS production, apoptosis, cell cycle, proteasome, unfolded protein, glycolysis, pentose phosphate, TCA cycle, OXPHOS; row 3—FAAO, FABO, AA metabolism, peroxisome.

FIGS. 56A-56B show analysis of cellular and molecular pathway signatures in nonlesional AD using mean of Z-score. Box plots of the mean of Z-scores of genes for each sample and gene category for (FIG. 56A) cellular gene signatures and (FIG. 56B) pathway gene signatures in nonlesional AD (light yellow) and control samples (grey). Welch's T-test: *p<0.05; **p<0.01; ***p<0.001; ****p<0.0001, as indicated where observed for a given pair of box plots by a bracket and corresponding number of asterisks above the plots. Plots for NL AD samples are shown as the right plot of each pair of box plots. Plots for control samples (CTL) are shown as the left plot of each pair of box plots. Dotted horizontal line indicates a mean of Z-score of 0, with positive scores above and negative scores below. In FIG. 56A the panels show the mean of Z-scores for cell types, in each row from left to right: row 1 (top row)—granulocyte, neutrophil, skin-specific DC, Langerhans, monocyte, monocyte/myeloid, NK cell, T cell, B cell; row 2—plasma cell, platelet, endothelial cell, fibroblast, keratinocyte, melanocyte. In FIG. 56B the panels show mean of Z-scores for pathways, in each row from left to right: row 1 (top row)—IFN, T cell IL-12 signature, IL-12, IL-17 complex; T cell IL-23 signature, TGFB fibroblast, TNF, Th17, anti-inflammation, complement proteins; row 2—inflammasome, ROS production, apoptosis, cell cycle, proteasome, unfolded protein, glycolysis, pentose phosphate, TCA cycle, OXPHOS; row 3—FAAO, FABO, AA metabolism, peroxisome.

FIGS. 57A-57D show cellular and pathway enrichment in SCLE is quantitatively similar to enrichment observed in DLE. Hedges' g effect sizes of GSVA enrichment scores for (FIG. 57A) cellular gene signatures and (FIG. 57B) pathway gene signatures in lesional SCLE and control samples in three datasets. Hedges' g effect sizes of GSVA enrichment scores for (FIG. 57C) cellular gene signatures and (FIG. 57D) pathway gene signatures in lesional DLE and SCLE samples in three datasets. Heatmap visualization uses red (enriched signature, >0) and blue (decreased signature, <0). Welch's t-test: *p<0.05; **p<0.01; ***p<0.001; ****p<0.0001.

FIGS. 58A-58F show DLE and SCLE can be transcriptionally classified using ML. (FIG. 58A) Hierarchical clustering (k=4) of DLE and SCLE samples from three lupus datasets based on GSVA scores of cellular and pathway gene signatures in control. (FIG. 58B) Correlation plot of GSVA enrichment scores of lesional DLE and lesional SCLE samples. (FIG. 58C) ROC curve and (FIG. 58D) PR curve separating DLE and SCLE using ML classifiers, including: logistic regression (LR, blue), random forest (RF, orange), support-vector machine (SVM, green) and gradient boosting (GB, red). Random oversampling was used to adjust for class imbalance errors. (FIG. 58E) Top 15 features important in classifying DLE from SCLE using Ginifeature importance. (FIG. 58F, Table 16) Classification metrics including sensitivity, specificity, Cohen Kappa score, precision, f-1 score and accuracy to properly separate DLE and SCLE. Refer to Table 5A-B for details about ML. The AUC values of the ROC curves (FIG. 58C) for DLE vs. SCLE classification, for ML classifiers LR, RF, SVM, and GB are 0.828, 0.910, 0.924, and 0.901 respectively. The AUC values of the PR curves (FIG. 58D) for DLE vs. SCLE classification, for ML classifiers LR, RF, SVM, and GB are 0.838, 0.885, 0.914, and 0.874 respectively. Top 15 features important in classifying DLE vs. SCLE (FIG. 58E) are (in order of gini index, highest to lowest) Plasma Cell, Unfolded Protein, TNF, Apoptosis, T Cell IL-12 Signature, IL-23 Complex, Neutrophil, pDC, Complement Proteins, IL-1 Cytokines, Melanocyte, Monocyte/Myeloid Cell, Fatty Acid Beta Oxidation, Amino Acid Metabolism and GC B Cell.

FIG. 59 show stimulated keratinocyte signatures are highly enriched in skin inflammatory diseases. Hedges' g effect sizes of GSVA enrichment scores for disease samples compared to their respective healthy control samples in five DLE, three PSO, two AD and three SSc datasets using curated keratinocyte-curated cellular signatures treated with various types of cytokines and immune molecules. Heatmap visualization uses red (enriched signature, >0) and blue (decreased signature, <0). Welch's t-test: *p<0.05; **p<0.01; ***p<0.001; ****p<0.0001.

FIGS. 60A-60D show overabundance of correlated features from keratinocyte cell gene signatures. Correlation plot of GSVA enrichment scores to find keratinocyte gene signatures that are correlated to each other in (FIG. 60A) DLE and control samples; (FIG. 60B) PSO and control samples; (FIG. 60C) AD and control samples; and (FIG. 60D) SSc and control samples.

FIGS. 61A-61E show T cell subtype signatures are highly enriched in skin inflammatory diseases. GSVA enrichment scores for (FIG. 61A) T cell cellular signatures in disease samples compared to their respective healthy control samples in five DLE, three PSO, two AD and three SSc datasets. Heatmap visualization uses red (enriched signature, >0) and blue (decreased signature, <0). Welch's t-test: *p<0.05; **p<0.01; ***p<0.001; ****p<0.0001. Correlation plots of GSVA enrichment scores to find T cell gene signatures that are correlated to each other in (FIG. 61B) DLE and control samples; (FIG. 61C) PSO and control samples; (FIG. 61D) AD and control samples; and (FIG. 61E) SSc and control samples. In each of FIGS. 61B-61E: top to bottom and left to right: Dermal Aner/Act T cell, Dermal CD8 T cell, Dermal Tfh, Dermal Th1, Dermal Th17, Dermal Th2, Dermal Treg, label.

FIGS. 62A-62B show nonlesional skin from patients with inflammatory skin diseases manifests a specific set of pre-clinical, molecular abnormalities that predispose the development of both shared and unique clinical features in lesional DLE, PSO, AD and SSc after encountering an environmental trigger. FIG. 62A shows summary graphic detailing features determined by ML and upregulated in nonlesional skin or lesional skin of DLE, PSO, AD and SSc versus control as determined by GSVA. Some features are upregulated in both nonlesional and lesional skin. The bottom box shows important ML features upregulated by GSVA in lesional skin and shared among all four inflammatory skin diseases. Refer to Table 6 for details about comparison between GSVA and Z-score methods. FIG. 62B shows a summary of possible therapies of lesional skin diseases analyzed (left) and possible therapies for both lesional and nonlesional regions of each disease (right) based on molecular characterization. * delineates drugs in development.

FIGS. 63A-63E show ML classification of DLE versus PSO, AD, and SSc confirms distinct disease-specific gene signatures. Top 15 features important in classifying lesional DLE versus lesional PSO (FIG. 63A), lesional DLE versus lesional AD (FIG. 63B), lesional DLE versus lesional SSc (FIG. 63C), nonlesional DLE versus nonlesional PSO (FIG. 63D), and nonlesional DLE versus nonlesional AD (FIG. 63E) using SHAP values. Collinear features were removed.

FIG. 64 shows derivation of the inflammatory skin disease risk score to calculate activity of cellular and immune pathways in lesional skin diseases. Coefficients resulting from the logistic regression and ridge penalty model of 48 cellular and pathway coefficients run with 500 iterations.

FIGS. 65A-C show K-means clustering of CLE and SSc skin reveals molecular endotypes. K-means clustering of (FIG. 65A) DLE (GSE184989) and (FIG. 65B) SSc (GSE58095) using GSVA enrichment scores of cellular and pathway gene signatures. (FIG. 65C) Cosine similarity analysis to compare the molecular profiles of the endotypes derived from DLE to those of SSc. In FIGS. 65A and 65B, the modules listed from top to bottom (left vertical axis) are OXPHOS, TCA cycle, FABO, IL 12, TNF, Inflammasome, Proteasome, Unfolded protein, Apoptosis, pDC, T Cell IL 12 Signature, T Cell, Skin-specific DC, Keratinocyte, Plasma Cell, Endothelial Cell, Cell cycle, Peroxisome, Complement Proteins, Monocyte, Pentose Phosphate, TGFB Fibroblast, AA metabolism, Fibroblast, Glycolysis, Monocyte/Myeloid Cell, IL 17 Complex, IL 1 cytokines, Anti inflammation, IL 21 Complex, NK Cell, IFN, Immunoproteasome, ROS Production, Langerhans Cell, IL23 Complex, IL 12 Complex, FAAO, GC B Cell, Melanocyte, Granulocyte, Neutrophil, Th17, B Cell, LDG, Platelet, T Cell IL 23 Signature, and Erythrocyte.

FIGS. 66A-D show transcriptional analysis of immune populations in NZM2328 mice with acute GN (AGN). Individual sample gene expression from the glomeruli (FIGS. 66A&C) and tubulointerstitial tissue (FIGS. 66B&D) of CTL and AGN mice was analyzed by GSVA for enrichment of immune cells/inflammatory pathways (FIGS. 66A-B) and kidney tissue cells (FIGS. 66C-D). Enrichment scores are shown as violin plots. *p<0.05, **p<0.01, ***p<0.001.

FIGS. 67A-F show histologic and transcriptomic analysis of LN disease stages in the glomeruli of NZM2328 mice. FIGS. 67A-D show H&E staining of kidneys from normal/CTL (FIG. 67A) NZM2328 females and mice with acute (FIG. 67B), transitional (FIG. 67C), and chronic (FIG. 67D) stage GN. (FIG. 67E) Heatmap of GSVA scores for enrichment of immune cell and pathway gene signatures in the glomeruli of (control), AGN (acute stage glomerular nephritis), TGN (transitional glomerular nephritis), and CGN (chronic stage glomerular nephritis) mice. Asterisks (in black or white) indicate significant comparisons with CTL mice. (FIG. 67F) GSVA enrichment of podocytes gene signatures in cohorts shown in FIG. 67E. *p<0.05, **p<0.01, ***p<0.001, ****p<0.0001.

FIGS. 68A-D show immune profiling and kidney tissue analysis of LN disease stages in the TI of NZM2328 mice. (FIG. 68A) Heatmap of GSVA scores for enrichment of immune cell and pathway gene signatures in the TI of CTL, AGN, TGN, and CGN mice. Asterisks indicate significant comparisons with CTL mice. (FIG. 68B) GSVA enrichment of kidney tissue cell gene signatures in cohorts from FIG. 68A. (FIG. 68C) Log2 expression values of kidney tubule damage-associated genes for cohorts from FIG. 68A. (FIG. 68D) Linear regression between log 2 expression of kidney tubule damage genes and GSVA scores of kidney tubule cells. *p<0.05, **p<0.01, ***p<0.001.

FIGS. 69A-C show male NZM2328 mice lack inflammatory signature enrichment associated with progression to chronic GN. (FIG. 69A) Heatmap of GSVA scores for enrichment of immune cell and pathway gene signatures in the glomeruli of male CTL and AGN mice. Asterisks indicate significant comparisons with CTL mice. (FIG. 69B) GSVA enrichment of podocytes gene signatures in cohorts shown in FIG. 69A. (FIG. 69C) GSVA enrichment of signatures for estrogen-regulated and androgen-regulated genes in the glomeruli of female and male AGN mice. *p<0.05, **p<0.01.

FIGS. 70A-C show inflammatory gene signatures in the glomeruli of R27 mice differ from NZM2328 mice. (FIG. 70A) Bubbleplot depicting the overlap of DEGs up-regulated in the glomeruli of NZM2328 and R27 AGN mice with immunologic gene signatures. Bubble size indicates odds ratio and color indicates p-value of the comparison with CTL mice. Asterisks indicate statistically significant comparisons (shown in lower left of all cells except R27 pattern recognition receptor). (FIG. 70B) Heatmap of GSVA scores for enrichment of immune cell and pathway gene signatures in the glomeruli of R27 CTL and AGN mice. Asterisks (in black or white) indicate significant comparisons with CTL mice. (FIG. 70C) GSVA enrichment of podocytes gene signatures in cohorts shown in FIG. 70B. *p<0.05, **p<0.01, ***p<0.001.

FIGS. 71A-E show gene expression analysis of the TI of R27 mice indicates resistance to kidney tubule damage. (FIG. 71A) Bubbleplot depicting the overlap of DEGs up-regulated in the TI NZM2328 and R27 AGN mice with immunologic gene signatures. Bubble size indicates odds ratio and color indicates p-value of the comparison with CTL mice. Asterisks indicate statistically significant comparisons. (FIG. 71B) Heatmap of GSVA scores for enrichment of immune cell and pathway gene signatures in the TI of R27 CTL and AGN mice. Asterisks indicate significant comparisons with CTL mice. (FIG. 71C) GSVA enrichment of kidney tissue cell signatures in cohorts shown in FIG. 71B. (FIG. 71D) Log2 expression values of kidney tubule damage-associated genes for cohorts from FIG. 71B. (FIG. 71E) Linear regression between GSVA scores of kidney tubule cell and metabolic pathway gene signatures. *p<0.05, **p<0.01, ***p<0.001, ****p<0.0001.

FIGS. 72A-E show expression of chronic risk locus genes is associated with disease severity and kidney tubule resistance in NZM2328 and R27 AGN mice. (FIGS. 72A&B) Log2 expression values of immune receptor genes in the Cgnz1 risk locus from the glomeruli (FIG. 72A) and TI (FIG. 72B) of NZM2328 CTL, AGN, TGN, and CGN mice. (FIG. 72C) Linear regression between log 2 expression of Cgnz1 locus genes (x-axis) and GSVA scores (y-axis) of kidney tubule cells from the TI of R27 mice. All statistically significant correlations are shown. (FIG. 72D) Log2 expression values Cgnz1 locus genes from FIG. 72C in the TI of NZM2328 CTL, AGN, TGN, and CGN mice. (FIG. 72E) Linear regression between log 2 expression of Cgnz1 locus genes from FIG. 72C and GSVA scores of kidney tubule cells from the TI of NZM2328 mice. All statistically significant correlations are shown. *p<0.05, **p<0.01, ***p<0.001, ****p<0.0001.

FIG. 73. Schematic showing progression to CGN in NZM2328 AGN mice, and resistance to CGN in NZM2328.R27 AGN mice.

FIGS. 74A-B show clustering of GSVA enrichment scores in lupus kidneys of 76 patients with LN (BH11201) reveals four distinct endotypes of patients with LN. (FIG. 74A) Row and column hierarchical clustering of 76 patients with LN into four groups based upon gene expression of cellular and pathway gene modules. (FIG. 74B) Reordered clustering of LN patients in order of molecular disease severity from least to greatest. The columns represent individual patients that are grouped into four clusters (from left to right: black, coral, yellow, and purple). The rows represent gene modules indicative of immune/inflammatory cells, non-hematopoietic cells, and cellular metabolism. In FIGS. 74A-74B, the GSVA sets are (right vertical axis, top to bottom)—Amino Acid Metabolism, Fatty Acid Beta Oxidation, Kidney Proximal Convoluted Tubule, Fatty Acid Alpha Oxidation, Podocyte, TCA cycle, Kidney Cell, Kidney Distal Tubule, Oxidative Phosphorylation, Granulocyte, LDG, Platelet, NK Cell, Endothelial Cell, Kidney Loop of Henle Cell, Kidney Tubule Collecting Duct Cell, Pentose Phosphate, Glycolysis, pDC, Fibroblast, Mesangial Cell, Dendritic Cell, Anergic or Activated T Cell, GC B Cell, Plasma Cell, B Cell, Monocyte/Myeloid Cell, and T Cell.

FIGS. 75A-H show comparison of molecular endotypes with clinical features reveals some correlation between gene expression and histology. Distribution of (FIG. 75A) ISN/RPS (International Society of Nephrology/Renal Pathology Society; see, e.g., Markowitz and Agati, 2007, “The ISN/RPS 2003 classification of lupus nephritis: An assessment at 3 years,” Kidney International 71: 491-495, incorporated herein by reference in its entirety) classes in 46 patients with LN (three bars each for coral, yellow, purple and black, from left to right in each group: mesangial, proliferative, membranous), (FIG. 75B) positive or negative IgA deposition in 44 patients with LN (two bars each for coral, yellow, purple and black, from left to right in each group: negative, positive), (FIG. 75C) inactive or active SLEDAI in 32 patients with LN (two bars each for coral, yellow, purple and black, from left to right in each group: Inactive (SLEDAI<6), Active (SLEDAI≥6), (FIG. 75D) renal activity index in 49 patients with LN, and (FIG. 75E) renal chronicity index in 48 patients with LN among the LN endotypes. FIG. 75F shows proteinuria values (g/24 h) in 24 patients with LN. FIG. 75G shows the percent of 41 patients with LN having negative (“0”; left bar of each pair) or positive (“>0”; right bar of each pair) IgG deposition. FIG. 75H shows the percent of 42 patients with LN having negative (“0”; left bar of each pair) or positive (“>0”; right bar of each pair) IgM deposition. In (FIGS. 75A-C) significant differences in expected and observed frequencies between coral, the “least abnormal” LN endotype, and all other clusters (denoted with asterisk above bars) for (FIG. 75A) proliferative LN, (FIG. 75B) positive IgA deposition, and (FIG. 75C) active SLEDAI were identified by Chi Square Test. The likelihood of having proliferative LN in the coral cluster was not significantly different than the other clusters. The likelihood (odds ratio) of having positive IgA deposition in the coral cluster is 0.43 (p<0.0001) as compared to the other three clusters. The likelihood (odds ratio) of having active SLE (SLEDAI≥6) in the coral cluster is 0.06 (p<0.01) as compared to the other three clusters. In FIGS. 75A-75C significant associations between the categorical variables and all clusters (denoted with asterisks on the y-axis) were identified using Chi Square Test of Independence. In FIG. 75A only the yellow group had mesangial patients. In (FIGS. 75D-E) significant differences in mean of the renal activity or renal chronicity index between the coral cluster and each other cluster was assessed by Brown-Forsythe and Welch ANOVA with Dunnett's T3 multiple comparisons. **, p<0.01, ****, p<0.0001.

FIGS. 76A-I show comparison of the molecular endotypes, as shown in FIGS. 74A and B, for GSVA enrichment of signatures for TCA cycle (FIG. 76A), Oxidative Phosphorylation (FIG. 76B), Fatty Acid Beta Oxidation (FIG. 76C), Kidney Cell (FIG. 76D), Podocyte (FIG. 76E), Proximal Tubule (FIG. 76F), Monocyte/Myeloid Cell (FIG. 76G), T cell (FIG. 76H), and B cell (FIG. 76I). Significant differences in mean GSVA enrichment score between Coral, the “least abnormal” LN endotype, and each other cluster were assessed by Brown-Forsythe and Welch ANOVA with Dunnett's T3 multiple comparisons. *, p<0.05, ***, p<0.001, ****, p<0.0001.

FIG. 77 shows clustering of GSVA enrichment scores into the four kidney-derived molecular clusters, using informative cellular and pathway signatures (Tables 25-1 to 25-32) in paired blood from patients with LN (BH11201). The columns represent individual patients that are grouped into four clusters (Coral, Yellow, Purple, Black). The rows represent gene modules indicative of immune/inflammatory cells and cellular pathways/processes. For FIG. 77 the molecular features (e.g., modules) listed from top to bottom (on the left vertical axis) are IFN, Immunoproteasome, Plasma Cell, IG Chains, Cell Cycle, SNOR Low UP, IL1 Pathway, Inflammasome, Inhibitory Macrophage, Inflammatory Cytokines, Anti-inflammation, TNF, Monocyte, Neutrophil, Granulocyte, LDG, Dendritic Cell, pDC, TCRD, NK Cell, MHCII, B Cell, gd T Cell, Anergic/activated T Cell, Oxidative Phosphorylation, Unfolded Protein, TCRAJ, T Cell, TCRA, TCRB, IL23 Complex and Treg

FIGS. 78A-L show analysis of paired blood of patients with LN demonstrates cluster-specific enrichment of LDG, T cell, dendritic cell, and glucocorticoid signatures. GSVA enrichment of (FIG. 78A) LDG, (FIG. 78B) T cell, (FIG. 78C) TCRA, (FIG. 78D) TCRAJ, (FIG. 78E) TCRB, (FIG. 78F) anergic/activated T cell, (FIG. 78G) dendritic cell, (FIG. 78H) glucocorticoid, (FIG. 781) interferon (IFN), (FIG. 78J) monocyte, (FIG. 78K) B cell, and (FIG. 78L) plasma cell signatures in the blood of 71 patients with LN (BH11201) are shown. X-axis clusters denote the cluster to which the sample belongs based upon analysis of paired kidney gene expression. Significant differences in enrichment of gene signatures between each cluster and Coral was assessed by Brown-Forsythe and Welch ANOVA with Dunnett's T3 multiple comparisons. *, p<0.05, **, p<0.01, ***, p<0.001. The glucocorticoid signature is derived from Northcott et al. (2).

FIGS. 79A-I show the LDG and T cell signatures are consistently correlated with the glucocorticoid signature in the blood of patients with LN, whereas the dendritic cell signature is not. Linear regression of the glucocorticoid signature with the LDG, T cell, and dendritic cell signatures in the blood of patients with lupus nephritis for (FIGS. 79A-C) BH11201 (n=71), (FIGS. 79D-F) GSE49454 (n=19), and (FIGS. 79G-J) GSE99967 (n=28) is shown. The glucocorticoid signature is derived from Northcott et al. (2). In each of FIGS. 79-I the glucocorticoid signature is shown in the x-axis.

FIGS. 80A-B show the expression of erythropoietin (EPO) or a recombinant human erythropoietin (rHuEPO) signature in the blood of patients with LN is not associated with the molecular endotypes LN. (a) Log2 expression of EPO in the paired blood of 71 patients with LN. (b) GSVA enrichment of the rHuEPO signature in the paired blood of 71 patients with LN. The rHuEPO signature was derived from Wang et al (3), where differentially expressed genes were measured after administration of rHuEPO, and nine of the genes that were consistently expressed after rHuEPO administration comprised the signature.

FIGS. 81A-B show unsupervised gene co-expression network analysis defines molecular profiles of NZM2328 mice correlated with disease severity. K-means clustering (k=4) of NZM2328 CTL, AGN, TGN, and CGN mouse glomeruli (FIG. 81A) and TI (FIG. 81B) based on GSVA enrichment scores of MEGENA modules. The optimal number of module clusters was defined by the silhouette method and annotated by gene overlap with curated immunologic signatures and GO terms. Heatmap visualizations depict positive to negative GSVA scores on a red to blue gradient and positive to negative correlations between GSVA scores and disease classification on a gold to blue gradient. For FIGS. 81A-B, clusters (vertical) shown from left to right are coral, maroon, green and blue.

FIGS. 82A-E show gene signature-based clustering of GN stages in NZM2328 mice translates to human LN patients. (FIGS. 82A-B) K-means clustering (k=4) of NZM2328 CTL, AGN, TGN, and CGN mouse glomeruli (FIG. 82A) and TI (FIG. 82B) based on GSVA enrichment scores of selected immune cell, kidney cell, and metabolic pathway gene sets. (FIGS. 82C-E) K-means clustering (k=4) of microdissected glomeruli (FIG. 82C), TI (FIG. 82D), and whole kidney (FIG. 82E) from human LN patients based on GSVA score from human orthologs of the mouse gene sets used in FIGS. 82A&B. Heatmap visualizations depict positive to negative GSVA scores on a red to blue gradient and positive to negative correlations between GSVA scores and disease classification on a gold to blue gradient. For FIGS. 82A-E, clusters (vertical) shown from left to right are coral, maroon, green and blue.

FIG. 83 shows expression of Cgnz1 locus genes in the TI of NZM2328 and R27 AGN mice. Log2 expression values of genes in the Cgnz1 risk locus from the TI of NZM2328 and R27 CTL and AGN mice. Statistical significance was evaluated separately for NZM2328 CTL vs AGN and R27 CTL vs AGN comparisons. For each gene the bars from left to right show expression in NZM2328 control, NZM2328 AGN, R27 control and R27 AGN mice.

FIG. 84 shows gene signature-based clustering of IFNα-NZB mouse kidneys. K-means clustering (k=3) of IFNα-NZB mice over time after IFNα treatment based on GSVA enrichment scores of selected immune cell, kidney cell, and metabolic pathway gene sets. Heatmap visualizations depict positive to negative GSVA scores on a red to blue gradient and positive to negative correlations between GSVA scores and disease classification on a gold to blue gradient. For FIG. 84, clusters (vertical) shown from left to right are coral, maroon, green and blue.

FIGS. 85A-B show NZM2328 mouse MEGENA module-based clustering of human LN kidneys. K-means clustering (k=4) of whole kidney samples from human LN patients based on GSVA scores from human orthologs of the MEGENA modules from NZM2328 mouse microdissected glomeruli (A) and TI (B). The optimal number of module clusters was defined by the silhouette method and annotated by gene overlap with curated immunologic signatures and GO terms. Heatmap visualizations depict positive to negative GSVA scores on a red to blue gradient and positive to negative correlations between GSVA scores and disease classification on a gold to blue gradient. For FIGS. 85A-B, clusters (vertical) shown from left to right are coral, maroon, green and blue.

### INCLUDED EMBODIMENTS

1. A method for assessing a lupus nephritis disease state of a patient, the method comprising: analyzing a data set comprising or derived from gene expression measurement data of at least 2 genes or human orthologs thereof selected from the genes listed in Tables 19-1 to 19-36, Tables 19A-1 to 19A-36, Table 20, Table 21, Table 22, Tables 23-1 to 23-28, Tables 25-1 to 25-32, Tables 26-1 to 26-60, Tables 27-1 to 27-48, and Tables 28-1 to 28-22 in a biological sample from the patient, to classify the lupus nephritis disease state of the patient.
2. The method of embodiment 1, wherein the lupus nephritis disease state of the patient is classified as acute lupus nephritis, transitional lupus nephritis, chronic lupus nephritis, or absence of lupus nephritis.
3. The method of embodiment 1 or 2, wherein the data set comprises or is derived from gene expression measurement data of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 450, 500, 550, 600, 650, 700, 750, 850, 900, 950, 1000, 1050, 1100, 1150, 1200, 1250, 1300, 1350, 1400, 1450, 1500, 1550, 1600, 1700, 1800, 1900, or 2000 genes, selected from the genes listed in Tables 19-1 to 19-36, Tables 19A-1 to 19A-36, Table 20, Table 21, Table 22, Tables 23-1 to 23-28, Tables 23-1 to 23-28, Tables 26-1 to 26-60, Tables 27-1 to 27-48, and Tables 28-1 to 28-22 in the biological sample from the patient.
4. The method of any one of embodiments 1 to 3, wherein the genes or human orthologs thereof are selected from the genes listed in Tables 19-1 to 19-36.
5. The method of any one of embodiments 1 to 3, wherein the genes or human orthologs thereof are selected from the genes listed in Table 20.
6. The method of any one of embodiments 1 to 3, wherein the genes or human orthologs thereof are selected from the genes listed in Table 21.
7. The method of any one of embodiments 1 to 3, wherein the genes or human orthologs thereof are selected from the genes listed in Table 22.
8. The method of any one of embodiments 1 to 3, wherein the genes are selected from the genes listed in Tables 23-1 to 23-28.
9. The method of any one of embodiments 1 to 3, wherein the genes are selected from the genes listed in Tables 25-1 to 25-32.
10. The method of any one of embodiments 1 to 3, wherein the genes are selected from the genes listed in Tables 26-1 to 26-60.
11. The method of any one of embodiments 1 to 3, wherein the genes are selected from the genes listed in Tables 27-1 to 28-48.
12. The method of any one of embodiments 1 to 3, wherein the genes are selected from the genes listed in Tables 28-1 to 28-22.
13. The method of any one of embodiments 1 to 12, wherein the data set comprises or is derived from gene expression measurement data of at least 2 to all, or any value or range there between, genes or human orthologs thereof selected from the genes listed in each of one or more Tables selected from Tables 19-1 to 19-36, Tables 19A-1 to 19A-36, Table 20, Table 21, Table 22, Tables 23-1 to 23-28, Tables 25-1 to 25-32, Tables 26-1 to 26-60, Tables 27-1 to 27-48, and Tables 28-1 to 28-22 in the biological sample from the patient, wherein a different or identical number of genes are selected from the genes listed in each selected table.
14. The method of any one of embodiments 1 to 4 and 13, wherein the one or more Tables are selected from Tables 19-1 to 19-36.
15. The method of embodiment 14, wherein the one or more Tables comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 Tables selected from Tables 19-1 to 19-36.
16. The method of embodiment 14 to 15, wherein the selected Tables are Tables 19-1 to 19-36.
17. The method of any one of embodiments 1 to 3, 8 and 13, wherein the one or more Tables are selected from Tables 23-1 to 23-28.
18. The method of embodiment 17, wherein the one or more Tables comprise at least 2, 3, 4, 5, 6, 7, 8, 9, 10, 11, 12, 13, 14, 15, 16, 17, 18, 19, 20, 21, 22, 23, 24, 25, 26, 27, or 28 Tables selected from Tables 23-1 to 23-28.
19. The method of embodiment 17 or 18, wherein the selected Tables are Tables 23-1 to 23-28.
20. The method of any one of embodiments 1 to 3, 9 and 13, wherein the one or more Tables are selected from Tables 25-1 to 25-32.
21. The method of embodiment 20, wherein the one or more Tables comprise at least 2, 3, 4, 5, 6, 7, 8, 9, 10, 11, 12, 13, 14, 15, 16, 17, 18, 19, 20, 21, 22, 23, 24, 25, 26, 27, or 28 Tables selected from Tables 25-1 to 25-32.
22. The method of embodiment 20 or 21, wherein the selected Tables are Tables 25-1 to 25-32.
23. The method of any one of embodiments 1 to 3, 10 and 13, wherein the one or more Tables are selected from Tables 26-1 to 26-60.
24. The method of embodiment 23, wherein the one or more Tables comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Tables selected from Tables 26-1 to 26-60.
25. The method of embodiments 23 or 24, wherein the selected Tables are 26-1 to 26-60.
26. The method of any one of embodiments 1 to 3, 11 and 13, wherein the one or more Tables are selected from Tables 27-1 to 27-48.
27. The method of embodiment 26, wherein the one or more Tables comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 Tables selected from Tables 27-1 to 27-48.
28. The method of embodiments 26 or 27, wherein the selected Tables are 27-1 to 27-48.
29. The method of any one of embodiments 1 to 3, 12 and 13, wherein the one or more Tables are selected from Tables 28-1 to 28-22.
30. The method of embodiment 29, wherein the one or more Tables comprise at least 2, 3, 4, 5, 6, 7, 8, 9, 10, 11, 12, 13, 14, 15, 16, 17, 18, 19, 20, or 22 Tables selected from Tables 28-1 to 28-22.
31. The method of embodiment 29 or 30, wherein the selected Tables are Tables 28-1 to 28-22.
32. The method of any one of embodiments 1 to 31, wherein the lupus nephritis disease state of the patient is classified with an accuracy of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
33. The method of any one of embodiments 1 to 32, wherein the lupus nephritis disease state of the patient is classified with a sensitivity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
34. The method of any one of embodiments 1 to 33, wherein the lupus nephritis disease state of the patient is classified with a specificity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
35. The method of any one of embodiments 1 to 34, wherein the lupus nephritis disease state of the patient is classified with a positive predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
36. The method of any one of embodiments 1 to 35, wherein the lupus nephritis disease state of the patient is classified with a negative predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.
37. The method of any one of embodiments 1 to 36, wherein the lupus nephritis disease state of the patient is classified with a Receiver operating characteristic (ROC) curve having an Area-Under-Curve (AUC) of at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.
38. The method of any one of embodiments 1 to 37, wherein the data set is derived from the gene expression measurement data using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, Z-score, log 2 expression analysis, or any combination thereof.
39. The method of any one of embodiments 1 to 38, wherein the data set is derived from the gene expression measurement data using GSVA.
40. The method of embodiment 39, wherein the data set comprises one or more GSVA scores of the patient, wherein the one or more GSVA scores are generated based on one or more Tables selected from Tables 19-1 to 19-36, Tables 19A-1 to 19A-36, Tables 23-1 to 23-28, Tables 25-1 to 25-32, Tables 26-1 to 26-60, Tables 27-1 to 27-48, and Tables 28-1 to 28-22, wherein for each selected Table, at least one GSVA score of the patient is generated based on enrichment of expression of at least 2 genes or human orthologs thereof listed in the selected Table, and wherein the one or more GSVA scores comprise each generated GSVA score.
41. The method of embodiment 40, wherein the one or more Tables are selected from Tables 19-1 to 19-36.
42. The method of embodiments 40 or 41, wherein the one or more Tables comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 Tables selected from Tables 19-1 to 19-36.
43. The method of any one of embodiments 40 to 42, wherein the selected tables comprise Tables 19-1 to 19-36.
44. The method of embodiment 40, wherein the one or more Tables are selected from Tables 23-1 to 23-28.
45. The method of embodiment 40 or 44, wherein the one or more Tables comprise at least 2, 3, 4, 5, 6, 7, 8, 9, 10, 11, 12, 13, 14, 15, 16, 17, 18, 19, 20, 21, 22, 23, 24, 25, 26, 27, or 28 Tables selected from Tables 23-1 to 23-28.
46. The method of embodiment 40, 44, or 45, wherein the selected tables comprise Tables 23-1 to 23-28.
47. The method of embodiment 40, wherein the one or more Tables are selected from Tables 25-1 to 25-32.
48. The method of embodiment 40 or 47, wherein the one or more Tables comprise at least 2, 3, 4, 5, 6, 7, 8, 9, 10, 11, 12, 13, 14, 15, 16, 17, 18, 19, 20, 21, 22, 23, 24, 25, 26, 27, or 28 Tables selected from Tables 25-1 to 25-32.
49. The method of embodiment 40, 47, or 48, wherein the selected tables comprise Tables 25-1 to 25-32.
50. The method of embodiment 50, wherein the one or more Tables are selected from Tables 26-1 to 26-60.
51. The method of embodiment 40 or 50, wherein the one or more Tables comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Tables selected from Tables 26-1 to 26-60.
52. The method of embodiment 40, 50, or 51, wherein the selected tables comprise Tables 26-1 to 26-60.
53. The method of embodiment 40, wherein the one or more Tables are selected from Tables 27-1 to 27-48.
54. The method of embodiment 40 or 53, wherein the one or more Tables comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 Tables selected from Tables 27-1 to 27-48.
55. The method of embodiment 40, 53, or 54, wherein the selected tables comprise Tables 27-1 to 27-22.
56. The method of embodiment 40, wherein the one or more Tables are selected from Tables 28-1 to 28-22.
57. The method of embodiment 40 or 56, wherein the one or more Tables comprise at least 2, 3, 4, 5, 6, 7, 8, 9, 10, 11, 12, 13, 14, 15, 16, 17, 18, 19, 20, 21, or 22 Tables selected from Tables 28-1 to 28-22.
58. The method of embodiment 40, 56, or 57, wherein the selected tables comprise Tables 28-1 to 28-22.
59. The method of any one of embodiments 40 to 58, wherein independently for each selected Table, the at least one GSVA score of the patient is generated based on enrichment of expression of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295 or all genes selected from the genes listed in the respective Table.
60. The method of any one of embodiments 1 to 59, wherein the analyzing the data set comprises providing the data set as an input to a trained machine-learning model to classify the lupus nephritis disease state of the patient, wherein the trained machine-learning model generates an inference indicative of the lupus nephritis disease state of the patient based at least on the data set.
61. The method of embodiment 60, wherein the data set comprises the one or more GSVA scores of the patient, and the trained machine-learning model generates the inference based at least on the one or more GSVA scores.
62. The method of embodiment 60 or 61, wherein the method further comprises receiving, as an output of the trained machine-learning model, the inference; and/or electronically outputting a report indicating the lupus nephritis disease state of the patient.
63. The method of any one of embodiments 60 to 62, wherein the machine-learning model is trained using linear regression, logistic regression, Ridge regression, Lasso regression, elastic net (EN) regression, support vector machine (SVM), gradient boosted machine (GBM), k nearest neighbors (kNN), generalized linear model (GLM), naïve Bayes (NB) classifier, neural network, Random Forest (RF), deep learning algorithm, linear discriminant analysis (LDA), decision tree learning (DTREE), adaptive boosting (ADB), Classification and Regression Tree (CART), hierarchical clustering, or any combination thereof.
64. The method of any one of embodiments 1 to 63, wherein the lupus nephritis disease state of the patient is classified based on a lupus nephritis disease risk score generated from the data set.
65. The method of embodiment 64, wherein the lupus nephritis disease risk score is generated based on the one or more GSVA scores of the patient.
66. The method of any one of embodiments 1 to 65, wherein the patient is at elevated risk of having lupus.
67. The method of any one of embodiments 1 to 66, wherein the patient is suspected of having lupus.
68. The method of any one of embodiments 1 to 67, wherein the patient is asymptomatic for lupus.
69. The method of any one of embodiments 1 to 68, wherein the patient has lupus.
70. The method of any one of embodiments 1 to 69, wherein the patient is at elevated risk of having lupus nephritis.
71. The method of any one of embodiments 1 to 70, wherein the patient is suspected of having lupus nephritis.
72. The method of any one of embodiments 1 to 71, wherein the patient is asymptomatic for lupus nephritis.
73. The method of any one of embodiments 1 to 72, wherein the patient has lupus nephritis.
74. The method of any one of embodiments 1 to 73, further comprising identifying, selecting, recommending and/or administering a treatment to the patient based at least in part on the classification of the lupus nephritis disease state of the patient.
75. The method of embodiment 74, wherein the treatment is configured to treat lupus nephritis.
76. The method of embodiment 74, wherein the treatment is configured to reduce a severity of lupus nephritis.
77. The method of embodiment 74, wherein the treatment is configured to reduce a risk of having lupus nephritis.
78. The method of any one of embodiments 74 to 77, wherein the treatment comprises a pharmaceutical composition.
79. The method of any one of embodiments 1 to 78, wherein the biological sample comprises a kidney biopsy sample, a blood sample, isolated peripheral blood mononuclear cells (PBMCs), or any derivative thereof.
80. A method for validating a mouse model useful for identifying and/or characterizing a human disease, the method comprising:
   a) providing a gene set capable of classifying a mouse as having an endotype selected from two or more endotypes of the disease;
   b) determining human orthologs of the gene set;
   c) classifying a human patient as having an endotype selected from the two or more endotypes of the disease using the human orthologs; and
   d) using the human orthologs to classify the mouse model as having an endotype selected from the two or more endotypes of the disease,
wherein the endotype of a validated mouse model classified using the human orthologs corresponds to the human endotype of step (c) identified using the human

### DETAILED DESCRIPTION

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the term “Gini impurity” refers to a measure of how often a randomly chosen element from the set may be incorrectly labeled if it is randomly labeled according to the distribution of labels in the subset.

As used herein the term “lesion” refers to a potential disease lesion, e.g., a skin lesion potentially associated with and/or potentially directly resulting from lupus, psoriasis, atopic dermatitis, systemic sclerosis (scleroderma), or a combination thereof, as determined by one of skill in the art. In some embodiments, the lesion does not include a traumatic injury, e.g., a cut, scrape, scratch, burn, etc., and/or a skin affliction of any known origin not associated with a disease state indicated by the skin classification, e.g., contact dermatitis, a food allergy, and/or a drug reaction. In some embodiments, the skin lesion does not include a lesion that is not potentially associated with and/or potentially directly resulting from lupus, psoriasis, atopic dermatitis, systemic sclerosis (scleroderma), or a combination thereof.

Reference in the specification to “embodiments,” “certain embodiments,” “preferred embodiments,” “specific embodiments,” “some embodiments,” “an embodiment,” “one embodiment” or “other embodiments” mean that a particular feature, structure, or characteristic described in connection with the embodiments is included in at least some embodiments, but not necessarily all embodiments, of the present disclosure.

Many complex and multi-systematic diseases and conditions currently pose major diagnostic and therapeutic challenges. Despite the wealth of records from, for example, genetic, epigenetic, and gene expression data that has emerged in the past few years, physicians often still rely on clinical evaluation and laboratory tests, including measurement of autoantibodies and complement levels.

Successful relation of records (e.g., gene expression records) to a specific disease phenotype activity has been attempted, including efforts to identify individual genes that predicted subsequent flares, and through the determination of a discrete group of differentially expressed (DE) genes that may be found in a particular record. Despite these advances, however, no such approach is available with sufficient predictive value to utilize in evaluation and treatment.

As such, there is a need for a predictive tool for evaluating patient at both the chemical and cellular levels to advance personalized treatment. Data analytical techniques such as machine learning enable proper correlation between genetic records and phenotypes.

The machine learning models tested here provide the basis of personalized medicine. Integration of the methods herein with emerging high-throughput record sampling technologies may unlock the potential to develop a simple blood test to predict phenotypic activity. The disclosures herein may be generalized to predict other manifestations, such as organ involvement. A better understanding of the cellular processes that drive pathogenesis may eventually lead to customized therapeutic strategies based on records' unique patterns of cellular activation.

### Method of Identifying One or More Records Having a Specific Phenotype

One aspect disclosed herein is a method of identifying one or more records (e.g., raw gene expression data, whole gene expression data, blood gene expression data, or informative gene modules). The method may comprise receiving a plurality of first records, receiving a plurality of second records, receiving a plurality of third records, applying a machine learning algorithm to at least one first record and at least one second record to determine a classifier (e.g., a machine learning classifier), and applying the classifier to the plurality of third records. Applying the classifier to the plurality of third records may identify one or more third records associated with the specific phenotype. In some embodiments, applying a machine learning algorithm to the third data set comprises applying a machine learning algorithm to a plurality of unique third data sets.

### Records

The records may comprise, for example, raw gene expression data, whole gene expression data, blood gene expression data, informative gene modules, or any combination thereof. The records may be generated by Weighted Gene Co-expression Network Analysis (WGCNA). In some embodiments, at least one of the first records and the second records comprise nucleic acid sequencing data, transcriptome data, genome data, epigenome data, proteome data, metabolome data, virome data, methylome data, lipidomic data, lineage-ome data, nucleosomal occupancy data, a genetic variant, a gene fusion, an insertion or deletion (indel), or any combination thereof. In some embodiments, the first records and the second records are in different formats. In some embodiments, the first records and the second records are from different sources, different studies, or both.

In some embodiments each record is associated with a specific phenotype (e.g., a disease state, an organ involvement, or a medication response). Each first record may be associated with one or more of a plurality of phenotypes. The plurality of second records and the plurality of first records may be non-overlapping. The third records may be distinct from the plurality of first records, the plurality of second records, or both. The third records may comprise a plurality of unique third data sets.

The records may be received from the Gene Expression Omnibus (GEO, publicly available from the National Center for Biotechnology Information, e.g., on the website operated by National Library of Medicine, National Institutes of Health). The records may be associated with purified cell populations, whole blood gene expression, or both. A data set may comprise records comprising microarray, next-generation sequencing, and any other form of high-throughput functional genomic data known to those of skill in the art. The records received from a Gene Expression Omnibus source may comprise GSE10325, GSE26975, GSE38351, GSE39088, GSE45291, GSE49454, GSE72535, GSE52471, GSE81071, GSE109248, GSE100093, GSE120809, GSE117239, GSE117468, GSE130588, GSE58095, GSE95065, GSE121212, GSE137430, GSE157194, GSE130955, or any combination thereof. The records received from a Gene Expression Omnibus source may comprise GSE32583, GSE49898, GSE72410, GSE153021, GSE32591, GSE86423, GSE8642, or any combination thereof.

For example, as the most important genes may be involved in a number of functions other than interferon signaling, such RNA processing, ubiquitylation, and mitochondrial processes, these pathways may play important roles in directing, or at least be indicative of, phenotypic activity. CD4 T cells originally may contribute the most important modules. However, when the modules are de-duplicated, CD14 monocyte-derived modules prove important as unique genes expressed by CD14 monocytes in tandem with interferon genes may be informative in the study of cell-specific methods of pathogenesis.

### Phenotypes

In some embodiments, the phenotype comprises a disease state, an organ involvement a medication response, or any combination thereof. The disease state may comprise an active disease state, or an inactive disease state. At least one of the active disease state and the inactive disease state may be characterized by standard clinical composite outcome measures. The active disease state may comprise a Disease Activity Index of 6 or greater.

The disease may comprise an acute disease, a chronic disease, a clinical disease, a flare-up disease, a progressive disease, a refractory disease, a subclinical disease, or a terminal disease. The disease may comprise a localized disease, a disseminated disease, or a systemic disease. The disease may comprise an immune disease, a cancer, a genetic disease, a metabolic disease, an endocrine disease, a neurological disease, a musculoskeletal disease, or a psychiatric disease. The active disease state may comprise a Systemic Lupus Erythematosus Disease Activity Index (SLEDAI) of 6 or greater.

The organ involvement may comprise a possibly involved organ. The possibly involved organ may comprise bone, skin, hematopoietic system, spleen, liver, lung, mucosa, eye, ear, pituitary, or any combination thereof. The medication response may comprise an ultra-rapid metabolizer response, an extensive metabolizer response, an intermediate metabolizer response, or a poor metabolizer response. The ultra-rapid metabolizer response may refer to a record with substantially increased metabolic activity. The extensive metabolizer response may refer to a record with normal metabolic activity. The intermediate metabolizer response may refer to a record with reduced metabolic activity. The poor metabolizer response may refer to a record with little to no functional metabolic activity.

### Machine Learning and Classifiers

The classifiers described herein may be used in machine learning algorithms. A variety of machine learning classifiers exist, wherein each classifier produces a unique machine learning process and/or output. The machine learning algorithms may comprise a biased algorithm or an unbiased algorithm. The biased algorithm may comprise Gene Set Enrichment Analysis (GSVA) enrichment of phenotype-associated cell-specific modules. The unbiased approach may employ all available phenotypic data. The machine learning algorithm may comprise an elastic generalized linear model (GLM), a k-nearest neighbors classifier (KNN), a random forest (RF) classifier, or any combination thereof. GLM, KNN, and RF machine learning algorithms may be performed using the glmnet, caret, and random Forest R packages, respectively.

The random forest classifier is able to sort through the inherent heterogeneity of the plurality of records to identify one or more third records associated with the specific phenotype. In some embodiments, the classifier identifies said one or more third records associated with the specific phenotype with an accuracy of at least about 70%. The implementation of the random forest classifier herein enable a specific phenotype association sensitivity of 85% and a specific phenotype association specificity of 83%. Further classifier optimization, however, may yield improved results.

KNN may classify unknown samples based on their proximity to a set number K of known samples. K may be 5% of the size of the pluralities of first, second, and third records. Alternatively, K may be 1%, 2%, 3%, 4%, 5%, 6%, 7%, 8%, 9%, 10%, or any increment therein. A large K value may enable more precise calculations with less overall noise. Alternatively, the k-value may be determined through cross-validation by using an independent set of records to validate the K value. If the initial value of k is even, 1 may be added in order to avoid ties. RF may generate 500 decision trees which vote on the class of each sample. The Gini impurity index, a standard measure of misclassification error, correlates to the importance of such variables. In addition, pooled predictions may be assigned based on the average class probabilities across the three classifiers.

The GLM algorithm may carry out logistic regression with a tunable elastic penalty term to find a balance between an L1 (LASSO) and an L2 (ridge), whereby penalties facilitate variable selection in order to generate sparse solutions. Least Absolute Shrinkage and Selection Operator (LASSO) is a regularization feature selection technique to reduce overfitting in regression problems. Ridge regression employs a penalty term is to shrink the LASSO coefficient values. In some embodiments, the elastic generalized linear model classifier employs an elastic penalty of about 0.9, wherein the penalty is 90% lasso and 10% ridge. The elastic penalty may be 0.1, 0.2, 0.3, 0.4, 0.5, 0.6, 0.7, 0.8, 0.9, or any increments therein.

Records may be classified as active or inactive using two different methodologies: (1) a leave-one-study-out cross-validation approach or (2) a 10-fold cross-validation approach. GLM, KNN, and RF classifiers may be tasked with identifying active and inactive state records based on whole blood (WB) gene expression data and module enrichment data.

Supervised classification approaches using elastic generalized linear modeling, k-nearest neighbors, and random forest classifiers may be implemented. The trends in performance when cross-validating by one of the pluralities of records or cross-validating 10-fold display the potential advantages and disadvantages of diagnostic tests incorporating gene expression data or module enrichment. Cross-validating by one of the pluralities of records may be used to generalize 1-fold cross validation as a suboptimal scenario, whereas a 10-fold cross-validation is in fact more optimal. Although classification of active and inactive records from the pluralities of different records with 1-fold cross-validation may be suboptimal, module enrichment may be employed to smooth out much of the technical variation between data sets. 10-fold cross-validation may enable a more standardized diagnostic test. Although the plurality of second records and the plurality of first records are non-overlapping, the test set employs overlapping records to facilitate proper classification.

Furthermore, modules that may be negatively associated with phenotypic activity may be just as important in classification as positively associated modules. Further study of underrepresented categories of transcripts may enhance understanding and correlation of phenotypic activity.

Reduction of technical noise may improve classification. For example, RNA-Seq platforms, which produce transcript count records rather than probe intensity values, may display less technical variation across records if all samples are processed in the same way.

The strong performance of the random forest classifier indicates that nonlinear, decision tree-based methods of classification may be ideal because decision trees ask questions about new records sequentially and adaptively. Random forest does not apply a one-size-fits-all approach to each of the different types of records to allow for classification of records whose expression patterns make them a minority within their phenotype. As such, active records that do not resemble the majority of active records still have a strong chance of being properly classified by random forest. By contrast other methods may approach variables from new records all at once.

### Filtering

In some embodiments, the method further comprises filtering the first records, the second records, or both. In some embodiments, the filtering comprises normalizing, variance correction, removing outliers, removing background noise, removing data without annotation data, scaling, Weighted Gene Co-expression Network Analysis, enrichment analysis, dimensionality reduction, or any combination thereof.

In some embodiments, the normalizing is performed by Robust Multi-Array Analysis (RMA), Guanine Cytosine Robust Multi-Array Analysis (GCRMA), Linear Models for Microarray Data, variance stabilizing transformation (VST), normal-exponential quantile correction (NEQC), or any combination thereof. RMA may summarize the perfect matches through a median polish algorithm, quantile normalization, or both. Variance-stabilizing transformation may simplify considerations in graphical exploratory data analysis, allow the application of simple regression-based or analysis of variance techniques, or both. Normalized expression values may be variance corrected using local empirical Bayesian shrinkage, and DE may be assessed using the Linear Models for Microarray Data (LIMMA) package. Resulting p-values may be adjusted for multiple hypothesis testing using the Benjamini-Hochberg correction, which resulted in a false discovery rate (FDR). Significant genes within each study may be filtered to retain DE genes with an FDR<0.2, which may be considered statistically significant. The FDR may be selected a priori to diminish the number of genes that may be excluded as false negatives.

In some embodiments, the variance correction comprises employing a local empirical Bayesian shrinkage, adjusting the p-values for multiple hypothesis testing using the Benjamini-Hochberg correction, removing all data with a false discovery rate of less than 0.2, or any combination thereof. The Benjamini-Hochberg procedure may decrease the false discovery rate caused by incorrectly rejecting the true null hypotheses control for small p-values.

In some embodiments, the Weighted Gene Co-expression Network Analysis comprises calculating a topology matrix, clustering the data based on the topology matrix, correlating module eigenvalues for traits on a linear scale by Pearson correlation for nonparametric traits by Spearman correlation and for dichotomous traits by point-biserial correlation or t-test, or both. A topology matrix may specify the connections between vertices in directed multigraph.

Log2-normalized microarray expression values from purified CD4, CD14, CD19, CD33, and low density granulocyte (LDG) populations may be used as input to WGCNA to conduct an unsupervised clustering analysis, resulting in co-expression “modules,” or groups of densely interconnected genes which may correspond to comparably regulated biologic pathways. For each experiment, an approximately scale-free topology matrix (TOM) may be first calculated to encode the network strength between probes. Probes may be clustered into WGCNA modules based on TOM distances. Resultant dendrograms of correlation networks may be trimmed to isolate individual modular groups of probes by partitioning around medoids and labeled using color assignments based on module size. Expression profiles of genes within modules may be summarized by a module eigengene (ME), which may be analogous to the module's first principal component. MEs act as characteristic expression values for their respective modules and may be correlated with sample traits such as SLEDAI or cell type by Pearson correlation for continuous or semi-continuous traits and by point-biserial correlation for dichotomous traits.

WGCNA modules from CD4, CD14, CD19, and CD33 cells may be tested for correlation to SLEDAI. Plasma cell modules may be generated by differential expression analysis and not WGCNA, but may be included because of the established importance of plasma cells in SLE pathogenesis.

Removing the outliers may be performed by statistical analysis using R and relevant Bioconductor packages. Non-normalized arrays may be inspected for visual artifacts or poor hybridization using Affy QC plots. Principal Component Analysis (PCA) plots may be used to inspect the raw data files for outliers. Data sets culled of outliers may be cleaned of background noise and normalized using RMA, GCRMA, or NEQC where appropriate. Data sets may be then filtered to remove probes with low intensity values and probes without gene annotation data. WB gene expression data sets may be filtered to only include genes that passed quality control in all data sets. Differential expression (DE) analysis and WGCNA may then be carried out on data sets. WB gene expression data sets may then be further processed before machine learning analysis. WB gene expression values may be centered and scaled to have zero-mean and unit-variance within each data set and the standardized expression values from each data set may be joined for classification.

The GSVA-R package may be used as a non-parametric method for estimating the variation of pre-defined gene sets in WB gene expression data sets. Standardized expression values from WB data sets may be used to test for enrichment of cell-specific WGCNA gene modules using the Single-sample Gene Set Enrichment Analysis (ssGSEA) method, which scores single samples in isolation and may be thus shielded from technical variation within and among data sets. Statistical analysis of GSVA enrichment scores may be performed by Spearman correlation or Welch's unequal variances t-test, where appropriate. GSVA may be performed on three WB datasets using 25 WGCNA modules made from purified cells with correlation or published relationship to SLEDAI.

Patterns of enrichment of WGCNA modules that are derived from isolated cell populations of WB that are correlated to the phenotype may be more useful than gene expression across the pluralities of records to identify active versus inactive state records. To characterize the relationships between gene signatures from various records and phenotypic activity, WGCNA may be used to generate co-expression gene modules from purified populations of cells from records with an active disease state. Such records may be subsequently tested for enrichment in whole blood of other records. WGCNA analysis of leukocyte subsets may result in several gene modules with significant Pearson correlations to SLEDAI (all |r|>0.47, p<0.05). CD4, CD14, CD19, and CD33 cells with 3, 6, 8, and 4 significant modules, respectively. Two low-density granulocyte (LDG) modules may be created by performing WGCNA analysis of LDGs along with either neutrophils or HC neutrophils and merging the modules most strongly expressed by LDGs Two plasma cell (PC) modules may be created by using the most increased and decreased transcripts of isolated plasma cells compared to naïve and memory B cells.

Gene Ontology (GO) analysis of the genes within each of the record indicates that that some processes, such as those related to interferon signaling, RNA transcription, and protein translation, may be shared among cell types, whereas other processes may be unique to certain cell types and may be used to better classification of records.

GSVA enrichment may be performed using the 25 cell-specific gene modules in WB from 156 records (82 active, 74 inactive). Of the 25 cell-specific modules, 12 had enrichment scores with significant Spearman correlations to SLEDAI (p<0.05), and 14 had enrichment scores with significant differences between active and inactive state records by Welch's unequal variances t-test (p<0.05). Notably, each cell type produced at least one module with a significant correlation to SLEDAI in WB and at least one module with a significant difference in enrichment scores between active and inactive records, demonstrating a relationship between phenotypic activity in specific cellular subsets and overall phenotypic activity in WB. However, as the Spearman's rho values ranged from −0.40 to +0.36, no one module may have a substantial predictive value. Furthermore, the effect sizes as measured by Cohen's d when testing active versus inactive enrichment scores ranged from −0.85 to +0.79. The CD4 Floralwhite and Orangered4 modules, which had the largest positive and negative effect sizes, respectively, showed a high degree of overlap in the enrichment scores of active and inactive records, where error bars indicate mean±standard deviation. WB may be unable to fully separate active records from inactive records.

Analysis of individual phenotypic activity associated peripheral cellular subset gene modules may not be sufficient to predict phenotypic activity in unrelated WB data sets, since no single module from any cell type may be able to separate active from inactive state records. Although no single module had a sufficiently high predictive value, many cell-specific gene modules may be combined and optimized to predict phenotypes of active records. Moreover, the results emphasized the need for more advanced analysis to employ gene expression analysis to predict phenotypic activity.

### Performance and Accuracy

When training and testing sets are formed by holding out entire data sets, machine learning algorithms using raw gene expression data had an average classification accuracy of only 53 percent. However, converting this gene expression data to module enrichment improved classification accuracy to 71 percent. When training and testing sets are formed by mixing records from the three data sets, module enrichment remained at a 70 percent classification accuracy. However, classification accuracy using raw gene expression increased to a mean of 79 percent. The best overall performance came from the random forest classifier, which had a predictive accuracy of 84 percent.

The performance of each machine learning algorithm may be determined by evaluating 2 different forms of cross-validation. A random 10-fold cross-validation may randomly assign each record to one of 10 groups. A leave-one-study-out cross-validation may determine the effects of systematic technical differences among data sets on classification performance. For each pass of cross-validation, one fold or study may be held out as a test set, whereby the classifiers are trained on the remaining data. Accuracy may be assessed as the proportion of records correctly classified across all testing folds. Performance metrics such as sensitivity and specificity may be assessed after cross-validation by agglomerating class probabilities and assignments from each fold or study. Receiver Operating Characteristic (ROC) curves may be generated using the pROC R package.

In almost all cases, the random forest classifier outperformed the GLM and KNN classifiers, although the results may be not significantly different when assessed by testing for equality of proportions (p>0.05). Pooled predictions based on the class probabilities from the three classifiers may not improve overall performance.

When cross-validating by study, the use of expression values may achieve an accuracy of only 53 percent, which is consistent with the findings that gene expression values may provide less value towards classifying unfamiliar records. When the training records and test records are greatly heterogeneous, the classifiers learning patterns may be less helpful for classifying test records. Remarkably, the use of module enrichment scores improved accuracy to approximately 70 percent.

The 10-fold cross-validation with raw gene expression values may result in better performance compared to the leave-one-study-out cross-validation. This increase in performance may be attributed to the presence of records from all plurality of first, second, and third records in both the training and test sets. In this case, the classifiers may learn patterns inherent to each set of records. In this circumstance, the random forest classifier may be the strongest performer with 84% accuracy (85% sensitivity, 83% specificity), whereby the ROC curve demonstrates an excellent tradeoff between recall and fall-out. The performance of module enrichment, however may not be substantially different between 10-fold cross-validation and leave-one-study-out cross-validation.

Overall, in a study-by-study approach (leave-one-study-out cross-validation), module enrichment may be more successful than raw gene expression. Importantly, when using the 10-fold cross-validation approach, raw gene expression may outperform module enrichment. Thus, phenotypic activity classification based on raw gene expression may be sensitive to technical variability, whereas classification based on module enrichment may cope better with variation among data sets.

The variable importance of Random forest provides insight into directors of the identification of phenotypic activity, random forest classifiers may be trained on all records from each of the plurality of records in order to identify the most important genes and modules as determined by mean decrease in the Gini impurity, a measure of misclassification error.

The most important genes and modules identified a wide array of cell types and biological functions. The most important genes encompass such diverse functions as interferon signaling, pattern recognition receptor signaling, and control of survival and proliferation. Notably, the most influential modules may be skewed away from B cell-derived modules and towards T cell- and myeloid cell-derived modules. As some of these modules had overlapping genes, the variable importance experiment may be repeated with modules that may be first scrubbed of any genes that appeared in more than one module before GSVA enrichment scoring. The relative variable importance scores of the de-duplicated modules correlated strongly with those of the original modules (Spearman's rho=0.73, p=5.18E-5), indicating that module behavior may be partly driven by the overlapping genes but strongly driven by unique genes. Variable importance of top 25 individual genes. LDG: low-density granulocyte; PC: plasma cell.

CD4_Floralwhite and CD14_Yellow, two interferon-related modules which maintained high importance after deduplication, may be further analyzed to study the effect of unique genes on module importance. Gene lists may be tested for statistical overrepresentation of Gene Ontology biological process terms with FDR correction on pantherdb.org. CD4_Floralwhite did not show any significant enrichment, but CD14_Yellow, which had the highest importance after deduplication, may be highly enriched for genes with the “Immune Effector Process” designation (26/77 genes, FDR=9.38E-11 by Fisher's exact test). This suggests that CD14+ monocytes express unique genes that may play important roles in the initiation of phenotypic activity.

Several important findings on the topic of gene expression heterogeneity within and across data sets have been elucidated by this study. First, DE analysis of active vs inactive records may be insufficient for proper classification of phenotypic activity, as systematic differences between data sets render conventional bioinformatics techniques largely non-generalizable.

Further, WGCNA modules created from the cellular components of WB and correlated to SLEDAI phenotypic activity may improve classification of phenotypic activity in records. The use of cell-specific gene modules based on a priori knowledge about their relevance to disease fared slightly better than raw gene expression, as it generated informative enrichment patterns, and many of the modules maintained significant correlations with SLEDAI in WB. However, these enrichment scores failed to completely separate active records from inactive records by hierarchical clustering.

### Method Characterization

Conventional bioinformatics approaches do not satisfactorily identify one or more records having a specific phenotype. DE analysis of a plurality of first records, a plurality of second records, and a plurality of third records having an active disease state and a non-active disease state displayed the major differences and heterogeneity. First, the 100 most significant DE genes by FDR in the plurality of first, second, and third records may be used to carry out hierarchical clustering of active and inactive disease state records. Active disease state records are clearly separated from inactive records, but only partially separated from inactive records.

Out of 6,640 unique DE genes from the three pluralities of records, 5,170 genes are unique to one of the plurality of records, 1,234 are shared by two of the plurality of records, and 36 are shared by all three of the plurality of records. There is minimal overlap of the 100 most significant genes by FDR in each of the pluralities of records. The only overlaps among the top 100 DE genes in each study by FDR are: TWY3 and EHBP1, shared between the plurality of first records and the plurality of third records; and LZIC, shared between the plurality of first records and plurality of second records. Furthermore, the fold change distributions of the 100 most significant DE genes in each of the pluralities of records varied considerably. In the plurality of first records, 94 of the 100 most significant genes are downregulated in active disease state records; in the plurality of second records, all of the top 100 genes are upregulated in active disease state records; and in the plurality of third records, the top 100 genes are more evenly distributed (41 up, 59 down). Orange bars denote active state records, wherein black bars denote inactive state records

The plurality of first, second, and third records may represent different populations and may be collected on different microarray platforms. The lack of commonality among the genes most descriptive of active state records and inactive state records in each of the pluralities of records casts doubt on whether active and inactive states from the different pluralities of records may be easily determined using conventional techniques.

Records from the pluralities of first, second, and third records may then be joined to evaluate whether unsupervised techniques may separate active state records from inactive state records. Hierarchical clustering on the 297 unique most significant DE genes by FDR showed considerable heterogeneity, and active records and inactive records did not consistently separate, per the heat map of the top 100 DE genes by FDR from each of the pluralities of records (combined total of 297 unique genes from the plurality of first, second, and third records) expressed in all records. As such, conventional techniques failed to identify active records, highlighting the need for more advanced algorithms.

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing-based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing device, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for identifying one or more records having a specific phenotype. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### Biological Data Analysis

The present disclosure provides systems and methods to perform data analysis using drug or target scoring algorithms and/or big data analysis tools. In various aspects, such drug or target scoring algorithms and/or big data analysis tools may be used to perform analysis of data sets including, for example, mRNA gene expression or transcriptome data, DNA genomic data, proteomic data, metabolomic data, other types of “-omic” data, or a combination thereof.

In an aspect, the present disclosure provides a computer-implemented method for assessing a condition of a subject, comprising: (a) receiving a dataset of a biological sample of the subject; (b) selecting one or more data analysis tools, wherein the one or more data analysis tools comprise an analysis tool selected from the group consisting of: a BIG-C™ big data analysis tool, an I_Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool; (c) processing the dataset using the one or more data analysis tools to generate a data signature of the biological sample of the subject; and (d) based at least in part on the data signature generated in (c), assessing the condition of the subject.

In some embodiments, the dataset comprises mRNA gene expression or transcriptome data, DNA genomic data, proteomic data, metabolomic data, or a combination thereof. In some embodiments, the biological sample comprises a whole blood (WB) sample, a PBMC sample, a tissue sample, a cell sample, or any derivative thereof. In some embodiments, assessing the condition of the subject comprises identifying a disease or disorder of the subject.

In some embodiments, the method further comprises identifying a disease or disorder of the subject at a sensitivity or specificity of at least about 70%. In some embodiments, the method further comprises determining a likelihood of the identification of the disease or disorder of the subject. In some embodiments, the method further comprises providing a therapeutic intervention for the disease or disorder of the subject. In some embodiments, the method further comprises monitoring the disease or disorder of the subject, wherein the monitoring comprises assessing the disease or disorder of the subject at a plurality of time points, wherein the assessing is based at least on the disease or disorder identified at each of the plurality of time points.

In some embodiments, selecting the one or more data analysis tools comprises receiving a user selection of the one or more data analysis tools. In some embodiments, selecting the one or more data analysis tools is automatically performed by the computer without receiving a user selection of the one or more data analysis tools.

In another aspect, the present disclosure provides a computer system for assessing a condition of a subject, comprising: a database that is configured to store a dataset of a biological sample of the subject; and one or more computer processors operatively coupled to the database, wherein the one or more computer processors are individually or collectively programmed to: (i) select one or more data analysis tools comprising: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, a Target Scoring analysis tool, or a combination thereof; (ii) process the dataset using the one or more data analysis tools to generate a data signature of the biological sample of the subject; and (iii) based at least in part on the data signature generated in (ii), assess the condition of the subject.

In another aspect, the present disclosure provides a non-transitory computer readable medium comprising machine-executable code that, upon execution by one or more computer processors, implements a method for assessing a condition of a subject, the method comprising: (a) receiving a dataset of a biological sample of the subject; (b) selecting one or more data analysis tools, wherein the one or more data analysis tools comprise an analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool; (c) processing the dataset using the one or more data analysis tools to generate a data signature of the biological sample of the subject; and (d) based at least in part on the data signature generated in (c), assessing the condition of the subject. In any embodiment described herein, the one or more data analysis tools may be a plurality of data analysis tools each independently selected from a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool.

To obtain a blood sample, various techniques may be used, e.g., a syringe or other vacuum suction device. A blood sample may be optionally pre-treated or processed prior to use. A sample, such as a blood sample, may be analyzed under any of the methods and systems herein within 4 weeks, 2 weeks, 1 week, 6 days, 5 days, 4 days, 3 days, 2 days, 1 day, 12 hr, 6 hr, 3 hr, 2 hr, or 1 hr from the time the sample is obtained, or longer if frozen. When obtaining a sample from a subject (e.g., blood sample), the amount may vary depending upon subject size and the condition being screened. In some embodiments, at least 10 mL, 5 mL, 1 mL, 0.5 mL, 250, 200, 150, 100, 50, 40, 30, 20, 10, 9, 8, 7, 6, 5, 4, 3, 2, or 1 μL of a sample is obtained. In some embodiments, 1-50, 2-40, 3-30, or 4-20 μL of sample is obtained. In some embodiments, more than 5, 10, 15, 20, 25, 30, 35, 40, 45, 50, 55, 60, 65, 70, 75, 80, 85, 90, 95 or 100 μL of a sample is obtained.

The sample may be taken before and/or after treatment of a subject with a disease or disorder. Samples may be obtained from a subject during a treatment or a treatment regime. Multiple samples may be obtained from a subject to monitor the effects of the treatment over time. The sample may be taken from a subject known or suspected of having a disease or disorder for which a definitive positive or negative diagnosis is not available via clinical tests. The sample may be taken from a subject suspected of having a disease or disorder. The sample may be taken from a subject experiencing unexplained symptoms, such as fatigue, nausea, weight loss, aches and pains, weakness, or bleeding. The sample may be taken from a subject having explained symptoms. The sample may be taken from a subject at risk of developing a disease or disorder due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

In some embodiments, a sample may be taken at a first time point and assayed, and then another sample may be taken at a subsequent time point and assayed. Such methods may be used, for example, for longitudinal monitoring purposes to track the development or progression of a disease. In some embodiments, the progression of a disease may be tracked before treatment, after treatment, or during the course of treatment, to determine the treatment's effectiveness. For example, a method as described herein may be performed on a subject prior to, and after, treatment with a lupus, psoriasis, atopic dermatitis, and/or systemic sclerosis (scleroderma) condition therapy to measure the disease's progression or regression in response to the lupus, psoriasis, atopic dermatitis, and/or systemic sclerosis (scleroderma) condition therapy.

After obtaining a sample from the subject, the sample may be processed to generate datasets indicative of a disease or disorder of the subject. For example, a presence, absence, or quantitative assessment of nucleic acid molecules of the sample from a panel of condition-associated genomic loci or may be indicative of a lupus, psoriasis, atopic dermatitis, and/or systemic sclerosis (scleroderma) condition of the subject. Processing the sample obtained from the subject may comprise (i) subjecting the sample to conditions that are sufficient to isolate, enrich, or extract a plurality of nucleic acid molecules, and (ii) assaying the plurality of nucleic acid molecules to generate the dataset (e.g., microarray data, nucleic acid sequences, or quantitative polymerase chain reaction (qPCR) data). Methods of assaying may include any assay known in the art or described in the literature, for example, a microarray assay, a sequencing assay (e.g., DNA sequencing, RNA sequencing, or RNA-Seq), or a quantitative polymerase chain reaction (qPCR) assay.

In some embodiments, a plurality of nucleic acid molecules is extracted from the sample and subjected to sequencing to generate a plurality of sequencing reads. The nucleic acid molecules may comprise ribonucleic acid (RNA) or deoxyribonucleic acid (DNA). The extraction method may extract all RNA or DNA molecules from a sample. Alternatively, the extraction method may selectively extract a portion of RNA or DNA molecules from a sample. Extracted RNA molecules from a sample may be converted to cDNA molecules by reverse transcription (RT).

The sample may be processed without any nucleic acid extraction. For example, the disease or disorder may be identified or monitored in the subject by using probes configured to selectively enrich nucleic acid (e.g., RNA or DNA) molecules corresponding to a panel of condition-associated genomic loci. The probes may be nucleic acid primers. The probes may have sequence complementarity with nucleic acid sequences from one or more of the panel of condition-associated genomic loci. The panel of condition-associated genomic loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more condition-associated genomic loci.

The probes may be nucleic acid molecules (e.g., RNA or DNA) having sequence complementarity with nucleic acid sequences (e.g., RNA or DNA) of one or more genomic loci (e.g., condition-associated genomic loci). These nucleic acid molecules may be primers or enrichment sequences. The assaying of the sample using probes that are selective for the one or more genomic loci (e.g., condition-associated genomic loci) may comprise use of array hybridization, polymerase chain reaction (PCR), or nucleic acid sequencing (e.g., RNA sequencing or DNA sequencing, such as RNA-Seq).

The assay readouts may be quantified at one or more genomic loci (e.g., condition-associated genomic loci) to generate the data indicative of the disease or disorder. For example, quantification of array hybridization or polymerase chain reaction (PCR) corresponding to a plurality of genomic loci (e.g., condition-associated genomic loci) may generate data indicative of the disease or disorder. Assay readouts may comprise quantitative PCR (qPCR) values, digital PCR (dPCR) values, digital droplet PCR (ddPCR) values, fluorescence values, etc., or normalized values thereof.

### Big Data Analysis Tools and Drug/Target Scoring Algorithms

The present disclosure provides systems and methods to perform data analysis using drug or target scoring algorithms and/or big data analysis tools. In various aspects, such drug or target scoring algorithms and/or big data analysis tools may be used to perform analysis of data sets including, for example, mRNA gene expression or transcriptome data, DNA genomic data, proteomic data, metabolomic data, other types of “-omic” data, or a combination thereof. Systems and methods of the present disclosure may use one or more of the following: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool.

A non-limiting example of a workflow of a method to assess a condition of a subject using one or more data analysis tools and/or algorithms may comprise receiving a dataset of a biological sample of a subject. Next, the method may comprise selecting one or more data analysis tools and/or algorithms. For example, the data analysis tools and/or algorithms may comprise a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, a Target Scoring analysis tool, or a combination thereof. Next, the method may comprise processing the dataset using selected data analysis tools and/or algorithms to generate a data signature of the biological sample of the subject. Next, the method may comprise assessing the condition of the subject based on the data signature.

The BIG-C (Biologically Informed Gene Clustering) tool may be configured to sort large groups of genes into a set of functional groups (e.g., 53 functional groups). The functional groups are created utilizing publicly available information from online tools and databases including UniProtKB/Swiss-Prot, GO Terms, KEGG pathways, NCBI PubMed, and the Interactome. The functional groups may include one or more of. Active RNA, Anti-apoptosis, anti-proliferation, autophagy, chromatin remodeling, cytoplasm and biochemistry, cytoskeleton, DNA repair, endocytosis, endoplasmic reticulum, endosome and vesicles, fatty acid biosynthesis, cell surface, transcription, glycolysis and gluconeogenesis, golgi, immune cell surface, immune secreted, immune signaling, integrin pathway, interferon stimulated genes, intracellular signaling, lysosome, melanosome, MHC class I, MHC class II, microRNA processing, microRNA, mitochondrial transcription, mitochondria, mitochondria oxidative phosphorylation, mitochondrial TCA cycle, mRNA processing, mRNA splicing, non-coding RNA, nuclear receptor, nucleus and nucleolus, palmitoylation, pattern recognition receptors, peroxisomes, pro-apoptosis, pro-cell cycle, proteasome, pseudogenes, RAS superfamily, reactive oxygen species protection, secreted and extracellular matrix, transcription factors, transporters, transposon control, ubiquitylation and sumoylation, unfolded protein and stress, and unknown. Enrichment scores for each group are calculated based on an overlap p value to determine the functional groups over or under-expressed in the gene expression dataset. The BIG-C may be configured such that each gene is sorted into only one of the 53 functional groups, allowing for a quick and relatively simple understanding of types of genes enriched and co-expressed in a big dataset.

The I-Scope™ tool may be configured to identify immune infiltrates. Hematopoietic cells are unique in that they move throughout the body patrolling for threats to the host, and may infiltrate tissue sites not normally home to immune cells. I-Scope™ may be configured to identify hematopoietic cells through an iterative search of more than 17,000 genes identified in more than 50 microarray datasets. From this search, 1226 candidate genes are identified and researched for restriction in hematopoietic cells as determined by the HPA, GTEx and FANTOM5 datasets (e.g., available at proteinatlas.org). 926 genes meet the criteria for being mainly restricted to hematopoietic lineages (brain, reproductive organ exclusions were permitted). These genes are researched for immune cell specific expression in 27 hematopoietic sub-categories: alpha beta T cell, T cell, regulatory T Cell, activated T cell, anergic T cell, gamma delta T cells, CD8 T, NK/NKT cell, NK cell, T & B cells, B cells, germinal center B cells, B cell and plasmacytoid dendritic cell, T &B & myeloid, B & myeloid, T & myeloid, MHC Class II expressing cell, monocyte, dendritic cell, plasmacytoid dendritic cells, myeloid cell, plasma cell, erythrocyte, neutrophil, low density granulocyte, granulocyte, and platelet. Transcripts are entered into I-Scope™ and the number of transcripts in each category determined. Odd's ratios are calculated with confidence intervals using the Fisher's exact test in R.

The T-Scope™ tool may be configured to help identify types of non-hematopoietic cells in gene expression datasets. T-Scope™ may be configured by downloading approximately 10,000 tissue enriched and 8,000 cell line enriched genes from the human protein atlas along with their tissue or cell line designation (e.g., available at proteinatlas.org). Genes found in more than four tissues are eliminated. Housekeeping genes described in the gene expression study by She et al. are also removed (e.g., as described by She et al., “Definition, conservation and epigenetics of housekeeping and tissue-enriched genes,” BMC Genomics 2009, 10:269, which is incorporated herein by reference in its entirety). This list is further curated by removing genes differentially expressed in 34 hematopoietic cell gene expression datasets and adding kidney specific genes from datasets downloaded from the GEO repository and processed by Ampel BioSolutions. The resulting categories of genes represent genes enriched in the following 42 tissue/cell specific categories: adrenal gland, breast, cartilage, cerebral cortex, uterine cervix, chondrocyte, colon, duodenum, endometrium, epididymis, esophagus fallopian tube, esophagus, fibroblast, heart muscle, keratinocyte, kidney, liver, lung, melanocyte, ovary pancreas, parathyroid gland, placenta, podocyte, prostrate, rectum, salivary gland, seminal vesicle, skeletal muscle, skin, small intestine, smooth muscle, stomach, synoviocyte, testis, kidney loop of henle, kidney proximal tubule, kidney distal tubule, and kidney collecting duct.

The CellScan tool may be a combination of I-Scope™ and T-Scope™, and may be configured to analyse tissues with suspected immune infiltrations that may also have tissue specific genes. CellScan may potentially be more stringent than either I-Scope™ or T-Scope™ because it may be used to distinguish resident tissue cells from non-resident hematopoietic cells.

The MS (Molecular Signature) Scoring tool may be configured to assess specific pathways in a disease state. Information on genes that encode for proteins that participate in a specific signaling pathway, and whether the gene product promotes or inhibits the pathway, are compiled and curated through literature mining. Curated pathways presented by the company include CD40-CD40ligand, IL-6, IL-12/23, TNF, IL-17, IL-21, S1P1, IL-13 and PDE4, but this method may be used for any known signaling pathway with available data. To determine if a signaling pathway is over or under-expressed in a microarray dataset, the gene list for each signaling pathway may be queried against the limma differentially expressed genes from a disease state compared to healthy controls, and the differentially expressed genes in the signaling pathway may be identified for each set. The fold changes for genes that promoted the pathway may be added together and the fold changes for genes that inhibited the pathway may be subtracted from the score. This total score may be normalized based on the number of genes that may be detected on the specific microarray platform used for the experiment. Activation scores of −100 to +100 may be determined using this method with negative scores indicating an inhibition of the specific pathway in the disease state and positive scores indicating an up-regulation of a specific pathway in the disease state. The Fischer's exact test may be performed to determine if there was sufficient overlap of genes between the experimental differentially expressed genes and the genes in the signaling pathway.

Gene Set Variation Analysis (GSVA) may be performed (for example, as described in Catalina et al. (2019, Communications Biology, “Gene expression analysis delineates the potential roles of multiple interferons in systemic lupus erythematosus”, which is incorporated herein by reference in its entirety) to determine enrichment of signaling pathways in individual patient samples. Gene set variation analysis may be performed using an open source software package for the coding language R available at the R Bioconductor (bioconductor.org), e.g., as described by Hanzelman et al., (“GSVA: gene set variation analysis for microarray and RNA-Seq data,” BMC Bioinformatics, 2013, which is incorporated herein by reference in its entirety). The modules of genes to interrogate the datasets may be developed. Modules of genes determined to represent a specific signaling pathway or process may be identified (e.g., using publicly available datasets). For example, the IFNB1 signaling pathway is taken from a publicly available gene expression dataset of peripheral blood cells treated with IFNB1 in vitro. Genes co-expressed in this dataset (genes either all increased or decreased compared to control treated peripheral blood) are used to create modules of genes representing the IFNB1 signaling pathway, and GSVA is used to determine the enrichment of this set of genes and hence the IFNB1 signaling pathway in individual patient and control samples.

The CoLTs®, or Combined Lupus Treatment Scoring, may be configured to rank identified drugs or therapies by a number of essential characteristics, including scientific rationale, experience in lupus mice/human cells (preclinical), previous clinical experience in autoimmunity, drug properties, and safety profile, including adverse events. Face and test validities may be established by scoring SOC medications and confirming the scores with a panel of lupus clinicians. The final result may be the CoLTs® score. A CoLTs® algorithm may also be configured for drugs in development (DID), which typically do not have drug metabolism and adverse event information available.

The target scoring algorithm may be configured to prioritize a specific gene or protein that is potentially a good choice to target with a drug in lupus, psoriasis, atopic dermatitis, and/or systemic sclerosis (scleroderma) patients. It may be utilized even if there is currently no drug available to the target gene or protein. The algorithm may be based on the addition of 18 data based determinations plus the overall scientific rationale and generates scores from −13 (not a good target in SLE) to 27 (very promising target in SLE).

### BIG-C™ Big Data Analysis Tool

BIG-C® is a fast and efficient cloud-based tool to functionally categorize gene products. With coverage of over 80% of the genome, BIG-C® leverages publicly available databases such as UniProtKB/Swiss-Prot, GO terms, KEGG pathways, NCBI PubMed and Interactome to place genes into 53 functional categories. The sorting into only one of 53 functional groups allows for a quick and relatively simple understanding of types of genes enriched and co-expressed in a big dataset. This assists in deriving further insights from genes expressed for a given disease state in human or pre-clinical mouse models.

BIG-C® may be used to functionally categorize immunological genes that are not covered in cancer databases such as GO and KEGG (e.g., as described by Grammer et al. 2016, “Drug repositioning in SLE: crowd-sourcing, literature-mining and Big Data analysis,” Lupus, 25(10), 1150-1170, which is incorporated herein by reference in its entirety). Using a knowledge base of over 5000 patients with systemic lupus erythematosus (SLE), over 16432 genes are each placed into one of 53 BIG-C® functional categories, and statistical analysis is performed to identify enriched categories. BIG-C® categories are cross-examined with the GO and KEGG terms to obtain additional information and insights.

A sample BIG-C® workflow may comprise the following steps. First, SLE genomic datasets are derived from whole blood, peripheral blood mononuclear cells, affected tissues, and purified immune cells. Second, datasets are analyzed using DE analysis (as shown by a differential expression heatmap) or Weighted Gene Coexpression Network Analysis (WGCNA) (as shown by a gene coexpression plot). Third, expressed genes are annotated using publicly available databases (e.g., UniProtKB/Swiss-Prot database, Human Immunodeficiencies database, Mouse MGI database, Entrez Molecular Sequence database, PubMed, and the Human Tissue Atlas). Fourth, signatures are cross-referenced with purified single-cell microarray datasets and RNAseq experiments. Fifth, BIG-C® is leveraged to separate the individual annotated genes into one of 53 functional categories (e.g., as described by Labonte et al. 2018, “Identification of alterations in macrophage activation associated with disease activity in systemic lupus erythematosus,” PloS one, 13(12), e0208132, which is incorporated herein by reference in its entirety). Sixth, chi-squared analysis is used to determine enriched categories of interest from overlap p-values. Seventh, enriched categories are cross-examined with GO and KEGG terms to derive key insights for further analysis.

### I-Scope™ Big Data Analysis Tool

I-Scope™ may be a tool configured for cross-examining the presence and activity of varying types of immune cell infiltrates with observed gene expression patterns. It may take annotated gene expression data and analyze it for hematopoietic cell lineage. I-Scope™ may be used downstream of the BIG-C® (Biologically Informed Gene-Clustering) tool in that it helps to provide even more insight into the nature of the genes being expressed after categorization.

I-Scope™ addresses the need to understand the involvement of specific cells for a given disease state. While it is helpful to understand the relative up-regulation and down-regulation at the gene expression level, it is even more informative to understand specifically in which cells this is occurring. I-Scope™ may be configured to identify hematopoietic cells through an iterative search of more than 17,000 genes identified in more than 50 microarray datasets (e.g., as described by Hubbard et al., “Analysis of Lupus Synovitis Gene Expression Reveals Dysregulation of Pathogenic Pathways Activated within Infiltrating Immune Cells,” Arthritis Rheumatol, 2018; 70 (suppl 10), which is incorporated herein by reference in its entirety). I-Scope™ may function by restricting the analysis to genes of hematopoietic cell heritage and allow for cross-checking against purified single-cell experiments or datasets. The cross-check confirms and categorizes specific transcript signatures to the 28 hematopoietic cell sub-categories, ultimately allowing for cellular activity analysis across multiple samples and disease states. When combined with BIG-C® categories, the cellular activity may be correlated to specific functions within a given cell type.

A sample I-Scope™ workflow may comprise the following steps. First, candidate genes are identified from SLE (systemic lupus erythematosus) datasets potentially associated with immune cell expression. Second, using HPA, GTEx, and FANTOM5 datasets, expression signatures associated with hematopoietic cell lineage are identified. Third, signatures are cross-referenced with purified single-cell microarray datasets and RNAseq experiments. Fourth, transcripts are categorized into 28 hematopoietic cell sub-categories and assess cellular expression across different samples and disease states. Odd's ratios are calculated with confidence intervals using the Fisher's exact test in R. An I-Scope™ signature analysis for a given sample may lead to the I-Scope™ signature analysis across multiple samples and disease states.

### T-Scope™ Big Data Analysis Tool

The T-Scope™ tool may be configured for cross-examining gene expression signatures of a given sample with a database of non-hematopoietic cell types (e.g., as described by Hubbard et al., “Analysis of Gene Expression from Systemic Lupus Erythematosus Synovium Reveals Unique Pathogenic Mechanisms [Abstract], Annual Meeting of the American College of Rheumatology; June 2019; Chicago, IL, which is incorporated herein by reference in its entirety). T-Scope™ may comprise a database of 704 transcripts allocated to 45 independent categories. Transcripts detected in the sample are matched to one of the cellular categories within the T-Scope™ tool to derive further insights on tissue cell activity. T-Scope™ may be used downstream of the BIG-C® (Biologically Informed Gene-Clustering) tool to understand which tissue cell types are present. In conjunction with I-Scope™ (which provides information related to immune cells), T-Scope™ may be performed to provide a complete view of all possible cell activity in a given sample.

T-Scope™ addresses the need to understand the involvement of specific tissue cells for a given disease state. While it is helpful to understand the relative up-regulation and down-regulation at the gene expression level, it is even more informative to understand specifically in which cells this is occurring. T-Scope™ may be configured by downloading a set of approximately 10,000 tissue enriched and 8,000 cell line enriched genes from the Human Protein Atlas along with their tissue or cell line designation. Genes differentially expressed in hematopoietic cell datasets are removed and kidney specific genes are added from the GEO repository. T-Scope™ may function by restricting the analysis to genes of known tissue cell heritage and allow for cross-checking against purified single-cell experiments or datasets. The cross-check confirms and categorizes specific transcript signatures to the 45 tissue cell sub-categories, ultimately allowing for cellular activity analysis across multiple samples and disease states. When combined with BIG-C® categories, the cellular activity may be correlated to specific functions within a given tissue cell type.

A sample T-Scope™ workflow may comprise the following steps. First, candidate genes are identified from SLE (systemic lupus erythematosus) differential expression datasets potentially associated with tissue cell expression. Second, using publicly available databases, expression signatures associated with potential tissue cell activity are identified. Third, signatures are cross-referenced with microarray, scRNAseq or RNAseq experiments. Fourth, transcripts are categorized into 45 tissue cell sub-categories and cellular expression is assessed across different samples and disease states. Results may be obtained using T-Scope™ in combination with I-Scope™ for identification of cells post-DE-analysis.

### CellScan Big Data Analysis Tool

A cloud-based genomic platform may be configured to provide users with access to CellScan™, which comprises a suite of tools for the identification, analysis, and prioritization of targets for drug development and/or repositioning. This platform is powered by a database containing the genomic information gathered from 5000+ autoimmune patients. The cloud-based genomic platform may leverage results from RNAseq and microarray experiments in conjunction with clinical information, such as medication and lab tests, to provide undiscovered insights.

CellScan™ may go beyond typical ‘omics analysis by performing one or more of the following: functionally categorizing genes and their products (e.g., using BIG-C®); deconvolving gene expression data to identify unique immunological cell types from blood or biopsy samples (e.g., using I-Scope™); identifying tissue specific cell from biopsy samples (e.g., using T-Scope™); identifying receptor-ligand interactions and subsequent signaling pathways (e.g., using MS-Scoring™); ranking genes and their products for targeting by drugs and miRNA mimetics (e.g., using Target-Scoring™); and prioritizing FDA-approved drugs and drugs-in-development for treatment in patients or pre-clinical models (e.g., using CoLTs®).

CellScan™ applications may include one or more of: Biomarker Discovery, Disease Mechanisms, Drug Mechanism of Action, Drug Mechanism of Toxicity, and Target Identification and Validation. Experimental approaches supported by CellScan™ may include one or more of lncRNA, Metabolomics, MicroArray, miRNA, mRNA, qPCR, Proteomics, and RNAseq.

Data analysis and interpretation with CellScan™ may build on comprehensive, manually curated content of a knowledge base. Powerful, quick, and efficient tools may be used to perform deep analysis of NGS and miRNA data to identify gene function, immunological and tissue cell type, pathways, and target/drug appropriate for a specific disease state.

CellScan™ features may be configured to optimize or maximize the impact of information that surfaces in an analysis so that interpretation of a dataset is comprehensive and elucidates actionable insights. These features may include one or more of: NGS RNAseq data analysis, biomarker scoring, and prioritizing targets and drugs for human clinical trials and/or pre-clinical models. The NGS RNAseq data analysis may comprise interrogating RNA and miRNA data for function, cell-type (immunological or tissue) and pathways. The biomarker scoring may comprise using a knowledge base and gene expression data to assess and prioritize biomarkers associated with a target disease or phenotype. The target/drug prioritization may comprise leveraging objective scoring of targets and drugs based on parameters such as scientific rationale, evidence in mouse/human cells, prior clinical data, overall drug properties, and the risk of adverse events.

The knowledge base may be a repository created from millions of individual pieces of information gathered about genes, cells, tissues, drugs, and diseases, and manually reviewed for accuracy and includes rich contextual details and links to original publications. The knowledge base may enable access to relevant and substantiated knowledge from primary literature as well as public and private databases for comprehensive interpretation of NGS/RNAseq data elucidating function/pathways and prioritize targets/drugs for given disease states. An example list of reference databases for the content in CellScan™, with both human and mouse species-specific identifiers supported.

### MS (Molecular Signature) Scoring™ Analysis Tool

MS-Scoring™ may be configured to identify receptor-ligand interactions and predict ongoing signaling pathways. In addition, MS-Scoring™ may be used to validate molecular pathways as potential targets for new or repurposed drug therapies. The specificity of next-generation drug therapies requires a way to understand the potential of a given therapy to act on the intended biochemical target. Moreover, a potential application of this is the repositioning of drug therapies that may have the correct biochemical targeting to address multiple clinical needs beyond the initial intended therapeutic value.

MS-Scoring™ may be specifically developed to address gaps in the QIAGEN IPA® (Ingenuity Pathway Analysis) tool that does not contain many immunologically relevant pathways. Similar to IPA®, MS-Scoring™ 1 may use log-fold change information to score the target and its signaling pathway to verify the viability of the targets. If the fold-change of the genes of a signaling pathway appears to be upregulated or inhibitors appear to be downregulated, MS-Scoring™ 1 may provide a score of +1. Conversely if the genes of a signaling pathway appear downregulated or the inhibitors upregulated, MS-Scoring™ 1 may provide a score of −1. A score of zero may be provided if no fold-change is observed. The scores may then be summed and normalized across the entire pathway to yield a final % score between −100 (inhibition) and +100 (up-regulation). Higher absolute magnitude scores, scores that are close to −100 or +100, may indicate a high potential for therapeutic targeting. The Fischer's exact test may be performed to determine if there is sufficient overlap of genes between the experimental differentially expressed genes and the genes in the signaling pathway.

A sample MS-Scoring™ 1 workflow may comprise the following steps. First, potential drugs and pathways are identified by LINCS (Library of Integrated Network-Based Cellular Signatures) as candidates for therapeutic intervention. Second, MS-Scoring™ 1 is used to evaluate individual transcript elements of the target pathway. Third, signatures are cross-referenced with purified single-cell microarray datasets and RNAseq experiments. Fourth, scores are compiled and normalized to provide an overall % score for the pathway and higher absolute magnitude scores indicate a higher potential for therapeutic targeting.

MS-Scoring™ 1 may be performed of IL-12 and IL-23 related pathways for targeting using ustekinumab for SLE (systemic lupus erythematosus) drug repositioning (e.g., as described by Grammer et al., 2016, “Drug repositioning in SLE: crowd-sourcing, literature-mining and Big Data analysis,” Lupus, 25(10), 1150-1170, which is incorporated herein by reference in its entirety).

MS-Scoring™ 2 may utilize custom-defined gene modules that represent a signaling pathway or process and is particularly useful for gene expression datasets from microarray or RNAseq. The MS-Scoring™ 2 tool may be configured to take a deeper look at signaling pathways analyzed using the MS-Scoring™ 1. The tool may analyze raw gene expression data and assess enrichment by the Gene Set Variation Analysis (as described herein), which assigns an indexed score to the individual co-expressed pathways between −1 and +1 indicating levels of down-regulation and up-regulation respectively.

A sample MS-Scoring™ 2 workflow may comprise the following steps. First, a signaling pathway of interest is selected from the MS-Scoring™ 2 menu. Second, a raw gene expression data is inputted into the MS-Scoring™ 2 tool. Third, enrichment of signaling pathway(s) is assessed on a patient by patient basis. Fourth, the data may then be used to drive insight for the target signaling pathways in individual patient samples.

Results from GSVA Analysis on SLE (systemic lupus erythematosus) signaling pathways may be, e.g., as described by Hanzelmann et al., “GSVA: Gene Set Variation Analysis for Microarray and RNA-Seq Data,” BMC Bioinformatics, vol. 14, no. 1, 2013, p. 7, which is incorporated herein by reference in its entirety.

### CoLTs® (Combined Lupus Treatment Scoring) Analysis Tool

A scoring method called CoLTs®, or Combined Lupus Treatment Scoring, may be configured to assessing and prioritizing the repositioning potential of drug therapies. CoLTs® may rank identified drugs/therapies by a number of essential characteristics, including scientific rationale, experience in lupus mice/human cells (preclinical), previous clinical experience in autoimmunity, drug properties, and safety profile, including adverse events. Face and test validities may be established by scoring standard of care (SOC) medications and confirming the scores with a panel of lupus clinicians. The final result may be the CoLTs® score. A CoLTs® algorithm may also be configured for drugs in development (DID) since they typically do not have drug metabolism and adverse event information available.

CoLTs® may be configured to perform objective scoring of drug molecules based on a hypothesis-based literature search of publicly available databases. The tool has the ability to rank drug molecules from both FDA-approved and non-approved classes and ranked based upon parameters such as scientific rationale, evidence in mouse/human cells, prior clinical data, overall drug properties, and the risk of adverse events. The parameters are used within five independent drug therapy categories: small molecules, biologics, complementary and alternative therapies, and drugs in development.

CoLTs® may address the need for a systematic and objective way to evaluate the potential of drug therapies to be repositioned for treatment of autoimmune diseases, initially within SLE (systemic lupus erythematosus). The composite score may embody all the accessible information in literature databases, inclusive of efficacy and adverse reactions, to be able to assist in the prioritization of drug development. While the composite score takes into account many aspects of a drug, it may heavily weigh the risk of adverse events and ranges from −16 to +11. CoLT Scoring® may be validated through repeated scoring of 215 potential therapies using a total of over 5000 reference data points as well as by clinicians specializing in the field of rheumatology. Specifically, CoLTs®′ prediction of Stelara/Ustekinumab to be a top priority biologic for lupus drug repositioning is validated by a successful Phase 2 clinical trial (e.g., as described by Vollenhoven et al., “Efficacy and Safety of Ustekinumab, an IL-12 and IL-23 Inhibitor, in Patients with Active Systemic Lupus Erythematosus: Results of a Multicentre, Double-Blind, Phase 2, Randomised, Controlled Study.” The Lancet, vol. 392, no. 10155, 2018, pp. 1330-1339, which is incorporated herein by reference in its entirety). CoLTs® may be calibrated on SoC (Standard of Care) therapies for the individual autoimmune disease being assessed.

Within the ten major categories, rationale ranges from 0 to +3, mouse/human in vitro experience ranges from −1 to +1, clinical properties are on a scale of −3 to +3, the adverse effect of inducing lupus ranges from −1 to 0, metabolic properties range from −2 to 0, and finally adverse events (such as toxicity, infection, carcinogenic, etc.) were given a score of −5 to 0 (e.g., as described by Grammer et al., 2016, “Drug repositioning in SLE: crowd-sourcing, literature-mining and Big Data analysis,” Lupus, 25(10), 1150-1170, which is incorporated herein by reference in its entirety). For example, CoLT Scoring® of SOC Therapies in Lupus (Belimumab, HCQ, and Rituximab) may be performed.

### Target Scoring Analysis Tool

The Target scoring algorithm may be configured to prioritize a specific gene or protein that would potentially be a good choice to target with a drug in lupus patients. It may be utilized even if there is currently no drug available to the target gene or protein. The algorithm may be based on the addition of 18 data based determinations plus the overall scientific rationale and generates scores from −13 (not a good target in SLE) to 27 (very promising target in SLE).

Target-Scoring™ may be configured to assessing and prioritizing the potential of molecular targets for further development of drug therapies. The Target-Scoring™ tool is very similar to CoLTs® except it approaches the need for new SLE therapies from a different angle. Target Scoring may be configured to perform an objective assessment of molecular targets for the development of new or repurposed drug therapies. Like CoLTs®, it also derives data from a hypothesis-based literature search and generates a composite score based on the publicly available information. Leveraging the composite score, researchers may better prioritize the development of novel drug therapies addressing the assessed targets of interest.

Target-Scoring™ may utilize 19 different scoring categories to derive a composite score that ranges from −13 to +27 for the suitability of a gene target for SLE therapy development. Target-Scoring™ may be validated through repeated scoring of potential therapies as well as by clinicians (e.g., clinicians specializing in the field of immunology).

### Classifiers

### Assessment of Conditions

A non-limiting example of a method to assess a condition of a subject, e.g., an SLE, DLE, PSO, AD, or SSc condition, may comprise one or more of the following operations. A dataset of a biological sample of a subject is received. The dataset may comprise quantitative measures of gene expression from each of a plurality of lupus-associated genomic loci.

A patient sample may be harvested using any method known to those of skill in the art. To obtain a blood sample, various techniques may be used, e.g., a syringe or other vacuum suction device. A blood sample may be optionally pre-treated or processed prior to use. A sample, such as a blood sample, may be analyzed under any of the methods and systems herein within 4 weeks, 2 weeks, 1 week, 6 days, 5 days, 4 days, 3 days, 2 days, 1 day, 12 hr, 6 hr, 3 hr, 2 hr, or 1 hr from the time the sample is obtained, or longer if frozen. When obtaining a sample from a subject (e.g., blood sample), the amount may vary depending upon subject size and the condition being screened. In some embodiments, at least 10 mL, 5 mL, 1 mL, 0.5 mL, 250, 200, 150, 100, 50, 40, 30, 20, 10, 9, 8, 7, 6, 5, 4, 3, 2, or 1 μL of a sample is obtained. In some embodiments, 1-50, 2-40, 3-30, or 4-20 μL of sample is obtained. In some embodiments, more than 5, 10, 15, 20, 25, 30, 35, 40, 45, 50, 55, 60, 65, 70, 75, 80, 85, 90, 95 or 100 μL of a sample is obtained.

To obtain a skin biopsy sample, various techniques may be used. The skin biopsy sample can include skin samples removed from the body of the subject. In certain embodiments, the skin biopsy sample include cells and/or tissues from cutaneous, intradermal, or subcutaneous layer, or from any abnormal tissue in theses layers. In certain embodiments, the skin biopsy sample includes cutaneous tissues. In certain embodiments, the skin biopsy sample includes subcutaneous tissues. Skin biopsy can be performed using any suitable technique known to those of skill in the art. In certain embodiments, skin biopsy can be performed using shave biopsy, punch biopsy, excisional biopsy, or any combination thereof. In certain embodiments, the shave biopsy procedure includes removing a small section of the top layers of skin (epidermis and a portion of the dermis). In certain embodiments, the punch biopsy procedure includes using a tool, such as a circular tool to remove a small core of skin, including deeper layers (epidermis, dermis and superficial fat). In certain embodiments, the excisional biopsy procedure includes removing a lump, lesion and/or an area of abnormal skin. In certain particular embodiments, entire or effectively entire lump, lesion and/or the area of abnormal skin is removed. In certain particular embodiments, the lump, lesion and/or area of abnormal skin is removed through fatty layer of the skin. The area, size, and amount of the skin biopsy sample may vary depending upon the condition being analyzed. In some embodiments, the skin sample is obtained via one, two, three, four, five, or more shave biopsies, punch biopsies, incisional or excisional biopsies, or a combination of the above. In some embodiments, the skin sample comprises, for example, elastin and/or collagen. The skin sample may be obtained from any desired anatomical location on the body, including one or more of the scalp, face, neck, chest, arms, legs, hands, back, buttocks, upper or lower extremities, or genitalia for example. The skin sample may have any appropriate depth. In some embodiments the skin sample has a depth of about 2 mm to about 25 mm. In some embodiments the skin sample has a depth of about 2 mm to about 3 mm, about 2 mm to about 4 mm, about 2 mm to about 5 mm, about 2 mm to about 6 mm, about 2 mm to about 7 mm, about 2 mm to about 8 mm, about 2 mm to about 9 mm, about 2 mm to about 10 mm, about 2 mm to about 15 mm, about 2 mm to about 20 mm, about 2 mm to about 25 mm, about 3 mm to about 4 mm, about 3 mm to about 5 mm, about 3 mm to about 6 mm, about 3 mm to about 7 mm, about 3 mm to about 8 mm, about 3 mm to about 9 mm, about 3 mm to about 10 mm, about 3 mm to about 15 mm, about 3 mm to about 20 mm, about 3 mm to about 25 mm, about 4 mm to about 5 mm, about 4 mm to about 6 mm, about 4 mm to about 7 mm, about 4 mm to about 8 mm, about 4 mm to about 9 mm, about 4 mm to about 10 mm, about 4 mm to about 15 mm, about 4 mm to about 20 mm, about 4 mm to about 25 mm, about 5 mm to about 6 mm, about 5 mm to about 7 mm, about 5 mm to about 8 mm, about 5 mm to about 9 mm, about 5 mm to about 10 mm, about 5 mm to about 15 mm, about 5 mm to about 20 mm, about 5 mm to about 25 mm, about 6 mm to about 7 mm, about 6 mm to about 8 mm, about 6 mm to about 9 mm, about 6 mm to about 10 mm, about 6 mm to about 15 mm, about 6 mm to about 20 mm, about 6 mm to about 25 mm, about 7 mm to about 8 mm, about 7 mm to about 9 mm, about 7 mm to about 10 mm, about 7 mm to about 15 mm, about 7 mm to about 20 mm, about 7 mm to about 25 mm, about 8 mm to about 9 mm, about 8 mm to about 10 mm, about 8 mm to about 15 mm, about 8 mm to about 20 mm, about 8 mm to about 25 mm, about 9 mm to about 10 mm, about 9 mm to about 15 mm, about 9 mm to about 20 mm, about 9 mm to about 25 mm, about 10 mm to about 15 mm, about 10 mm to about 20 mm, about 10 mm to about 25 mm, about 15 mm to about 20 mm, about 15 mm to about 25 mm, or about 20 mm to about 25 mm. In some embodiments the skin sample has a depth of about 2 mm, about 3 mm, about 4 mm, about 5 mm, about 6 mm, about 7 mm, about 8 mm, about 9 mm, about 10 mm, about 15 mm, about 20 mm, or about 25 mm. In some embodiments the skin sample has a depth of at least about 2 mm, about 3 mm, about 4 mm, about 5 mm, about 6 mm, about 7 mm, about 8 mm, about 9 mm, about 10 mm, about 15 mm, or about 20 mm. In some embodiments the skin sample has a depth of at most about 3 mm, about 4 mm, about 5 mm, about 6 mm, about 7 mm, about 8 mm, about 9 mm, about 10 mm, about 15 mm, about 20 mm, or about 25 mm. The skin sample can include one or more layers of the epidermis, dermis, and hypodermis. Skin sampling techniques for disease analysis are widely described in the literature, e.g., by the Mayo Clinic on their website (mayoclinic.org), available under “Skin Biopsy” (Mayo Clinic, Rochester, MN), incorporated herein by reference in its entirety.

The sample may be taken before and/or after treatment of a subject with a disease or disorder. Samples may be obtained from a subject during a treatment or a treatment regime. Multiple samples may be obtained from a subject to monitor the effects of the treatment over time. The sample may be taken from a subject known or suspected of having a disease or disorder for which a definitive positive or negative diagnosis is not available via clinical tests. The sample may be taken from a subject suspected of having a disease or disorder. The sample may be taken from a subject experiencing unexplained symptoms, such as fatigue, nausea, weight loss, aches and pains, weakness, or bleeding. The sample may be taken from a subject having explained symptoms. The sample may be taken from a subject at risk of developing a disease or disorder due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

In some embodiments, a sample may be taken at a first time point and assayed, and then another sample may be taken at a subsequent time point and assayed. Such methods may be used, for example, for longitudinal monitoring purposes to track the development or progression of a disease or disorder (e.g., an SLE condition). In some embodiments, the progression of a disease may be tracked before treatment, after treatment, or during the course of treatment, to determine the treatment's effectiveness. For example, a method as described herein may be performed on a subject prior to, and after, treatment with an SLE therapy to measure the disease's progression or regression in response to the SLE therapy.

After obtaining a sample from the subject, the sample may be processed to generate datasets indicative of a condition (e.g., an SLE condition) of the subject. For example, a presence, absence, or quantitative assessment of nucleic acid molecules of the sample from a panel of condition-associated (e.g., SLE-associated) genomic loci or may be indicative of a condition (e.g., an SLE condition) of the subject. Processing the sample obtained from the subject may comprise (i) subjecting the sample to conditions that are sufficient to isolate, enrich, or extract a plurality of nucleic acid molecules, and (ii) assaying the plurality of nucleic acid molecules to generate the dataset (e.g., microarray data, nucleic acid sequences, or quantitative polymerase chain reaction (qPCR) data). Methods of assaying may include any assay known in the art or described in the literature, for example, a microarray assay, a sequencing assay (e.g., DNA sequencing, RNA sequencing, or RNA-Seq), or a quantitative polymerase chain reaction (qPCR) assay.

In some embodiments, a plurality of nucleic acid molecules is extracted from the sample and subjected to sequencing to generate a plurality of sequencing reads. The nucleic acid molecules may comprise ribonucleic acid (RNA) or deoxyribonucleic acid (DNA). The extraction method may extract all RNA or DNA molecules from a sample. Alternatively, the extraction method may selectively extract a portion of RNA or DNA molecules from a sample. Extracted RNA molecules from a sample may be converted to cDNA molecules by reverse transcription (RT).

The sample may be processed without any nucleic acid extraction. For example, the disease or disorder may be identified or monitored in the subject by using probes configured to selectively enrich nucleic acid (e.g., RNA or DNA) molecules corresponding to a panel of SLE-associated genomic loci. The probes may be nucleic acid primers. The probes may have sequence complementarity with nucleic acid sequences from one or more of the panel of condition-associated (e.g., SLE-associated) genomic loci. The panel of condition-associated genomic loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more condition-associated genomic loci.

The probes may be nucleic acid molecules (e.g., RNA or DNA) having sequence complementarity with nucleic acid sequences (e.g., RNA or DNA) of one or more genomic loci (e.g., condition-associated genomic loci). These nucleic acid molecules may be primers or enrichment sequences. The assaying of the sample using probes that are selective for the one or more genomic loci (e.g., condition-associated genomic loci) may comprise use of array hybridization, polymerase chain reaction (PCR), or nucleic acid sequencing (e.g., RNA sequencing or DNA sequencing, such as RNA-Seq).

The assay readouts may be quantified at one or more genomic loci (e.g., condition-associated genomic loci) to generate the data indicative of the disease or disorder. For example, quantification of array hybridization or polymerase chain reaction (PCR) corresponding to a plurality of genomic loci (e.g., condition-associated genomic loci) may generate data indicative of the disease or disorder. Assay readouts may comprise quantitative PCR (qPCR) values, digital PCR (dPCR) values, digital droplet PCR (ddPCR) values, fluorescence values, etc., or normalized values thereof.

### Classifiers

In some embodiments, the present disclosure provides a system, method, or kit having data analysis realized in software application, computing hardware, or both. In various embodiments, the analysis application or system includes at least a data receiving module, a data pre-processing module, a data analysis module, a data interpretation module, or a data visualization module. In one embodiments, the data receiving module may comprise computer systems that connect laboratory hardware or instrumentation with computer systems that process laboratory data. In one embodiments, the data pre-processing module may comprise hardware systems or computer software that performs operations on the data in preparation for analysis. Examples of operations that may be applied to the data in the pre-processing module include affine transformations, denoising operations, data cleaning, reformatting, or subsampling. A data analysis module, which may be specialized for analyzing genomic data from one or more genomic materials, can, for example, take assembled genomic sequences and perform probabilistic and statistical analysis to identify abnormal patterns related to a disease, pathology, state, risk, condition, or phenotype. A data interpretation module may use analysis methods, for example, drawn from statistics, mathematics, or biology, to support understanding of the relation between the identified abnormal patterns and health conditions, functional states, prognoses, or risks. A data visualization module may use methods of mathematical modeling, computer graphics, or rendering to create visual representations of data that may facilitate the understanding or interpretation of results.

Feature sets may be generated from datasets obtained using one or more assays of a biological sample obtained or derived from a subject, and a trained algorithm may be used to process one or more of the feature sets to identify or assess a condition (e.g., a disease or disorder, such as a lupus, psoriasis, atopic dermatitis, and/or systemic sclerosis (scleroderma) condition) of a subject. For example, the trained algorithm may be used to apply a machine learning classifier to a plurality of condition-associated genomic loci that are associated with two or more classes of individuals inputted into a machine learning model, in order to classify a subject into one of the two or more classes of individuals. For example, the trained algorithm may be used to apply a machine learning classifier to a plurality of condition-associated that are associated with individuals with known conditions (e.g., a disease or disorder, such as a lupus, psoriasis, atopic dermatitis, and/or systemic sclerosis (scleroderma) condition) and individuals not having the condition (e.g., healthy individuals, or individuals who do not have a lupus, psoriasis, atopic dermatitis, and/or systemic sclerosis (scleroderma) condition), in order to classify a subject as having the condition (e.g., positive test outcome) or not having the condition (e.g., negative test outcome).

The trained algorithm may be configured to identify the presence (e.g., positive test result) or absence (e.g., negative test result) of one or more conditions (e.g., a disease or disorder, such as a lupus, psoriasis, atopic dermatitis, and/or systemic sclerosis (scleroderma) condition) with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99%. This accuracy may be achieved for a set of at least about 25, at least about 50, at least about 100, at least about 150, at least about 200, at least about 250, at least about 300, at least about 350, at least about 400, at least about 450, at least about 500, at least about 1,000, or more than about 1,000 independent samples.

The trained algorithm may comprise a machine learning algorithm, such as a supervised machine learning algorithm. The supervised machine learning algorithm may comprise, for example, a Random Forest, a support vector machine (SVM), a neural network, or a deep learning algorithm. The trained algorithm may comprise a classification and regression tree (CART) algorithm. The trained algorithm may comprise an unsupervised machine learning algorithm.

The trained algorithm may comprise a classifier configured to accept as input a plurality of input variables or features (e.g., condition-associated genomic loci) and to produce or output one or more output values based on the plurality of input variables or features (e.g., condition-associated genomic loci). The plurality of input variables or features may comprise one or more datasets indicative of the presence (e.g., positive test result) or absence (e.g., negative test result) of one or more conditions (e.g., a disease or disorder, such as a lupus, psoriasis, atopic dermatitis, and/or systemic sclerosis (scleroderma) condition). For example, an input variable or feature may comprise a number of sequences corresponding to or aligning to each of the plurality of condition-associated genomic loci.

The plurality of input variables or features may also include clinical information of a subject, such as health data. For example, the health data of a subject may comprise one or more of: a diagnosis of one or more conditions (e.g., a disease or disorder, such as a lupus, psoriasis, atopic dermatitis, and/or systemic sclerosis (scleroderma) condition), a prognosis of one or more conditions (e.g., a disease or disorder, such as a lupus, psoriasis, atopic dermatitis, and/or systemic sclerosis (scleroderma) condition), a risk of having one or more conditions (e.g., a disease or disorder, such as a lupus, psoriasis, atopic dermatitis, and/or systemic sclerosis (scleroderma) condition), a treatment history of one or more conditions (e.g., a disease or disorder, such as a lupus, psoriasis, atopic dermatitis, and/or systemic sclerosis (scleroderma) condition), a history of previous treatment for one or more conditions (e.g., a disease or disorder, such as a lupus, psoriasis, atopic dermatitis, and/or systemic sclerosis (scleroderma) condition), a history of prescribed medications, a history of prescribed medical devices, age, height, weight, sex, smoking status, and one or more symptoms of the subject.

For example, the disease or disorder may comprise one or more of: systemic lupus erythematosus (SLE), discoid lupus erythematosus (DLE), lupus nephritis (LN), psoriasis (PSO), atopic dermatitis (AD), or systemic sclerosis (scleroderma, SSc). As another example, the symptoms may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof. As another example, the prescribed medications or drugs may include one or more of: antimalarials, corticosteroids, immunosuppressants, and nonsteroidal anti-inflammatory drugs (NSAIDs).

The trained algorithm may comprise a classifier, such that each of the one or more output values comprises one of a fixed number of possible values (e.g., a linear classifier, a logistic regression classifier, etc.) indicating a classification of the sample by the classifier. The trained algorithm may comprise a binary classifier, such that each of the one or more output values comprises one of two values (e.g., {0, 1}, {positive, negative}, or {high-risk, low-risk}) indicating a classification of the sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the sample by the classifier.

The classifier may be configured to classify samples by assigning output values, which may comprise descriptive labels, numerical values, or a combination thereof. Some of the output values may comprise descriptive labels. Such descriptive labels may provide an identification or indication of the presence (e.g., positive test result) or absence (e.g., negative test result) of one or more conditions (e.g., a disease or disorder, such as a lupus, psoriasis, atopic dermatitis, and/or systemic sclerosis (scleroderma) condition) of the subject, and may comprise, for example, positive, negative, high-risk, intermediate-risk, low-risk, or indeterminate. Such descriptive labels may provide an identification of a treatment for the one or more conditions of the subject, and may comprise, for example, a therapeutic intervention, a duration of the therapeutic intervention, and/or a dosage of the therapeutic intervention suitable to treat the one or more conditions of the subject. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof. For example, such descriptive labels may provide a prognosis of the one or more conditions of the subject. As another example, such descriptive labels may provide a relative assessment of the one or more conditions of the subject. Some descriptive labels may be mapped to numerical values, for example, by mapping “positive” to 1 and “negative” to 0.

The classifier may be configured to classify samples by assigning output values that comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the one or more conditions (e.g., a disease or disorder, such as a lupus, psoriasis, atopic dermatitis, and/or systemic sclerosis (scleroderma) condition) of the subject. Some numerical values may be mapped to descriptive labels, for example, by mapping 1 to “positive” and 0 to “negative.”

The classifier may be configured to classify samples by assigning output values based on one or more cutoff values. For example, a binary classification of samples may assign an output value of “positive” or 1 if the sample indicates that the subject has at least a 50% probability of having one or more conditions (e.g., a disease or disorder, such as a lupus, psoriasis, atopic dermatitis, and/or systemic sclerosis (scleroderma) condition), thereby assigning the subject to a class of individuals receiving a positive test result. As another example, a binary classification of samples may assign an output value of “negative” or 0 if the sample indicates that the subject has less than a 50% probability of having one or more conditions (e.g., a disease or disorder), thereby assigning the subject to a class of individuals receiving a negative test result. In this case, a single cutoff value of 50% is used to classify samples into one of the two possible binary output values or classes of individuals (e.g., those receiving a positive test result and those receiving a negative test result). Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, the classifier may be configured to classify samples by assigning an output value of “positive” or 1 if the sample indicates that the subject has a probability of having one or more conditions (e.g., a disease or disorder, such as a lupus, psoriasis, atopic dermatitis, and/or systemic sclerosis (scleroderma) condition) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having one or more conditions (e.g., a disease or disorder, such as a lupus, psoriasis, atopic dermatitis, and/or systemic sclerosis (scleroderma) condition) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classifier may be configured to classify samples by assigning an output value of “negative” or 0 if the sample indicates that the subject has a probability of having one or more conditions (e.g., a disease or disorder, such as a lupus, psoriasis, atopic dermatitis, and/or systemic sclerosis (scleroderma) condition) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having one or more conditions (e.g., a disease or disorder, such as a lupus, psoriasis, atopic dermatitis, and/or systemic sclerosis (scleroderma) condition) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classifier may be configured to classify samples by assigning an output value of “indeterminate” or 2 if the sample is not classified as “positive”, “negative”, 1, or 0. In this case, a set of two cutoff values is used to classify samples into one of the three possible output values or classes of individuals (e.g., corresponding to outcome groups of individuals having “low risk,” “intermediate risk,” and “high risk” of having one or more conditions, such as a disease or disorder). Examples of sets of cutoff values may include {1%, 99%}{2%, 98%}, {5%, 95%}{110%, 90%}{15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values or classes of individuals, where n is any positive integer.

The trained algorithm may be trained with a plurality of independent training samples. Each of the independent training samples may comprise a sample from a subject, associated datasets obtained by assaying the sample (as described elsewhere herein), and one or more known output values or classes of individuals corresponding to the sample (e.g., a clinical diagnosis, prognosis, absence, or treatment efficacy of a condition of the subject). Independent training samples may comprise samples and associated datasets and outputs obtained or derived from a plurality of different subjects. Independent training samples may comprise samples and associated datasets and outputs obtained at a plurality of different time points from the same subject (e.g., on a regular basis such as weekly, biweekly, or monthly), as part of a longitudinal monitoring of a subject before, during, and after a course of treatment for one or more conditions of the subject. Independent training samples may be associated with presence of the condition (e.g., training samples comprising samples and associated datasets and outputs obtained or derived from a plurality of subjects known to have the condition). Independent training samples may be associated with absence of the condition (e.g., training samples comprising samples and associated datasets and outputs obtained or derived from a plurality of subjects who are known to not have a previous diagnosis of the condition or who have received a negative test result for the condition).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise samples associated with presence of the condition and/or samples associated with absence of the condition. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the condition (e.g., a disease or disorder, such as a lupus, psoriasis, atopic dermatitis, and/or systemic sclerosis (scleroderma) condition). The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with absence of the condition (e.g., a disease or disorder, such as a lupus, psoriasis, atopic dermatitis, and/or systemic sclerosis (scleroderma) condition). In some embodiments, the sample is independent of samples used to train the trained algorithm.

The trained algorithm may be trained with a first number of independent training samples associated with a presence of the condition (e.g., a disease or disorder, such as a lupus, psoriasis, atopic dermatitis, and/or systemic sclerosis (scleroderma) condition) and a second number of independent training samples associated with an absence of the condition (e.g., a disease or disorder, such as a lupus, psoriasis, atopic dermatitis, and/or systemic sclerosis (scleroderma) condition). The first number of independent training samples associated with presence of the condition (e.g., a disease or disorder, such as a lupus, psoriasis, atopic dermatitis, and/or systemic sclerosis (scleroderma) condition) may be no more than the second number of independent training samples associated with absence of the condition (e.g., a disease or disorder, such as a lupus, psoriasis, atopic dermatitis, and/or systemic sclerosis (scleroderma) condition). The first number of independent training samples associated with a presence of the condition (e.g., a disease or disorder) may be equal to the second number of independent training samples associated with an absence of the condition (e.g., a disease or disorder, such as a lupus, psoriasis, atopic dermatitis, and/or systemic sclerosis (scleroderma) condition). The first number of independent training samples associated with a presence of the condition (e.g., a disease or disorder, such as a lupus, psoriasis, atopic dermatitis, and/or systemic sclerosis (scleroderma) condition) may be greater than the second number of independent training samples associated with an absence of the condition (e.g., a disease or disorder, such as a lupus, psoriasis, atopic dermatitis, and/or systemic sclerosis (scleroderma) condition).

The trained algorithm may comprise a classifier configured to identify the presence (e.g., positive test result) or absence (e.g., negative test result) of one or more conditions (e.g., a disease or disorder, such as a lupus, psoriasis, atopic dermatitis, and/or systemic sclerosis (scleroderma) condition) at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the presence (e.g., positive test result) or absence (e.g., negative test result) of the one or more conditions by the trained algorithm may be calculated as the percentage of independent test samples (e.g., subjects known to have the condition or subjects with negative clinical test results for the condition) that are correctly identified or classified as having or not having the condition.

The trained algorithm may comprise a classifier configured to identify one or more conditions (e.g., a disease or disorder, such as a lupus, psoriasis, atopic dermatitis, and/or systemic sclerosis (scleroderma) condition) with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the condition using the trained algorithm may be calculated as the percentage of samples identified or classified as having the condition that correspond to subjects that truly have the condition.

The trained algorithm may comprise a classifier configured to identify one or more conditions (e.g., a disease or disorder, such as a lupus, psoriasis, atopic dermatitis, and/or systemic sclerosis (scleroderma) condition) with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the condition using the trained algorithm may be calculated as the percentage of samples identified or classified as not having the condition that correspond to subjects that truly do not have the condition.

The trained algorithm may comprise a classifier configured to identify one or more conditions (e.g., a disease or disorder, such as a lupus, psoriasis, atopic dermatitis, and/or systemic sclerosis (scleroderma) condition) with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the condition using the trained algorithm may be calculated as the percentage of independent test samples associated with presence of the condition (e.g., subjects known to have the condition) that are correctly identified or classified as having the condition.

The trained algorithm may comprise a classifier configured to identify one or more conditions (e.g., a disease or disorder, such as a lupus, psoriasis, atopic dermatitis, and/or systemic sclerosis (scleroderma) condition) with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the condition using the trained algorithm may be calculated as the percentage of independent test samples associated with absence of the condition (e.g., subjects with negative clinical test results for the condition) that are correctly identified or classified as not having the condition.

The trained algorithm may comprise a classifier configured to identify the presence (e.g., positive test result) or absence (e.g., negative test result) of one or more conditions (e.g., a disease or disorder, such as a lupus, psoriasis, atopic dermatitis, and/or systemic sclerosis (scleroderma) condition) with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying samples as having or not having the condition.

Classifiers of the trained algorithm may be adjusted or tuned to improve or optimize one or more performance metrics, such as accuracy, PPV, NPV, clinical sensitivity, clinical specificity, AUC, or a combination thereof (e.g., a performance index incorporating a plurality of such performance metrics, such as by calculating a weight sum therefrom), of identifying the presence (e.g., positive test result) or absence (e.g., negative test result) of the condition. The classifiers may be adjusted or tuned by adjusting parameters of the classifiers (e.g., a set of cutoff values used to classify a sample as described elsewhere herein, or weights of a neural network) to improve or optimize the performance metrics. The one or more classifiers may be adjusted or tuned so as to reduce an overall classification error (e.g., an “out-of-bag” or oob error rate for a Random Forest classifier). The one or more classifiers may be adjusted or tuned continuously during the training process (e.g., as sample datasets are added to the training set) or after the training process has completed.

The trained algorithm may comprise a plurality of classifiers (e.g., an ensemble) such that the plurality of classifications or outcome values of the plurality of classifiers may be combined to produce a single classification or outcome value for the sample. For example, a sum or a weighted sum of the plurality of classifications or outcome values of the plurality of classifiers may be calculated to produce a single classification or outcome value for the sample. As another example, a majority vote of the plurality of classifications or outcome values of the plurality of classifiers may be identified to produce a single classification or outcome value for the sample. In this manner, a single classification or outcome value may be produced for the sample having greater confidence or statistical significance than the individual classifications or outcome values produced by each of the plurality of classifiers.

After the trained algorithm is initially trained, a subset of the inputs may be identified as most influential or most important to be included for making high-quality classifications (e.g., having highest permutation feature importance). For example, a subset of the panel of condition-associated genomic loci may be identified as most influential or most important to be included for making high-quality classifications or identifications of conditions (or sub-types of conditions). The panel of condition-associated genomic loci, or a subset thereof, may be ranked based on classification metrics indicative of each influence or importance of each individual condition-associated genomic locus toward making high-quality classifications or identifications of conditions (or sub-types of conditions). Such metrics may be used to reduce, in some cases significantly, the number of input variables (e.g., predictor variables) that may be used to train the one or more classifiers of the trained algorithm to a desired performance level (e.g., based on a desired minimum accuracy, PPV, NPV, clinical sensitivity, clinical specificity, AUC, or a combination thereof).

For example, if training a classifier of the trained algorithm with a plurality comprising several dozen or hundreds of input variables to the classifier results in an accuracy of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality may yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

As another example, if training a classifier of the trained algorithm with a plurality comprising several dozen or hundreds of input variables to the classifier results in a sensitivity or specificity of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality may yield decreased but still acceptable sensitivity or specificity of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

The subset of the plurality of input variables (e.g., the panel of condition-associated genomic loci) to the classifier of the trained algorithm may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics (e.g., permutation feature importance).

Upon identifying the subject as having one or more conditions (e.g., a disease or disorder, such as a lupus, psoriasis, atopic dermatitis, and/or systemic sclerosis (scleroderma) condition), the subject may be optionally provided with a therapeutic intervention (e.g., prescribing an appropriate course of treatment to treat the one or more conditions of the subject). The therapeutic intervention may comprise a prescription of an effective dose of a drug, a further testing or evaluation of the condition, a further monitoring of the condition, or a combination thereof. If the subject is currently being treated for the condition with a course of treatment, the therapeutic intervention may comprise a subsequent different course of treatment (e.g., to increase treatment efficacy due to non-efficacy of the current course of treatment).

The therapeutic intervention may include prescribed medications or drugs, which may include one or more of: antimalarials, corticosteroids, immunosuppressants, and nonsteroidal anti-inflammatory drugs (NSAIDs). The therapeutic intervention may be effective to alleviate or decrease one or more symptoms, which may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof.

The therapeutic intervention may comprise recommending the subject for a secondary clinical test to confirm a diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

The feature sets (e.g., comprising quantitative measures of a panel of condition-associated genomic loci) may be analyzed and assessed (e.g., using a trained algorithm comprising one or more classifiers) over a duration of time to monitor a patient (e.g., subject who has a condition or who is being treated for a condition). In such cases, the feature sets of the patient may change during the course of treatment. For example, the quantitative measures of the feature sets of a patient with decreasing risk of the condition due to an effective treatment may shift toward the profile or distribution of a healthy subject (e.g., a subject without the condition). Conversely, for example, the quantitative measures of the feature sets of a patient with increasing risk of the condition due to an ineffective treatment may shift toward the profile or distribution of a subject with higher risk of the condition or a more advanced stage or severity of the condition.

The condition of the subject may be monitored by monitoring a course of treatment for treating the condition of the subject. The monitoring may comprise assessing the condition of the subject at two or more time points. The assessing may be based at least on the feature sets (e.g., quantitative measures of a panel of condition-associated genomic loci) determined at each of the two or more time points. The therapeutic intervention may include prescribed medications or drugs, which may include one or more of: antimalarials, corticosteroids, immunosuppressants, and nonsteroidal anti-inflammatory drugs (NSAIDs). The therapeutic intervention may be effective to alleviate or decrease one or more symptoms, which may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof. The assessing may be based at least on the presence, absence, or severity of one or more symptoms, such as alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof.

In some embodiments, a difference in the feature sets (e.g., quantitative measures of a panel of condition-associated genomic loci) determined between the two or more time points may be indicative of one or more clinical indications, such as (i) a diagnosis of the condition of the subject, (ii) a prognosis of the condition of the subject, (iii) an increased risk of the condition of the subject, (iv) a decreased risk of the condition of the subject, (v) an efficacy of the course of treatment for treating the condition of the subject, and (vi) a non-efficacy of the course of treatment for treating the condition of the subject.

In some embodiments, a difference in the feature sets (e.g., quantitative measures of a panel of condition-associated genomic loci) determined between the two or more time points may be indicative of a diagnosis of the condition of the subject. For example, if the condition was not detected in the subject at an earlier time point but was detected in the subject at a later time point, then the difference is indicative of a diagnosis of the condition of the subject. A clinical action or decision may be made based on this indication of diagnosis of the condition of the subject, such as, for example, prescribing a new therapeutic intervention for the subject. The clinical action or decision may comprise recommending the subject for a secondary clinical test to confirm the diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of a prognosis of the condition of the subject.

In some embodiments, a difference in the feature sets (e.g., quantitative measures of a panel of condition-associated genomic loci) determined between the two or more time points may be indicative of the subject having an increased risk of the condition. For example, if the condition was detected in the subject both at an earlier time point and at a later time point, and if the difference is a negative difference (e.g., the quantitative measures of a panel of condition-associated genomic loci increased from the earlier time point to the later time point), then the difference may be indicative of the subject having an increased risk of the condition. A clinical action or decision may be made based on this indication of the increased risk of the condition, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of the subject having a decreased risk of the condition. For example, if the condition was detected in the subject both at an earlier time point and at a later time point, and if the difference is a positive difference (e.g., the quantitative measures of a panel of condition-associated genomic loci decreased from the earlier time point to the later time point), then the difference may be indicative of the subject having a decreased risk of the condition. A clinical action or decision may be made based on this indication of the decreased risk of the condition (e.g., continuing or ending a current therapeutic intervention) for the subject. The clinical action or decision may comprise recommending the subject for a secondary clinical test to confirm the decreased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of an efficacy of the course of treatment for treating the condition of the subject. For example, if the condition was detected in the subject at an earlier time point but was not detected in the subject at a later time point, then the difference may be indicative of an efficacy of the course of treatment for treating the condition of the subject. A clinical action or decision may be made based on this indication of the efficacy of the course of treatment for treating the condition of the subject, e.g., continuing or ending a current therapeutic intervention for the subject. The clinical action or decision may comprise recommending the subject for a secondary clinical test to confirm the efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of a non-efficacy of the course of treatment for treating the condition of the subject. For example, if the condition was detected in the subject both at an earlier time point and at a later time point, and if the difference is a negative or zero difference (e.g., the quantitative measures of a panel of condition-associated genomic loci increased or remained at a constant level from the earlier time point to the later time point), and if an efficacious treatment was indicated at an earlier time point, then the difference may be indicative of a non-efficacy of the course of treatment for treating the condition of the subject. A clinical action or decision may be made based on this indication of the non-efficacy of the course of treatment for treating the condition of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In various embodiments, machine learning methods are applied to distinguish samples in a population of samples. In one embodiments, machine learning methods are applied to distinguish samples between healthy and diseased (e.g., a lupus condition such as SLE or DLE, psoriasis, atopic dermatitis, or systemic sclerosis (scleroderma)) samples.

### Kits

The present disclosure provides kits for identifying or monitoring a disease or disorder (e.g., a lupus, psoriasis, atopic dermatitis, and/or systemic sclerosis (scleroderma) condition) of a subject. A kit may comprise probes for identifying a quantitative measure (e.g., indicative of a presence, absence, or relative amount) of sequences at each of a panel of condition-associated genomic loci in a sample of the subject. A quantitative measure (e.g., indicative of a presence, absence, or relative amount) of sequences at each of a panel of condition-associated genomic loci in the sample may be indicative of the disease or disorder (e.g., a lupus, psoriasis, atopic dermatitis, and/or systemic sclerosis (scleroderma) condition) of the subject. The probes may be selective for the sequences at the panel of condition-associated genomic loci in the sample. A kit may comprise instructions for using the probes to process the sample to generate datasets indicative of a quantitative measure (e.g., indicative of a presence, absence, or relative amount) of sequences at each of the panel of condition-associated genomic loci in a sample of the subject.

The probes in the kit may be selective for the sequences at the panel of condition-associated genomic loci in the sample. The probes in the kit may be configured to selectively enrich nucleic acid (e.g., RNA or DNA) molecules corresponding to the panel of condition-associated genomic loci. The probes in the kit may be nucleic acid primers. The probes in the kit may have sequence complementarity with nucleic acid sequences from one or more of the panel of condition-associated genomic loci. The panel of condition-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct condition-associated genomic loci.

The instructions in the kit may comprise instructions to assay the sample using the probes that are selective for the sequences at the panel of condition-associated genomic loci in the cell-free biological sample. These probes may be nucleic acid molecules (e.g., RNA or DNA) having sequence complementarity with nucleic acid sequences (e.g., RNA or DNA) from one or more of the plurality of panel of condition-associated genomic loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the sample to generate datasets indicative of a quantitative measure (e.g., indicative of a presence, absence, or relative amount) of sequences at each of the panel of condition-associated genomic loci in the sample. A quantitative measure (e.g., indicative of a presence, absence, or relative amount) of sequences at each of a panel of condition-associated genomic loci in the sample may be indicative of a disease or disorder (e.g., a lupus, psoriasis, atopic dermatitis, and/or systemic sclerosis (scleroderma) condition).

The instructions in the kit may comprise instructions to measure and interpret assay readouts, which may be quantified at one or more of the panel of condition-associated genomic loci to generate the datasets indicative of a quantitative measure (e.g., indicative of a presence, absence, or relative amount) of sequences at each of the panel of condition-associated genomic loci in the sample. For example, quantification of array hybridization or polymerase chain reaction (PCR) corresponding to the panel of condition-associated genomic loci may generate the datasets indicative of a quantitative measure (e.g., indicative of a presence, absence, or relative amount) of sequences at each of the panel of condition-associated genomic loci in the sample. Assay readouts may comprise quantitative PCR (qPCR) values, digital PCR (dPCR) values, digital droplet PCR (ddPCR) values, fluorescence values, etc., or normalized values thereof. Various systems, methods, classifiers and kits are described in WO 2020/102043, which is entirely incorporated herein by reference.

In some embodiments, the dataset comprises RNA gene expression or transcriptome data, DNA genomic data, or a combination thereof. In some embodiments, the biological sample comprises a whole blood (WB) sample, a PBMC sample, a tissue sample, a cell sample or any derivative thereof. In some embodiments, assessing the SLE condition of the subject comprises determining a diagnosis of the SLE condition, a prognosis of the SLE condition, a susceptibility of the SLE condition, a treatment for the SLE condition, or an efficacy or non-efficacy of a treatment for the SLE condition.

In some embodiments, the method further comprises determining a diagnosis of the SLE condition with a sensitivity of at least about 70%. In some embodiments, the method further comprises determining a diagnosis of the SLE condition with a specificity of at least about 70%. In some embodiments, the method further comprises determining a diagnosis of the SLE condition with a positive predictive value of at least about 70%. In some embodiments, the method further comprises determining a diagnosis of the SLE condition with a negative predictive value of at least about 70%. In some embodiments, the method further comprises determining a diagnosis of the SLE condition with an Area Under Curve (AUC) of at least about 70%. In some embodiments, the method further comprises determining a likelihood of the diagnosis of the SLE condition of the subject.

In some embodiments, the method further comprises generating a plurality of drug candidates for the SLE condition of the subject. In some embodiments, the method further comprises evaluating or predicting a relative efficacy of the plurality of drug candidates for the SLE condition of the subject. In some embodiments, the method further comprises providing a therapeutic intervention comprising one or more of the plurality of drug candidates for the SLE condition of the subject.

In some embodiments, the method further comprises monitoring the SLE condition of the subject, wherein the monitoring comprises assessing the SLE condition of the subject at each of a plurality of time points, and processing the plurality of assessments of the SLE condition of the subject at each of the plurality of time points.

### EXAMPLES

The following illustrative examples are representative of embodiments of the software applications, systems, and methods described herein and are not meant to be limiting in any way.

### Example 1: Altered Expression of Genes Controlling Metabolism Characterizes the Tissue Response to Immune Injury in Lupus

In an aspect, the present disclosure provides systems and methods for using bioinformatics approaches to deconvolute bulk mRNA for various cells and processes involved in lupus organ pathology, including inflammatory cells, endothelial cells, tissue cells.

In an aspect, the present disclosure provides systems and methods for the delineation of the altered metabolism of cells by using gene expression analysis.

In an aspect, the present disclosure provides systems and methods for using various regression models (e.g., classification and regression trees, linear regression, step-wise regression) to dissect the specific metabolic alterations in individual cell types.

In an aspect, the present disclosure provides systems and methods for using animal models and the ability to translate mouse gene expression into the human equivalent to confirm the results in humans and also analyze the effects of treatment.

In an aspect, the present disclosure provides systems and methods for the delineation of the role of specific cells (myeloid cells) and processes (interferon, mitochondrial dysfunction) in lupus tissue pathology.

In an aspect, the present disclosure provides systems and methods for using non-lymphocyte populations in skin and kidney toward diagnostic and/or prognostic biopsy tests.

In an aspect, the present disclosure provides systems and methods for defining gene signatures in individual cell types in a mixed population such as blood or tissue (e.g., skin, kidney).

In an aspect, the present disclosure provides systems and methods for analyzing sets of metabolism genes and their relationship to function and cell type, including subsets of myeloid cells (e.g., subsets of myeloid celis new).

To compare lupus pathogenesis in disparate tissues, we analyzed gene expression profiles of human discoid lupus erythematosus (DLE) and lupus nephritis (LN). We found common increases in myeloid cell-defining gene sets and decreases in genes controlling glucose and lipid metabolism in lupus-affected skin and kidney. Regression models in DLE indicated increased glycolysis was correlated with keratinocyte, endothelial, and inflammatory cell transcripts, and decreased tricarboxylic (TCA) cycle genes were correlated with the keratinocyte signature. In LN, regression models demonstrated decreased glycolysis and TCA cycle genes were correlated with increased endothelial or decreased kidney cell transcripts, respectively. Less severe glomerular LN exhibited similar alterations in metabolism and tissue cell transcripts before monocyte/myeloid cell infiltration in some patients. Additionally, changes to mitochondrial and peroxisomal transcripts were associated with specific cells rather than global signal changes. Examination of murine LN gene expression demonstrated metabolic changes were not driven by acute exposure to type I interferon and may be restored after immunosuppression. Finally, expression of HAVCR1, a tubule damage marker, was negatively correlated with the TCA cycle signature in LN models. These results indicate that altered metabolic dysfunction is a common, reversible change in lupus-affected tissues and appears to reflect damage downstream of immunologic processes.

Systemic lupus erythematosus (SLE) is a complex autoimmune disease that affects multiple tissues within the body, including the skin and kidneys [1,2]. Although the primary mechanisms of SLE pathogenesis involve hyperactivity of both the innate and adaptive immune systems, evidence surrounding the involvement of perturbed metabolic activity has recently emerged [3-5]. Whereas systemic metabolic dysregulation has been associated with lupus-related morbidities, such as atherosclerosis [6], the contribution of cellular metabolic abnormalities in human lupus-affected tissues has yet to be fully explored. Metabolic derangements in tissues and their contributions to disease pathology have been investigated in a number of inflammatory and rheumatic diseases. For example, abnormalities in mitochondrial functions contribute to immune/inflammatory skin diseases [7], and skin cells have been shown to upregulate the pentose phosphate pathway (PPP) under oxidative stress [8]. Moreover, rheumatoid arthritis synoviocytes shift their metabolism to glycolysis because of local hypoxia [9] and osteoarthritis exhibited increased synovial tricarboxylic acid (TCA) cycle intermediates [10]. Similarly, metabolic impairment has been found in many forms of kidney disease [11,12], including defects in fatty acid oxidation (FAO) that have been correlated with fibrosis progression in the kidney tubulointerstitium [11].

At the cellular level, metabolic abnormalities in inflammatory diseases may be related to the nature of the immune cell infiltrate and its activation status. For example, macrophage polarization is regulated by glycolysis and FAO [13], and T cells reallocate glucose and upregulate glycolysis following activation [14]. Moreover, macrophage markers have been associated with increased PPP activity in kidney diseases, including lupus nephritis (LN) [15]. Additionally, exhausted T cells with altered mitochondrial function have been found in murine LN [16], and T cells isolated from SLE patients exhibit alterations in lipid composition [17]. The idea of targeting T cell metabolism for lupus therapy has been advocated [18,19] based on the finding that CD4 T cells in lupus-prone mice had elevated glycolysis and oxidative metabolism that may be normalized with metformin and 2-Deoxy-D-glucose (2DG) treatment resulting in disease improvement [4].

Taken together, these studies suggest metabolic abnormalities in either infiltrating inflammatory cells or tissue cells contribute to and/or reflect tissue damage. To examine this in greater detail, we analyzed gene expression profiles in human and murine lupus tissues to discern the nature of abnormal metabolic pathways, elucidate cellular origins of metabolic abnormalities, and determine how inflammatory cells contribute to physiologic processes involved in tissue inflammation and damage.

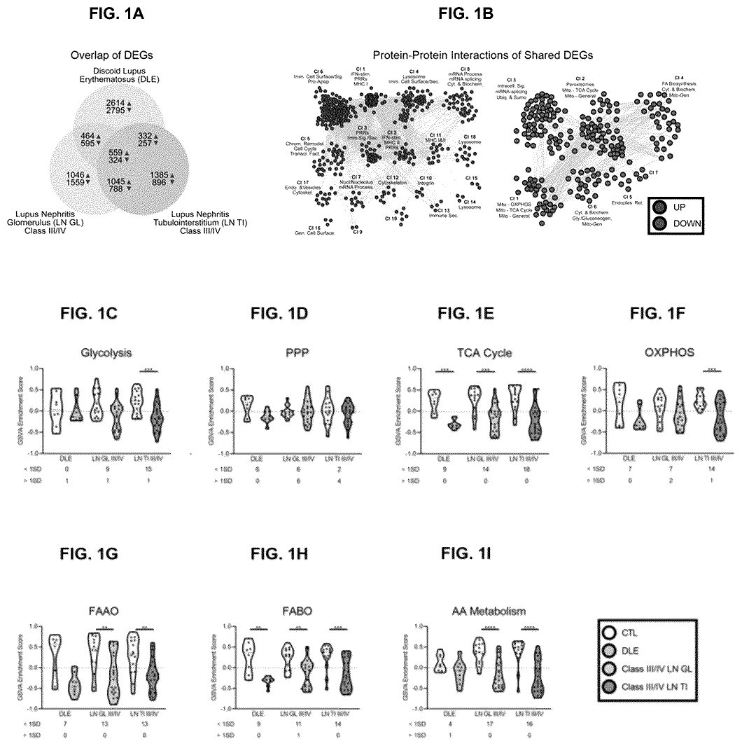

Dysregulation of metabolic gene signatures was found to be common among lupus-affected tissues. Despite thousands of differentially expressed genes (DEGs) in discoid lupus erythematosus (DLE), World Health Organization (WHO) class III/IV LN glomerulus (GL) and WHO class III/IV LN tubulointerstitium (TI), there were only 559 increased and 324 decreased transcripts in common (FIG. 1A). Protein-protein interaction analysis showed the common upregulated genes were related to interferon (IFN) and immune signaling pathways (FIG. 1B). The common downregulated genes were related to mitochondrial processes, including the TCA cycle and oxidative phosphorylation (OXPHOS).

As there is considerable transcriptomic heterogeneity among lupus patients [20,21], we sought to examine expression of genes controlling metabolism at the individual patient level, and, therefore, employed gene set variation analysis (GSVA) [22](Table 2). Generally, lupus tissues exhibited lower GSVA scores indicative of metabolic pathways, whereas controls had higher metabolism GSVA scores (FIGS. 1C-1I). The glycolysis and OXPHOS signatures were decreased in LN TI, whereas the TCA cycle and fatty acid beta oxidation (FABO) gene signatures were decreased in all tissues. Decreases in genes signifying fatty acid alpha oxidation (FAAO) and amino acid (AA) metabolism were similarly detected in the majority of lupus patients.

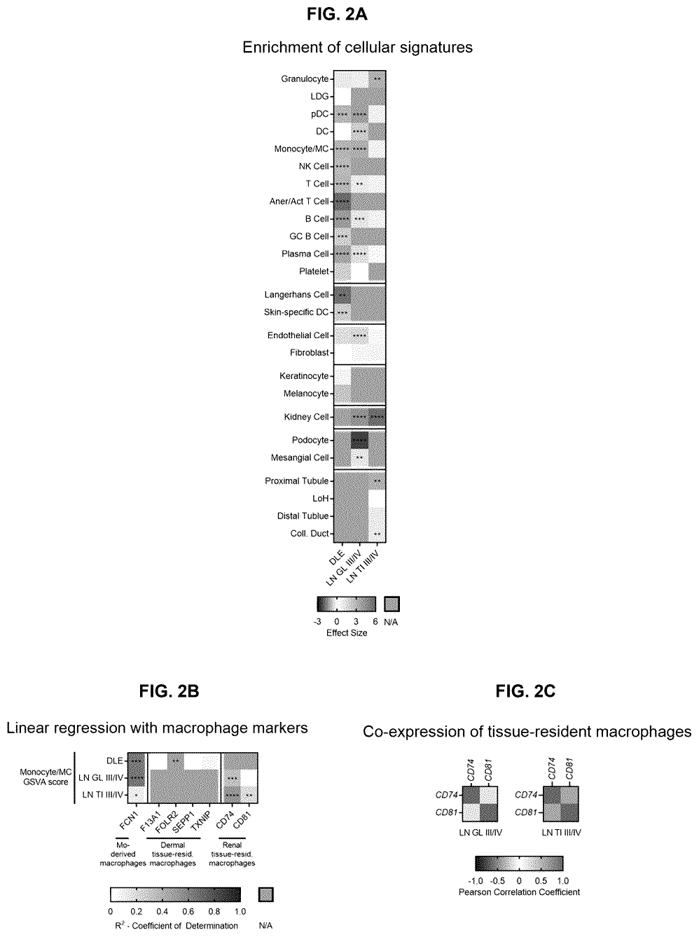

It was found that increased myeloid cell signatures and decreased tissue cell signatures characterize the majority of lupus patients. To determine whether cellular changes accounted for the decreased metabolic signatures, we examined enrichment of immune and non-hematopoietic cell signatures in lupus tissues. Increased tissue enrichment of immune cell signatures was variable, with DLE and LN GL demonstrating more enrichment of inflammatory cell signatures as compared to LN TI (FIGS. 2A, FIGS. 9A-90, FIG. 10). There was evidence of increased plasmacytoid dendritic cell (pDC), dendritic cell (DC), or monocyte/myeloid cell (monocyte/MC) signatures in some patients from each tissue. Non-hematopoietic cell gene expression was additionally altered. The endothelial cell (EC) signature was increased in LN GL. Keratinocyte transcripts were increased in 4/9 DLE patients, whereas melanocyte transcripts were decreased in 5/9 patients. General kidney cell transcripts were decreased in the majority of LN patients. Genes indicative of podocytes were decreased, whereas genes indicative of mesangial cells were increased in LN GL. Finally, the proximal tubule signature was significantly decreased and the collecting duct signature was significantly increased in LN TI.

Although the monocyte/MC signature was consistently increased among lupus-affected tissues, its nature in each tissue varied. Linear regression of the monocyte/MC signature and FCN1 expression indicated DLE and LN GL contained inflammatory monocyte-derived macrophages [23], but the correlation in LN TI was weak (FIG. 2B, Table 2). Tissue-resident macrophage populations were evaluated in DLE by expression of F13A1, FOLR2, SEPP1, and TXNIP [24] and in LN by co-expression of CD74 and CD81, which is characteristic of renal tissue-resident macrophages [25]. The tissue-resident macrophage signature was present in LN TI as evidenced by monocyte/MC correlation with both markers and co-expression of CD74 and CD81 (FIGS. 2B-2C, FIG. 11B). However, the tissue-resident macrophage signature was not identified in DLE as shown by lack of correlation with F13A1, SEPP1, and TXNIP. Similarly, the tissue-resident macrophage signature was not found in LN GL as shown by lack of correlation with CD81 and no co-expression between CD74 and CD81. This suggests monocyte/MCs in DLE and LN GL are predominantly infiltrating inflammatory monocyte-derived macrophages, whereas LN TI is populated by tissue-resident macrophages.

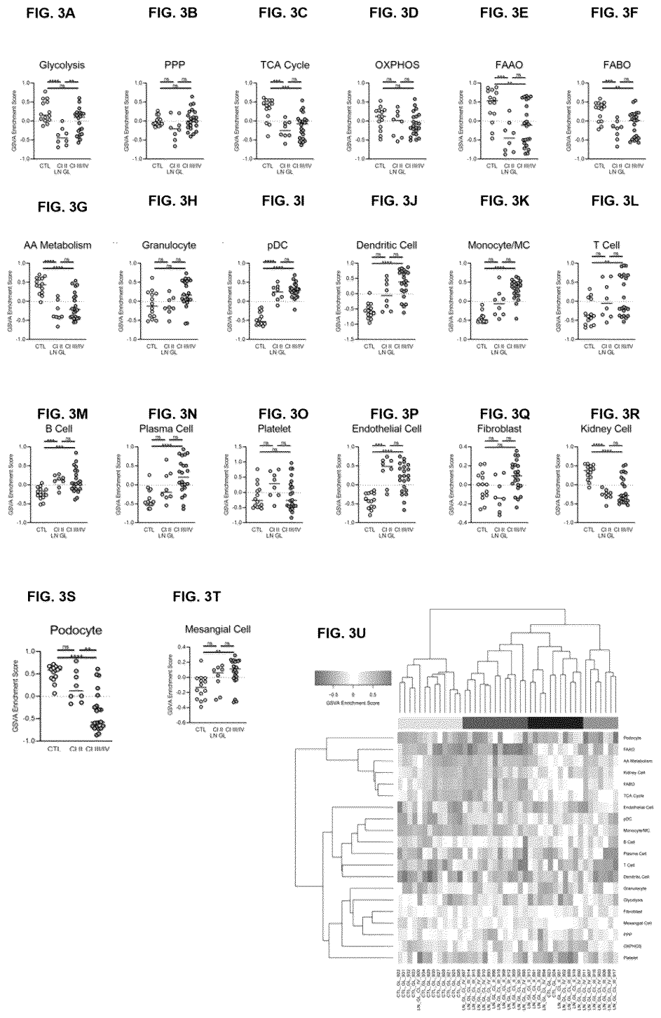

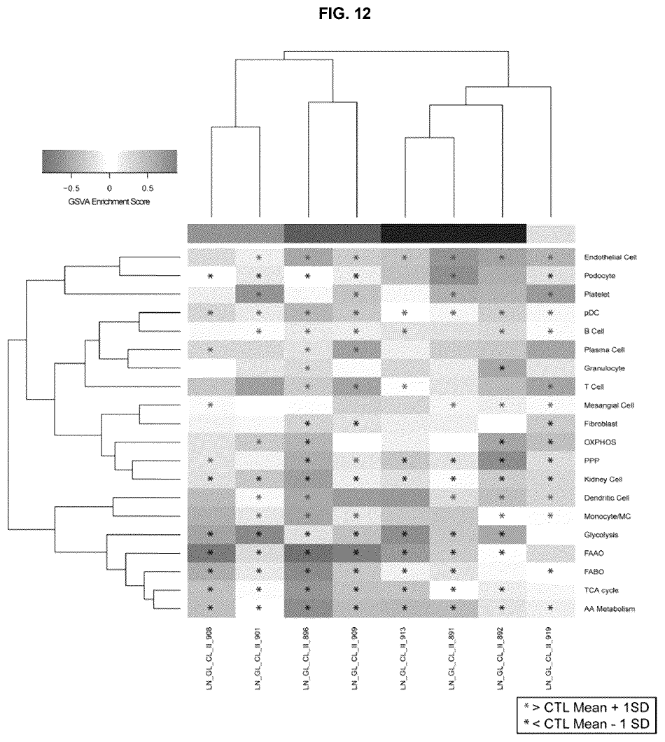

It was found that Class II LN GL is molecularly similar to class III/IV LN GL. To elucidate whether the same metabolic signature changes were present in less severe LN, we expanded our analysis to incorporate WHO class II LN samples, where less immune cells have been observed histologically [26]. Unexpectedly, we found that genes controlling glycolysis, the TCA cycle, FAO, and AA metabolism were decreased in class II LN GL (FIGS. 3A-3G), and gene expression of pDCs and B cells was increased in class II (FIGS. 3H-30). It is notable that gene expression of non-hematopoeitic cell populations was altered in class II LN GL, including a decrease in kidney cell genes and an increase in EC genes (FIGS. 3P-3T). These abnormalities in expression of metabolic genes and non-hematopoeitic cell genes were noted even in patients in which an increase in monocyte/MC genes was not detected (FIG. 12). Altogether, even though inflammatory cell gene signatures were less pronounced in some patients relative to class III/IV, class II LN GL was molecularly similar to class III/IV LN GL (FIG. 3U).

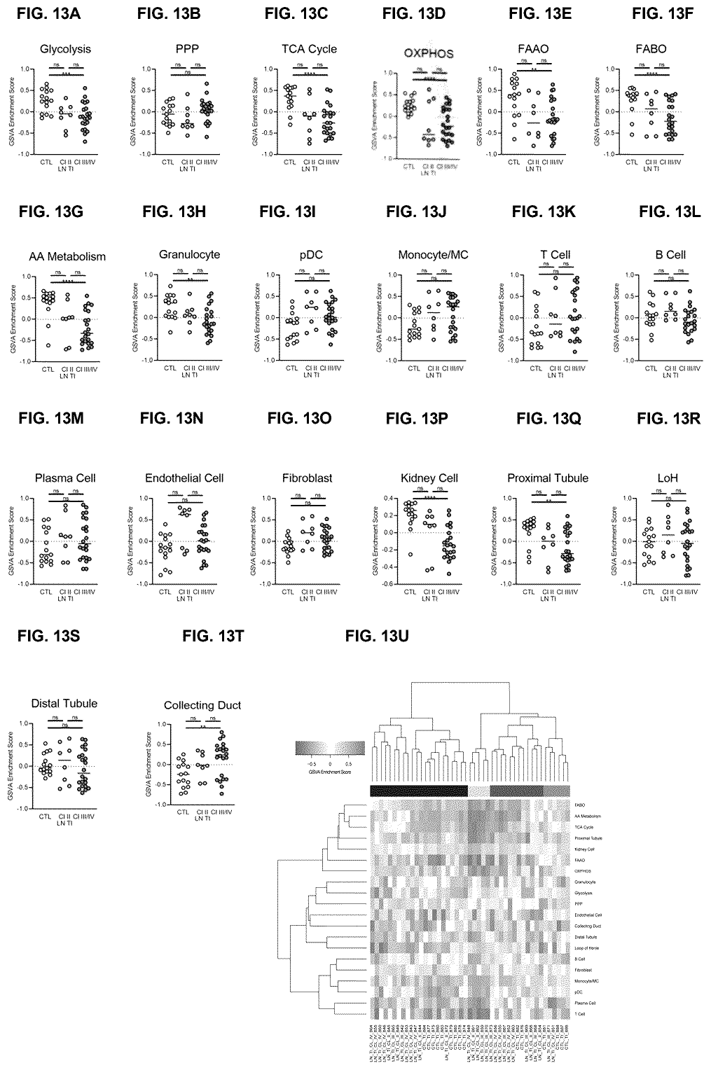

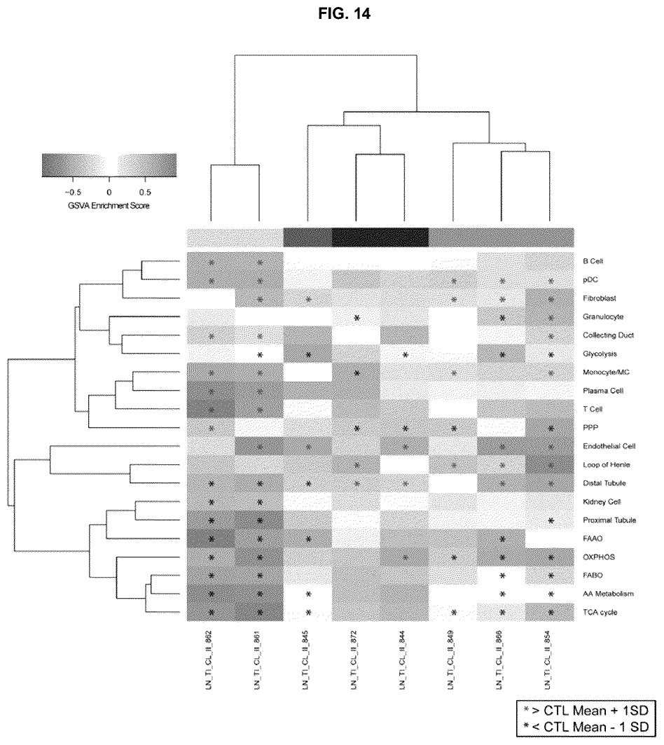

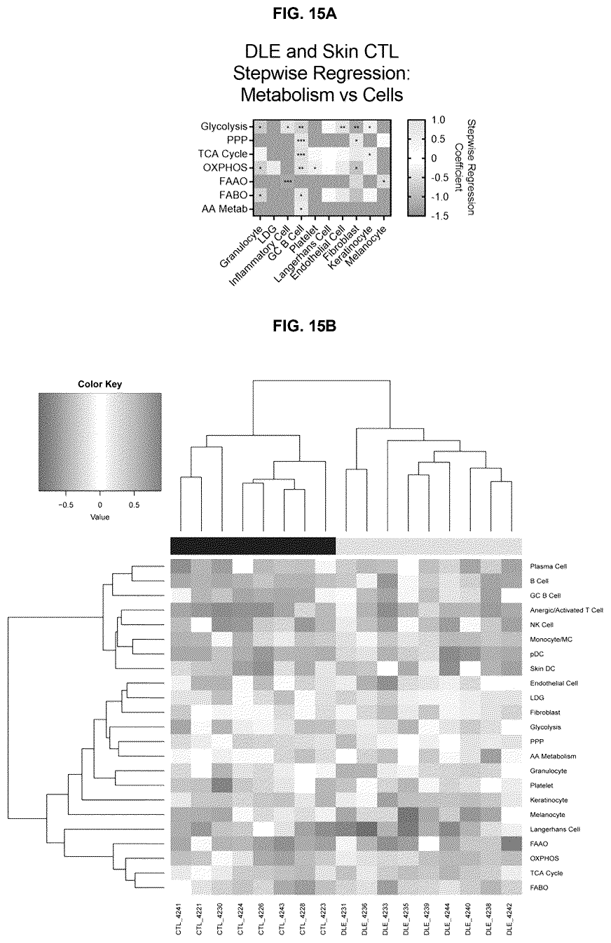

As in the glomerulus, class II LN TI was not statistically different from class III/IV LN TI (FIGS. 13A-13U); however, class II LN TI did not display decreased metabolic signatures. Class II LN TI had less indication of tissue cell damage and inflammatory infiltrate than class II LN GL (FIG. 14).

It was found that cellular signatures are associated with metabolic gene signature dysregulation in lupus-affected tissues. Stepwise regression, which identifies the independent variables that best explain the dependent variable [27], was employed to analyze cellular signature associations with metabolism gene signature changes. To improve precision, highly collinear cellular signatures in DLE were combined for stepwise analysis. In DLE, the inflammatory cell, EC, and keratinocyte signatures were positively correlated with the glycolysis signature, as indicated by a positive regression coefficient (FIGS. 15A-15B). Conversely, the GC B cell and fibroblast signatures were negatively correlated with other metabolic signatures. The keratinocyte signature was also negatively correlated with the TCA cycle signature.

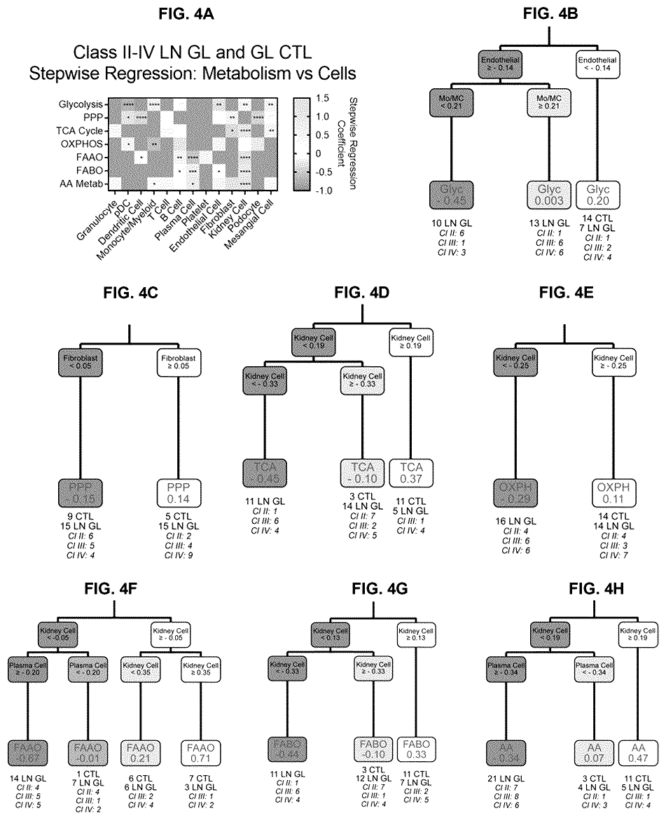

We then implemented both stepwise regression and classification and regression tree (CART) analysis, which partitions data by independent variables [28], to determine the cellular signatures that were most associated with the metabolic signature changes in all classes of LN and controls. In LN GL, all metabolic signatures except for the PPP signature exhibited some dependence on the kidney cell signature by either stepwise regression or CART (FIGS. 4A-4H). The stepwise regression coefficients for the kidney cell signature were positive and of the largest magnitude for the TCA cycle, FAAO, FABO, and AA metabolism signatures; a negative regression coefficient was noted for the EC signature and glycolysis. Moreover, CART identified the EC signature as the strongest contributor to the glycolysis signature, with a positive EC GSVA score predicting a lower glycolysis GSVA score. Importantly, the EC signature classifier alone separated 23 of 30 LN GL samples from controls. It is notable that by CART analysis upregulation of the monocyte/MC signature tended to mitigate the decreased glycolysis signature related to ECs, suggesting that monocyte/MC genes might contribute to glycolysis in opposite directions from EC genes. The monocyte/MC signature exhibited a negative stepwise regression coefficient with the OXPHOS signature, which was also positively associated with the kidney cell signature by CART. Together, the stepwise regression and CART models indicate decreased OXPHOS is reflective of decreased kidney cell and increased monocyte/MC signatures, although increased pDC genes may also contribute.

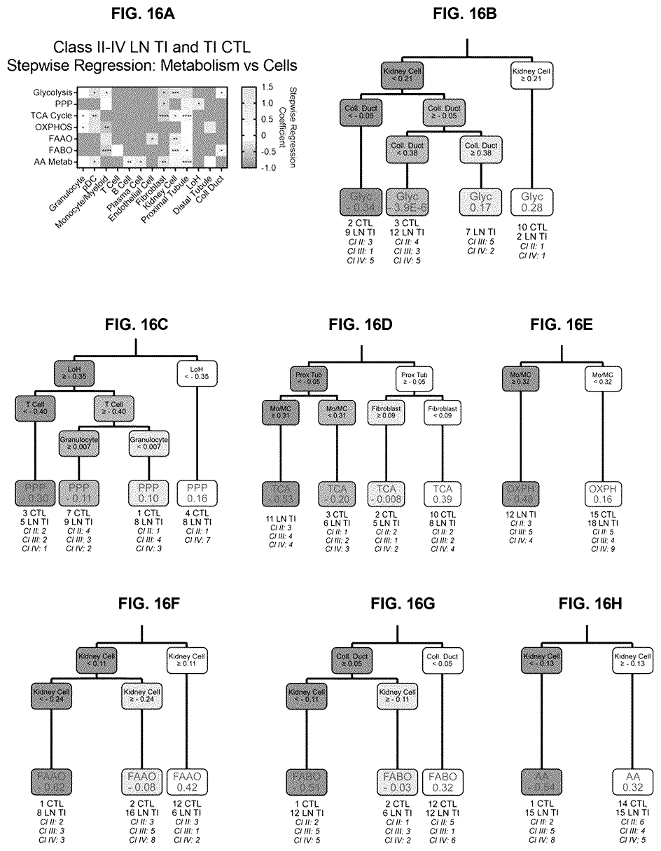

The contribution of kidney-specific cell signatures to most metabolic changes was also observed in the TI (FIGS. 16A-16H). In contrast to LN GL, EC genes did not contribute to the decreased glycolysis signature in LN TI, but instead the kidney cell signature was predicted by stepwise regression and CART. Numerous cell types were found to contribute to the overall TCA cycle signature. Stepwise regression demonstrated a positive contribution from proximal tubule, kidney cell, and granulocyte transcripts, and a negative contribution from fibroblast and pDC cell signatures. CART identified the proximal tubule signature as the strongest contributor, with a positive correlation to the TCA cycle signature, and confirmed the involvement of the fibroblast signature as well. Monocyte/MC transcripts contributed most to the OXPHOS signature, although the regression coefficient was negative, implying that the presence of monocyte/MC contributed to a decreased OXPHOS signature. The FABO signature, which may be defective in affected TI of multiple etiologies [11,12,29], FAAO, and AA metabolism signatures also demonstrated a positive relationship with expression of kidney cell and/or proximal tubule genes.

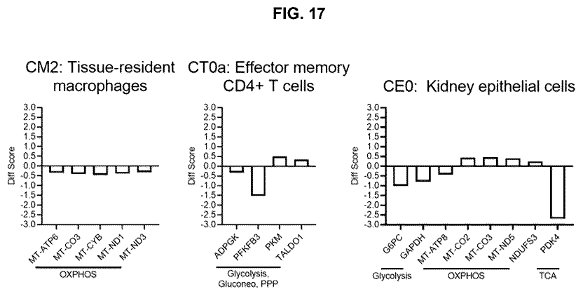

We sought to confirm these findings by referencing data from single-cell RNA-sequencing (scRNA-seq) of LN biopsies [30]. In LN, tissue-resident macrophages exhibited decreased OXPHOS genes (FIG. 17), which aligns with the negative relationship that stepwise regression and CART predicted between the monocyte/MC and OXPHOS signatures in LN TI (FIGS. 16A and 16E). Conversely, CD4 T cells had both increased and decreased expression of glycolysis genes, making their metabolism difficult to interpret. The kidney epithelial cell cluster, which was comprised of general kidney cell as well as tubule genes, exhibited significant negative differential expression of two glycolysis genes (G6PC, GAPDH) and one TCA cycle gene (PDK4), but four OXPHOS genes were significantly over-expressed (MT-C02, MT-C03, MT-ND5, NDUFS3). This supports the idea that kidney cells have high oxidative metabolism, and their absence or dysfunction may contribute to decreased OXPHOS signatures. However, insufficient genes were detected by scRNA-seq to confirm the current findings determined with bulk RNA analysis.

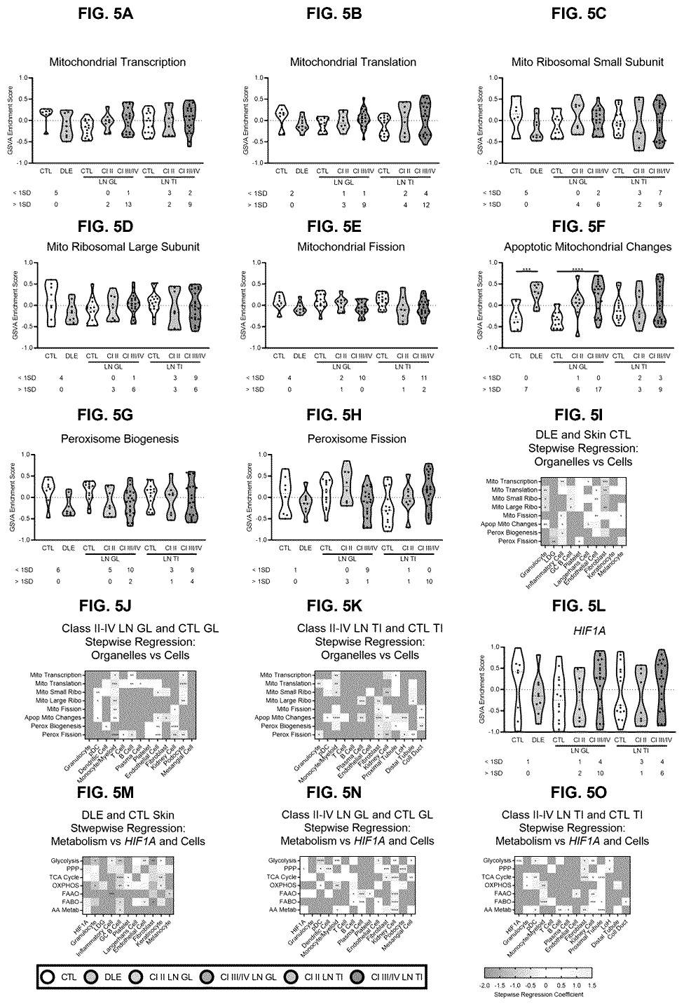

It was found that mitochondrial and peroxisomal signature changes and local hypoxia contribute to changes in metabolic gene expression in specific cells. As mitochondria and peroxisomes are the primary organelles responsible for metabolic processes such as OXPHOS and FAO [31], we sought to examine whether there were detectable changes to organelle-specific gene expression that may explain the altered metabolic state. There was no evidence of global mitochondrial gene expression changes, although mitochondrial genes were decreased in approximately half of DLE patients, and mitochondrial transcription was increased in 15/30 LN GL patients (FIGS. 5A-5E). Conversely, the apoptotic mitochondrial changes signature was increased in both DLE and LN GL (FIG. 5F). Genes associated with peroxisome biogenesis were decreased in some lupus patients in each tissue; in contrast, genes associated with peroxisomal fission were increased in some class III/IV LN TI patients (FIGS. 5G-5H). Stepwise regression analysis revealed a positive association between the apoptotic mitochondrial signature and the granulocyte and inflammatory cell signatures in DLE, and positive associations between the apoptotic mitochondrial signature and MC signatures in LN (FIGS. 5I-5K). Positive associations were observed between the peroxisome biogenesis signature and the kidney cell and proximal tubule signatures in LN GL and LN TI, respectively.

Since hypoxia has been cited as a driver of kidney disease in the TI [32], and a hypoxic microenvironment may result in decreased oxidative metabolism, we examined the contribution of HIF1A to metabolic gene signatures. Some lupus patients had increased expression of HIF1A by GSVA (FIG. 5L). Although HIF1A expression did not significantly affect metabolic signatures in DLE, in LN TI, the HIF1A gene signature had a positive correlation with the glycolysis signature, whereas in LN GL, the HIF1A gene signature was positively associated with the PPP signature (FIGS. 5M-5O). This suggests hypoxia contributes to specific metabolic alterations in the kidney.

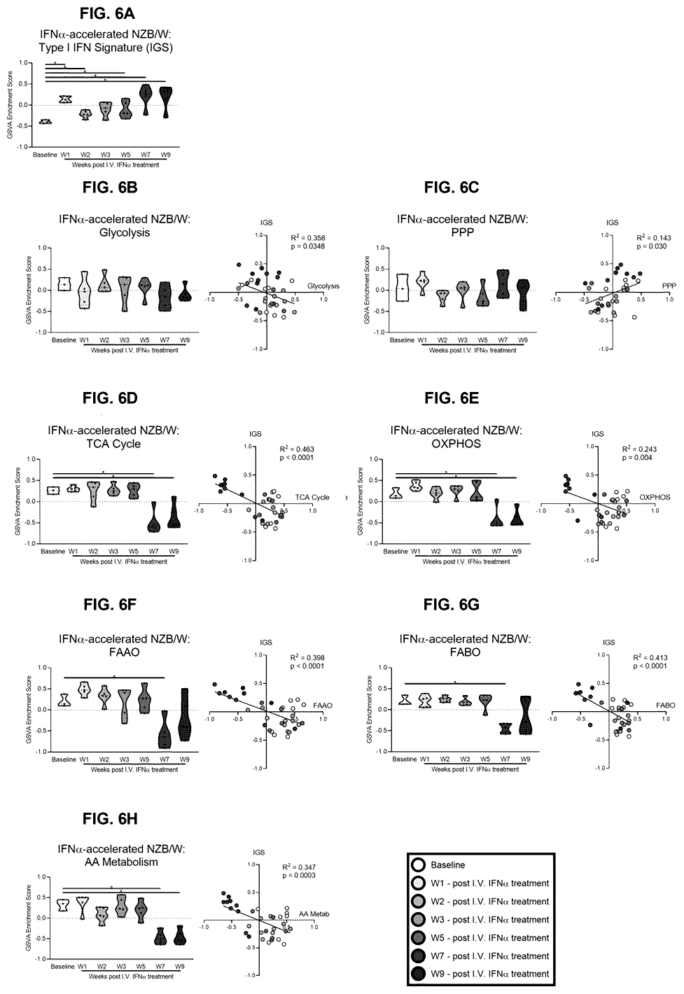

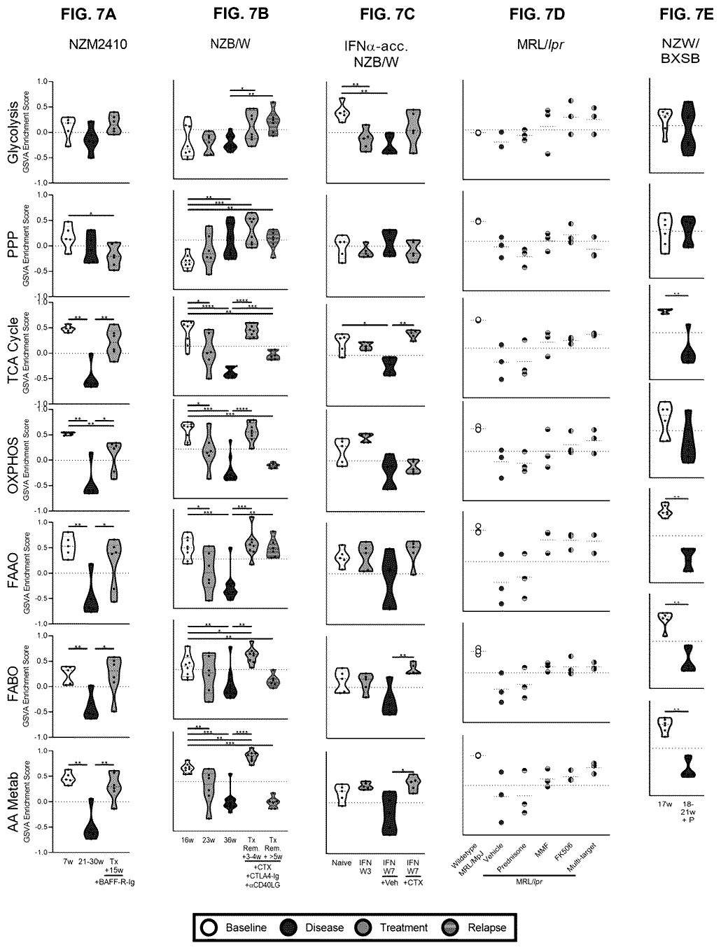

It was found that metabolic gene expression changes occur independent of acute IFN stimulation. The IFN gene signature (IGS), a known hallmark of lupus [33,34] and lupus-affected tissues [35], has been implicated in metabolic alteration of MCs [36-39]. To determine the functional relationship between IFN stimulation and metabolic alteration, we examined metabolic gene expression longitudinally in the IFNα-accelerated NZB/W murine model of LN, where the IGS increases at early timepoints following injection of IFNα adenovirus and then increases again when kidney disease develops (FIG. 6A). To determine whether metabolic signatures were changed with the elevated IGS, we examined metabolic signatures after IFNα administration. No significant changes in metabolic signatures were observed at week 1 (W1) after IFNα administration (FIGS. 6B-6H), the peak of early IGS expression. In contrast, the TCA cycle, OXPHOS, FAO, and AA metabolism gene signatures were decreased at W7 post-IFNα administration, when LN develops [40]. There were negative correlations between the IGS and metabolic signatures for the TCA cycle, OXPHOS, FAO, and AA metabolism, suggesting increased IFN may contribute to decreased metabolism. However, these is a clear separation of the W1 and W7/W9 datapoints in the correlation analysis for TCA cycle, OXPHOS and AA metabolism signatures, in which W1 datapoints show positive GSVA scores for both the IGS and metabolic signatures. Thus, based upon the lack of transcriptional metabolic changes following early IFN exposure in the mouse, it does not appear that acute exposure to type I IFN explains the metabolic changes in lupus tissues, as transcriptional changes to metabolism occur when disease develops and the IFN signature reoccurs, but not at W1, W2, or W3.

It was found that metabolic and cellular gene expression changes in murine LN are corrected by immunosuppressive treatment. To determine the robustness of the observed metabolic and cellular gene expression changes and determine whether dysregulation may be reversed by immunosuppressive therapy, we analyzed gene expression in pre- and post-treatment kidneys of lupus mice. Although some metabolic changes had been identified in the kidneys of untreated and treated NZB/W and NZM2410 mice [41], no analysis of the nature of the affected cells was carried out.

Metabolic gene expression was significantly altered in NZM2410, NZB/W, IFNα-accelerated NZB/W (GSE72410), and NZW/BXSB mice. Treatment of NZM2410 mice with BAFF-R-Ig and proteinuric NZB/W mice with a combination of cyclophosphamide (CTX)+CTLA4-Ig+anti-CD154 restored TCA cycle, FAAO, FABO, and AA metabolism gene expression (FIGS. 7A-7B). Notably, in the NZB/W kidneys when combination therapy was discontinued and LN relapsed, some metabolic abnormalities recurred. In IFNα-accelerated NZB/W mice, both the glycolysis and TCA cycle signatures were significantly decreased with the onset of LN (IFN W7), and CTX treatment significantly restored TCA cycle gene expression to baseline pre-disease levels (FIG. 7C). In MRL/lpr mice, the same trends were observed, although the data did not achieve statistical significance (FIG. 7D). NZW/BXSB mice had decreased metabolic signatures for all processes except glycolysis, the PPP, and OXPHOS (FIG. 7E).

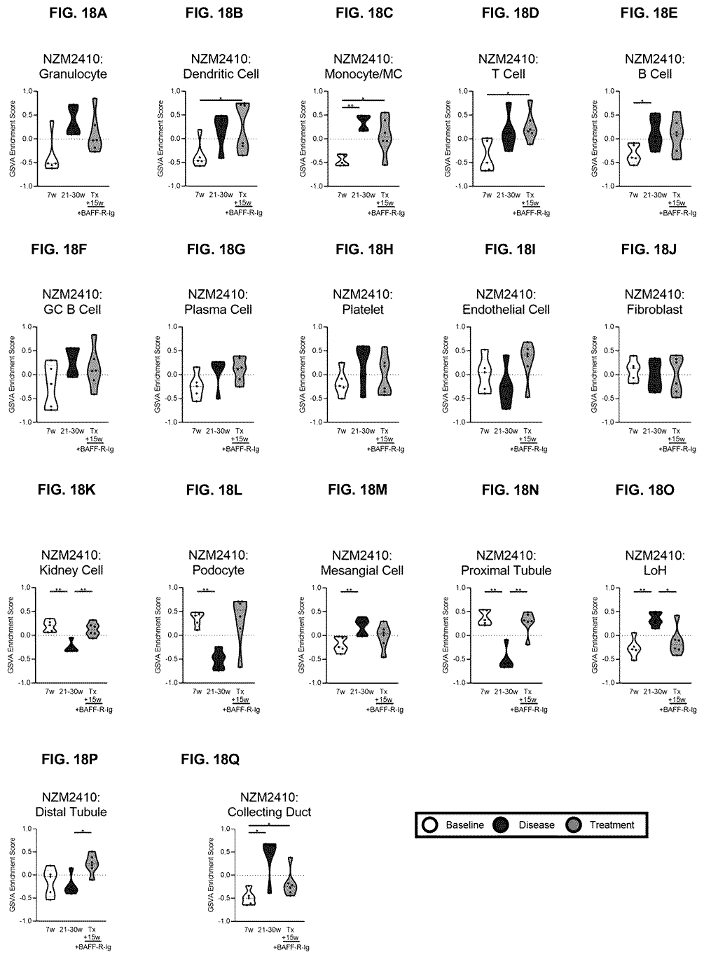

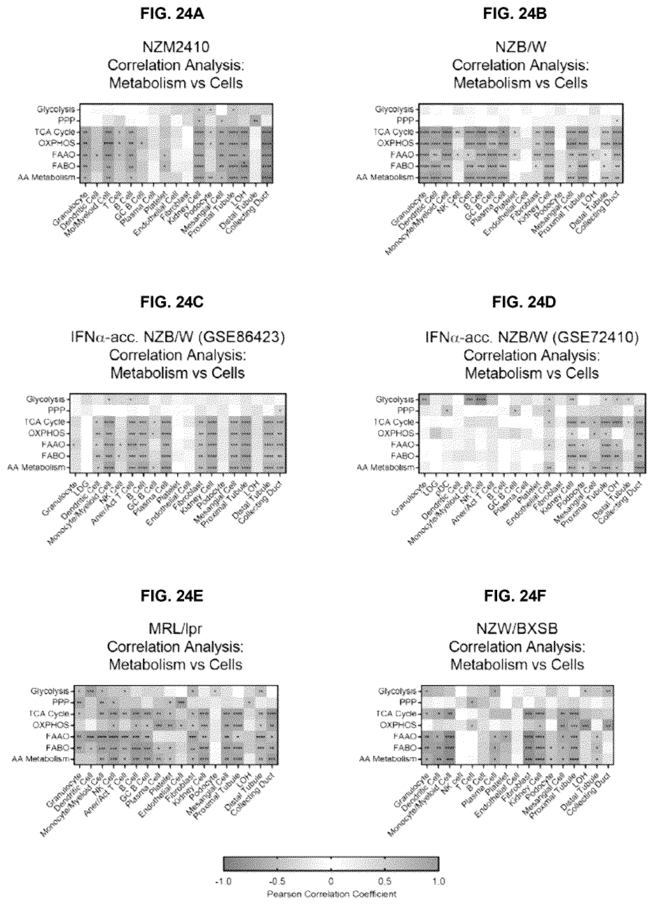

GSVA of cellular changes in murine LN models demonstrated similar results to human LN, although inflammatory infiltrate and changes in the EC and podocyte signatures were less robust (FIGS. 18A-18Q, FIGS. 19A-19R, FIGS. 20A-20S, FIGS. 21A-21S, FIGS. 22A-22R, FIGS. 23A-23Q). BAFF-R-Ig, combination therapy, and CTX-treatment restored kidney cell and proximal tubule gene signatures, and combination therapy decreased inflammatory cell gene signatures. Correlation analysis in all murine LN models showed strong positive correlations between metabolism and proximal tubule transcripts, whereas negative correlations were seen between metabolism and inflammatory cell signatures (FIGS. 24A-24F). This suggests that treatment may allow for functional metabolic recovery of non-hematopoietic cells in the kidney in the presence or absence of changes to the inflammatory cell signal.

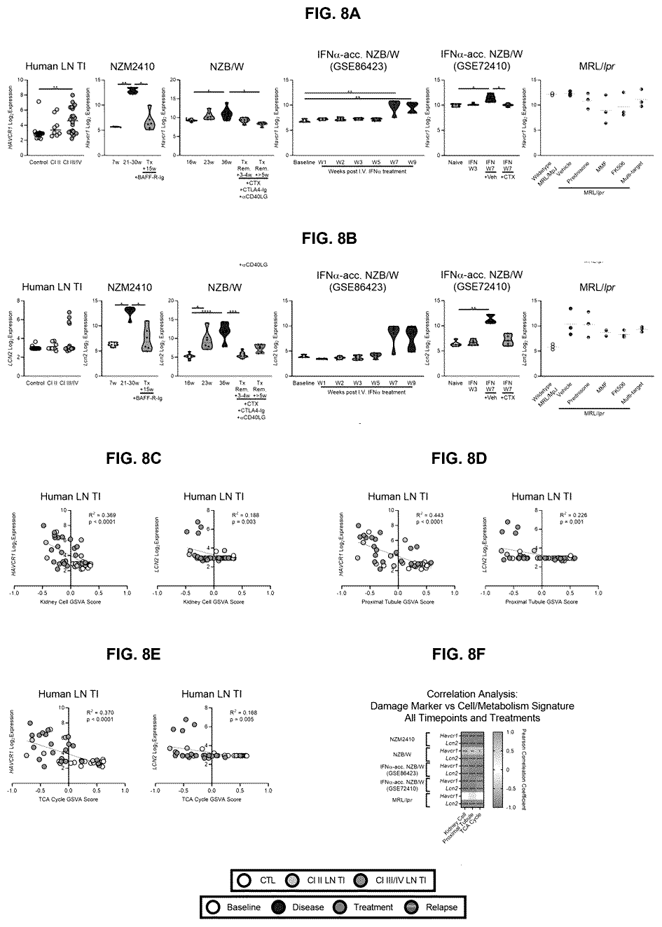

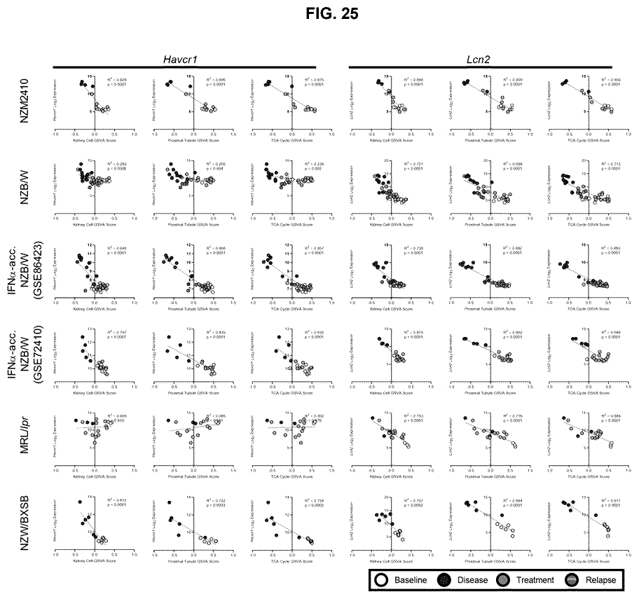

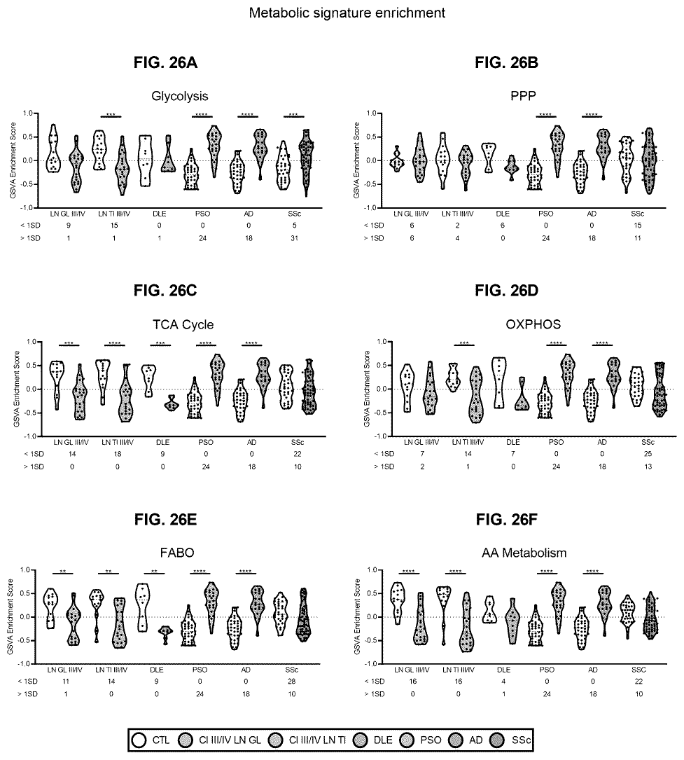

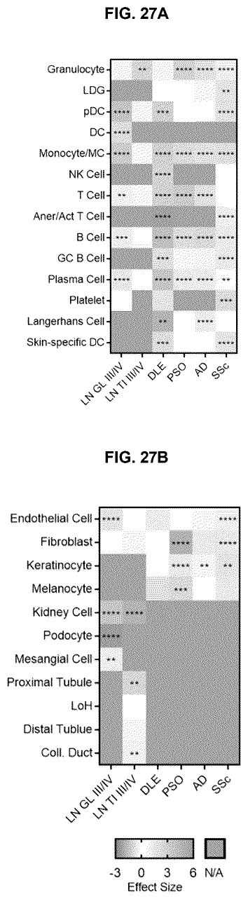

It was found that metabolic changes correlate with expression of genes indicating tubular damage. Finally, we examined whether changes in genes controlling metabolism occurred synchronously with changes in expression of HAVCR1 (KIM1) and LCN2, which are known markers of tubular damage [42,43]. Although HAVCR1 expression was increased in class III/IV human LN TI patients, there were no significant changes to LCN2 in any class of LN TI (FIGS. 8A-8B). Similarly, in murine LN models, Havcr1 and Len1 expression increased with active disease and returned toward normal with treatment. Both markers demonstrated significant inverse correlations with the kidney cell, proximal tubule, and TCA cycle signatures in both human and murine LN, although there were model-dependent differences in magnitude (FIGS. 8C-8F, FIG. 25).

Multi-pronged bioinformatic analyses of gene expression data from human lupus tissues revealed that despite intra-tissue heterogeneity metabolic dysfunction was present in all tissues. Immune effector cells have high metabolic needs [13,14,44,45] and, therefore, we initially hypothesized immune infiltration may be responsible for the observed lupus tissue-wide metabolic dysregulation. Although kidney-infiltrating CD8 T cells in murine LN are functionally exhausted with defective mitochondria [16], anergic/activated T cell markers were not found in these human LN samples, and regression models indicated T cells contributed minimally to changes in renal metabolic gene expression. Similarly, monocyte/MCs, which were increased in some patients from all tissues, might be expected to contribute to enhanced glucose metabolism—either glycolysis (M1 macrophages) or OXPHOS (M2 macrophages) [44]. Indeed, monocyte/MC signatures were inversely correlated with OXPHOS in both LN tissues, and positively correlated with glycolysis in LN GL, suggesting they are likely M1 in nature and may contribute to the altered metabolic landscape of intact tissues. Although gene expression revealed differing origins of the renal MC populations, as they reflect monocyte-derived macrophages in LN GL and tissue-resident macrophages in LN TI, the consistent MC presence aligns with their prominent role in tissue damage [46]. Observed increases in the monocyte/MC signature and strong inverse correlations between MCs and metabolism may reflect the role of MCs in tissue damage, even when T and B cells are not yet abundant.

To examine whether metabolic abnormalities represented primarily tissue cell defects as opposed to changes in inflammatory cell metabolism, we analyzed gene expression in class II LN, in which less inflammatory infiltrate is evident histologically [26]. Even though class II LN samples had evidence of increased immune/inflammatory cell signatures that coincided with changes to metabolic signatures, examples were observed in which the changes in metabolic signatures were found in the absence of a monocyte/MC signature, suggesting alterations in metabolic signatures may be initiated immediately following immune complex (IC) deposition and complement activation. Subsequent monocyte/MC and other inflammatory cell activation/infiltration may then contribute to further damage of tissue cells. Indeed, changes in kidney gene expression may occur following early IC deposition, but before microscopic detection of inflammation [47], consistent with our transcriptomic results in class II LN.

Our findings of altered metabolism in lupus tissues align with changes seen in other forms of tissue pathology. We observed a positive association between the keratinocyte and glycolysis signatures, and upregulation of glycolysis has been observed in keratinocytes during cutaneous infection [48]. Increased glycolysis [49,50] and decreased PPAR signaling, TCA cycle, and OXPHOS have been reported in dermal fibroblasts subjected to radiation [50], indicating dermal fibroblasts have the potential to contribute to inflammation-induced alterations in metabolism. However, in the current study, stepwise regression indicated that the fibroblast signature was negatively associated with the glycolysis, PPP, and OXPHOS signatures, whereas associations with FAO did not achieve statistical significance. The negative correlation between the fibroblast and PPP signatures contrasts with the observed upregulation of the PPP in cultured fibroblasts and keratinocytes that were exposed to UV-induced oxidative stress [8]. This suggests that in vivo fibroblasts are altered by signals different than that mediated by UV light, as expected since fibroblasts are deep in the dermis and shielded from such ambient exposure.

Metabolic dysregulation is also common in kidney disease [12,15,29,51,52]. In non-diabetic chronic kidney disease, TCA cycle abnormalities measured in urine metabolites coincided with changes to kidney gene expression [51], supporting our conclusions that metabolic dysregulation primarily reflects altered renal cell function as opposed to changes in immune cell metabolism. Moreover, defects in FAO have been correlated with fibrosis progression in TI disease [11]. Both human and murine models with TI fibrosis exhibited decreased expression of FAO enzymes and resultant increases in lipid deposition, which was reversed by correcting the metabolic abnormalities [11]. We similarly found that FAO signatures are substantially decreased in the TI; however, regression models indicated that altered FAO transcripts were most associated with decreased kidney cell signatures. Although fibroblasts were not predicted by the models, fibrosis or fibroblast enrichment may contribute to decreased kidney cell and proximal tubule transcripts in the TI.

In the glomerulus, ECs appear to play an additional role in disease. Endothelial activation in LN has been suggested [53], and EC transcripts were increased in 83% of all LN GL samples. Increased EC transcripts may reflect altered EC function, potentially resulting from cytokine/growth factor stimulation and/or hypoxia-induced cellular damage. Abnormal angiogenesis has been reported in diabetic nephropathy resulting in leaky vessels 54, but the function of glomerular ECs in LN is less well-defined. Indeed, glomerular ECs have been found to be dependent upon podocyte stimulation for differentiation [55], whereas other studies suggest EC damage precedes podocyte injury [56]. Our findings from class II LN support the latter, as signature changes to ECs occurred in the absence of podocyte changes in some patients, suggesting that ECs are early participants in LN.

The relationship between the EC signature and metabolic gene expression changes implied an alteration in EC physiology in LN. Although healthy ECs are highly glycolytic [57], all regression techniques in LN GL indicated an inverse correlation between the EC and glycolysis signatures. Consistent with our findings, in diabetic nephropathy stalled glycolytic flux has been observed in ECs [57]. These data suggest that glomerular ECs are metabolically altered, perhaps because of IC and complement stimulation and/or cytokine exposure, making them less capable of maintaining normal function. Indeed, in the GL, the EC signature had a positive regression coefficient with the FABO signature, and quiescent ECs have been reported to upregulate FABO [58], supporting the conclusion that ECs are functionally deranged in LN GL.

Analysis of metabolism-associated genes in cell clusters derived from scRNA-seq of LN biopsies [30] further supports our finding that changes in metabolism are most closely related to kidney cell gene expression, with minor contributions from resident or infiltrating immune cells. Tissue-resident macrophages exhibited decreased OXPHOS genes, whereas CD4 T cell metabolism was unclear. Notably, the kidney epithelial cell cluster reported many metabolism-associated genes, suggesting decreased glycolysis and TCA cycle, but increased OXPHOS. Proximal tubules, which have the most mitochondria of any kidney epithelial cell, are known to be dependent on oxidative metabolism [59], and this further supports the idea that diminished OXPHOS in bulk RNA in part reflects decreased kidney epithelial cell transcripts. Additionally, because scRNA-seq looks at expression of individual cells as opposed to the bulk environment, the detected kidney epithelial cells are likely the residual functionally normal ones. However, because of technical issues including cell yield and read depth, there are difficulties in determining the status of individual cell metabolism from the scRNA-seq data. Altogether, scRNA-seq appeared to be less effective than deconvolution of bulk RNA to detect important but subtle changes in cellular metabolism.

To determine whether defective organelles were responsible for metabolic alteration, we examined gene expression specific for both mitochondrial and peroxisomal function. We observed changes to the mitochondrial and peroxisomal gene signals in some patients, and correlation analysis suggested these changes were associated with specific cell types. Notably, the peroxisome biogenesis signature was positively associated with signatures for ECs, kidney cells, and proximal tubules. Moreover, as the kidney is highly susceptible to hypoxia [60], we investigated the propensity for hypoxia to contribute to altered metabolism. Although GSVA demonstrated no significant increases in HIF1A expression, there was an association between HIF1A and the PPP and glycolysis signatures in LN GL and LN TI, respectively, suggesting that hypoxia may have specific effects on metabolism, and may contribute to some of the metabolic changes observed in the tissues.

The relationship between the IGS and metabolic signature changes in IFNα-accelerated LN mice indicated that acute type I IFN exposure may not explain the observed metabolic changes. The mouse studies were particularly informative because IFNα exposure was regulated. Although it has been demonstrated that type I IFN stimulation may increase OXPHOS and FAO in DCs and MCs [36,38], inhibit isocitrate dehydrogenase (part of the TCA cycle) in macrophages [39], and alter oxidative metabolism in other cells [61], IFNα overexpression did not change metabolic signatures at early timepoints. However, there was an inverse relationship between the IGS and metabolic defects after LN onset, when the IGS recurred. These results support the conclusion that downregulation of metabolic pathways is unlikely to be explained by the known actions of type I IFN alone, but rather during LN progression, decreased metabolic signatures may be parallel reflections of continued IGS exposure and inflammation.

Metabolic gene expression was altered in four murine LN models and immunosuppressive treatment, not known to directly affect cellular metabolism, restored metabolic gene expression. Although combination therapy diminished inflammatory cell abundance in the kidneys of NZB/W mice, we also observed restoration of metabolic and kidney cell gene expression after treatment in models with little inflammatory infiltrate or those in which inflammatory cells were not significantly decreased with treatment. This suggests that although inflammatory cells play a critical role in mediating tissue cell damage and metabolic dysfunction, damage is not related only to local inflammatory cells, as intensive therapy with CTX restores tissue cell defects without significant changes to inflammatory cells. Importantly, these results demonstrate that metabolic abnormalities in tissue cells are reversible with immunosuppressive therapy and restored metabolic gene expression might be considered a goal of effective lupus treatment. Moreover, monitoring tissue metabolism may be especially important in situations where anti-metabolic drugs, such as metformin or 2DG, are employed. Whereas these drugs may be promising for correction of individual immune cell defects, they have potential consequences for already metabolically deranged tissue cells.

Additionally, these studies reveal subtle differences in pathology of glomerular and tubulointerstitial involvement in LN. In both kidney regions, we observed decreased resident non-hematopoietic cell signatures and increased monocyte/MC signatures. However, monocyte/MCs in the glomerulus were likely monocyte-derived, whereas those in the tubulointerstitium appeared more like tissue-resident macrophages. Moreover, tubulointerstitial diseases in class III/IV LN was characterized by less inflammatory infiltrate than was observed in the glomerulus, and although metabolic signatures were comparably decreased in all classes of glomerular LN, metabolic signature changes in class II tubulointerstitial LN were less consistently regulated. These results align with studies that show tubulointerstitial damage occurs later in LN and predicts end stage renal disease [62]. Poorer outcomes in class III/IV may be related to persistence of abnormalities or inhibition of repair mechanism that might contribute to progressive renal disease. Nonetheless, it is noteworthy that even the more modest immunologic damage in class II LN was associated with marked changes in metabolic signatures.

Detectable changes to immune cell, EC, kidney cell, and metabolic gene signatures in all classes of LN GL is notable. We found that gene expression may detect cellular changes with greater sensitivity than immunohistochemistry, when little or no inflammatory infiltrate is observed histologically. Gene expression may provide an advance to current classification or diagnostic techniques, as gene expression changes are detectable before discernable immunohistochemical changes, and transcriptomic analysis of metabolism may elucidate potential functional rather than merely histopathologic changes.

Our study is not without limitations. We performed post-hoc analysis of bulk gene expression in three lupus-affected tissues comprising limited numbers of lupus patients. Moreover, 37.5% of LN patients were being treated with immunosuppresives [53], that may have affected gene signatures. Additionally, the gene signature we identified as reflecting general kidney cells may be more specific for tubule cells, despite the strong representation in the glomerulus. Furthermore, regression analyses provided an estimate of the cellular variables that are most associated with each metabolic signature, but accuracy may be limited by sample size, and there is a chance of overfitting. Future work with larger cohorts currently not available would be necessary to validate these results. Additionally, direct assessment of functional metabolism may be necessary to assay how metabolic changes at the gene expression level reflect changes in protein content and cellular function.

In conclusion, prominent alterations in cellular metabolism signatures are characteristic of lupus tissue pathology. Systems bioinformatics and assessment with regression modeling techniques revealed that the monocyte/MC signature, including both monocyte-derived macrophages and tissue-resident macrophages, was increased in many lupus patients, kidney cell signatures were decreased in LN, the EC signature was increased in LN GL, and these cell signature changes were associated with altered metabolism signatures. Moreover, apoptotic mitochondrial gene changes were associated with MC genes in DLE and LN GL. In murine LN, metabolic dysregulation correlated with tubular damage marker expression and metabolic gene changes were reverted to normal by immunosuppressive therapy. Altogether, altered metabolism may serve as a promising biomarker or therapeutic target for lupus tissue disease, especially as metabolic gene expression changes precede expression of the renal damage biomarker LCN2 in human LN, and coincide with changes to HAVCR1. Indeed, urinalysis has been used to measure metabolite biomarkers in the kidney [51,52] and metabolism transcripts may be used to estimate degree of kidney cell damage and assess treatment efficacy. Although treatment strategies aimed at metabolic restoration are not straightforward, the current findings support the conclusion that immunosuppressive therapy may restore metabolic function, and, thereby, may ameliorate damage in specific lupus-affected tissues.

Human and mouse gene expression datasets were analyzed as follows. Raw data from publicly available human and murine lupus datasets were derived from the Gene Expression Omnibus (GEO) repository.

GSE72535 comprises microarray analysis of lesional skin biopsies from human patients with DLE with no systemic involvement with Cutaneous Lupus Activity and Severity Index (CLASI)≥2 [63]. Some DLE patients were treated with various therapies including corticosteroids, immunomodulators, and hydroxychloroquine [63].

GSE32591 comprises microarray analysis of human renal biopsies that were originally derived from the European Renal cDNA Bank (ERCB) [53]. LN patients from the ERCB (n=32) had an average age of 35.1±2.4 years, average proteinuria of 2.9±0.6 g/day, and eGFRMDRD of 63.7±5.4 ml/min/1.73 m2. LN patients were treated with various therapies, including steroids and immunosuppressants53. Two patients from this group were excluded from our analyses because they had non-inflammatory class V LN.

GSE86423 comprises samples from the IFNα-accelerated NZB/W LN model. Female NZB/W mice were injected with an adenovirus vector expressing recombinant murine IFNα (5×109 particles) at 9 weeks40. Kidney gene expression was measured in mice at 0, 1, 2, 3, 4, 5, 7, and 9 weeks post IFNα injection [40].

GSE32583 and GSE49898 comprise samples from three murine lupus models. Kidney gene expression was measured in NZM2410 mice including 6-8 week pre-disease mice, 22-30 week diseased mice, and treated mice in remission (Tx+15w) [41,53]. NZM2410 mice in the Tx+15w group were treated with adenovirus expressing BAFF-R-Ig at 22 weeks, and then sacrificed at 30-35 weeks or 55 weeks [41]. Kidney gene expression in NZB/W mice was measured at 16w, 23w, 36w, and after treatment [41,53]. Both 23w and 36w mice shown in the main figures had confirmed proteinuria. Some NZB/W mice with proteinuria (>300 mg/dl) at two timepoints were treated with combination therapy—one dose of 50 mg/kg CTX, six doses of 100 μg CTLA-4-Ig, and one dose 250 μg of anti-CD40L41. Mice were determined to be in remission if they achieved proteinuria of ≤30 mg/dl at two timepoints [41]. One group was sacrificed 3-4 weeks after remission (Tx Rem. +3-4w) and another was sacrificed>5-14 weeks after remission (Tx Rem.+>5w) [41]. The latter group had confirmed histologic relapse [41]. Kidney gene expression from NZW/BXSB mice was measured at 17w (prenephritic mice) or 18-21w mice with confirmed proteinuria [53].

GSE72410 comprises samples from the IFNα-accelerated NZB/W LN model. NZB/W mice were treated with adenovirus-expressing murine IFNα at 14-15w (1.2×108 IFNα rAd5-CMV)64. Kidney gene expression was measured in 17w naïve NZB/W (naïve), as well as IFNα-accelerated NZB/W mice: 17w mice 21 days post IFNα injection (IFN W3), and in mice treated with vehicle or CTX for four weeks beginning three weeks post IFNα injection (IFN W7+Veh or IFN W7+CTX) [64].

GSE153021 comprises samples from the MRL/lpr LN model. MRL lpr mice were treated with vehicle, prednisone, mycophenolate mofetil (MMF), FK506, or all three (Multi-target) for eight weeks beginning at week 8 or age-matched wildtype MRL/MpJ mice treated with vehicle [65].

Quality control and data normalization were performed as follows. Microarray data were processed [66] using free, open source programs (GEOquery, affy, affycoretools, limma, and simpleaffy). Unnormalized arrays were inspected for visual artifacts or poor RNA hybridization using QC plots. Datasets were annotated using their native chip definition files (CDFs). Probes missing gene annotation data were discarded. Raw data (CEL files) from the Affymetrix platform were background corrected and normalized using guanine cytosine robust multiarray average (GCRMA) or robust multichip average (RMA) algorithms, whereas raw data files from Illumina chip were read and normalized using neqc (limma R package).

RNA-seq data (GSE72410 and GSE153021) was processed from FASTQ files as described by Daamen [67] and also described below. SRA files were downloaded and converted into FASTQ format. Read ends and adapters were trimmed with Trimmomatic (v0.38) using a sliding window, ilmnclip, and headcrop filters. The reads were head cropped at 6 bp and adapters were removed before read alignment. Reads were mapped to the mouse reference genome m17 using STAR, and the .sam files were converted to sorted .bam files using Sambamba. The mouse reference genome was downloaded from GENCODE. Read counts were summarized using the featureCounts function of the Subread package (v1.61). The DESeq2 workflow was used to filter RNA-seq genes with low expression (i.e. genes with very few reads). The filtered raw counts were normalized using DESeq method and then log 2 transformed.

Principal component analysis was used to inspect the raw data files from each dataset for outliers. All log 2 transformed data was formatted into R expression set objects (E-sets).

DEG analysis was performed as follows. For human dataset DEG analysis, Affymetrix probes were additionally annotated with custom BrainArray (BA) chip definition files (CDFs) [66, 68]. Any probes with different Affymetrix and BA gene annotations were excluded. GCRMA-normalized expression values were variance corrected using local empirical Bayesian shrinkage before calculation of DEGs using the ebayes function in the BioConductor LIMMA package. P-values were adjusted for multiple hypothesis testing using the Benjamini-Hochberg correction, which resulted in a False Discovery Rate (FDR). Significant Affymetrix and BA probes within each study were merged and filtered to retain probes with a pre-set FDR<0.2 which were considered statistically significant. This FDR was employed to avoid falsely excluding genes of interest. This list was further filtered to retain only the most significant probe per gene in order to remove duplicate genes.

Network analysis and visualization were performed as follows. Cytoscape (V3.8.0) [69] with the Search Tool for the Retrieval of Interacting Genes/Proteins (STRING) (V1.5.1) and ClusterMaker2 (V1.3.1) [70] plugins was used to create and visualize protein-protein interactions among the 883 common DEGs. Clusters were generated with the Molecular Complex Detection (MCODE) clustering algorithm within ClusterMaker2 and a node score cutoff of 0.2, k-Core of 2, and a max depth of 100 were set.

Functional Enrichment Analysis was performed as follows. Functional enrichment of Cytoscape-derived MCODE clusters was performed using BIG-C, a clustering tool developed to categorize the biologic function of large lists of genes [66]. The top three significant BIG-C categories (p<0.05, OR>1) were reported.

Gene Set Variation Analysis (GSVA) was performed as follows. GSVA22 for R/Bioconductor was used as a non-parametric, unsupervised method for estimating the variation of pre-defined gene sets in dataset samples. For each dataset only one CDF, Affymetrix or Illumina, was used for each probe. For genes with multiple Affymetrix probe identifiers, only the probe with the highest inter-quartile range (IQR) of expression [71] was retained. Genes with IQR=0 were removed. GSVA enrichment scores were calculated non-parametrically using a Kolmogorov Smirnoff (KS)-like rank statistic [22]; a negative value for a particular sample and gene set means that the gene set has a lower expression than the gene set with a positive value. The same GSVA probes used in calculations for class III/IV LN GL and TI were specified for calculations in all classes of LN.

GSVA Gene Sets were analyzed as follows. Metabolism gene sets were created from literature mining. Hematopoietic gene sets were derived from Immune/Inflammatory-Scope (I-Scope), a tool developed to identify immune cell-specific genes in big data [72]. Non-hematopoietic cellular gene sets were derived from T-Scope [72] or literature mining. Many gene sets in T-Scope were derived from The Human Protein Atlas (www.proteinatlas.org) [73,74]. The keratinocyte signature was derived from the keratinocyte-specific genes of Gazel et al. [75]. Kidney-specific lists generated from the Human Protein Atlas and single-cell data were additionally modified to incorporate genes found by both transcriptomics and immunohistochemistry [76]. The mesangial cell signature was derived from PanglaoDB [77]. In all tissues, the hematopoietic, EC, and fibroblast gene sets were evaluated. For non-hematopoietic gene signatures, those relevant to each tissue were reported (DLE—keratinocyte, melanocyte; LN GL—kidney cell, podocyte, mesangial cell; LN TI-kidney cell, proximal tubule, Loop of Henle (LoH) cell, distal tubule, collecting duct cell). Mitochondrial and peroxisomal gene sets were derived from literature mining and the BIG-C, and also compared to signatures in the MSIG database. The Apoptotic Mitochondrial Changes signature (M7482) was accessed on the MSIG database [78,79] and is derived from GO_0008637. GO_Mitochondrial_Fission (M12786) and GOBP_Peroxiome_Fission (M22828) signatures were accessed on the MSIG database and modified slightly. The IGS is the type I IFN core signature [35].

All human metabolism, non-hematopoietic cell, and IFN gene sets were converted to murine gene sets using the homologene R package and human2mouse function. Genes that were not converted programmatically were manually converted by GeneCards and Mouse Genome Informatics orthologs. Murine hematopoietic cell gene sets were curated by literature mining. For murine datasets with expression of two or more anergic/activated T cell markers, the anergic/activated T cell signature was combined with the T cell signature for GSVA analysis.

Although the same gene sets were input for each category in each tissue (Table 2), the genes used in calculation of the GSVA enrichment score for each tissue differ slightly based upon the gene measurement platform and expression within that sample. All reported GSVA enrichment scores, except for the HIF1A gene signature, were calculated based upon a minimum of three genes. The HIF1A gene signature only includes HIF1A because there were no additional genes determined to be specific to hypoxia only. Although two genes were well co-expressed, the DC signature in LN TI did not meet the minimum three gene requirement for GSVA, nor did the LDG signature in LN GL and LN TI.

Hierarchical clustering was performed as follows. Human lupus tissue samples were hierarchically clustered by the Euclidian distance of their GSVA enrichment scores into two (k=2) or four (k=4) clusters using the heatmap.2 function in R.

Regression Models were analyzed as follows. For all linear models, GSVA scores for cellular signatures in all tissue samples were input as independent variables, and the pathway GSVA score (metabolism signature, mitochondrial signature, or peroxisomal signature) was input as the dependent variable. As GSVA scales the expression of a signature from −1 to −1, the value for each input cellular or metabolic signature in each sample is relative to the same signature in other samples and to the other signatures in the same sample. To ensure that collinearity between immune cell signatures did not confound results for stepwise regression analyses in DLE, we combined the pDC, skin-specific DC, monocyte/MC, T cell, anergic/activated T cell, B cell, and plasma cell signatures into the “inflammatory cell” signature, because the genes were highly co-expressed. The list of all genes used as the “inflammatory cell” signature may be found in Table 2. This reduced the number of input variables for DLE stepwise analysis to ten cell signatures.

Visualization was performed as follows. Final figures were generated in GraphPad Prism or Adobe Illustrator.

Overlap p-values and ORs for functional enrichment of DEGs were calculated in R using two-sided fisher.test with confidence level=0.95. Because of known heterogeneity in lupus patients, the number of lupus patients in each tissue who fell above or below the control mean±1 standard deviation (SD) were then reported in order to determine whether individual patients exhibit an increased or decreased signature when the population did not achieve statistical significance. Calculation of mean and standard deviation (SD) for the control samples for each GSVA score in each tissue was performed in Microsoft Excel. All analyses in GraphPad Prism were carried out with version 8.2.0 (435) or later versions. Control and lupus sample populations of GSVA scores for each gene set were assessed for normality using the D'Agostino-Pearson test in GraphPad Prism, and the distributions for 75% or more of the gene sets in each population were determined to be normal. Welch's t-test with Bonferroni correction for GSVA enrichment in human samples was performed in GraphPad Prism. Bonferroni correction for metabolic signatures, immune cell signatures, non-hematopoietic signatures, or mitochondrial/peroxisomal signatures were performed separately. The Mann-Whitney U test for GSVA enrichment in murine samples was performed in GraphPad Prism. Univariate (simple) linear regression and Pearson correlation analyses were carried out in GraphPad Prism. Hedges' g effect sizes were calculated in R using the cohen.d function with Hedges' correction under the “effsize” package. Stepwise regression was performed in R using the lm function followed by the step AIC function. Variance inflation factor (VIF)<10 was confirmed for each independent variable. Although some data points were determined to be influential to the stepwise equation using Cook's D, no samples were removed from the models in efforts to capture the heterogeneity present in lupus. CART analysis was performed in R using the rpart function with the “anova” method. Each resulting decision tree was pruned once except for the glycolysis and PPP signatures in LN TI. GSVA scores for all individual samples (patient or control) are presented as individual data points in either dot or violin plots. The number of samples for each group may be found in the figure legends. Information regarding the statistical comparisons made and level of significance is mentioned in the figure legends.

Data availability: All microarray datasets in this publication are available on NCBI's GEO database. All bioinformatic software used in this publication is open source, and freely available for R. Example codes used here (LIMMA, GSVA, stepwise regression, and CART) are available at figshare, www.figshare.com as “AMPEL BioSolutions LIMMA Differential Expression Analysis Code,” “AMPEL BioSolutions GSVA AFFY nonzeroIQR Code”, and “AMPEL BioSolutions Stepwise and CART Code,” respectively.

### Figure Legends

FIGS. 1A-1I show that dysregulation of metabolic gene signatures is common among lupus-affected tissues. FIG. 1A: Comparison of DEGs among DLE, class III/IV LN GL, and class III/IV LN TI. FIG. 1B: MCODE protein-protein interactions of common UP and DOWN DEGs were generated with Cytoscape using the STRING and ClusterMaker2 plugins and annotated with BIG-C functional categories (odds ratio (OR)>1, p<0.05) in Adobe Illustrator. Overlap p-value was calculated using Fisher's exact test. GSVA of signatures for glycolysis (FIG. 1C), the PPP (FIG. 1D), the TCA cycle (FIG. 1E), OXPHOS (FIG. 1F), FAAO (FIG. 1G), FABO (FIG. 1H), and AA metabolism in lupus tissues and controls (CTLs) (FIG. 1I). Each point represents an individual sample. Significant differences in enrichment of the metabolic signatures in each lupus tissue as compared to CTL was determined by Welch's t-test with Bonferroni correction. Numbers below each tissue indicate the number of lupus patients with enrichment scores 1 SD less than (<1SD) or greater than (>1SD) the CTL mean. For all calculations, the following sample numbers were used: DLE [CTL (n=8), DLE (n=9)], LN GL [CTL (n=14), Cl III/IV (n=22)], and LN TI [CTL (n=15), Cl III/IV (n=22)]. **, p<0.01; ***, p<0.001; ****, p<0.0001.

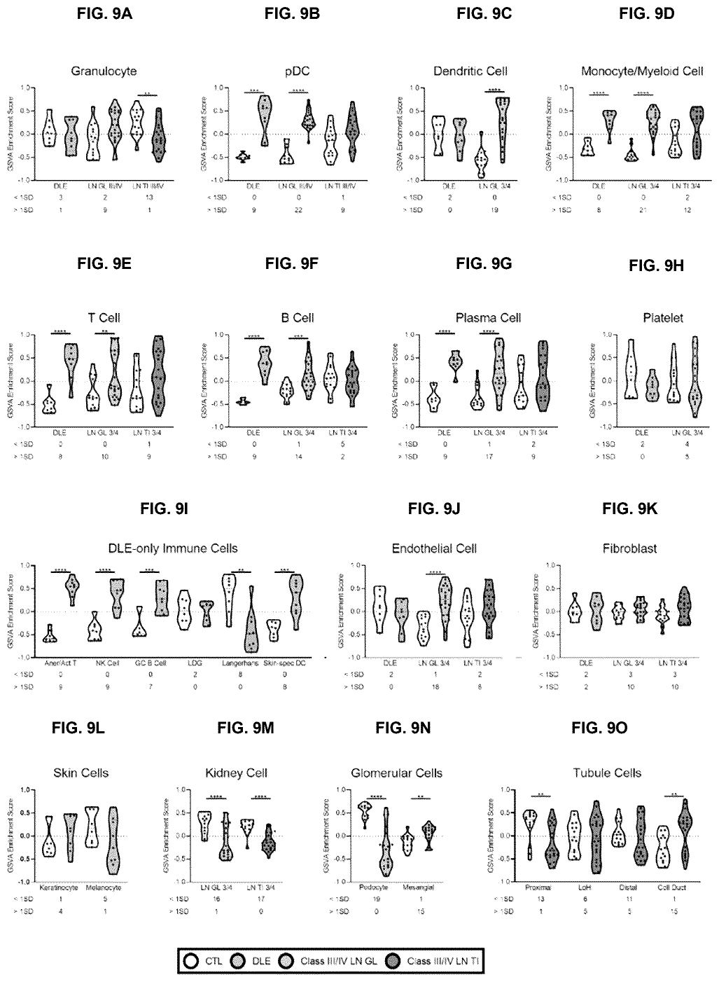

FIGS. 2A-2C show that increased myeloid cell signatures and decreased non-hematopoietic cell signatures characterize the majority of lupus patients. FIG. 2A: Hedges' g effect sizes of immune and non-hematopoietic cell signatures in DLE, class III/IV LN GL, and class III/IV LN TI as compared to tissue CTLs. Significant p-values reflect significant differences in enrichment of the immune cell signatures or non-hematopoietic cell signatures in lupus tissues as compared to CTL as determined by Welch's t-test with Bonferroni correction (FIG. 9). FIG. 2B: R² values derived from linear regression of the monocyte-derived macrophage or the tissue-resident macrophage markers with the monocyte/MC GSVA scores in individual patients and CTLs from lupus-affected tissues (FIGS. 11A-11B). Significant p-values reflect significantly non-zero slopes. FIG. 2C: Pearson correlation coefficients between tissue-resident macrophage markers in LN. For all calculations, the following sample numbers were used: DLE [CTL (n=8), DLE (n=9)], LN GL [CTL (n=14), Cl 3/4 (n=22)], and LN TI [CTL (n=15), Cl 3/4 (n=22)]. *, p<0.05; **, p<0.01; ***, p<0.001; ****, p<0.0001.

FIGS. 3A-3U show that metabolic and cellular signature changes in class II LN GL are similar to those seen in class III/IV. GSVA of metabolic pathway signatures (FIGS. 3A-3G) and cell signatures (FIGS. 3G-3T) in all classes of LN GL. Each point represents an individual sample. Significant differences in enrichment of the metabolic signatures, immune cell signatures, or non-hematopoietic cell signatures between class II LN GL and CTL, class III/IV LN GL and CTL, and class II LN GL and class III/IV LN GL were performed by Welch's t-test with Bonferroni correction. FIG. 3U: Hierarchical clustering (k=4) of all glomerular samples. For all calculations, the following sample numbers were used: LN GL [CTL (n=14), Cl II (n=8), Cl III/IV (n=22)]. **, p<0.01; ***, p<0.001; ****, p<0.0001.

FIGS. 4A-4H show that metabolic gene expression changes in LN GL are associated with changes in the EC, kidney cell, and fibroblast gene signatures. FIG. 4A: Stepwise regression coefficients and FIGS. 4B-4H CART analysis for metabolic pathway signatures in all glomerular LN samples and CTLs. Values in the final CART leaves for each process represent the average GSVA score of samples that were assigned to that leaf. Each resulting CART decision tree was pruned once. For all calculations, the following sample numbers were used: LN GL [CTL (n=14), Cl II (n=8), Cl III/IV (n=22)]. Significant p-values in FIG. 4A reflect significant coefficients in the stepwise regression model. *, p<0.05; **, p<0.01; ***, p<0.001; ****, p<0.0001.

FIGS. 5A-5O show that mitochondrial and peroxisomal signature changes and local hypoxia contribute to changes in metabolic gene expression in specific cells. GSVA of signatures for mitochondria- (FIGS. 5A-5F) or peroxisome-related gene signatures (FIGS. 5G-5H) in lupus tissues and CTLs. Each point represents an individual sample. FIGS. 5I-5K: Stepwise regression coefficients for mitochondrial and peroxisomal signatures in all tissues and CTLs. FIG. 5L: GSVA of HIF1A in lupus tissues and CTLs. Each point represents an individual sample. FIGS. 5M-5O: Stepwise regression coefficients for metabolic pathway signatures with the addition of HIF1A in all tissues and CTLs. Significant differences in enrichment of the mitochondrial/peroxisomal signatures or HIF1A in DLE and CTL, Cl II LN GL and CTL, Cl III/IV LN GL and CTL, Cl II LN TI and CTL, Cl III/IV LN TI and CTL was determined by Welch's t-test with Bonferroni correction. Numbers below each tissue indicate the number of lupus patients with enrichment scores 1 SD less than (<1SD) or greater than (>1SD) the CTL mean. For all calculations, the following sample numbers were used: DLE [CTL (n=8), DLE (n=9)], LN GL [CTL (n=14), Cl 2 (n=8), Cl III/IV (n=22)], and LN TI [CTL (n=15), Cl II (n=8), Cl III/IV (n=22)]. Significant p-values in FIGS. 5I-5K and FIGS. 5M-5O reflect significant coefficients in the stepwise regression model. *, p<0.05; **, p<0.01; ***, p<0.001; ****, p<0.0001.

FIGS. 6A-6H show that metabolic gene expression changes occur independent of acute IFN stimulation in murine LN. FIG. 6A: GSVA of the IGS in the kidney of IFNα-accelerated NZB/W mice (GSE86423). FIGS. 6B-6H: GSVA of metabolic signatures and linear regression between the IGS and metabolic signature GSVA scores. Each point represents an individual mouse. For each signature, significant differences in enrichment at each timepoint compared to baseline were evaluated with the Mann-Whitney U test. For all calculations the following sample numbers were used: Baseline (n=3), W1 (n=5), W2 (n=5), W3 (n=5), W5 (n=5), W7 (n=5), and W9 (n=5). Significant p-values in the regression plots in FIGS. 6B-6H reflect significantly non-zero slopes. *, p<0.05; **, p<0.01; ***, p<0.001; ****, p<0.0001.

FIGS. 7A-7E show that metabolic gene expression changes in murine LN are corrected with immunosuppressive treatment. GSVA of metabolism signatures in the kidney of NZM2410 (GSE32583, GSE49898) (FIG. 7A), NZB/W (GSE32583, GSE49898) (FIG. 7B), IFNα-accelerated NZB/W (GSE72410) (FIG. 7C), MRL/lpr (GSE153021) (FIG. 7D), and NZW/BXSB (GSE32583, GSE49898) mice (FIG. 7E) with and without treatment. Each point represents an individual mouse. For each signature, significant differences in enrichment at each timepoint compared to baseline and each treatment timepoint compared to disease were evaluated with the Mann-Whitney U test. For all calculations the following sample numbers were used: NZM2410 [6w (n=5), 21-30w (n=5), Tx+15w (n=6)], NZB/W [16w (n=8), 23w (n=6), 36w (n=10), Tx Remission+3-4w (n=8), Tx Remission+>5w (n=6)], IFN-accelerated NZB/W [Naïve (n=5), IFN W3 (n=5), IFN W7+Veh (n=5), IFN W7+CTX (n=5)], MRL/lpr [Wildtype (n=3), Vehicle (n=3), Prednisone (n=3), MMF (n=3), FK506 (n=3), Multi-target (n=3)], and NZW/BXSB [17w (n=6), 18-21w+P (n=6)]. *, p<0.05; **, p<0.01; ***, p<0.001; ****, p<0.0001.

FIGS. 8A-8F show that cellular and metabolic gene expression changes correlate with expression of genes indicating tubular damage in human and murine LN. Log2 expression of HAVCR1 Havcr1 (FIG. 8A) and LCN2 Lcn2 (FIG. 8B) in human LN TI and the kidneys of (NZM2410 (GSE32583, GSE49898), NZB/W (GSE32583, GSE49898), IFNα-accelerated NZB/W (GSE86423), IFNα-accelerated NZB/W (GSE72410), and MRL/lpr (GSE153021) mice. Each point represents an individual human or mouse. Significant differences in gene expression at each timepoint compared to disease were evaluated in the human TI with Welch's t-test and in murine models with the Mann-Whitney U test. Linear regression between HAVCR1 Havcr1 and LCN2 Lcn2 expression and kidney cell, proximal tubule, and TCA cycle GSVA scores in all samples for human (FIGS. 8C-8E) and murine (FIG. 8F) LN. For all calculations the following sample numbers were used: LN TI [CTL (n=15), Cl II (n=8), Cl III/IV (n=22)], NZM2410 [6w (n=5), 21-30w (n=5), Tx+15w (n=6)], NZB/W [16w (n=8), 23w (n=6), 36w (n=10), Tx Remission+3-4w (n=8), Tx Remission+>5w (n=6)], IFN-accelerated NZB/W (GSE86423) [Baseline (n=3), W1 (n=5), W2 (n=5), W3 (n=5), W5 (n=5), W7 (n=5), and W9 (n=5), IFN-accelerated NZB/W (GSE72410) [Naïve (n=5), IFN W3 (n=5), IFN W7+Veh (n=5), IFN W7+CTX (n=5)], and MRL/lpr [Wildtype (n=3), Vehicle (n=3), Prednisone (n=3), MMF (n=3), FK506 (n=3), Multi-target (n=3)]. Significant p-values in (c-f) reflect significantly non-zero slopes determined by linear regression or correlation analysis. *, p<0.05; **, p<0.01; ***, p<0.001; ****, p<0.0001.

FIGS. 9A-9O show that increased myeloid cell signatures and decreased non-hematopoietic cell signatures characterize the majority of lupus patients. GSVA of signatures for granulocytes (FIG. 9A), pDCs (FIG. 9B), dendritic cells (FIG. 9C), monocyte/MCs (FIG. 9D), T cells (FIG. 9E), B cells (FIG. 9F), plasma cells (FIG. 9G), platelets (FIG. 9H), immune cells (FIG. 91) with expression found only in DLE, endothelial cells (FIG. 9J), fibroblasts (FIG. 9K), skin cells (FIG. 9L), kidney cells (FIG. 9M), glomerular cells (FIG. 9N), and tubule cells (FIG. 9O) in lupus tissues and CTLs. Each point represents an individual sample. Significant differences in enrichment of the immune cell signatures or non-hematopoietic cell signatures in each lupus tissue as compared to CTL was determined by Welch's t-test with Bonferroni correction. Numbers below each tissue indicate the number of lupus patients with enrichment scores 1 SD less than (<1SD) or greater than (>1SD) the CTL mean. For all calculations, the following sample numbers were used: DLE [CTL (n=8), DLE (n=9)], LN GL [CTL (n=14), Cl III/IV (n=22)], and LN TI [CTL (n=15), Cl III/IV (n=22)]. **, p<0.01; ***, p<0.001; ****, p<0.0001.

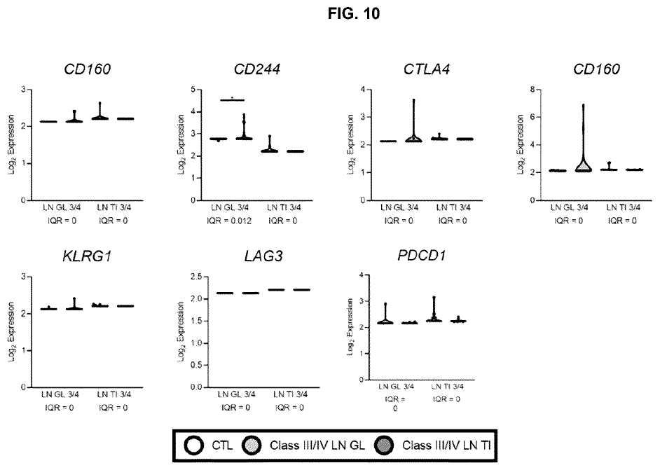

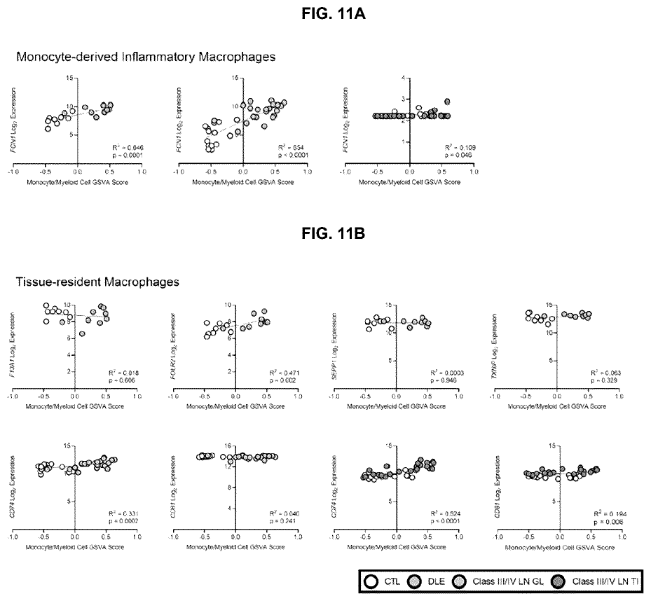

FIG. 10 shows that anergic/activated T cell marker genes have no change in expression in LN class III/IV. Log2 expression of CD160, CD244, CTLA4, ICOS, KLRG1, LAG3, and PDCD1 in lupus tissues and CTLs. Each point represents an individual sample. Significant differences in expression of the anergic/activated T cell marker gene in each lupus tissue as compared to CTL were determined by Welch's t-test. Value beneath the x-axis denotes the interquartile range (IQR) of expression calculated in R. For all calculations, the following sample numbers were used: LN GL [CTL (n=14), Cl III/IV (n=22)] and LN TI [CTL (n=15), Cl III/IV (n=22)]. *, p<0.05.

FIGS. 11A-11B show that monocyte/MC gene signatures reflect both monocyte-derived macrophage and tissue-resident macrophage populations. Linear regression between the monocyte/MC GSVA score and FCN1 expression (FIG. 11A) or TRM marker expression (FIG. 11B) in lupus-affected tissues. Each point represents an individual sample. For all calculations, the following sample numbers were used: DLE [CTL (n=8), DLE (n=9)], LN GL [CTL (n=14), Cl III/IV (n=22)], and LN TI [CTL (n=15), Cl III/IV (n=22)]. Significant p-values reflect significantly non-zero slopes.

FIG. 12 shows that metabolic and cellular gene expression changes in class II LN GL are similar to those seen in class III/IV. Hierarchical clustering (k=4) of class II LN GL samples (n=8). Red stars represent patients with a GSVA score greater than the control mean+1 SD. Black stars represent patients with a GSVA score less than the control mean −1SD.

FIGS. 13A-13U show that metabolic and cellular gene expression changes in class II LN TI are less robust than those seen in class III/IV. GSVA of metabolic pathway signatures (FIGS. 13A-13G) and cell signatures (FIGS. 13H-13T) in all classes of LN TI. Each point represents an individual sample. Significant differences in enrichment of the metabolic signatures, immune cell signatures, or non-hematopoietic cell signatures between class II LN TI and CTL, class III/IV LN TI and CTL, and class II LN TI and class III/IV LN TI were performed by Welch's t-test with Bonferroni correction. FIG. 13U: Hierarchical clustering (k=4) of all tubulointerstitial samples. For all calculations, the following sample numbers were used: LN TI [CTL (n=15), Cl II (n=8), Cl III/IV (n=22)]. **, p<0.01; ***, p<0.001; ****, p<0.0001.

FIG. 14 shows that metabolic and cellular gene expression changes in some class II LN TI patients are similar to those seen in class III/IV patients. Hierarchical clustering (k=4) of class II LN TI samples (n=8). Red stars represent patients with a GSVA score greater than the control mean+1 SD. Black stars represent patients with a GSVA score less than the control mean−1SD.

FIGS. 15A-15B show that numerous cellular gene signatures contribute to the observed metabolic changes in DLE. FIG. 16A: Stepwise regression coefficients for metabolic pathway GSVA scores in all samples for DLE and CTLs. For stepwise repression the pDC, skin-specific DC, monocyte/MC, T Cell, anergic/activated T cell, B cell, and plasma cell signatures were combined into the “inflammatory cell” signature because of collinearity. FIG. 16B: Hierarchical clustering (k=2) of all skin samples. For all calculations, the following sample numbers were used: DLE [CTL (n=8), DLE (n=9)]. Significant p-values reflect significant coefficients in the stepwise regression model. *, p<0.05; **, p<0.01; ***, p<0.001; ****, p<0.0001.

FIGS. 16A-16H show that metabolic gene expression changes in LN TI are associated with changes in the kidney cell, proximal tubule, and monocyte/MC gene signatures. Stepwise regression coefficients (FIG. 16A) and CART (FIG. 16B-16H) analysis for metabolic pathway signatures in all tubulointerstitial LN samples and CTLs. Values in the final CART leaves for each process represent the average GSVA score of samples that were assigned to that leaf. Each resulting CART decision tree except for glycolysis and PPP was pruned once. For all calculations, the following sample numbers were used: LN TI [CTL (n=15), Cl II (n=8), Cl III/IV (n=22)]. Significant p-values in FIG. 16A reflect significant coefficients in the stepwise regression model. *, p<0.05; **, p<0.01; ***, p<0.001; ****, p<0.0001.

FIG. 17 shows that metabolic genes are altered in scRNA-seq from LN biopsies. DEGs related to metabolism in scRNA-seq clusters (CM2: tissue-resident macrophages, CT0a: effector memory CD4+ T cells, and CEO: epithelial cells) that were present in both LN patients and CTL samples from Arazi et al (Ref. 30). Reported metabolic genes are those with corrected p-values<0.05.

FIGS. 18A-18Q show that cellular gene expression changes in NZM2410 kidneys may be corrected with immunosuppressive treatment. GSVA of immune (FIGS. 18A-18H) and non-hematopoietic (FIGS. 18I-18Q) cell signatures in the kidneys of NZM2410 mice (GSE32583, GSE49898) with and without treatment. Each point represents an individual mouse. For each signature significant differences in enrichment at each timepoint compared to baseline and treatment compared to disease were evaluated with the Mann-Whitney U test. For all calculations the following sample numbers were used: 6w (n=5), 21-30w (n=5), and Tx+15w (n=6). *, p<0.05; **, p<0.01; ***, p<0.001; ****, p<0.0001.

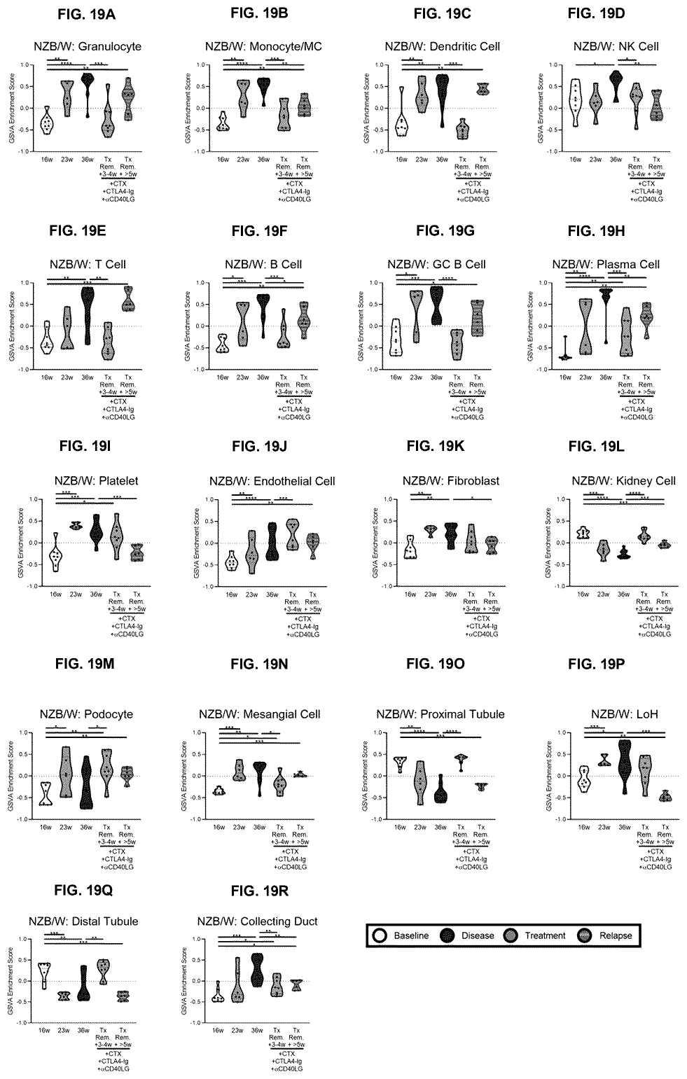

FIGS. 19A-19R show that cellular gene expression changes in NZB/W kidneys may be corrected with immunosuppressive treatment. GSVA of immune (FIGS. 19A-19I) and non-hematopoietic (FIGS. 19J-19R) cell signatures in the kidneys of NZB/W mice (GSE32583, GSE49898) with and without treatment. Each point represents an individual mouse. For each signature significant differences in enrichment at each timepoint compared to baseline and treatment compared to disease were evaluated with the Mann-Whitney U test. For all calculations the following sample numbers were used: 16w (n=8), 23w (n=6), 36w (n=10), Tx Remission+3-4w (n=8), and Tx Remission+>5w (n=6). *, p<0.05; **, p<0.01; ***, p<0.001; ****, p<0.0001.

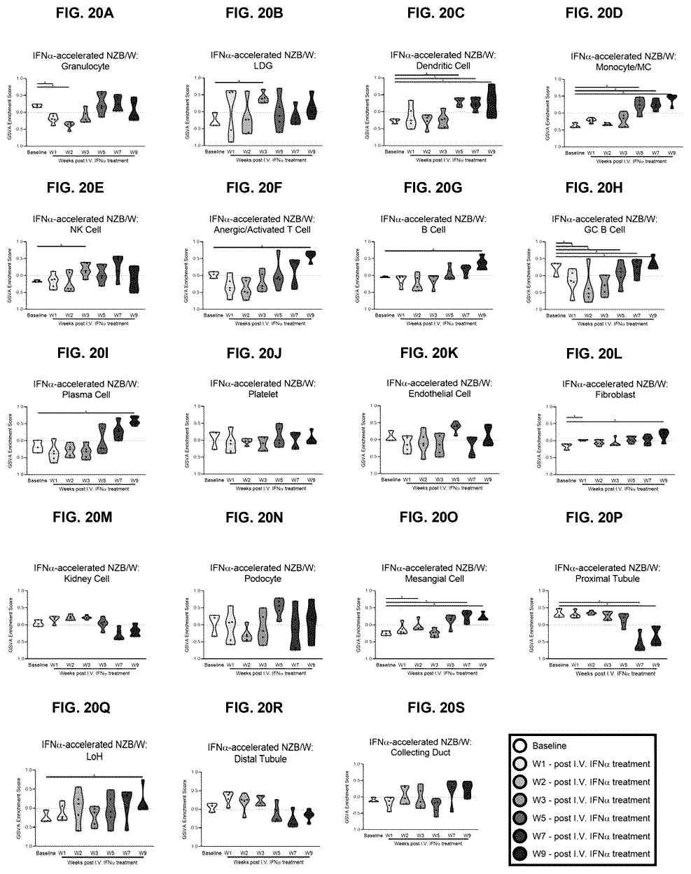

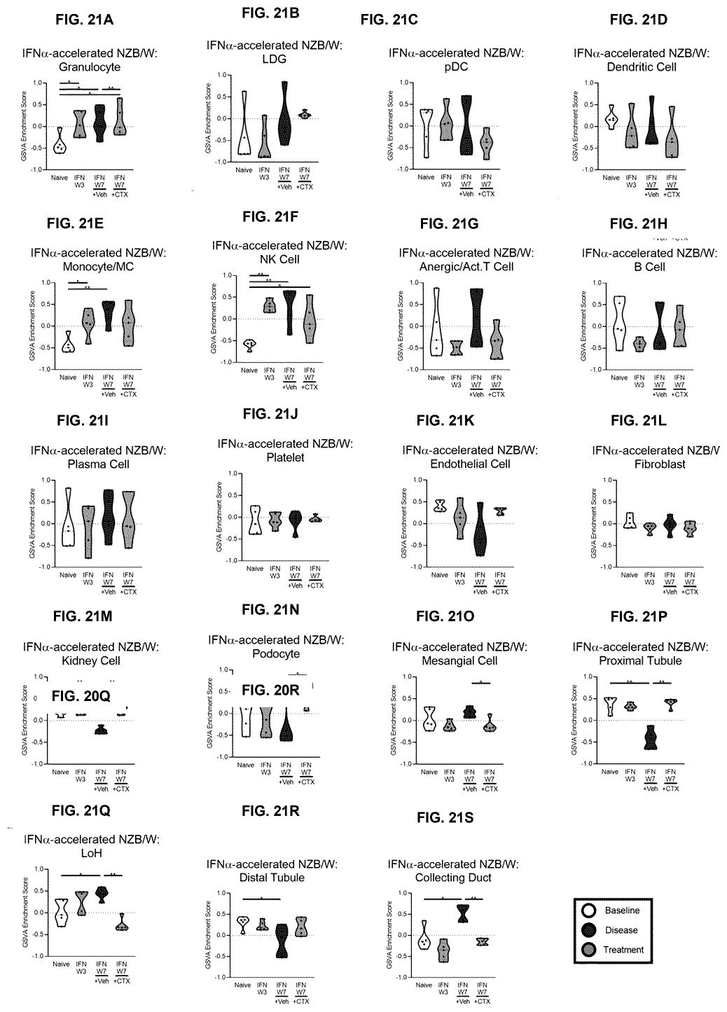

FIGS. 20A-20S show that immune/inflammatory cell gene expression is increased and proximal tubule cell gene expression is decreased in IFNα-accelerated NZB/W kidneys. GSVA of immune (FIGS. 20A-20J) and non-hematopoietic (FIGS. 20K-20S) cell signatures in the kidneys of IFNα-accelerated NZB/W mice (GSE86423). Each point represents an individual mouse. For each signature significant differences in enrichment at each timepoint compared to baseline were evaluated with the Mann-Whitney U test. For all calculations the following sample numbers were used: Baseline (n=3), W1 (n=5), W2 (n=5), W3 (n=5), W5 (n=5), W7 (n=5), and W9 (n=5). *, p<0.05; **, p<0.01; ***, p<0.001; ****, p<0.0001.

FIGS. 21A-21S show that cellular gene expression changes in IFNα-accelerated NZB/W kidneys may be corrected with immunosuppressive treatment. GSVA of immune (FIGS. 21A-21J) and non-hematopoietic (FIGS. 21K-21S) cell signatures in the kidneys of IFNα-accelerated NZB/W mice (GSE72410) with and without treatment. Each point represents an individual mouse. For each signature significant differences in enrichment at each timepoint compared to baseline and treatment compared to disease were evaluated with the Mann-Whitney U test. For all calculations the following sample numbers were used: Naïve (n=5), IFN W3 (n=5), IFN W7+Veh (n=5), and IFN W7+CTX (n=5). *, p<0.05; **, p<0.01; ***, p<0.001; ****, p<0.0001.

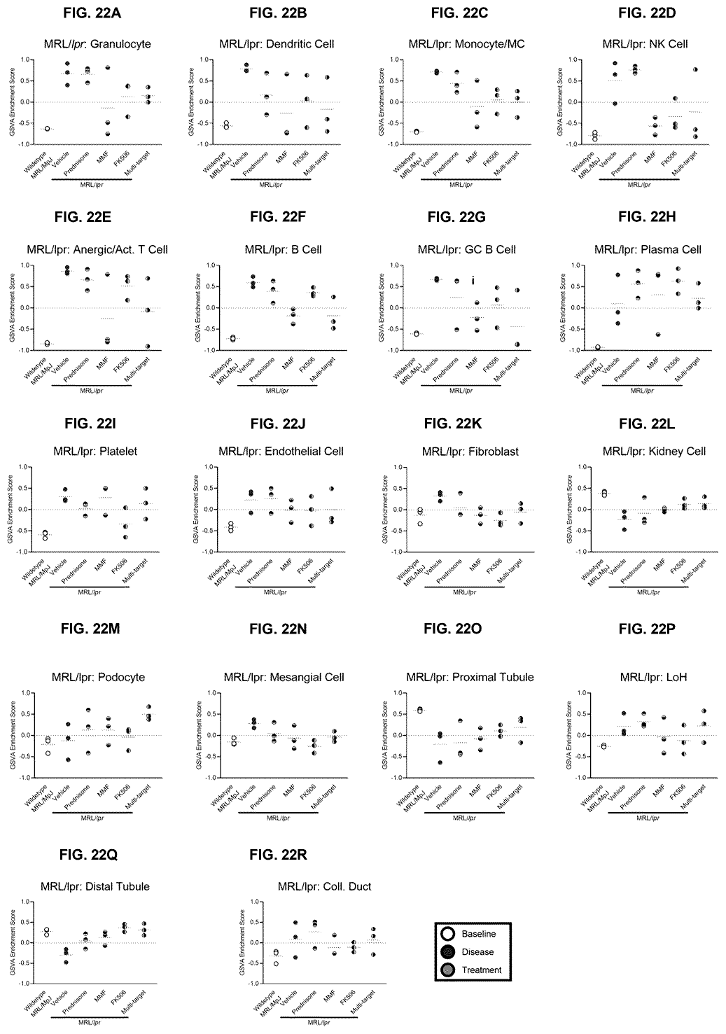

FIGS. 22A-22R show that cellular gene expression in the MRL/lpr kidney is not significantly altered. GSVA of immune (FIGS. 22A-22I) and non-hematopoietic (FIGS. 22J-22R) cell signatures in the kidneys of MRL/lpr mice (GSE153021) with and without treatment. Each point represents an individual mouse. For each signature significant differences in enrichment at each timepoint compared to baseline and each treatment timepoint compared to disease were evaluated with the Mann-Whitney U test. For all calculations the following sample numbers were used: Wildtype (n=3), Vehicle (n=3), Prednisone (n=3), MMF (n=3), FK506 (n=3), and Multi-target (n=3).

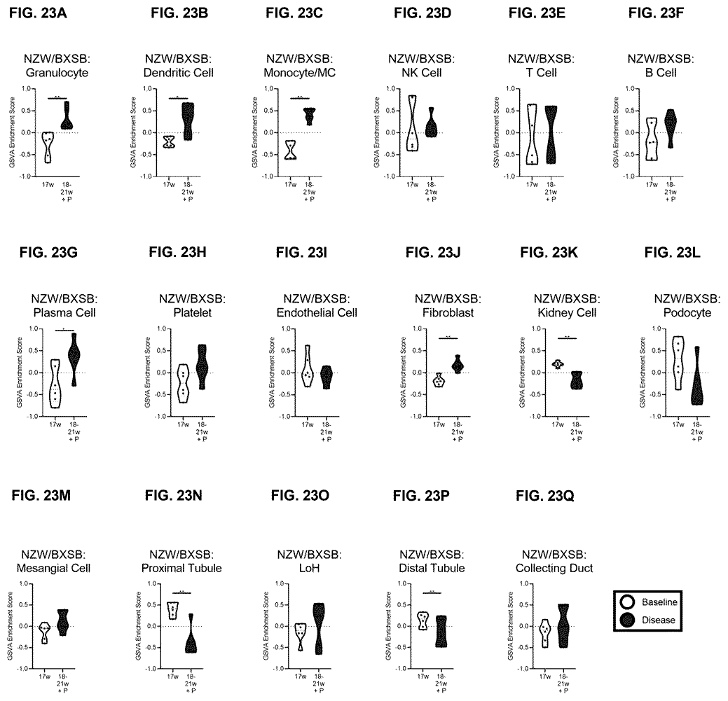

FIGS. 23A-23Q show that immune/inflammatory cell gene expression is increased and kidney cell and proximal tubule cell gene expression is decreased in NZW/BXSB kidneys. GSVA of immune (FIGS. 23A-23H) and non-hematopoietic (FIGS. 23I-23Q) cell signatures in the kidneys of NZW/BXSB mice (GSE32583, GSE49898). Each point represents an individual mouse. For each signature significant differences in enrichment at each timepoint compared to baseline were evaluated with the Mann-Whitney U test. For all calculations the following sample numbers were used: 17w (n=6) and 18-21w+P (n=6). *, p<0.05; **, p<0.01; ***, p<0.001; ****, p<0.0001.

FIGS. 24A-24F show that cellular gene expression changes in murine LN correlate with metabolic gene signatures. Pearson correlation coefficients for all metabolic pathway and cellular GSVA scores in all samples of each murine LN model NZM2410 (GSE32583, GSE49898) (FIG. 24A), NZB/W (GSE32583, GSE49898) (FIG. 24B), IFNα-accelerated NZB/W (GSE86423) (FIG. 24C), IFNα-accelerated (GSE72410) (FIG. 24D), MRL/lpr (GSE153021) (FIG. 24E), and NZW/BXSB (GSE32583, GSE49898) (FIG. 24F). For all calculations the following sample numbers were used: NZM2410 [6w (n=5), 21-30w (n=5), Tx+15w (n=6)], NZB/W [16w (n=8), 23w (n=6), 36w (n=10), Tx Remission+3-4w (n=8), Tx Remission+>5w (n=6)], IFN-accelerated NZB/W (GSE8642) [Baseline (n=3), W1 (n=5), W2 (n=5), W3 (n=5), W5 (n=5), W7 (n=5), and W9 (n=5)], IFN-accelerated NZB/W (GSE72410) [Naïve (n=5), IFN W3 (n=5), IFN W7+Veh (n=5), IFN W7+CTX (n=5)], MRL/lpr [Wildtype (n=3), Vehicle (n=3), Prednisone (n=3), MMF (n=3), FK506 (n=3), Multi-target (n=3)], and NZW/BXSB [17w (n=6), 18-21w+P (n=6)]. Significant p-values represent significantly non-zero slopes. *, p<0.05; **, p<0.01; ***, p<0.001; ****, p<0.0001.

FIG. 25 shows that cellular and metabolic gene expression changes correlate with expression of genes indicating tubular damage in murine LN. Correlation between Havcr1 or Lcn2 gene expression and GSVA scores for kidney cell, proximal tubule, and TCA cycle in all samples from the kidneys of NZM2410 (GSE32583, GSE49898), NZB/W (GSE32583, GSE49898), IFNα-accelerated NZB/W (GSE86423), IFNα-accelerated NZB/W (GSE72410), MRL/lpr (GSE153021), and NZW/BXSB (GSE32583) mice. For all calculations the following sample numbers were used: NZM2410 [6w (n=5), 21-30w (n=5), Tx+15w (n=6)], NZB/W [16w (n=8), 23w (n=6), 36w (n=10), Tx Remission+3-4w (n=8), Tx Remission+>5w (n=6)], IFN-accelerated NZB/W (GSE8642) [Baseline (n=3), W1 (n=5), W2 (n=5), W3 (n=5), W5 (n=5), W7 (n=5), and W9 (n=5)], IFN-accelerated NZB/W (GSE72410) [Naïve (n=5), IFN W3 (n=5), IFN W7+Veh (n=5), IFN W7+CTX (n=5)], MRL/lpr [Wildtype (n=3), Vehicle (n=3), Prednisone (n=3), MMF (n=3), FK506 (n=3), Multi-target (n=3)], and NZW/BXSB [17w (n=6), 18-21w+P (n=6)]. Significant p-values reflect significantly non-zero slopes.

FIGS. 26A-26F show alteration/dysregulation of metabolic gene signatures in lupus, psoriasis, atopic dermatitis, and scleroderma-affected tissues. Each graph shows comparison of DEGs among class III/IV LN GL (violin plot 2), class III/IV LN TI (violin plot 4), DLE (violin plot 6), PSO (violin plot 8), AD (violin plot 10), and SSc (violin plot 12), and respective controls (unshaded violin plots 1, 3, 5, 7, 9 and 11 in each panel). The graphs show GSVA of signatures for glycolysis (FIG. 26A), the PPP (FIG. 26B), the TCA cycle (FIG. 26C), OXPHOS (FIG. 26D), FABO (FIG. 26E), and AA metabolism in lupus tissues and controls (CTLs) (FIG. 26F). Each point represents an individual sample. Numbers below each tissue indicate the number of lupus patients with enrichment scores 1 SD less than (<1SD) or greater than (>1SD) the CTL mean. Significant p-values reflect significant differences in GSVA enrichment of the metabolic or cellular signatures in each lupus tissue as compared to CTL in was determined by Welch's t-test with Bonferroni correction. **, p<0.01; ***, p<0.001; ****, p<0.0001. See methods described in relation to FIGS. 1A-1I, Example 1.

FIGS. 27A and 27B show that increased immune cell signatures and decreased non-hematopoietic cell signatures characterize the majority of lupus patients. FIG. 27A: Hedges' g effect sizes of immune cell signatures in class III/IV LN GL, class III/IV LN TI, DLE, PSO, AD, and SSc as compared to tissue CTLs. FIG. 27B: Hedges' g effect sizes of non-hematopoietic cell signatures in class III/IV LN GL, class III/IV LN TI, DLE, PSO, AD, and SSc as compared to tissue CTLs. Significant p-values reflect significant differences in GSVA enrichment of the metabolic or cellular signatures in each lupus tissue as compared to CTL was determined by Welch's t-test with Bonferroni correction. **, p<0.01; ***, p<0.001; ****, p<0.0001. See methods described in relation to FIG. 2A, Example 1.

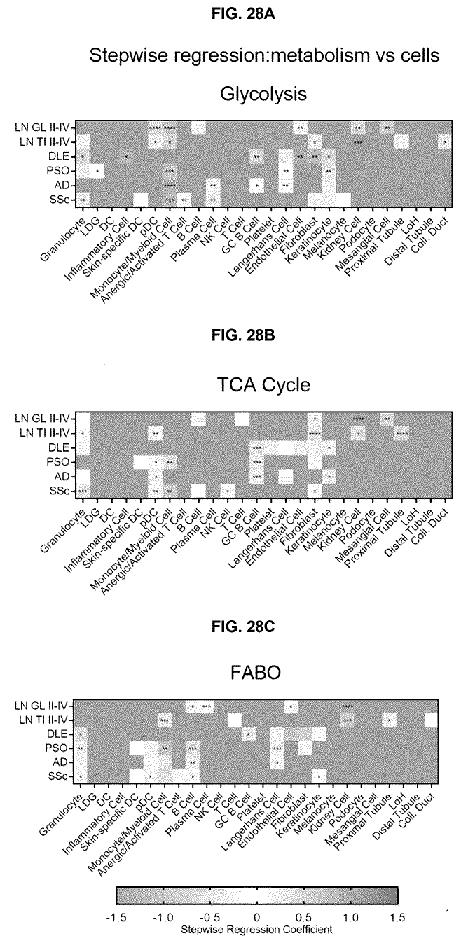

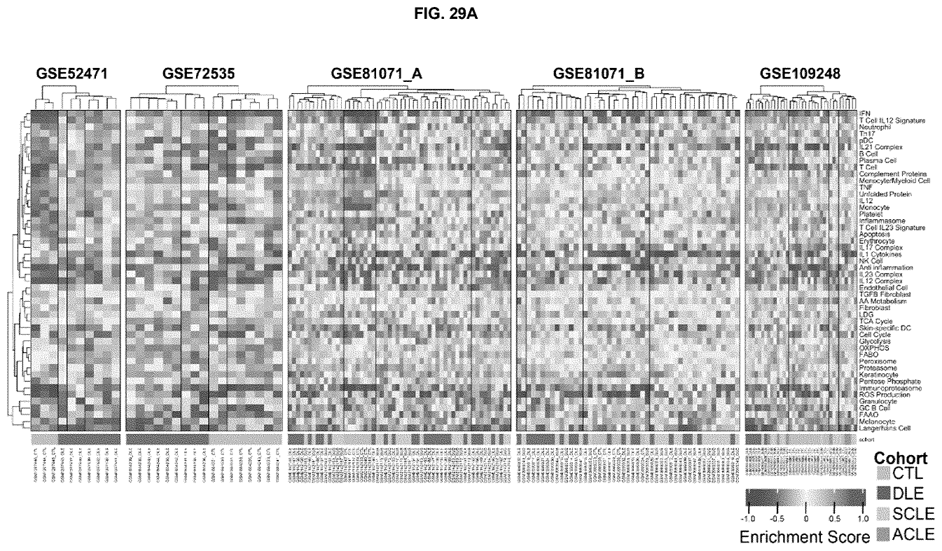

FIGS. 28A-28C show that metabolic and cellular gene signatures are concurrently altered in the tissues of inflammatory diseases, with different metabolic changes reflecting different cellular signatures. Stepwise regression coefficients are shown for the glycolysis (FIG. 28A), TCA cycle (FIG. 28B), and FABO (FIG. 28C) signatures in class II-IV LN GL, class II-IV LN TI, DLE, PSO, AD, SSc and tissue CTLs. Significant p-values reflect significant coefficients in the stepwise regression model. *, p<0.05; **, p<0.01; ***, p<0.001; ****, p<0.0001. See methods described in relation to FIGS. 15A and 16A, Example 1.

TABLE 1
883 common DEGs among human lupus tissues

AAMDC, AASS, ABCC10, ABCG1, ACAA1, ACAA2, ACACA, ACACB, ACAT1, ACO1,
ACOX2, ACSF2, ACTR3, ACVR1B, ADAMTS5, ADAP2, ADAR, ADCK2, ADCY1, ADII,
ADIPOR1, ADORA1, ADSL, AGPS, AHCY, AHCYL1, AIDA, AK4, AKAP10, AKAP7,
AKR1A1, AKR7A3, ALDH1A2, ALDH1L1, ALDH2, ALDH3A2, ALDH6A1, ALDH7A1,
ALOX5AP, AMMECR1, ANG, ANGEL2, ANGPT1, ANGPTL4, ANXA6, AOAH, APAF1,
APOBEC3B, APOBEC3G, APOE, APOL3, APOL6, ARG2, ARHGAP15, ARHGAP32,
ARHGDIB, ARHGEF6, ARID4B, ARIH1, ARL3, ARL4C, ARPC1B, ARPC2, ARPC5,
ARPP19, ARR3, ASS1, ATG12, ATP1B1, ATP5D, ATP5G3, ATP6V0E1, ATP9A, ATRN,
ATXN3, AXL, B2M, BAIAP2, BASP1, BAX, BBX, BCKDHA, BLVRA, BLVRB, BMP2K,
BNIP3L, BTN3A1, BTN3A2, BTN3A3, C11orf71, C14orf159, C17orf62, C19orf66,
C1GALT1C1, C1orf54, C1QA, C1QB, C2, C21orf33, C3, C3AR1, C3orf52, C5orf15, C6orf106,
C6orf62, CALCOCO2, CAMKK2, CAP1, CAPG, CARD8, CASP1, CASP3, CASP4, CASP7,
CASP8, CBR1, CBX1, CBX3, CCDC109B, CCDC51, CCL19, CCL5, CCL8, CCNB1,
CCNB1IP1, CCND2, CCR1, CCR5, CCT6B, CD14, CD163, CD2, CD3D, CD40, CD47, CD48,
CD52, CD53, CD74, CDC42EP4, CDK12, CDK13, CEBPD, CECR1, CELF2, CFAP45, CFB,
CHCHD2, CHI3L1, CHMP2A, CHMP5, CHP1, CHPT1, CIR1, CKAP2, CKLF, CKS2, CLIC1,
CLIP1, CLIP4, CMTR1, COL4A1, COL4A2, COL6A3, COMMD4, COQ10B, CORO1A,
COX4I1, COX7A1, CPVL, CRADD, CRAT, CRCP, CREB1, CRELD2, CRIP1, CRLF3, CS,
CSF1R, CSTA, CTGF, CTH, CTNNBIP1, CTSO, CTSS, CTSV, CX3CR1, CXCL10, CXCL12,
CXCR4, CYB5A, CYBB, CYC1, CYP4F2, CYP4F3, CYTIP, DACH1, DCK, DCLRE1C, DCP2,
DCTN3, DCUN1D2, DCXR, DDAH1, DDB1, DDB2, DDX28, DDX52, DDX58, DDX60,
DENND2D, DENND5A, DENR, DEXI, DFNA5, DGUOK, DHCR7, DHRS11, DHRS12,
DHRS3, DIO1, DLAT, DMXL2, DNAJA3, DNAJB1, DNAJC13, DNMT1, DOHH, DOLPP1,
DPM3, DRAM1, DSC2, DSE, DST, DYNLL1, DYNLT1, DYRK2, EBP, ECHDC2, ECI1, ECI2,
ECSIT, EEF2, EGF, EGR1, EIF2AK2, EIF2AK3, EIF4B, EIF4E2, EIF4G3, EIF5, ELF1, ELF4,
EMD, EMP3, ENG, ENSA, ENTPD1, EPHX1, EPM2A, ERO1L, ESRRA, ESRRG, ETF1, ETS2,
EVI2A, EVI2B, EXOSC10, FAAH, FABP5, FADD, FAM107A, FAM111A, FAM134B,
FAM49B, FAM89B, FAS, FASTK, FBXO21, FBX09, FCER1G, FCGR2A, FCHSD2, FDFT1,
FEN1, FER, FERMT2, FEZ2, FGF1, FGFR1, FGL2, FIBP, FKBP11, FKBP15, FKBP4, FLAD1,
FNBP1, FOLR2, FOSL2, FSCN1, FUCA1, FUT8, FYB, G0S2, GABARAPL1, GABRR2,
GALNT10, GARS, GART, GATM, GBP1, GBP2, GHITM, GHR, GIMAP4, GK, GLIPR1,
GMFG, GOLGA2, GOLM1, GOT1, GPAA1, GPD1, GPD1L, GPX7, GRB2, GRN, GRPEL1,
GSS, GSTA3, GSTM2, GUCY1A3, GUSBP11, GYPC, GZMA, GZMK, H2AFY, H2AFY2,
HADH, HADHA, HAUS3, HCK, HCLS1, HCP5, HDAC1, HDHD1, HEG1, HERC5, HERC6,
HGD, HIST1H2AC, HIST1H2BD, HIST1H2BK, HIVEP2, HLA-A, HLA-B, HLA-DMA, HLA-
DMB, HLA-DPA1, HLA-DPB1, HLA-DRA, HLA-DRB6, HLA-E, HLA-F, HLA-G, HMGCR,
HMGCS2, HMHA1, HMOX2, HN1, HNRNPDL, HOXB2, HOXB7, HOXC6, HOXD1, HRSP12,
HSD17B8, HTRA2, HYAL1, HYAL2, ICAM2, ID3, IDH3B, IDI1, IER3, IFI16, IFI27, IFI30,
IFI35, IFI44, IFI44L, IFI6, IFIH1, IFIT1, IFIT2, IFIT3, IFIT5, IFITM1, IFITM2, IFITM3,
IFNAR1, IGFBP7, IGJ, IK, IKBKB, IKZF5, IL15, IL18, INVS, IQCC, IRF7, IRF8, IRF9, IRS2,
ISG15, ISG20, ISOC2, ITGB1, ITGB2, ITM2C, ITPA, JADE1, KBTBD11, KCTD12, KDELC1,
KDELR3, KDM6B, KHNYN, KIAA0226, KIAA1551, KIF2A, KLF9, KLHDC2, KLHDC3,
LAP3, LAPTM5, LARS2, LASP1, LCP1, LCP2, LDLRAP1, LEPROTL1, LETM1, LGALS3BP,
LGALS9, LHFPL2, LIN37, LINC00623, LIPA, LIPC, LMAN1, LONRF3, LPAR6, LPXN,
LRRFIP2, LST1, LTB, LUZP1, LY6E, LY75, LY86, LY96, LYN, LYZ, M6PR, MACROD1,
MAD2L1BP, MAFF, MAGT1, MAL, MALL, MAML1, MAN2A2, MAN2B1, MAOA,
MAPK14, MARC2, MARCH6, MARCKS, MARCKSL1, MARS, MAT2A, MBNL2, MCCC2,
MCM2, MDK, MED1, MED24, MED6, MEF2C, METTL13, MFN1, MFSD1, MGEA5,
MKRN1, MNDA, MOB1A, MOCS1, MOK, MPC1, MPRIP, MR1, MRPL17, MS4A4A,
MS4A6A, MSRA, MT1X, MTFR1, MVD, MVK, MX1, MX2, MXI1, MYCBP2, MYD88,
N4BP2L2, N6AMT1, NAB2, NADK, NAGA, NAGK, NAGPA, NAT9, NCBP1, NDNF,
NECAP2, NEDD4L, NEDD9, NENF, NEU1, NFIL3, NGLY1, NID1, NKIRAS2, NKTR, NME6,
NMI, NOL3, NOL7, NPAT, NPC2, NQO2, NR2C1, NR2F2, NR2F6, NR3C1, NREP, NRG1,
NSL1, NT5E, NUBP1, NUDT9, NUP43, NUP62, NUP85, NUP93, NUPL2, OAS1, OAS2,
OAS3, OASL, OFD1, OGT, OLFML3, OPA1, OPHN1, OPLAH, PAK4, PAM16, PANK2,
PARP12, PARP8, PC, PCBP2, PCDHB12, PCNA, PDE8A, PDIA4, PDK4, PDZD8, PDZK1,
PEBP1, PECAM1, PEG3, PEX16, PEX19, PEX5, PEX7, PGAP3, PGM1, PHB, PHF11,
PHF21A, PHKA2, PHYH, PI4K2A, PIAS2, PICALM, PIGH, PIK3CD, PILRA, PINK1,
PIP4K2A, PLA1A, PLBD1, PLEKHA5, PLSCR1, PMEPA1, PMM1, POLR2C, PPIC, PPP2CB,
PPP2R3C, PPP2R5C, PPT1, PQBP1, PQLC1, PRCP, PRKCDBP, PRKCH, PRKD2, PROCR,
PROSC, PRPF3, PRPF40A, PRPS1, PRSS22, PRX, PSEN2, PSMB8, PSMB9, PSME1, PSME2,
PTGER3, PTPN2, PTPRC, PTPRN2, PTTG1, PXMP2, PYCARD, RAB31, RAB32, RAB40B,
RAB8B, RAB9A, RABIF, RAC2, RAP2C, RARRES3, RASGRP3, RASSF1, RB1, RBCK1,
RBM4, RBM5, RBPJ, RBX1, RC3H2, RCAN1, RCBTB2, RECQL, REEP2, RFC4, RFTN1,
RFX5, RHEB, RHOBTB3, RHOG, RIPK4, RMI1, RNASE1, RNASE6, RNASET2, RNF130,
RNF44, RNPEP, RPL14, RPL15, RPS28, RPS6KC1, RRBP1, RRM2, RSAD2, RTP4, S100A10,
S100A11, S100A4, SAMD9, SAMHD1, SAMSN1, SAP18, SATB1, SCARB2, SCPEP1, SDC1,
SDHC, SEL1L3, SERGEF, SERPINF1, SERPING1, SERPINH1, SERPINI1, SETD1B, SETD3,
SETDB1, SF3B3, SGK1, SH2B3, SH3BGRL3, SH3D21, SH3YL1, SHC1, SHMT1, SIK2,
SIRT1, SKP2, SLA, SLC22A5, SLC23A2, SLC25A11, SLC25A15, SLC25A17, SLC25A23,
SLC25A3, SLC25A37, SLC35F6, SLC38A1, SLC39A14, SLC39A7, SLC5A12, SLCO2B1,
SMARCA5, SMCHD1, SNRNP70, SNX10, SNX2, SNX6, SOCS3, SORD, SOWAHC, SP100,
SP110, SPATS2L, SPG11, SPG7, SPP1, SPRY2, SPTBN4, SPTLC2, SRBD1, SRSF6, SRSF8,
SS18, ST13, ST6GAL1, STAT1, STOML2, STRAP, STX12, SUCLG1, SUCLG2, SURF1, SYK,
SYNCRIP, SYNJ1, SYNRG, SZRD1, TACC2, TAF15, TAGLN, TAOK3, TAP1, TAPBP,
TBCB, TBCC, TBK1, TCAP, TCF4, TDRD7, TFPI2, TGFBR2, TGFBR3, THEM6, TIMP1,
TIMP3, TLDC1, TLK2, TLR2, TLR3, TMBIM6, TMEM140, TMEM194A, TMEM204,
TMEM41B, TMEM50A, TMEM8A, TMSB10, TNFRSF14, TNFSF10, TNNC1, TNPO2, TNS2,
TOB1, TOM1L1, TOR1A, TPD52, TPK1, TPP1, TPR, TPT1, TRAFD1, TRAPPC11, TRAPPC2,
TRAPPC6A, TREX1, TRIM14, TRIM21, TRIM22, TRIM27, TRIM38, TRIM5, TRIOBP,
TRIP6, TSSC1, TYROBP, UBD, UBE2C, UBE2D2, UBE2J1, UBE2L6, UBFD1, UBN1, UBP1,
UBXN7, UCP2, UPB1, UPF2, UQCRC1, UQCRFS1, USP18, USP24, USP48, VCAM1, VEGFA,
VIPAS39, VOPP1, VPS11, VRK2, WARS, WASL, WDR13, WIPF1, XAF1, XPNPEP1, XPO6,
YARS, YEATS2, YWHAB, YWHAE, YWHAH, YWHAZ, ZBTB16, ZBTB24, ZBTB40,
ZC3HAV1, ZCCHC2, ZDHHC14, ZDHHC7, ZFAND5, ZFYVE26, ZKSCAN4, ZMYM6,
ZMYM6NB, ZNF200, ZNF219, ZNF224, ZNF264, ZNF331, ZNF350, ZNF45, ZNF562,
ZNF623, ZNF91

TABLE 2
GSVA gene sets of human or mouse genes

Amino Acid Metabolism (Human): AASS, AGXT, AGXT2, ALDH4A1, ALDH7A1, ARG1,
GAD1, GLUD1, GOT1, GPT2, GRHPR, IVD, MCCC2, OAT, OTC, OXCT1, PC, PSAT1, SDS
Fatty Acid Alpha Oxidation (Human): HAAO, HACL1, PEX13, PHYH, SLC27A2
Fatty Acid Beta Oxidation (Human): ABCD1, ABCD2, ABCD3, ACAA2, ACACB, ACAD11,
ACADL, ACADM, ACADS, ACADVL, ACAT1, ACAT2, ACOX1, ACOX2, ACOX3, ACOXL,
ACSBG2, ACSL5, ADIPOQ, AKT2, AUH, BDH2, CPT1A, CPT2, CROT, DECR1, ECHDC1,
ECHDC2, ECHS1, ECI1, ECI2, EHHADH, ETFA, ETFB, ETFDH, FABP1, GCDH, HADH,
HADHA, HADHB, HIBCH, HSD17B4, IRS1, IRS2, IVD, LEP, PEX2, PEX5, PEX7, SESN2,
SLC25A17, SLC27A2, TWIST1
Glycolysis (Human): G6PC2, HKDC1, LDHAL6A, LDHAL6B, PFKFB2, PFKFB3, PFKFB4,
PKM, SLC2A1, SLC2A3, SLC2A4, SLC2A5
Oxidative Phosphorylation (Human): ATP5A1, ATP5B, ATP5D, ATP5E, ATP5F1, ATP5G1,
ATP5G2, ATP5G3, ATP5H, ATP5I, ATP5J, ATP5J2, ATP5L, ATP5O, ATP5S, BCS1L, CEP89,
COA1, COA3, COA4, COA5, COA6, COA7, COX10, COX10-AS1, COX11, COX14, COX15,
COX16, COX17, COX18, COX19, COX20, COX4I1, COX4I2, COX5A, COX5B, COX6A1,
COX6A2, COX6B1, COX6B2, COX6C, COX7A1, COX7A2, COX7A2L, COX7B, COX7B2,
COX7C, COX8A, COX8C, CYC1, CYCS, DNAJC15, MT-ATP6, MT-ATP8, MT-CO1, MT-
CO2, MT-CO3, MT-CYB, MT-ND1, MT-ND2, MT-ND3, MT-ND4, MT-ND4L, MT-ND5, MT-
ND6, NDUFA1, NDUFA10, NDUFA11, NDUFA12, NDUFA13, NDUFA2, NDUFA3,
NDUFA4, NDUFA4L2, NDUFA5, NDUFA6, NDUFA7, NDUFA8, NDUFA9, NDUFAB1,
NDUFAF1, NDUFAF2, NDUFAF3, NDUFAF4, NDUFAF5, NDUFAF6, NDUFAF7,
NDUFAF8, NDUFB1, NDUFB10, NDUFB11, NDUFB2, NDUFB2-AS1, NDUFB3, NDUFB4,
NDUFB5, NDUFB6, NDUFB7, NDUFB8, NDUFB9, NDUFC1, NDUFC2, NDUFS1, NDUFS2,
NDUFS3, NDUFS4, NDUFS5, NDUFS6, NDUFS7, NDUFS8, NDUFV1, NDUFV2, NDUFV3,
NUBPL, OXA1L, RFESD, SCO1, SCO2, SLC25A4, SURF1, TACO1, TIMMDC1, TMEM126B,
TRAP1, TTC19, UQCC1, UQCC2, UQCC3, UQCR10, UQCR11, UQCRB, UQCRC1, UQCRC2,
UQCRFS1, UQCRH, UQCRHL, UQCRQ
Pentose Phosphate Signature (Human): G6PD, H6PD, PGD, PRPS1, PRPS1L1, PRPS2, RBKS,
RGN, RPE, RPIA, TALDO1, TKT, TKTL1, TKTL2
TCA Cycle (Human): ACO2, CS, DLAT, DLD, DLST, FH, GLUD1, IDH1, IDH2, IDH3A,
IDH3B, IDH3G, MDH2, MPC1, MPC2, OGDH, OGDHL, PDHA1, PDHA2, PDHB, PDHX,
PDK1, PDK2, PDK3, PDK4, PDP1, PDP2, PDPR, SDHA, SDHAF1, SDHAF2, SDHAF3,
SDHAF4, SDHB, SDHC, SDHD, SUCLA2, SUCLG1, SUCLG2, SUGCT
Anergic or Activated T (Human): CD160, CD244, CTLA4, ICOS, KLRG1, LAG3, PDCD1
B Cell (Human): AICDA, BANK1, BLK, BLNK, CD19, CD22, CD79A, CD79B, CLEC17A,
CLNK, CR2, DAPP1, DTX1, FAM129C, FAM30A, FCRL1, FCRL2, FCRL4, FCRL5, FCRLA,
GAPT, GNG7, GPR183, HLA-DOA, HLA-DOB, IGHD, IGHM, IRF4, LINC00926, LY6D,
MEF2B, MS4A1, MSC, PAX5, PLCL2, POU2AF1, PRKCH, SH2B2, SIGLEC6, ST6GAL1,
TNFRSF13B, TNFRSF13C, VPREB1, VPREB3
Dendritic Cell (Human): CD1B, CD1C, CD1E, CD207, CLEC10A, CLEC9A, GPR31, MRC1,
XCR1
GC B Cell (Human): FCRLB, GCSAM, KLHL6, LRMP, NUGGC, P2RY8, PARPBP, RGS13
Granulocyte (Human): ADGRG5, ARG1, BPI, CAMP, CD177, CLC, CRISP3, CTSS, CXCR2,
DEFA3, FPR2, LTB4R, MMP25, MMP8, MS4A2, OLFM4, OSM, PGLYRP1, PRG2, PRTN3,
S100A12
LDG (Human): AZU1, CEACAM3, CEACAM4, CEACAM5, CEACAM6, CEACAM8, CTSG,
DEFA4, ELANE, MPO, RETN, RNASE3
Monocyte/Myeloid Cell (Human): ACE, ADAM8, ADAMDEC1, ADGRE1, ADGRE2,
ADGRE3, AIF1, AOAH, APOBEC3B, APOBEC3G, APOBR, APOC1, ART4, BMX, C1QA,
C1QB, C1QC, C2, C4A, C4B, C4BPA, C4BPB, C5, C6, C8A, C9, CCL17, CCL18, CCL22,
CCL28, CCL7, CCL8, CD101, CD14, CD163, CD209, CD300C, CD300E, CD300LF, CD33,
CD5L, CD68, CD80, CFB, CFD, CFP, CHI3L1, CHIT1, CLEC12A, CLEC12B, CLEC16A,
CLEC1A, CLEC2B, CLEC4A, CLEC4D, CLEC4E, CLEC4G, CLEC5A, CLEC6A, CLEC7A,
CSF1R, CSF2RA, CSF2RB, CSF3R, CST3, CXCL1, CXCL10, CXCL11, CXCL13, CXCL8,
CXCL9, CYBA, CYBB, F12, FCER1G, FCGR1A, FCGR1B, FCGR2A, FCGR2B, FCGR3A,
FCGR3B, FFAR2, FGR, FLT3, HMMR, HVCN1, IFI30, IFNL1, IFNL2, IFNL3, IGSF6,
IL10RA, IL12A, IL12B, IL1A, IL1B, IL1F10, IL1RAP, IL1RN, IL20, IL23A, IL26, IL27,
IL31RA, IL37, ITGAX, LAMP3, LGALS12, LGALS4, LGALS9, LGALS9B, LGALS9C,
LILRA1, LILRA2, LILRA3, LILRA5, LILRA6, LILRB1, LILRB2, LILRB3, LILRB4, LILRB5,
LMNB1, LY86, LYVE1, LYZ, MARCO, MEFV, MEGF10, MERTK, MFGE8, MNDA, MPEG1,
MS4A4A, MS4A6A, MS4A7, MSR1, NLRP12, NLRP3, NOD2, NTSR1, OCM, OLR1, OSCAR,
OTOF, PDCD1LG2, PECAM1, PILRA, PLA2G2D, PLEK, PRAM1, PYHIN1, RNASE6,
RNASE7, S100A8, S100A9, S1PR5, SCARB1, SCARF2, SECTM1, SEMA4A, SERPINB9,
SERPING1, SIGLEC1, SIGLEC10, SIGLEC14, SIGLEC5, SIGLEC7, SKAP2, SLAMF8,
SLC11A1, SLITRK4, SLPI, SMPDL3B, SPI1, SPIC, STAB2, STAP2, TEK, TGM2, TIMD4,
TLR2, TLR8, TNF, TNFAIP8L2, TNFRSF1B, TNFSF13B, TNFSF14, TNIP3, TREM1,
TREML2, TREML4, TULP1, TYROBP, UBD, UNC93B1, VENTX, VSIG4, VSTM1
NK Cell (Human): KIR2DL4, KLRC3, KLRF1, NCAM1, NCR1, SH2D1B, TNFSF11, TXK
pDC (Human): CLEC4C, IRF7, LILRA4, NRP1, PACSIN1, PLAC8, PTCRA, SERPINF1,
SLC15A4, TCF4
Plasma Cell (Human): DERL3, FANCI, GPRC5D, IGH, IGHA1, IGHA2, IGHD2-15, IGHD2-21,
IGHD3-10, IGHD3-16, IGHD3-3, IGHE, IGHG1, IGHG2, IGHG3, IGHG4, IGHGP, IGHJ1,
IGHJ2, IGHJ3, IGHJ4, IGHJ5, IGHJ6, IGHMBP2, IGHV1-18, IGHV1-2, IGHV1-24, IGHV1-3,
IGHV1-45, IGHV1-46, IGHV1-58, IGHV1-69-2, IGHV2-26, IGHV2-5, IGHV2-70, IGHV3-11,
IGHV3-13, IGHV3-15, IGHV3-16, IGHV3-20, IGHV3-21, IGHV3-23, IGHV3-33, IGHV3-35,
IGHV3-38, IGHV3-43, IGHV3-47, IGHV3-48, IGHV3-49, IGHV3-53, IGHV3-54, IGHV3-7,
IGHV3-72, IGHV3-73, IGHV3-74, IGHV3OR16-8, IGHV4-28, IGHV4-30-2, IGHV4-31,
IGHV4-34, IGHV4-39, IGHV4-59, IGHV4-61, IGHV5-51, IGHV5-78, IGHV6-1, IGHV7-34-1,
IGHV7-81, IGK, IGKC, IGKJ1, IGKJ2, IGKJ3, IGKJ4, IGKJ5, IGKV@, IGKV1-16, IGKV1-17,
IGKV1-27, IGKV1-5, IGKV1-6, IGKV1-9, IGKV1D-16, IGKV1D-17, IGKV1D-27, IGKV1D-
43, IGKV1D-8, IGKV2D-24, IGKV2D-26, IGKV3-20, IGKV3-7, IGKV3D-20, IGKV3D-7,
IGKV4-1, IGKV5-2, IGL, IGLC1, IGLC2, IGLC6, IGLC7, IGLJ3, IGLJ6, IGLL1, IGLL3P,
IGLL5, IGLV@, IGLV10-54, IGLV1-36, IGLV1-40, IGLV1-44, IGLV1-47, IGLV1-50, IGLV2-
11, IGLV2-14, IGLV2-18, IGLV2-23, IGLV2-5, IGLV2-8, IGLV3-1, IGLV3-10, IGLV3-12,
IGLV3-16, IGLV3-19, IGLV3-21, IGLV3-22, IGLV3-25, IGLV3-27, IGLV3-9, IGLV4-3,
IGLV4-60, IGLV4-69, IGLV5-37, IGLV5-45, IGLV5-48, IGLV6-57, IGLV7-43, IGLV7-46,
IGLV8-61, IGLV9-49, IGLVI-56, IGLVI-70, MZB1, PCLAF, TNFRSF17
Platelet (Human): GP1BA, GP9, ITGA2B, PF4, PF4V1, PPBP
T Cell (Human): CD247, CD3D, CD3E, CD3G, CD8A, CD8B, TRAC, TRAT1, TRAV20,
TRBC1, TRDC
Langerhans Cell (Human): CD1B, CD1C, CD1E, CD207
Skin-specific DC (Human): CLEC10A, CLEC9A, GPR31, MRC1, XCR1
Endothelial Cell (Human): DLC1, ECSCR, EMCN, FLT1, KDR, LDB2, LRRC32, MEIS2,
PLAT, PTPRB, SELE, TM4SF1, TM4SF18, VWF
Fibroblast (Human): 43894, ADAM33, ADAMTS6, AGTR1, ALPK2, ANGPTL2, ANKRD45,
ANO2, ANPEP, ARMC9, ASPN, BDKRB2, BDNF, BMPER, C11orf87, C1R, C1S, CATSPER3,
CCDC102B, CCDC80, CCDC81, CEMIP, CHAC1, CHRM2, CLMP, CNN1, COL14A1,
COL3A1, COL5A1, COLEC10, CPXM2, CPZ, CRABP2, CXCL12, DDR2, DKK1, DMRTA1,
EGFL6, ELOVL2, EMILIN1, FAM180A, FBLN7, FBN1, FGF5, FMN2, FOXF2, FST, FSTL1,
GFRA1, GLIS1, GLT8D2, GPR176, GREM1, GREM2, GRIK2, GUCY1A2, HSD17B2, HSPA2,
HSPB3, IL19, IQCD, KCNMB2, KIRREL3, KRT34, KRTAP1-5, KRTAP3-1, L1CAM, LAYN,
LMOD1, LOXL4, LUM, LY6K, MFAP4, MFAP5, MGARP, MGP, MKX, MMP2, MXRA5,
MXRA8, MYPN, NEXN, NFASC, NID2, NTF3, OLFML3, P3H3, P4HA3, PAMR1, PAX3,
PCDHGA2, PCDHGA3, PCDHGA7, PDE8B, PDGFRA, PDGFRL, PDZRN3, PLA2R1,
PLEKHA4, PLPP4, PLPPR4, PRKG1, PSG5, PTPRQ, PTRF, SEMA3A, SEMA5A, SEMA6D,
SHOX, SLC16A2, SMIM2, SPARCL1, SPHKAP, SSTR1, STC1, STXBP6, SUSD5, SVEP1,
TBX15, THBS2, TIMP2, TIMP3, TMEM119, TMEM130, TMEM47, TRHDE, TRPC4, UACA,
UBL4B, VAT1L, VEGFC, WNT5A, WNT5B, ZFPM2
Keratinocyte (Human): ABLIM, AKR1C1, ALDH8, ALOX12B, ANXA8, AQP3, ATDC,
BPAG1, CA12, CCND2, CD24, CDH3, CDKN1A, CDSN, COL17A1, CST6, CSTA, DD96,
DSC1, DSG1, DSP, EGFR, EVPL, FLG, G0S2, GJA1, GLUL, GNA15, HBP17, IFI27, IFITM1,
IGFBP7, ITGA3, ITGB4, IVL, JUNB, JUP, KLK11, KLK7, KLK8, KRT1, KRT14, KRT15,
KRT16, KRT2A, KRT5, KRT6A, LAMA3, LGALS7, LOR, NOTCH3, PPL, PRDX2, PRSS11,
PRSS2, PRSS4, S100A2, SERPINB2, SERPINB3, SERPINB4, SERPINB5, SERPINB7,
SERPINE1, SPINK5, SPRR1B, TACSTD2, TFAP2A, TGM1, TP63, TUBA1, XP5
Kidney Cell (Human): A2LD1, ACE2, ACOT4, ACP6, ACSF2, ADCY5, ADGRF1, ADHFE1,
AGBL3, AGMAT, AGR2, AJAP1, AKR7A3, ALDH8A1, ARL4C, ARSE, ARSF, BICC1,
BICDL1, C16orf11, C21orf62, C6orf47, CALCRL, CCDC106, CCDC160, CD24, CDHR5,
CDKL2, CHDH, CHST9, CLDN10, CLDN16, CLDN2, CLRN3, COL4A3, COL4A4, COL4A5,
COMTD1, COQ8A, CPVL, CTXN3, CXorf57, CYP27B1, CYP4A22, CYS1, DAO, DCDC2,
DCXR, DHDH, DMGDH, EFHD1, EME2, EN2, ERVMER34-1, ESM1, ETNK2, FAM110C,
FAM134B, FAM151A, FAM184A, FANK1, FNDC8, FOXII, FRMD7, FTCD, G6PC, GGH,
GLOD5, GNE, HABP2, HAO2, HAVCR1, HEY1, HIST1H4D, HMX1, HNF1A, HNF1B,
HOGA1, HOOK1, HOXD3, HPD, HS3ST1, HS6ST2, HSPA12A, HYKK, IL17RB, INSRR,
IRX5, ITGB8, KCNH6, KCNJ11, KCTD14, KIF12, LAMB2, LGSN, LHX1, LIPG, LRP2,
LRRC19, MDC1, MZF1, NME5, NOS1, NR1H4, OR2T10, OSBPL10, OVGP1, P3H2, PADI1,
PANK1, PAX8, PCK1, PCSK5, PDZD3, PDZK1IP1, PKHD1, PKLR, PLEKHA1, PLLP,
PLPPR1, POU3F3, PPT2, PRKAA2, PRODH, PRODH2, PROM1, PROZ, PRRG1, PTCHD1,
RALYL, RANBP3L, RARB, RARRES3, RASIP1, RBM20, REN, RHBG, RIDA, RNF182,
RNF212B, RPRM, RSPH1, RYR3, SALL1, SCEL, SCNN1G, SFXN2, SGK2, SGPP2, SHISA3,
SIM2, SLC10A2, SLC13A3, SLC16A4, SLC16A5, SLC17A1, SLC17A3, SLC22A11,
SLC22A24, SLC22A6, SLC22A7, SLC28A1, SLC30A2, SLC34A3, SLC3A1, SLC47A2,
SLC4A4, SLC6A19, SLC7A13, SLC7A7, SLC7A9, SLCO4A1, SMTNL2, SOD3, SOSTDC1,
SPON1, SYT9, SYTL2, TAGLN3, TLN2, TMEM207, TMPRSS4, TPD52L1, TRABD2B,
TRPV5, TSPAN1, TSPAN12, UGT1A9, UGT2A3, UGT3A1, UGT8, UPK1B, UPP2, VEPH1,
WIF1, WNK4, XPNPEP2, ZBED2, ZNF324B, ZNF331, ZNF837, ZPLD1, ZSCAN18
Kidney Distal Tubule (Human): ATP6V0A4, ATP6V0D2, C14orf105, CALB1, CDH1, CDH16,
CLCNKA, CLDN8, EMX1, EPCAM, KCNJ1, PTH1R, PVALB, SCIN, SCNN1A, SIM1,
SLC12A1, SLC6A13, SULT1C2, TFCP2L1, TMEM213, TMEM72, UMOD, WFDC2
Kidney Loop of Henle Cell (Human): CD9, CLCNKB, KCNJ16, KNG1, S100A6, SPP1
Kidney Proximal Convoluted Tubule (Human): ACSM2A, ACSM2B, ACY1, ANK2, AQP1,
ASS1, BBOX1, BHMT, BHMT2, C10orf126, CMBL, CRYAA, CRYAB, CUBN, CXCL14,
DDAH1, FMO1, GALNT14, GLYAT, GPX3, MIOX, NAT8, NOX4, PAH, PAX2, PDZK1,
RAB11FIP3, RNF152, SLC12A3, SLC13A1, SLC16A12, SLC22A12, SLC22A13, SLC22A2,
SLC22A8, SLC34A1, SLC36A2, SLC5A2, SLC6A18, TMEM174, TMEM52B, UGT2B7
Kidney Tubule Collecting Duct Cell (Human): AQP2, CLDN4, COL4A1, CPE, CYR61, FXYD4,
GLIS3, MACC1, MITF, PIGR, SLC4A9, WWC1
Melanocyte (Human): ASIP, CITED1, DCT, GPNMB, GPR143, MITF, MLANA, MLPH, OCA2,
PMEL, SLC24A5, SLC45A2, TYR, TYRP1
Mesangial Cell (Human): ACTA2, ACTN1, ADAMTSL4, AKAP12, AMOTL2, ANGPT2,
ARID5B, CALD1, CSTB, CTGF, DYRK1A, ECM1, FGF1, FILIP1L, FLNA, FN1, GATA3,
HOPX, ITGA8, LATS2, LRP5, MAP3K20, MEF2C, MIB1, NCOA2, NOCT, NR4A2, OGT,
PAWR, PDE3A, PDGFB, PDGFRB, PER3, PLAU, PML, PTN, RASD1, ROCK1, SERPINE2,
SFRP2, SH3BGRL, SMARCA5, SNCG, TBX18, THY1, TNS1, TPM2, TRIP4, YAP1
Podocyte (Human): NPHS1, NPHS2, PODXL, SYNPO, WT1
Inflammatory Cell (combined Aner/Act T, B Cell, DCI, Monocyte/MC, NK Cell, pDC, Plasma
Cell, Skin-specific DC, T Cell) (Human): CD160, CD244, CTLA4, ICOS, KLRG1, LAG3,
PDCD1, AICDA, BANK1, BLK, BLNK, CD19, CD22, CD79A, CD79B, CLEC17A, CLNK,
CR2, DAPP1, DTX1, FAM129C, FAM30A, FCRL1, FCRL2, FCRL4, FCRL5, FCRLA, GAPT,
GNG7, GPR183, HLA-DOA, HLA-DOB, IGHD, IGHM, IRF4, LINC00926, LY6D, MEF2B,
MS4A1, MSC, PAX5, PLCL2, POU2AF1, PRKCH, SH2B2, SIGLEC6, ST6GAL1,
TNFRSF13B, TNFRSF13C, VPREB1, VPREB3, CD1B, CD1C, CD1E, CD207, CLEC10A,
CLEC9A, GPR31, MRC1, XCR1, ACE, ADAM8, ADAMDEC1, ADGRE1, ADGRE2,
ADGRE3, AIF1, AOAH, APOBEC3B, APOBEC3G, APOBR, APOC1, ART4, BMX, C1QA,
C1QB, C1QC, C2, C4A, C4B, C4BPA, C4BPB, C5, C6, C8A, C9, CCL17, CCL18, CCL22,
CCL28, CCL7, CCL8, CD101, CD14, CD163, CD209, CD300C, CD300E, CD300LF, CD33,
CD5L, CD68, CD80, CFB, CFD, CFP, CHI3L1, CHIT1, CLEC12A, CLEC12B, CLEC16A,
CLEC1A, CLEC2B, CLEC4A, CLEC4D, CLEC4E, CLEC4G, CLEC5A, CLEC6A, CLEC7A,
CSF1R, CSF2RA, CSF2RB, CSF3R, CST3, CXCL1, CXCL10, CXCL11, CXCL13, CXCL8,
CXCL9, CYBA, CYBB, F12, FCER1G, FCGR1A, FCGR1B, FCGR2A, FCGR2B, FCGR3A,
FCGR3B, FFAR2, FGR, FLT3, HMMR, HVCN1, IFI30, IFNL1, IFNL2, IFNL3, IGSF6,
IL10RA, IL12A, IL12B, IL1A, IL1B, IL1F10, IL1RAP, IL1RN, IL20, IL23A, IL26, IL27,
IL31RA, IL37, ITGAX, LAMP3, LGALS12, LGALS4, LGALS9, LGALS9B, LGALS9C,
LILRA1, LILRA2, LILRA3, LILRA5, LILRA6, LILRB1, LILRB2, LILRB3, LILRB4, LILRB5,
LMNB1, LY86, LYVE1, LYZ, MARCO, MEFV, MEGF10, MERTK, MFGE8, MNDA, MPEG1,
MS4A4A, MS4A6A, MS4A7, MSR1, NLRP12, NLRP3, NOD2, NTSR1, OCM, OLR1, OSCAR,
OTOF, PDCD1LG2, PECAM1, PILRA, PLA2G2D, PLEK, PRAM1, PYHIN1, RNASE6,
RNASE7, S100A8, S100A9, S1PR5, SCARB1, SCARF2, SECTM1, SEMA4A, SERPINB9,
SERPING1, SIGLEC1, SIGLEC10, SIGLEC14, SIGLEC5, SIGLEC7, SKAP2, SLAMF8,
SLC11A1, SLITRK4, SLPI, SMPDL3B, SPI1, SPIC, STAB2, STAP2, TEK, TGM2, TIMD4,
TLR2, TLR8, TNF, TNFAIP8L2, TNFRSF1B, TNFSF13B, TNFSF14, TNIP3, TREM1,
TREML2, TREML4, TULP1, TWIST1, TYROBP, UBD, UNC93B1, VENTX, VSIG4, VSTM1,
KIR2DL4, KLRC3, KLRF1, NCAM1, NCR1, SH2D1B, TNFSF11, TXK, CLEC4C, IRF7,
LILRA4, NRP1, PACSIN1, PLAC8, PTCRA, SERPINF1, SLC15A4, TCF4, DERL3, FANCI,
GPRC5D, IGH, IGHA1, IGHA2, IGHD2-15, IGHD2-21, IGHD3-10, IGHD3-16, IGHD3-3,
IGHE, IGHG1, IGHG2, IGHG3, IGHG4, IGHGP, IGHJ1, IGHJ2, IGHJ3, IGHJ4, IGHJ5, IGHJ6,
IGHMBP2, IGHV1-18, IGHV1-2, IGHV1-24, IGHV1-3, IGHV1-45, IGHV1-46, IGHV1-58,
IGHV1-69-2, IGHV2-26, IGHV2-5, IGHV2-70, IGHV3-11, IGHV3-13, IGHV3-15, IGHV3-16,
IGHV3-20, IGHV3-21, IGHV3-23, IGHV3-33, IGHV3-35, IGHV3-38, IGHV3-43, IGHV3-47,
IGHV3-48, IGHV3-49, IGHV3-53, IGHV3-54, IGHV3-7, IGHV3-72, IGHV3-73, IGHV3-74,
IGHV3OR16-8, IGHV4-28, IGHV4-30-2, IGHV4-31, IGHV4-34, IGHV4-39, IGHV4-59,
IGHV4-61, IGHV5-51, IGHV5-78, IGHV6-1, IGHV7-34-1, IGHV7-81, IGK, IGKC, IGKJ1,
IGKJ2, IGKJ3, IGKJ4, IGKJ5, IGKV@, IGKV1-16, IGKV1-17, IGKV1-27, IGKV1-5, IGKV1-6,
IGKV1-9, IGKV1D-16, IGKV1D-17, IGKV1D-27, IGKV1D-43, IGKV1D-8, IGKV2D-24,
IGKV2D-26, IGKV3-20, IGKV3-7, IGKV3D-20, IGKV3D-7, IGKV4-1, IGKV5-2, IGL, IGLC1,
IGLC2, IGLC6, IGLC7, IGLJ3, IGLJ6, IGLL1, IGLL3P, IGLL5, IGLV@, IGLV10-54, IGLV1-
36, IGLV1-40, IGLV1-44, IGLV1-47, IGLV1-50, IGLV2-11, IGLV2-14, IGLV2-18, IGLV2-23,
IGLV2-5, IGLV2-8, IGLV3-1, IGLV3-10, IGLV3-12, IGLV3-16, IGLV3-19, IGLV3-21, IGLV3-
22, IGLV3-25, IGLV3-27, IGLV3-9, IGLV4-3, IGLV4-60, IGLV4-69, IGLV5-37, IGLV5-45,
IGLV5-48, IGLV6-57, IGLV7-43, IGLV7-46, IGLV8-61, IGLV9-49, IGLVI-56, IGLVI-70,
MZB1, PCLAF, TNFRSF17, CLEC10A, CLEC9A, GPR31, MRC1, XCR1, CD247, CD3D,
CD3E, CD3G, CD8A, CD8B, TRAC, TRAT1, TRAV20, TRBC1, TRDC
Apoptotic Mitochondrial Changes (Human): BAK1, BAX, BBC3, BCL2L1, BID, CDKN2A,
GPX1, IFI6, PMAIP1, SFN
GO Mitochondrial Fission (Human): BNIP3, DCN, DDHD1, DDHD2, DHODH, DNM1, DNM2,
DNM3, FIS1, GDAP1, GGNBP1, INF2, LRRK2, MAPT, MARCHF5, MFF, MIEF1, MIEF2,
MTFP1, MTFR1, MTFR1L, MTFR2, MUL1, MX1, MX2, MYO19, OPA1, PINK1, PPARGC1A,
PRKN, SLC25A46, STAT2, TMEM135, VPS35
GO Peroxiome Fission (Human): ACOT8, CRYZL2P-SEC16B, DNM1L, PEX11A, PEX11B,
PEX11G, SEC16B
Mitochondrial Large Ribosome (Human): MALSU1, MRM1, MRM3, MRPL1, MRPL10,
MRPL11, MRPL12, MRPL13, MRPL14, MRPL15, MRPL16, MRPL17, MRPL18, MRPL19,
MRPL2, MRPL20, MRPL21, MRPL22, MRPL23, MRPL24, MRPL27, MRPL28, MRPL3,
MRPL30, MRPL32, MRPL33, MRPL34, MRPL35, MRPL36, MRPL37, MRPL38, MRPL39,
MRPL4, MRPL40, MRPL41, MRPL42, MRPL43, MRPL44, MRPL45, MRPL46, MRPL47,
MRPL48, MRPL49, MRPL50, MRPL51, MRPL52, MRPL53, MRPL54, MRPL55, MRPL57,
MRPL58, MRPL9, NSUN4
Mitochondrial Small Ribosome (Human): METTL17, MRPS10, MRPS11, MRPS12, MRPS14,
MRPS15, MRPS16, MRPS17, MRPS18A, MRPS18B, MRPS18C, MRPS2, MRPS21, MRPS22,
MRPS23, MRPS24, MRPS25, MRPS26, MRPS27, MRPS28, MRPS30, MRPS31, MRPS33,
MRPS34, MRPS35, MRPS36, MRPS5, MRPS6, MRPS7, MRPS9
Mitochondrial Transcription (Human): CLUH, FASTKD5, LRPPRC, MTERF1, MTERF2,
MTERF3, MTERF4, MTPAP, MTRES1, NT5M, POLG, POLG2, POLRMT, PRIMPOL, SSBP1,
SUPV3L1, TEFM, TFAM, TFB1M, TFB2M, TOP1MT
Mitochondrial Translation (Human): AARS2, C12orf65, DARS2, DDX28, EARS2, FARS2,
GATB, GATC, GFM1, GFM2, GRSF1, GTPBP3, GUF1, HARS2, HEMK1, IARS2, LARS2,
MARS2, METAP1D, MPV17L2, MRM2, MRRF, MTFMT, MTG1, MTG2, MTIF2, MTIF3,
MTO1, MTRF1, MTRF1L, NARS2, PARS2, PTCD1, PTCD2, PTCD3, PTRH1, PTRH2,
QRSL1, RARS2, RMND1, SARS2, TARS2, TRIT1, TRMT10C, TRMT61B, TRMU, TRNT1,
TSFM, TUFM, VARS2, WARS2, YARS2
Peroxiome Biogenesis (Human): LONP2, PEX1, PEX10, PEX12, PEX14, PEX16, PEX19,
PEX26, PEX3, PEX5L, PEX6, PIPOX, PXMP2, PXMP4, VPS4B
Mouse Amino Acid Metabolism: Aass, Agxt, Agxt2, Aldh4a1, Aldh7a1, Arg1, Gad1, Glud1,
Got1, Gpt2, Grhpr, Ivd, Mccc2, Oat, Otc, Oxct1, Pcx, Psat1, Sds
Mouse Fatty Acid Alpha Oxidation: Haao, Hacl1, Pex13, Phyh, Slc27a2
Mouse Fatty Acid Beta Oxidation: Abcd1, Abcd2, Abcd3, Acaa2, Acacb, Acad11, Acadl, Acadm,
Acads, Acadv1, Acat1, Acat3, Acox1, Acox2, Acox3, Acoxl, Acsbg2, Acsl5, Adipoq, Akt2, Auh,
Bdh2, Cpt1a, Cpt2, Crot, Decr1, Echdc1, Echdc2, Echs1, Eci1, Eci2, Ehhadh, Etfa, Etfb, Etfdh,
Fabp1, Gcdh, Hadh, Hadha, Hadhb, Hibch, Hsd17b4, Irs1, Irs2, Ivd, Lep, Pex2, Pex5, Pex7,
Sesn2, Slc25a17, Slc27a2, Twist1
Mouse Glycolysis: G6pc2, Hkdc1, Ldhal6b, Pfkfb2, Pfkfb3, Pfkfb4, Pkm, Slc2a1, Slc2a3, Slc2a4,
Slc2a5
Mouse Oxidative Phosphorylation: Atp5a1, Atp5b, Atp5d, Atp5f1, Atp5g1, Atp5g2, Atp5g3,
Atp5h, Atp5j, Atp5j2, Atp5l, Atp5o, Atp5s, Bcs1l, Cep89, Coa3, Coa4, Coa5, Coa7, Cox10,
Cox11, Cox14, Cox15, Cox16, Cox17, Cox18, Cox19, Cox20, Cox4i1, Cox4i2, Cox5a, Cox6a1,
Cox6a2, Cox6b1, Cox6b2, Cox7a1, Cox7a2, Cox7a2l, Cox7b, Cox8a, Cyc1, Cycs, Dnajc15,
Gm9763, LOC102631912, LOC102640946, Ndufa1, Ndufa10, Ndufa11, Ndufa12, Ndufa13,
Ndufa2, Ndufa3, Ndufa4, Ndufa4l2, Ndufa5, Ndufa6, Ndufa7, Ndufa8, Ndufa9, Ndufab1,
Ndufaf1, Ndufaf2, Ndufaf3, Ndufaf4, Ndufaf5, Ndufaf6, Ndufaf7, Ndufb10, Ndufb11, Ndufb2,
Ndufb3, Ndufb4, Ndufb5, Ndufb6, Ndufb7, Ndufb8, Ndufb9, Ndufc1, Ndufc2, Ndufs1, Ndufs2,
Ndufs3, Ndufs4, Ndufs6, Ndufs7, Ndufs8, Ndufv1, Ndufv2, Ndufv3, Nubpl, Oxa1l, Rfesd, Sco1,
Sco2, Slc25a4, Surf1, Taco1, Timmdc1, Tmem126b, Trap1, Ttc19, Uqcc1, Uqcc2, Uqcr10,
Uqcr11, Uqcrb, Uqcrc1, Uqcrc2, Uqerfs1, Uqcrh, Uqcrq
Mouse PPP: G6pdx, H6pd, Pgd, Prps1, Prps1l1, Prps2, Rbks, Rgn, Rpe, Rpia, Taldo1, Tkt, Tktl1,
Tktl2
Mouse TCA Cycle: Aco2, Cs, Dlat, Dld, Dlst, Fh1, Glud1, Idh1, Idh2, Idh3a, Idh3b, Idh3g, Mdh2,
Mpc1, Mpc2, Ogdh, Ogdhl, Pdha1, Pdhb, Pdhx, Pdk1, Pdk2, Pdk3, Pdk4, Pdp1, Pdp2, Pdpr, Sdha,
Sdhaf1, Sdhaf2, Sdhb, Sdhc, Sdhd, Sucla2, Suclg1, Suclg2, Sugct
Mouse Anergic/Activated T Cell with T: Ctla4, Lag3, Pdcd1, Cd3e, Cd3g, Cd6, Cd8a, Cd8b1
Mouse Anergic/Activated T Cell: Ctla4, Lag3, Pdcd1
Mouse B Cell: Aicda, Bank1, Blk, Blnk, Cd19, Cd22, Cd72, Cd79a, Cd79b, Cr2, Ctnnbl1, Dapp1,
Fam129c, Fcer2a, Fcrl1, Fcrl5, Gapt, H2-Oa, H2-Ob, Ighd, Ighm, Igll1, Irf4, Ly6d, Ms4a1, Pax5,
Plcl2, Pou2af1, Pou2f2, Prkch, Sh2b2, Siglecg, St6gal1, Tnfrsf13b, Tnfrsf13c, Vpreb1, Vpreb3
Mouse Dendritic Cell: Batf3, Cd209a, Cd209e, Clec1a, Clec9a, Itgax, Ly75, Pdcd1lg2, Tnfrsf11a,
Vcam1, Xcr1
Mouse GC B Cell: Gcsam, Lrmp, Mybl1, Klhl6, Rgs13, Fcrlb, Nuggc
Mouse Granulocyte: Akna, Camp, Cd177, Cxcr3, Fpr2, Fut7, Ltb4r1, Mefv, Mmp25, Mmp8,
Ms4a2, Olfm4, Pglyrp1, Pram1, Prg2, Prtn3
Mouse LDG: Ctsg, Mpo, Osm, Elane
Mouse Monocyte/Myeloid Cell: Ace, Adam8, Adgre1, Aif1, Aoah, Apobr, Apoc1, Batf2, Bmx,
Bpi, C1qa, C1qb, C1qc, C2, C4b, C6, Ccl17, Ccl28, Ccl3, Ccl7, Ccl8, Cd14, Cd163, Cd300e,
Cd300lf, Cd33, Cd5l, Cd68, Cd80, Cfb, Cfd, Cfp, Chil1, Clec12a, Clec4a2, Clec4b1, Clec4d,
Clec4e, Clec4g, Clec4n, Clec5a, Clec7a, Csf1r, Csf2ra, Csf2rb, Csf3r, Cst3, Cxcl1, Cxcl10,
Cxcl11, Cxcl13, Cxcl9, Cyba, Cybb, F12, Fcer1a, Fcer1g, Fcgr1, Fcgr3, Fcgr4, Ffar2, Fgr, Flt3,
Folr2, Gm15448, Hc, Hvcn1, Ifi209, Ifi211, Ifnl2, Ifnl3, Igsf6, Il10ra, Il12a, Il12b, Il18, Il1a, Il1b,
Il1rap, Il1rn, Il23a, Il27, Il31ra, Lgals12, Lgals9, Lilrb4a, Ly86, Lyve1, Lyz2, Marco, Megf10,
Mertk, Mfge8, Mgl2, Mpeg1, Mrc1, Ms4a4a, Ms4a6d, Msr1, Nlrp12, Nlrp3, Nod2, Npy1r, Olr1,
Oscar, P2rx5, Pecam1, Pilra, Pla2g2d, Pla2g5, Plac8, Rnase6, S100a8, S100a9, Scarb1, Scarf2,
Sectm1a, Sema4a, Serping1, Siglec1, Slamf8, Slpi, Tlr1, Trem1, Treml4, Tyrobp, Vsig4
Mouse NK Cell: Itga2, Klra10, Klra3, Klra8, Klra9, Klrb1a, Ncr1, Serpinb9b, Sh2d1b1, Klrb1b,
S1pr5, Adamts14, Klre1, Styk1
Mouse pDC: Bst2, Cd300c, Siglech
Mouse Plasma Cell: Derl3, Igha, Ighe, Ighg1, Ighg2a, Ighg2b, Ighg2c, Ighg3, Ighj2, Ighj3, Ighj4,
Ighv10-1, Ighv10-3, Ighv11-1, Ighv11-2, Ighv1-12, Ighv1-14, Ighv1-15, Ighv1-19, Ighv1-20,
Ighv1-22, Ighv12-3, Ighv1-26, Ighv1-31, Ighv13-2, Ighv1-34, Ighv1-36, Ighv1-37, Ighv1-39,
Ighv1-4, Ighv14-1, Ighv14-3, Ighv1-43, Ighv14-4, Ighv1-47, Ighv1-49, Ighv1-5, Ighv1-50,
Ighv15-2, Ighv1-52, Ighv1-59, Ighv1-61, Ighv1-62-1, Ighv1-62-2, Ighv1-63, Ighv1-66, Ighv1-7,
Ighv1-71, Ighv1-75, Ighv1-76, Ighv1-77, Ighv1-78, Ighv1-80, Ighv1-81, Ighv1-82, Ighv1-85,
Ighv1-9, Ighv2-2, Ighv2-3, Ighv2-4, Ighv2-5, Ighv2-6, Ighv2-6-8, Ighv2-9, Ighv2-9-1, Ighv3-1,
Ighv3-4, Ighv3-5, Ighv3-6, Ighv3-8, Ighv4-1, Ighv5-12-4, Ighv5-9-1, Ighv6-3, Ighv6-6, Ighv6-7,
Ighv7-1, Ighv7-3, Ighv7-4, Ighv8-11, Ighv8-12, Ighv8-6, Ighv9-1, Ighv9-2, Ighv9-3, Ighv9-4, Igk,
Igkc, Igkj1, Igkj2, Igkj4, Igkj5, Igkv10-94, Igkv10-95, Igkv1-110, Igkv11-125, Igkv1-117, Igkv1-
122, Igkv1-132, Igkv1-133, Igkv1-135, Igkv12-38, Igkv12-41, Igkv12-44, Igkv12-46, Igkv12-89,
Igkv12-98, Igkv13-84, Igkv13-85, Igkv14-100, Igkv14-111, Igkv14-130, Igkv16-104, Igkv17-
121, Igkv17-127, Igkv18-36, Igkv1-88, Igkv1-99, Igkv19-93, Igkv20-101-2, Igkv2-109, Igkv2-
112, Igkv3-1, Igkv3-10, Igkv3-12, Igkv3-2, Igkv3-3, Igkv3-4, Igkv3-5, Igkv3-7, Igkv3-9, Igkv4-
50, Igkv4-51, Igkv4-53, Igkv4-55, Igkv4-57, Igkv4-57-1, Igkv4-58, Igkv4-59, Igkv4-61, Igkv4-
63, Igkv4-68, Igkv4-69, Igkv4-70, Igkv4-71, Igkv4-72, Igkv4-73, Igkv4-74, Igkv4-78, Igkv4-79,
Igkv4-80, Igkv4-81, Igkv4-86, Igkv4-90, Igkv4-91, Igkv4-92, Igkv5-37, Igkv5-39, Igkv5-43,
Igkv5-45, Igkv5-48, Igkv6-14, Igkv6-15, Igkv6-17, Igkv6-20, Igkv6-23, Igkv6-25, Igkv6-29,
Igkv6-32, Igkv7-33, Igkv8-16, Igkv8-19, Igkv8-21, Igkv8-24, Igkv8-27, Igkv8-28, Igkv8-30,
Igkv8-34, Igkv9-120, Igkv9-123, Igkv9-124, Iglc1, Iglc2, Iglc3, Iglj1, Iglj2, Iglj3, Iglv1, Iglv2,
Iglv3, Mzb1, Sdc1, Tnfrsf17
Mouse Platelet: Gp1ba, Gp5, Cxcl5, Gp9, Plek, Pf4, Ppbp, Esam, Mmrn1, Treml1, Pear1,
Slc35d3, Myl9, Tspan9, Ly6g6d, Gp6
Mouse T Cell: Cd3d, Cd3e, Cd3g, Cd6, Cd8a, Cd8b1
Mouse Collecting Duct: Aqp2, Cldn4, Col4a1, Cpe, Cyr61, Fxyd4, Glis3, Macc1, Mitf, Pigr,
Slc4a9, Wwc1
Mouse Distal Tubule: 1700011H14Rik, Atp6v0a4, Atp6v0d2, Calb1, Cdh1, Cdh16, Clcnkb,
Cldn8, Emx1, Epcam, Kcnj1, Pth1r, Pvalb, Scin, Scnn1a, Sim1, Slc12a1, Slc6a13, Sult1c2,
Tfcp2l1, Tmem213, Tmem72, Umod, Wfdc2
Mouse Endothelial Cell: Dlc1, Ecscr, Emcn, Flt1, Kdr, Ldb2, Lrrc32, Meis2, Plat, Ptprb, Sele,
Tm4sf1, Vwf
Mouse Fibroblast: 43894, 9930013L23Rik, Adam33, Adamts6, Agtr1a, AI593442, Alpk2,
Angptl2, Ankrd45, Ano2, Anpep, Armc9, Aspn, Bdkrb2, Bdnf, Bmper, C1ra, C1s, Catsper3,
Ccdc80, Ccdc81, Chac1, Chrm2, Clmp, Cnn1, Col14a1, Col3a1, Col5a1, Colec10, Cpxm2, Cpz,
Crabp2, Cxcl12, D3Bwg0562e, Ddr2, Dkk1, Dmrta1, Egfl6, Elovl2, Emilin1, Fam180a, Fbln7,
Fbn1, Fgf5, Fmn2, Foxf2, Fst, Fstl1, Gfra1, Glis1, Glt8d2, Gpr176, Grem1, Grem2, Grik2,
Gucy1a2, Hsd17b2, Hspa2, Hspb3, Il19, Iqcd, Kcnmb2, Kirrel3, Krt34, Krtap1-4, Krtap3-1,
L1cam, Layn, Leprel2, Lmod1, Loxl4, Lum, Ly6k, Mfap4, Mfap5, Mgarp, Mgp, Mkx, Mmp2,
Mxra8, Mypn, Nexn, Nfasc, Nid2, Ntf3, Olfml3, P4ha3, Pamr1, Pax3, Pcdhga2, Pcdhga3,
Pcdhga7, Pde8b, Pdgfra, Pdgfrl, Pdzrn3, Pla2r1, Plekha4, Ppapdc1a, Prkg1, Ptprq, Ptrf, Sema3a,
Sema5a, Sema6d, Slc16a2, Sparcl1, Sphkap, Sstr1, Stc1, Stxbp6, Susd5, Svep1, Tbx15, Thbs2,
Timp2, Timp3, Tmem119, Tmem130, Tmem47, Trhde, Trpc4, Uaca, Ubl4b, Vat1l, Vegfc,
Wnt5a, Wnt5b, Zfpm2
Mouse Kidney Cell: 4932438H23Rik, Ace2, Acot4, Acp6, Acsf2, Adcy5, Adgrf1, Adhfe1, Agbl3,
Agmat, Agr2, Ajap1, Akr7a5, Aldh8a1, Arl4c, Bicc1, Calcrl, Ccdc106, Ccdc160, Cdhr5, Cdkl2,
Chdh, Chst9, Cldn10, Cldn16, Cldn2, Clrn3, Col4a3, Col4a4, Col4a5, Comtd1, Cpvl, Ctxn3,
Cyp27b1, Cyp4a12a, Cys1, D17H6S53E, D330045A20Rik, Dao, Dcdc2a, Dcxr, Dhdh, Dmgdh,
Efhd1, Eme2, En2, Esm1, Etnk2, Fam110c, Fam134b, Fam151a, Fam184a, Fank1, Fndc8, Foxi1,
Frmd7, Ftcd, G6pc, Ggact, Ggh, Glod5, Gne, Habp2, Hao2, Hey1, Hist1h4m, Hmx1, Hnf1a,
Hnf1b, Hoga1, Hook1, Hoxd3, Hpd, Hs3st1, Hs6st2, Hspa12a, Hykk, Il17rb, Insrr, Itgb8, Kcnh6,
Kcnj11, Kctd14, Kif12, Lamb2, Lgsn, Lhx1, Lipg, Lrp2, Lrrc19, Mdc1, Mzf1, Nme5, Nos1,
Nr1h4, Osbpl10, Ovgp1, Padi1, Pank1, Pax8, Pck1, Pcsk5, Pdzd3, Pdzk1ip1, Pipox, Pkhd1, Pklr,
Plekha1, Pllp, Pou3f3, Ppt2, Prkaa2, Prodh, Prodh2, Prom1, Proz, Prrg1, Ptchd1, Ralyl, Ranbp3l,
Rarb, Rasip1, Rbm20, Ren1, Rhbg, Rnf182, Rprm, Rsph1, Ryr3, Sall1, Scel, Scnn1g, Sfxn2,
Sgk2, Sgpp2, Shisa3, Sim2, Slc10a2, Slc13a3, Slc16a4, Slc16a5, Slc17a1, Slc17a3, Slc22a19,
Slc22a6, Slc22a7, Slc28a1, Slc30a2, Slc34a3, Slc3a1, Slc4a4, Slc6a19, Slc7a13, Slc7a7, Slc7a9,
Slco4a1, Smtnl2, Sod3, Sostdc1, Spon1, Syt9, Sytl2, Tagln3, Tln2, Tmem207, Tmprss4, Tpd52l1,
Trabd2b, Trpv5, Tspan1, Tspan12, Ugt1a9, Ugt2a3, Ugt3a2, Ugt8a, Upk1b, Upp2, Veph1, Wif1,
Wnk4, Xpnpep2, Zpld1, Zscan18
Mouse LOH: Cd9, Clcnka, Kcnj16, Kng1, S100a6, Spp1
Mouse Mesangial Cell: Acta2, Actn1, Adamtsl4, Akap12, Amotl2, Angpt2, Arid5b, Cald1, Cstb,
Ctgf, Dyrk1a, Ecm1, Fgf1, Filip1l, Flna, Fn1, Gata3, Hopx, Itga8, Lats2, Lrp5, Map3k20, Mef2c,
Mib1, Ncoa2, Noct, Nr4a2, Ogt, Pawr, Pde3a, Pdgfb, Pdgfrb, Per3, Plau, Pml, Ptn, Rasd1, Rock1,
Serpine2, Sfrp2, Sh3bgrl, Smarca5, Sncg, Tbx18, Thy1, Tns1, Tpm2, Trip4, Yap1
Mouse Podocyte: Nphs1, Nphs2, Podxl, Synpo, Wt1
Mouse Proximal Tubule: Acsm2, Acy1, Ank2, Aqp1, Ass1, Bbox1, Bhmt, Bhmt2, Cmbl, Cryaa,
Cryab, Cubn, Cxcl14, Ddah1, Fmo1, Galnt14, Glyat, Gpx3, Miox, Nox4, Pah, Pax2, Pdzk1,
Rab11fip3, Rnf152, Slc12a3, Slc13a1, Slc16a12, Slc22a12, Slc22a13, Slc22a2, Slc22a8, Slc34a1,
Slc36a2, Slc5a2, Slc6a18, t8, Tmem174, Tmem52b, Ugt2b35
Mouse Type I IFN Core Signature: Acsl1, Adar, Agt, Aim2, Akap2, Apobec3, Atf3, Atf5, Bag1,
Bard1, Bcl7b, Blvra, Brca1, Brca2, Bst2, Cad, Camk2a, Casp1, Cbr1, Cbwd1, Ccl2, Ccl7, Ccna1,
Ccnd2, Cd2ap, Cd38, Cd4, Cd69, Cdkn1a, Cfb, Chka, Cntn6, Col3a1, Cxcl10, Cxcl11, Cxcl9,
Cxcr2, Cyp2j6, Defb1, Dll1, Dsc2, Dusp5, Dusp7, Dynlt1f, Dysf, Ece1, Edn1, Eif2ak2, Eif2b1,
Eif4enif1, Enpp2, Epb4.1, Etv4, F8, Faf1, Fas, Fasl, Fgf1, Flna, Foxo1, Ftl1, Fut4, Gadd45b,
Gbp2, Gch1, Gent1, Gls, Gm14446, Gm17296, Gmpr, Gpr161, Guk1, H2-Oa, Hbb-y, Hist2h2aa1,
Hs6st1, Hsp90aa1, Ido1, Ifi204, Ifi27, Ifi27l2a, Ifi27l2b, Ifi35, Ifi44, Ifitm1, Ifitm2, Ifitm3, Ifrd1,
Ikbkg, Il15, Il15ra, Il1rn, Il6, Inadl, Inppl1, Irf2, Irf7, Isg15, Isg20, Jak2, Jup, Kcna3, Kdelr2,
Kif20b, Klf6, Kpnb1, Krt8, Lag3, Lamp3, Lap3, Lepr, Lgals2, Lgals3bp, Lgals9, Lgmn, Lmnb1,
Lmo2, LOC102638047, Ly6e, Map2k5, Mcl1, Med1, Mgll, Mrps15, Msr1, Mx1, Mx2, Myd88,
Nampt, Nfe2l3, Nktr, Nmi, Nr3c1, Nub1, Nupr1, Oas1g, Oas2, Oas3, Pdgfb, Pdgfrl, Pkd2, Plscr1,
Pmaip1, Pml, Prkra, Psmb9, Ptch1, Rbck1, Rgs1, Rgs6, Rtp4, Sat1, Scarb2, Serping1, Sit1, Socs1,
Sp100, Sp140, Spib, St3gal5, Stap1, Stat1, Stat2, Stx11, Supt3, Tap1, Tap2, Tcn2, Tfdp2, Tgm1,
Tlr3, Tlr7, Tnfrsf11a, Tnfsf10, Tnk2, Tor1b, Tra2b, Trim21, Trim34a, Trim38, Uba7, Ube2l6,
Ube2s, Unc93b1, Usp18, Wars, Wt1, Xafl

### References (Each Incorporated by Reference Herein in its Entirety)

1. Deng, G.-M. & Tsokos, G. C. Pathogenesis and targeted treatment of skin injury in SLE. Nat. Rev. Rheumatol. 11, 663-669 (2015).
2. Bagavant, H. & Fu, S. M. Pathogenesis of kidney disease in systemic lupus erythematosus. Current Opinion in Rheumatology vol. 21 489-494 (2009).
3. Morel, L. Immunometabolism in systemic lupus erythematosus. Nat. Rev. Rheumatol. 13, 280-290 (2017).
4. Yin, Y. et al. Normalization of CD4+ T cell metabolism reverses lupus. Sci. Transl. Med. 7, 274ra18 (2015).
5. Li, W., Sivakumar, R., Titov, A. A., Choi, S.-C. & Morel, L. Metabolic Factors that Contribute to Lupus Pathogenesis. Crit. Rev. Immunol. 36, 75 (2016).
6. Reiss, A. B. Effects of inflammation on cholesterol metabolism: Impact on systemic lupus erythematosus. Curr. Rheumatol. Rep. 11, 255-260 (2009).
7. Feichtinger, R. G., Sperl, W., Bauer, J. W. & Kofler, B. Mitochondrial dysfunction: A neglected component of skin diseases. Exp. Dermatol. 23, 607-614 (2014).
8. Kuehne, A. et al. Acute Activation of Oxidative Pentose Phosphate Pathway as First-Line Response to Oxidative Stress in Human Skin Cells. Mol. Cell 59, 359-371 (2015).
9. Biniecka, M. et al. Dysregulated bioenergetics: a key regulator of joint inflammation. Ann. Rheum. Dis. 75, 2192-2200 (2016).
10. Adams, S. B. et al. Global metabolic profiling of human osteoarthritic synovium. Osteoarthr. Cartil. 20, 64-7 (2012).
11. Kang, H. M. et al. Defective fatty acid oxidation in renal tubular epithelial cells has a key role in kidney fibrosis development. Nat. Med. 21, 37-46 (2015).
12. Simon, N. & Hertig, A. Alteration of fatty acid oxidation in tubular epithelial cells: From acute kidney injury to renal fibrogenesis. Front. Med. 2, (2015).
13. Kelly, B. & O'Neill, L. A. Metabolic reprogramming in macrophages and dendritic cells in innate immunity. Cell Res. 25, 771-784 (2015).
14. MacIver, N. J., Michalek, R. D. & Rathmell, J. C. Metabolic Regulation of T Lymphocytes. Annu. Rev. Immunol. 31, 259-283 (2013).
15. Grayson, P. C. et al. Metabolic pathways and immunometabolism in rare kidney diseases. Ann. Rheum. Dis. 77, 1227-1234 (2018).
16. Tilstra, J. S. et al. Kidney-infiltrating T cells in murine lupus nephritis are metabolically and functionally exhausted. J. Clin. Invest. 128, 4884-4897 (2018) is incorporated by reference herein in its entirety.
17. Kidani, Y. & Bensinger, S. J. Lipids rule: resetting lipid metabolism restores T cell function in systemic lupus erythematosus. J. Clin. Invest. 124, 482-485 (2014) is incorporated by reference herein in its entirety.
18. Sharabi, A. & Tsokos, G. C. T cell metabolism: new insights in systemic lupus erythematosus pathogenesis and therapy. Nat. Rev. Rheumatol. 16, 100-112 (2020).
19. Mehta, M. M. & Chandel, N. S. Targeting metabolism for lupus therapy. Sci. Transl. Med. 7, 274fs5 (2015).
20. Banchereau, R. et al. Personalized Immunomonitoring Uncovers Molecular Networks that Stratify Lupus Patients. Cell 165, 551-565 (2016).
21. Catalina, M. D. et al. Patient ancestry significantly contributes to molecular heterogeneity of systemic lupus erythematosus. JCI Insight 5, (2020).
22. Hanzelmann, S., Castelo, R. & Guinney, J. GSVA: gene set variation analysis for microarray and RNA-Seq data. BMC Bioinformatics 14, 7 (2013).
23. Frankenberger, M., Schwaeble, W. & Ziegler-Heitbrock, L. Expression of M-Ficolin in human monocytes and macrophages. Mol. Immunol. 45, 1424-1430 (2008).
24. Xue, D., Tabib, T., Morse, C. & Lafyatis, R. Transcriptome landscape of myeloid cells in human skin reveals diversity, rare populations and putative DC progenitors. J. Dermatol. Sci. 97, 41-49 (2020).
25. Zimmerman, K. A. et al. Single-cell RNA sequencing identifies candidate renal resident macrophage gene expression signatures across species. J. Am. Soc. Nephrol. 30, 767-781 (2019).
26. Weening, J. J. et al. The classification of glomerulonephritis in systemic lupus erythematosus revisited. Kidney Int. 65, 521-530 (2004).
27. Agostinelli, C. Robust stepwise regression. J. Appl. Stat. 29, 825-840 (2002).
28. Krzywinski, M. & Altman, N. Classification and regression trees. Nature Methods vol. 14 757-758 (2017).
29. Afshinnia, F. et al. Impaired B-oxidation and altered complex lipid fatty acid partitioning with advancing CKD. J. Am. Soc. Nephrol. 29, 295-306 (2018).
30. Arazi, A. et al. The immune cell landscape in kidneys of patients with lupus nephritis. Nat. Immunol. 20, 902-914 (2019).
31. Wanders, R. J. A., Waterham, H. R. & Ferdinandusse, S. Metabolic interplay between peroxisomes and other subcellular organelles including mitochondria and the endoplasmic reticulum. Front. Cell Dev. Biol. 3, 83 (2016).
32. Shu, S. et al. Hypoxia and Hypoxia-Inducible Factors in Kidney Injury and Repair. Cells 8, 207 (2019).
33. Baechler, E. C. et al. Interferon-inducible gene expression signature in peripheral blood cells of patients with severe lupus. Proc. Natl. Acad. Sci. 100, 2610-2615 (2003).
34. Bennett, L. et al. Interferon and granulopoiesis signatures in systemic lupus erythematosus blood. J. Exp. Med. 197, 711-23 (2003).
35. Catalina, M. D., Bachali, P., Geraci, N. S., Grammer, A. C. & Lipsky, P. E. Gene expression analysis delineates the potential roles of multiple interferons in systemic lupus erythematosus. Commun. Biol. 2, 140 (2019).
36. Ahmed, D. et al. Transcriptional Profiling Suggests Extensive Metabolic Rewiring of Human and Mouse Macrophages during Early Interferon Alpha Responses. Mediators Inflamm. 2018, (2018).
37. Pantel, A. et al. Direct Type I IFN but Not MDA5/TLR3 Activation of Dendritic Cells Is Required for Maturation and Metabolic Shift to Glycolysis after Poly IC Stimulation. PLoS Biol. 12, (2014).
38. Wu, D. et al. Type 1 Interferons Induce Changes in Core Metabolism that Are Critical for Immune Function. Immunity 44, 1325-1336 (2016).
39. De Souza, D. P. et al. Autocrine IFN-I inhibits isocitrate dehydrogenase in the TCA cycle of LPS-stimulated macrophages. J. Clin. Invest. 129, 4239-4244 (2019).
40. Gardet, A. et al. Pristane-Accelerated Autoimmune Disease in (SWR X NZB) F1 Mice Leads to Prominent Tubulointerstitial Inflammation and Human Lupus Nephritis-Like Fibrosis. PLoS One 11, e0164423 (2016).
41. Bethunaickan, R. et al. Identification of stage-specific genes associated with lupus nephritis and response to remission induction in (NZB x NZW)F1 and NZM2410 mice. Arthritis Rheumatol. 66, 2246-2258 (2014).
42. Castillo-Rodriguez, E. et al. Kidney Injury Marker 1 and Neutrophil Gelatinase-Associated Lipocalin in Chronic Kidney Disease. Nephron 136, 263-267 (2017).
43. Viau, A. et al. Lipocalin 2 is essential for chronic kidney disease progression in mice and humans. J. Clin. Invest. 120, 4065-4076 (2010).
44. Viola, A., Munari, F., Sanchez-Rodriguez, R., Scolaro, T. & Castegna, A. The metabolic signature of macrophage responses. Front. Immunol. 10, 1462 (2019).
45. Waters, L. R., Ahsan, F. M., Wolf, D. M., Shirihai, O. & Teitell, M. A. Initial B Cell Activation Induces Metabolic Reprogramming and Mitochondrial Remodeling. iScience 5, 99-109 (2018).
46. Davidson, A. What is damaging the kidney in lupus nephritis?Nature Reviews Rheumatology vol. 12 143-153 (2016).
47. Schiffer, L. et al. Activated Renal Macrophages Are Markers of Disease Onset and Disease Remission in Lupus Nephritis. J. Immunol. 180, 1938-1947 (2008).
48. Wickersham, M., Wachtel, S., Fok, T. W., Richardson, A. & Parker, D. Metabolic Stress Drives Keratinocyte Defenses against Staphylococcus aureus Infection. Cell Reports 18, 2742-2751 (2017).
49. Wu, S.-B. & Wei, Y.-H. AMPK-mediated increase of glycolysis as an adaptive response to oxidative stress in human cells: Implication of the cell survival in mitochondrial diseases. Biochim. Biophys. Acta-Mol. Basis Dis. 1822, 233-247 (2012).
50. Zhao, X. et al. Metabolic regulation of dermal fibroblasts contributes to skin extracellular matrix homeostasis and fibrosis. Nat. Metab. 1, 147-157 (2019).
51. Hallan, S. et al. Metabolomics and Gene Expression Analysis Reveal Down-regulation of the Citric Acid (TCA) Cycle in Non-diabetic CKD Patients. EBioMedicine 26, 68-77 (2017).
52. Sharma, K. et al. Metabolomics Reveals Signature of Mitochondrial Dysfunction in Diabetic Kidney Disease. J. Am. Soc. Nephrol. 24, 1901-1912 (2013).
53. Berthier, C. C. et al. Cross-Species Transcriptional Network Analysis Defines Shared Inflammatory Responses in Murine and Human Lupus Nephritis. J. Immunol. 189, 988-1001 (2012).
54. Nakagawa, T., Kosugi, T., Haneda, M., Rivard, C. J. & Long, D. A. Abnormal angiogenesis in diabetic nephropathy. Diabetes 58, 1471-1478 (2009).
55. Ballermann, B. J. Glomerular endothelial cell differentiation. Kidney Int. 67, 1668-1671 (2005).
56. Sun, Y. B. Y. et al. Glomerular Endothelial Cell Injury and Damage Precedes That of Podocytes in Adriamycin-Induced Nephropathy. PLoS One 8, e55027 (2013).
57. Eelen, G., De Zeeuw, P., Simons, M. & Carmeliet, P. Endothelial cell metabolism in normal and diseased vasculature. Circ. Res. 116, 1231-1244 (2015).
58. Kalucka, J. et al. Quiescent Endothelial Cells Upregulate Fatty Acid β-Oxidation for Vasculoprotection via Redox Homeostasis. Cell Metab. 28, 881-894.e13 (2018).
59. Bhargava, P. & Schnellmann, R. G. Mitochondrial energetics in the kidney. Nature Reviews Nephrology vol. 13 629-646 (2017).
60. Eckardt, K. U. et al. Role of hypoxia in the pathogenesis of renal disease. in Kidney International vol. 68 S46-S51 (Elsevier, 2005).
61. Fritsch, S. D. & Weichhart, T. Effects of interferons and viruses on metabolism. Frontiers in Immunology vol. 7 630 (2016).
62. Broder, A. et al. Tubulointerstitial damage predicts end stage renal disease in lupus nephritis with preserved to moderately impaired renal function: A retrospective cohort study. Semin. Arthritis Rheum. 47, 545-551 (2018).
63. Chong, B. F. et al. A subset of CD163+ macrophages displays mixed polarizations in discoid lupus skin. Arthritis Res. Ther. 17, 1-10 (2015).
64. Katewa, A. et al. Btk-specific inhibition blocks pathogenic plasma cell signatures and myeloid cell-associated damage in IFNα-driven lupus nephritis. JCI insight 2, e90111 (2017).
65. Fu, J. et al. Transcriptomic analysis uncovers novel synergistic mechanisms in combination therapy for lupus nephritis. Kidney Int. 93, 416-429 (2018).
66. Labonte, A. C. et al. Identification of alterations in macrophage activation associated with disease activity in systemic lupus erythematosus. PLoS One 13, e0208132 (2018).
67. Daamen, A. R. et al. Comprehensive transcriptomic analysis of COVID-19 blood, lung, and airway. Sci. Rep. 11, 7052 (2021).
68. Dai, M. et al. Evolving gene/transcript definitions significantly alter the interpretation of GeneChip data. Nucleic Acids Res. 33, (2005).
69. Shannon, P. et al. Cytoscape: A software Environment for integrated models of biomolecular interaction networks. Genome Res. 13, 2498-2504 (2003).
70. Morris, J. H. et al. ClusterMaker: A multi-algorithm clustering plugin for Cytoscape. BMC Bioinformatics 12, 436 (2011).
71. Chockalingam, S., Aluru, M. & Aluru, S. Microarray Data Processing Techniques for Genome-Scale Network Inference from Large Public Repositories. Microarrays 5, 23 (2016).
72. Hubbard, E. L. et al. Analysis of gene expression from systemic lupus erythematosus synovium reveals myeloid cell-driven pathogenesis of lupus arthritis. Sci. Rep. 10, 17361 (2020).
73. Fagerberg, L. et al. Analysis of the human tissue-specific expression by genome-wide integration of transcriptomics and antibody-based proteomics. Mol. Cell. Proteomics 13, 397-406 (2014).
74. Uhlen, M. et al. Proteomics. Tissue-based map of the human proteome. Science 347(6220): 1260419-1260419 (2015).
75. Gazel, A. et al. Transcriptional Profiling of Epidermal Keratinocytes: Comparison of Genes Expressed in Skin, Cultured Keratinocytes, and Reconstituted Epidermis, Using Large DNA Microarrays. J. Invest. Dermatol. 121, 1459-1468 (2003).
76. Habuka, M. et al. The kidney transcriptome and proteome defined by transcriptomics and antibody-based profiling. PLoS One 9, (2014).
77. Franzen, O., Gan, L.-M. & Björkegren, J. L. M. PanglaoDB—A Single Cell Sequencing Resource For Gene Expression Data. panglaodb.se/index.html (2019).
78. Liberzon, A. et al. Molecular signatures database (MSigDB) 3.0. Bioinformatics 27, 1739-1740 (2011).
79. Subramanian, A. et al. Gene set enrichment analysis: A knowledge-based approach for interpreting genome-wide expression profiles. Proc. Natl. Acad. Sci. U.S.A 102, 15545-15550 (2005).
80. Kingsmore, K. M. et al. Transcriptomic Meta-analysis of Lupus Affected Tissues Reveals Shared Immune, Metabolic, and Biochemical Dysregulation [abstract]. Arthritis Rheumatol 71, (2019).

### Example 2: Machine Learning Reveals Distinct Gene Signature Profiles in Lesional and Nonlesional Regions of Inflammatory Skin Diseases

Inflammatory skin diseases have unique clinical features but may have both selective and overlapping responses to targeted therapies. To determine the unique and shared molecular features of inflammatory skin diseases, we carried out a comprehensive analysis of gene expression from cutaneous lupus erythematosus (CLE) and compared it to that of psoriasis, atopic dermatitis, and systemic sclerosis. Using gene set variation analysis (GSVA), we found that lesional samples from each condition had unique features, but all four diseases displayed common enrichment in multiple inflammatory cell and pathway gene signatures, including the interferon, tumor necrosis factor, and IL-23 gene signatures. These findings were confirmed by both classification and regression tree (CART) analysis and machine learning (ML) models. Nonlesional samples from each disease also differed from normal samples and each other by ML. Notably, the features used in classification of nonlesional disease compared to control were more distinct than their lesional counterparts and GSVA confirmed unique features of nonlesional disease. These data show that lesional and nonlesional skin samples from CLE and other inflammatory skin diseases have unique profiles of gene expression abnormalities, especially in nonlesional skin. The results suggest a model in which diseases-specific abnormalities in “pre-lesional” skin may permit environmental stimuli to trigger inflammatory responses leading to both the unique and shared manifestations of each disease. Dissection of molecular pathways enriched in both clinically involved and uninvolved skin can advance the understanding of the pathogenesis of these conditions and identify novel therapies.

Autoimmune and inflammatory diseases, such as systemic lupus erythematosus (SLE), can affect many organs, including the skin. Indeed, skin manifestations of lupus, known as cutaneous lupus erythematosus (CLE), are common and occur in 70-85% of lupus patients (1-3). Historically, CLE is classified into three subtypes based on clinical and serological features: acute CLE (ACLE), subacute CLE (SCLE), and chronic CLE (CCLE) (4). The heterogeneity of CLE makes it difficult to determine particular cytokines or inflammatory pathways to target therapeutically and, as a result, no therapies are specifically approved for CLE (5). Both an innate immune response, coordinated through Toll-like receptor activation as well as multiple adaptive immune responses have been reported in the initiation and propagation of CLE (2). Targeting B cells with belimumab (6) and type 1 interferon (IFN) with anifrolumab (7) show some benefit by decreasing cutaneous manifestations of SLE. In contrast, other inflammatory skin diseases, such as psoriasis (PSO) have numerous approved therapies (8), and dupilumab, an inhibitor of IL-4 receptor signaling, is an effective therapy for both atopic dermatitis (AD) and PSO (9). This overlap of central, nonredundant pathways between PSO and AD illustrates that diseases with markedly different clinical phenotypes may have similar immunopathogenic underpinnings.

Although independent transcriptomic analyses have provided insight into the molecular landscape of CLE, a complete molecular characterization of the disease is limited by small patient cohorts (10-17). Previous bulk gene expression studies focused on specific aspects of lupus skin disease, such as the presence of T helper (Th) 17 cells (10) or specific macrophage populations (11), the correlation between inflammatory cell populations and fibroblast marker expression (12), cytokine expression (13), inflammasome signaling (14) or IFN signaling (15-17). However, there remains a need to examine the interplay of inflammatory cells, non-hematopoietic cells, and pathway perturbations to understand the molecular events in CLE pathogenesis in further detail

Whereas the dissimilarities in clinical manifestations of CLE and other inflammatory skin diseases have been well documented, the molecular differences between CLE and other inflammatory skin diseases are less completely studied. For instance, keratinocytes, one of the predominant non-hematopoietic cell populations in the skin, have been implicated in PSO pathogenesis (18) and shown to be hypersensitive to IFN signaling in CLE (15), yet understanding of their role in CLE is limited. Moreover, systemic sclerosis (SSc), another inflammatory skin disease, is characterized by fibrosis and vascular damage due to excessive deposits of extracellular matrix and differentiation of fibroblasts to myofibroblasts (19), but little is known about the role of fibrosis in the pathogenesis of CLE. Finally, AD is characterized by an allergic reaction owing to a loss in skin barrier function, fibrosis, and Th2 cell signaling (20), but these functions in CLE have not been explored. Detailed comparison among the molecular signatures of CLE, PSO, AD, and SSc could achieve better understanding of the primary pathogenic mechanisms and provide direction for new therapeutic avenues in these conditions.

In this study, we compared the gene expression signatures of four inflammatory skin diseases: CLE, PSO, AD, and SSc. In order to achieve a read depth sufficient to maintain the in vivo proportions of cellular signals in the biopsies without technical distortion and to capture the majority of molecular pathways, we analyzed bulk RNA. In addition, we employed analytic tools to deconvolute transcriptomic data and determine cellular and pathway signals enriched across heterogeneous cohorts of patients from each of the diseases. Using gene set variation analysis (GSVA), we determined that lesional skin of the four skin diseases expressed both shared and unique molecular signatures. Machine learning (ML) demonstrated that both lesional and nonlesional samples of each disease could be classified as distinct from control samples as well as from each other. Notably, nonlesional skin of each disease was more distinct than lesional skin, as there were more common features in ML classification of lesional skin among the four diseases. GSVA confirmed the molecular differences between uninvolved skin of the various conditions. These results suggest a model in which the nonlesional skin of patients with inflammatory skin diseases harbors unique abnormalities that potentially make the skin differentially sensitive to specific environmental stimuli. Inciting stimuli appear to induce responses with many overlapping inflammatory molecular features shared by the diseases. Altogether, this suggests that therapeutics employed in the treatment of one inflammatory skin disease may be useful in the treatment of additional diseases and confirms the utility of gene expression analysis in understanding the immunopathogenesis of clinical and pre-clinical disease.

### Results

Comprehensive Gene Expression Analysis of DLE Reveals Similarities and Differences with Other Inflammatory Skin Diseases.

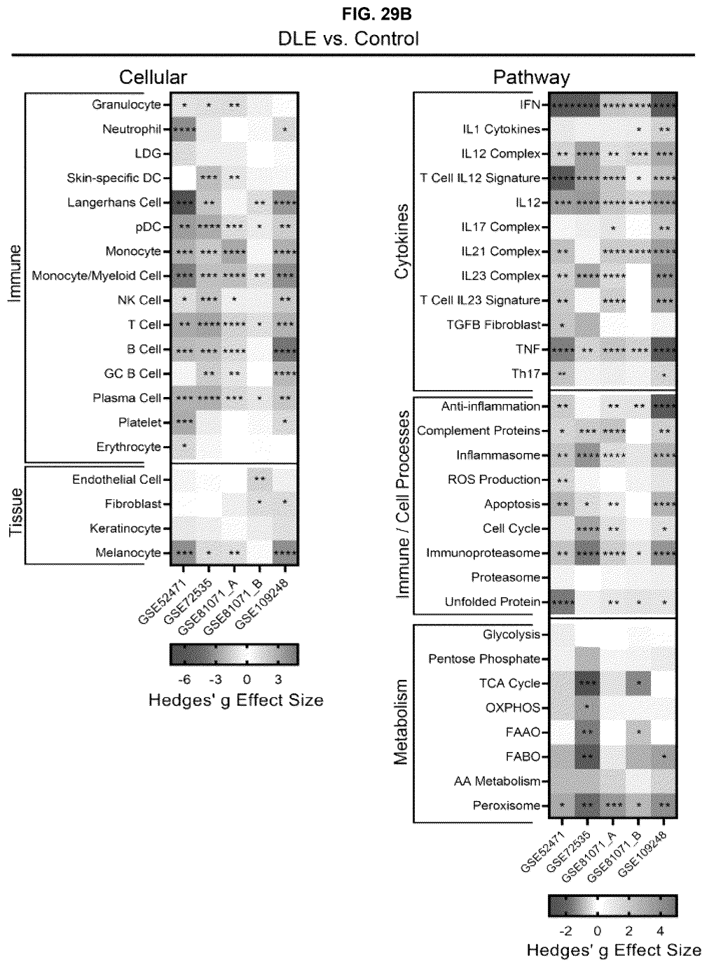

We carried out a comprehensive transcriptomic analysis of five independent datasets of samples biopsied from both patients with DLE, the most frequent subset of CLE (4), and healthy controls (Table 3). In order to examine cellular and pathway signaling on an individual patient level, we carried out GSVA using a total of 48 informative gene signatures (Table 4A-1 to 4A-20, and 4B-1 to 4B-28). Hierarchical clustering of GSVA enrichment scores demonstrated that DLE was molecularly separable from healthy skin (FIG. 29A). Despite some interpatient heterogeneity in each dataset, signatures for plasmacytoid dendritic cells (pDCs), monocytes, monocyte/myeloid cells, natural killer (NK) cells, T cells, B cells and plasma cells were consistently enriched in patients with DLE as compared to healthy skin (left, FIG. 29B). Conversely, signatures representative of granulocytes, Langerhans cells and melanocytes were consistently downregulated in lupus-affected skin. As previously shown, the interferon gene signature (IGS) was increased in all DLE datasets, as were the IL-12 and TNF gene signatures (14, 17) (right, FIG. 29B). There was also enrichment of gene expression of other inflammatory pathways, such as IL-21, IL-23, complement and the immunoproteasome. Additionally, genes reflective of most metabolic processes were decreased in DLE samples whereas signatures representing glycolysis and the pentose phosphate pathway remained mostly unchanged.

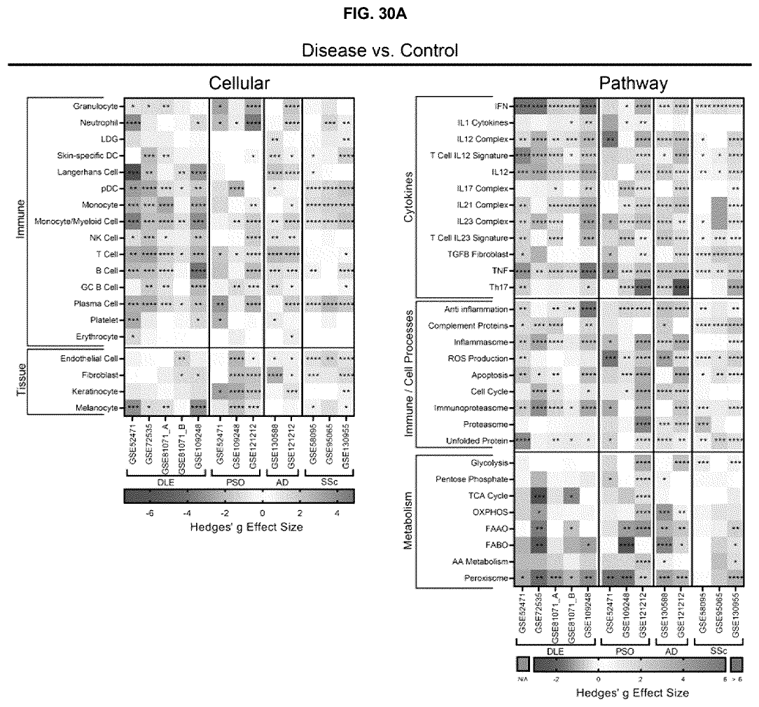

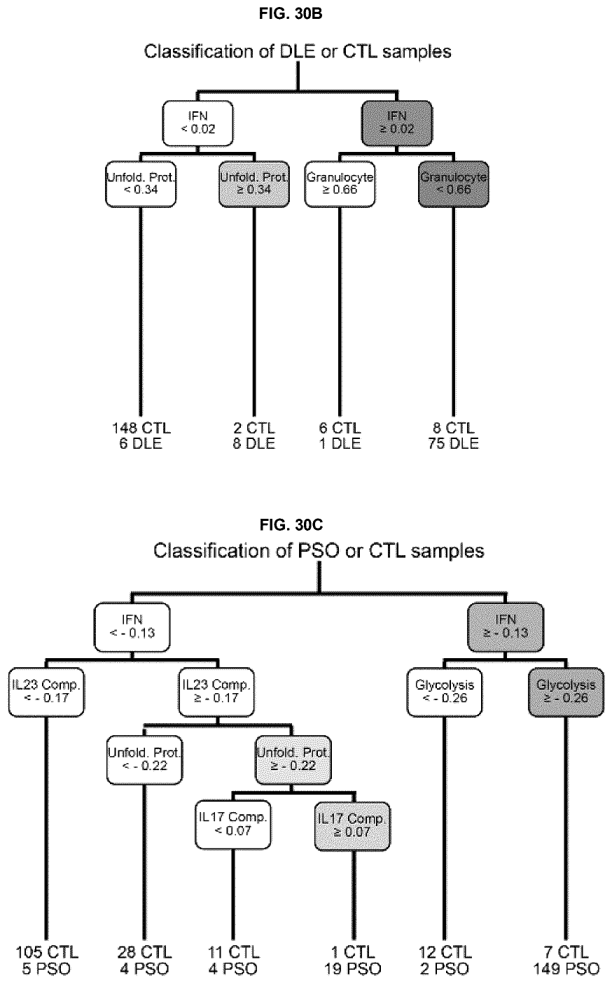

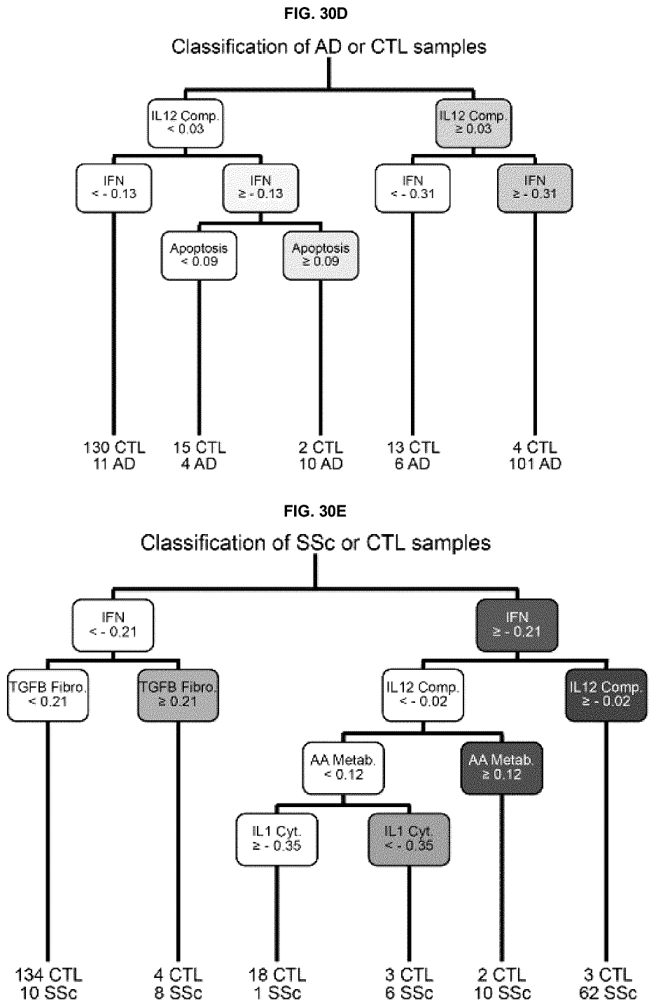

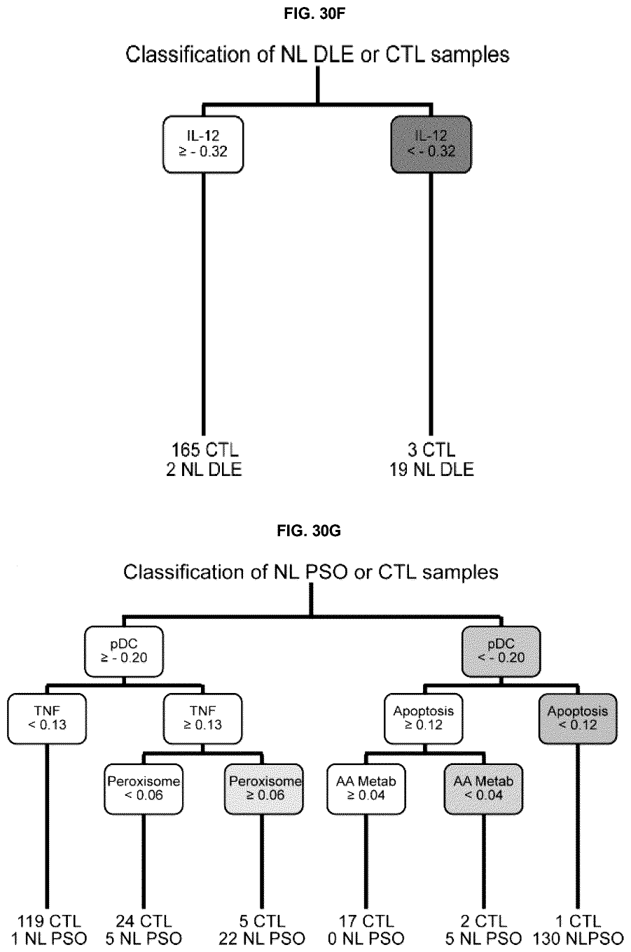

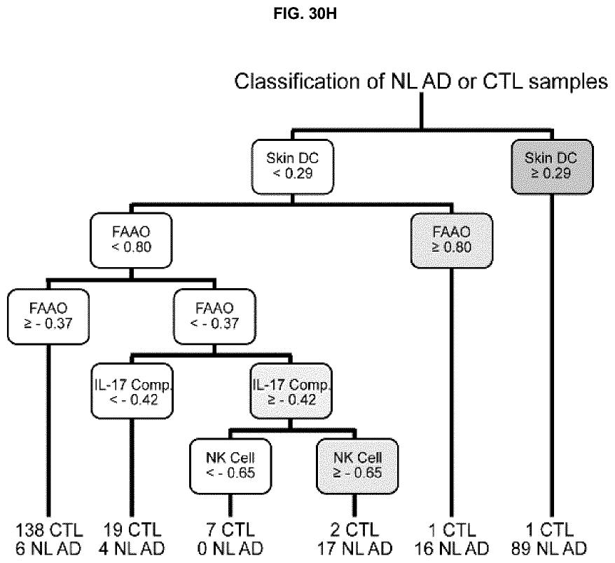

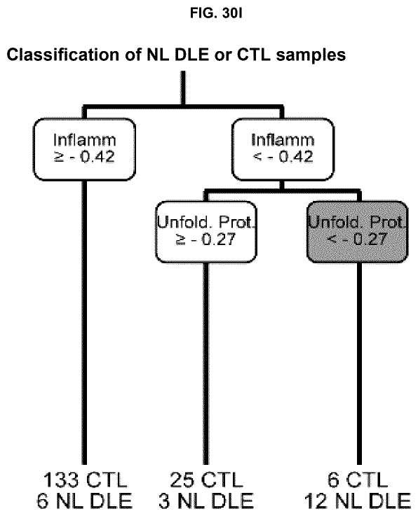

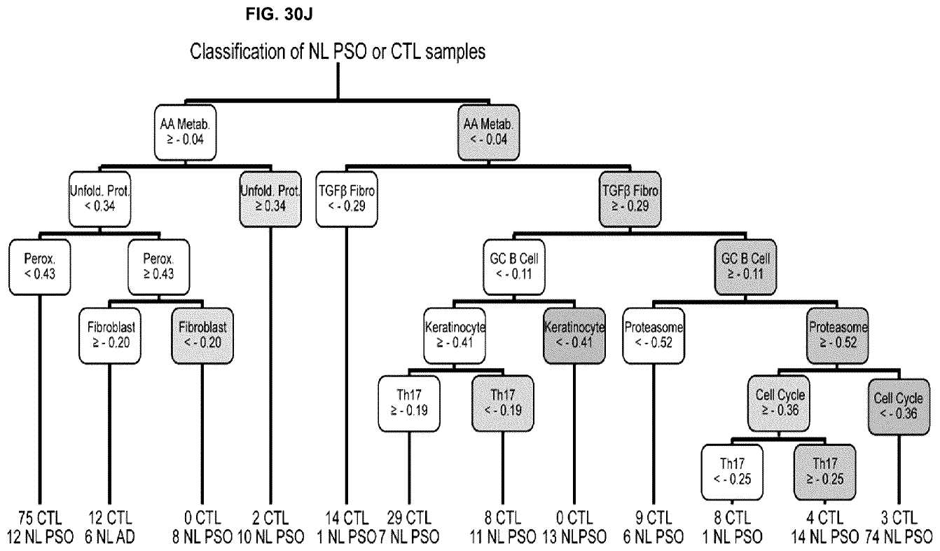

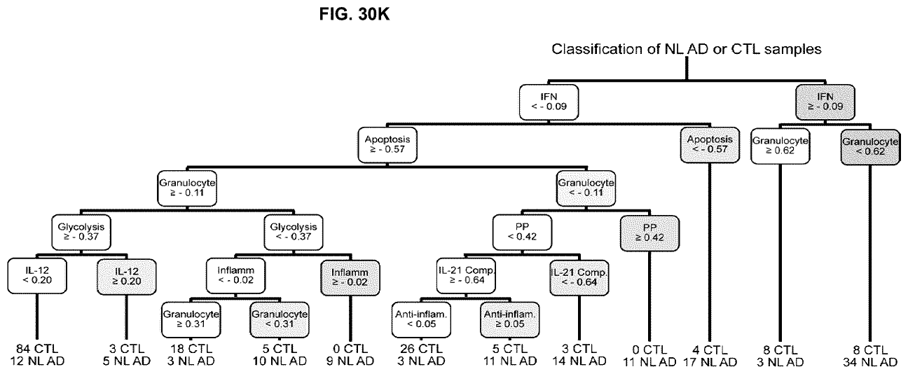

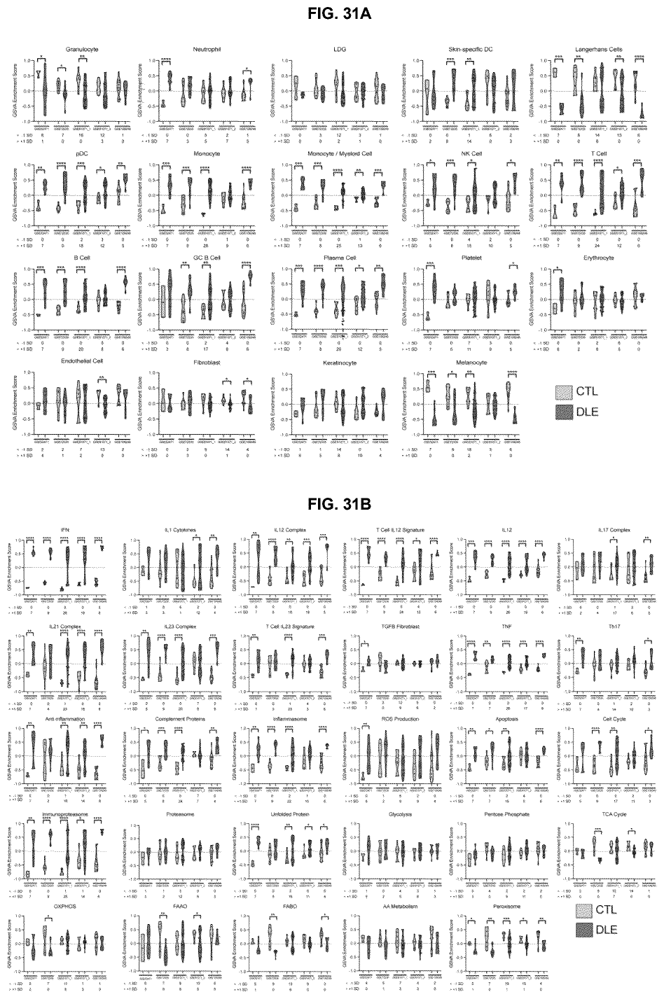

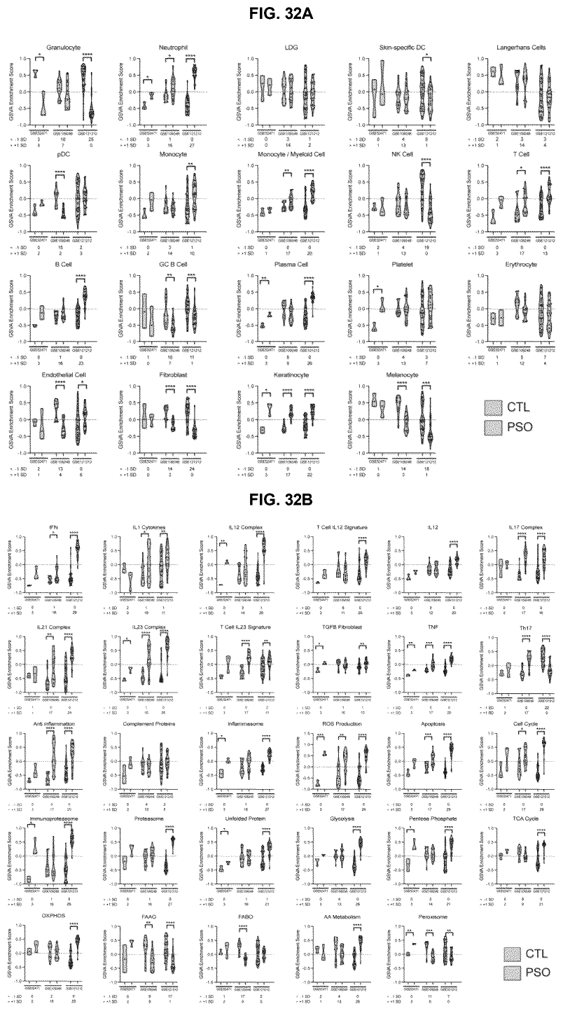

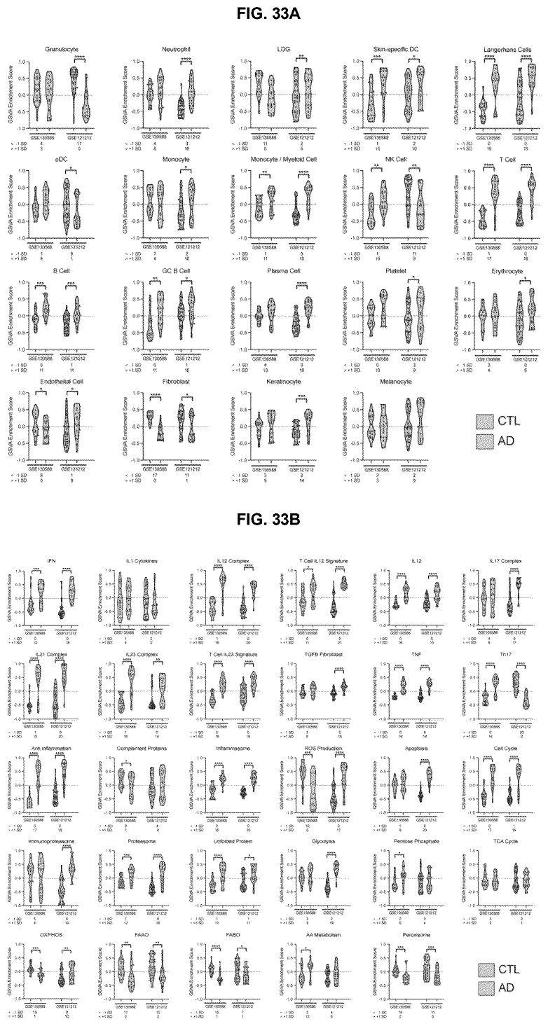

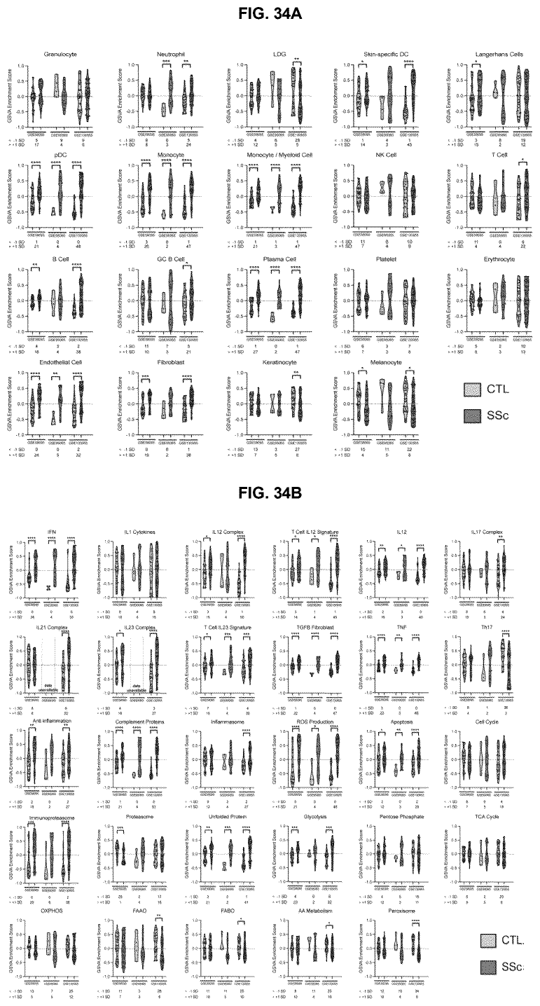

To understand the molecular landscape of cutaneous lupus in the context of other inflammatory skin diseases, we examined gene expression data derived from skin biopsies of patients with PSO, AD or SSc. Overall, there was enrichment of most myeloid and lymphoid-derived cell signatures across all four diseases as compared to control, whereas expression of skin-specific dendritic cells (DCs) differed among the diseases (left, FIG. 30A, FIG. 31-34). Monocyte gene signatures were consistently enriched in DLE, AD and SSc. In contrast, the endothelial cell gene signature was enriched in SSc only. Inflammatory cytokine gene expression was largely enriched in all four diseases, especially pathways involving IFN, IL-12, IL-23 and TNF. DLE and SSc exhibited gene expression enrichment in the complement signature, whereas PSO and AD did not (right, FIG. 30A, FIG. 31-34). We noted heterogeneity among datasets of each disease; for example, the Th17 signature was upregulated in two of three PSO datasets and the peroxisome signature was downregulated in four out of five DLE datasets.

Next, we employed classification and regression tree (CART) analysis using GSVA enrichment scores of 48 cellular and pathway gene signatures to discern the gene expression variables that best classified the inflammatory skin diseases (FIG. 30B-H). CART analyses four lesional (L) diseases (DLE, PSO, and SSc) were compared to controls; whereas, classification of nonlesional (NL) samples as compared to controls is dependent on different features in each nonlesional disease. The IGS, unfolded protein and granulocyte gene signatures were the most important signatures for classifying control or DLE (FIG. 30B), whereas the IGS and glycolysis signatures together classified 83% of PSO samples (FIG. 30C). In contrast, the IL-12 complex, IGS and apoptosis signatures classified AD or control (FIG. 30D), and the IGS, TGFB fibroblast, IL-12 complex, amino acid metabolism and IL-1 cytokine signatures were the most important features in classifying SSc or control (FIG. 30E). These data show that both common and disease-specific molecular pathway signatures classify the involved skin of the different inflammatory skin diseases.

### ML Classification of Lesional Inflammatory Skin Samples Confirms Unique and Common Molecular Pathways

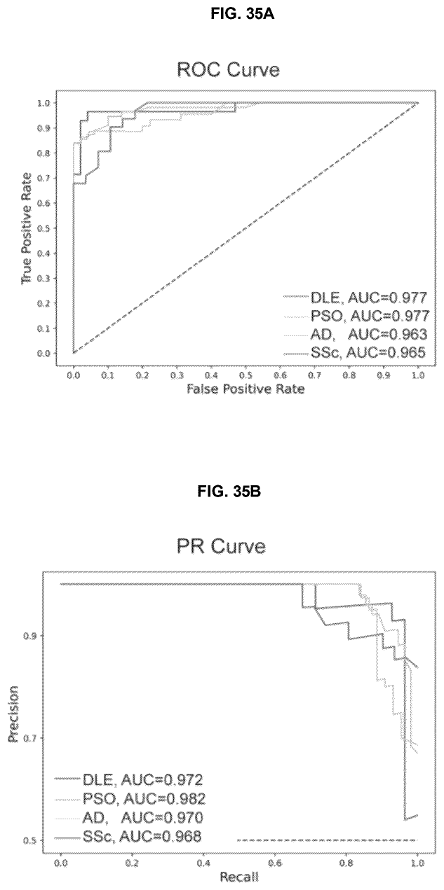

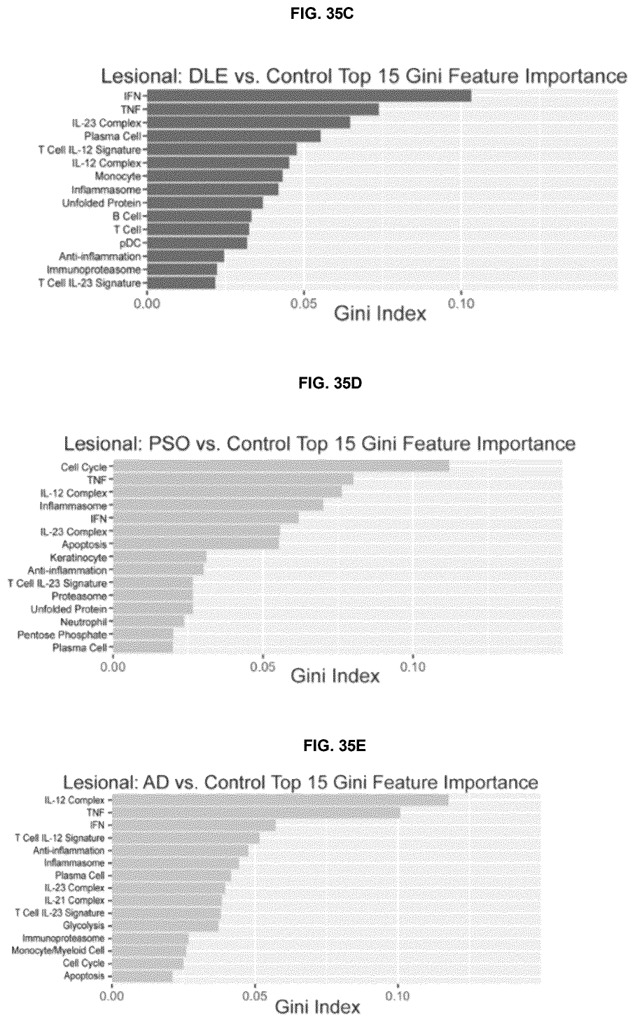

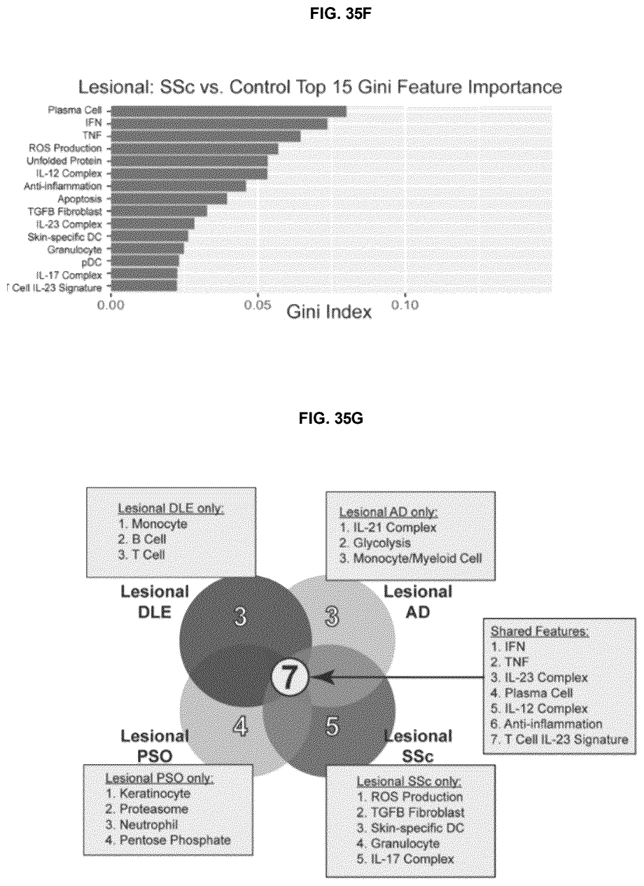

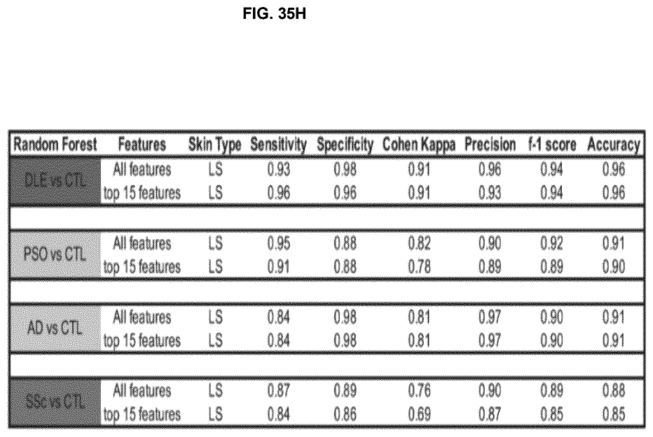

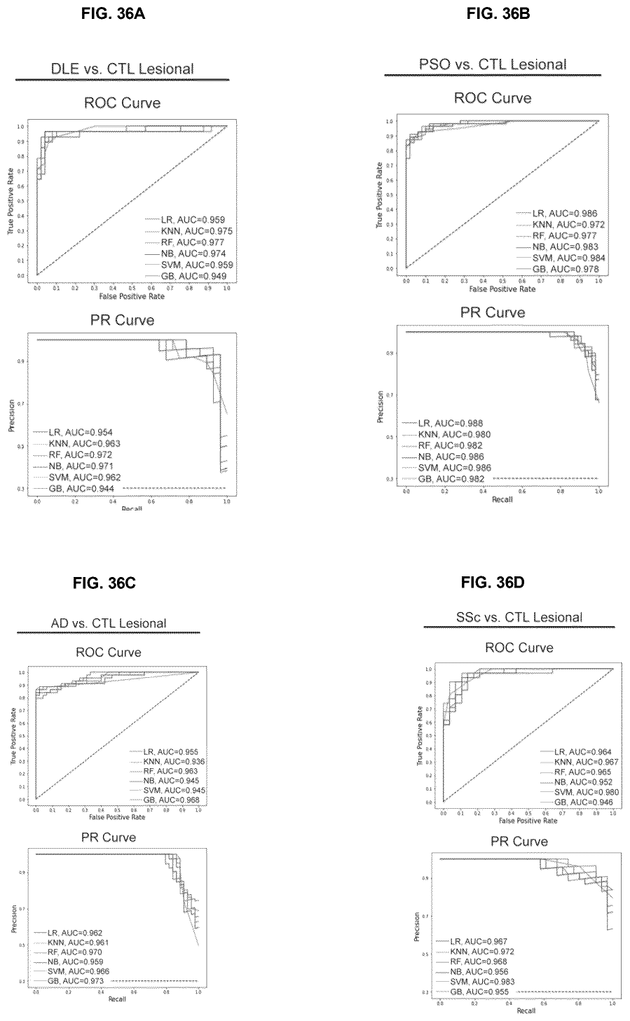

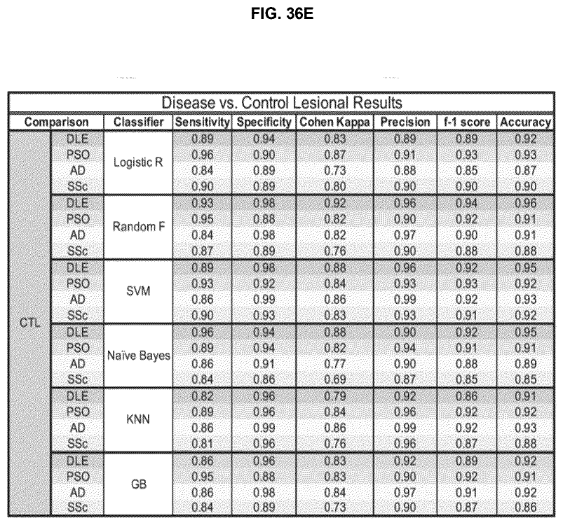

To distinguish inflammatory skin diseases more precisely and confirm the major transcriptomic contributors, we employed several ML algorithms. First, we examined distinct binary classification of pooled lesional DLE, PSO, AD, and SSc compared to pooled control samples using the ensemble decision tree, random forest (RF), with the 48 cellular and pathway signature GSVA scores as input features. The areas under the receiver operating characteristic (AuROC) curves and precision-recall (AuPR) curves for each binary classification were greater than 0.96 in all cases, indicating excellent performance and appropriate binary classification for each disease compared to control samples (FIG. 35A, B). For each binary comparison, we determined the top 15 most important features in separating disease from control samples using RF Gini feature importance. To classify DLE from control samples, the IGS, TNF and IL-23 complex signatures were the most important features (FIG. 35C), whereas to classify PSO from control samples, the cell cycle, TNF and IL-12 complex signatures were the most important features (FIG. 35D). To classify AD from control samples, the IL-12 complex and TNF signatures as well as the IGS were the most important features (FIG. 35E), and the plasma cell signature, IGS and TNF signature were the most important features to classify SSc (FIG. 35F). ML analysis elucidated seven features in common among the 15 most important features for classifying each inflammatory skin disease from control, including the IGS and the TNF, IL-23 complex, plasma cell, IL-12 complex, anti-inflammation and T cell IL-23 signatures (FIG. 35G). It is notable that the 15 most important features performed comparably to the full ensemble of 48 features in binary classification of inflammatory skin disease samples (FIG. 35H). Additionally, a number of other ML algorithms were similarly effective at binary classification of these samples (FIG. 36).

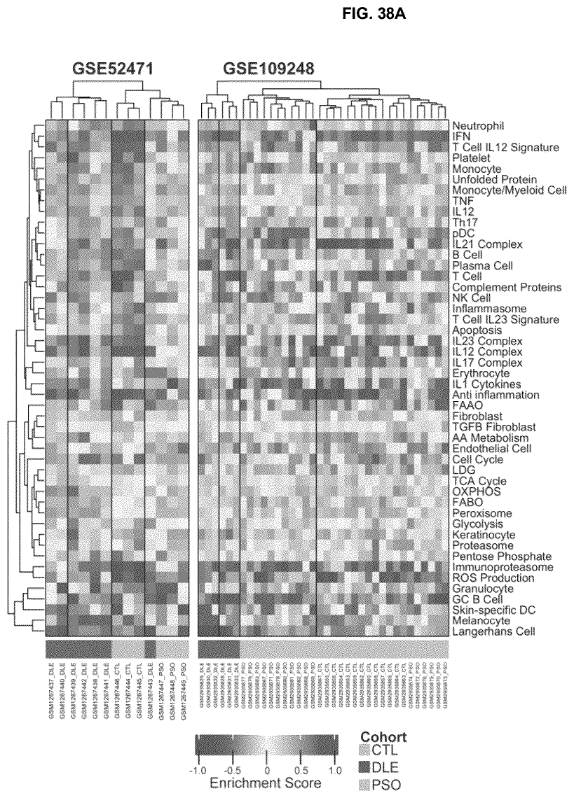

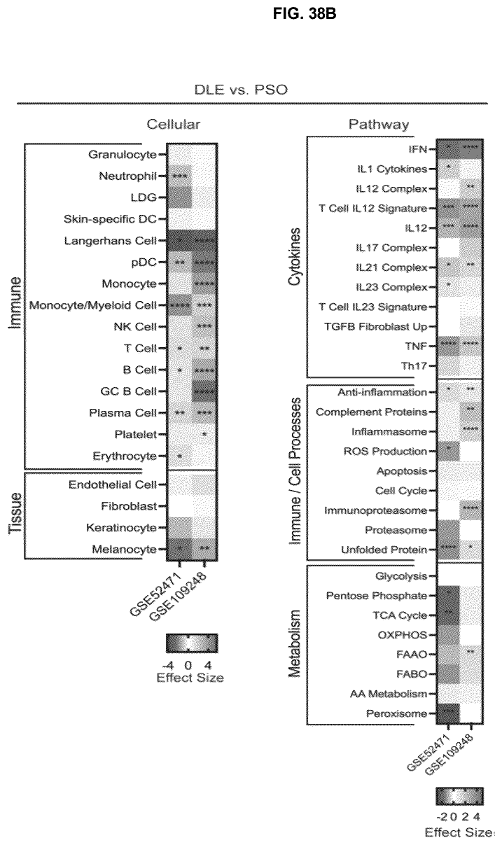

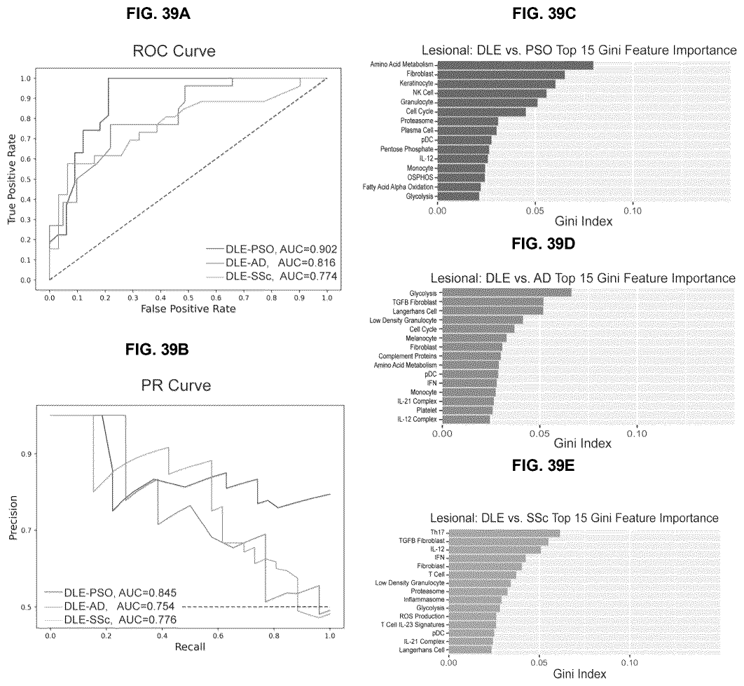

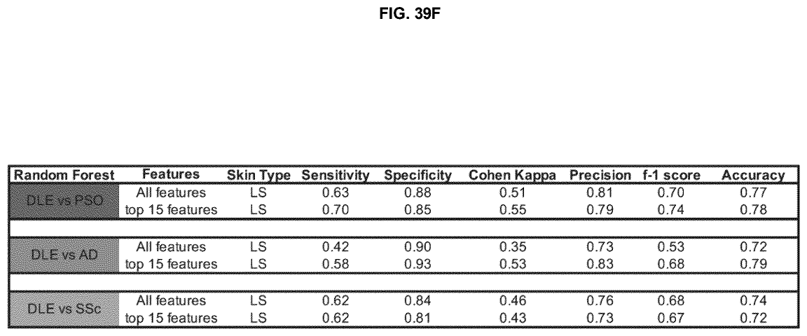

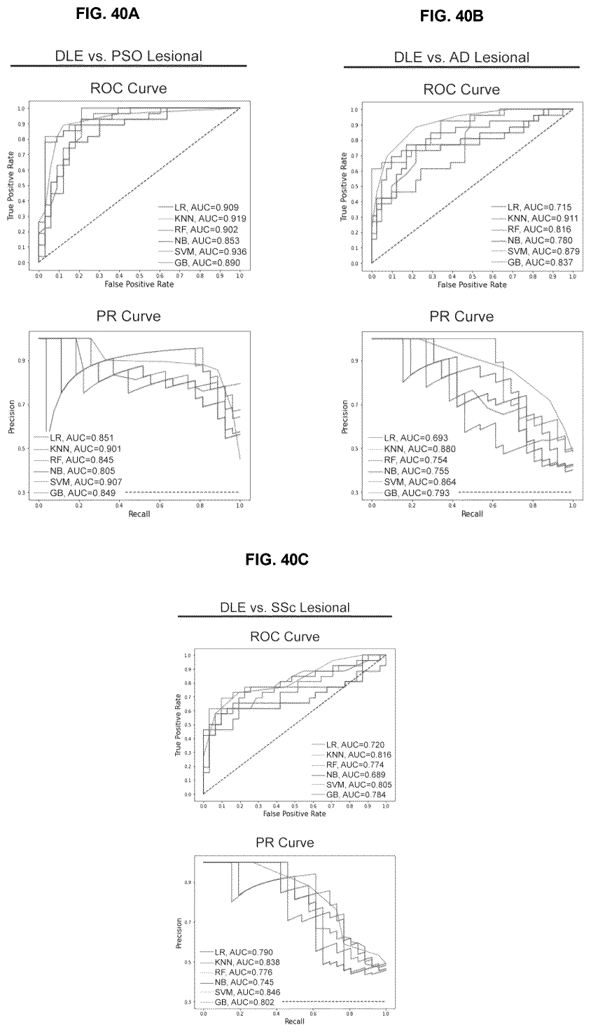

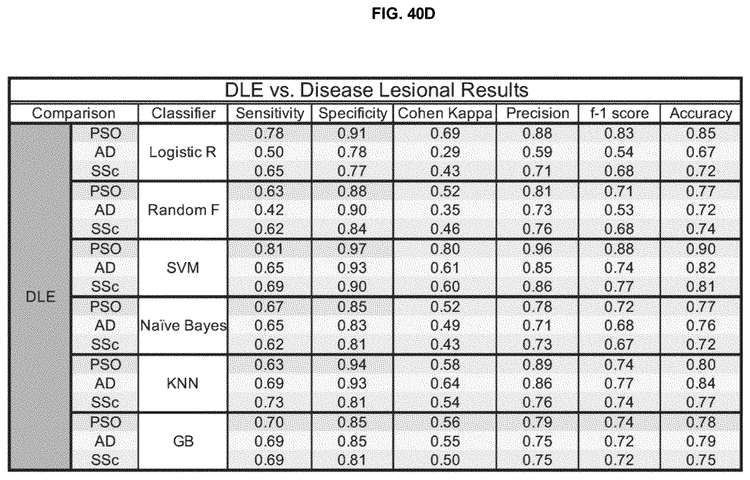

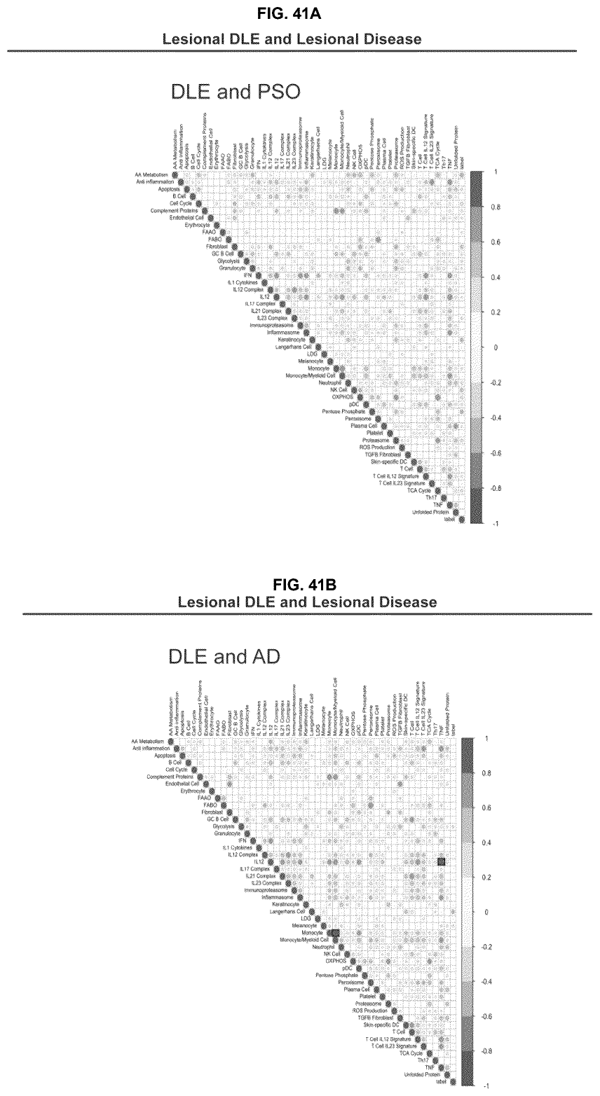

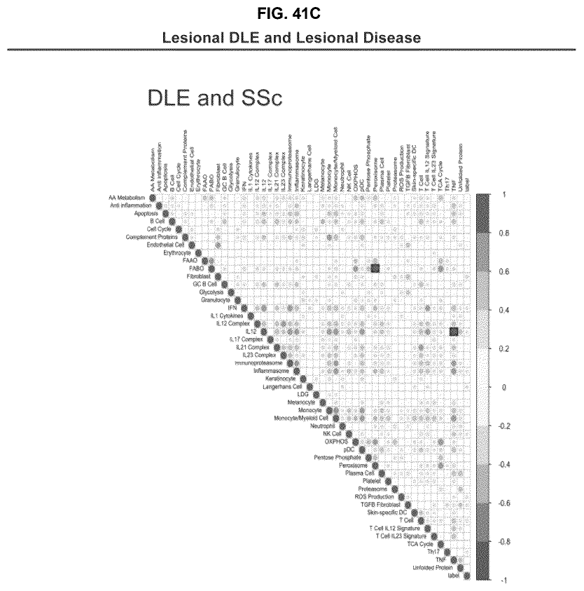

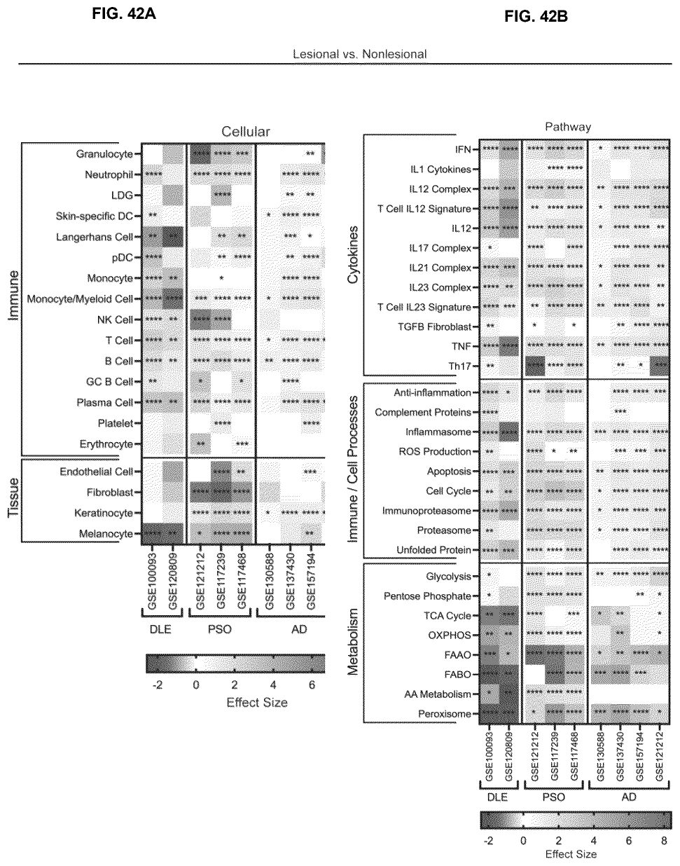

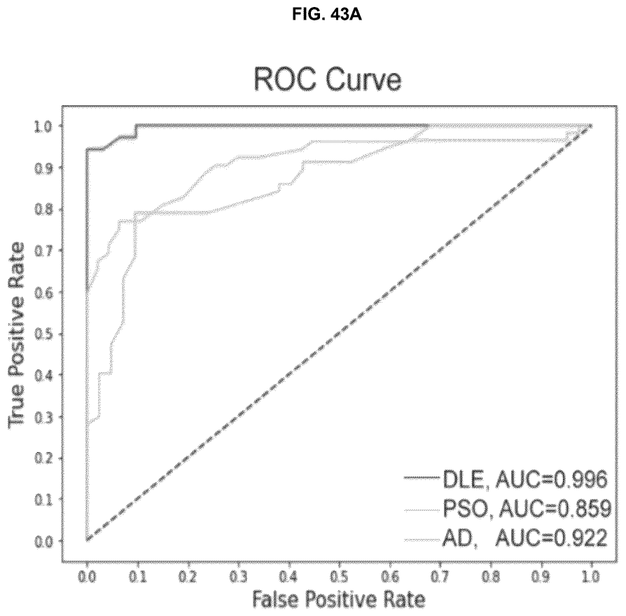

Next, we directly compared gene expression signatures of DLE samples with those of other inflammatory skin diseases. Distinctions in cellular and pathway signature enrichment among DLE and PSO samples were observed using hierarchical clustering of GSVA scores in two datasets with samples from these inflammatory skin conditions (FIG. 38). Notably, transcriptomic signatures that differed between DLE and PSO included the IGS as well as the Langerhans cell and T cell IL-12 gene signatures. Since we lacked datasets in which DLE samples were analyzed concurrently with AD and SSc, we employed ML algorithms to classify samples from these conditions using the 48 GSVA enrichment scores as features. Performance characteristics and AuROC and AuPR curves demonstrated less effective classification of DLE from other diseases than the classification of each disease versus control (FIG. 39 A, B). In the binary classification of DLE and PSO samples, the most important features included the amino acid metabolism, fibroblast and keratinocyte gene signatures (FIG. 39C), whereas classification of DLE and AD samples involved the glycolysis, TGFβ fibroblast and Langerhans cell gene signatures (FIG. 39D). The Th17, TGFβ fibroblast and IL-12 signatures were most important in separating DLE and SSc samples (FIG. 39E). Classification using only the top 15 most important features was as effective as when all 48 features were employed (FIG. 39F, Table 9). Finally, other ML classifiers performed similarly to RF (FIG. 40).

Transcriptomic Profiles of Nonlesional Skin Samples Distinguish Inflammatory Skin Diseases from Each Other

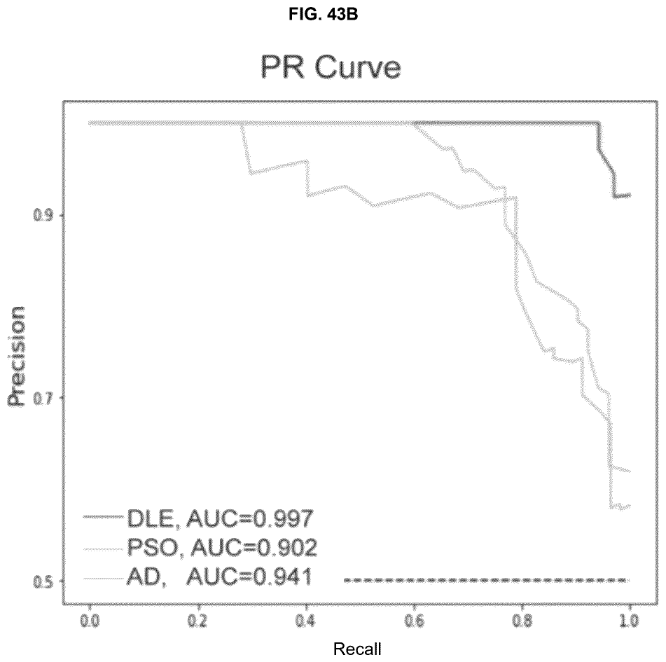

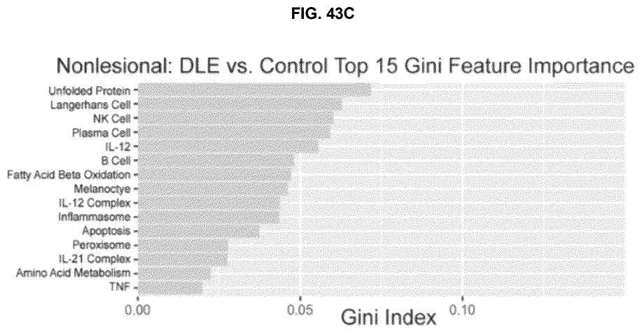

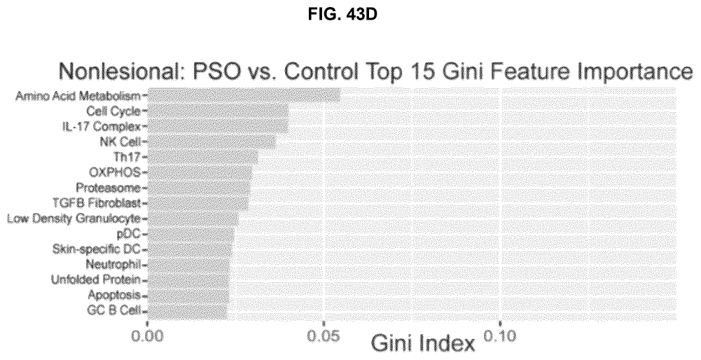

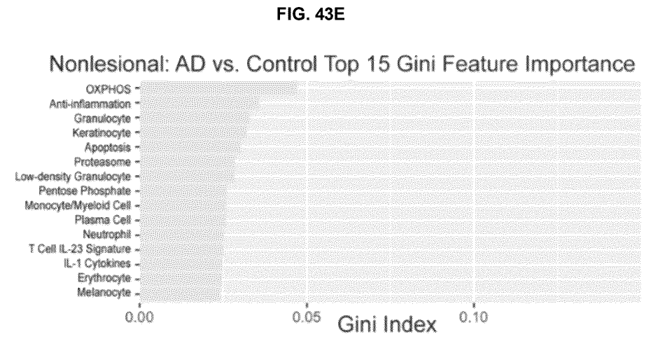

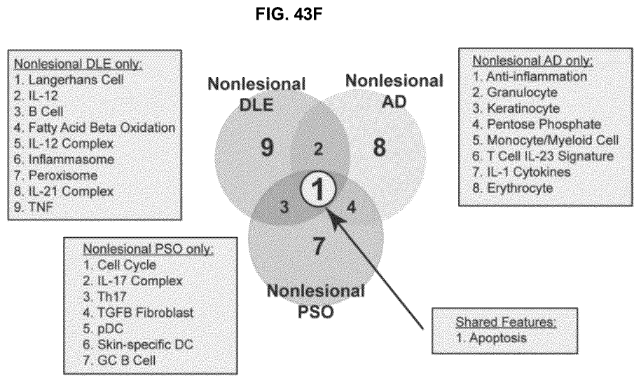

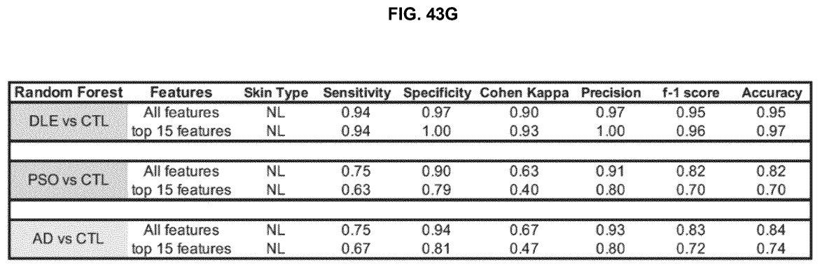

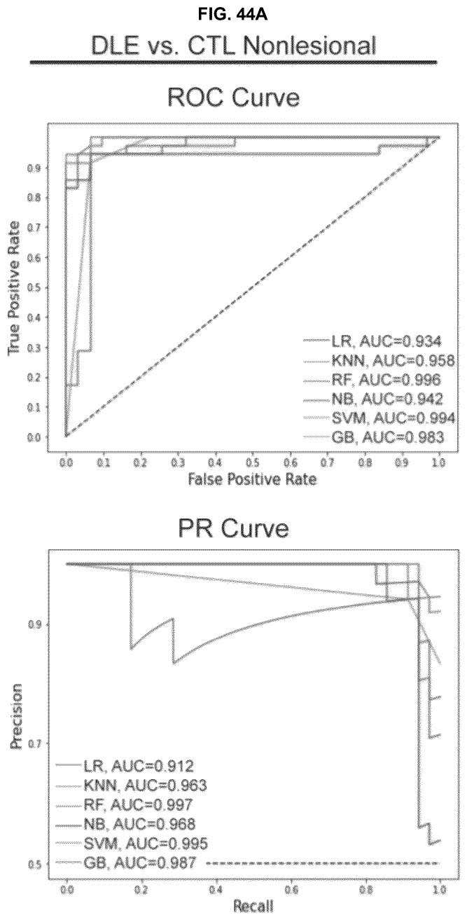

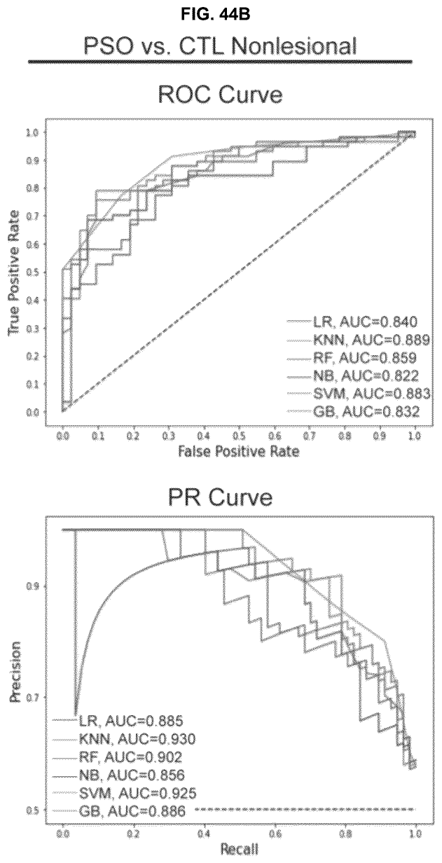

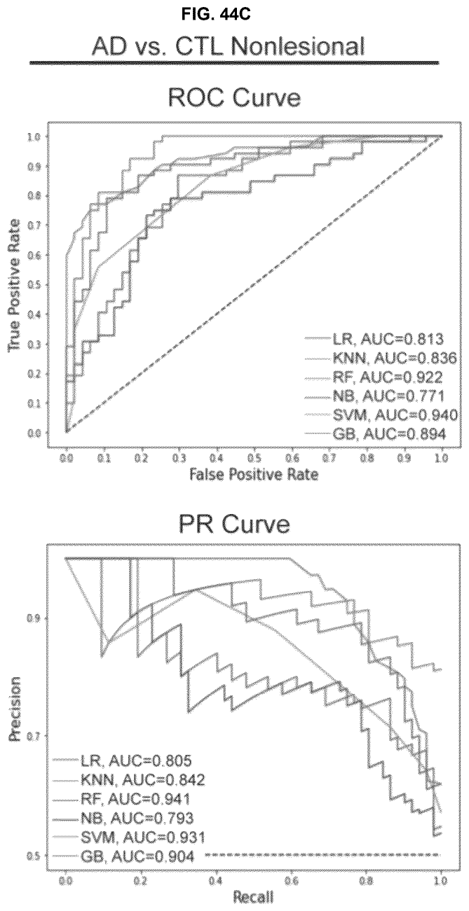

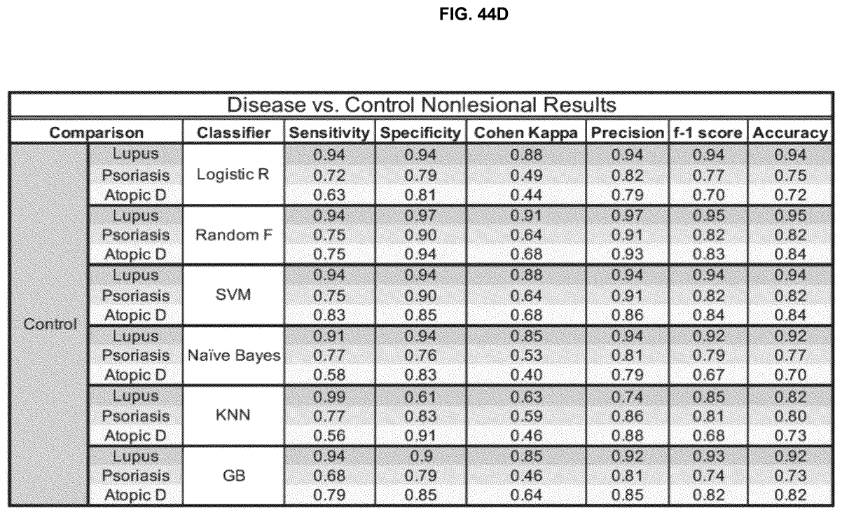

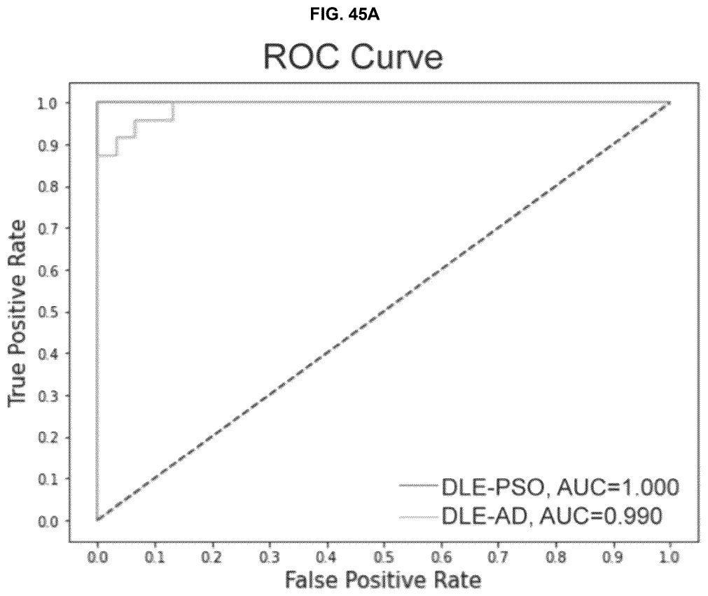

Although the molecular characteristics of lesional skin in each independent disease have been well-studied, less is known about the transcriptomic profiles of uninvolved skin. To determine whether there are underlying immunological abnormalities contributing to disease, we examined gene expression profiles in nonlesional skin samples from patients with DLE, PSO and AD to assess the extent to which they differed from either control or lesional skin; nonlesional SSc data was not available for analysis. Analysis of GSVA enrichment scores demonstrated that nonlesional samples were transcriptionally different from lesional samples (FIG. 42). In fact, the upregulation of inflammatory pathways including IFN, IL-12, IL-23 and TNF gene signatures in lesional versus nonlesional skin mirrored the upregulation of these same pathways observed in comparison of lesional versus control skin in DLE, PSO and AD (FIG. 30A). We next carried out ML classification using GSVA scores to determine whether nonlesional samples were different than control. To overcome class imbalance issues, we employed undersampling and synthetic minority oversampling technique (SMOTE) to balance the number of samples in each class to optimize each binary classification. ML was able to classify nonlesional disease and healthy control samples reliably (FIG. 43A, B). Classification of nonlesional DLE and control showed that the unfolded protein, Langerhans cell and NK cell signatures were the most important features (FIG. 43C), whereas the amino acid metabolism, cell cycle and IL-17 complex signatures were the most important features to classify nonlesional PSO and control (FIG. 43D). By contrast, the oxidative phosphorylation, anti-inflammation and granulocyte signatures were the most important features in classifying nonlesional AD from control (FIG. 43E). Notably, comparison of the top 15 features of each binary classification showed that there was minimal overlap of important features among nonlesional skin diseases, with only one feature, apoptosis, shared among nonlesional DLE, PSO and AD (FIG. 43F). The binary classifications performed accurately when only the top 15 features of each binary classification were employed (FIG. 43G, Table 11). Moreover, other ML classifiers performed similarly to RF (FIG. 44). These data indicate that nonlesional skin in each of the three diseases evaluated is uniquely different from control skin.

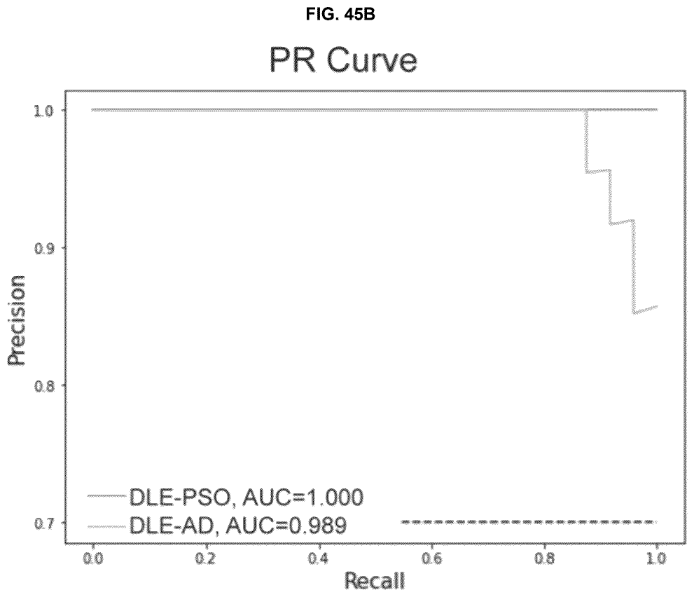

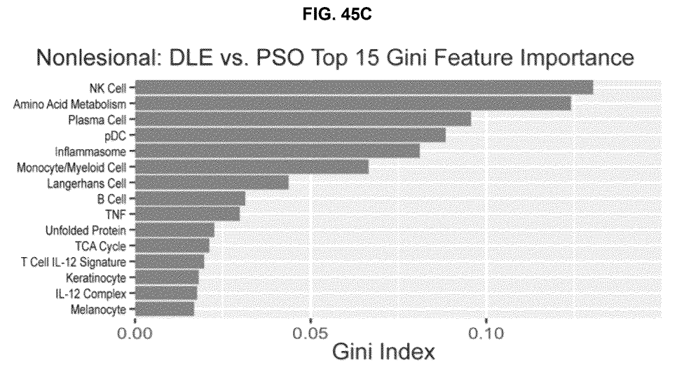

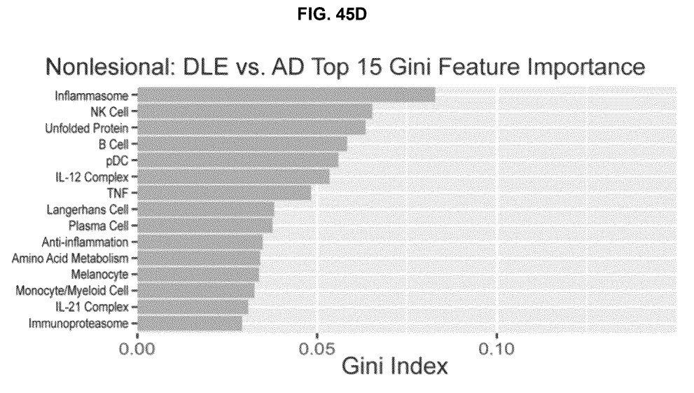

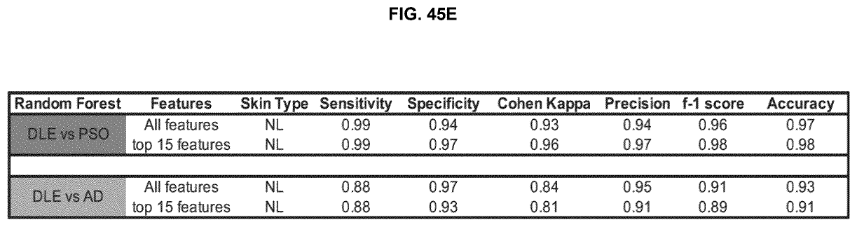

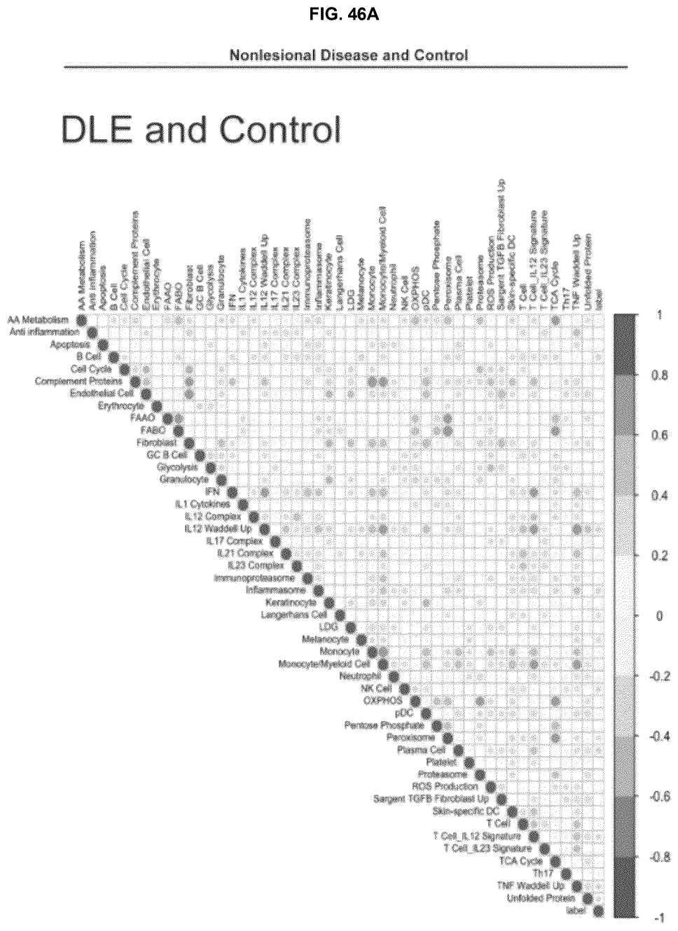

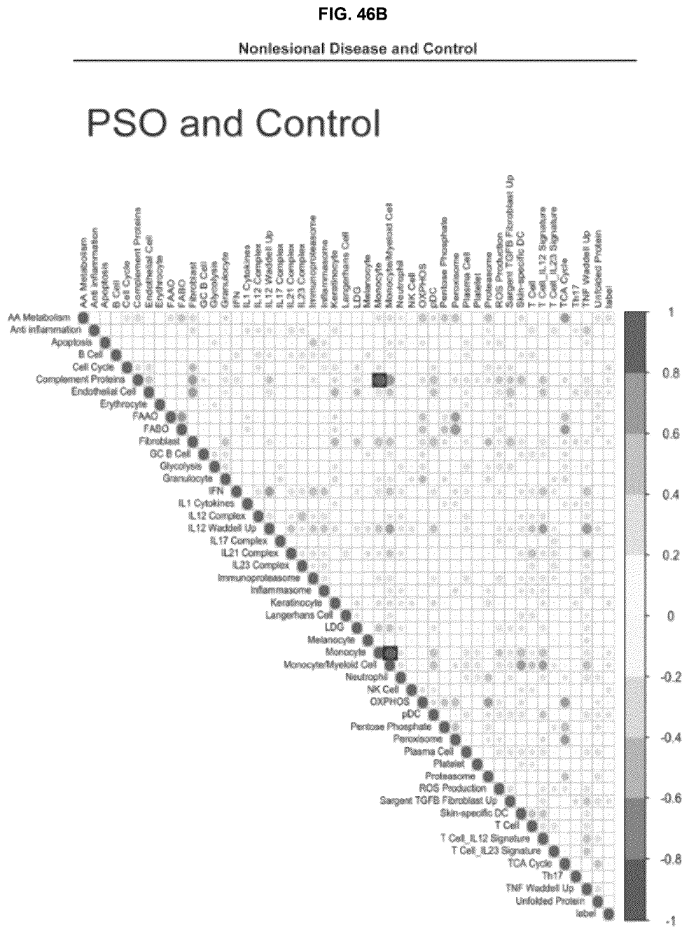

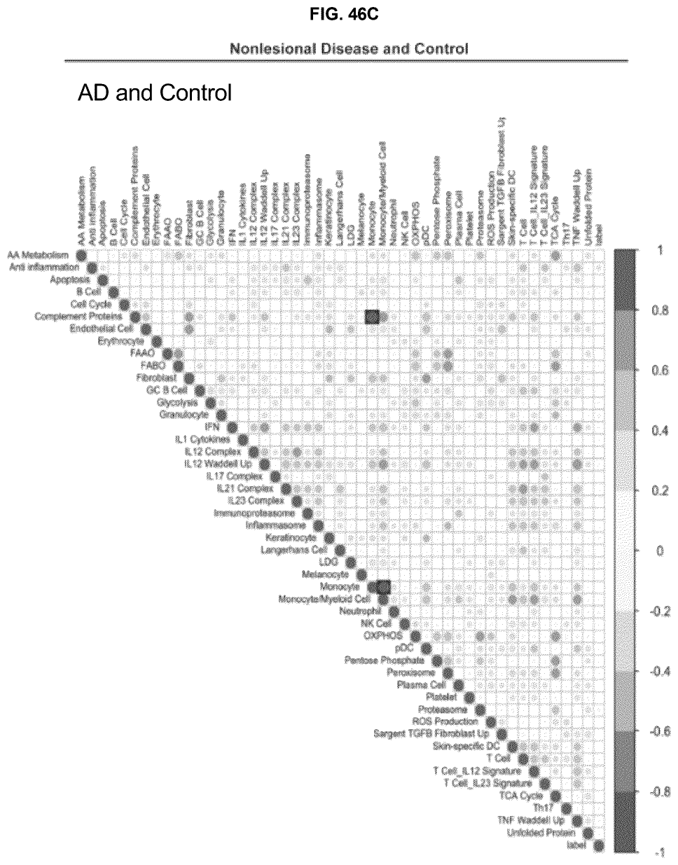

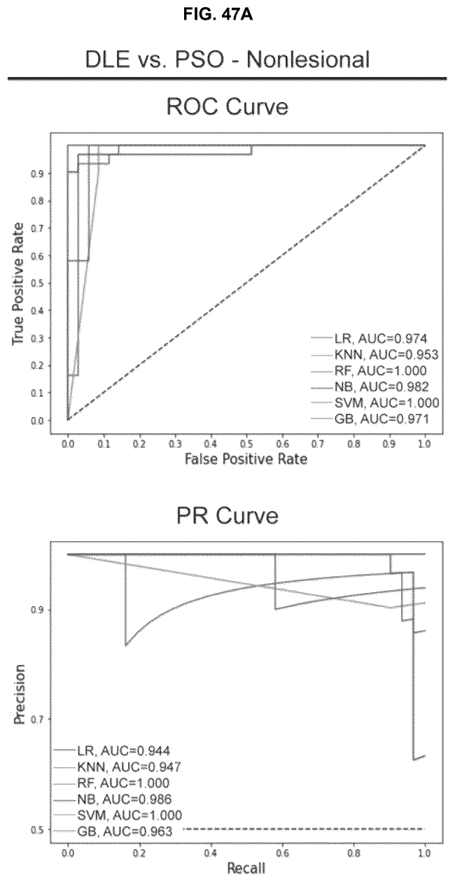

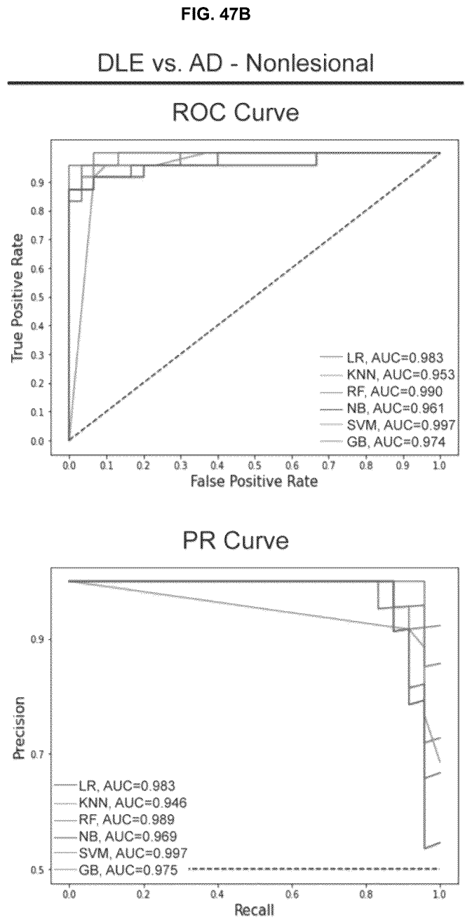

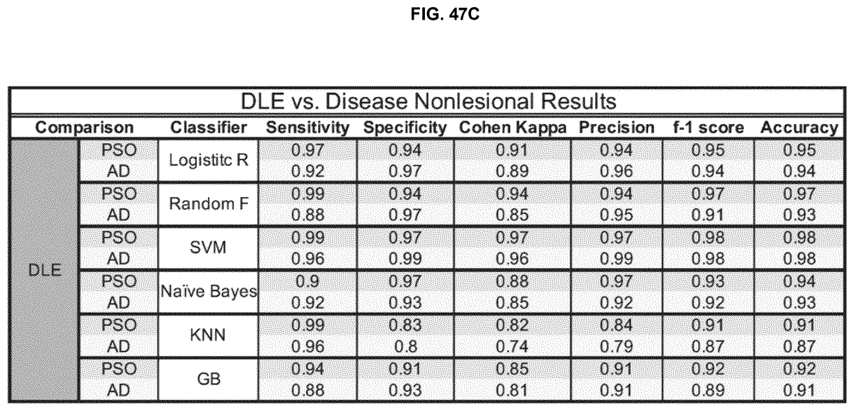

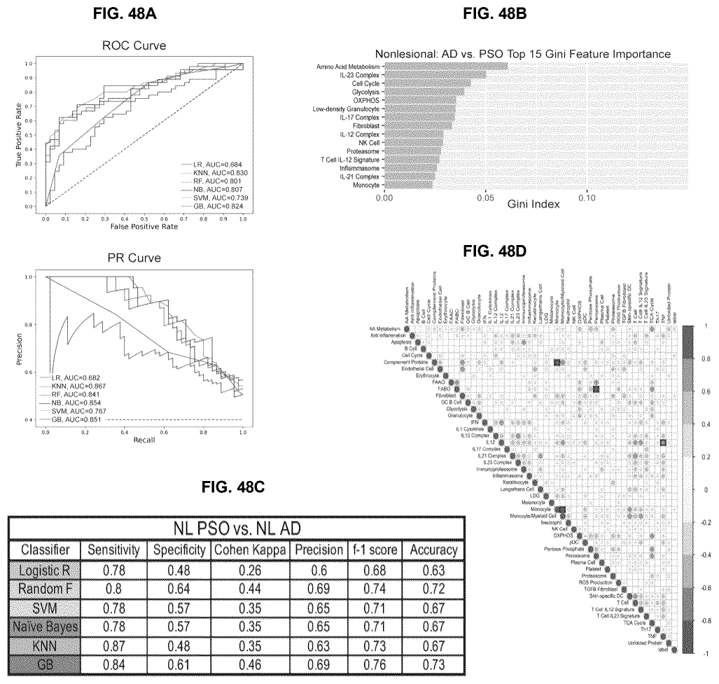

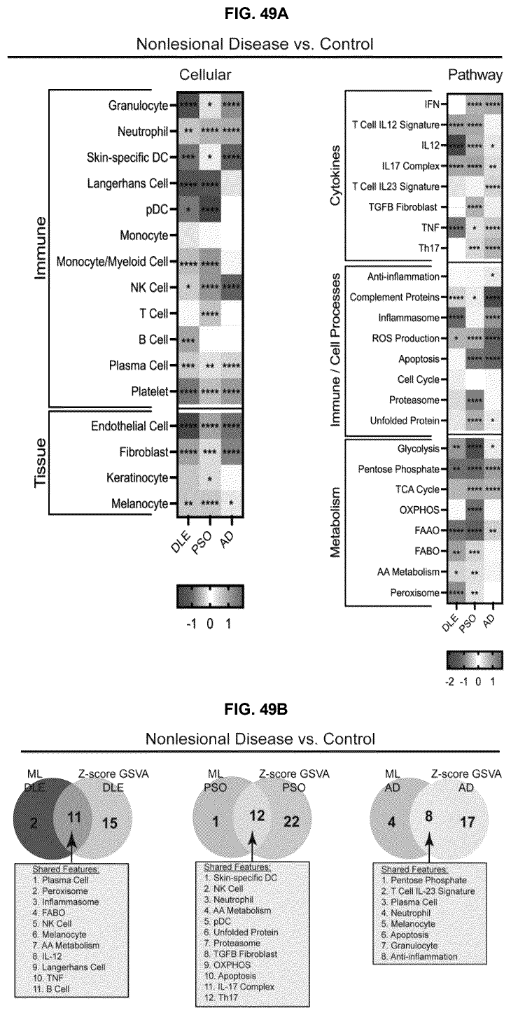

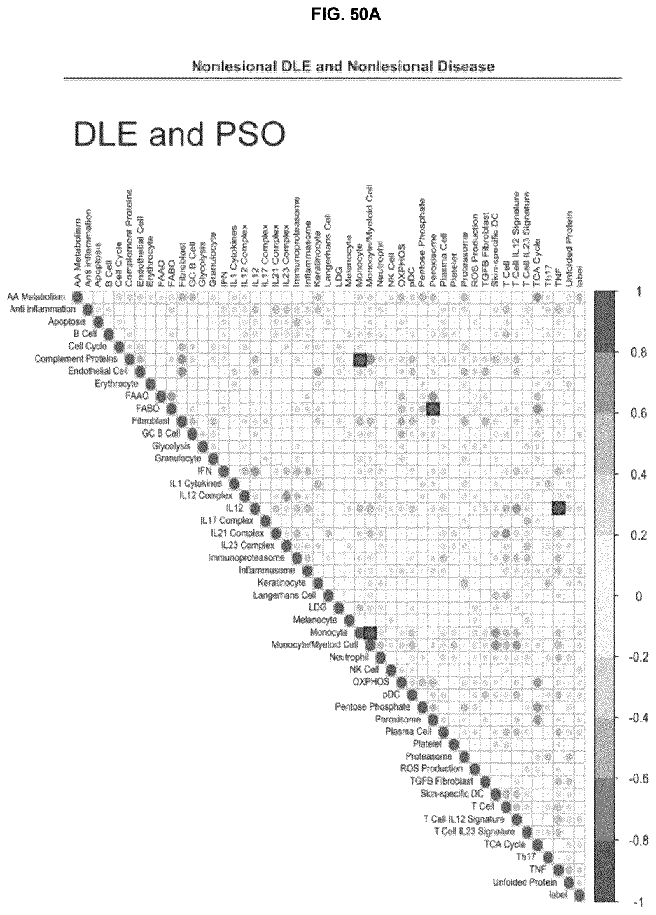

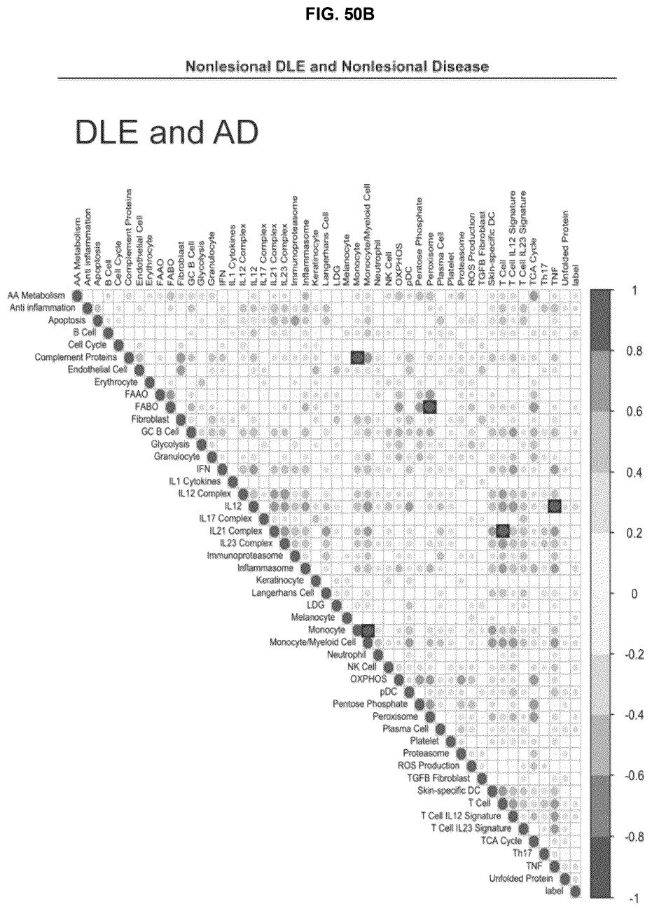

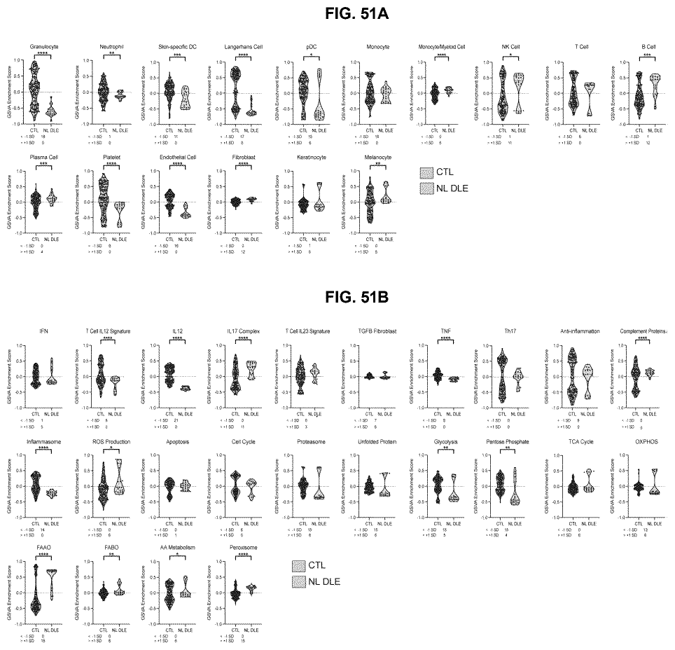

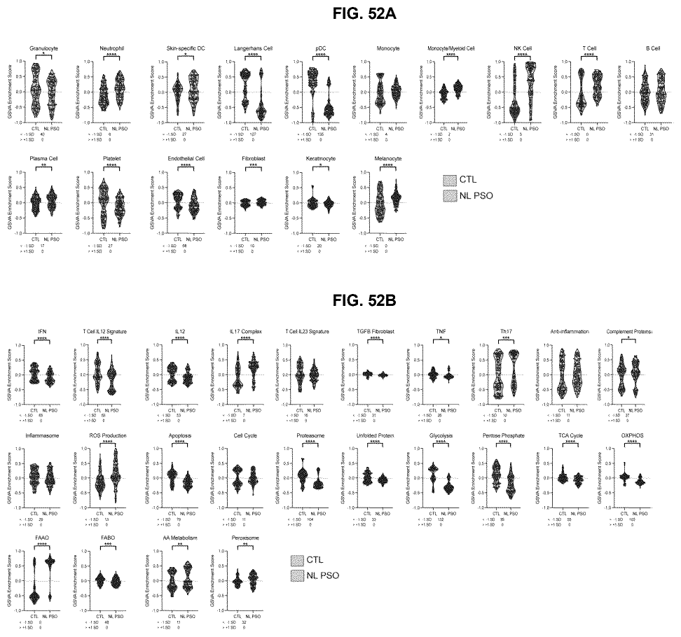

Given that nonlesional skin of each disease was distinct from control skin and appeared to be distinct from other diseases, we next compared our binary classification of nonlesional DLE compared to nonlesional PSO or nonlesional AD using balance strategies described above (FIG. 45A, B). We found that nonlesional DLE and nonlesional PSO are easily separable, with the NK cell, amino acid metabolism and plasma cell signatures being the top features used in classification (FIG. 45C). In the binary classification of nonlesional DLE compared to nonlesional AD, the top features included the inflammasome, NK cell and unfolded protein signatures (FIG. 45D). Comparable classification was observed with the top 15 features only (FIG. 45E, Table 13) and other ML classifiers gave similar results (FIG. 47). Finally, binary classification of nonlesional PSO and nonlesional AD exhibited less effective classifier performance, but classification based on signatures including amino acid metabolism, IL-23 complex and cell cycle was achieved (FIG. 48). These results indicate that transcriptomic profiles of nonlesional skin are sufficiently unique to distinguish different inflammatory skin diseases. Moreover, the differences among nonlesional skin of the three diseases appear to be greater than the differences among lesional skin of the same three diseases.

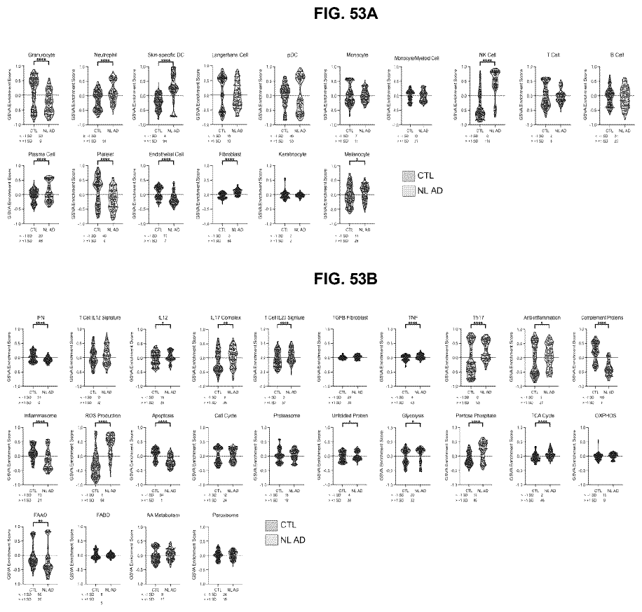

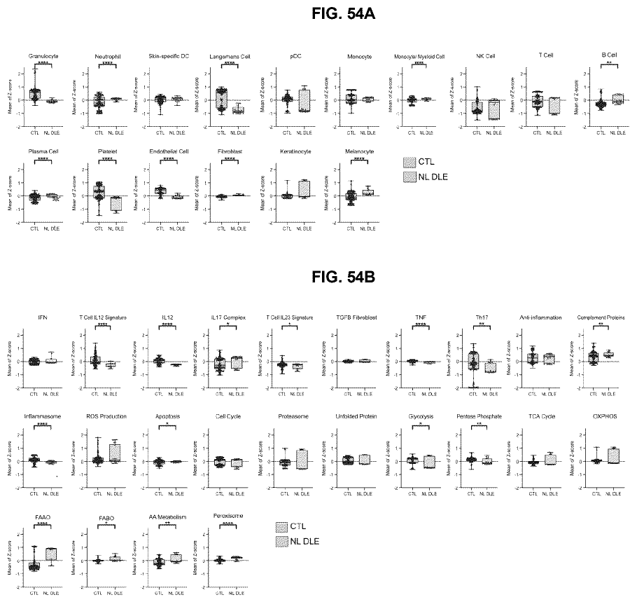

The data demonstrated that ML employed specific gene signatures for the classification of nonlesional samples from patients with inflammatory skin diseases. To probe the differences in nonlesional skin in greater detail, we carried out an additional analysis using GSVA. For this analysis, we pooled the control samples and nonlesional samples from all datasets and employed Z-score normalization to scale data from samples obtained from different datasets. We found that nonlesional DLE samples compared to control samples show upregulation of B cells, melanocytes and complement protein gene signatures (FIG. 49A, FIG. 51). Nonlesional PSO samples showed upregulation of T cell and Th17 gene signatures, whereas nonlesional AD samples compared to control samples showed upregulation of skin-specific DC, IL-12 and anti-inflammation gene signatures as compared to control samples (FIG. 49A, FIG. 52 and FIG. 53).

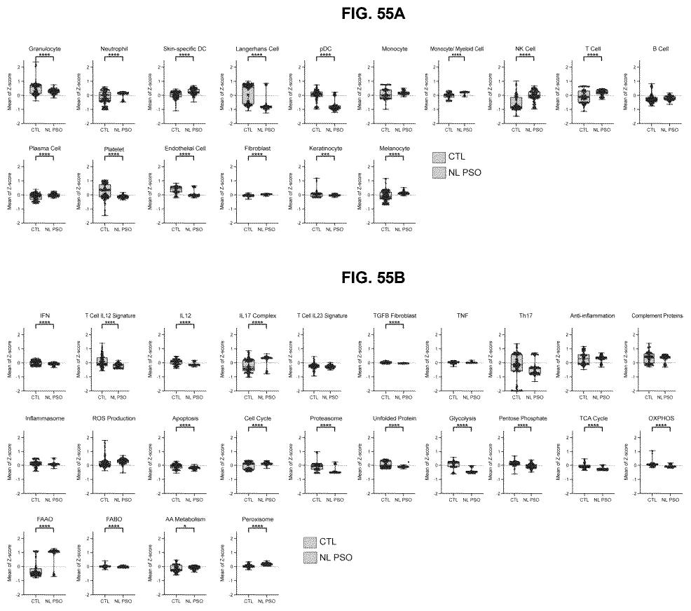

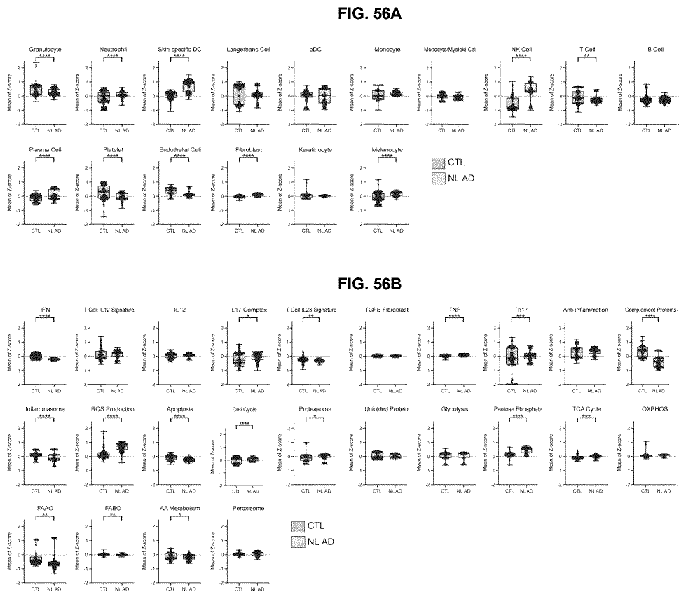

To confirm these GSVA results, we also employed a mean of Z-scores calculation for enrichment of signatures and found similar results (FIGS. 54, 55, 56, Table 6). Notably, comparison of significantly enriched signatures determine by the Z-score GSVA approach and the top most important features for ML classification of nonlesional skin demonstrated considerable overlap (FIG. 49B). Of the 40 features used in both approaches, the majority of discriminatory features were similar. For example, in nonlesional DLE there were 11 shared features between ML and Z-score GSVA methods including the plasma cell, TNF, Langerhans cell and B cell signatures. In nonlesional PSO, there were 12 shared features between the two methods including the skin-specific DC, IL-17 complex and Th17 signatures. In nonlesional AD, there were eight shared features between the two methods including neutrophil, plasma cell and anti-inflammation signatures.

### ML Successfully Classifies Different Subtypes of CLE

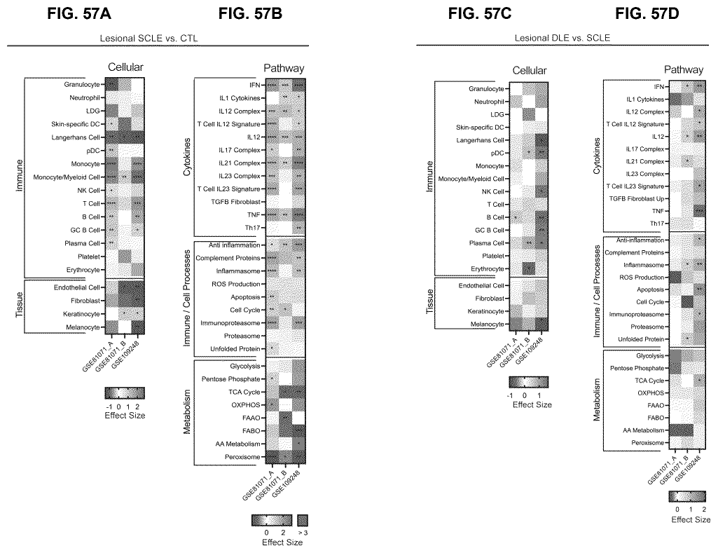

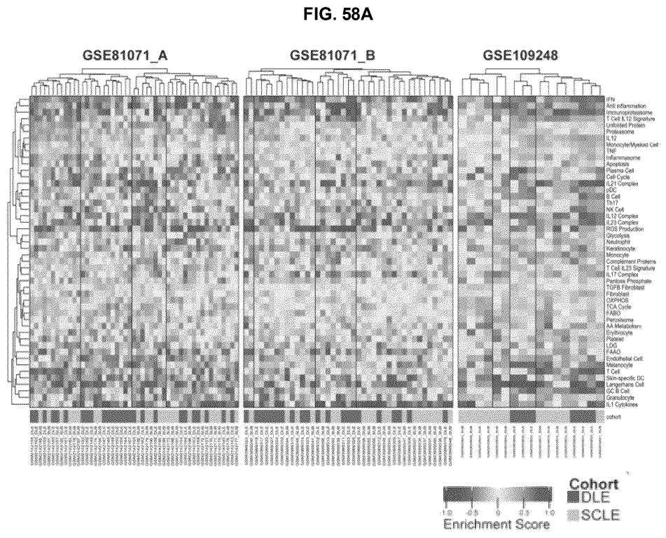

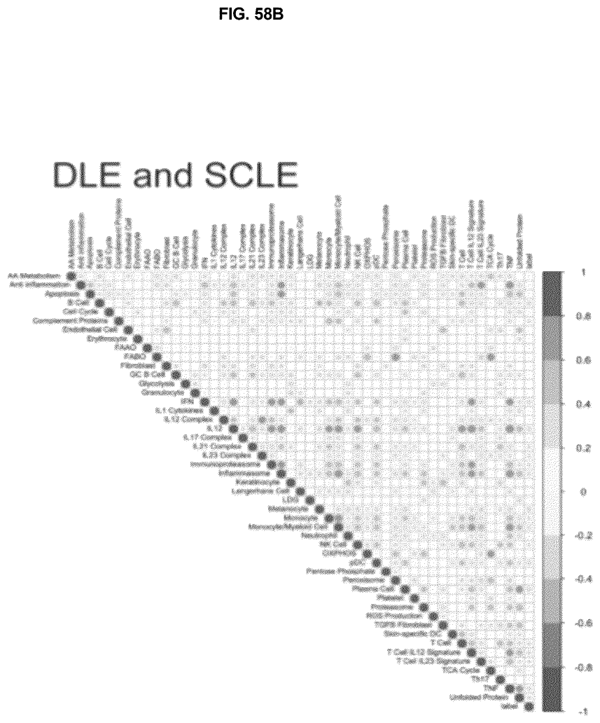

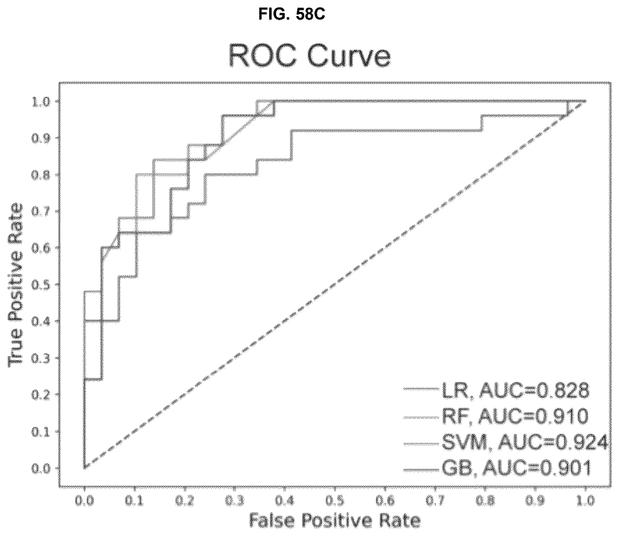

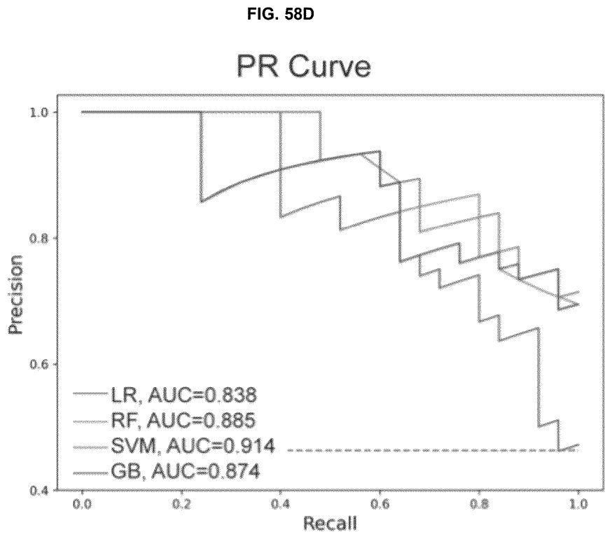

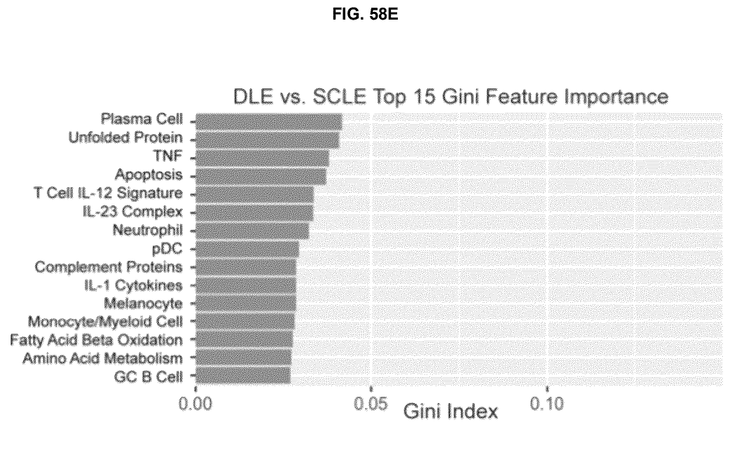

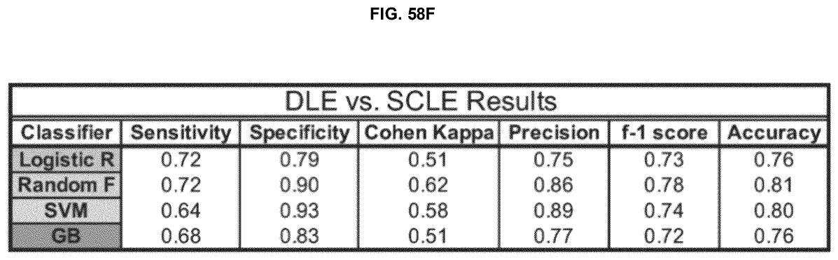

Because the ML pipeline was able to determine specific signatures that separated related diseases, we sought to determine whether subtypes of CLE could be distinguished by analyzing gene expression profiles. We previously observed a robust upregulation of immune cell gene signatures, including the pDC, monocyte, T cell and B cell signatures by GSVA comparison of DLE and control samples (refer to FIG. 29B). The monocyte, T cell and B cell signatures were upregulated and the Langerhans cell signature was downregulated in patients with SCLE as compared to healthy skin in a similar pattern to DLE (FIG. 57A, B). In contrast, comparison of DLE and SCLE revealed minimal signatures with significant differences between the two CLE subtypes (FIG. 57C, D). Indeed, by hierarchical clustering, samples from patients with SCLE did not form distinct clusters but rather were interspersed with DLE suggesting that subtypes of lupus are not molecularly separable (FIG. 58). However, when we applied ML to determine whether DLE could be distinguished from SCLE (FIG. 58A, B), classification with good performance characteristics was achieved (FIG. 58C, D). The top 15 features important in distinguishing DLE from SCLE included plasma cell, unfolded protein and TNF signatures (FIG. 58E). It is notable that the pattern of gene enrichment signatures is similar in DLE and SCLE, but the magnitude of enrichment of gene signatures was greater in DLE. These quantitative differences are sufficient for ML to classify the two subtypes of CLE effectively (FIG. 58F).

### Cytokine-Stimulated Keratinocyte Signatures and T Cell Signatures Differ Among Inflammatory Skin Diseases

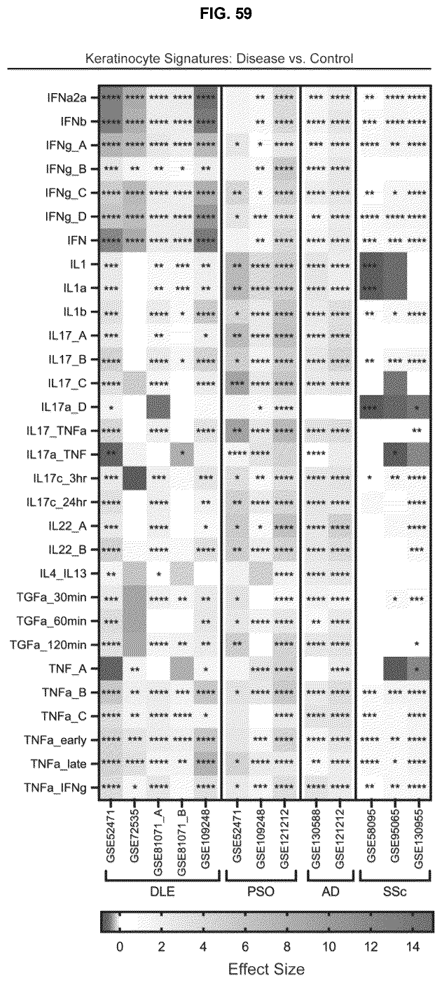

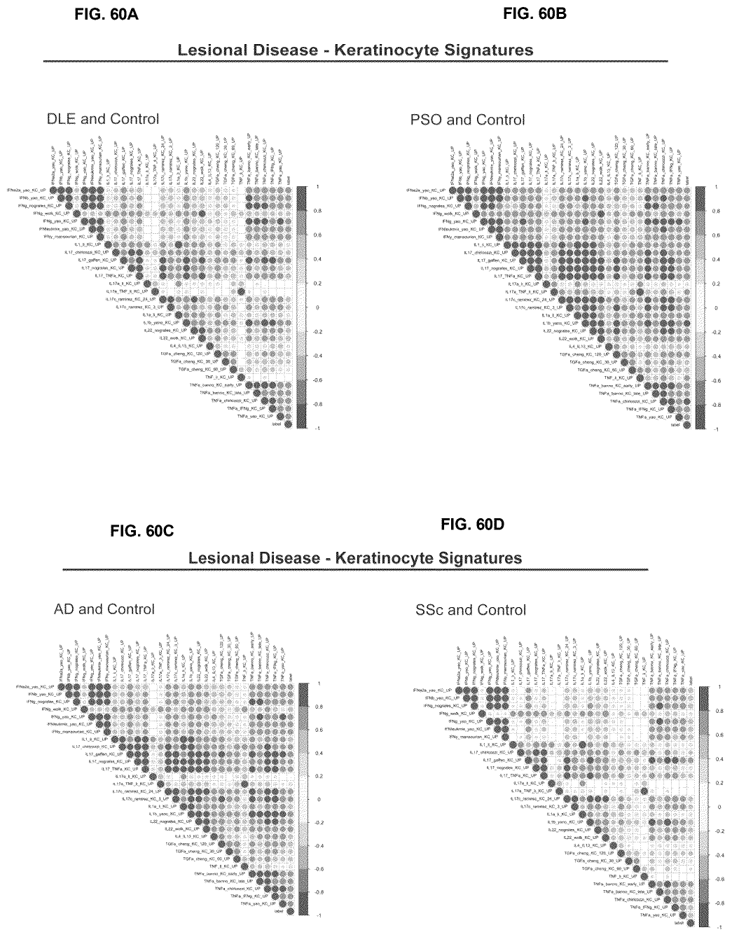

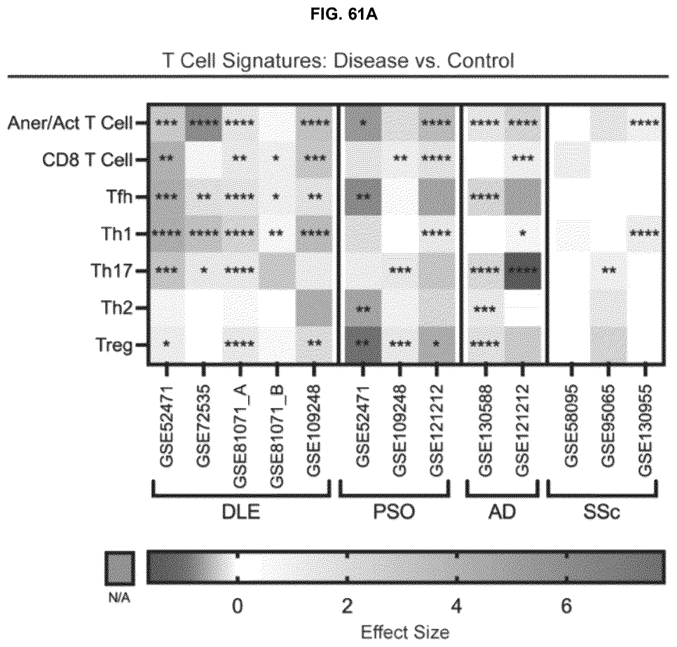

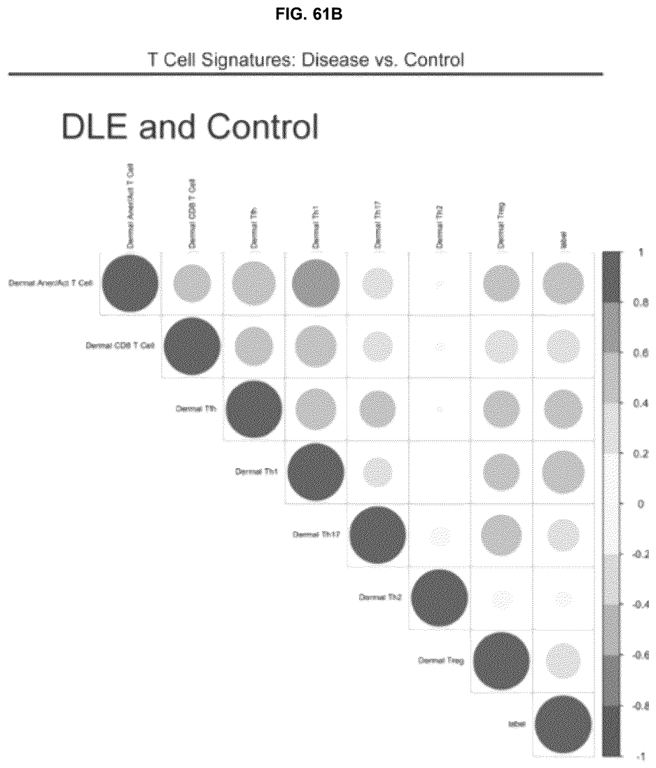

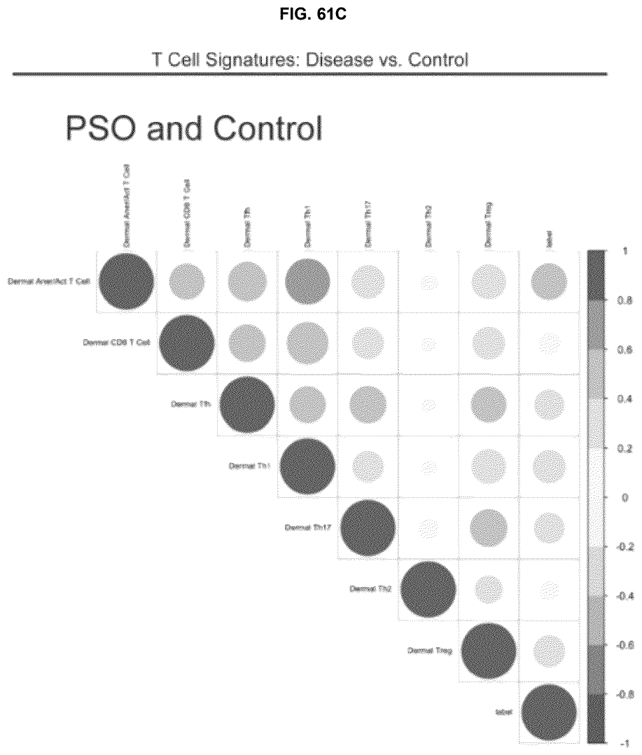

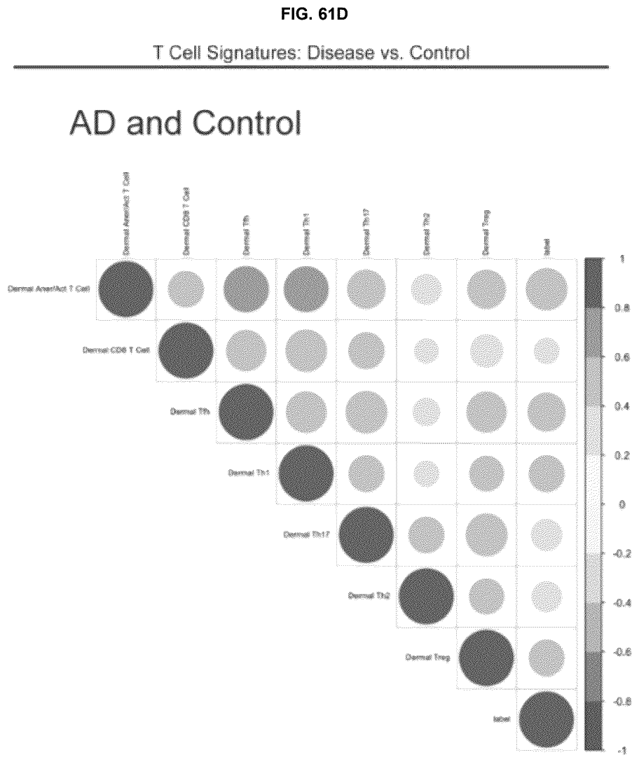

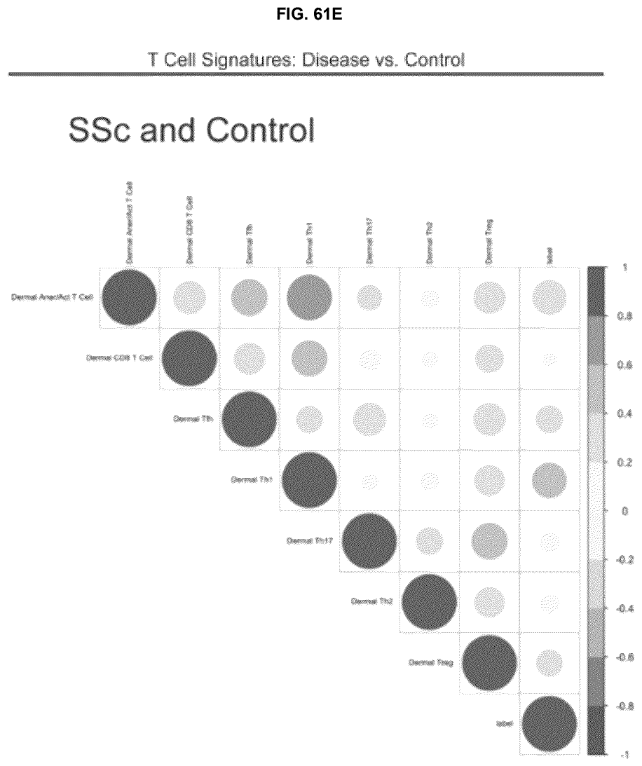

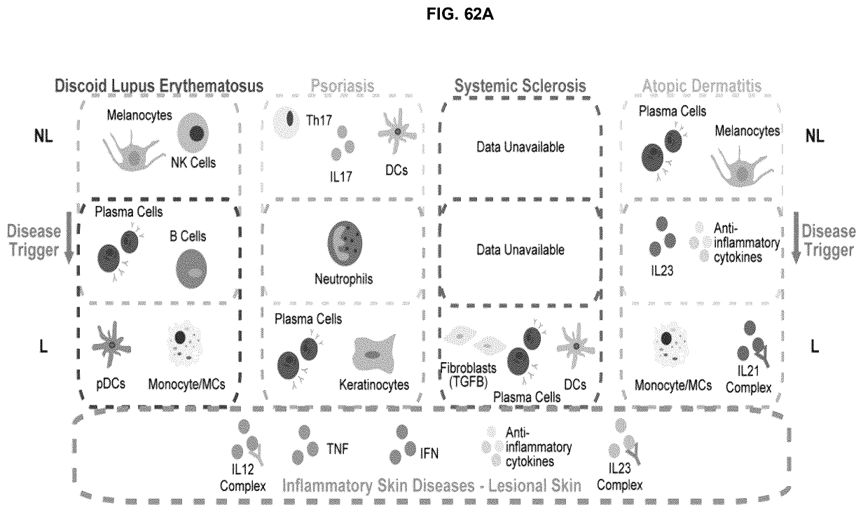

Keratinocyte and T cell signatures are often upregulated in inflammatory skin diseases. Because the ML analyses to this point have focused on gene signatures previously implicated in lupus (17, 21, 22), with less emphasis on those implicated in other inflammatory skin diseases (23, 24), we examined previously published PSO- or AD-specific gene signatures were also differentially enriched among the four lesional inflammatory skin diseases as compared to healthy controls. To accomplish this, we first evaluated gene sets derived from keratinocytes stimulated with various cytokines (Table 4C). We found, in all diseases, that many of the keratinocyte gene signatures were highly enriched (FIG. 59). DLE was highly enriched for the IFN-stimulated keratinocyte gene signatures, whereas PSO was highly enriched in IL-1 and IL-17-stimulated keratinocyte gene signatures. Keratinocyte gene signatures were upregulated equally in AD, including IFN and IL-17-stimulated keratinocyte gene signatures. SSc showed more modest increases to the keratinocyte gene signatures, with IL-1, IL-17 and TNF-stimulated keratinocyte signatures decreased in some datasets. These data indicate a possible role for cytokine-stimulated keratinocytes in the pathogenesis of each disease, with a less prominent effect in SSc. Collinearity analysis revealed that the keratinocyte signatures were highly correlated, and therefore, not appropriate for use in ML (FIG. 60). Secondly, we examined the role of more nuanced T cell populations. For example, Th17 cells and IL-17 are targets of successful therapeutic intervention in PSO(25, 26); additionally, the Th2 T cell subset is implicated in AD pathogenesis (20). GSVA analysis of the T cell gene signatures demonstrated heterogeneity across the PSO, AD and SSc datasets. T follicular helper (Tfh) cell, Th1 cell and Th17 cell signatures were enriched in most DLE datasets; PSO had variable enrichment of Th1 cell, Th2 cell and Th17 signatures, as did AD, while SSc exhibited less robust T cell enrichment (FIG. 61).

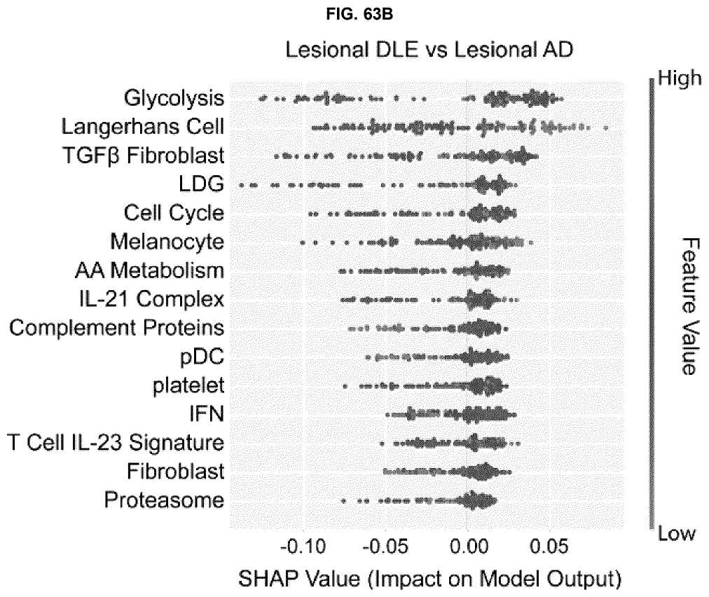

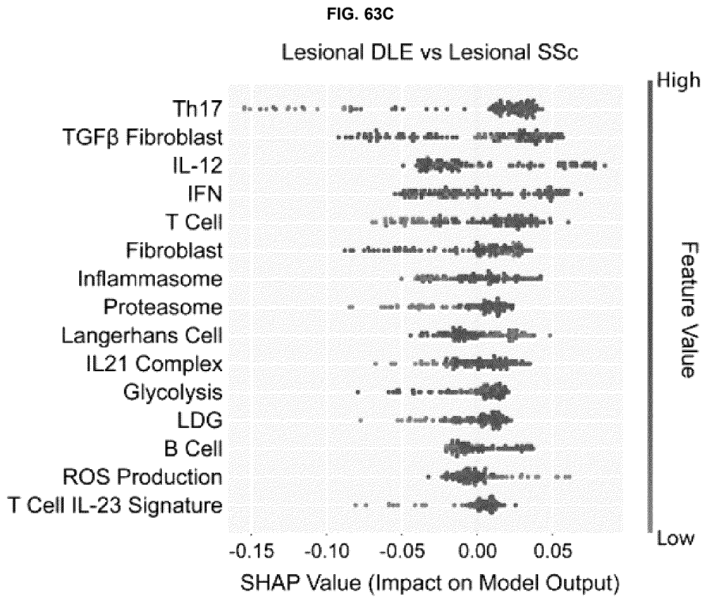

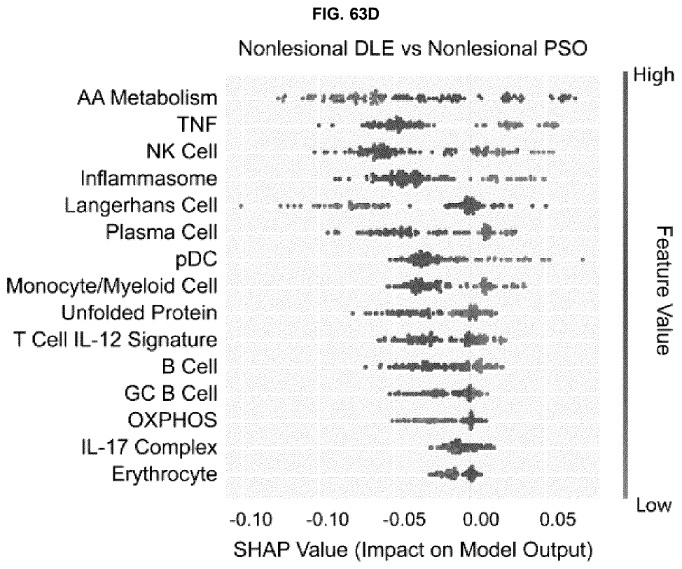

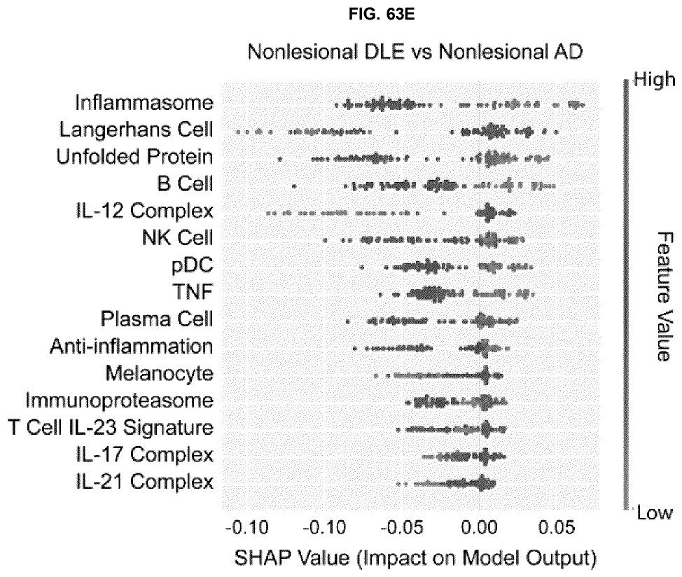

FIG. 63 shows that ML classification of DLE versus PSO, AD, and SSc confirms distinct disease-specific gene signatures. FIGS. 63A, B, and C: Top 15 features important in classifying lesional DLE versus lesional PSO (FIG. 63A), lesional DLE versus lesional AD (FIG. 63B), lesional DLE versus lesional SSc (FIG. 63C). FIGS. 63D, and E: Top 15 features important in classifying nonlesional DLE versus nonlesional PSO (FIG. 63D), and nonlesional DLE versus nonlesional AD (FIG. 63E) using SHAP values. Collinear features were removed. For the classification of DLE vs each disease for both lesional and nonlesional samples, the data set was split into train (70%) and test (30%) sets. Training model weights of the random forest classifier (RF) from each binary classification model (lesional DLE vs lesional AD, lesional DLE vs lesional PSO, lesional DLE vs lesional SSC, nonlesional DLE vs AD, and nonlesional DLE vs PSO) were imported. SHAP was applied on the random forest training model and summary plots (FIGS. 63A to E) of the training data in each disease-to-disease binary classification were generated. The dots in the summary plots refer to the data points/samples. The y-axis of the plot has the features sorted in rank order of their feature importance scores (i.e. high on the top), whereas the x-axis refers to the mean SHAP values of each feature, meaning the magnitude by which the feature impacts the model outcome. The positive SHAP values designate that the feature (the high or low GSVA score) positively contribute to classification of the target variable (DLE=1) and the negative SHAP values designate that the feature negatively contributes feature to the label (in this case, DLE).

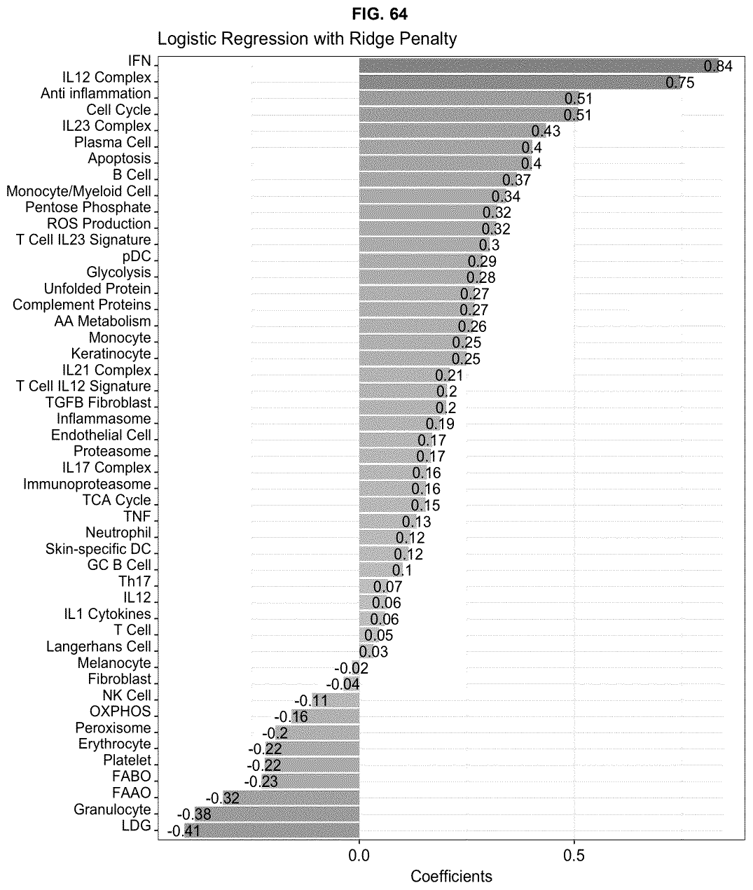

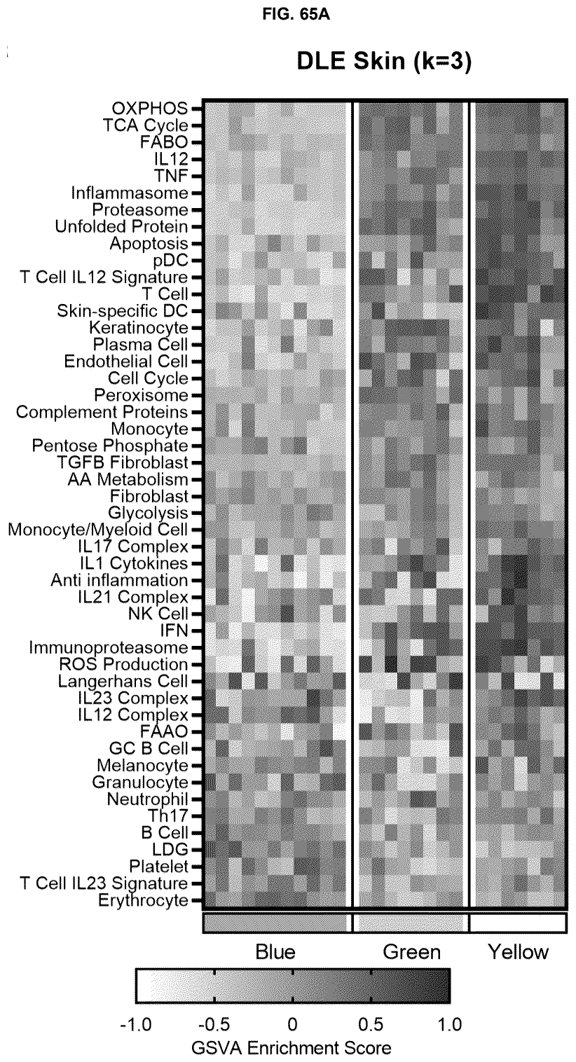

Inflammatory skin disease risk score was calculated to understand activity of cellular and immune pathways in lesional skin diseases. GSVA of 48 gene signatures representing cells (immune and non-hematopoietic) (Tables 4A-1 to 4A-20) and pathways (Tables 4B-1 to 4B-20) was run independently on 16 datasets including samples from DLE, AD, PSO, and SSc. When pooled, the datasets comprised 90 lesional DLE, 183 lesional PSO, 132 lesional AD, 97 lesional SSC and 164 normal skin controls. To derive a model by which the inflammatory skin disease risk score could be generated, the GSVA scores in each sample were binarized, where GSVA scores>0 became 1, and GSVA scores<0 became 0. Logistic regression with ridge penalty was then run by random sampling with replacement 41 samples from each disease (41 lesional DLE, 41 lesional PSO, 41 lesional AD, and 41 lesional SSc), thereby totaling to 164 lesional skin disease samples, and 164 non-disease skin controls on each iteration, with the 48 binarized GSVA scores in each sample serving as features. Coefficients were calculated for each iteration and final coefficients were obtained by taking the average of all iterations. FIG. 64 shows the resulting coefficients for classification of lesional skin from control after the ridge regression model was run for 500 iterations. To calculate inflammatory skin disease risk score in each sample, the coefficient for each category from the logistic regression model is multiplied by the binarized GSVA score for that category and all categories are summed to generate a final score. Following are the resulting coefficients shown in FIG. 64—IFN: 0.84; IL 12 complex: 0.75; Anti inflammation: 0.51; Cell cycle: 0.51; IL 23 complex: 0.43; Plasma Cell: 0.4; Apoptosis: 0.4; B Cell: 0.37; Monocyte/myeloid Cell: 0.34; Pentose Phosphate: 0.32; ROS production: 0.32; T Cell IL 23 Signature: 0.3; pDC: 0.29; Glycolysis: 0.28; Unfolded Protein: 0.27; Complement Protein: 0.27; AA Metabolism: 0.26; Monocyte: 0.25; Keratinocyte: 0.25; IL 21 Complex: 0.21; T Cell IL 12 signature: 0.2; TGFB Fibroblast: 0.2; Inflammasome: 0.19; Endothelial Cell: 0.17; Proteasome: 0.17; IL 17 Complex: 0.16; Imunoproteasome: 0.16; TCA cycle: 0.15; TNF: 0.13; Neutrophil: 0.12; Skin-specific DC: 0.12; GC B cell: 0.1; Th17: 0.07; IL 12: 0.06; IL1 cytokines: 0.06; T Cell: 0.05; Langerhans Cell: 0.03; Melanocyte: −0.02; Fibroblast: −0.04; NK Cell: −0.11; OXPHOS: −0.16; Peroxisome: −0.2; Erythrocyte: −0.22; Platelet: −0.22; FABO: −0.23; FAAO: −0.32; Granulocyte: −0.38; and LDG: −0.41.

Analysis of the Transcriptomic Profiles of Rheumatic Skin Diseases Reveals Disease-specific Endotypes. Patients with rheumatic skin diseases such as cutaneous lupus erythematosus (CLE) and systemic sclerosis (SSc) can be classified from individuals with healthy skin using the enrichment of specific molecular signatures However, as patient heterogeneity is a well-known feature of these diseases, it is important to ascertain whether there are distinct patient molecular phenotypes (endotypes). Moreover, identifying specific disease endotypes from analysis of peripheral blood might increase the capacity to recognize patient endotypes.

Gene expression data derived from publicly available lesional skin biopsies of CLE, specifically discoid lupus erythematosus (DLE), the most severe CLE subtype, and SSc patients was analyzed using gene set variation analysis (GSVA) of informative gene modules. Paired blood samples were also analyzed when available. K-means clustering was then applied to the GSVA enrichment scores to identify molecular endotypes in both skin diseases.

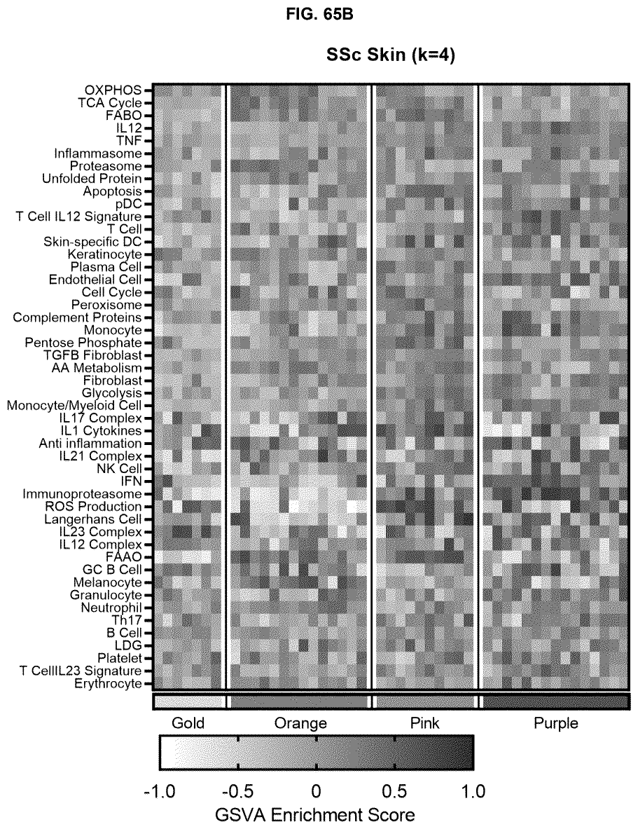

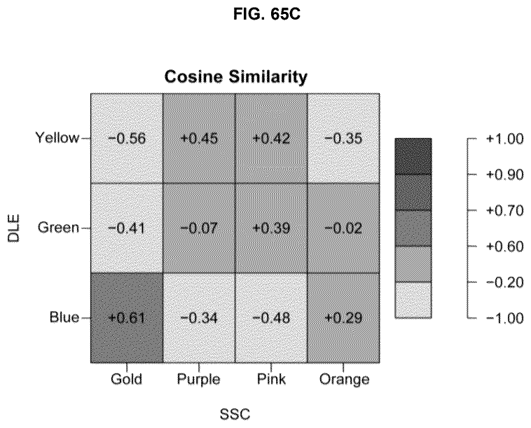

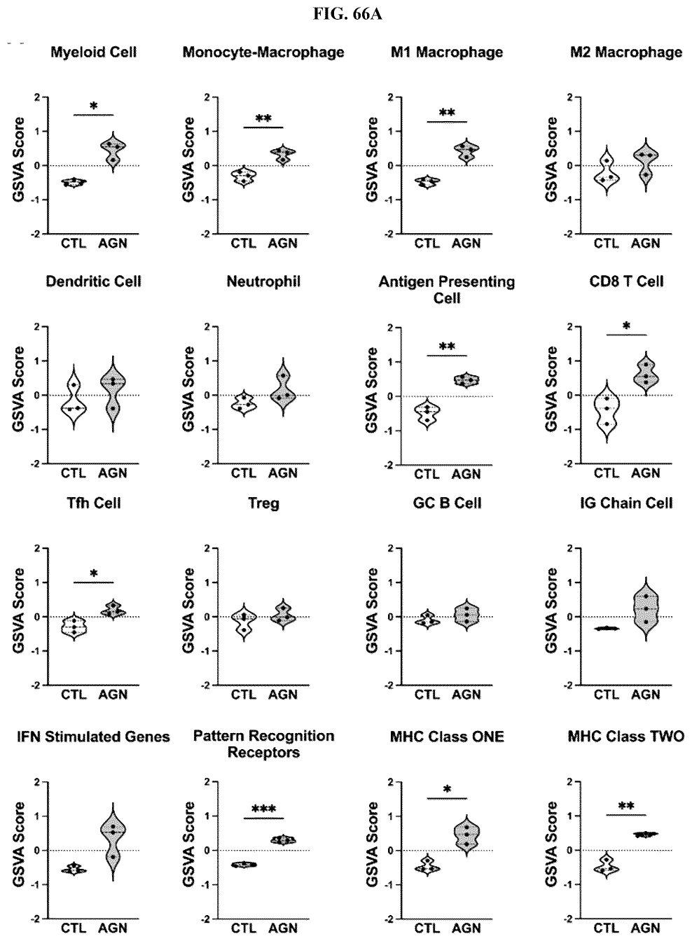

In DLE, k-means clustering revealed three subsets with distinct features (FIG. 65A). The least abnormal DLE cluster (blue, Group 1) was characterized by minimal changes, including higher B cell GSVA scores, whereas the other two clusters (Green, Group 2 and Yellow, Group 3) had markedly increased inflammatory cell and pathway gene expression, with most modules being enriched by a greater magnitude in yellow (Yellow, Group 3). In SSc, four endotypes emerged from k-means clustering (FIG. 65B). The first cluster (Gold, Group 1) was the least abnormal, with unchanged cell and metabolic signatures. The second cluster (Orange, Group 2) was characterized by increased melanocyte transcripts and altered metabolic signature expression. The third cluster (Pink, Group 3) exhibited increased myeloid, NK, T, and plasma cell signatures as well as increased glycolysis, pentose phosphate, and apoptosis signatures. The most severe SSc endotype (Purple, Group 4) was comprised of increased myeloid cell, T cell, and fibroblast signatures, and altered metabolism expression. Both the pink and purple clusters were enriched in cytokine signaling modules such as IL12 and TNF. Comparison of the three DLE endotypes with the four SSc endotypes revealed some overlap between the least severe clusters (blue and gold) of each disease (FIG. 65C).

Both DLE and SSc skin exhibit distinct subsets based upon their molecular profile (endotype). In addition, the most severe SSc skin subset can be identified from blood gene expression. Identifying specific molecular endotypes of patients with inflammatory skin disease may facilitate matching individual patients with effective therapies.

### Discussion

Here, we employed a comprehensive analysis of gene expression profiles to characterize the molecular features of four inflammatory skin diseases. Although considerable inter- and intra-dataset heterogeneity was observed, we documented molecular gene signatures that define both lesional and nonlesional skin of the various conditions. Notable among the findings were the shared and unique features of lesional skin among the four diseases and the unique features of nonlesional uninvolved skin. Altogether, this analysis demonstrates the informative power of transcriptomics to determine pathological characteristics of specific stages of each disease.

Our analyses involved multiple bioinformatic and statistical approaches that allowed us to understand the molecular pathways underlying the pre-clinical and clinical stages of inflammatory skin diseases. First and foremost, we assessed numerous datasets for each disease so that we could capture the transcriptional landscape of each condition and overcome the heterogeneity among patients and datasets. Second, each dataset was independently evaluated by GSVA using informative gene signatures we previously employed in the analysis of lupus (17, 21, 22, 27), gene signatures derived from interrogation of other inflammatory skin diseases (23, 24), as well as additional signatures we generated because of their relevance to skin pathogenesis. This analysis allowed us to observe unique patterns in the enrichment of inflammatory pathway signatures among and between the diseases and document that the diseases were molecularly separable. We employed ML models, including CART and random forest, to determine that effective classification between disease and control or between diseases was achievable and to identify the most important features labeling the conditions. The ML models not only permitted the effective classification of samples, but also allowed for dimensionality reduction, scaling the original 48 input gene signatures down to 15 features most important in each classification.

Despite previously noted heterogeneity (28, 29), our analysis revealed that the molecular landscape of DLE was more homogeneous across datasets comprising patients from different centers was sufficiently similar to permit accurate classification. Similarly, datasets including patients with SSc demonstrated consistent gene expression patterns. In contrast, we and others found datasets comprising patients with PSO and AD to be more molecularly heterogeneous (30). Nevertheless, we identified definitional transcriptional elements for each of the various conditions that included both shared and specific molecular perturbations. Comparison of the lesional DLE, PSO, AD and SSc transcriptomes using GSVA demonstrated that these four inflammatory skin diseases have numerous inflammatory pathways in common. IFN, TNF, IL-12complex, IL-23 complex, T cell IL-23, anti-inflammation and unfolded protein gene signatures were commonly upregulated among lesional biopsies from the four inflammatory diseases. Indeed, CART analysis, which was used as a first path algorithm to detect important discriminators within the data, demonstrated the IFN and IL-12 complex gene signatures were the two most important features in distinguishing lesional DLE, PSO, AD and SSc from their respective control samples. Moreover, ML algorithms documented that of the 15 features necessary for accurate classification of each disease from control, seven features are common among all four diseases, including the IGS, IL-12 complex, IL-23 complex, TNF, plasma cell, T cell IL-23 and anti-inflammation gene signatures. Altogether, there were six shared and upregulated features between the GSVA and ML methods, suggesting that despite different genetic predispositions and disease manifestations, lesional DLE, PSO, AD and SSc have a common inflammatory microenvironment that differentiates them from control skin. This was further supported by the overlapping enrichment of numerous signatures among at least two of the four diseases. For example, GSVA demonstrated the neutrophil signature was upregulated in the majority of PSO and SSc patients, whereas the pDC, monocyte, monocyte/myeloid cell and B cell signatures were increased in the majority of DLE and SSc patients; the T cell, IL-21 complex, inflammasome and cell cycle signatures were increased in all diseases, except SSc.

Despite the similarities among the diseases however, we detected unique characteristics of each inflammatory skin disease. Indeed, we observed clear molecular distinctions between lesional samples from patients with DLE, PSO, AD, and SSc. The GSVA analysis revealed that the NK cell signature was only upregulated in lesional DLE compared to control samples, whereas the IL-1 cytokine signature was uniquely upregulated in lesional PSO compared to controls. Notably, however neither signature proved to be of particular importance in ML classification of either disease from controls. Similarly, the proteasome and Langerhans cell signatures were uniquely enriched in AD compared to control samples, and the endothelial cell and fibroblast signatures were uniquely enriched in lesional SSc compared to controls, these signatures were not of particular importance in ML classification of either disease. Despite this complexity, ML was able to delineate the most important features for classification of each condition from normal. For example, although increased in some patients from all diseases, the monocyte, T cell and B cell signatures were more important in the classification of DLE. Moreover, the keratinocyte and neutrophil signatures were most important in classifying PSO, and not the other diseases, a finding that is consistent with the role of keratinocyte proliferation and neutrophil infiltration in PSO (18, 31-33). In addition, the IL-21 complex signature was upregulated in all diseases except SSc, but was unique to ML classification of AD, consistent with the role of IL-21 in allergic skin diseases (34, 35). Finally, the TGFB fibroblast signature, was important in classification of SSc, which aligns with the central role of fibrosis in this disease (23, 36). Furthermore, ML demonstrated that the pDC, fibroblast, and glycolysis signatures are important in classifying lesional DLE from the other lesional diseases, illustrating that ML can identify molecular changes as effective classification features among samples. These findings strongly imply there are unique molecular features in lesional biopsies of inflammatory skin disease, along with a panoply of shared features.

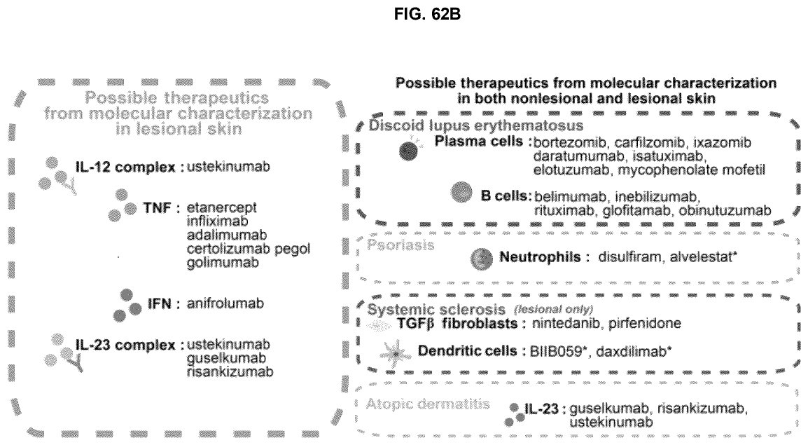

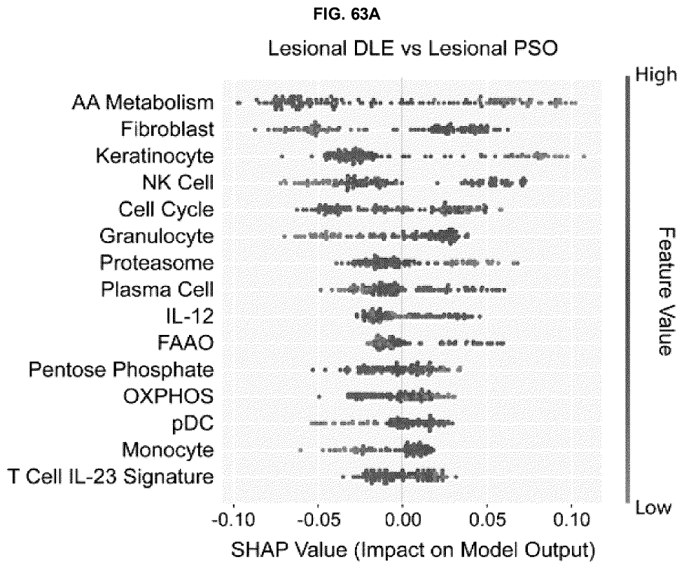

Although there are numerous reports of gene expression abnormalities in lesional skin, less is known about the architecture of clinically uninvolved skin as compared to healthy skin. Examination of nonlesional skin in DLE, PSO, and AD provided new insights into the molecular processes operating in uninvolved skin and suggested a unique pre-clinical set of abnormalities in each condition. Application of both ML and Z-score based approaches as orthogonal analytic tools to assess the differences between nonlesional and normal skin revealed unique patterns of abnormalities in each inflammatory skin condition. Notably, only the apoptosis signature was one of the top 15 features employed by ML to classify nonlesional DLE, PSO, and AD versus pooled controls (FIG. 62A). FIG. 62B shows summary of possible therapies of lesional skin diseases analyzed (left) and possible therapies for both lesional and nonlesional regions of each disease (right) based on molecular characterization. This suggests that dysregulated apoptosis may be a key feature in the initiation of each of these three diseases; indeed, apoptosis is cited in the pathogenesis of skin diseases including CLE, PSO and AD and enhanced apoptosis is a well-recognized systemic feature of SLE (37-41). In general, unique molecular features characterize each condition, such as the IL-21 pathway in CLE and IL-17 in PSO. Genetic polymorphisms may contribute to the abnormalities noted in nonlesional skin, as for example, susceptibility to lupus is in part associated with polymorphisms in IL-21 (42) and the IL-21 receptor (43) and polymorphisms in IL-17 are correlated with PSO treatment response (44).

Of note, unlike lesional disease, we did not observe a prominent role for the IGS in nonlesional skin from DLE, PSO, or AD. This contrasts with some previous studies suggesting that nonlesional skin from patients with SLE or DLE is influenced by type 1 IFN (45-47). However, this contention was based largely on single-cell RNA-seq analysis of nonlesional keratinocytes and their expression of the IGS (45, 46), whereas our studies have evaluated expression of the IGS by deconvolution of bulk tissue gene expression. Our data revealed increased IGS in a few DLE samples, which may align with the increase of IFN action in only select cell clusters from single-cell RNA-seq analysis, but not in the majority of samples. Altogether, this suggests that IFN is not a dominant factor of nonlesional disease in either CLE or PSO and may instead reflect the concurrent exposure to UV light or presence of specific autoantibodies, both of which are associated with upregulation of the IGS (48-50).

Taken together, the data suggest a model in which patients with inflammatory skin disease manifest a specific set of pre-clinical molecular abnormalities that could predispose a patient to the development of typical clinical features, perhaps after encountering an environmental trigger (such as UV light, bacterial products or allergens). Upon development of cutaneous inflammation, common molecular features are upregulated, although the lesional disease maintains a unique gene expression profile (FIG. 62). This model is consistent with reports that nonlesional skin of patients with inflammatory skin disease is a pre-inflammatory or “primed” state, and that some of the same molecular processes may contribute to maintaining both the pre-inflammatory and inflammatory components of the skin conditions (30). Herein, we see both an overlap of gene signatures upregulated in lesional skin between skin conditions but also some overlap between nonlesional and lesional skin within each inflammatory skin disease.

Previous reports were not able to separate molecular features of DLE from those of SCLE despite the dramatic differences in clinical phenotype (51). Both DLE and SCLE are characterized by interface dermatitis, but the differences in clinical manifestations suggest different molecular underpinnings. By GSVA, gene expression profiles of these two entities were similar to each other. In fact, GSVA analysis showed the same gene signatures were significantly enriched in each subtype compared to control. However, the effect size was greater in significantly enriched modules in DLE compared to SCLE. The quantitative differences were sufficient for ML to classify DLE from SCLE by using predominately the plasma cell, neutrophil, pDC, melanocyte and GC B cell features as well as the TNF, IL-12 and IL-1 cytokine inflammatory features to classify CLE subtypes.

The results of this analysis lend insight into future treatment strategies for CLE, PSO, AD, and SSc based on the observed common and distinct molecular characteristics. For example, IL-17 is a well-known target for PSO treatment (52) and has been explored in therapy for lupus (53) and AD (54); however, we did not observe consistent upregulation of the IL-17 complex signature among the lesional manifestations of DLE, AD, and SSc, suggesting IL-17 neutralizing therapy may be best suited for lesional PSO alone. However, we observed upregulation of IL-17 complex and Th17 gene signatures in nonlesional PSO and AD samples, as well as upregulation of the IL-17 complex signature in DLE as compared to control samples, suggesting that IL-17 targeting might be appropriate to prevent the emergence of typical skin lesions in all three diseases as well as to treat established plaques in lesional PSO. Of note, two of five lesional DLE datasets demonstrated significant upregulated of the IL-17 complex and Th17 signature suggesting that a subset of DLE patients might be responsive to IL-17 neutralization, and a study investigating the role of secukinumab, a monoclonal antibody to IL-17a, in DLE is ongoing (55). In addition, the consistent upregulation of the TNF signature in each lesional inflammatory skin disease supports the possibility that TNF neutralizing agents may ameliorate inflammation in all four conditions. To date, TNF neutralizing agents are effective in treating PSO (26) (etanercept (56), infliximab (57), adalimumab (58), and certolizumab (59)), while others report their possible efficacy in SLE (60) and AD (61, 62). Notably, a recent phase II trial found that intradermal injection of a TNF neutralizing agent, etanercept, as opposed to traditional systemic injection, induced remission in DLE (63, 64), supporting the conclusion that local presence of TNF in the skin lesion is pathogenic in DLE. The IL-12 signature was important in classifying all four lesional skin diseases, suggesting potential efficacy for the IL-12/23 inhibitor, ustekinumab, which is approved for treating PSO(65). Recent phase III trials in lupus were unsuccessful (66, 67), but improvement of skin and mucocutaneous lesions was noted in phase II trials (68). Finally, consistent enrichment of the IGS was noted in lesional skin of all four diseases, suggesting the potential for efficacy of interferon inhibitors such as anifrolumab. Indeed, anifrolumab treatment, which was recently approved for SLE, caused a significant reduction in skin involvement in CLE compared to patients receiving placebo (7).

Some of the individual datasets had small sample numbers; therefore, it was necessary to pool lesional samples from each disease, nonlesional samples from each disease and controls to achieve sufficient sample numbers for ML. In addition, some datasets had few or no controls, and thereby, nonlesional skin could not be compared to control samples by GSVA without the pooling of samples and employing normalization steps. Despite this, we found a number of changes in nonlesional DLE similar to those previously reported by other techniques, for example the decrease in Langerhans cells via immunohistochemistry (69). Moreover, because many of the widely used keratinocyte gene signatures were highly correlated with each other, ML analysis on cutaneous gene signatures previously reported in PSO and AD was not possible (30, 70, 71). Despite these caveats and the intra- and inter-dataset heterogeneity, we identified gene signatures both similar and distinct in lesional and nonlesional inflammatory skin diseases.

In summary, this transcriptomic analysis is one of the first comprehensive studies to evaluate four inflammatory skin diseases concurrently and introduce comparative analyses of both lesional and nonlesional samples with control samples. We elucidated similarities and differences among both lesional and nonlesional DLE, PSO, AD, and SSC. Overall, our combined GSVA/ML analysis demonstrated that although there are seven shared features for classifying lesional DLE, PSO, AD, and SSc from pooled controls; nonlesional skin samples among diseases are molecularly more distinct from one another than lesional samples. This reveals that nonlesional skin samples are extremely informative about the underlying disease process and could be used in a subset of patients for future clinical trials (72). Indeed, nonlesional skin may be more useful in identifying the driving features underlying pathogenesis, since during chronic lesional disease the inflammatory milieu among diseases becomes more similar. In addition, although enrichment analysis of all cell types and pathways is important in the overall definition of disease pathology and necessary to understand for treatment, specific features may be more important in molecular diagnostics for identifying one disease from another.

### Materials and Methods:

Experimental Design: 15 publicly available gene expression datasets (accessed from the Gene Expression Omnibus (GEO)) were analyzed (Table 3), including: 11 Affymetrix/Illumina microarray datasets (GSE52471 (10), GSE72535 (11), GSE81071 (14, 15, 68, 69), GSE109248 (13), GSE100093 (16), GSE120809 (12), GSE117239 (75), GSE117468 (52), GSE130588 (76), GSE58095 (77), GSE95065 and 4 RNA-seq datasets (GSE121212 (30), GSE137430 (54), GSE157194 (71), GSE130955 (78)). GSE81071 was split into two parts based on the submission date on GEO (GSE81071 from 2017 referred to in the text as GSE81071_A and GSE81071 from 2019 referred to in the text as GSE81071_B). All datasets comprise gene expression derived from skin biopsies of lesional or nonlesional skin from patients with an inflammatory skin disease, including PSO, AD, SSc, and CLE subtypes including DLE, SCLE, and ACLE or skin biopsies derived from healthy control subjects. For GSE117239 (75), GSE117468 (52), GSE130588 (76), GSE137430 (54), and GSE157194 (71), only lesional and nonlesional samples at baseline without drug treatment were included in the analysis.

Statistical Analysis: Statistical differences between cohorts were evaluated using unpaired t-test with Welch's correction for GSVA enrichment scores of lesional and nonlesional samples, mean Z-scores of nonlesional samples versus control, and paired t-test with Welch's correction for lesional versus nonlesional comparison were carried out in GraphPad PRISM. Calculation of mean and standard deviation (SD) for each GSVA score in each tissue was performed in Microsoft Excel. The number of samples for each dataset detailed in Table 5B. Further statistical details can be found below.

### Raw Data Processing:

Microarray data: Microarray data was normalized using either GeneChip Robust Multiarray Average (GCRMA), Robust Multiarray Average (RMA), or normexp background correction (NEQC) based on the microarray platform. Outliers and batch effects were identified using principal component analysis (PCA) plots. For the dataset with known batch effects, GSE81071, raw gene expression values were normalized using 11 housekeeping genes, which were shown to not vary significantly across datasets (79). These 11 housekeeping genes were: chromosome1 Open Reading Frame 43 (C1orf43), Charged multivesicular body protein 2A (CHMP2A), ER membrane protein complex subunit 7 (EMC7), glucose-6-phosphate isomerase (GPI), proteosome subunit beta type 2 (PSMB2), proteosome subunit beta type 4 (PSMB4), member RAS oncogene family (RAB7A), receptor accessory protein 5 (REEPS), small nuclear ribonucleoprotein D3 (SNRPD3), valosin containing protein (VCP), and vacuolar protein sorting 29 homolog (VPS29).

RNAseq data: SRA toolkit (NCBI Sequence Read Archive, Version 2.10) was used to fetch .sra files from GEO and convert them to .fastq files. Quality of the FASTQ files was checked using FASTQC software (Babraham Institute Bioinformatics, Version 0.11.9). Adapters were removed using Trimmomatic software (80) (Version 0.4) and appropriate head crop parameters. Trimmed reads were aligned to the human reference genome (hg38) using STAR aligner (81) (Version 2.7). STAR output .sam files were converted to .bam files using sambamba (82) (Version 0.8). Read summarization was provided using the featureCounts (83) function of the Subread (84) (Version 2.0) package. Count normalization and regularized log transformation were carried out using rlog function in DESeq2 (85) (Version 1.32) R package.

Gene Set Variation Analysis: Gene Set Variation Analysis (86) (GSVA) is a non-parametric, unsupervised method for estimating variations in gene set enrichment among the samples of an expression dataset. The GSVA algorithm was implemented using the R Bioconductor open-source package gsva (version 1.40). GSVA was carried out in one of the following ways:

When individual datasets were analyzed, the preprocessed log 2 gene expression matrix of each dataset was used as the GSVA input. GSVA was run on each dataset separately. Before running GSVA, input genes were filtered and only those with interquartile range (IQR) of expression>0 across all the samples were considered for analysis. All analysis in FIGS. 29, 30, 35, 39, 43 and 45, are the result of this GSVA process. A minimum of 2 genes was required for each signature.

For the analysis of pooled nonlesional and control samples, log 2 gene expression values generated from independent preprocessing of all 16 datasets were concatenated to create a matrix whose rows consisted of 8425 genes detected across all datasets and whose columns consisted of the 1065 samples comprised of DLE, nonlesional (NL) DLE, ACLE, SCLE, PSO, NL PSO, AD, NL AD, SSc, and CTLs. Log2 values were then transformed to Z scores using scale( ) function in R. Z-score transformation converts each sample to have expression values with mean and unit variance equal to 0 (87, 88). This transformation permitted comparison of nonlesional disease samples to control directly. GSVA was then run on the following three inputs 1) 21 pooled nonlesional DLE and 168 pooled control samples, 2) 132 pooled nonlesional AD and 168 pooled control samples, and 3) 163 pooled nonlesional PSO and 168 pooled samples. The data presented in FIG. 49A is derived from this GSVA process. A minimum of 2 genes was required for each signature.

GSVA Gene Sets: The gene sets used for GSVA can be found in Table 4A-C Cellular pathway signatures: Gene sets employed in our GSVA analysis included 48 annotated and novel cellular and pathway signatures that have been implicated in lupus (4, 5, 6) or inflammatory skin diseases (7, 8). Immune cell gene sets were previously evaluated (21, 27) or amended slightly based upon data from the Human Protein Atlas (89, 90). Non-hematopoietic cell signatures were derived from the Human Protein Atlas (89, 90), previously published gene sets (91), and literature mining as previously described and employed. Pathway gene signatures were previously evaluated in lupus (21, 27), previously published (23, 24), or newly adopted by literature mining (92, 93). The output GSVA scores of each signature were used as features for training and validating ML classifiers. 40 of the 48 cellular and pathway gene signatures were used to implement the GSVA analysis on pooled nonlesional and control samples. The following signatures were excluded from this analysis because of insufficient gene numbers (</=2) in the 8,425 genes used: LDG, GC B cell, erythrocyte, IL1 cytokines, IL12 complex, IL21 complex, IL23 complex, and the immunoproteasome.

Keratinocyte signatures: 30 gene sets specific to keratinocytes treated with individual cytokines were created by from previously published studies (18, 94-100), (70, 1011-07). Only those genes that are upregulated in keratinocytes when treated with various cytokines were included in these sets.

T cell signatures: Gene sets for T cells were created from literature mining (108-111) and the Human Protein Atlas (89, 90) to distinguish seven different T cell subsets that have been implicated in inflammatory skin disease.

Classification and Regression Tree (CART): The library rpart (Version 4.1) was used to implement the CART algorithm for classification described previously (112, 113) and library rpart.plot (Version 3.1) was used to visualize classification trees. GSVA enrichment scores of cellular and pathway signatures were used as independent variables and specific disease(either DLE, PSO, AD, SSC, or CTL) was used as the dependent variable for analysis. Classification trees were built independently for each disease

### ML Analysis:

Creating input for ML: The input for ML was created by pooling GSVA enrichment scores of cellular and pathway gene signatures from multiple skin datasets based on classification of skin disease or sample (Table 5B). For every dataset, GSVA enrichment scores, that range from −1 to +1, were concatenated from multiple datasets, providing a sufficiently large cohort to train and validate various ML algorithms. 14 input data frames were created for 14 separate binary ML classifications (Table 5A). Seven of the 14 binary classifications involve comparing control samples (164 CTL) with either lesional samples (DLE, PSO, AD or SSc) or nonlesional samples (DLE, PSO or AD) of inflammatory skin diseases (Table 5A A-D and I-K), whereas the other six binary classifications involve comparing lesional DLE samples with lesional samples of other diseases (either PSO, AD or SSc) (Table 5A E-H) and nonlesional DLE samples with nonlesional samples of other diseases (Table 5A L-M). In addition, another binary classification consisted of comparing nonlesional PSO and nonlesional AD (Table 5A). For lesional skin classification, pooled samples were 90 DLE, 132 AD, 97 SSc, and 183 PSO. For nonlesional skin classification, pooled samples were 21 DLE, 163 PSO, and 132 AD, and for healthy skin pooled samples were 164 CTL (Table 5B).

Class balance strategies: Four class balance strategies, including: random undersampling (Table 5A C), random oversampling (Table 5A E, K) removing samples from an entire dataset (Table 5A F), and Synthetic Minority Oversampling Technique (SMOTE) (114) (Table 5A I, L, M) were used for classifications with class imbalance. The random undersampling strategy involves randomly selecting samples from the majority class, whereas the random oversampling strategy involves randomly duplicating examples from the minority class. SMOTE functions by randomly selecting samples from the minority class, finding its k nearest neighbors, randomly selecting a neighbor, and generating a synthetic sample at a randomly selected point between two samples in the feature space. As previously noted, we used random undersampling to trim the number of examples in the majority class then used SMOTE to oversample the minority class to balance class distribution. The purpose of all class balancing strategies was to have balanced representation of both classes for ML. The dataset was split into 70% training and 30% validation and class balancing strategies were applied on the training dataset. ML algorithms were then implemented, and evaluation matrices were noted. Receiver Operating Characteristic (ROC) curves and Precision-Recall (PR) curves were plotted using the matplotlib (Version3.3.4) library of Python. A ROC curve is graphical way to visualize trade-off between sensitivity and specificity. High area under the curve represents a low false-positive rate and a high true-positive rate. A PR curve is a measure of classification when classes are imbalanced. High area under the PR curve represents both high recall and high precision, where high precision relates to a low false-positive rate, and high recall relates to a low-false negative rate. For our analysis, we were interested in features that contributed the most towards separation of classes, hence RF was chosen as the primary ML classifier because it gives impurity-based feature importance. The top 15 features with decreasing Gini index from each classification were summarized in a bar graph using ggplots2 (Version 3.3.5) library in R. Capability of the top 15 features alone to separate the two respective classes was tested by repeating the 14 binary ML classifications using only the top 15 features. Various overlaps between the top 15 features of multiple classifications were visualized in Venn diagrams.

Binary ML classification: 14 separate binary ML classifications were carried out using scikit-learn (Version 0.24.1) library in Python (Version 3.8.2). For each binary classification, performance of several ML algorithms, including: Logistic regression (LR), K-Nearest Neighbor (KNN), Naïve Bayes (NB), Support Vector Machines (SVM), Random Forest (RF), and Gradient Boosting (GB) was evaluated based on sensitivity, specificity, Cohen kappa score, f-1 score, and accuracy. RF was chosen as the primary ML classifier because it gives impurity-based feature importance. The top 15 features with decreasing Gini index from each classification were summarized in a bar graph using ggplots2 (Version 3.3.5) library in R. Capability of the top 15 features to separate two respective classes was tested by repeating the 14 binary ML classifications using only the top 15 features.

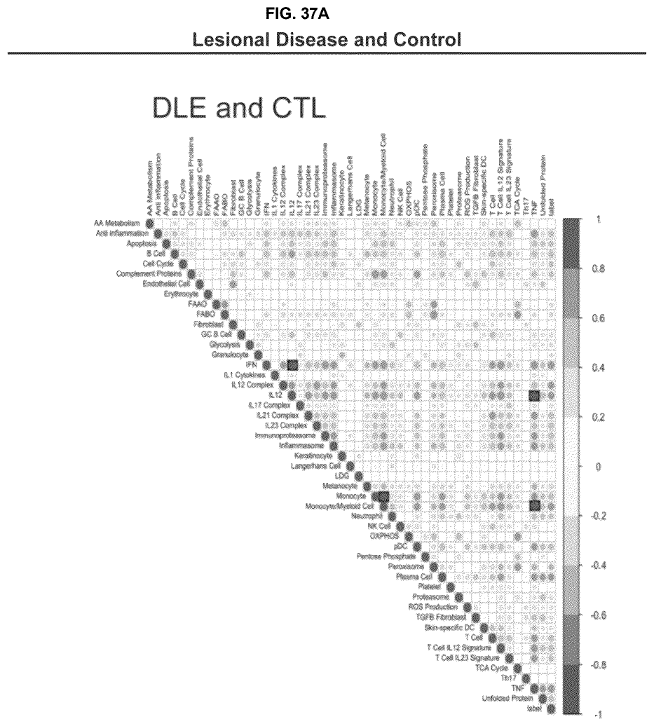

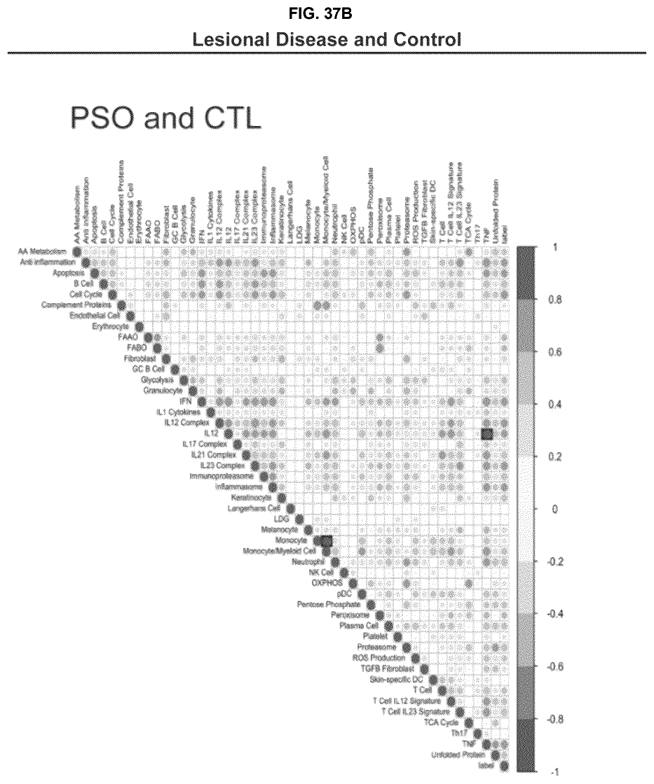

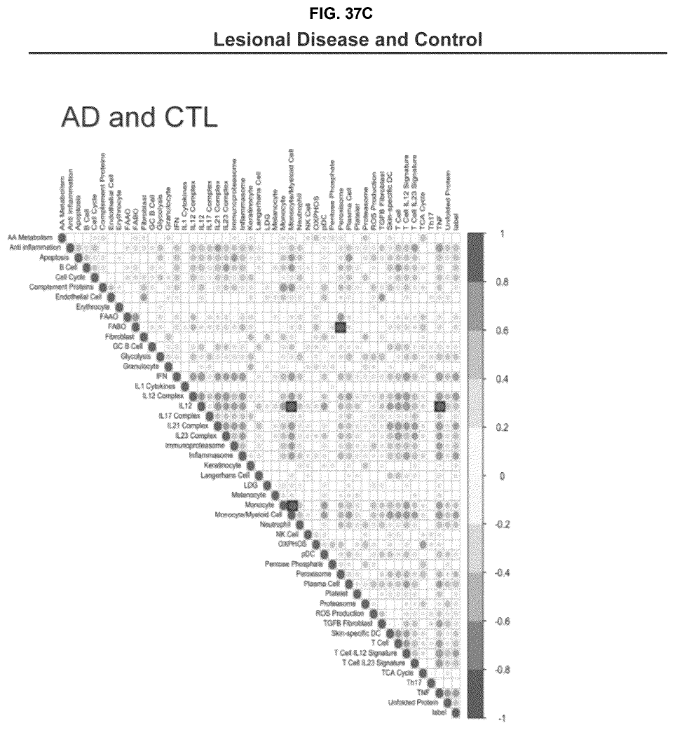

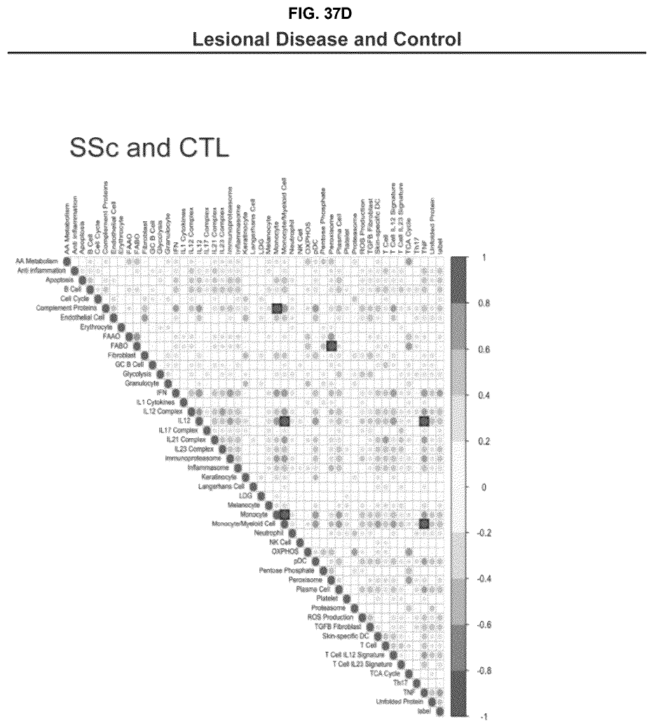

Feature correlation: Before carrying out binary ML classification, feature selection was necessary in order to remove noninformative or redundant features. We assessed feature redundancy by calculating the Pearson correlation between each feature and every other feature. Pearson correlation between features was computed using the cor function in R. corplot library in R was used to plot 22 Pearson correlation plots (FIG. 38, 42, 48, 51, 60). In 13 of these correlation plots, there was a pair of highly correlated features (correlation coefficient>0.8), and the feature with the lower correlation was removed using a greedy elimination approach; doing this allowed us to retain the most informative features for ML (Table 5A). Pearson correlation plots were also plotted for keratinocytesgene signatures and T cell signatures (FIG. 37, 46). High correlation between the keratinocyte gene signatures made them unsuitable for ML analysis (FIG. 36).

Statistical Analysis: Statistical differences between cohorts were evaluated using Welch's T test for lesional disease versus control GSVA scores from a single dataset, nonlesional samples versus control GSVA scores from combined dataset, mean Z-cores of nonlesional samples versus mean Z-scores of control samples of a single gene signature and Paired T test for lesional versus nonlesional comparison. The magnitude of this difference (the effect size) was estimated using Hedge's g(115) calculated as below

$$g = \frac{\overline{{x}_{1}} - \overline{{x}_{2}}}{\sqrt{\frac{( {n}_{1} - 1 ) * {s}_{1}^{2} + ( {n}_{2} - 1 ) * {s}_{2}^{2}}{{n}_{1} + {n}_{2} - 2}}}$$

where,

$$\overline{{x}_{1}} and \overline{{x}_{2}} = cohort 1 mean and cohort 2 mean respectively$$

$${n}_{1} and {n}_{2} = cohort 1 size and cohort 2 size respectively$$

$${s}_{1}^{2} and {s}_{2}^{2} = cohort 1 variance and cohort 2 variance respectively$$

cohort 1 and cohort 2 could be either disease and their respective control samples of a single dataset or nonlesional samples and control samples from combined dataset or mean z scores of nonlesional samples and mean z scores of control samples of a single gene signature or lesional and their paired nonlesional samples of a single dataset. All the statistical analysis was carried out in using effectSize (version 0.8.1) and stats (version 3.6.2) library in R.

Data Visualization: Heatmaps of GSVA Hedges' G effect size and violin plots of GSVA enrichment scores were visualized using GraphPad PRISM (Version 9.2.0). GSVA enrichment scores of gene signatures were visualized using violin plots in Prism or ComplexHeatmap (116) for hierarchical clustering (Version 2.8) package in R. Figures were made using AdobeIllustrator Creative Cloud (Version 25.3.1).

Data and materials availability: All transcriptomic data are previously published and available in the NCBI Gene Expression Omnibus (GSE52471, GSE72535, GSE81071, GSE109248, GSE100093, GSE120809, GSE117239, GSE117468, GSE130588, GSE137430, GSE157194, GSE121212, GSE95065, GSE58095, GSE130955) as seen in Table 3. All data are available in the main text or the supplementary materials. All bioinformatic software used in this publication is open source, freely available for R and Python. Additionally, example code used in this paper for GSVA, CART, and ML are available at figshare, www.figshare.com. File names are “AMPEL BioSolutions GSVA Code AFFY nonzeroIQR Code”, “AMPEL BioSolutions Stepwise and CARTCode”, “AMPEL BioSolutions ML Binary”.

TABLE 3
Publicly available datasets used

| | | | Samples used | Paired/ |
|---|---|---|---|---|
| GEO number | Reference | Platform | in this study | unpaired |
| GSE52471 | Jabbari et al. | [HG-U133A_2] | 3 PSO, 7 DLE, | Unpaired |
| | 2014 | Affymetrix | 3 CTL | |
| | 10.1038/ | Human Genome | | |
| | jid.2013.269 | U133A 2.0 Array | | |
| GSE72535 | Chong et al. 2015 | Illumina | 9 DLE, 8 CTL | Unpaired |
| | 10.1186/s13075- | HumanHT-12 | | |
| | 015-0839-3 | V4.0 expression | | |
| | | beadchip | | |
| GSE810171 | Liu et al. 2017 | [HuGene-2_1-st] | 26 + 21 DLE, | Unpaired |
| | 10.1002/art.40166; | Affymetrix | 23 + 20 SCLE, | |
| | Sarkar et al. | Human Gene 2.1 | 7 + 6 CTL | |
| | 2018 | ST Array | | |
| | 10.1136/annrheumdis- | [HuGene21st_Hs_ENTREZG_19.0.0] | | |
| | 2018-213197; | | | |
| | Tsoi et al. 2019 | | | |
| | 10.4049/ | | | |
| | jimmunol.1800650; | | | |
| | Billi et al. 2019 | | | |
| | 10.1172/ | | | |
| | jci.insight.127291 | | | |
| GSE109248 | Mande et al. | Illumina | 6DLE, 11SCLE, | Unpaired |
| | 2018 | HumanHT-12 | 17PSO, 13CTL | |
| | 10.1172/ | V4.0 expression | | |
| | JCI98219 | beadchip | | |
| GSE100093 | Werth et al. 2017 | [HT_HG- | 15L, 15NL (DLE) | Paired |
| | 10.1002/ | U133_Plus_PM] | | |
| | art.40052 | Affymetrix HT | | |
| | | HG-U133 + PM | | |
| | | Array Plate | | |
| GSE120809 | Barron et al. | [HG-U133A_2] | 6L, 6NL (DLE) | Paired |
| | 2019 | Affymetrix | | |
| | 10.4049/ | Human Genome | | |
| | jimmunol.1801209 | U133A Array | | |
| | | (HGU133A2 Hs | | |
| | | ENTREZG | | |
| | | 21.0.0) | | |
| GSE117239 | Brodmerkel et al. | [HG- | 63L, 63NL (PSO) | Paired |
| | 2019 | U133_Plus_2] | | |
| | 10.1016/ | Affymetrix | | |
| | j.jaci.2019.01.017 | Human Genome | | |
| | | U133 Plus 2.0 | | |
| | | Array | | |
| GSE117468 | Tomalin et al. | [HG- | 73L, 73NL (PSO) | Paired |
| | 2020 | U133_Plus_2] | | |
| | 10.1016/ | Affymetrix | | |
| | j.jaci.2019.10.041 | Human Genome | | |
| | | U133 Plus 2.0 | | |
| | | Array | | |
| GSE130588 | Guttman-Yassky | [HG- | 19L, 19NL (AD), | Paired and |
| | et al. 2019 | U133_Plus_2] | 20CTL | Unpaired both |
| | 10.1016/ | Affymetrix | | |
| | j.jaci.2018.08.022 | Human Genome | | |
| | | U133 Plus 2.0 | | |
| | | Array | | |
| GSE137430 | Ungar et al. 2021 | Illumina HiSeq | 38L, 38NL (AD) | Paired |
| | 10.1016/ | 4000 (Homo | | |
| | j.jaci.2020.04.055 | sapiens) | | |
| GSE157194 | Möbus et al. | Illumina HiSeq | 54L, 54NL (AD) | Paired |
| | 2021 | 3000 (Homo | | |
| | 10.1016/ | sapiens) | | |
| | j.jaci.2020.06.012 | | | |
| GSE121212 | Tsoi et al. 2019 | Illumina HiSeq | 27L & 27NL | Paired and |
| | 10.1016/ | 2500 (Homo | (PSO), 21L & | Unpaired both |
| | j.jid.2018.12.018 | sapiens) | 21NL (AD), | |
| | | | 38CTL | |
| GSE95065 | not published | [HG-U133A_2] | 18 SSc, 4 CTL | Unpaired |
| | | Affymetrix | | |
| | | Human Genome | | |
| | | U133A 2.0 Array | | |
| | | (HGU133A2 Hs | | |
| | | ENTREZG | | |
| | | 19.0.0) | | |
| GSE58095 | Assassi et al. | Illumina | 39 SSc, | Unpaired |
| | 2015 | HumanHT-12 | 36 CTL | |
| | 10.1002/art.39289 | V4.0 expression | | |
| | | beadchip | | |
| GSE130955 | Skaug et al. 2019 | Illumina HiSeq | 58 SSc, | Unpaired |
| | 10.1136/annrheu | 2500 (Homo | 33 CTL | |
| | mdis-2019- | sapiens) | | |
| | 215894 | | | |

| GEO number | Probes | After IQR |
|---|---|---|
| GSE52471 | 22215 | 10045 |
| GSE72535 | 47322 | 20762 |
| GSE810171 | 53617 | 26536 |
| GSE109248 | 47302 | 31415 |
| GSE100093 | 54613 | 20200 |
| GSE120809 | 22215 | 10072 |
| GSE117239 | 54613 | 14627 |
| GSE117468 | 54613 | 20200 |
| GSE130588 | 54613 | 15738 |
| GSE137430 | 18195 | 16594 |
| GSE157194 | 16873 | 15713 |
| GSE121212 | 15660 | 14696 |
| GSE95065 | 22215 | 9765 |
| GSE58095 | 47279 | 31399 |
| GSE130955 | 18972 | 16800 |

TABLE 4A-1
B Cell signature genes

AICDA, BANK1, BLK, BLNK, CD19, CD22, CD79A, CD79B,
CLEC17A, CR2, DAPP1, DTX1, FCRL1, FCRL2, FCRL3,
FCRL4, FCRL5, HLA-DOB, IGHD, IGHM, LY6D, MS4A1,
PAX5, POU2AF1, SH2B2, TNFRSF13B, TNFRSF13C, VPREB1,
VPREB3, ZBTB32, ZNF318

TABLE 4A-2
Endothelial Cell signature genes

DLC1, ECSCR, EMCN, FLT1, KDR, LDB2, LRRC32, MEIS2, PLAT,
PTPRB, SELE, TM4SF1, TM4SF18, VWF

TABLE 4A-3
Erythrocyte signature genes

BSG, GFI1B, GYPA, GYPB, GYPE, ICAM4, KEL, NFE2, RHD,
SLC4A1, TRIM10, TSPO2

TABLE 4A-4
Fibroblast signature genes

43894, ADAM33, ADAMTS6, AGTR1, ALPK2, ANGPTL2, ANKRD45, ANO2, ANPEP,
ARMC9, ASPN, BDKRB2, BDNF, BMPER, C11orf87, C1R, C1S, CATSPER3, CCDC102B,
CCDC80, CCDC81, CEMIP, CHAC1, CHRM2, CLMP, CNN1, COL14A1, COL3A1, COL5A1,
COLEC10, CPXM2, CPZ, CRABP2, CXCL12, DCN, DDR2, DKK1, DMRTA1, EGFL6,
ELOVL2, EMILIN1, FAM180A, FBLN7, FBN1, FGF5, FMN2, FOXF2, FST, FSTL1, GFRA1,
GLIS1, GLT8D2, GPR176, GREM1, GREM2, GRIK2, GUCY1A2, HSD17B2, HSPA2, HSPB3,
IL19, IQCD, KCNMB2, KIRREL3, KRT34, KRTAP1-5, KRTAP3-1, L1CAM, LAYN, LMOD1,
LOXL4, LUM, LY6K, MFAP4, MFAP5, MGARP, MGP, MKX, MMP2, MXRA5, MXRA8,
MYPN, NEXN, NFASC, NID2, NTF3, OLFML3, P3H3, P4HA3, PAMR1, PAX3, PCDHGA2,
PCDHGA3, PCDHGA7, PDE8B, PDGFRA, PDGFRL, PDZRN3, PLA2R1, PLEKHA4, PLPP4,
PLPPR4, PRKG1, PSG5, PTPRQ, PTRF, SEMA3A, SEMA5A, SEMA6D, SHOX, SLC16A2,
SMIM2, SPARCL1, SPHKAP, SSTR1, STC1, STXBP6, SUSD5, SVEP1, TBX15, THBS2,
TIMP2, TIMP3, TMEM119, TMEM130, TMEM47, TRHDE, TRPC4, UACA, UBL4B, VAT1L,
VEGFC, WNT5A, WNT5B, ZFPM2

TABLE 4A-5
GC B Cell signature genes

FCRLA, GCSAM, KLHL6, LRMP, NUGGC, RGS13

TABLE 4A-6
Granulocyte signature genes

CLC, HSH2D, MS4A2, PGLYRP1, PRG2, SYNE1

TABLE 4A-7
Keratinocyte signature genes

ABLIM, AKR1C1, ALDH8, ALOX12B, ANXA8, AQP3, ATDC,
BPAG1, CA12, CCND2, CD24, CDH3, CDKN1A, CDSN, COL17A1,
CST6, CSTA, DD96, DSC1, DSG1, DSP, EGFR, EVPL, FLG,
G0S2, GJA1, GLUL, GNA15, HBP17, IFI27, IFITM1, IGFBP7,
ITGA3, ITGB4, IVL, JUNB, JUP, KLK11, KLK7, KLK8, KRT1,
KRT14, KRT15, KRT16, KRT2A, KRT5, KRT6A, LAMA3, LGALS7,
LOR, NOTCH3, PPL, PRDX2, PRSS11, PRSS2, PRSS4, S100A2,
SERPINB2, SERPINB3, SERPINB4, SERPINB5, SERPINB7,
SERPINE1, SFN, SPINK5, SPRR1B, TACSTD2, TFAP2A, TGM1,
TP63, TUBA1, XP5

TABLE 4A-8
Langerhans Cell signature genes

CD1B, CD1C, CD1E, CD207

TABLE 4A-9
LDG signature genes

AZU1, CAMP, CEACAM3, CEACAM4, CEACAM6, CEACAM8,
CTSG, DEFA4, ELANE, MPO, OLFM4, RNASE3

TABLE 4A-10
Melanocyte signature genes

ASIP, CITED1, DCT, GPNMB, GPR143, MITF, MLANA, MLPH,
OCA2, PMEL, SLC24A5, SLC45A2, TYR, TYRP1

TABLE 4A-11
Monocyte signature genes

ADGRE1, C1QA, C1QB, C1QC, C2, CD14, CD300C, CD300E,
CD5L, CD68, CLEC5A, CSF1R, CYBB, FOLR2, LILRA1,
MARCO, MERTK, MS4A7, MSR1, SPIC

TABLE 4A-12
Monocyte/Myeloid Cell signature genes

ADGRE2, ADGRE3, AIF1, APOC1, BPI, BST1, C4A, C4B, C4BPA, C4BPB, C5, C6, C8A, C9,
CD163, CD1D, CD209, CD300LF, CD33, CFD, CFP, CHIT1, CLEC12A, CLEC12B, CLEC1A,
CLEC4A, CLEC4D, CLEC4E, CLEC4G, CLEC6A, CLEC7A, CRISP3, CSF2RA, CSF2RB,
CST3, CTSS, F12, FCER1A, FCER1G, FCGR1A, FCGR1B, FCGR2A, FCGR2C, FLT3, GRN,
IGSF6, ITGAX, LGALS12, LGALS4, LGALS9, LILRA2, LILRA5, LILRA6, LILRB2, LY86,
LYVE1, LYZ, MEFV, MMP8, MNDA, MPEG1, MS4A4A, MS4A6A, NLRP12, NLRP3, NOD2,
OLR1, OSCAR, OSM, PILRA, PRAM1, RETN, S100A12, S100A8, S100A9, SCARB1,
SECTM1, SEMA4A, SERPING1, SGK1, SIGLEC1, SIGLEC10, SIGLEC14, SIGLEC5,
SLC11A1, SLITRK4, SMPDL3B, SPI1, TEK, THBD, TLR2, TLR8, TNFSF13B, TREM1,
TREML4, TYROBP, VENTX, VSIG4, VSTM1

TABLE 4A-13
Neutrophil signature genes

ARG1, BMX, CD177, CSF3R, DEFA1, DEFA1B, DEFA3,
DEFB103A, DEFB103B, DEFB106B, DEFB136, DEFB4A,
FPR2, OR1J2, PRTN3, SLC2A3, SLPI

TABLE 4A-14
NK Cell signature genes

KIR2DL4, KLRC3, KLRF1, NCAMI, NCR1, SH2D1B, TNFSF11, TXK

TABLE 4A-15
pDC signature genes

CLEC4C, IRF7, LILRA4, NRP1, PACSIN1, PLA2G5, PLAC8, PTCRA,
SERPINF1, SLC15A4, TCF4

TABLE 4A-16
Plasma Cell signature genes

CD38, CRELD2, ELL2, FKBP11, IGKV4-1, IGLV2-14, ITM2C,
JCHAIN, MANF, MZB1, PDIA4, PRDX4, SDF2L1, SPATS2,
TNFRSF17, UAP1

TABLE 4A-17
Platelet signature genes

GP1BA, GP5, GP6, GP9, PF4, PF4V1, PLEK, PPBP, SLC35D3

TABLE 4A-18
Skin-specific DC signature genes

CLEC10A, CLEC9A, GPR31, MRC1, XCR1

TABLE 4A-19
T- Cell signature genes

BCL11B, CAMK4, CD28, CD3D, CD3G, CD5, CD6, GPR171, ITK,
RGCC, TESPA1, THEMIS, TRAT1

TABLE 4A-20
Th17 signature genes

CCR6, CXCR3, IL17A, IL17F, IL22, IL26, KLRB1, RORA, RORC

TABLE 4B-1
AA Metabolism signature genes

AASS, AGXT, AGXT2, ALDH4A1, ALDH7A1, ARG1, GAD1, GLUD1,
GOT1, GPT2, GRHPR, IVD, MCCC2, OAT, OTC, OXCT1, PC, PSAT1,
SDS, IL1RN, SOCS3, TNFAIP3

TABLE 4B-2
Apoptosis signature genes

AIFM1, BAD, BAK1, BAX, BID, CASP10, CASP3, CASP6, CASP7,
CASP8, CASP9, ENDOG, FAS, HTRA2, TNFRSF1A

TABLE 4B-3
Cell Cycle signature genes

ASPM, AURKA, AURKB, BRCA1, CCNB1, CCNB2, CCNE1, CDC20,
CENPM, CEP55, E2F3, GINS2, MCM10, MCM2, MKI67, NCAPG,
NDC80, PTTG1, TYMS

TABLE 4B-4
Complement Proteins signature genes

C1QA, C1QB, C1QC, C1R, C1S, C2, C3, C4A, C4B, C5, C6, C7, C8A,
C8B, C8G, C9

TABLE 4B-5
FAAO signature genes

HAAO, HACL1, PEX13, PHYH, SLC27A2

TABLE 4B-6
FABO signature genes

ABCD1, ABCD2, ABCD3, ACAA2, ACACB, ACAD11, ACADL,
ACADM, ACADS, ACADVL, ACAT1, ACAT2, ACOX1, ACOX2,
ACOX3, ACOXL, ACSBG2, ACSL5, ADIPOQ, AKT2, AUH, BDH2,
CPT1A, CPT2, CROT, DECR1, ECHDC1, ECHDC2, ECHS1, ECI1,
ECI2, EHHADH, ETFA, ETFB, ETFDH, FABP1, GCDH, HADH,
HADHA, HADHB, HIBCH, HSD17B4, IRS1, IRS2, IVD, LEP, PEX2,
PEX5, PEX7, SESN2, SLC25A17, SLC27A2, TWIST1

TABLE 4B-7
Glycolysis signature genes

ALDOA, ALDOB, ALDOC, ENO1, ENO2, ENO3, GAPDH, GCK, GPI,
HK1, HK2, HK3, HKDC1, LDHA, LDHAL6A, LDHAL6B, LDHB,
LDHC, PFKFB1, PFKFB2, PFKFB3, PFKFB4, PFKL, PFKM, PFKP,
PGAM1, PGAM2, PGK1, PGK2, PKM, SLC2A1, SLC2A3, SLC2A4,
TPI1

TABLE 4B-8
IFN signature genes

EIF2AK2, GBP1, GBP2, GBP4, HERC5, HERC6, IFI27, IFI30, IFI35,
IFI44, IFI44L, IFI6, IFIT1, IFIT2, IFIT3, IFIT5, IFITM1, IFITM2,
IFITM3, ISG15, ISG20, MX1, MX2, OAS1, OAS2, OAS3, OASL,
RSAD2, SAMD9, SAMD9L, SP100, SP110

TABLE 4B-9
IL1 Cytokines signature genes

IL18, IL1B

TABLE 4B-10
IL12 Complex signature genes

IL12A, IL12B, IL12RB1, IL12RB2

TABLE 4B-11
IL12 signature genes

ACLY, AKAP10, APOL3, BACH2, BRCA2, CALD1, CASK, CASP1,
CCR5, CDKN3, CXCL10, CXCL9, CYBB, DEFA1, ETAA1, FASLG,
FBXL2, FCER2, FCGR1A, GBP1, GBP2, GLS, GNPDA1, GSTM5,
GZMB, HHEX, HP, HSPA6, IFNG, IL16, IL18BP, IL18R1, IL1A,
INPP5D, INSIG1, IRF1, KLF2, KRT8, LIMK1, LINC00597, LY75,
MMP25, NIN, NLRP1, PCDH9, SELL, SERPIND1, SLAMF1, SOCS1,
STAT1, TAP2, TBX21, TFF1, TNFAIP2, TNFAIP3, TNFSF10, TXK

TABLE 4B-12
IL17 Complex signature genes

IL17A, IL17F, IL17RA, IL17RC, TRAF3, TRAF3IP2

TABLE 4B-13
IL21 Complex signature genes

IL21, IL21R, IL2RG

TABLE 4B-14
IL23 Complex signature genes

IL12B, IL12RB1, IL23A, IL23R

TABLE 4B-15
Immunoproteasome signature genes

PSMB10, PSMB8, PSMB9

TABLE 4B-16
Inflammasome signature genes

AIM2, CASP1, CASP5, CTSB, GSDMB, GSDMD, NAIP, NEK7,
NLRC4, NLRP1, NLRP3, NOD2, P2RX7, PANX1, PYCARD, RIPK1

TABLE 4B-17
OXPHOS signature genes

ATP5A1, ATP5B, ATP5D, ATP5E, ATP5F1, ATP5G1, ATP5G2,
ATP5G3, ATP5H, ATP5I, ATP5J, ATP5J2, ATP5L, ATP5O, ATP5S,
BCS1L, CEP89, COA1, COA3, COA4, COA5, COA6, COA7, COX10,
COX10-AS1, COX11, COX14, COX15, COX16, COX17, COX18,
COX19, COX20, COX4I1, COX4I2, COX5A, COX5B, COX6A1,
COX6A2, COX6B1, COX6B2, COX6C, COX7A1, COX7A2,
COX7A2L, COX7B, COX7B2, COX7C, COX8A, COX8C, CYC1,
CYCS, DNAJC15, MT-ATP6, MT-ATP8, MT-CO1, MT-CO2, MT-CO3,
MT-CYB, MT-ND1, MT-ND2, MT-ND3, MT-ND4, MT-ND4L,
MT-ND5, MT-ND6, NDUFA1, NDUFA10, NDUFA11, NDUFA12,
NDUFA13, NDUFA2, NDUFA3, NDUFA4, NDUFA4L2, NDUFA5,
NDUFA6, NDUFA7, NDUFA8, NDUFA9, NDUFAB1, NDUFAF1,
NDUFAF2, NDUFAF3, NDUFAF4, NDUFAF5, NDUFAF6, NDUFAF7,
NDUFAF8, NDUFB1, NDUFB10, NDUFB11, NDUFB2, NDUFB2-AS1,
NDUFB3, NDUFB4, NDUFB5, NDUFB6, NDUFB7, NDUFB8,
NDUFB9, NDUFC1, NDUFC2, NDUFS1, NDUFS2, NDUFS3, NDUFS4,
NDUFS5, NDUFS6, NDUFS7, NDUFS8, NDUFV1, NDUFV2,
NDUFV3, NUBPL, OXA1L, RFESD, SCO1, SCO2, SLC25A4, SURF1,
TACO1, TIMMDC1, TMEM126B, TRAP1, TTC19, UQCC1, UQCC2,
UQCC3, UQCR10, UQCR11, UQCRB, UQCRC1, UQCRC2, UQCRFS1,
UQCRH, UQCRHL, UQCRQ

TABLE 4B-18
Pentose Phosphate signature genes

G6PD, H6PD, PGD, PRPS1, PRPS1L1, PRPS2, RBKS, RGN, RPE,
RPIA, TALDO1, TKT, TKTL1, TKTL2

TABLE 4B-19
Peroxisome signature genes

ABCD3, ACAA1, ACOX1, ACOX2, ACOX3, CAT, DDO, DECR2,
EHHADH, HAO2, HMGCL, HSDL2, ISOC1, KXD1, PAOX, PEX1,
PEX10, PEX11A, PEX12, PEX16, PEX19, PEX26, PEX3, PEX5,
PEX6, PEX7, PHYH, PIPOX, PMVK, PXMP2, SCP2, SLC25A17

TABLE 4B-20
Proteasome signature genes

ADRM1, NGLY1, PAAF1, POMP, PSMA1, PSMA2, PSMA3,
PSMA3-AS1, PSMA4, PSMA5, PSMA6, PSMA7, PSMA8, PSMB1,
PSMB11, PSMB2, PSMB3, PSMB4, PSMB5, PSMB6, PSMB7,
PSMC1, PSMC2, PSMC3, PSMC4, PSMC5, PSMC6, PSMD1,
PSMD10, PSMD11, PSMD12, PSMD13, PSMD14, PSMD2,
PSMD3, PSMD4, PSMD5, PSMD5-AS1, PSMD6, PSMD6-AS2,
PSMD7, PSMD8, PSMD9, PSME1, PSME2, PSME3, PSME4,
PSMF1, PSMG1, PSMG2, PSMG3, PSMG3-AS1, PSMG4,
RAD23B, SHFM1, UBLCP1, UBQLN1, UCHL5, ZFAND2A

TABLE 4B-21
ROS Production signature genes

GPX1, GPX3

TABLE 4B-22
T Cell IL12 signature genes

CCL5, CXCR1, GZMH, IFNG, IL12RB2, ILIRL1, LIFR,
TNFSF10, TNFSF13B

TABLE 4B-23
T Cell IL23 signature genes

CCL17, CCL20, CCL22, CCL7, CCR1, CSF2, CXCL2, CXCR5, IL17A,
IL17F, IL17RC, IL1R1, IL23R, IL6, ITGA3, PDGFB, TNF

TABLE 4B-24
TCA cycle signature genes

ACO2, CS, DLAT, DLD, DLST, FH, GLUD1, IDH1, IDH2, IDH3A,
IDH3B, IDH3G, MDH2, MPC1, MPC2, OGDH, OGDHL, PDHA1,
PDHA2, PDHB, PDHX, PDK1, PDK2, PDK3, PDK4, PDP1, PDP2,
PDPR, SDHA, SDHAF1, SDHAF2, SDHAF3, SDHAF4, SDHB,
SDHC, SDHD, SUCLA2, SUCLG1, SUCLG2, SUGCT

TABLE 4B-25
TNF signature genes

ACLY, ACSL1, ADGRE2, AK3, AKAP10, AMPD3, APOL3, ARID3A, ARSE, ASAP1,
B4GALT5, BCL2A1, BHLHE41, BHMT, BIRC3, BRCA1, CALD1, CASP1,
CASP10, CCL15, CCL20, CCL23, CCL3L1, CD37, CD38, CD83, CDKN3, CKB, CR2,
CTNND2, CXCL1, CXCL2, CXCL3, CXCL8, CYP27B1, DAB2, EBI3, EGR1,
EGR2, EPB41, EREG, ETAA1, F3, FABP1, FBXL2, FCER2, FCGR2A, FLJ11129, FLNA,
G0S2, GBP1, GCH1, GJB2, GLS, GMIP, GP1BA, GRK3, HCAR3,
HHEX, HOMER2, HP, ICAM1, IDO1, IFI44, IKBKG, IL16, IL18, ILIA, IL1B, IL1RN, IL6,
INHBA, INSIG1, ITGA6, KITLG, KLF1, KMO, LGALS3BP, MAP3K4,
MARCKS, MGLL, MMP19, MN1, MRPS15, MSC, MTF1, MX1, NAMPT, NELL2, NFKB1,
NFKB2, NFKBIA, NFKBIZ, NKX3-2, NR3C1, OAS3, PATJ, PDE4DIP,
PDPN, PIAS4, PLAUR, PTGES, PTGS2, RELB, RPGR, RPS9, SDC4, SERPIND1, SFRP1,
SH3BP5, SLAMF1, SLC30A4, SOD2, SPI1, SSPN, STAT4, TAF15,
TAP2, TBX3, TFF1, TNF, TNFAIP2, TNFAIP3, TNFRSF11A, TRAF1, TSC22D1, TYROBP,
UBE2C, VEGFA, WT1

TABLE 4B-26
Unfolded Protein signature genes

B4GALT3, CALR, CALU, CANX, CDS2, CHST12, CHST2, DERL1, DERL2, DNAJC3,
EDEM2, EDEM3, EMC9, ERAP1, ERGIC2, ERO1L, EXT1, GALNT2,
GOLT1B, HERPUD1, HYOU1, IER3IP1, IMPAD1, KDELC1, KDELR2, LMAN2, LPGAT1,
MAN1A1, MANEA, MANF, NUCB2, PDIA4, PDIA6, PIGK, PPIB, SEC24D,
SEC61G, SPCS3, SSR1, SSR3, TRAM1, TRAM2, UGGT1, XBP1

TABLE 4B-27
TGFB Fibroblast signature genes

ABTB2, ACOXI, ACTA2, ACTC, ACTN1, ACTN3, ADAM12, ADAM19, ADAMTS4,
ADCY7, AK3, ALS2CR4, AMIGO2, ANGPTL4, AQP1, ARK5, ARL4A, ARNTL,
ASE-1, ASNS, ATOH8, ATP10A, ATPIB1, AVP, AXUD1, B4GALT1, BAG3, BFAR,
BHLHB2, BLOC1S2, BM039, BMP6, BMPR2, C10orf22, C10orf30, C14orf138,
C14orf31, C16orf30, C18orf1, C20orf139, C20orf39, C21orf93, C5orf13, C6orf145, C6orf85,
C9orf19, C9orf3, C9orf62, CALM2, CARD4, CBFB, CCDC8, CCL2,
CDH2, CDKN2B, CEBPA, CH25H, CHIC2, CHST11, CHST5, CHSY1, CLC, CMKOR1, CNN3,
COL4A1, COL4A2, COL5A1, COL5A2, COMP, CREB3L2, CRLF1,
CRY1, CSRP1, CSRP2, CTGF, CTPS, CXCL12, CXXC5, CYR61, DACT1, DDIT4, DLC1,
DLX2, DNAJB4, DNAJB5, DNAJB9, DOK5L, DSP, DTR, DUSP1,
DYRK2, E2F7, EIF4EBP1, ELN, ENC1, ENPP1, EPHB3, ERN1, EYA2, FBXO32, FGF18,
FGF2, FGFR3, FGFRL1, FHL2, FLJ10350, FLJ10357, FLJ10378,
FLJ14054, FLJ20364, FLJ20366, FLJ20701, FLJ22938, FLJ39370, FLJ45248, FN5, FOXP1,
FSTL3, FUS, FZD8, GABRE, GADD45B, GARS, GAS7, GATA6,
GDF15, GDF6, GEM, GLS, GNPNAT1, GOPC, GPAM, GPR68, GPT2, GSTT2, HCMOGT-1,
HES1, HIF1A, HILS1, HNRPAB, HNRPK, HOMER1, HOXB2,
HOXC8, HSPA5, HSPB7, HSXIAPAF1, ID1, ID3, ID4, IER3, IER5L, IGF1, IL11, IL21R, IL4R,
IL6, ITR, IVNS1ABP, JUNB, K-ALPHA-1, KCNE4, KCNG1, KCNK1,
KCNN4, KCNS3, KCTD11, KIAA0033, KIAA0280, KIAA1102, KIAA1644, KIAA1754,
KLF10, KLF13, KLF2, LDHA, LHFPL2, LIF, LIM, LIMKI, LIMK2, LIMS3,
LMCD1, LMO4, LOC222171, LOC283824, LOC284454, LOC339047, LOC440502, LOC51333,
LRIG1, LRRC8, LTBP2, MAP3K2, MBD4, MGC14376, MGC15476,
MGC16121, MGC29875, MGC4504, MGC45871, MGC8685, MGLL, MICAL2, MICAL-L1,
MIR100HG, MONDOA, MRC2, MSX1, MTCH1, MTHFD2, NEDD4,
NEDD9, NET1, NFATC1, NFYC, NGEF, NID67, NKD2, NLF1, NNMT, NP, NPAS1, NPTX1,
NRBF2, NRG1, NUP98, ODC1, P4HA2, P4HA3, PACSIN2, PAWR,
PDGFA, PDLIM4, PFKP, PGK1, PGM2L1, PGM3, PHF17, PHLDA2, PHLDB1, PICALM,
PIM1, PITX2, PKM2, PLAU, PLAUR, PLEKHA1, PLK3, PLOD2, PNMA1,
PODXL, POFUT2, PPP1R13L, PPP1R14C, PPP1R3B, PRICKLE2, PRKAB2, PRO1855, PRPS1,
PRPS1L1, PRRX2, PSAT1, PTDSR, PTPNS1, RAI14, RAI17,
RASL11B, RGS3, RKHD3, RNF126, RPL21, RPL5, RTTN, RUNX1, RUNX2, RUSC2,
S100A16, SAMD11, SARS, SCD, SCHIP1, SDFR1, SERP1, SERPINE1,
SERTAD1, SERTAD4, SGCG, SGK, SH3MD1, SIAT4A, SKIL, SLC10A3, SLC16A3,
SLC19A2, SLC1A5, SLC20A1, SLC26A1, SLC2A1, SLC38A5, SLC39A14,
SLC4A2, SLC7A11, SLC7A5, SMAD7, SMARCB1, SNAI1, SNF1LK, SNX24, SOX4, SOX9,
SPARC, SPHK1, SRF, STC2, STCH, STK38L, SYNJ2, SYVN1, TBX3,
TD-60, TES, TGFB1, TGFBR1, TGM2, TIMP3, TIPARP, TMEPA1, TMPO, TNC, TNFRSF12A,
TNFRSF19L, TPM1, TRIB1, TRIB2, TRIB3, TSK, TUBA3, TUBA6,
TUBB2, TUBB3, TUBB4, TUBB6, TUFT1, UAP1, UCK2, UGDH, ULK1, UNC5B, UPP1,
USP35, VEGF, VLDLR, VMP1, WNT5B, XBP1, ZNF281, ZNF336, ZNF469, ZNF537

TABLE 4B-28
Anti-inflammation signature genes

IL1RN, SOCS3, TNFAIP3

TABLE 4C
Genes within T cell signatures.

| T cell Signature | Genes in the signature |
|---|---|
| Dermal Aner/Act T Cell | CD160, CTLA4, ICOS, KLRG1, LAG3, |
| | PDCD1 |
| Dermal CD8 T Cell | CD8A, CD8B |
| Dermal Tfh | BTLA, IL21, SH2D1A |
| Dermal Th1 | CCL5, CXCR3, EOMES, IFNG, PRF1, |
| | TBX21, GZMK |
| Dermal Th17 | CCR6, IL12RB1, IL17A, IL17F, |
| | IL22, IL23R, IL26, KLRB1, RORC |
| Dermal Th2 | GATA3, IL13, IL4, IL4R, IL5 |
| Dermal Treg | FOXP3, IKZF2, TNFRSF9 |

TABLE 4D
Genes within keratinocyte signatures

| Keratinocyte | | Reference |
|---|---|---|
| signature | Genes in the signature | number |
| IFNa2a | IFIT1, IFIT2, IFIT3, RSAD2, ISG15, MX1, DDX58, IFI44, | Yao et al. |
| | PARP9, IFIH1, LAMP3, USP18, IFI44L, IFITM1, STAT1, | 2008 [100] |
| | OAS2, GBP1, PLSCR1, DTX3L, SP110, IFIT5, HERC5, | |
| | IRF7, CXCL10, DDX60L, HERC6, TRIM21, EPSTI1, MX2, | |
| | NLRC5, HELZ2, OAS1, DDX60 | |
| IFNb | IFIT1, IFIT2, IFIT3, RSAD2, ISG15, MX1, IFI44, PARP9, | Yao et al. |
| | IFIH1, USP18, LAMP3, CXCL10, OAS2, DDX58, STAT1, | 2008 [100] |
| | IFI44L, GBP1, DTX3L, IFITM1, IFIT5, NLRC5, IRF7, MX2, | |
| | DDX60L, HERC5, HERC6, TRIM21, XAF1, EPSTI1, | |
| | PLSCR1, CH25H, PARP12, | |
| | SP110, SAMD9 | |
| IFNg_A | CXCL10, CXCL9, UBD, CXCL11, C1S, HLA-DRA, C1R, | Nograles et al. |
| | HLA-DRB5, PLAAT4, CCL2, RSAD2, HLA-DMA, PSMB9, | 2008 [18] |
| | APOL3, GBP2, CD74, HLA-DRB1, ICAM1, ISG20, BST2, | |
| | IRF1, CTSS, SERPING1, AIM2, IDO1, APOL1, HLA-DPA1, | |
| | GLDC, IL32, | |
| | RARRES1, SECTM1, ETV7, APOL6, IL15, GBP1, IFI35, | |
| | HLA-DRB4, IFIT3, XAF1, CCL8, CFB, CEACAM1, CFH | |
| IFNg_B | S100A7, S100A8, MMP1, MMP3, KLK7, KRTDAP, KRT10 | Wolk et al. |
| | | 2006 [96] |
| IFNg_C | CXCL10, PARP9, NLRC5, GBP1, PSMB9, CXCL9, IRF1, | Yao et al. |
| | DTX3L, PSMB8, IFIT5, TAP1, TRIM69, SERPING1, | 2008 [100] |
| | SECTM1, WARS1, | |
| | RAD50, INPP1, MCUB, BTN3A3, TLDC2, APOL6, SP110, | |
| | EDNRA, CASP7, NMI, IFIH1, IFIT3, IFIT2, APOL2, | |
| | BTN3A2, GBP2, DDX58, STAT1, CACHD1, TRIM21, IRF9 | |
| IFNg_D | STAT1, MX2, MX1, ISG15, IRF7, IFI6 | Mansourian et |
| | | al. 2004 [94] |
| IFN | IFIT1, IFIT3, IFIT2, ISG15, RSAD2, MX1, IFI44, PARP9, | Yao et al. |
| | IFIH1, LAMP3, USP18, STAT1, OAS2, DDX58, DTX3L, | 2008 [100] |
| | IFITM1, IFIT5, IRF7, IFI44L, PLSCR1, HERC6, TRIM21, | |
| | IRF9, GBP1, PARP12, EIF2AK2, SP110, DDX60L, HERC5, | |
| | HELZ2, OAS1, ZC3HAV1, | |
| | EPSTI1, NLRC5, TRIM14 | |
| IL1 | SERPINB4, SERPINB3, SLC6A14, TGFA, HEPHL1 | Li et al. 2014 |
| | | [107] |
| IL1a | SERPINB4, SERPINB3, SLC6A14, TGFA, HEPHL1 | Li et al. 2014 |
| | | [107] |
| IL1b | NCF2, SAA2, C3, GBP1, IFIT2, IFIT3, IRF1, PRDM1, CFB, | Yano et al. |
| | CTSC, SERPINB1, SERPINB3, SERPINB4, MMP9, PLAT, | 2008 [99] |
| | SERPINA3, PSMB9, CCL20, CCL5, CXCL1, CXCL2, | |
| | CXCL3, TYMP, EDN1, IL1A, IL1B, IL36G, CXCL8, | |
| | INHBA, TGFA, TNFAIP2, MPZL2, NINJ1, IL32, TNFAIP6, | |
| | CLDN4, KRT34, S100A12, S100A7, S100A8, S100A9, | |
| | SPRR2B, PDZK1IP1, RHCG, PTP4A3, LYN, PLAU, SGK1, | |
| | HCAR3, IFNGR1, IL1RN, HBEGF, PLAUR, PPIF, NDRG2, | |
| | NFKB1, NFKB2, NFKBIA, NFKBIE, RELB, WNT4, NOD2, | |
| | RIPK2, TNFSF10, BIRC3, IER3, TNFAIP3, PPP1R15A, | |
| | RCAN1, BHLH340, MAFF, REL, TNIP1, ZFP36, ALDH1A3, | |
| | APOBEC3A, OAS3, EPHX3, CYB5R2, SOD2, TAPBP, | |
| | ATP1B1, CIQTNF1, LCN2, SLC11A2, SLC6A14, TFRC | |
| IL17_A | SPRR2A, DEFB4A, IL36G, SPRR2C, SPRR2E, SPRR2F, | Chiricozzi et |
| | NFKBIZ, ZC3H12A, PI3, LCN2, SERPINB4, SPRR2B, | al. 2011 [102] |
| | CXCL8, S100A8, S100A9, SPRR2D, NDRG2, SAA1, RHCG, | |
| | IL1B, ATP1B1, IER3, CCL20, PDZK1IP1, ALDH1A3, | |
| | CXCL2, C15orf48, NOD2, ALOX12B, ARHGEF37, SOX7, | |
| | SLC11A2, ANKK1, NFKBIA, GJB6, TYMP, SAT1, MAFF, | |
| | DNER, ISG20L2, RBM14, F2R, | |
| | ACTA2, TNNI2, CPA4 | |
| IL17_B | CXCL1, CXCL2, CXCL5, CXCL6, CXCL8, CXCL9, | Shen et al. |
| | CXCL10, CXCL11, CCL2, CCL5, CCL7, CCL11, CXCL12, | 2008 [98] |
| | CCL20, IL6, IL19, CSF2, CSF3, ICAM1, PTGS2, NOS2, | |
| | LCN2, DEFB4A, S100A7, S100A8, S100A9, MUC5AC, | |
| | MUC5B, EREG, SOCS3, | |
| | TNFSF11, MMP1, MMP3, MMP9, MMP13, TIMP1, | |
| | ADAMTS4, CEBPB, CEBPD, NFKBIZ | |
| IL17_C | DEFB4A, S100A7, S100A12, CCL20, SERPINB4, CXCL6, | Nograles et al. |
| | PDZK1IP1, SAA2, SPRR2C, ZC3H12A, IL36G, CXCL8, | 2008 [18] |
| | SLC6A14, | |
| | CXCL1, CXCL2, RHCG, CXCL5, NDRG2, NOD2, LCN2, | |
| | PI3, C3, CXCL3, MAP3K8, SPRR2D, IL1B, CRABP2, NMB, | |
| | SLC11A2, S100A9, SLC25A12, TFRC, NAV3, CYB5R2, | |
| | IER3, SAT1, RPS6KA5, NAMPT, S100A8, ALDH1A3, | |
| | TYW1 | |
| IL17a_D | Clorf68, LY6G6C, LCE1B, NIPAL4, SLC46A2, SERPINB4, | Li et al. 2014 |
| | SERPINB3, SLC6A14, TGFA, HEPHL1 | [107] |
| IL17_TNFa | IL19, CXCL8, IL23A, CCL20, IL6, CXCL1, TNF, IL17C, | Chiricozzi et |
| | CXCL5, DEFB4A, CFB, S100A7A, S100A7, PLAT, IL36G, | al. 2011 [102] |
| | CXCL6, IL1B, | |
| | IL36RN, TNFSF18, PI3, LCN2, S100A9, MAP3K8, S100P, | |
| | S100A8 | |
| IL17a_TNF | CNFN, TREX2, SPRR2B, SPRR2D, SULT2B1 | Li et al. 2014 |
| | | [107] |
| IL17c_3 hr | CSF3, CXCL8, CXCL1, CXCL2, CXCL3, IL1B, IL36G, | Ramirez- |
| | CCL2, CCL20, IL17D, NFKBID, NFKBIZ, TNFAIP6, | Carrozzi et al. |
| | IL1RL1, MMP12 | 2011 [105] |
| IL17c_24 hr | CSF3, CXCL1, IL1B, IL36G, CCL20, IL17D, C3, NFKBID, | Ramirez- |
| | NFKBIZ, TNFAIP6, TNIP3, IL1RL1, MMP12, SAA4, VNN3, | Carrozzi et al. |
| | S100A7, | 2011 [105] |
| | S100A7A, S100A8, S100A9, DEFB1, DEFB4A | |
| IL22_A | S100A7, SERPINB4, S100P, SERPINB1, CARHSP1, HRH1, | Nograles et al. |
| | CYP27B1, TM4SF1, CASP4, RAB27A, IL13RA1 | 2008 [18] |
| IL22_B | S100A7, S100A8, MMP1, MMP3 | Wolk et al. |
| | | 2006 [96] |
| IL4_IL13 | HSD3B1, NTRK1, CLDN5, CCL26, NNMT, LOXL4, TNC, | Hirakawa et |
| | SERPINB3, SERPINB4, PDZK1IP1, KCNJ12, CA2, | al. 2011 [104] |
| | HSD3B2, RFLNA, IL33, MGAM, ST8SIA1, CXCL6, | |
| | ANOS1, SIDT1, CAPN14, CISH, CYP1B1, TOX, P2RY1, | |
| | MYH15, ID3, LSP1, CSF3, GJB2, GKN1, LRRC8B, | |
| | TREML2, OGDHL, CTSC, PALMD, GUCA1C, SLCO2A1, | |
| | BRINP1, E2F2, MYB, NPNT, NR3C2, PPFIBP2, EDNRA, | |
| | GDPD2, SERPINB13, HAS3, BATF3, MMP7, CPM, APOL3, | |
| | RGS16, MEST, MYOZ2, CENPV, STAR, KIF24, CRABP2, | |
| | GJB6, TMEM45B, TENM2, MAP2K6, PCDH7, NRG1, | |
| | SLC5A12, KLRG1, CFH, GLS2, KITLG, SLC7A4, BTBD3, | |
| | NMU, POU2F3, RASL11B, KRT24, KLHL23, TF, HPDL, | |
| | SLC2A5, MAN1C1, ZNF367, CFHR1, KIRREL3, NEFH, | |
| | MMP12, DUSP19, PHGDH, VSNL1, LRFN2, KIAA1958, | |
| | JADE1, DUOX1, FABP6, KCNRG, SEMA3A, PNPT1, | |
| | C10orf82, RTP5, UFD1, NABP1, NPW, IL7R, TP73, HLA- | |
| | DOA, CTRL, DESI2, RTKN2, FOLH1, SIPA1L2, DUSP13, | |
| | SLC15A2, FJX1, IL15RA, GATA3, MAP3K14, KRT8, | |
| | KRT18P45, TP53AIP1, SHF, TPK1, HSPD1, FZD10-AS1, | |
| | ZNF697, CRACR2A, BLMH, SLC35G1, PRDM13, ANO1, | |
| | CYP27B1, SNX5, LYAR, LDLRAD3, GPD1L, ELAVL2, | |
| | MAL2, GAS2, PBX1, CD302, DEPDC4, ITPR1, SLIRP, | |
| | SCN2B, NUP107, KRT18P32, RBM47, KRT18P28, | |
| | SLC39A8, KRT18P20, CENPL, MESP1, SLC26A2, | |
| | POLR1B, PTPN4, HS6ST2, RAE1, CFD, | |
| | COBLL1, CHAC2, EAF2, PLA2G4A, CD47, NOXI | |
| TGFa_30 min | EREG, VEGFA, HBEGF, IL1B, IL6, IL1A, CXCL3, CXCL2, | Cheng et al. |
| | CXCL8, EDN1, ZYX, DUSP1, PTGS2, DUSP6, ARHGEF2, | 2008 [97] |
| | PHLDA1, | |
| | JUN, EGR1, FOS, CITED2, DDIT3, DUSP4, PPP1R15A, | |
| | TNFAIP3, NOP14-AS1 | |
| TGFa_60 min | ARTN, VEGFA, HBEGF, IL1B, LIF, IL11, ILIA, IL36G, | Cheng et al. |
| | SERPINE1, FSTL3, SLPI, PI3, THBD, CLDN4, EPHA2, | 2008 [97] |
| | PLAUR, JMJD6, YRDC, RHCG, ULBP2, SPRY4, GNE, | |
| | PLEKHG3, PTPRE, TUBB2A, TUBB2B, PKP1, MAT2A, | |
| | PTGS2, HS3ST1, FUT1, PLK3, FBXL18, SLC20A1, ALAS1, | |
| | ISG20L2, SPRR2B, NLRP1, GRPELI, CAPN3, SPRR1A, | |
| | IVL, PHLDA1, FOSLI, EGR3, ID1, FOS, ZFP36L2, | |
| | CDKN1A, IER2, DDIT4, COA1, KDM6B, INAVA, | |
| | LGALSL, LRRC8E, AKIP1, NUAK2, C1orf56, PDXDC1 | |
| TGFa_120 min | EREG, IL1B, IL36G, CXCL2, CXCL8, MMP1, MMP10, | Cheng et al. |
| | PTP4A1, MAT2A, CYP27B1, SCG5, PHLDA1, ARL4C | 2008 [97] |
| TNF_A | CNFN, TREX2, SPRR2B, SPRR2D, SULT2B1 | Li et al. 2014 |
| | | [107] |
| TNFa_B | MMP9, C1QTNF1, IL36G, TNFAIP3, CXCL8, IRAK2, | Chiricozzi et |
| | CXCL10, TYMP, IL1B, SOD2, NFKBIA, S100A9, HBEGF, | al. 2011 [102] |
| | PLAU, HLA-B, | |
| | MMP10, PI3, BIRC3, KREMEN2, WNT4, ISG15, PRDM1, | |
| | TNFSF10, IL1RN, TNIP1, SERPINB2, IRF1, S100A8, | |
| | DRAM1, LYN, NFKBIZ, CCL27, C1orf74, MUC1, ELF3, | |
| | IFIT3, RELB, RHCG, DUSP1, C1R, CSF2, TNFRSF6B, | |
| | CXCL16, PIK3IP1 | |
| TNFa_C | BIRC3, ZC3H12C, CXCL10, NINJ1, LACC1, PPPIR3C, | Yao et al. |
| | RELB, CPNE8, COL12A1, BID, PLAU, STEAP4, DDX58, | 2008 [100] |
| | DRAM1, BDKRB1, GCH1, SDC4, IFIH1, TNFAIP3, TAP1, | |
| | BCL3, SRCAP, CCL20, CYLD, SOD2, PSMB8, | |
| | TNFRSF10B, FZD8, FAS, | |
| | PSTPIP2, TSLP, MMP28 | |
| TNFa_early | CEP135, EFNA1, MARCKSL1, ICAM1, NINJ1, CFLAR, | Banno et al. |
| | CFB, IVL, S100A8, S100A9, SPRR2A, TGM1, NEFM, | 2004 [95] |
| | TPST1, H1-10, | |
| | IFIT3, KRT34, PDPN, SCO2, MMP10, MMP9, RAB32, | |
| | TNFAIP2, CCL20, CCL27, CCL5, CXCL10, CXCL11, | |
| | CXCL1, CXCL2, CXCL3, CXCL8, BMP2, IL1RN, LTB, | |
| | IL32, TNF, TNFSF10, VEGFC, IRF1, NFKBIA, SOX4, | |
| | TNFAIP3, TAP1, ATP12A | |
| TNFa_late | KYNU, TNC, TRAF1, PSME2, PSMB10, PSMB9, BTG3, | Banno et al. |
| | C1R, C1S, NEDD9, CSPG4, COL16A1, GALT, H2AC18, | 2004 [95] |
| | H2AC20, HLA- F, HLA-A, MR1, SAA1, OAS1, OASL, | |
| | CD47, ITGB6, CDH3, KRT2, LYN, ACOXI, CD58, | |
| | PDZK1IP1, GPD2, SOD2, PML, DUSP1, ALOX12B, GGH, | |
| | MMP13, WFDC2, SPINK5, TLR1, NCK1, BST2, TYMP, | |
| | LSS, HIVEP1, LITAF, SMAD1, ZFYVE9, MEOX1, | |
| | RELB, SLC39A8, DLC1 | |
| TNFa_IFNg | IL32, CCL8, CXCL3, CXCL8, CCL20, CXCL5, CCL17, | Fujita et al. |
| | CCL2, CXCL14, CCL5, CCL22, IFNL1, IFNB1, IFNL2, | 2011 [103] |
| | IFNL3, IL6, IL1B, | |
| | CCL4, TNF, LTB, IL36G, IL33, CSF2, CXCL1, IL24, CCL7, | |
| | IL11, IFNE, CXCL16, IL16 | |

TABLE 5A
Class balance strategy used for machine learning classification.

| | | | Class | | |
|---|---|---|---|---|---|
| | Original | ML | balance | | Greedy |
| Comparison | Samples | Samples | stratagy | Class | Elimination |

Lesional Skin

| | | | | | | | |
|---|---|---|---|---|---|---|---|
| (A) | DLE vs | 90 DLE vs | 90 DLE vs | None | Class 0 | CTL | IL12, |
| | CTL | 164 CTL | 164 CTL | | Class 1 | DLE | Monocyte/ |
| | | | | | | | Myeloid Cell |
| (B) | AD vs | 132 AD vs | 132 AD vs | None | Class 0 | CTL | Monocyte, IL12, |
| | CTL | 164 CTL | 164 CTL | | Class 1 | AD | Peroxisome |
| (C) | SSc vs | 97 SSc vs | 97 SSc vs | Random | Class 0 | CTL | Monocyte, IL12, |
| | CTL | 164 CTL | 97CTL | Under- | Class 1 | SSc | Complement |
| | | | | sampling | | | Proteins, |
| | | | | | | | Peroxisomes, |
| | | | | | | | Monocyte/ |
| | | | | | | | Myeloid Cell |
| (D) | PSO vs | 183 PSO vs | 183 PSO vs | None | Class 0 | CTL | IL12, Monocyte |
| | CTL | 164 CTL | 164 CTL | | Class 1 | PSO | |
| (E) | DLE vs | 90 DLE vs | 90 DLE vs | Random | Class 0 | SUB | None |
| | SUB | 54 SUB | 90 SUB | Oversample | Class 1 | DLE | |
| (F) | DLE vs | 90 DLE vs | 90 DLE vs | Excluded | Class 0 | PSO | None |
| | PSO | 183 PSO | 110 PSO | GSE117468 | Class 1 | DLE | |
| (G) | DLE vs | 90 DLE vs | 90 DLE vs | None | Class 0 | AD | Monocyte/Myeloid |
| | AD | 132 AD | 132 AD | | Class 1 | DLE | Cell, TNF |
| (H) | DLE vs | 90 DLE vs | 90 DLE vs | None | Class 0 | SSc | FABO, TNF |
| | SSc | 97 SSc | 97 SSc | | Class 1 | DLE | |

Nonlesional Skin

| | | | | | | | |
|---|---|---|---|---|---|---|---|
| (I) | DLE vs | 21 DLE vs | 98 DLE vs | SMOTE | Class 0 | CTL | None |
| | CTL | 164 CTL | 122 CTL | over and | Class 1 | DLE | |
| | | | | undersample | | | |
| (J) | PSO vs | 163 PSO vs | 163 PSO vs | Excluded | Class 0 | CTL | Monocyte |
| | CTL | 164 CTL | 164 CTL | PSO from | Class 1 | PSO | |
| | | | | GSE52471 | | | |
| | | | | and | | | |
| | | | | GSE109248 | | | |
| (K) | AD vs | 132 AD vs | 164 AD vs | Random | Class 0 | CTL | Monocyte |
| | CTL | 164 CTL | 164 CTL | Oversample | Class 1 | AD | |
| (L) | DLE vs | 21 DLE vs | 97 DLE vs | SMOTE | Class 0 | PSO | Monocyte, |
| | PSO | 163 PSO | 121 PSO | over and | Class 1 | DLE | FABO, IL12 |
| | | | | undersample | | | |
| (M) | DLE vs | 21 DLE vs | 79 DLE vs | SMOTE | Class 0 | AD | Monocyte, T Cell, |
| | AD | 132 AD | 98 AD | over and | Class 1 | DLE | FABO, IL12 |
| | | | | undersample | | | |
| (N) | PSO vs | 163 PSO vs | 163 PSO vs | None | Class 0 | AD | Monocyte/Myeloid |
| | AD | 132 AD | 132 AD | | Class 1 | PSO | Cell, Complement |
| | | | | | | | Proteins, |
| | | | | | | | Peroxisome, IL12 |

TABLE 5B
Number of samples pooled from each skin dataset
to create input for machine learning.

| | | | Number of |
|---|---|---|---|
| | Samples | Datasets | samples |
| Lesional | 90 DLE | GSE52471 | 7 |
| samples | | GSE72535 | 9 |
| | | GSE81071_A | 26 |
| | | GSE81071_B | 21 |
| | | GSE109248 | 6 |
| | | GSE100093 | 15 |
| | | GSE120809 | 6 |
| | 132 AD | GSE130588 | 19 |
| | | GSE137430 | 38 |
| | | GSE157194 | 54 |
| | | GSE121212 | 21 |
| | 183 PSO | GSE52471 | 3 |
| | | GSE109248 | 17 |
| | | GSE117239 | 63 |
| | | GSE117468 | 73 |
| | | GSE121212 | 27 |
| | 97 SSc | GSE58095 | 39 |
| | | GSE130955 | 58 |
| Non Lesional | 21 DLE | GSE100093 | 15 |
| samples | | GSE120809 | 6 |
| | 132 AD | GSE130588 | 19 |
| | | GSE137430 | 38 |
| | | GSE157194 | 54 |
| | | GSE121212 | 21 |
| | 163 PSO | GSE117239 | 63 |
| | | GSE117468 | 73 |
| | | GSE121212 | 27 |
| Healthy | 164 CTL | GSE52471 | 3 |
| control | | GSE72535 | 8 |
| samples | | GSE81071_A | 7 |
| | | GSE81071_B | 6 |
| | | GSE109248 | 13 |
| | | GSE130588 | 20 |
| | | GSE121212 | 38 |
| | | GSE58065 | 36 |
| | | GSE130955 | 33 |

TABLE 6
Comparison between Z-score GSVA and mean of Z-score.

| | | P value Welch's T test | | | | | |
|---|---|---|---|---|---|---|---|
| | | NL DLE and CTL | | NL PSO and CTL | | NL ATD and CTL | |
| | # of | Z-score | GSVA | Z-score | GSVA | Z-score | GSVA |
| Gene Signature | genes | mean | score | mean | score | mean | score |
| AA Metabolism | 12 | ** | * | * | ** | * | ns |
| Anti inflammation | 3 | ns | ns | ns | ns | ns | * |
| Apoptosis | 13 | * | ns | **** | **** | **** | **** |
| B Cell | 4 | ** | *** | ns | ns | ns | ns |
| Cell Cycle | 18 | ns | ns | **** | ns | **** | ns |
| Complement Proteins | 7 | ** | **** | ns | * | **** | **** |
| Endothelial Cell | 11 | **** | **** | **** | **** | **** | **** |
| FAAO | 4 | **** | **** | **** | **** | ** | ** |
| FABO | 44 | * | ** | **** | *** | ** | ns |
| Fibroblast | 63 | **** | **** | **** | *** | **** | **** |
| Glycolysis | 20 | * | ** | **** | **** | ns | * |
| Granulocyte | 2 | **** | **** | **** | * | **** | **** |
| IFN | 21 | ns | ns | **** | **** | **** | **** |
| IL12 | 36 | **** | **** | **** | **** | ns | * |
| IL17 Complex | 4 | * | **** | **** | **** | * | ** |
| Inflammasome | 12 | **** | **** | ns | **** | **** | **** |
| Keratinocyte | 57 | ns | ns | *** | * | ns | ns |
| Langerhans Cell | 4 | **** | **** | **** | **** | ns | ns |
| Metanocyle | 8 | **** | ** | **** | **** | **** | * |
| Monocyte | 8 | ns | ns | ns | ns | ns | ns |
| Monocyte/Myeloid Cell | 40 | **** | **** | **** | **** | ns | ns |
| NeutrophIl | 5 | **** | ** | **** | **** | **** | **** |
| NK Cell | 2 | ns | * | **** | **** | **** | **** |
| OXPHOS | 74 | ns | ns | **** | **** | ns | ns |
| pDC | 3 | ns | * | **** | **** | ns | ns |
| Pentose Phosphate | 9 | ** | ** | **** | **** | **** | **** |
| Peroxisome | 25 | **** | **** | **** | ** | ns | ns |
| Plasma Cell | 10 | **** | *** | **** | ** | **** | **** |
| Platelet | 3 | **** | **** | **** | **** | **** | **** |
| Proteasome | 41 | ns | ns | **** | **** | * | ns |
| ROS Production | 2 | ns | * | ns | **** | **** | **** |
| Skin-specific DC | 3 | ns | *** | **** | * | **** | **** |
| T Cell | 9 | ns | ns | **** | **** | ** | ns |
| T Cell IL12 Signature | 3 | **** | **** | **** | **** | ns | ns |
| T Cell IL23 Signature | 7 | * | ns | ns | ns | ** | **** |
| TCA Cycle | 32 | ns | ns | **** | **** | *** | **** |
| Th17 | 2 | ** | ns | ns | *** | *** | **** |
| TNF | 86 | **** | **** | ns | * | **** | **** |
| TGFB Fibroblast | 229 | ns | ns | **** | **** | ns | ns |
| Unfolded Protein | 40 | ns | ns | **** | **** | ns | * |
| | | Hedges' g Effect Size | | | | | |
| | | NL DLE and CTL | | NL PSO and CTL | | NL ATD and CTL | |
| | # of | Z-score | GSVA | Z-score | GSVA | Z-score | GSVA |
| Gene Signature | genes | mean | score | mean | score | mean | score |
| AA Metabolism | 12 | 0.85 | 0.44 | 0.25 | 0.35 | −0.23 | 0.17 |
| Anti inflammation | 3 | −0.12 | −0.12 | 0.00 | 0.16 | 0.07 | 0.25 |
| Apoptosis | 13 | 0.24 | 0.15 | −0.89 | −1.08 | −1.08 | −1.23 |
| B Cell | 4 | 0.72 | 0.95 | 0.08 | 0.10 | −0.17 | −0.08 |
| Cell Cycle | 18 | −0.01 | −0.19 | 0.88 | −0.01 | 0.46 | 0.14 |
| Complement Proteins | 7 | 0.38 | 0.45 | −0.10 | 0.24 | −1.87 | −1.85 |
| Endothelial Cell | 11 | −1.79 | −1.71 | −1.16 | −0.61 | −0.84 | −0.93 |
| FAAO | 4 | 1.46 | 1.47 | 1.72 | 1.67 | −0.35 | −0.39 |
| FABO | 44 | 1.09 | 0.92 | −0.53 | −0.36 | −0.36 | −0.13 |
| Fibroblast | 63 | 1.64 | 1.00 | 1.33 | 0.43 | 1.73 | 1.35 |
| Glycolysis | 20 | −0.86 | −0.82 | −2.07 | −1.83 | 0.08 | 0.24 |
| Granulocyte | 2 | −1.26 | −1.44 | −0.67 | −0.23 | −0.60 | −0.74 |
| IFN | 21 | 0.39 | −0.04 | −0.63 | −0.63 | −1.53 | −0.69 |
| IL12 | 36 | −1.54 | −2.16 | −0.99 | −0.63 | 0.13 | 0.26 |
| IL17 Complex | 4 | 0.46 | 0.87 | 0.84 | 0.89 | 0.26 | 0.36 |
| Inflammasome | 12 | −0.78 | −1.20 | 0.02 | −0.17 | −0.66 | −0.83 |
| Keratinocyte | 57 | 0.74 | 0.62 | −0.40 | −0.26 | −0.18 | −0.09 |
| Langerhans Cell | 4 | −1.44 | −1.37 | −1.19 | −1.31 | −0.16 | −0.21 |
| Metanocyle | 8 | 0.91 | 0.62 | 0.68 | 0.62 | 0.75 | 0.28 |
| Monocyte | 8 | −0.19 | −0.21 | 0.10 | 0.14 | 0.08 | −0.03 |
| Monocyte/Myeloid Cell | 40 | 0.67 | 0.71 | 1.24 | 1.15 | −0.15 | −0.12 |
| NeutrophIl | 5 | 0.62 | −0.40 | 0.68 | 0.79 | 0.68 | 0.87 |
| NK Cell | 2 | 0.18 | 0.56 | 1.16 | 1.13 | 1.69 | 1.79 |
| OXPHOS | 74 | 0.49 | 0.12 | −0.94 | −1.15 | 0.02 | 0.15 |
| pDC | 3 | −0.74 | −0.87 | −1.89 | −1.79 | −0.05 | −0.01 |
| Pentose Phosphate | 9 | −0.91 | −1.14 | −1.17 | −1.18 | 1.48 | 0.95 |
| Peroxisome | 25 | 1.36 | 1.12 | 1.31 | 0.31 | 0.13 | −0.01 |
| Plasma Cell | 10 | 0.79 | 0.58 | 0.62 | 0.36 | 0.72 | 0.49 |
| Platelet | 3 | −1.18 | −0.89 | −0.86 | −0.44 | −0.61 | −0.66 |
| Proteasome | 41 | −0.17 | −0.32 | −1.13 | −0.98 | 0.29 | 0.15 |
| ROS Production | 2 | 0.47 | 0.75 | 0.16 | 0.77 | 1.17 | 1.64 |
| Skin-specific DC | 3 | 0.17 | −0.92 | 1.00 | 0.24 | 2.03 | 1.79 |
| T Cell | 9 | −0.20 | −0.10 | 0.78 | 0.66 | −0.35 | −0.08 |
| T Cell IL12 Signature | 3 | −0.89 | −0.78 | −1.00 | −0.71 | 0.08 | 0.19 |
| T Cell IL23 Signature | 7 | −0.46 | 0.31 | −0.11 | −0.16 | −0.32 | 0.46 |
| TCA Cycle | 32 | 0.34 | 0.73 | −1.32 | −0.72 | 0.39 | 0.75 |
| Th17 | 2 | −0.34 | −0.09 | −0.18 | 0.41 | 0.39 | 0.74 |
| TNF | 86 | −1.02 | −1.09 | −0.08 | −0.26 | 0.72 | 0.55 |
| TGFB Fibroblast | 229 | 0.24 | 0.10 | −0.84 | −0.63 | 0.03 | 0.22 |
| Unfolded Protein | 40 | −0.21 | −0.24 | −0.90 | −0.65 | −0.08 | 0.24 |

TABLE 7
Classification metrics to properly separate DLE, PSO, AD or SSc and control samples
using all 48 (top) or the top 15 (bottom) cellular and pathway gene signatures.

| Random | | Skin | | | Cohen | | f-1 | |
|---|---|---|---|---|---|---|---|---|
| Forest | Features | Type | Sensitivity | Specificity | Kappa | Precision | score | Accuracy |
| DLE vs | All features | LS | 0.93 | 0.98 | 0.91 | 0.96 | 0.94 | 0.96 |
| CTL | top 15 features | LS | 0.96 | 0.96 | 0.91 | 0.93 | 0.94 | 0.96 |
| PSO vs | All features | LS | 0.95 | 0.88 | 0.82 | 0.90 | 0.92 | 0.91 |
| CTL | top 15 features | LS | 0.91 | 0.88 | 0.78 | 0.89 | 0.89 | 0.90 |
| AD vs | All features | LS | 0.84 | 0.98 | 0.81 | 0.97 | 0.90 | 0.91 |
| CTL | top 15 features | LS | 0.84 | 0.98 | 0.81 | 0.97 | 0.90 | 0.91 |
| SSc vs | All features | LS | 0.87 | 0.89 | 0.76 | 0.90 | 0.89 | 0.88 |
| CTL | top 15 features | LS | 0.84 | 0.86 | 0.69 | 0.87 | 0.85 | 0.85 |

TABLE 8
Classification metrics including sensitivity, specificity, Cohen
Kappa score, precision, f-1 score and accuracy to properly
separate lesional disease samples (DLE, PSO, AD or SSc)
from healthy control sampleswith each ML classifier
Disease vs. Control Lesional Results

| | | | | | Cohen | | f-1 | |
|---|---|---|---|---|---|---|---|---|
| Comparison | | Classifier | Sensitivity | Specificity | Kappa | Precision | score | Accuracy |
| CTL | DLE | Logistic R | 0.89 | 0.94 | 0.83 | 0.89 | 0.89 | 0.92 |
| | PSO | | 0.96 | 0.90 | 0.87 | 0.91 | 0.93 | 0.93 |
| | AD | | 0.84 | 0.89 | 0.73 | 0.88 | 0.85 | 0.87 |
| | SSc | | 0.90 | 0.89 | 0.80 | 0.90 | 0.90 | 0.90 |
| | DLE | Random F | 0.93 | 0.98 | 0.92 | 0.96 | 0.94 | 0.96 |
| | PSO | | 0.95 | 0.88 | 0.82 | 0.90 | 0.92 | 0.91 |
| | AD | | 0.84 | 0.98 | 0.82 | 0.97 | 0.90 | 0.91 |
| | SSc | | 0.87 | 0.89 | 0.76 | 0.90 | 0.88 | 0.88 |
| | DLE | SVM | 0.89 | 0.98 | 0.88 | 0.96 | 0.92 | 0.95 |
| | PSO | | 0.93 | 0.92 | 0.84 | 0.93 | 0.93 | 0.92 |
| | AD | | 0.86 | 0.99 | 0.86 | 0.99 | 0.92 | 0.93 |
| | SSc | | 0.90 | 0.93 | 0.83 | 0.93 | 0.91 | 0.92 |
| | DLE | Naïve | 0.96 | 0.94 | 0.88 | 0.90 | 0.92 | 0.95 |
| | PSO | Bayes | 0.89 | 0.94 | 0.82 | 0.94 | 0.91 | 0.91 |
| | AD | | 0.86 | 0.91 | 0.77 | 0.90 | 0.88 | 0.89 |
| | SSc | | 0.84 | 0.86 | 0.69 | 0.87 | 0.85 | 0.85 |
| | DLE | KNN | 0.82 | 0.96 | 0.79 | 0.92 | 0.86 | 0.91 |
| | PSO | | 0.89 | 0.96 | 0.84 | 0.96 | 0.92 | 0.92 |
| | AD | | 0.86 | 0.99 | 0.86 | 0.99 | 0.92 | 0.93 |
| | SSc | | 0.81 | 0.96 | 0.76 | 0.96 | 0.87 | 0.88 |
| | DLE | GB | 0.86 | 0.96 | 0.83 | 0.92 | 0.89 | 0.92 |
| | PSO | | 0.95 | 0.88 | 0.83 | 0.90 | 0.92 | 0.91 |
| | AD | | 0.86 | 0.98 | 0.84 | 0.97 | 0.91 | 0.92 |
| | SSc | | 0.84 | 0.89 | 0.73 | 0.90 | 0.87 | 0.86 |

TABLE 9
Classification metrics to properly separate lesional DLE samples and
lesional PSO or lesional AD or lesional SSc samples using all 48 (top)
or the top 15 (bottom) cellular and pathway gene signatures.

| Random | | Skin | | | Cohen | | f-1 | |
|---|---|---|---|---|---|---|---|---|
| Forest | Features | Type | Sensitivity | Specificity | Kappa | Precision | score | Accuracy |
| DLE vs | All features | LS | 0.63 | 0.88 | 0.51 | 0.81 | 0.70 | 0.77 |
| PSO | top 15 features | LS | 0.70 | 0.85 | 0.55 | 0.79 | 0.74 | 0.78 |
| DLE vs | All features | LS | 0.42 | 0.90 | 0.35 | 0.73 | 0.53 | 0.72 |
| AD | top 15 features | LS | 0.58 | 0.93 | 0.53 | 0.83 | 0.68 | 0.79 |
| DLE vs | All features | LS | 0.62 | 0.84 | 0.46 | 0.76 | 0.68 | 0.74 |
| SSc | top 15 features | LS | 0.62 | 0.81 | 0.43 | 0.73 | 0.67 | 0.72 |

TABLE 10
Classification metrics including sensitivity, specificity, Cohen
Kappa score, precision, f-1 score and accuracy to properly
separate lesional DLE samples from lesional PSO, AD,
and SSc samples with each ML classifier.
DLE vs. Disease Lesional Results

| | | | | | Cohen | | f-1 | |
|---|---|---|---|---|---|---|---|---|
| Comparison | | Classifier | Sensitivity | Specificity | Kappa | Precision | score | Accuracy |
| DLE | PSO | Logistic R | 0.78 | 0.91 | 0.69 | 0.88 | 0.83 | 0.85 |
| | AD | | 0.50 | 0.78 | 0.29 | 0.59 | 0.54 | 0.67 |
| | SSc | | 0.65 | 0.77 | 0.43 | 0.71 | 0.68 | 0.72 |
| | PSO | Random F | 0.63 | 0.88 | 0.52 | 0.81 | 0.71 | 0.77 |
| | AD | | 0.42 | 0.90 | 0.35 | 0.73 | 0.53 | 0.72 |
| | SSc | | 0.62 | 0.84 | 0.46 | 0.76 | 0.68 | 0.74 |
| | PSO | SVM | 0.81 | 0.97 | 0.80 | 0.96 | 0.88 | 0.90 |
| | AD | | 0.65 | 0.93 | 0.61 | 0.85 | 0.74 | 0.82 |
| | SSc | | 0.69 | 0.90 | 0.60 | 0.86 | 0.77 | 0.81 |
| | PSO | Naïve | 0.67 | 0.85 | 0.52 | 0.78 | 0.72 | 0.77 |
| | AD | Bayes | 0.65 | 0.83 | 0.49 | 0.71 | 0.68 | 0.76 |
| | SSc | | 0.62 | 0.81 | 0.43 | 0.73 | 0.67 | 0.72 |
| | PSO | KNN | 0.63 | 0.94 | 0.58 | 0.89 | 0.74 | 0.80 |
| | AD | | 0.69 | 0.93 | 0.64 | 0.86 | 0.77 | 0.84 |
| | SSc | | 0.73 | 0.81 | 0.54 | 0.76 | 0.74 | 0.77 |
| | PSO | GB | 0.70 | 0.85 | 0.56 | 0.79 | 0.74 | 0.78 |
| | AD | | 0.69 | 0.85 | 0.55 | 0.75 | 0.72 | 0.79 |
| | SSc | | 0.69 | 0.81 | 0.50 | 0.75 | 0.72 | 0.75 |

TABLE 11
Classification metrics to properly separate nonlesional DLE and control samples,
nonlesional PSO and control samples, as well as nonlesional AD and control samples
using all 48 (top) or the top 15 (bottom) cellular and pathway gene signatures.

| Random | | Skin | | | Cohen | | f-1 | |
|---|---|---|---|---|---|---|---|---|
| Forest | Features | Type | Sensitivity | Specificity | Kappa | Precision | score | Accuracy |
| DLE vs | All features | LS | 0.94 | 0.97 | 0.90 | 0.97 | 0.95 | 0.95 |
| CTL | top 15 features | LS | 0.94 | 1.00 | 0.93 | 1.00 | 0.96 | 0.97 |
| PSO vs | All features | LS | 0.75 | 0.90 | 0.63 | 0.91 | 0.82 | 0.82 |
| CTL | top 15 features | LS | 0.63 | 0.79 | 0.40 | 0.80 | 0.70 | 0.70 |
| AD vs | All features | LS | 0.75 | 0.94 | 0.67 | 0.93 | 0.83 | 0.84 |
| CTL | top 15 features | LS | 0.67 | 0.81 | 0.47 | 0.80 | 0.72 | 0.74 |

TABLE 12
Classification metrics including sensitivity, specificity, Cohen
Kappa score, precision, f-1 score and accuracy to properly
separate nonlesional disease samples (DLE, PSO or AD) from
healthy control samples with each ML classifier.
Disease vs. Control Nonlesional Results

| | | | | | Cohen | | f-1 | |
|---|---|---|---|---|---|---|---|---|
| Comparison | | Classifier | Sensitivity | Specificity | Kappa | Precision | score | Accuracy |
| Control | Lupus | Logistic R | 0.94 | 0.94 | 0.88 | 0.94 | 0.94 | 0.94 |
| | Psoriasis | | 0.72 | 0.79 | 0.49 | 0.82 | 0.77 | 0.75 |
| | Atopic D | | 0.63 | 0.81 | 0.44 | 0.79 | 0.70 | 0.72 |
| | Lupus | Random F | 0.94 | 0.97 | 0.91 | 0.97 | 0.95 | 0.95 |
| | Psoriasis | | 0.75 | 0.90 | 0.64 | 0.91 | 0.82 | 0.82 |
| | Atopic D | | 0.75 | 0.94 | 0.68 | 0.93 | 0.83 | 0.84 |
| | Lupus | SVM | 0.94 | 0.94 | 0.88 | 0.94 | 0.94 | 0.94 |
| | Psoriasis | | 0.75 | 0.90 | 0.64 | 0.91 | 0.82 | 0.82 |
| | Atopic D | | 0.83 | 0.85 | 0.68 | 0.86 | 0.84 | 0.84 |
| | Lupus | Naïve | 0.91 | 0.94 | 0.85 | 0.94 | 0.92 | 0.92 |
| | Psoriasis | Bayes | 0.77 | 0.76 | 0.53 | 0.81 | 0.79 | 0.77 |
| | Atopic D | | 0.58 | 0.83 | 0.40 | 0.79 | 0.67 | 0.70 |
| | Lupus | KNN | 0.99 | 0.61 | 0.63 | 0.74 | 0.85 | 0.82 |
| | Psoriasis | | 0.77 | 0.83 | 0.59 | 0.86 | 0.81 | 0.80 |
| | Atopic D | | 0.56 | 0.91 | 0.46 | 0.88 | 0.68 | 0.73 |
| | Lupus | GB | 0.94 | 0.90 | 0.85 | 0.92 | 0.93 | 0.92 |
| | Psoriasis | | 0.68 | 0.79 | 0.46 | 0.81 | 0.74 | 0.73 |
| | Atopic D | | 0.79 | 0.85 | 0.64 | 0.85 | 0.82 | 0.82 |

TABLE 13
Classification metrics to properly separate DLE samples and PSO or AD samples
using all 48 (top) or the top 15 (bottom) cellular and pathway gene signatures.

| Random | | Skin | | | Cohen | | f-1 | |
|---|---|---|---|---|---|---|---|---|
| Forest | Features | Type | Sensitivity | Specificity | Kappa | Precision | score | Accuracy |
| DLE vs | All features | NL | 0.99 | 0.94 | 0.93 | 0.94 | 0.96 | 0.97 |
| PSO | top 15 features | NL | 0.99 | 0.97 | 0.96 | 0.97 | 0.98 | 0.98 |
| DLE VS | All features | NL | 0.88 | 0.97 | 0.84 | 0.95 | 0.91 | 0.93 |
| AD | top 15 features | NL | 0.88 | 0.93 | 0.81 | 0.91 | 0.89 | 0.91 |

TABLE 14
Classification metrics including sensitivity, specificity, Cohen
Kappa score, precision, f-1 score and accuracy toproperly
separate nonlesional DLE samples from nonlesional PSO
and nonlesional AD samples with each ML classifier.
DLE vs. Disease Nonlesional Results

| | | | | | Cohen | | f-1 | |
|---|---|---|---|---|---|---|---|---|
| Comparison | | Classifier | Sensitivity | Specificity | Kappa | Precision | score | Accuracy |
| DLE | PSO | Logistic R | 0.97 | 0.94 | 0.91 | 0.94 | 0.95 | 0.95 |
| | AD | | 0.92 | 0.97 | 0.89 | 0.96 | 0.94 | 0.94 |
| | PSO | Random F | 0.99 | 0.94 | 0.94 | 0.94 | 0.97 | 0.97 |
| | AD | | 0.88 | 0.97 | 0.85 | 0.95 | 0.91 | 0.93 |
| | PSO | SVM | 0.99 | 0.97 | 0.97 | 0.97 | 0.98 | 0.98 |
| | AD | | 0.96 | 0.99 | 0.96 | 0.99 | 0.98 | 0.98 |
| | PSO | Naïve | 0.90 | 0.97 | 0.88 | 0.97 | 0.93 | 0.94 |
| | AD | Bayes | 0.92 | 0.93 | 0.85 | 0.92 | 0.92 | 0.93 |
| | PSO | KNN | 0.99 | 0.83 | 0.82 | 0.84 | 0.91 | 0.91 |
| | AD | | 0.96 | 0.80 | 0.74 | 0.79 | 0.87 | 0.87 |
| | PSO | GB | 0.94 | 0.91 | 0.85 | 0.91 | 0.92 | 0.92 |
| | AD | | 0.88 | 0.93 | 0.81 | 0.91 | 0.89 | 0.91 |

TABLE 15
Classification metrics including sensitivity, specificity, Cohen Kappa
score, precision, f-1 score and accuracy to properly separate nonlesional
PSO samples from nonlesional AD samples with each ML classifier.
NL PSO vs. NL AD

| | Sensi- | Speci- | Cohen | Preci- | f-1 | Accu- |
|---|---|---|---|---|---|---|
| Classifier | tivity | ficity | Kappa | sion | score | racy |
| Logistic R | 0.78 | 0.48 | 0.26 | 0.6 | 0.68 | 0.63 |
| Random F | 0.8 | 0.64 | 0.44 | 0.69 | 0.74 | 0.72 |
| SVM | 0.78 | 0.57 | 0.35 | 0.65 | 0.71 | 0.67 |
| Naïve | 0.78 | 0.57 | 0.35 | 0.65 | 0.71 | 0.67 |
| Bayes | | | | | | |
| KNN | 0.87 | 0.48 | 0.35 | 0.63 | 0.73 | 0.67 |
| GB | 0.84 | 0.61 | 0.46 | 0.69 | 0.76 | 0.73 |

TABLE 16
Classification metrics including sensitivity, specificity,
Cohen Kappa score, precision, f-1 score and accuracy
to properly separate DLE and SCLE.
DLE vs. SCLE Results

| | Sensi- | Speci- | Cohen | Preci- | f-1 | Accu- |
|---|---|---|---|---|---|---|
| Classifier | tivity | ficity | Kappa | sion | score | racy |
| Logistic R | 0.72 | 0.79 | 0.51 | 0.75 | 0.73 | 0.76 |
| Random F | 0.72 | 0.90 | 0.62 | 0.86 | 0.78 | 0.81 |
| SVM | 0.64 | 0.93 | 0.58 | 0.89 | 0.74 | 0.80 |
| GB | 0.68 | 0.83 | 0.51 | 0.77 | 0.72 | 0.76 |

### Abbreviations

Systemic lupus erythematosus (SLE)
Cutaneous lupus erythematosus (CLE)
Acute cutaneous lupus erythematosus (ACLE)
Subacute cutaneous lupus erythematosus (SCLE)
Chronic cutaneous lupus erythematosus (CCLE)
Chronic discoid lupus erythematosus (CDLE) or (DLE)
Atopic dermatitis (AD) or atopic eczema
Psoriasis (PSO)
Systemic sclerosis (SSc) or scleroderma
Gene set variation analysis (GSVA)
Machine learning (ML)
Random forest (RF)
Area under the receiver operating characteristic curve (AUROC curve)
Area under the precision-recall curve (AUPR curve)
Synthetic minority oversampling technique (SMOTE)
Low-density granulocyte (LDG)
Skin-specific dendritic cell (Skin-specific DC)
Plasmacytoid dendritic cell (pDC)
Natural killer (NK) cell
Germinal center (GC) B cell
Interferon (IFN)
Interferon gene signature (IGS)
Interleukin (IL)
Transforming growth factor beta (TGFO)
Tumor necrosis factor (TNF)
Reactive Oxygen Species production (ROS production)
Tricarboxylic acid cycle (TCA)
Oxidative phosphorylation (OXPHOS)
Fatty acid alpha oxidation (FAAO)
Fatty acid beta oxidation (FABO)
Amino acid metabolism (AA metabolism)
T helper (Th)
T follicular helper (Tfh)

### References (Each Incorporated Herein by Reference in its Entirety)

1. B. Tebbe, C. Orfanos, Epidemiology and socioeconomic impact of skin disease in lupus erythematosus, Lupus 6, 96-104 (1997).
2. M. P. Maz, J. Michelle Kahlenberg, Cutaneous and systemic connections in lupus, Curr. Opin. Rheumatol. 32 (2020), doi:10.1097/BOR.0000000000000739.
3. L. Uva, D. Miguel, C. Pinheiro, J. P. Freitas, M. Marques Gomes, P. Filipe, Cutaneous manifestations of systemic lupus erythematosus, Autoimmune Dis. 1 (2012), doi:10.1155/2012/834291.
4. J. Wenzel, Cutaneous lupus erythematosus: new insights into pathogenesis and therapeutic strategies, Nat. Rev. Rheumatol. 15, 519-532 (2019).
5. S. Ribero, S. Sciascia, L. Borradori, D. Lipsker, The Cutaneous Spectrum of Lupus Erythematosus, Clin. Rev. Allergy Immunol. 53, 291-305 (2017).
6. P. Vashisht, K. Borghoff, J. R. O'Dell, M. Hearth-Holmes, Belimumab for the treatment of recalcitrant cutaneous lupus, Lupus 26, 857-864 (2017).
7. E. F. Morand, R. Furie, Y. Tanaka, I. N. Bruce, A. D. Askanase, C. Richez, S.-C. Bae, P. Z. Brohawn, L. Pineda, A. Berglind, R. Tummala, Trial of Anifrolumab in Active Systemic Lupus Erythematosus, N. Engl. J. Med. 382, 211-221 (2020).
8. A. Menter, B. E. Strober, D. H. Kaplan, D. Kivelevitch, E. F. Prater, B. Stoff, A. W. Armstrong, C. Connor, K. M. Cordoro, D. M. R. Davis, B. E. Elewski, J. M. Gelfand, K. B. Gordon, A. B. Gottlieb, A. Kavanaugh, M. Kiselica, N. J. Korman, D. Kroshinsky, M. Lebwohl, C. L. Leonardi, J. Lichten, H. W. Lim, N. N. Mehta, A. S. Paller, S. L. Parra, A. L. Pathy, R. N. Rupani, M. Siegel, E. B. Wong, J. J. Wu, V. Hariharan, C. A. Elmets, Joint AAD-NPF guidelines of care for the management and treatment of psoriasis with biologics, J. Am. Acad. Dermatol. 80, 1029-1072 (2019).
9. D. Deleanu, I. Nedelea, Biological therapies for atopic dermatitis: An update (review), Exp. Ther. Med. 17, 1061-1067 (2019).
10. A. Jabbari, M. Suirez-Farinas, J. Fuentes-Duculan, J. Gonzalez, I. Cueto, A. G. Franks, J. G. Krueger, Dominant Th1 and minimal Th17 skewing in discoid lupus revealed by transcriptomic comparison with psoriasis, J. Invest. Dermatol. 134, 87-95 (2014).
11. B. F. Chong, L. chiang Tseng, G. A. Hosler, N. M. Teske, S. Zhang, D. R. Karp, N. J. Olsen, C. Mohan, A subset of CD163+ macrophages displays mixed polarizations in discoid lupus skin, Arthritis Res. Ther. 17, 324 (2015).
12. A. M. S. Barron, J. C. Mantero, J. D. Ho, B. Nazari, K. L. Horback, J. Bhawan, R. Lafyatis, C. Lam, J. L. Browning, Perivascular Adventitial Fibroblast Specialization Accompanies T Cell Retention in the Inflamed Human Dermis, J Immunol. 202, 56-68 (2019).
13. P. Mande, B. Zirak, W. C. Ko, K. Taravati, K. L. Bride, T. Y. Brodeur, A. Deng, K. Dresser, Z. Jiang, R. Ettinger, K. A. Fitzgerald, M. D. Rosenblum, J. E. Harris, A. Marshak-Rothstein, Fas ligand promotes an inducible TLR-dependent model of cutaneous lupus-like inflammation, J. Clin. Invest. 128, 2966-2978 (2018).
14. J. Liu, C. C. Berthier, J. M. Kahlenberg, Enhanced Inflammasome Activity in Systemic Lupus Erythematosus Is Mediated via Type I Interferon-Induced Up-Regulation of Interferon Regulatory Factor 1, Arthritis Rheumatol. 69, 1840-1849 (2017).
15. L. C. Tsoi, G. A. Hile, C. C. Berthier, M. K. Sarkar, T. J. Reed, J. Liu, R. Uppala, M. Patrick, K. Raja, X. Xing, E. Xing, K. He, J. E. Gudjonsson, J. M. Kahlenberg, Hypersensitive IFN Responses in Lupus Keratinocytes Reveal Key Mechanistic Determinants in Cutaneous Lupus, J. Immunol. 202, 2121-2130 (2019)
16. V. P. Werth, D. Fiorentino, B. A. Sullivan, M. J. Boedigheimer, K. Chiu, C. Wang, G. E. Arnold, M. A. Damore, J. Bigler, A. A. Welcher, C. B. Russell, D. A. Martin, J. B. Chung, Brief Report: Pharmacodynamics, Safety, and Clinical Efficacy of AMG 811, a Human Anti-Interferon-γ Antibody, in Patients With Discoid Lupus Erythematosus, Arthritis Rheumatol. 69, 1028-1034 (2017).
17. M. D. Catalina, P. Bachali, N. S. Geraci, A. C. Grammer, P. E. Lipsky, Gene expression analysis delineates the potential roles of multiple interferons in systemic lupus erythematosus, Commun. Biol. 2 (2019), doi: 10.1038/s42003-019-0382-x.
18. K. E. Nograles, L. C. Zaba, E. Guttman-Yassky, J. Fuentes-Duculan, M. Suirez-Farinas, I. Cardinale, A. Khatcherian, J. Gonzalez, K. C. Pierson, T. R. White, C. Pensabene, I. Coats, I. Novitskaya, M. A. Lowes, J. G. Krueger, Th17 cytokines interleukin (IL)-17 and IL-22 modulate distinct inflammatory and keratinocyte-response pathways, Br. J. Dermatol. 159, 1092-1102 (2008).
19. Y. Asano, Systemic sclerosis, J. Dermatol. 45, 128-138 (2018).
20. P. M. Brunner, E. Guttman-Yassky, D. Y. M. Leung, The immunology of atopic dermatitis and its reversibility with broad-spectrum and targeted therapies J. Allergy Clin. Immunol. 139, S65-S76 (2017).
21. K. M. Kingsmore, P. Bachali, M. D. Catalina, A. R. Daamen, S. E. Heuer, R. D. Robl, A. C. Grammer, P. E. Lipsky, Altered expression of genes controlling metabolism characterizes the tissue response to immune injury in lupus, Sci. Rep. 11 (2021), doi:10.1038/s41598-021-93034-w.
22. S. J. Waddell, S. J. Popper, K. H. Rubins, M. J. Griffiths, P. O. Brown, M. Levin, D. A. Relman, Dissecting Interferon-Induced Transcriptional Programs in Human Peripheral Blood Cells, PLoS One 5 (2010), doi:10.1371/journal.pone.0009753.
23. J. L. Sargent, A. Milano, S. Bhattacharyya, J. Varga, M. K. Connolly, H. Y. Chang, M. L. Whitfield, A TGFB-responsive gene signature is associated with a subset of diffuse scleroderma with increased disease severity, J. Invest. Dermatol. 130, 694-705 (2010).
24. C. L. Langrish, Y. Chen, W. M. Blumenschein, J. Mattson, B. Basham, J. D. Sedgwick, T. McClanahan, R. A. Kastelein, D. J. Cua, IL-23 drives a pathogenic T cell population that induces autoimmune inflammation, J. Exp. Med. 201, 233-240 (2005).
25. A. Blauvelt, A. Chiricozzi, The Immunologic Role of IL-17 in Psoriasis and Psoriatic Arthritis Pathogenesis, Clin. Rev. AllergyImmunol. 55, 379-390 (2018).
26. J. E. Hawkes, B. Y. Yan, T. C. Chan, J. G. Krueger, Discovery of the IL-23/IL-17 Signaling Pathway and the Treatment of Psoriasis, J. Immunol. 201, 1605-1613 (2018).
27. M. D. Catalina, P. Bachali, A. E. Yeo, N. S. Geraci, M. A. Petri, A. C. Grammer, P. E. Lipsky, Patient ancestry significantly contributes to molecular heterogeneity of systemic lupus erythematosus, JCI Insight 5 (2020), doi:10.1 172/jci.insight. 140380.
28. T. Vazquez, R. Feng, K. J. Williams, V. P. Werth, Immunological and clinical heterogeneityin cutaneous lupus erythematosus Br. J. Dermatol. 185, 481-483 (2021).
29. J. L. Zhu, L. T. Tran, M. Smith, F. Zheng, L. Cai, J. A. James, J. M. Guthridge, B. F. Chong, Modular gene analysis reveals distinct molecular signatures for subsets of patients with cutaneous lupus erythematosus, Br. J. Dermatol. (2021), doi:10.1111/bjd.19800.
30. L. C. Tsoi, E. Rodriguez, F. Degenhardt, H. Baurecht, U. Wehkamp, N. Volks, S. Szymczak, W. R. Swindell, M. K. Sarkar, K. Raja, S. Shao, M. Patrick, Y. Gao, R. Uppala, B. E. Perez White, S. Getsios, P. W. Harms, E. Maverakis, J. T. Elder, A. Franke, J. E. Gudjonsson, S. Weidinger, Atopic Dermatitis Is an IL-13-Dominant Disease with Greater Molecular Heterogeneity Compared to Psoriasis, J. Invest. Dermatol. 139, 1480-1489 (2019).
31. H. Valdimarsson, J. E. Gudjonsson, A. Johnston, H. Sigmundsdottir, H. Valdimarsson, Immunopathogenic mechanisms in psoriasis, Clin Exp Immunol 135, 1-8 (2004).
32. L. Pasquali, A. Srivastava, F. Meisgen, K. Das Mahapatra, P. Xia, N. Xu Landen, A. Pivarcsi, E. Sonkoly, The keratinocyte transcriptome in psoriasis: Pathways related to immune responses, cell cycle and keratinization, Acta Derm. Venereol. 99, 196-205 (2019).
33. M. B. Abdel-Naser, A. I. Liakou, R. Elewa, S. Hippe, J. Knolle, C. C. Zouboulis, Increased Activity and Number of Epidermal Melanocytes in Lesional Psoriatic Skin, Dermatology 232, 425-430 (2016).
34. H. Jin, M. K. Oyoshi, Y. Le, T. Bianchi, S. Koduru, C. B. Mathias, L. Kumar, S. Le Bras, D. Young, M. Collins, M. J. Grusby, J. Wenzel, T. Bieber, M. Boes, L. E. Silberstein, H. C. Oettgen, R. S. Geha, IL-21R is essential for epicutaneous sensitization and allergic skin inflammation in humans and mice, J. Clin. Invest. 119, 47-60 (2009).
35. F. GONG, Q. SU, Y. H. PAN, X. HUANG, W. H. SHEN, The emerging role of interleukin-21 in allergic diseases (Review), Biomed. Reports 1, 837-839 (2013).
36. A. P. Sappino, I. Masouye, J. H. Saurat, G. Gabbiani, Smooth muscle differentiation in scleroderma fibroblastic cells, Am. J. Pathol. 137, 585-591 (1990).
37. S. Domingo, C. Sole, T. Moline, B. Ferrer, J. Cortes-Hernindez, MicroRNAs in Several Cutaneous Autoimmune Diseases: Psoriasis, Cutaneous Lupus Erythematosus and Atopic Dermatitis Cells 9 (2020), doi:10.3390/cells9122656.
38. J. D'Orazio, S. Jarrett, A. Amaro-Ortiz, T. Scott, UV radiation and the skin, Int. J. Mol. Sci. 14, 12222-12248 (2013).
39. M. Laporte, P. Galand, D. Fokan, C. De Graef, M. Heenen, Apoptosis in established and healing psoriasis, Dermatology 200, 314-316 (2000).
40. A. Trautmann, M. Akdis, S. Klunker, K. Blaser, C. A. Akdis, Role of apoptosis in atopic dermatitis, Int. Arch. Allergy Immunol. 124, 230-232 (2001).
41. B. Franz, B. Fritzsching, A. Riehl, N. Oberle, C. D. Klemke, J. Sykora, S. Quick, C. Stumpf, M. Hartmann, A. Enk, T. Ruzicka, P. H. Krammer, E. Suri-Payer, A. Kuhn, Low number of regulatory T cells in skin lesions of patients with cutaneous lupus erythematosus, Arthritis Rheum. 56, 1910-1920 (2007).
42. A. H. Sawalha, K. M. Kaufman, J. A. Kelly, A. J. Adler, T. Aberle, J. Kilpatrick, E. K. Wakeland, Q. Z. Li, A. E. Wandstrat, D. R. Karp, J. A. James, J. T. Merrill, P. Lipsky, J. B. Harley, Genetic association of interleukin-21 polymorphisms with systemic lupus erythematosus, Ann. Rheum. Dis. 67, 458-461 (2008).
43. R. Webb, J. T. Merrill, J. A. Kelly, A. Sestak, K. M. Kaufman, C. D. Langefeld, J. Ziegler, P. Robert, J. C. Edberg, R. Ramsey-goldman, M. Petri, J. D. Reveille, G. S. Alarcón, L. M. Vili, M. E. Alarcón-Riquelme, J. A. James, G. S. Gilkeson, C. O. Jacob, K. L. Moser, P. M. Gaffney, T. J. Vyse, S. K. Nath, P. Lipsky, J. B. Harley, A. H. Sawalha, A polymorphism within interleukin-21receptor (IL21R) confers risk for systemic lupus erythematosus, Arthritis Rheumatol. 60, 2402-2407 (2009).
44. A. Puscas, A. Catana, C. Puscas, I. Roman, C. Vornicescu, M. Somlea, R. Orasan, Psoriasis: Association of interleukin-17 gene polymorphisms with severity and response to treatment (Review), Exp. Ther. Med., 875-880 (2019).
45. A. C. Allison Billi, F. Ma, O. Plazyo, M. Gharaee-Kermani, R. Wasikowski, G. A. Hile, X. Xing, C. M. Yee, S. M. Rizvi, M. P. Maz, F. Wen, L. C. Tsoi, M. Pellegrini, R. L. Modlin, J. E. Gudjonsson, J. Michelle Kahlenberg, A. C. Billi, F. Ma, O. Plazyo, M. Gharaee-Kermani, R. Wasikowski, G. A. Hile, X. Xing, C. M. Yee, S. M. Rizvi, M. P. Maz, F. Wen, L. C. Tsoi, M. Pellegrini, R. L. Modlin, J. E. Gudjonsson, J. Michelle Kahlenberg, Non-lesional and Lesional Lupus Skin Share Inflammatory Phenotypes that Drive Activation of CD16+ Dendritic Cells. BioRxiv (2021), doi:10.1101/2021.09.17.460124.
46. E. Der, H. Suryawanshi, P. Morozov, M. Kustagi, B. Goilav, S. Ranabathou, P. Izmirly, R. Clancy, H. M. Belmont, M. Koenigsberg, M. Mokrzycki, H. Rominieki, J. A. Graham, J. P. Rocca, N. Bornkamp, N. Jordan, E. Schulte, M. Wu, J. Pullman, K. Slowikowski, S. Raychaudhuri, J. Guthridge, J. James, J. Buyon, T. Tuschl, C. Putterman, J. Anolik, W. Apruzzese, A. Arazi, C. Berthier, M. Brenner, J. Buyon, R. Clancy, S. Connery, M. Cunningham, M. Dall'Era, A. Davidson, E. Der, A. Fava, C. Fonseka, R. Furie, D. Goldman, R. Gupta, J. Guthridge, N. Hacohen, D. Hildeman, P. Hoover, R. Hsu, J. James, R. Kado, K. Kalunian, D. Kamen, M. Kretzler, H. Maecker, E. Massarotti, W. McCune, M. McMahon, M. Park, F. Payan-Schober, W. Pendergraft, M. Petri, M. Pichavant, C. Putterman, D. Rao, S. Raychaudhuri, K. Slowikowski, H. Suryawanshi, T. Tuschl, P. Utz, D. Waguespack, D. Wofsy, F. Zhang, Tubular cell and keratinocyte single-cell transcriptomics applied to lupus nephritis reveal type I IFN and fibrosis relevant pathways, Nat. Immunol. (2019), doi:10.1038/s41590-019-0386-1.
47. T. M. Li, K. R. Veiga, N. Schwartz, Y. Chinenov, D. J. Oliver, J. Lora, A. Jabbari, Y. Liu, W. D. Shipman, M. J. Sandoval, I. F. Sollohub, W. G. Ambler, M. Rashighi, J. G. Krueger, N. Anandasabapathy, C. P. Blobel, T. T. Lu, Type I interferon modulates Langerhans cell ADAM17 to promote photosensitivity in lupus, BioRxiv (2021), doi: 10.1101/2021.08.18.456792.
48. K. A. Kirou, C. Lee, S. George, K. Louca, M. G. E. Peterson, M. K. Crow, Activation of the interferon-α pathway identifies a subgroup of systemic lupus erythematosus patients with distinct serologic features and active disease, Arthritis Rheum. 52, 1491-1503 (2005).
49. Q. Z. Li, J. Zhou, Y. Lian, B. Zhang, V. K. Branch, F. Carr-Johnson, D. R. Karp, C. Mohan, E. K. Wakeland, N. J. Olsen, Interferon signature gene expression is correlated with autoantibody profiles in patients with incomplete lupus syndromes, Clin. Exp. Immunol. 159, 281-291 (2010).
50. T. Iwamoto, J. Dorschner, M. Jolly, X. Huang, T. B. Niewold, Associations between type I interferon and antiphospholipid antibody status differ between ancestral backgrounds, Lupus Sci. Med. 5, 1-4 (2018).
51. C. C. Berthier, L. C. Tsoi, T. J. Reed, J. N. Stannard, E. M. Myers, R. Namas, X. Xing, S. Lazar, L. Lowe, M. Kretzler, J. E. Gudjonsson, J. M. Kahlenberg, Molecular Profiling of Cutaneous Lupus Lesions Identifies Subgroups Distinct from Clinical Phenotypes, J. Clin. Med. 8, 1244 (2019).
52. L. E. Tomalin, C. B. Russell, S. Garcet, D. A. Ewald, P. Klekotka, A. Nirula, H. Norsgaard, M. Sunrez-Farinas, J. G. Krueger, Short-term transcriptional response to IL-17 receptor-Aantagonism in the treatment of psoriasis, J. Allergy Clin. Immunol. 145, 922-932 (2020).
53. M. Robert, P. Miossec, Interleukin-17 and lupus: enough to be a target? For which patients?, Lupus 29, 6-14 (2020).
54. B. Ungar, A. B. Pavel, R. Li, G. Kimmel, J. Nia, P. Hashim, H. J. Kim, M. Chima, A. S. Vekaria, Y. Estrada, H. Xu, X. Peng, G. K. Singer, D. Baum, Y. Mansouri, M. Taliercio, E. Guttman-Yassky, Phase 2 randomized, double-blind study of IL-17 targeting with secukinumab in atopic dermatitis, J. Allergy Clin. Immunol. 147, 394-397 (2021).
55. A Study to Assess the Safety and Efficacy of Secukinumab in Alleviating Symptoms of Discoid Lupus Erythematosus, U.S. Natl. Libr. Med. Clin. Trials (2021), doi:10.31525/ct1-nct03866317.
56. S. Tyring, A. Gottlieb, K. Papp, K. Gordon, C. Leonardi, A. Wang, D. Lalla, M. Woolley, A. Jahreis, R. Zitnik, D. Cella, R. Krishnan, Etanercept and clinical outcomes, fatigue, and depression in psoriasis: double-blind placebo-controlled randomisedphase III trial, Lancet 367, 29-35 (2006).
57. K. Reich, F. O. Nestle, K. Papp, J. P. Ortonne, R. Evans, C. Guzzo, S. Li, L. T. Dooley, C. E. M. Griffiths, Infliximab induction and maintenance therapy for moderate-to-severe psoriasis: A phase III, multicentre, double-blind trial, Lancet 366, 1367-1374 (2005).
58. A. Menter, S. K. Tyring, K. Gordon, A. B. Kimball, C. L. Leonardi, R. G. Langley, B. E. Strober, M. Kaul, Y. Gu, M. Okun, K. Papp, Adalimumab therapy for moderate to severe psoriasis: A randomized, controlled phase III trial, J Am. Acad. Dermatol. 58, 106-115 (2008).
59. A. Blauvelt, K. Reich, M. Lebwohl, D. Burge, C. Arendt, L. Peterson, J. Drew, R. Rolleri, A. B. Gottlieb, Certolizumab pegol for the treatment of patients with moderate-to-severe chronic plaque psoriasis: pooled analysis of week 16 data from three randomized controlled trials, J. Eur. Acad. Dermatology Venereol. 33, 546-552 (2019).
60. A. Lorenzo-Vizcaya, D. A. Isenberg, The use of anti-TNF-alpha therapies for patients with systemic lupus erythematosus. Where are we now?, Expert Opin. Biol. Ther. 21, 639-647 (2021).
61. A. Jacobi, C. Antoni, B. Manger, G. Schuler, M. Hertl, Infliximab in the treatment of moderate to severe atopic dermatitis, J. Am. Acad. Dermatol. 52, 522-526 (2005).
62. N. Cassano, F. Loconsole, C. Coviello, G. A. Vena, CASE REPORT INFLIXIMAB IN RECALCITRANT SEVERE ATOPIC ECZEMA ASSOCIATED WITH CONTACT ALLERGY (2006).
63. M. Yuzaiful, M. Yusof, M. Wittmann, C. Fernandez, D. Wilson, S. Edward, G. Abignano, A. Alase, P. Laws, M. Goodfield, P. Emery, E. Vita, TARGETED THERAPY USING INTRADERMAL INJECTION OF ETANERCEPT FOR REMISSION INDUCTION IN DISCOID LUPUS ERYTHEMATOSUS (TARGET-DLE): RESULTS FROM A PROOF—OF-CONCEPT PHASE II TRIAL, Lupus Sci. Med. 6, A1-A227 (2019).
64. Targeted Therapy Using Intradermal Injection of Etanercept for Remission Induction in Discoid Lupus Erythematosus (TARGET-DLE), U.S. Natl. Libr. Med. Clin. Trials (2019).
65. A. B. Gottlieb, A. M. Goldminz, Ustekinumab for psoriasis and psoriatic arthritis, J. Rheumatol. 39, 86-89 (2012).
66. A Study of Ustekinumab in Participants With Active Systemic Lupus Erythematosus (2021;https://clinicaltrials.gov/ct2/show/NCT03517722?term=ustekinumab&cond=lupus&draw=2& rank=3).
67. Janssen Pharmaceuticals, Janssen announces discontinuation of Phase 3 LOTUS studyevaluating Ustekinumab in systemic lupus erythematosus, Janssen Pharm. (2020) (available at https://www.jnj.com/janssen-announces-discontinuation-of-phase-3-lotus-study-evaluating-ustekinumab-in-systemic-lupus-erythematosus).
68. R. F. van Vollenhoven, B. H. Hahn, G. C. Tsokos, C. L. Wagner, P. Lipsky, Z. Touma, V. P. Werth, R. M. Gordon, B. Zhou, B. Hsu, M. Chevrier, M. Triebel, J. L. Jordan, S. Rose, Efficacy and safety of ustekinumab, an IL-12 and IL-23 inhibitor, in patients with active systemic lupus erythematosus: results of a multicentre, double-blind, phase 2, randomised, controlled study, Lancet 392, 1330-1339 (2018).
69. W. D. Shipman, S. Chyou, A. Ramanathan, P. M. Izmirly, S. Sharma, T. Pannellini, D. C. Dasoveanu, X. Qing, C. M. Magro, R. D. Granstein, M. A. Lowes, E. G. Pamer, D. H. Kaplan, J. E. Salmon, B. J. Mehrara, J. W. Young, R. M. Clancy, C. P. Blobel, T. T. Lu, A protective Langerhans cell keratinocyte axis that is dysfunctional in photosensitivity, Sci. Transl. Med. 10 (2018), doi:10.1 126/scitranslmed.aap9527.
70. S. Tian, J. G. Krueger, K. Li, A. Jabbari, C. Brodmerkel, M. A. Lowes, M. Suirez-Farinas, Meta-Analysis Derived (MAD) Transcriptome of Psoriasis Defines the “Core” Pathogenesis of Disease, PLoS One 7 (2012), doi:10.1371/journal.pone.0044274.
71. L. M6bus, E. Rodriguez, I. Harder, D. Stolzl, N. Boraczynski, S. Gerdes, A. Kleinheinz, S. Abraham, A. Heratizadeh, C. Handrick, E. Haufe, T. Werfel, J. Schmitt, S. Weidinger, Atopic dermatitis displays stable and dynamic skin transcriptome signatures, J. Allergy Clin. Immunol. 147, 213-223 (2021).
72. L. C. Tsoi, M. T. Patrick, S. Shuai, M. K. Sarkar, S. Chi, B. Ruffino, A. C. Billi, X. Xing, R. Uppala, C. Zang, J. Fullmer, Z. He, E. Maverakis, N. N. Mehta, B. E. Perez White, S. Getsios, Y. Helfrich, J. J. Voorhees, J. M. Kahlenberg, S. Weidinger, J. E. Gudjonsson, Cytokine responses in nonlesional psoriatic skin as clinical predictor to anti-TNF agents, J. Allergy Clin. Immunol. 75, 15-18 (2021).
73. M. K. Sarkar, G. A. Hile, L. C. Tsoi, X. Xing, J. Liu, Y. Liang, C. C. Berthier, W. R. Swindell, M. T. Patrick, S. Shao, P. S. Tsou, R. Uppala, M. A. Beamer, A. Srivastava, S. L. Bielas, P. W. Harms, S. Getsios, J. T. Elder, J. J. Voorhees, J. E. Gudjonsson, J. M. Kahlenberg, Photosensitivity and type i IFN responses in cutaneous lupus are driven by epidermal-derived interferon kappa, Ann. Rheum. Dis. 77, 1653-1664 (2018).
74. A. C. Billi, M. Gharaee-Kermani, J. Fullmer, L. C. Tsoi, B. D. Hill, D. Gruszka, J. Ludwig, X. Xing, S. Estadt, S. J. Wolf, S. M. Rizvi, C. C. Berthier, J. B. Hodgin, M. A. Beamer, M. K. Sarkar, Y. Liang, R. Uppala, S. Shao, C. Zeng, P. W. Harms, M. E. Verhaegen, J. J. Voorhees, F. Wen, N. L. Ward, A. A. Dlugosz, J. M. Kahlenberg, J. E. Gudjonsson, The female-biased factor VGLL3 drives cutaneous and systemic autoimmunity, JCI Insight 4 (2019), doi: 10.1 172/jci.insight. 127291.
75. C. Brodmerkel, K. Li, S. Garcet, K. Hayden, A. Chiricozzi, I. Novitskaya, J. Fuentes-Duculan, M. Suarez-Farinas, K. Campbell, J. G. Krueger, Modulation of inflammatory gene transcripts in psoriasis vulgaris: Differences between ustekinumab and etanercept, J. Allergy Clin. Immunol. 143, 1962-1965 (2019).
76. E. Guttman-Yassky, R. Bissonnette, B. Ungar, M. Suirez-Farinas, M. Ardeleanu, H. Esaki, M. Suprun, Y. Estrada, H. Xu, X. Peng, J. I. Silverberg, A. Menter, J. G. Krueger, R. Zhang, U Chaudhry, B. Swanson, N. M. H. Graham, G. Pirozzi, G. D. Yancopoulos, J. D. Jennifer, Dupilumab progressively improves systemic and cutaneous abnormalities in patients with atopic dermatitis, J. Allergy Clin. Immunol. 143, 155-172 (2019).
77. S. Assassi, W. R. Swindell, M. Wu, F. D. Tan, D. Khanna, D. E. Furst, D. P. Tashkin, R. R. Jahan-Tigh, M. D. Mayes, J. E. Gudjonsson, J. T. Chang, Dissecting the heterogeneity of skin gene expression patterns in systemic sclerosis, Arthritis Rheumatol. 67, 3016-3026 (2015).
78. B. Skaug, D. Khanna, W. R. Swindell, M. E. Hinchcliff, T. M. Frech, V. D. Steen, F. N. Hant, J. K. Gordon, A. A. Shah, L. Zhu, J. Zheng, J. L. Browning, A. M. S. S. Barron, M. Wu, S. Visvanathan, P. Baum, J. M. Franks, M. L. Whitfield, V. K. Shanmugam, R. T. Domsic, F. V. Castelino, E. J. Bernstein, N. Wareing, M. A. Lyons, J. Ying, J. Charles, M. D. Mayes, S. Assassi, Global skin gene expression analysis of early diffuse cutaneous systemic sclerosis shows a prominent innate and adaptive inflammatory profile, Ann Rheum Dis 79, 379-386 (2020).
79. E. Eisenberg, E. Y. Levanon, Human housekeeping genes, revisited Trends Genet. 29, 569-574 (2013).
80. A. M. Bolger, M. Lohse, B. Usadel, Trimmomatic: A flexible trimmer for Illumina sequence data, Bioinformatics 30, 2114-2120 (2014).
81. A. Dobin, C. A. Davis, F. Schlesinger, J. Drenkow, C. Zaleski, S. Jha, P. Batut, M. Chaisson, T. R. Gingeras, STAR: Ultrafast universal RNA-seq aligner, Bioinformatics 29, 15-21 (2013).
82. A. Tarasov, A. J. Vilella, E. Cuppen, I. J. Nijman, P. Prins, Sambamba: fast processing of NGS alignment formats, Bioinformatics 31, 2032-2034 (2015).
83. Y. Liao, G. K. Smyth, W. Shi, FeatureCounts: An efficient general purpose program for assigning sequence reads to genomic features, Bioinformatics 30, 923-930 (2014).
84. Y. Liao, G. K. Smyth, W. Shi, The Subread aligner: Fast, accurate and scalable read mapping by seed-and-vote, Nucleic Acids Res. 41 (2013), doi:10.1093/nar/gkt214.
85. M. I. Love, W. Huber, S. Anders, Moderated estimation of fold change and dispersion for RNA-seq data with DESeq2, Genome Biol. 15, 1-21 (2014).
86. S. Hanzelmann, R. Castelo, J. Guinney, GSVA: gene set variation analysis for microarray and RNA-Seq data (2013; http://www.biomedcentral.com/1471-2105/14/7http://www.bioconductor.org.Background).
87. C. Cheadle, M. P. Vawter, W. J. Freed, K. G. Becker, Analysis ofMicroarray Data Using Z Score Transformation (http://www.grc.nia.nih.gov/).
88. J. Menche, E. Guney, A. Sharma, P. J. Branigan, M. J. Loza, F. Baribaud, R. Dobrin, A. L. Barabisi, Integrating personalized gene expression profiles into predictive disease-associated gene pools, npj Syst. Biol. Appl. 3 (2017), doi:10.1038/s41540-017-0009-0.
89. L. Fagerberg, B. M. Hallstrom, P. Oksvold, C. Kampf, D. Djureinovic, J. Odeberg, M. Habuka, S. Tahmasebpoor, A. Danielsson, K. Edlund, A. Asplund, E. Sjostedt, E. Lundberg, C. A. K. Szigyarto, M. Skogs, J. Ottosson Takanen, H. Berling, H. Tegel, J. Mulder, P. Nilsson, J. M. Schwenk, C. Lindskog, F. Danielsson, A. Mardinoglu, A. Sivertsson, K. Von Feilitzen, M. Forsberg, M. Zwahlen, I. Olsson, S. Navani, M. Huss, J. Nielsen, F. Ponten, M. Uhlen, Analysis of the human tissue-specific expression by genome-wide integration of transcriptomics and antibody-based proteomics, Mol. Cell. Proteomics 13, 397-406 (2014).
90. M. Uhlen, L. Fagerberg, B. M. Hallstrom, C. Lindskog, P. Oksvold, A. Mardinoglu, A. Sivertsson, C. Kampf, E. Sjostedt, A. Asplund, I. M. Olsson, K. Edlund, E. Lundberg, S. Navani, C. A. K. Szigyarto, J. Odeberg, D. Djureinovic, J. O. Takanen, S. Hober, T. Alm, P. H. Edqvist, H. Berling, H. Tegel, J. Mulder, J. Rockberg, P. Nilsson, J. M. Schwenk, M. Hamsten, K. Von Feilitzen, M. Forsberg, L. Persson, F. Johansson, M. Zwahlen, G. Von Heijne, J. Nielsen, F. Pontén, Tissue-based map of the human proteome, Science (80-.). 347, 394 (2015)
91. A. Gazel, P. Ramphal, M. Rosdy, B. De Wever, C. Torier, N. Hosein, B. Lee, M. Tomic-Canic, M. Blumenberg, Transcriptional Profiling of Epidermal Keratinocytes: Comparison of Genes Expressed in Skin, Cultured Keratinocytes, and Reconstituted Epidermis, Using Large DNA Microarrays, J. Invest. Dermatol. 121, 1459-1468 (2003).
92. Q. Xu, P. K. Majumder, K. Ross, Y. Shim, T. R. Golub, M. Loda, W. R. Sellers, Identification of prostate cancer modifier pathways using parental strain expression mapping, Proc. Natl. Acad. Sci. U.S.A 104, 17771-17776 (2007).
93. A. T. J. Wierenga, A. Cunningham, A. Erdem, N. V. Lopera, A. Z. Brouwers-Vos, M. Pruis, A. B. Mulder, U. L. Gunther, J. H. A. Martens, E. Vellenga, J. J. Schuringa, HIF1/2-exerted control over glycolytic gene expression is not functionally relevant for glycolysis in human leukemic stem/progenitor cells, Cancer Metab. 7, 1-17 (2019).
94. R. Mansourian, D. M. Mutch, N. Antille, J. Aubert, P. Fogel, J. M. Le Goff, J. Moulin, A. Petrov, A. Rytz, J. J. Voegel, M. A. Roberts, The Global Error Assessment (GEA) model for the selection of differentially expressed genes in microarray data, Bioinformatics 20, 2726-2737 (2004).
95. T. Banno, A. Gazel, M. Blumenberg, Pathway-specific profiling identifies the NF-κB-dependent tumor necrosis factor α-regulated genes in epidermal keratinocytes, J. Biol. Chem. 280, 18973-18980 (2005).
96. K. Wolk, E. Witte, E. Wallace, W. D. Döcke, S. Kunz, K. Asadullah, H. D. Volk, W. Sterry, R. Sabat, IL-22 regulates the expression of genes responsible for antimicrobial defense, cellular differentiation, and mobility in keratinocytes: A potential role in psoriasis, Eur. J. Immunol. 36, 1309-1323 (2006).
97. C. F. Cheng, J. Fan, B. Bandyopahdhay, D. Mock, S. Guan, M. Chen, D. T. Woodley, W. Li, Profiling motility signal-specific genes in primary human keratinocytes, J. Invest. Dermatol. 128, 1981-1990 (2008).
98. F. Shen, S. L. Gaffen, Structure-function relationships in the IL-17 receptor: Implications for signal transduction and therapy Cytokine 41, 92-104 (2008).
99. S. Yano, T. Banno, R. Walsh, M. Blumenberg, Transcriptional responses of human epidermal keratinocytes to cytokine interleukin-1, J. Cell. Physiol. 214, 1-13 (2008).
100. Y. Yao, L. Richman, C. Morehouse, M. de los Reyes, B. W. Higgs, A. Boutrin, B. White, A. Coyle, J. Krueger, P. A. Kiener, B. Jallal, Type I interferon: Potential therapeutic target for psoriasis?, PLoS One 3 (2008), doi:10.1371/journal.pone.0002737.
101. M. Suirez-Farinas, M. A. Lowes, L. C. Zaba, J. G. Krueger, Evaluation of the Psoriasis Transcriptome across Different Studies by Gene Set Enrichment Analysis (GSEA), PLoS One 5 (2010), doi:10.1371/journal.pone.0010247.
102. A. Chiricozzi, E. Guttman-Yassky, M. Suirez-Farias, K. E. Nograles, S. Tian, I. Cardinale, S. Chimenti, J. G. Krueger, Integrative responses to IL-17 and TNF-α in human keratinocytes account for key inflammatory pathogenic circuits in psoriasis, J. Invest. Dermatol. 131, 677-687 (2011).
103. H. Fujita, The role of IL-22 and Th22 cells in human skin diseases, J. Dermatol. Sci. 72, 3-8 (2013).
104. S. Hirakawa, R. Saito, H. Ohara, R. Okuyama, S. Aiba, Dual Oxidase 1 Induced by Th2 Cytokines Promotes STAT6 Phosphorylation via Oxidative Inactivation of Protein Tyrosine Phosphatase 1B in Human Epidermal Keratinocytes, J. Immunol. 186, 4762-4770 (2011).
105. V. Ramirez-Carrozzi, A. Sambandam, E. Luis, Z. Lin, S. Jeet, J. Lesch, J. Hackney, J. Kim, M. Zhou, J. Lai, Z. Modrusan, T. Sai, W. Lee, M. Xu, P. Caplazi, L. Diehl, J. De Voss, M. Balazs, L. Gonzalez, H. Singh, W. Ouyang, R. Pappu, IL-17C regulates the innate immune function of epithelial cells in an autocrine manner, Nat. Immunol. 12, 1159-1166 (2011).
106. W. R. Swindell, A. Johnston, J. J. Voorhees, J. T. Elder, J. E. Gudjonsson, Dissecting the psoriasis transcriptome: Inflammatory- and cytokine-driven gene expression in lesions from 163 patients, BMC Genomics 14 (2013), doi:10.1186/1471-2164-14-527.
107. B. Li, L. C. Tsoi, W. R. Swindell, J. E. Gudjonsson, T. Tejasvi, A. Johnston, J. Ding, P. E. Stuart, X. Xing, J. J. Kochkodan, J. J. Voorhees, H. M. Kang, R. P. Nair, G. R. Abecasis, J. T. Elder, Transcriptome analysis of psoriasis in a large case-control sample: RNA-seq provides insights into disease mechanisms, J. Invest. Dermatol. 134, 1828-1838 (2014).
108. T. Duhen, C. Ni, D. Campbell, Identification of a specific gene signature in human Th1/17 cells (BA13P.126), J. Immunol. 192, 177.12 LP-177.12 (2014).
109. B. Hollbacher, T. Duhen, S. Motley, M. M. Klicznik, I. K. Gratz, D. J. Campbell, Transcriptomic profiling of human effector and regulatory T cell subsets identifies predictive population signatures, Immunohorizons 4, 585-596 (2021).
110. J. B. Wing, Y. Kitagawa, M. Locci, H. Hume, C. Tay, T. Morita, Y. Kidani, K. Matsuda, T. Inoue, T. Kurosaki, S. Crotty, C. Coban, N. Ohkura, S. Sakaguchi, A distinct subpopulation of CD25− T-follicular regulatory cells localizes in the germinal centers, Proc. Natl. Acad. Sci. U.S.A 114, E6400-E6409 (2017).
111. N. Kutukculer, E. Azarsiz, G. Aksu, N. E. Karaca, CD4+CD25+Foxp3+ T regulatory cells, Th1 (CCR5, IL-2, IFN-γ) and Th2 (CCR4, IL-4, 11-13) type chemokine receptors and intracellular cytokines in children with common variable immunodeficiency, Int. J. Immunopathol. Pharmacol.29, 241-251 (2016).
112. L. Breiman, J. H. Friedman, R. A. Olshen, C. J. Stone, Classification and Regression Trees (1984).
113. L. Breiman, Random forests, Mach. Learn. 45, 5-32 (2001).
114. R. Blagus, L. Lusa, SMOTE for high-dimensional class-imbalanced data, BMC Bioinformatics 14 (2013), doi:10.1186/1471-2105-14-106.
115. L. V. Hedges, Distribution Theory for Glass's Estimator of Effect size and Related Estimators, J. Educ. Stat. 6, 107-128 (1981).
116. Z. Gu, R. Eils, M. Schlesner, Complex heatmaps reveal patterns and correlations in multidimensional genomic data, Bioinformatics 32, 2847-2849 (2016).
117. B. A. Martinez, S Shrotri, K. Kingsmore, P. Bachali, A. Grammer, P. Lipsky, Machine learning reveals distinct gene signature profiles in lesional and nonlesional regions of inflammatory skin diseases, Science Advances 8, eabn4776 (29 Apr. 2022), including supplementary materials, incorporated by reference herein in its entirety.
118. Kingsmore, Kathrvn M., Prathyusha Bachali, Michelle D. Catalina, Andrea R. Daamen, Sarah E. Heuer, Robert D. Robl, Amrie C. Grammer, and Peter E. Lipsky. Altered expression of genes controlling metabolism characterizes the tissue response to immune injury in lupus. Scientific Reports 1, no. 1 (2021 Jul. 20): 14789, including supplementary materials, incorporated by reference herein in its entirety.
119. Kingsmore et al. Metabolic dysregulation characterizes the tissue response to immune injury in systemic lupus erythematosus and inflammatory skin diseases. Lupus 21st Century 2021; incorporated by reference herein in its entirety.
120. Martinez et al. Comparative analysis of inflammatory skin diseases reveals shared and distinct gene signature profiles in lesional and nonlesional regions. #507 Society for Investigative Dermatology annual meeting May 18-21, 2022: incorporated by reference herein in its entirety.

### Example 3: The Transcriptomic Landscape of Nephritic Kidneys Reveals Mechanisms for End Organ Resistance to Damage in Lupus-Prone Mice

Pathologic inflammation is a major driver of kidney damage in lupus nephritis (LN), but the immune mechanisms of disease progression and risk factors for end organ damage are poorly understood. To characterize molecular profiles through the development of LN, we carried out gene expression analysis of micro-dissected kidneys from lupus-prone NZM2328 mice. We identified a continuum of inflammatory processes associated with the progression from acute inflammatory to chronic destructive disease initiated in the glomeruli and progressing to the tubulointerstitium. We examined male mice and the congenic NZM2328.R27 strain, both of which are resistant to the development of chronic nephritis and end organ damage, as a means to define pathogenic processes. Male mice exhibited minimal immune infiltration in the glomeruli resulting in non-progressive renal pathology. Immune infiltrates in the glomeruli of R27 mice expressed a regulatory gene signature and especially a dominance of M2-like macrophages. Moreover, R27 mice manifested an enhanced kidney tubule signature, with evidence of increased mitochondrial activity, glycolysis, and lipid metabolism, consistent with a functional resistance to cellular damage. The robust tubule signature was associated with the absence of an immune/inflammatory gene signature. Numerous genes in the R27 genetic region were upregulated in NZM2328 nephritic kidneys, that could contribute to the protective effect of this interval on the evolution of LN.

Systemic lupus erythematosus (SLE) is an autoimmune disorder that can affect a variety of tissues, including the kidney (1). Lupus nephritis (LN) is one of the most severe organ manifestations of SLE and affects approximately 40% of adult lupus patients with 10-20% of patients developing end-stage renal disease (ESRD) (2). Disease is thought to initiate in the kidney glomeruli with immune complex (IC) deposition and complement activation leading to the release of damage associated molecular patterns (DAMPS), cytokine production, and the infiltration of inflammatory cells that amplify and sustain inflammation (3, 4). Damage to the kidney glomeruli promotes ischemic damage and chronic hypoxia, compromising the downstream blood supply to the tubulointerstitium (TI) and reducing tubule cell viability, which serve as prognostic markers for the development of ESRD (5-7).

In current practice, the severity of lupus nephritis pathogenesis is determined by histological classification, which is used to drive therapeutic decisions and assess the potential for terminal kidney damage (8-10). However, despite advances in classification, there remains no clear indication of factors controlling the conversion of acute to chronic nephritis and no proven treatments to prevent ESRD (11-13). Therefore, there remains a need to understand the risk factors for chronic disease and the stages of inflammation leading to ESRD in greater detail.

Previous studies established the NZM2328 lupus-prone mouse strain as a model for human proliferative glomerulonephritis (GN) with severe IC-mediated nephritis and early mortality predominantly affecting female mice (14-16). These studies determined that disease in female NZM2328 mice presents in two stages termed acute GN (AGN) with pathology largely confined to the glomerulus, and chronic (GN) in which inflammation and tissue damage are also found amongst and between the tubules (15, 16). Each stage was associated with a genetic locus of chromosome 1 and the Agnz1 and Cgnz1 regions were found to be critical for the development of AGN and CGN respectively. In addition, the NZM2328.Lc1R27 (R27) recombinant strain was generated by replacing the Cgnz1 region of NZM2328 with that from the C57BL/J strain, such that female R27 mice develop AGN but do not progress to CGN. Similarly, male NZM2328 mice develop a milder, acute form of nephritis but do not exhibit severe proteinuria or progress to chronic disease (17, 18). Despite these clear clinical distinctions, the nature of the immune/inflammatory response driving AGN and the contribution of tissue as well as inflammatory cells and processes to the development of CGN resulting in renal failure and early death have not been described.

The heterogeneity in disease presentation among LN patients and difficulty in predicting therapeutic responses from histology alone have highlighted the utility of molecular profiling to improve classification of lupus kidney pathology (13, 19). Here, to understand the pathogenesis of lupus nephritis and especially the relationship between acute and chronic GN in greater detail, we utilized transcriptome analysis to define the stages of GN in NZM2328 mice and identify pathologic immune populations and processes associated with disease progression. In addition, we identified distinct mechanisms of resistance to chronic disease based on differences in gender and genetics with implications for elucidating risk factors for development of ESRD in human lupus patients.

### Results

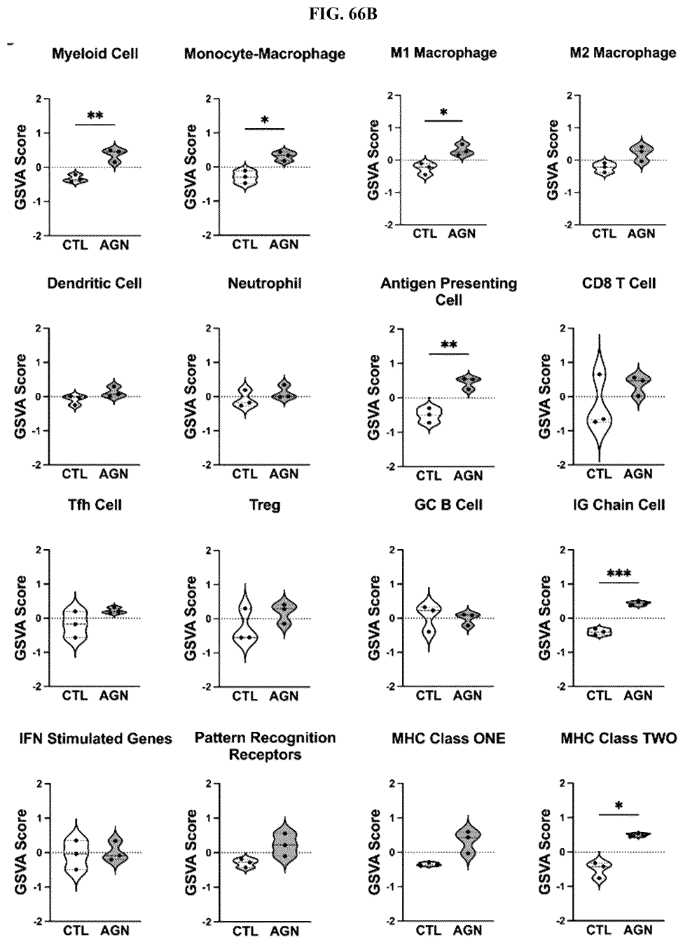

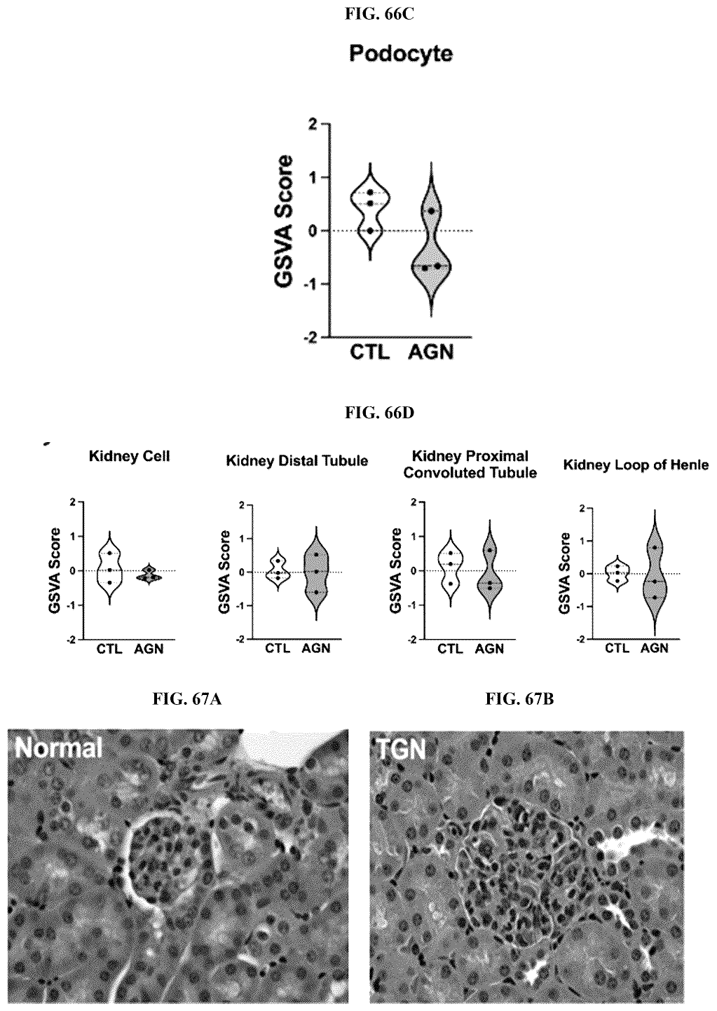

Transcriptional profiling uncovers immune populations present at the onset of GN in NZM2328 mice. To establish the inflammatory environment in the kidney at disease onset, we analyzed the transcriptomes of micro-dissected glomeruli and tubulointerstitial tissue from the kidneys of female NZM2328 mice. Tissues from 8-9 week-old (CTL) mice were used as a control for 36 week-old (AGN) mice as characterized in our previous studies (14-16). We carried out Gene Set Variation Analysis (GSVA) (20) using a set of curated gene sets to analyze gene expression from CTL and AGN mice and to identify immunologic gene signature enrichment at the acute stage (FIGS. 66A-D, Tables 19-1 to 19-36). The glomeruli of AGN mice were enriched for gene signatures of a number of pathologic immune cell types, including myeloid cells, M1 macrophages (Mϕs), antigen presenting cells (APCs), CD8 T cells, and T follicular helper (Tfh) cells (FIG. 66A). In addition, genes encoding immune cell receptors including pattern recognition receptors (PRRs) as well as major histocompatibility complex (MHC) class I and II were significantly elevated in AGN glomeruli. The tubulointerstitium (TI) of AGN mice was enriched for many of the same immune signatures including myeloid cells, M1 Mϕs, APCs, and MHC class II as well as the IG chain signature indicative of the presence of a plasma cell infiltrate (FIG. 66B). However, despite the presence of signatures indicative of immune cell infiltrates, gene signatures of podocytes in the glomeruli (FIG. 66C) and of tubule cells in the TI (FIG. 66D) were not significantly different than CTL. Thus, the kidneys of mice with AGN are enriched for pro-inflammatory, and predominantly innate immune cell gene signatures with no evidence of damage to the kidney cells.

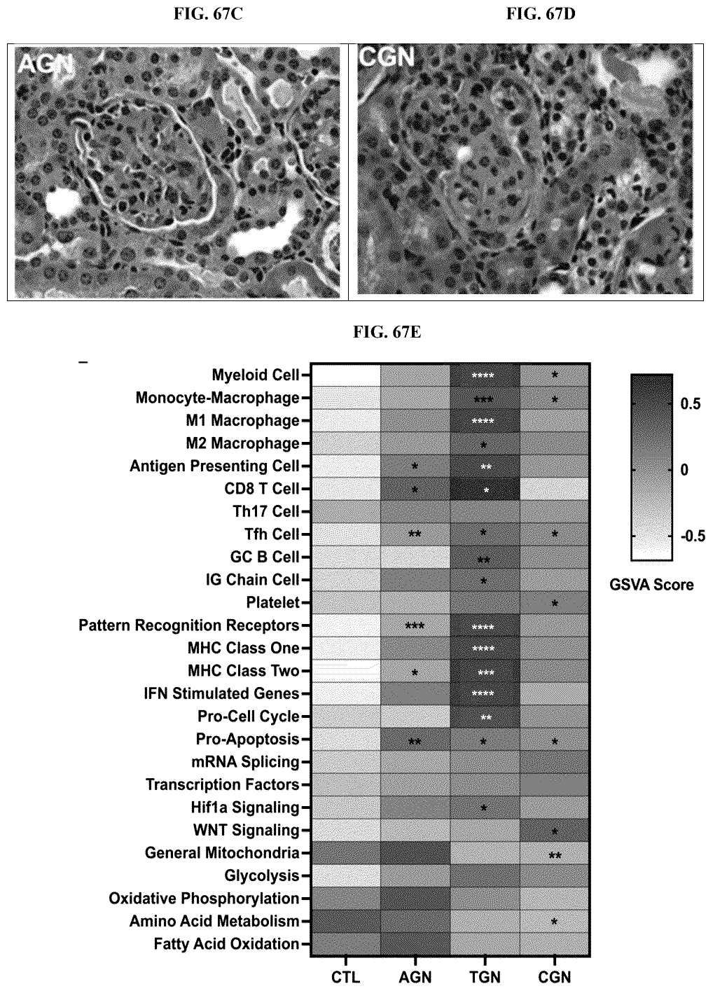

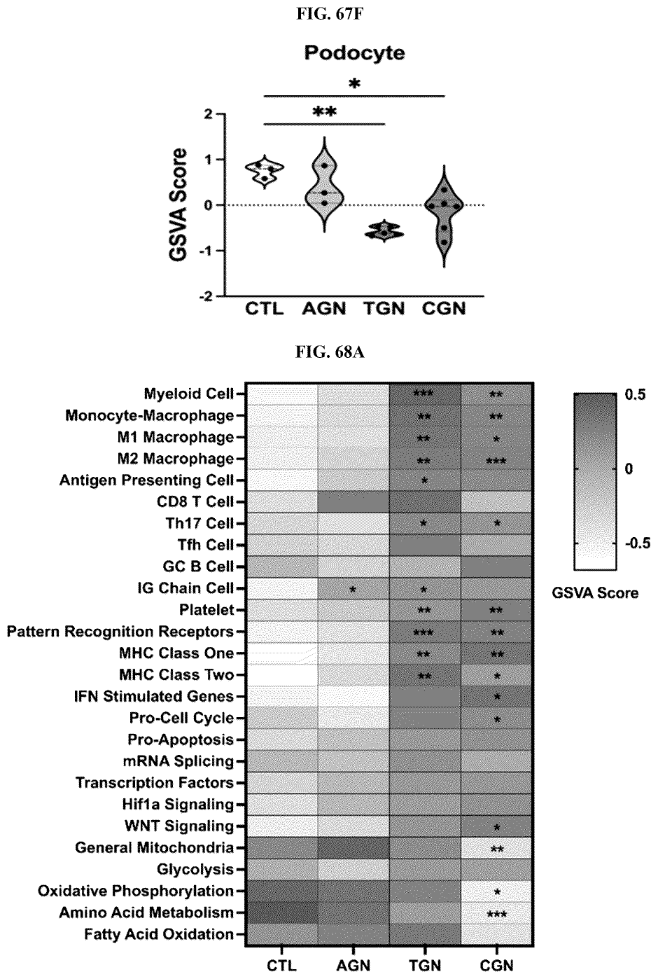

Renal disease of NZM2328 mice is characterized by escalating stages of inflammation. To identify different stages of GN in the kidneys of female NZM2328 mice, we carried out histological studies at regular intervals throughout disease progression (FIGS. 67A-D; Table 17). Tissues from young mice, before disease development and without evidence of kidney pathology were used as a control for diseased mice (FIG. 67A). At the AGN stage, glomeruli were increased in size with evidence of mesangial expansion, immune cell infiltration, and immune complex deposition including IgG, C3, and anti-nuclear antibody (ANA) deposits (FIG. 67B). There were no changes to tubule cells of AGN mice and they exhibited mild immune cell infiltration in the interstitium. We identified an intermediate stage of disease progression termed transitional GN (TGN) at which, like the AGN stage, glomeruli exhibited mesangial expansion and immune cell infiltration, but levels of IgG and C3 deposition as well as serum levels of anti-DNA antibodies were elevated over AGN mice (FIG. 67C). The interstitium of TGN mice had more inflammatory cells than at the AGN stage and tubular cells showed some dilation and atrophy. However, tubule damage was not evident. At the CGN stage, mice exhibited glomerular sclerosis, fibrosis with interstitial inflammation, and the highest level of immune complex deposition as compared to earlier disease stages (FIG. 67D). In CGN stage mice, >80% of tubular cells had tubular dilation with increased evidence of atrophy and tubular casts as compared to the TGN stage.

Transcriptomic analysis reveals distinct immune profiles of acute, transitional, and chronic GN in NZM2328 mice. To establish immune profiles of the stages of LN, we compared the transcriptomes of micro-dissected glomeruli and tubulointerstitial tissue from pre-disease CTL mice with mice with progressively more severe stages of disease (AGN: 36 weeks old; TGN: 36-37 weeks old; CGN: 39 weeks old). We carried out GSVA using a battery of informative gene sets to analyze gene expression from each cohort and to determine the immune cells and inflammatory pathways enriched through the development of CGN (FIGS. 67E-F & 68A-B). In the glomerulus, the AGN stage was characterized by enrichment of Tfh cell, APC, and CD8 T cell signatures in conjunction with PRR, MHC class II, and apoptosis gene signatures (FIG. 67E). No other immune/inflammatory pathways were significantly enriched when compared with later-stage cohorts. The immune profile of the glomerulus of TGN mice reflected an intermediate stage of renal disease and the peak of inflammatory signature enrichment. We found increased enrichment of gene signatures of germinal center (GC) B cells, myeloid cells, and Mϕs, including both M1 and M2 subsets, as well as signatures of interferon (IFN) stimulated genes, MHC class I, the cell cycle, and the Hifla signaling pathway (FIG. 67E). In addition, the inflammatory signatures enriched in AGN mice were increased further at the transitional stage. The glomeruli of CGN mice were enriched for platelets and WNT signaling and de-enriched for gene signatures of mitochondrial function and amino acid metabolism (FIG. 67E). Along with the enrichment of gene signatures of inflammatory cells and pathways, we also found evidence of kidney damage in TGN and CGN mice with de-enrichment of the gene signature for podocytes (FIG. 67F).

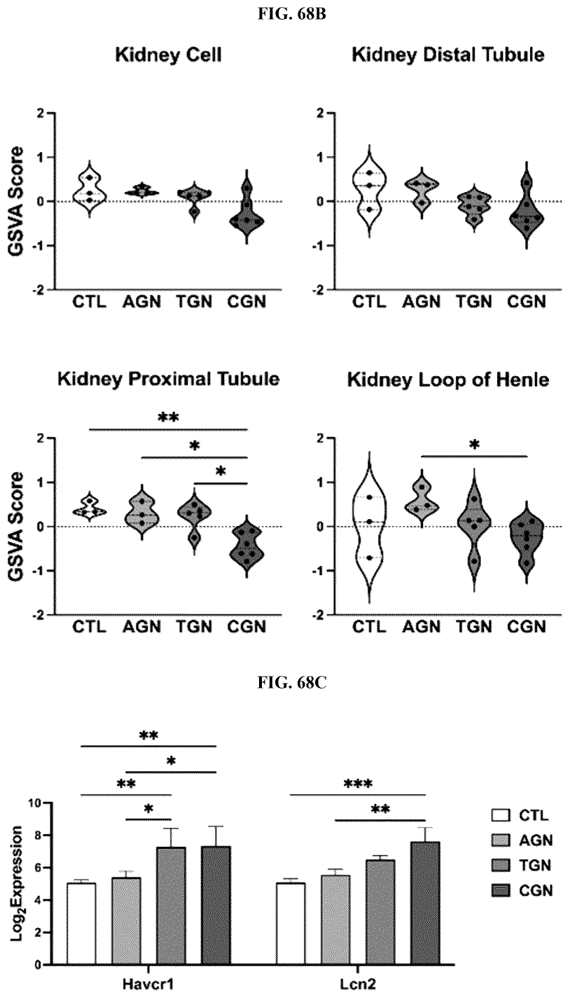

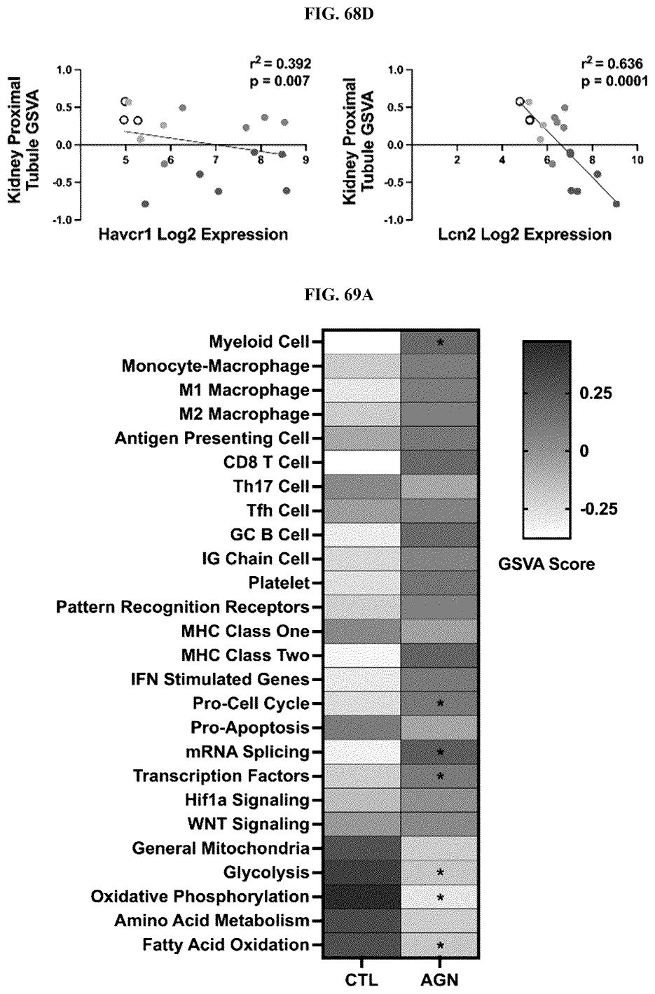

Gene expression results from the TI of NZM2328 kidneys showed a progressive pattern of inflammation and kidney cell loss. The TI of AGN showed no abnormalities except for an increase in IG chains, indicative of plasma cells, and no abnormalities in tissue cell gene expression. (FIGS. 68A&B). In contrast, The TI regions of TGN kidneys were enriched for numerous immune and inflammatory signatures, including Th17 cells, PRRs, MHC class I and II, myeloid cells, and M1 and M2 Mϕs (FIG. 68A). The TI of chronic stage mice exhibited enrichment of IFN stimulated genes, the cell cycle, and WNT signaling as well as decreases in gene signatures for mitochondria, amino acid metabolism, and oxidative phosphorylation (FIG. 68A). We found further indicators of damage to the kidney tubules in chronic stage mice with decreases in kidney tubule cell gene signatures (FIG. 68B) that correlated with significant increases in the expression of the kidney tubule damage-associated genes, Havcr1 and Lcn2 (FIGS. 68C&D). Overall, these results suggest that renal disease in female NZM2328 mice progresses from the glomerulus to the tubules and that inflammation established in the acute and transitional stages promotes a decrease in kidney cell signatures, indicative of cell damage.

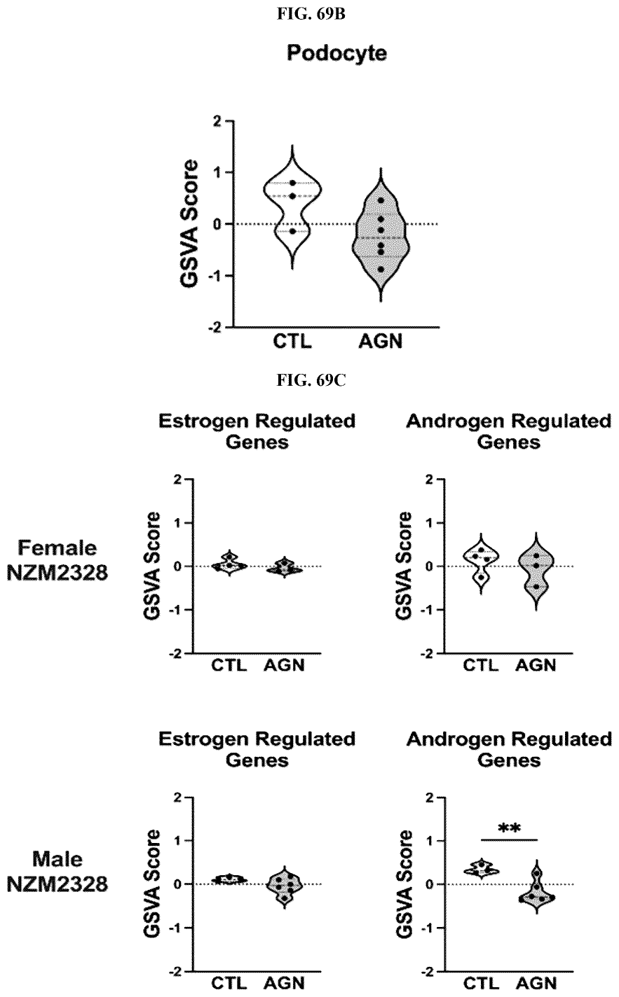

Lack of inflammatory signatures in the glomerulus of NZM2328 male mice associated with lack of progression to chronic renal disease. SLE in human patients exhibits sex bias and affects females nine times more frequently than males (21). This also occurs in the renal disease of NZM2328 mice as males develop acute GN with immune complex deposition in the kidneys, but rarely develop severe proteinuria and do not progress further to chronic disease (17, 18). Thus, to gain insight into the differences in the nature of disease in male NZM2328 mice, we evaluated the transcriptomes of glomeruli from male mice with AGN at 10 months of age as compared to pre-disease, 8-9-week-old mice (FIGS. 69A-C). Even though male mice were selected because they had evident immune complex deposition, in contrast to the females, male AGN mice were not enriched for gene signatures indicative of a robust adaptive immune response, myeloid cell infiltration in the kidney, or increased inflammation in the kidneys (FIG. 69A). Instead, males exhibited enrichment for signatures of mRNA splicing and transcription factors and de-enrichment of metabolic pathways, including glycolysis, oxidative phosphorylation, and fatty acid oxidation. In addition, there was no difference in expression of kidney tissue signatures in male mice with acute stage disease as compared to normal controls (FIG. 69B).

The increased rate of SLE in females over males implicates sex hormones and downstream cellular processes as potential contributors to disease (22, 23). To assess the role of sex hormones in renal disease in NZM2328 mice, we developed signatures of estrogen- and androgen-regulated genes and compared their enrichment in female and male mice with AGN as compared to normal controls (FIG. 69C). Female AGN mice exhibited no differences in enrichment of hormone-regulated gene signatures. However, in males, androgen-regulated genes were decreased in AGN mice. Furthermore, the majority of androgen-regulated genes contributing to the de-enrichment in male mice were related to mitochondrial function and metabolic pathways, including Akap1, Cox6b1, Iapp, Mrps6, Mybbp1a, Ndufa1, Phkg2, Prelid1, Sord, and Tmem86a. This result suggested that a decreased male hormone response in AGN mice may contribute to resistance to disease progression by regulating metabolism and dampening inflammation.

Inflammatory gene signatures in the glomeruli of R27 mice differ from those in NZM2328 mice. In addition to the differences in severity between males and females, there is also a genetic component to the risk for development of chronic renal disease in female NZM2328 mice. Previous work identified the CGN-associated locus, Cgnz1, and the R27 congenic mouse strain generated by replacing the Cgnz1 locus with that from the non-lupus prone C57BL/J mouse (15, 16). Female R27 mice develop AGN with similar kidney pathology to NZM2328 mice, but do not progress further to severe proteinuria and ESRD suggesting that they are resistant to kidney damage. To examine the basis of non-progressive GN in R27 mice, we analyzed gene expression data from the glomeruli and tubulointerstitial tissue of normal, control R27 mice (8-9 weeks) and R27 mice with AGN (12 months). R27 mice with AGN were identified by the presence of proteinuria and glomerular deposits of immunoglobulin detected by immunofluorescence

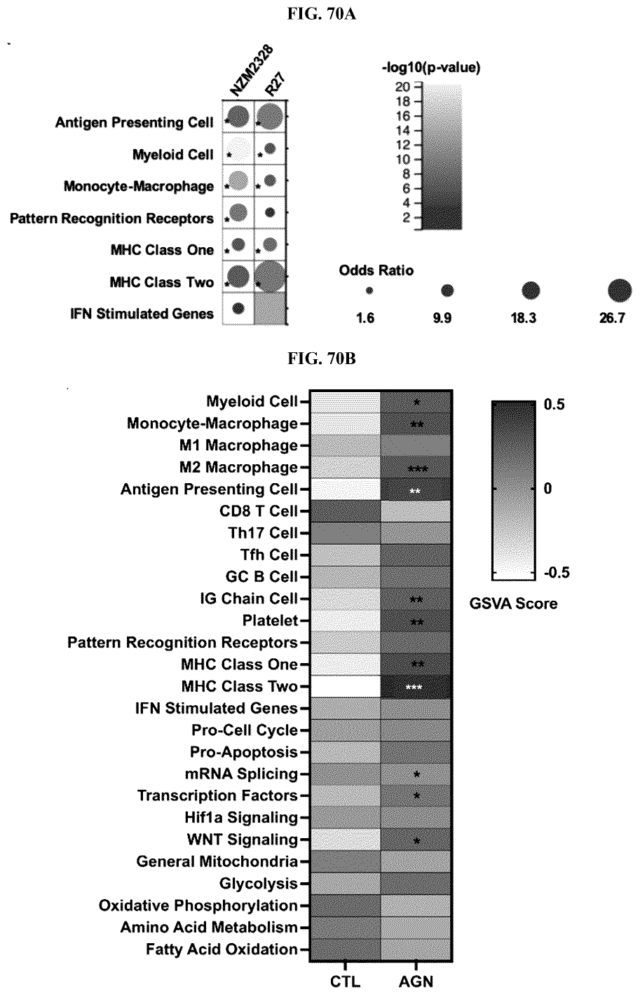

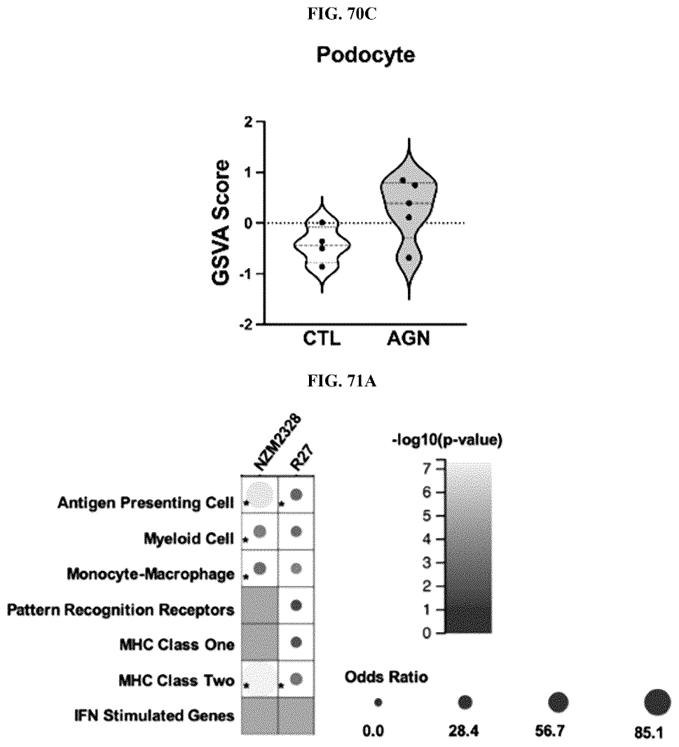

First, we assessed differentially expressed genes (DEGs) from the glomeruli of NZM2328 (Table 21) and R27 mice (Table 22) relative to their respective CTLs to compare the inflammatory environment of R27 mice directly with that of the parent strain that develops CGN (FIG. 70A). DEGs from the glomeruli of both NZM2328 and R27 AGN mice exhibited significant overlaps with increased expression of APC, myeloid cell, M$, and MHC signatures. Notably, the myeloid cell and M$ signatures exhibited more significant overlap with DEGs from NZM2328 mice. In addition, DEGs from NZM2328 but not R27 AGN mice were associated with PRRs and IFN stimulated genes, suggesting a more severe inflammatory environment than in the glomeruli of the congenic R27 mice with acute disease.

We next carried out GSVA to determine the relative enrichment of gene signatures for immune and inflammatory cells and pathways in R27 AGN mice as compared to R27 CTL mice (FIGS. 70B & 71B). The glomeruli of R27 AGN mice were enriched for gene signatures indicative of inflammation, including APCs, IG Chains, Mϕs, and MHC class I and II (FIG. 70B). However, separate analysis of M$ subsets revealed that a gene signature of anti-inflammatory M2, but not pro-inflammatory M1 Mϕs was enriched in R27 mice. Notably, there was no difference in IFN stimulated genes, PRRs, or Hifla signaling between R27 control and AGN mice. Furthermore, there was no evidence of kidney damage, as kidney cell-specific gene signatures were also unchanged in R27 AGN mice (FIG. 70C). Overall, despite the increase in inflammatory cell and pathway gene signatures in R27 AGN mice, the differences in the nature of this inflammation from NZM2328 female mice may contribute to resistance from progression to chronic disease.

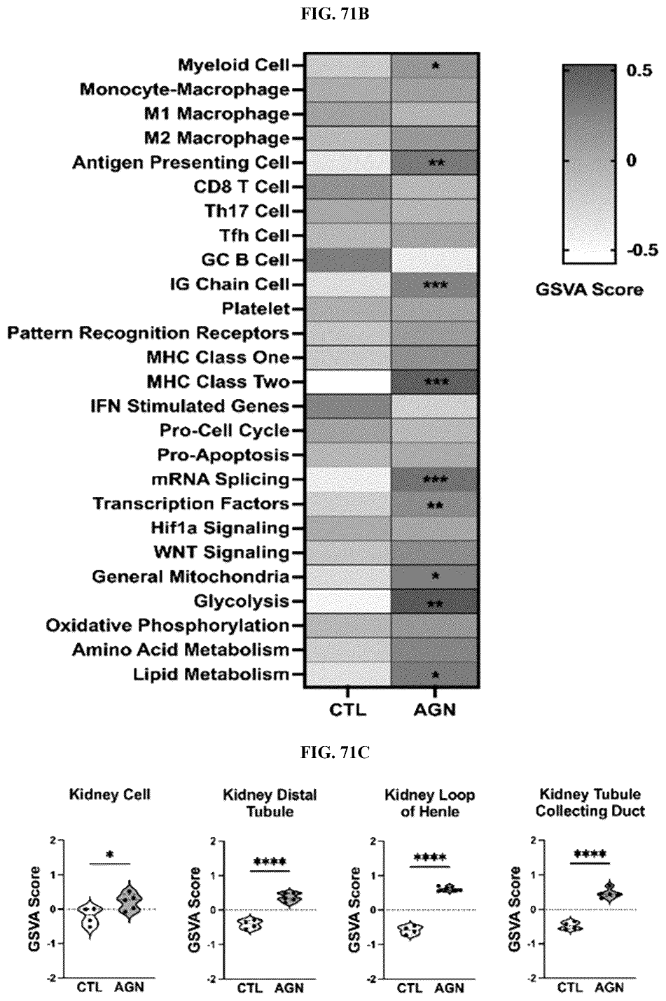

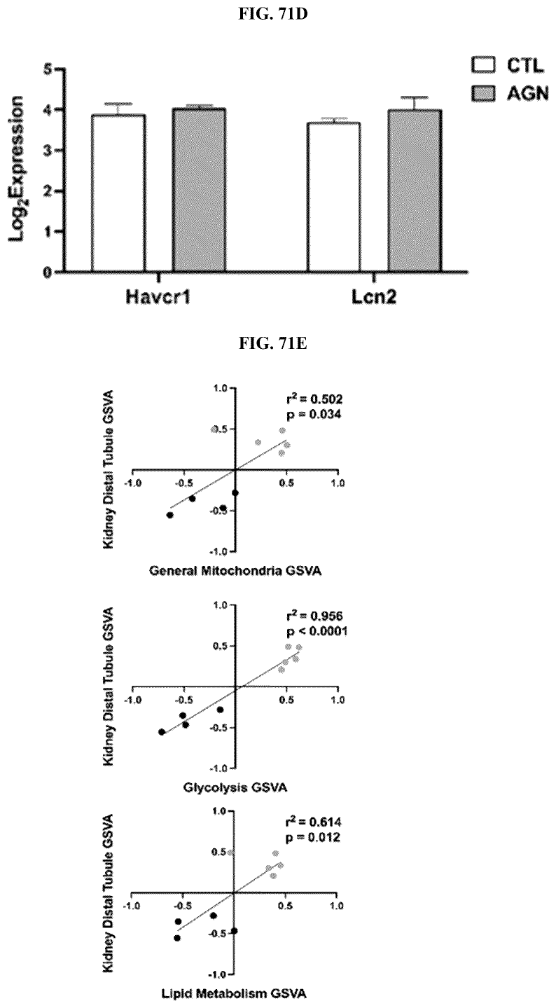

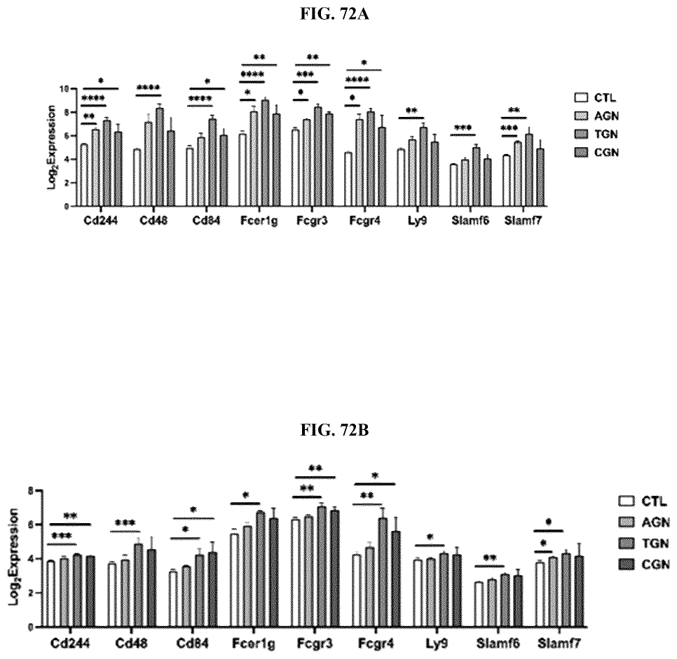

NZM2328.R27 mice exhibit resistance to kidney tubule damage. To examine the status of inflammation in the TI of R27 AGN mice relative to NZM2328 mice with acute disease, we compared overlap of DEGs from each cohort with gene signatures of immune cells and inflammatory processes (FIG. 71A). DEGs from the TI of NZM2328 AGN mice were significantly associated with signatures of APCs, myeloid cells, Mϕs, and MHC class II, indicating the presence of some immune infiltrates but not to the same extent as the inflammation in the glomeruli at this early point in disease (Table 21). In contrast, DEGs from R27 AGN mice only exhibited significant overlap with APC and MHC class II and were not associated with other inflammatory signatures (Table 22). Thus, differential expression analysis from R27 AGN mice indicated a lack of disease progression from the glomeruli to the kidney tubules, manifested by decreased inflammatory signatures in the TI.

GSVA analysis of the TI of R27 AGN mice confirmed the enrichment of APCs, IG chains, and myeloid cell signatures, but not Mϕs, indicating a less extensive infiltration of immune/inflammatory cells in the R27 mice than that in NZM mice. (FIG. 71B). Previous studies using the R27 strain found evidence that resistance to end organ damage might contribute to their decreased development of CGN (16). In support of this, we found that kidney tissue cell signatures and, in particular, kidney tubule cells were significantly increased in R27 AGN mice (FIG. 71C), whereas the kidney damage-associated genes, Havcr1 and Lcn2, were unchanged (FIG. 71D). We also found that gene signatures related to mitochondria, glycolysis, and lipid metabolism were increased (FIG. 71B) and were significantly correlated with the kidney tubule cell gene signature (FIG. 71E), suggesting that robust mitochondrial function may contribute to the kidney tubule cell enrichment observed in R27 AGN mice.

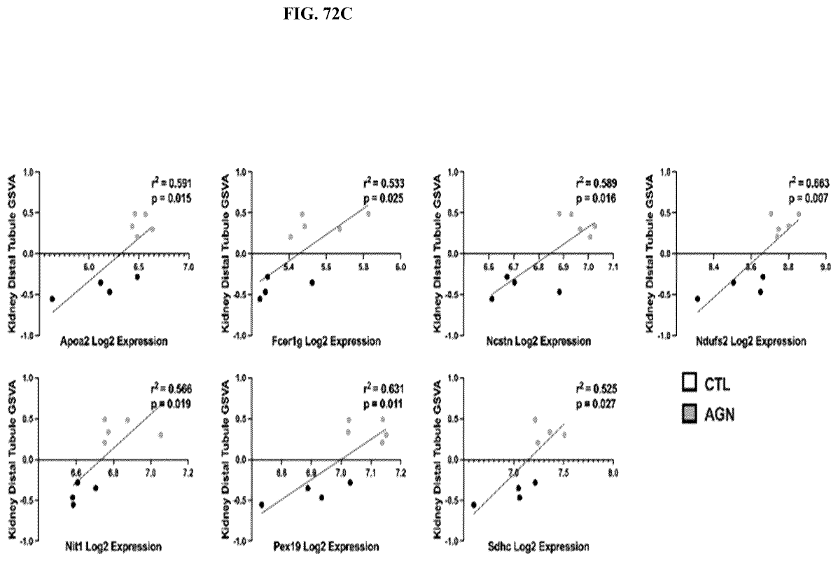

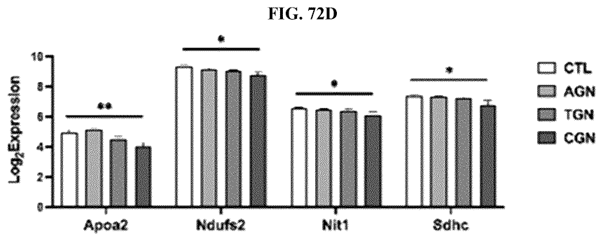

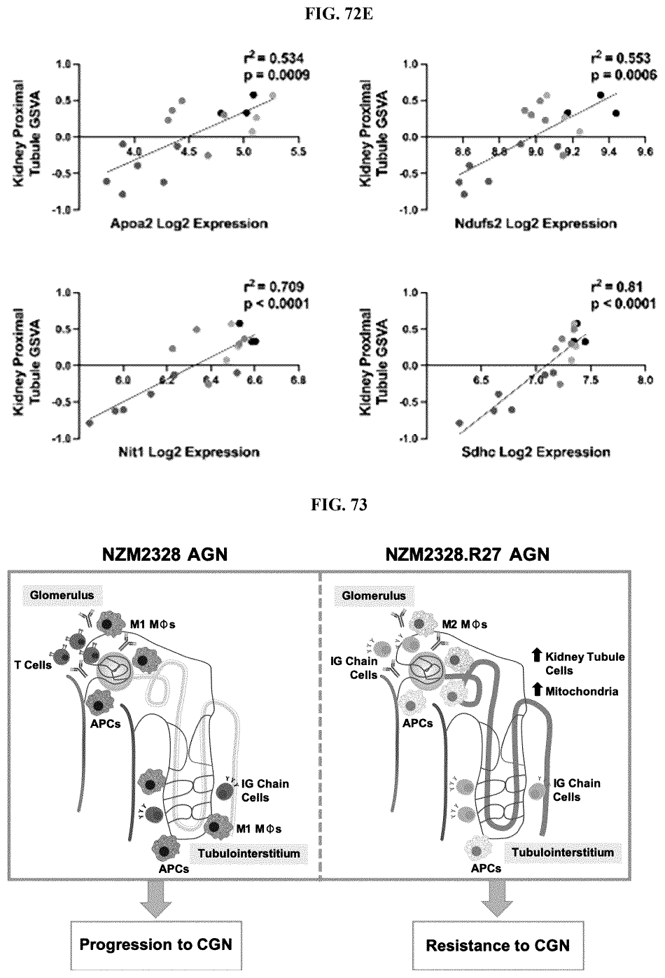

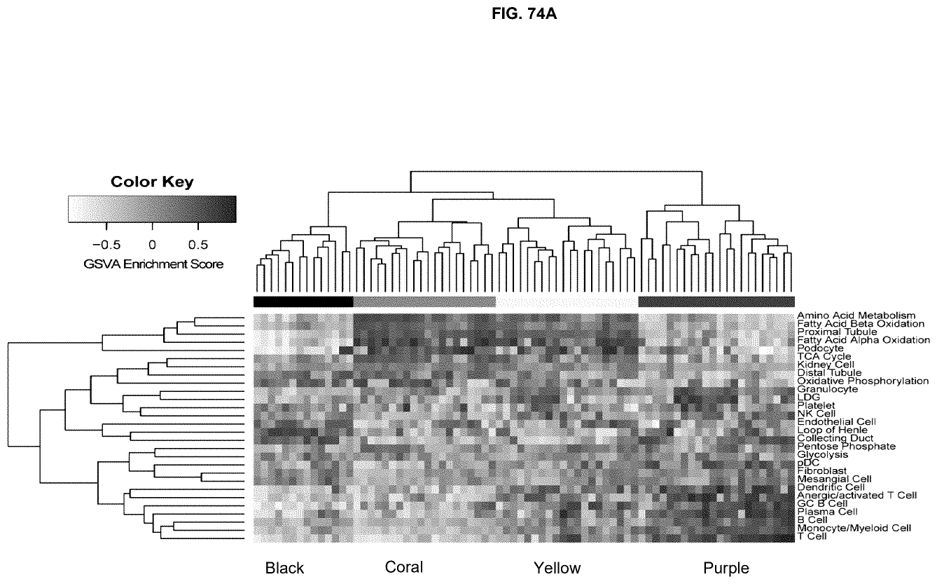

Kidney cell signatures enriched in NZM2328.R27 mice correlate with expression of chronic GN risk locus genes. The risk for progression to CGN in NZM2328 mice was associated with a 1.34 Mb region of chromosome 1 (Cgnz1) containing 45 genes (Table 20) (16). We analyzed differential expression of these CGN susceptibility genes in the glomerulus and TI of female NZM2328 AGN/TGN/CGN, and R27 AGN mice as compared to normal controls to determine their contribution to renal disease progression (FIGS. 72A-E). In the glomerulus, we found that genes encoding receptors expressed on immune cells and associated with inflammation, including Cd244, Fcer1g, Fcgr3, Fcgr4, and Slamf7 were significantly increased in NZM2328 AGN, whereas none was overexpressed in R27 kidneys, suggesting their expression is associated with increased inflammation and disease progression (FIG. 72A). Expression of these genes as well as additional immune-associated Cgnz1 locus genes was further increased at the height of inflammatory cell and pathway gene signature enrichment at the TGN stage and either maintained or decreased at the CGN stage in the glomerulus of NZM2328 mice.

In the kidney TI, there were no significant differences in expression of Cgnz1 locus genes in NZM2328 or R27 female AGN mice as compared to normal controls (FIG. 83). This result was reflective of the lack of pro-inflammatory signature enrichment in acute stage mice. However, at the TGN stage, expression of immune-associated Cgnz1 locus genes increased significantly over normal control mice, providing further evidence for the critical role of the CGN risk locus in progression to chronic disease (FIG. 72B).

The increased enrichment of kidney tubule cell signatures in R27 mice suggested that the genetic background of these mice, in which the Cgnz1 chronic risk locus was replaced with that from the non-lupus prone C57BL/J strain, rendered R27 kidneys resistant to tubule damage and prevented the progression from acute to chronic disease. To investigate the relationship between CGN risk locus gene expression and kidney tubule cell enrichment in R27 female AGN mice, we carried out linear regression analysis (FIG. 72C), and found that log 2 expression values for 7 of the 45 genes composing the Cgnz1 locus (Apoa2, Fcerig, Ncstn, Ndufs2, Nit1, Pex19, Sdhc) were significantly correlated with GSVA scores for kidney distal tubule cells and thus could play a role in promoting resistance to kidney damage. Notably, we found that the proteins encoded by these genes were involved in mitochondrial respiration (Ndufs2, Sdhc), metabolite processing (Apoa2, Ncstn, Nit1, Pex19) and immune signaling (Fcerig), suggesting a relationship to the increased enrichment of mitochondrial signatures in R27 AGN mice. Furthermore, log 2 expression values for 4 of these genes (Apoa2, Ndufs2, Nit1, and Sdhc) were significantly correlated with kidney tubule cell GSVA scores and significantly decreased in the TI of NZM2328 CGN mice as compared to normal controls (FIGS. 72D&E). To delve further into the functional pathways involving these kidney cell-associated genes, we identified upstream regulators (UPRs) using Ingenuity Pathway Analysis (IPA)(24) (Table 18). Notable UPRs predicted to drive expression of the 7 CGN risk locus genes included Rb1, Rictor, Wnt3a, Ctnnb1, and Hif1a and thus reflected the involvement of cell growth regulation, WNT signaling, and stress response pathways in the kidneys of R27 AGN mice. These results suggest that the cellular functions associated with expression of some of the Cgnz1 risk locus genes in R27 mice contribute to robust mitochondrial function and promote resistance to kidney tissue damage in the context of acute nephritis. Table 20 lists the 45 Cgnz1 Locus Genes. Table 21 lists the DEGs (in glomeruli and TI) from AGN mice. Table 22 lists the DEGs (in glomeruli and TI) from R27 mice.

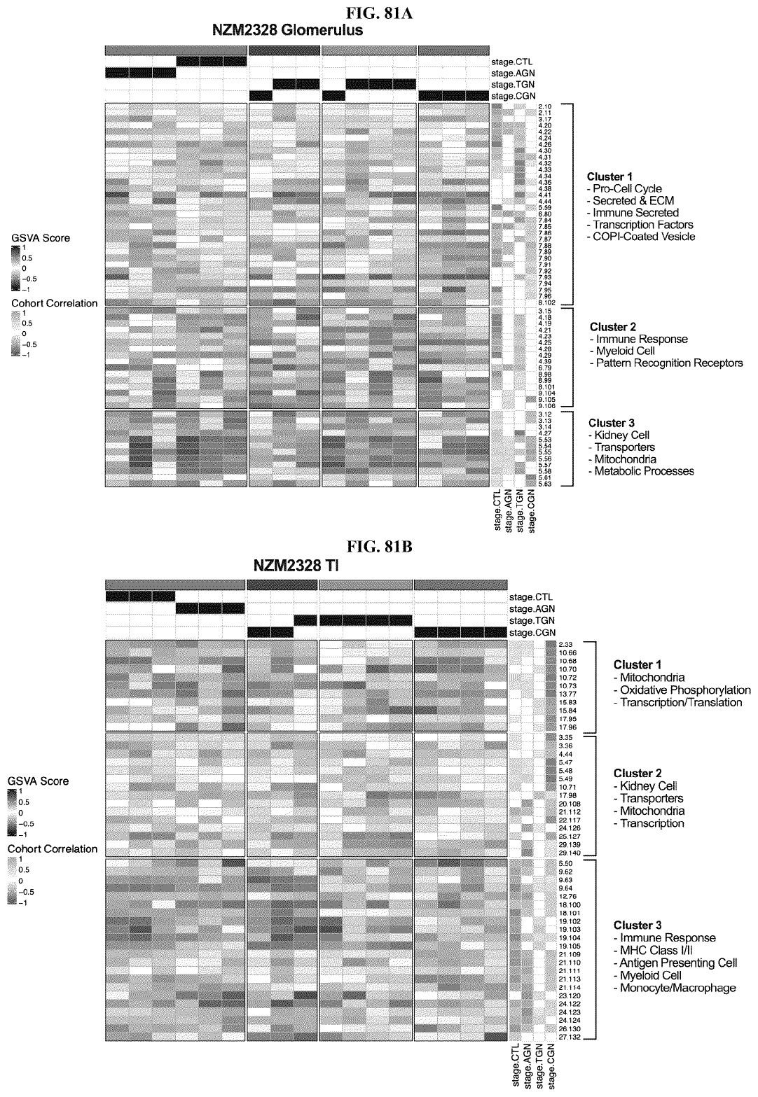

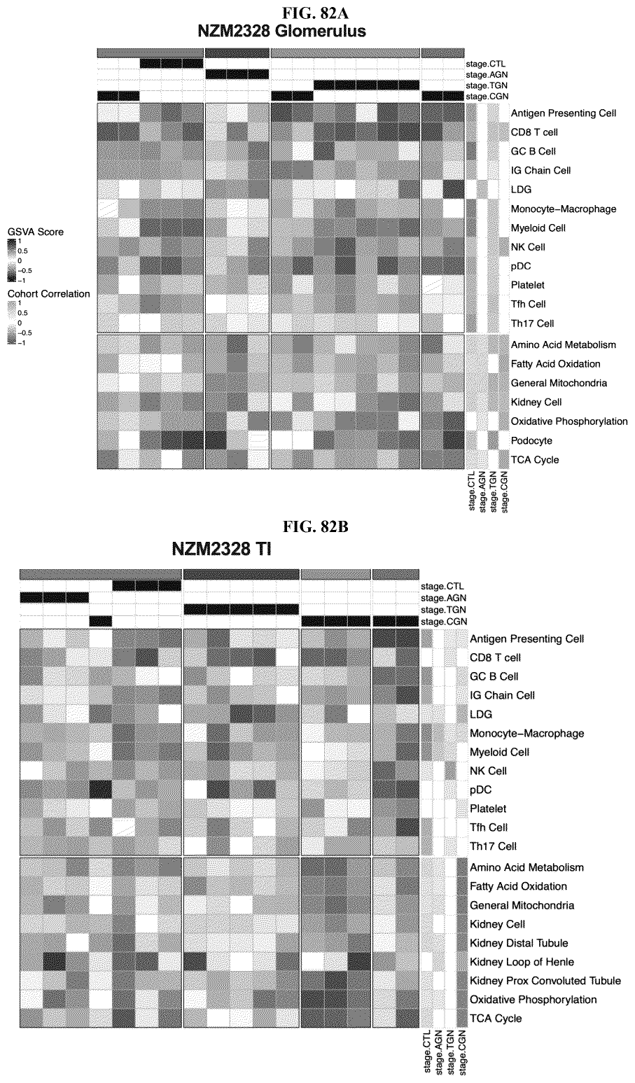

Gene co-expression network analysis identifies molecular profiles correlating with disease progression in NZM2328 mice. As an orthogonal approach to identify molecular patterns reflective of disease stage in NZM2328 mice in an unsupervised manner, we generated a network of co-expressed gene modules using multiscale embedded gene co-expression network analysis (MEGENA) and correlated individual gene modules with mouse GN stages (FIGS. 81A and B). MEGENA of gene expression results from NZM2328 mice generated 60 co-expressed gene modules for the glomerulus and 48 modules for the TI that were divided into three megaclusters and annotated based on gene overlap with curated gene signatures as well as gene ontology (GO) terms (Tables 26-1 to 26-60, and 27-1 to 27-48). Overall, the MEGENA-derived gene modules were representative of the major cell types and processes we had previously associated with GN using curated gene signatures, including inflammatory myeloid cells, kidney tissue cells, and metabolic processes. Furthermore, k-means clustering based on the MEGENA modules successfully separated mice into cohorts based on disease severity. In the glomerulus (FIG. 81A), the coral cluster (the left most cluster) of CTL and AGN mice was positively correlated with gene modules associated with kidney cells and metabolic processes, and negatively correlated with gene modules related to the immune/inflammatory response. Two clusters (maroon (2nd cluster from left) and green (3rd cluster from left)) contained a combination of TGN and CGN mice and were positively correlated with immune response modules and negatively correlated with kidney cell and metabolism modules. The final cluster of CGN mice (blue (the right most cluster)) had a negative correlation with immune response and kidney/metabolic modules but retained a high positive correlation with secreted immune factors. MEGENA results and correlations with disease stage in the TI (FIG. 81B) were similar to the glomerulus, but the resulting gene modules were more heavily skewed toward mitochondrial metabolism and the blue cluster of CGN mice was still positively correlated with the immune response-associated modules. In summary, this unsupervised approach employing co-expressed gene modules yielded results that closely resemble our previously identified molecular profiles of disease progression in NZM2328 mice.

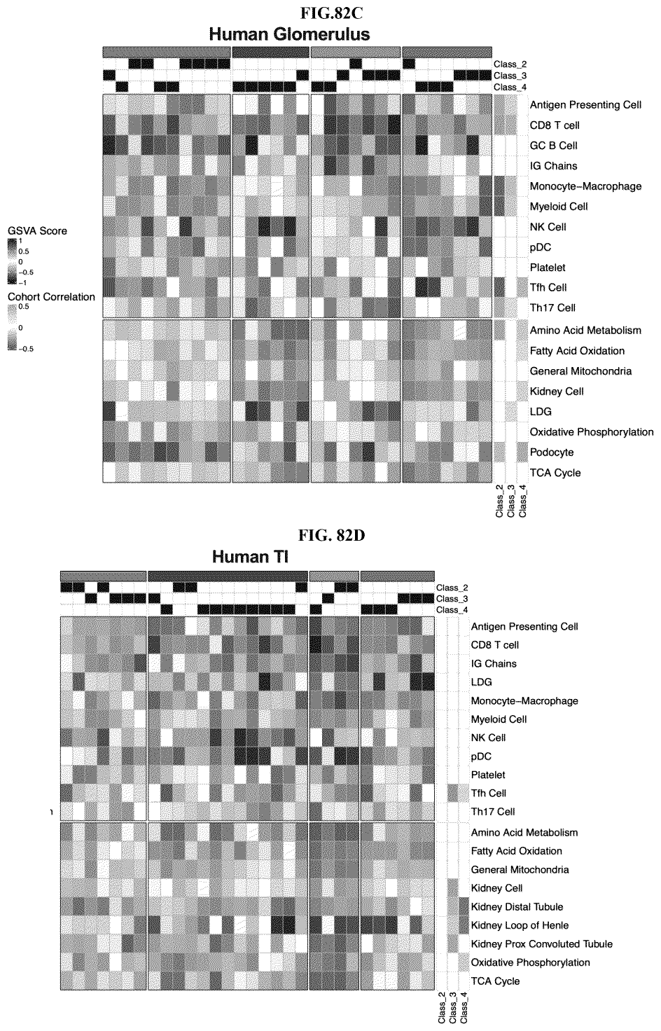

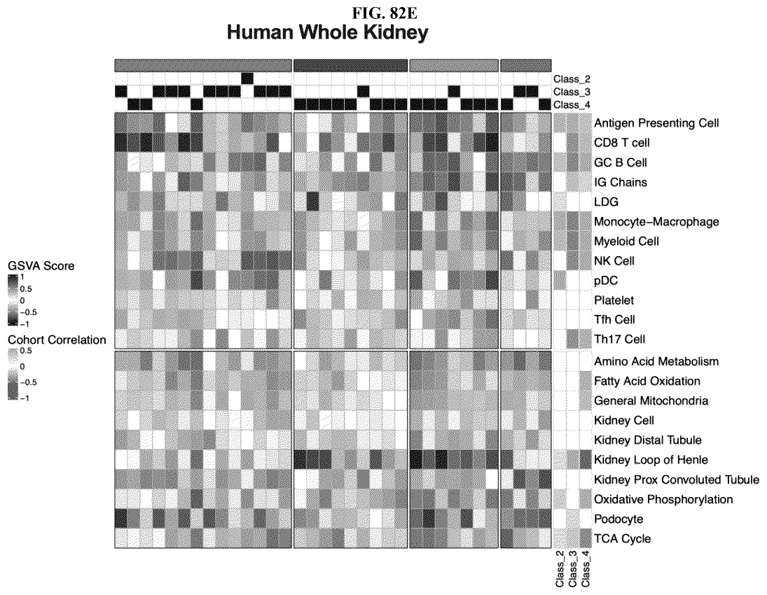

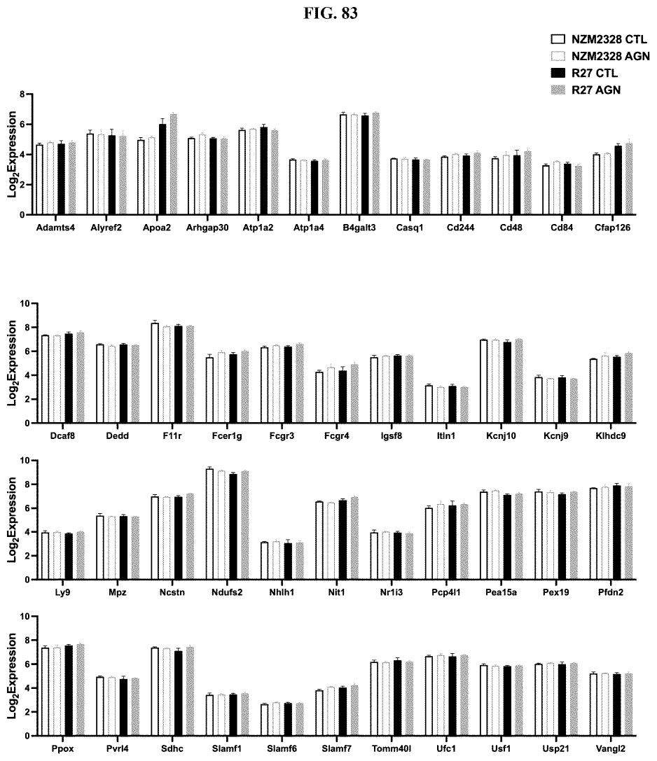

Identification of gene signatures characterizing GN stages in NZM2328 mice. We next sought to assemble a panel of curated gene signatures that would characterize the inflammatory environment in different stages of murine GN and determine whether similar immune profiles could be identified in human LN kidneys. To accomplish this, a core set of 22 GSVA gene signatures was selected based on significant enrichment in AGN, TGN, or CGN NZM2328 mice (Table 28-1 to 28-22, Table 2, FIGS. 67E and 68A). GSVA scores were then used as input for k-means clustering to form 4 clusters of mice from the glomerulus and TI gene expression datasets (FIG. 82A&B). In the glomerulus, AGN mice in the maroon cluster were characterized by slightly increased enrichment of inflammatory immune cells compared to CTL mice but retained enrichment of kidney tissue cell and metabolism gene signatures (FIG. 82A). TGN mice in the green cluster exhibited the highest enrichment of all inflammatory gene signatures accompanied by a decrease in metabolic and kidney cell signatures. CGN mice were divided among multiple clusters and thus reflected heterogeneity in immune profiles among mice at this stage of disease. Two CGN mice were placed in the coral cluster (the left most cluster) with CTL mice reflecting waning inflammation and retention of metabolic and kidney cell signatures. Another group of CGN mice with continued evidence of inflammatory gene signatures were found in the green cluster (the 3rd cluster from the left) with TGN mice. Finally, the blue cluster (the right most cluster) of CGN mice exhibited a relative de-enrichment of immune cells, kidney cells, and metabolic pathways indicative of a post-inflammatory state with evidence of end organ damage.

Gene expression-based clustering of the TI yielded similar results as the glomerulus with increasing inflammation and decreasing metabolism and kidney tubule cell gene signatures marking progression in disease severity (FIG. 82B). However, in the TI, the AGN mice clustered with CTLs and more of the CGN mice appeared to retain immune cell enrichment, reflecting persistent immune cell infiltration.

### Validation of NZM2328 Gene Expression Patterns in an Unrelated Dataset

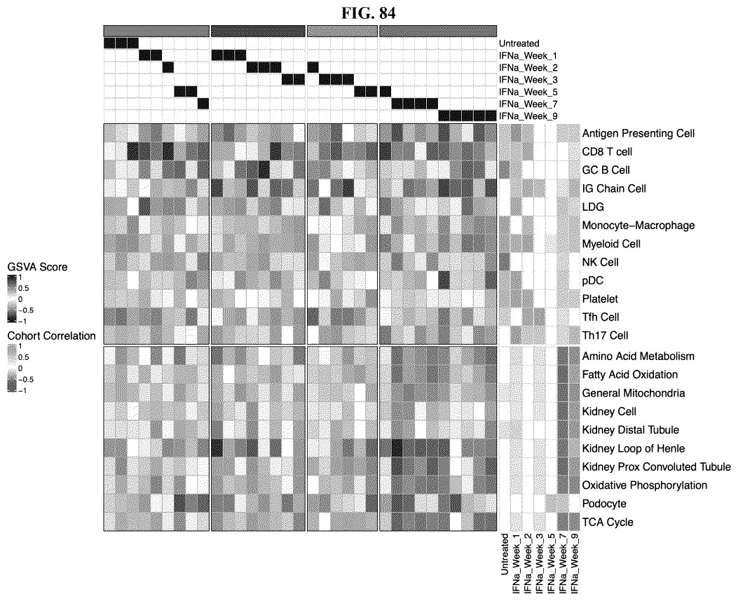

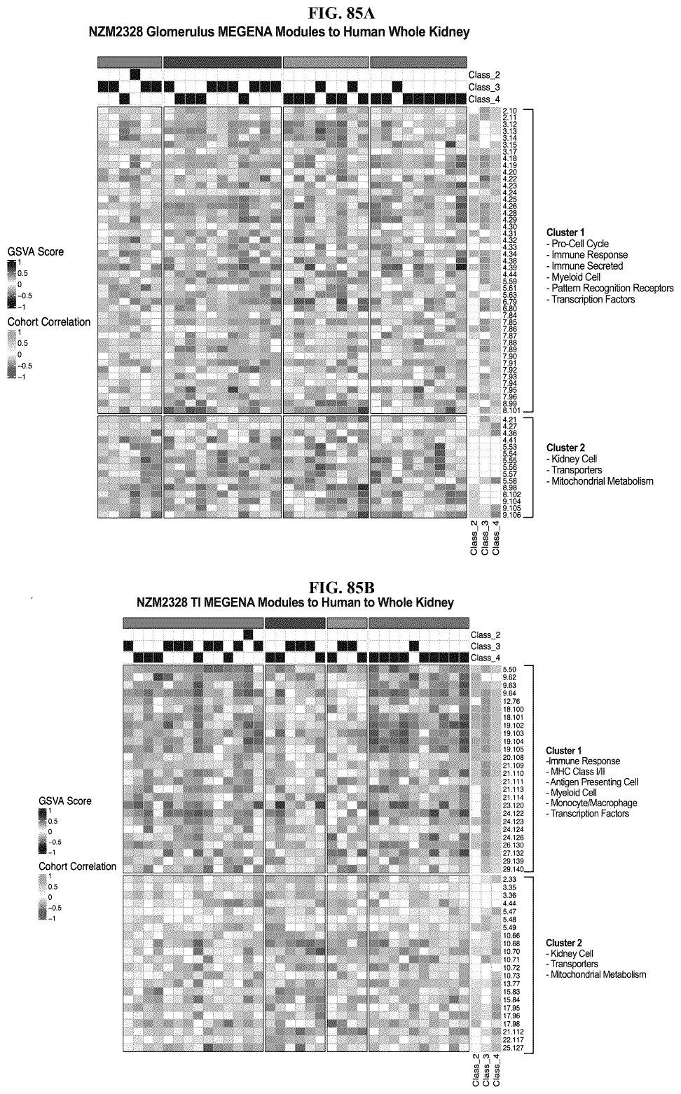

To validate the findings in NZM2328 mice in another lupus-prone strain, we applied the same approach to analysis of publicly available gene expression data from whole kidney tissue of the IFNα-accelerated NZB/W model (IFNα-NZB, GSE86423). Notably, the 22 curated gene expression signatures used to separate disease stages in NZM2328 mice, followed a similar enrichment pattern over a 9-week time course in IFNα-NZB mice indicating that this result was not unique to the gene expression dataset we generated from the NZM2328 strain (FIG. 84).

### Gene Signatures Characterizing GN Stages in NZM2328 Mice Identify Analogous Subsets of Human LN Patients

To determine whether immune profiles of NZM2328 mice with different stages of GN would translate to human lupus patients, we analyzed a publicly available gene expression dataset of microdissected glomeruli and TI from kidneys of patients with International Society of Nephrology (ISN) class II-IV LN as determined by histological classification (GSE32591) (27). We carried out GSVA using human orthologs of the 22 curated mouse gene signatures and identified 4 molecular endotypes by k-means clustering based on the pattern of enriched gene signatures in each individual patient (Table 17, FIGS. 82C-E). GSVA results of glomeruli and TI from kidneys of LN patients formed 4 patient clusters that exhibited similar gene set enrichment profiles to the nephritic kidneys of NZM2328 mice (FIGS. 82C&D). In both the glomerulus and TI, we observed a clear progression with increased enrichment of inflammatory cells corresponding with de-enrichment of kidney tissue cells as well as metabolic pathway signatures. In addition, the cohort correlations between LN classification and gene signature enrichment revealed increased correlations with pro-inflammatory cells in proliferative nephritis, particularly in the glomerulus, and corresponding negative correlations with kidney tissue cell signatures. However, whereas in the mouse kidneys we observed a post-inflammatory cluster of CGN stage mice, human LN samples with the greatest de-enrichment in metabolic and kidney cell signatures also retained a relatively high enrichment of immune/inflammatory cell signatures.

To confirm these results, we generated and analyzed a second gene expression dataset from whole kidneys of human LN patients in a similar manner (FIG. 82E). Notably, gene signature enrichment profiles of each human whole kidney subset more closely resembled clusters from mouse GN, including a cluster of samples that exhibited both de-enrichment of inflammatory signatures and metabolic signatures. GSVA of human whole kidney gene expression using the unsupervised MEGENA modules generated for NZM2328 mouse kidneys (FIGS. 81A and B) also yielded similar patterns of gene expression enrichment across LN patient clusters (FIGS. 85A and B). As an additional approach to establish similarities between mouse and human kidney gene expression profiles, we carried out MEGENA using the human LN whole kidney dataset. Then, MEGENA modules generated for the NZM2328 mouse (FIGS. 81A and B) were used as a reference to determine the preservation of gene module assignment between the mouse and human kidney gene co-expression networks (Tables 26-1 to 26-60, and 27-1 to 27-48). The results indicated that 22 MEGENA modules from the mouse glomerulus and 31 MEGENA modules from the mouse TI had a significant module preservation score (z-score>2) with human kidney modules indicating a high degree of overlap in their gene expression profiles. Overall, these results demonstrate that gene expression analysis can be used to classify stages of GN in lupus-prone mice and that mouse kidney endotypes can be translated to human LN patients.

FIGS. 85A-B show NZM2328 mouse MEGENA module-based clustering of human LN kidneys. K-means clustering (k=4) of whole kidney samples from human LN patients based on GSVA scores from human orthologs of the MEGENA modules from NZM2328 mouse microdissected glomeruli (A) and TI (B) used. The optimal number of module clusters was defined by the silhouette method and annotated by gene overlap with curated immunologic signatures and GO terms. Heatmap visualizations depict positive to negative GSVA scores on a red to blue gradient and positive to negative correlations between GSVA scores and disease classification on a gold to blue gradient.

### Discussion

The challenge of classifying disease pathology in heterogeneous presentations of LN has highlighted the need for a better understanding of the progression of disease in the kidneys of lupus patients and the risk factors for ESRD. To begin to address this, we utilized gene expression analysis to characterize stages of autoimmune inflammation leading up to the development of chronic disease in an established murine model of LN. Mice were classified in disease stages by histological comparison, matching mice by level of disease pathology and amount of IC deposition. This analysis revealed distinct immune profiles for acute disease, after initial IC deposition in the kidney glomerulus, transitional disease in which inflammatory cell and pathway enrichment is at its peak, and chronic disease in which the accumulated insults result in irreversible damage to the kidney tissue.

We found evidence of selective immune cell infiltration in the glomeruli of AGN mice, including enrichment for Monocyte/Mϕ, APC, MHC Class II, and Tfh cell gene signatures. This result reflects a limited set of immune/inflammatory cells present in the tissues at the initiation of AGN and likely reflects the cellular response to IC deposition. (1, 15, 25). Enrichment of apoptosis and PRR gene signatures at the AGN stage may reflect the widening innate immune response triggered by early DAMP release from the kidney tissue. At the AGN stage the glomeruli of NZM2328 mice were also enriched for CD8 T cells, which studies have found to be elevated in both human and mouse GN and have been linked with disease severity (26-28). However, it is possible that in the context of AGN, these CD8 T cells act in a more regulatory rather than effector cell capacity, as previously suggested (29).

Our classification of the progression of GN in NZM2328 mice uncovered a newly recognized, intermediate stage, between acute and chronic disease during which we observed the greatest level of immune activity. As GN in NZM2328 mice progressed to the transitional stage, we observed a striking increase in innate immune response pathways and evidence of significant myeloid cell infiltration in the kidney tissue. Previous molecular studies of lupus nephritis report a robust IFN response as the key feature distinguishing kidneys of lupus patients from healthy individuals and the TGN stage is when we first observed significant enrichment of an IFN signature in the glomeruli of diseased mice (30-32). In line with this result, we also found significant enrichment of M$ populations in TGN mice and, in particular, those with a pro-inflammatory, M1 rather than an alternatively activated, M2 gene signature. Mϕs with both an M1 and an M2 phenotype have been described in mouse models of lupus nephritis and associated with disease pathogenesis (33-36). However, despite the production of anti-inflammatory molecules by M2 Mϕs, the amplification of inflammatory cytokine production by immune and kidney tissue cells was found to overwhelm any regulatory response and promote disease progression. Kidney-infiltrating Mϕs are also important mediators of damage to the kidney tissue and we found that increases in Mϕ signatures in TGN mice were accompanied by decreases in kidney cell signatures and, in particular, podocytes. Podocytes are frequent targets of immune infiltration in the glomerulus and podocyte injury has been associated with proteinuria in lupus patients and is regarded as a precursor to end organ renal damage (37-39). There is a debate about whether podocyte injury is a primary or more down-stream event in the progression of LN. The current data indicate that in this murine model, detectable podocyte injury is down-stream of the initial events in AGN.

It has been reported that the low oxygen tension environment in the kidney becomes more hypoxic in lupus nephritis, correlates with disease severity, and is associated with mitochondrial dysfunction in lupus mouse models (40, 41). In addition, several studies supporting the “chronic hypoxia hypothesis” have identified hypoxia-induced damage in the TI as the final critical pathway leading to ESRD in human patients (3, 5, 42). Our results align with these studies as we observed enrichment of the hypoxia response pathway through Hifla in the glomeruli of TGN mice. Furthermore, heightened severity of disease pathology in CGN mice was accompanied by evidence of further damage to the kidney tissue, as well as a loss of mitochondrial and metabolic gene signatures suggestive of mitochondrial dysfunction. Therefore, our results support previous assertions that targeting the hypoxia response and mitochondrial dysfunction may be beneficial in the treatment of lupus patients (41, 43).

Glomeruli serve as the first connection points of kidney nephrons with the vasculature and thus, are also the initial targets of ICs and infiltrating immune cells before disease progresses downstream to the kidney tubules. As a result, damage to the kidney tubules is regarded as a diagnostic marker for progression to ESRD (7, 44). In line with this, enrichment of inflammatory cell and pathway gene signatures was delayed in the TI as compared to the glomeruli of nephritic mice and resulted in de-enrichment of kidney tubule cell gene signatures in the TI of CGN mice. We also found that the expression of kidney-damage associated genes Havcr1 and Lcn2(45-48) was significantly elevated in the TI of CGN mice. In addition, de-enrichment of metabolic gene signatures indicative of mitochondrial dysfunction was more prevalent in the TI of CGN mice suggesting that mitochondrial stress contributed to kidney tubule damage in late-stage disease.

Here, we also examined the mechanism(s) of resistance to chronic disease based on differences in gender and genetic background of lupus-prone NZM2328 mice. The increased prevalence of SLE in females over males in both human lupus patients and certain lupus mouse models implicates sex hormones in the pathogenesis of lupus nephritis (21, 23, 49, 50). Our analysis by both histology and gene expression-based approaches confirmed that male NZM2328 lupus-prone mice develop a milder form of AGN than female mice that does not progress to CGN (17). In addition, critical metabolic signatures, including glycolysis and oxidative phosphorylation, were decreased in male AGN mice suggestive of a dampened inflammatory response. Analysis of sex hormone-regulated gene signatures in the kidney did not indicate a difference in the estrogen response of female or male mice, which has been associated with lupus pathogenesis in both humans and mouse models (51-54). However, in many cases, the effects of estrogen regulation have been on immune cell populations and, therefore, we cannot discount an influence of estrogen regulation on circulating immune cells outside of the kidney tissue. In contrast to estrogens, androgens have been implicated in immunosuppression with decreased levels found in autoimmunity (55, 56). In line with this, androgen-regulated genes were de-enriched in male NZM2328 AGN mice and the genes contributing to this decrease were involved in cellular metabolism, suggesting a mechanism of androgen regulated immunosuppression through targeting metabolic pathways that is decreased in NZM nephritic mice.

We investigated the genetic-based resistance to chronic disease using female mice of the congenic strain, NZM2328.R27 (16). Interestingly, the glomeruli of R27 mice exhibited evidence of Mϕ infiltration, but the phenotype of these Mϕs was restricted to an anti-inflammatory, M2, phenotype and there was no enrichment of the pro-inflammatory, M1, gene signature observed in the base strain. This result suggests that the altered nature of the inflammatory response in R27 AGN mice contributes to end organ resistance to disease. Furthermore, the tubules of R27 AGN mice exhibited enrichment of gene signatures indicating a resistance to damaging pathologic processes stemming from inflamed glomeruli including increased kidney tubule cell signatures in conjunction with increased mitochondrial and metabolic gene signatures.

Since the R27 strain was derived by replacing the chronic disease risk locus, Cgnz1, of NZM2328, we examined the potential contribution of the 45 genes within this locus to resistance to CGN. We uncovered several pro-inflammatory genes with elevated expression in NZM2328 female mice, that would promote the activation of pathogenic immune populations such as M1 Mϕs and have been implicated in GN (57, 58). Furthermore, 7 risk locus genes that significantly correlated with kidney tubule cell signature enrichment in R27 AGN mice were involved in cell growth, metabolism, and WNT signaling. Involvement in boosting mitochondrial function could counteract the risk of mitochondrial stress and loss of function that were present in late-stage NZM2328 female mice. In addition, WNT signaling has been shown to have a positive role in resolving acute kidney injury, whereas it may promote maladaptive responses during chronic disease (59).

We have identified multiple mechanisms by which lupus-prone mice acquire resistance to chronic nephritis with implications for identifying risk factors for ESRD in human lupus patients. Interestingly, these mechanisms appear to be independent of the amount of IC deposition as all AGN mice (NZM2328 female, NZM2328 male, and R27 female) were matched by the level of pathology before monitoring disease progression. Resistance to chronic disease in male NZM2328 mice may have occurred at the initial point of IC deposition in the glomerulus, which failed to elicit a potent inflammatory response, possibly related to androgen-dependent suppression of energy-producing metabolic pathways. Resistance to chronic disease in R27 mice was associated with an altered composition of immune cells in the glomeruli that resulted in a lack of immune pathology downstream in the tubules. Moreover, the tubules in the R27 appear to be resistant to damage, as manifested by enhanced metabolic signatures. The resistance of tubules to damage related to immune activity in the glomerulus and/or hypoxia could play a pivotal role in preventing the typical inflammatory infiltrate in the TI of CGN, Thus, the absence of tubular dysfunction may have limited the inflammatory infiltrate in the TI and ultimately prevented additional damage to the kidney tissue.

Using a gene expression-based clustering approach, we have identified a core set of curated gene signatures able to classify disease stages of murine GN into molecular endotypes that effectively translate to human LN patients. Notably, human orthologs of the murine GN gene signatures identified a similar pattern in two independent cohorts of human LN patients consisting of increased enrichment of inflammatory cells and corresponding de-enrichment of metabolic pathways and kidney tissue cells associated with more advanced stages of kidney pathology. In current practice, the severity of LN pathogenesis is determined by histological classification, which is used to drive therapeutic decisions and assess the potential for terminal kidney damage (27,65,66). We found only modest correlation between ISN histological classification of renal pathology in human LN patients and molecular classification by gene expression profiling and the gene signature correlations that were identified were inconsistent across patients with the same ISN class and between datasets. This result emphasizes the subjectivity of histological assessment of renal pathology, and suggests that molecular classification may be a more robust and reproducible approach to classification of human LN.

An orthogonal, unsupervised approach to generate co-expressed gene modules (MEGENA) also identified similar molecular patterns that effectively classified mouse GN stages, human LN patients, and were highly conserved between species. This unsupervised approach supplies further validation for gene expressed-based profiles derived from curated gene signatures as well as the utility of lupus mice to recapitulate human LN at the molecular level. In summary, this work provides a comprehensive examination of the immune processes involved in progression of murine GN to chronic disease resulting in renal failure. In addition, this work presents a foundation for improved classification of LN based on molecular endotypes and illustrates the applicability of murine models to better understand the stages of human disease.

In summary, this, this work provides a comprehensive examination of the immune processes involved in progression of murine GN to chronic disease resulting in renal failure and provides new insights that could be applicable in understanding human LN.

### Methods

Mice. NZM2328 and NZM2328.R27 congenic mice were obtained/generated as previously described (14, 16). All mice were housed at the University of Virginia (UVA) Center of Comparative Medicine under pathogen-free conditions.

Histological characterization. Kidneys of NZM2328 and R27 mice were harvested and frozen sections were mounted on slides and the stage of GN was confirmed by histological classification as previously described (14, 16). Then laser capture microdissection (LCM) was used to isolate glomeruli and tubulointerstitial tissue from each kidney sample.

Gene expression analysis by microarray. RNA preparation and array hybridization were carried out by the UT Southwestern Microarray Core facility for NZM2328 and R27 female mice Affymetrix Clariom D Array of NZM2328 female and R27 mice and by the UVA Genome Analysis and Technology Core for the GeneChip Mouse 430 v2.0 array of NZM2328 female and male mice according to standard Affymetrix protocols.

Processing of raw microarray data was carried out with the R/Bioconductor package oligo. Affymetrix CEL files were background corrected and normalized using the robust multi-array average (RMA) method. Normalized data was transformed into log 2 intensity values and formatted as R expression set objects (E-sets). Principal component analysis (PCA) was used to inspect the datasets for outliers. E-sets were annotated using chip definition files (CDFs) corresponding to Affymetrix Clariom D (NZM2328 female and R27 mice) or Mouse 430 v2.0 (NZM2328 male mice) arrays. Low intensity probes were filtered by visual selection of thresholds at the dip in histograms of binned log 2-transformed probe intensities. Variance correction was carried out using the ebayes function in the R/Bioconductor LIMMA package. Resulting p-values were adjusted for multiple comparisons using the Benjamini-Hochberg correction that produced a false discovery rate (FDR) for each comparison. Probes were distilled down to differentially expressed (DE) probes with FDR<0.2 which were considered statistically significant.

Gene set variation analysis (GSVA). The R/Bioconductor package GSVA (20) (v1.36.3) was used as a non-parametric, unsupervised method to estimate the variation in enrichment of pre-defined gene sets in microarray data from NZM2328 mice. The input for GSVA was a matrix of log 2 expression values for all samples and a collection of gene signatures for immune cell types and functional pathways. Genes with multiple Affymetrix identifiers were selected based on the highest interquartile range (IQR) and probes with IQR=0 were filtered out. GSVA enrichment scores were calculated using a Kolgomorov-Smirnoff (KS)-like random walk statistic and were normalized across all samples to values between −1 and +1 indicative of negative enrichment and positive enrichment, respectively.

GSVA gene set generation. Gene sets used as input for GSVA are listed in Tables 19-1 to 19-36. The genes are grouped according to cell type or pathway signature. For example, Table 19-1 lists myeloid cell signature genes. Cell type and pathway gene signatures were generated based on literature mining, Mouse Genome Informatics (MGI)(60) gene ontology (GO) terms, and immune cell-specific expression derived from the Immunological Genome Project Consortium (ImmGen)(61). The glycolysis, oxidative phosphorylation, amino acid metabolism, and fatty acid oxidation gene signatures have been previously described (62). The cell type gene signatures were derived from Mouse CellScan, a tool for identification of cellular origin from mouse gene expression datasets. The pathway gene signatures were derived from the Mouse Biologically Informed Gene Clustering (BIG-C) tool for categorization of biological functions in mouse gene expression datasets. The human ortholog gene Tables (19A-1 to 19A-36) show certain human orthologs corresponding to the genes in mouse gene Tables (Tables 19-1 to 19-36) as indicated by the names listed in the table headers.

Linear regression analysis. Linear regression analysis between GSVA enrichment scores and log 2 gene expression values was carried out using GraphPad Prism software (v9.3.1). The goodness of fit is displayed as the R² value. The p-value indicates the significance of the slope of the regression line.

Ingenuity pathway analysis (IPA). Molecules upstream of selected Cgnz1 locus genes were identified using IPA upstream regulator (UPR) analysis (Qiagen) (24). UPRs with an overlap p-value<0.01 were considered significant.

Multiscale embedded gene co-expression network analysis (MEGENA). The MEGENA R package (23) was used to generate gene co-expression networks for NZM2328 mouse glomerulus and TI by inputting the top 5,000 row variance genes from the respective gene expression matrices. A planar filtered network (PFN) was formed using a false discovery rate (FDR) of 0.2. MEGENA multi-scale clustering analysis (MCA) used the PFN to form lineages of gene modules which were assigned “lineage” names based on their descendance from the root MEGENA module. Modules were functionally annotated by overlapping their gene symbols with curated mouse-specific functional signatures, immune cell, and kidney tissue cell signatures as well as the top GO terms (24) exhibiting the greatest coverage for each module. Annotations of MEGENA modules were considered significant if there were at least 3 overlapping gene symbols between the module gene symbols and annotation signature gene symbols, and the Fisher's p-value statistic of the overlap was p<0.2. A module eigengene (ME) was calculated for each module equivalent to the first principal component of a module's gene expression. Intracorrelations of sample traits were calculated for brief inspection. MEs were correlated to all sample traits and correlations were zeroed out where the p-value of the correlation was >=0.2. All second generation (gen2) MEGENA modules were retained for ensuing analysis. A gene expression set from human whole kidney biopsies was subjected to MEGENA analysis in a similar manner. Gen2 MEGENA modules from NZM2328 glomerulus and TI were examined for preservation in the MEGENA human kidney modules by utilizing an algorithm that generates z.summ composite scores of 20 preservation metrics (25).

K-means clustering. GSVA enrichment scores of gen2 MEGENA modules (Table 26-1 to 26-60, and Tables 27-1 to 27-48) or 22 curated immune cell, kidney cell, and metabolic pathway gene signatures (Tables 28-1 to 28-22) were used as input for k-means clustering performed with 1000 iterations to identify the most stable clusters for each dataset. Clustering results were visualized using the R package ComplexHeatmap (v 2.12) (26).

Human orthologs of mouse genes. In order to facilitate direct comparison of cell type and functional gene signature enrichment profiles between mouse and human gene expression datasets, mouse genes were translated into human orthologs. Orthologs are defined as genes in different species that evolved from a common ancestral gene and typically retain the same function. However, there are exceptions in that not all genes have direct mouse to human orthologs and not all predicted orthologs are “true” orthologs that retain the same function and expression pattern across species. To account for this, “true” human orthologs of the mouse gene sets were identified on a gene-by-gene basis using publicly available online databases (GeneCards, the Mouse Genome Informatics (MGI), and UniProtKB) as well as literature mining. Through this process, only genes with similar tissue expression, cellular localization, and functions between mouse and human were retained in the human gene sets.

Statistical analysis. P-values and odds ratios (ORs) for the overlap of DEGs with inflammatory cell types and pathways were calculated with a two-sided fisher's exact test in R with a confidence level of 0.95. All other statistical tests were carried out with GraphPad Prism (v9.3.1). Comparisons for two groups (CTL, AGN) were calculated using an unpaired, two-sided Welch's t-test. Comparisons for more than two groups (CTL, AGN, TGN, CGN) were calculated using Brown Forsythe and Welch's ANOVA followed by Dunnett's T3 multiple comparisons test.

TABLE 17
NZM2328 Kidney Histology, Listed by: Mouse Strain | Mouse ID |
Stage | Age | Protein urea | Glomerular histology |
Tubular histology | Interstitium histology | IgG deposition in Glom | C3 deposition in Glom | ANA

NZM2328-Female | NA1 | Normal | 8-9 weeks | None | | | | (−) | (−) | (−); NZM2328-Female |
NA2 | Normal | 8-9 weeks | None | | | | (−) | (−) | (−); NZM2328-Female | NA3 | Normal | 8-9
weeks | None | | | | (−) | (−) | (−); NZM2328-Female | NB1 | AGN | 36 weeks | 1+ | mesangial
expansion, immune cells infiltration | no tubular cell changes | mild immune cell infiltration in the
interstitium | (2+) | (2+) | (+); NZM2328-Female | NB2 | AGN | 36 weeks | 1+ | mesangial
expansion, immune cells infiltration | no tubular cell changes | mild immune cell infiltration in the
interstitium | (2+) | (2+) | (+); NZM2328-Female | NB3 | AGN | 36 weeks | 1+ | mesangial
expansion, immune cells infiltration | no tubular cell changes | mild immune cell infiltration in the
interstitium | (2+) | (2+) | (+); NZM2328-Female | NC1 | TGN | 36 weeks | 2+ | mesangial
expansion, immune cells infiltration | <15% tubular cells have tubular cell dialation, atrophy, casts
| moderate immune cell infiltration | (2~3+) | (2~3+) | (1+); NZM2328-Female | NC2 | TGN | 36
weeks | 2+ | mesangial expansion, immune cells infiltration | <15% tubular cells have tubular cell
dialation, atrophy, casts | moderate immune cell infiltration | (2~3+) | (2~3+) | (1+); NZM2328-
Female | NC3 | TGN | 36 weeks | 2+ | mesangial expansion, immune cells infiltration | <15%
tubular cells have tubular cell dialation, atrophy, casts | moderate immune cell infiltration | (2~3+)
| (2~3+) | (1+); NZM2328-Female | NC4 | TGN | 37 weeks | 2+ | mesangial expansion, immune
cells infiltration | <15% tubular cells have tubular cell dialation, atrophy, casts | moderate immune
cell infiltration | (2~3+) | (2~3+) | (1+); NZM2328-Female | NC5 | TGN | 37 weeks | 2+ |
mesangial expansion, immune cells infiltration | <15% tubular cells have tubular cell dialation,
atrophy, casts | moderate immune cell infiltration | (2~3+) | (2~3+) | (1+); NZM2328-Female
ND1 | CGN | 36 weeks | 3+ | mesangial expansion, immune cells infiltration, focal sclerosis |
| >80% tubular cells have tubular cell dialation, atrophy, casts | severe immune cell infiltration |
(3+) | (3+) | (2+); NZM2328-Female | ND2 | CGN | 36 weeks | 3+ | mesangial expansion, immune
cells infiltration, focal sclerosis | >80% tubular cells have tubular cell dialation, atrophy, casts |
severe immune cell infiltration | (3+) | (3+) | (2+); NZM2328-Female | ND3 | CGN | 39 weeks |
3+ | mesangial expansion, immune cells infiltration, focal sclerosis | >80% tubular cells have
tubular cell dialation, atrophy, casts | severe immune cell infiltration | (3+) | (3+) | (2+);
NZM2328-Female | ND4 | CGN | 39 weeks | 3+ | mesangial expansion, immune cells infiltration,
focal sclerosis | >80% tubular cells have tubular cell dialation, atrophy, casts | severe immune cell
infiltration | (3+) | (3+) | (2+); NZM2328-Female | ND5 | CGN | 39 weeks | 3+ | mesangial
expansion, immune cells infiltration, focal sclerosis | >80% tubular cells have tubular cell
dialation, atrophy, casts | severe immune cell infiltration | (3+) | (3+) | (2+); NZM2328-Female |
ND6 | CGN | 39 weeks | 3+ | mesangial expansion, immune cells infiltration, focal sclerosis
| >80% tubular cells have tubular cell dialation, atrophy, casts | severe immune cell infiltration |
(3+) | (3+) | (2+); NZM2328-Male | MA1 | Normal | 8-9 weeks | NONE | | | | (−) | (−) | (−);
NZM2328-Male | MA2 | Normal | 8-9 weeks | NONE | | | | (−) | (−) | (−); NZM2328-Male | MA3
| Normal | 8-9 weeks | NONE | | | | (−) | (−) | (−); NZM2328-Male | MB1 | AGN | 10 months | (+) |
mesangial expansion, mild immune cells infiltration | <2% focal and mild tubular cells changes |
mild immune cell infiltration in the interstitium | (2+) | (2+) | (1+); NZM2328-Male | MB2 | AGN
| 10 months | (+) | mesangial expansion, mild immune cells infiltration | <2% focal and mild
tubular cells changes | mild immune cell infiltration in the interstitium | (2+) | (2+) | (1+);
NZM2328-Male | MB3 | AGN | 10 months | (+) | mesangial expansion, mild immune cells
infiltration | <2% focal and mild tubular cells changes | mild immune cell infiltration in the
interstitium | (2+) | (2+) | (1+); NZM2328-Male | MB4 | AGN | 10 months | (+) | mesangial
expansion, mild immune cells infiltration | <2% focal and mild tubular cells changes | mild
immune cell infiltration in the interstitium | (2+) | (2+) | (1+); NZM2328-Male | MB5 | AGN | 10
months | (+) | mesangial expansion, mild immune cells infiltration | <2% focal and mild tubular
cells changes | mild immune cell infiltration in the interstitium | (2+) | (2+) | (1+); NZM2328-
Male | MB6 | AGN | 10 months | (+) | mesangial expansion, mild immune cells infiltration | <2%
focal and mild tubular cells changes | mild immune cell infiltration in the interstitium | (2+) | (2+) |
(1+); R27-Female | RA1 | Normal | 8-9 weeks | None | | | | (−) | (−) | (−); R27-Female | RA2 |
Normal | 8-9 weeks | None | | | | (−) | (−) | (−); R27-Female | RA3 | Normal | 8-9 weeks | None | | |
| (−) | (−) | (−); R27-Female | RA4 | Normal | 8-9 weeks | None | | | | (−) | (−) | (−); R27-Female |
RB1 | AGN | 12 months | 1+ | mesangial expansion, dialated loops, few infiltrated cells in
Glomeruli | no tubular cell changes | mild immune cell infiltration in the interstitium | (2+) | (2+) |
(+); R27-Female | RB2 | AGN | 12 months | 1+ | mesangial expansion, dialated loops, few
infiltrated cells in Glomeruli | no tubular cell changes | mild immune cell infiltration in the
interstitium | (2+) | (2+) | (+); R27-Female | RB3 | AGN | 12 months | 1+ | mesangial expansion,
dialated loops, few infiltrated cells in Glomeruli | no tubular cell changes | mild immune cell
infiltration in the interstitium | (2+) | (2+) | (+); R27-Female | RB4 | AGN | 12 months | 1+ |
mesangial expansion, dialated loops, few infiltrated cells in Glomeruli | no tubular cell changes |
mild immune cell infiltration in the interstitium | (2+) | (2+) | (+); R27-Female | RB5 | AGN | 12
months | 1+ | mesangial expansion, dialated loops, few infiltrated cells in Glomeruli | no tubular
cell changes | mild immune cell infiltration in the interstitium | (2+) | (2+) | (+)

TABLE 18
IPA Upstream Regulator Analysis

| Upstream | | |
|---|---|---|
| Regulator | Regulator Annotation | Target Molecule(s) |
| KDM5A | Histone demethylase | NDUFS2, NIT1, SDHC |
| MED30 | Transcriptional coactivator | NDUFS2, SDHC |
| RB1 | Cell cycle regulator | NDUFS2, NIT1, SDHC |
| PITX2 | WNT signaling | NDUFS2, SDHC |
| WNT3A | WNT signaling | NDUFS2, SDHC |
| CTNNB1 | WNT signaling | FCER1G, NDUFS2, SDHC |
| NDRG1 | Stress response | APOA2 |
| HIF1A | Hypoxia | NDUFS2, SDHC |
| SLC4A1 | Kidney Ion Transport | FCER1G |
| CSK | Cell growth regulator | FCER1G |
| RICTOR | Cell growth regulator | NDUFS2, SDHC |
| COPS5 | Cell cycle regulator | FCER1G |
| APOA1 | Lipid transport | APOA2 |
| INSR | Cell growth regulator | APOA2 |
| Hmgn3 | Cell growth regulator | APOA2 |
| NCSTN | WNT signaling | FCER1G |
| CYLD | WNT signaling | FCER1G |
| FST | Cell growth regulator | APOA2 |
| PTPRJ | Cell growth regulator | FCER1G |

Tables 19-1 to 19-36: GSVA gene sets. In Tables 19-1 to 19-36, the genes are listed by: Gene symbol|Entrez ID.

TABLE 19-1
Myeloid Cell (also see Table 19A-25 for human orthologs)

Aif1 | 11629; Aoah | 27052; Arg1 | 11846; Batf2 | 74481; Bmx | 12169; Bpi | 329547; Cd300lf |
246746; Cd33 | 12489; Cd68 | 12514; Clec12a | 232413; Clec4a2 | 26888; Clec4b1 | 69810;
Clec4d | 17474; Clec4e | 56619; Clec4g | 75863; Clec4n | 56620; Clec5a | 23845; Clec7a | 56644;
Csf2ra | 12982; Csf2rb | 12983; Csf3r | 12986; Cst3 | 13010; Cxcl9 | 17329; Cyba | 13057; Cybb |
13058; Fcer1a | 14125; Fcgr1 | 14129; Fcgr3 | 14131; Fcgr4 | 246256; Ffar2 | 233079; Fgr | 14191;
Igsf6 | 80719; Il1rn | 16181; Lilrb4a | 14728; Lyz2 | 17105; Mpeg1 | 17476; Ms4a6d | 68774;
Nlrp12 | 378425; Nlrp3 | 216799; Olr1 | 108078; P2rx5 | 94045; Pilra | 231805; Pla2g2d | 18782;
S100a9 | 20202; Scarb1 | 20778; Serping1 | 12258; Slamf8 | 74748; Trem1 | 58217; Treml4 |
224840; Vsig4 | 278180

TABLE 19-2
Monocyte-Macrophage (also see Table 19A-23 for human orthologs)

Ace | 11421; Adam8 | 11501; Adgre1 | 13733; Apobr | 171504; Apoc1 | 11812; C1qa | 12259;
C1qb | 12260; C1qc | 12262; C2 | 12263; C4b | 12268; C6 | 12274; Ccl7 | 20306; Ccl8 | 20307;
Cd14 | 12475; Cd163 | 93671; Cd300e | 217306; Cd5l | 11801; Cfb | 14962; Cfd | 11537; Cfp |
18636; Csf1r | 12978; Cxcl13 | 55985; Folr2 | 14276; Ifi211 | 381308; Il31ra | 218624;
Lgals12 | 56072; Lyve1 | 114332; Marco | 17167; Megf10 | 70417; Mertk | 17289; Mfge8 |
17304; Mrc1 | 17533; Ms4a4a | 666907; Msr1 | 20288; Pla2g5 | 18784; Scarf2 | 224024;
Siglec1 | 20612

TABLE 19-3
M1 Macrophage (also see Table 19A-18 for human orthologs)

Aoah | 27052; Arhgap24 | 231532; Ccrl2 | 54199; Cd300lf | 246746; Cd38 | 12494; Cd99 |
673094; Cfb | 14962; Cp | 12870; Cpd | 12874; Cxcl10 | 15945; Ddx58 | 230073; Ddx60 |
234311; Ebi3 | 50498; F11r | 16456; Fam176b | 230752; Fam26f | 215900; Fpr1 | 14293; Fpr2
| 14289; Gbp6 | 100702; Gngt2 | 14710; Gpr18 | 110168; H2-Q6 | 110557; H2-T10 | 15024;
Herc6 | 67138; Hp | 15439; Ifi44 | 99899; Ifit2 | 15958; Irak3 | 73914; Isg15 | 100038882; Itgal
| 16408; Marco | 17167; Mpa2l | 100702; Ms4a4c | 64380; Mx1 | 17857; Nfkbiz | 80859;
Oasl1 | 231655; Pstpip2 | 19201; Pyhin1 | 236312; Rsad2 | 58185; Sepx1 | 27361; Slfn1 |
20555; Slfn4 | 20558; Slfn8 | 276950; Stat1 | 20846; Stat2 | 20847; Tlr2 | 24088; Tuba4a |
22145; Xaf1 | 327959; Zbp1 | 58203

TABLE 19-4
M2 Macrophage (also see Table 19A-19 for human orthologs)

Actn1 | 109711; Amz1 | 231842; Atp6v0a1 | 11975; Atp6v0d2 | 242341; Bcar3 | 29815;
Cd300ld | 217305; Cd83 | 12522; Chst7 | 60322; Clec10a | 17312; Clec7a | 56644; Egr2 |
13654; Emp2 | 13731; Flrt2 | 399588; Gnb4 | 14696; I16st | 16195; Lmna | 16905; Matk |
17179; Mmp12 | 17381; Mmp9 | 17395; Mrc1 | 17533; Myc | 17869; Olfm1 | 56177; P2ry1 |
18441; Plk2 | 20620; Ptgs1 | 19224; Ptpla | 30963; Rhoj | 80837; Socs6 | 54607; Tanc2 |
77097; Tcfec | 21426; Tiam1 | 21844; Tmem158 | 72309; Vwf | 22371

TABLE 19-5
Antigen Presenting Cell (also see Table 19A-3 for human orthologs)

Cd1d1 | 12479; Cd74 | 16149; Ciita | 12265; H2-Aa | 14960; H2-Ab1 | 14961; H2-DMa |
14998; H2-DMb2 | 15000; H2-Ea | 100504404; H2-Eb1 | 14969; H2-Oa | 15001; H2-Ob |
15002; Mgl2 | 216864; Rfx5 | 53970; Scimp | 327957

TABLE 19-6
CD8 T cell (also see Table 19A-4 for human orthologs)

Cd8a | 12525; Cd8b1 | 12526

TABLE 19-7
Th17 Cell (also see Table 19A-33 for human orthologs)

Ahr | 11622; Cd3d | 12500; Cd3e | 12501; Cd3g | 12502; Il17a | 16171;
Il17f | 257630; Il21 | 60505; Il22 | 50929; Irf4 | 16364; Nfxl1 | 100978;
Pgf | 18654; Rora | 19883; Rorc | 19885; Traf6 | 22034

TABLE 19-8
Tfh Cell (also see Table 19A-32 for human orthologs)

Ascl2 | 17173; Bcl6 | 12053; Cxcr5 | 12145; Icos | 54167; Nt5e | 23959;
Pdcd1 | 18566; Tnfrsf4 | 22163

TABLE 19-9
Treg (also see Table 19A-35 for human orthologs)

Foxp3 | 20371; Gpr83 | 14608; Ikzf2 | 22779; Ikzf4 | 22781; Lrrc32 |
434215; Penk | 18619; Tnfrsf4 | 22163; Tnfrsf9 | 21942

TABLE 19-10
GC B Cell (also see Table 19A-7 for human orthologs)

Fcrlb | 435653; Gcsam | 14525; Klh16 | 239743; Lrmp | 16970;
Mybl1 | 17864; Nuggc | 100503545; Rgs13 | 246709

TABLE 19-11
IG Chain Cell (also see Table 19A-11 for human orthologs)

Igha | 238447; Ighd | 380797; Ighe | 380792; Ighg1 | 16017; Ighg2a | 380793; Ighg2b | 16016;
Ighg2c | 404711; Ighg3 | 380795; Ighj2 | 777654; Ighj3 | 777655; Ighj4 | 777656; Ighm |
16019; Ighv1-12 | 629860; Ighv1-14 | 629863; Ighv1-15 | 629865; Ighv1-19 | 382692; Ighv1-
20 | 668497; Ighv1-22 | 780883; Ighv1-26 | 629884; Ighv1-31 | 629893; Ighv1-34 | 628614;
Ighv1-36 | 629897; Ighv1-37 | 668517; Ighv1-39 | 780891; Ighv1-4 | 432702; Ighv1-43 |
629908; Ighv1-47 | 629915; Ighv1-49 | 629925; Ighv1-5 | 668469; Ighv1-50 | 780929; Ighv1-
52 | 382696; Ighv1-59 | 432708; Ighv1-61 | 674018; Ighv1-62-1 | 668544; Ighv1-62-2 |
238448; Ighv1-63 | 780956; Ighv1-66 | 380824; Ighv1-7 | 668474; Ighv1-71 | 619886; Ighv1-
75 | 622728; Ighv1-76 | 100775174; Ighv1-77 | 619994; Ighv1-78 | 213570; Ighv1-80 |
668589; Ighv1-81 | 668591; Ighv1-82 | 100775175; Ighv1-85 | 668597; Ighv1-9 | 668478;
Ighv10-1 | 380808; Ighv10-3 | 380809; Ighv11-1 | 780804; Ighv11-2 | 780818; Ighv12-3 |
435321; Ighv13-2 | 629842; Ighv14-1 | 780801; Ighv14-3 | 238418; Ighv14-4 | 629826;
Ighv15-2 | 780864; Ighv2-2 | 777686; Ighv2-3 | 238412; Ighv2-4 | 668389; Ighv2-5 | 638605;
Ighv2-6 | 630486; Ighv2-6-8 | 791090; Ighv2-9 | 640046; Ighv2-9-1 | 791089; Ighv3-1 |
780803; Ighv3-4 | 629833; Ighv3-5 | 633457; Ighv3-6 | 780829; Ighv3-8 | 780831; Ighv4-1
780802; Ighv5-12-4 | 628098; Ighv5-9-1 | 641178; Ighv6-3 | 629845; Ighv6-6 | 238427; Ighv6-
7 | 432701; Ighv7-1 | 780799; Ighv7-3 | 629822; Ighv7-4 | 632654; Ighv8-11 | 636462; Ighv8-
12 | 780960; Ighv8-6 | 629930; Ighv9-1 | 195180; Ighv9-2 | 629818; Ighv9-3 | 780825; Ighv9-
4 | 636260; Igk | 243469; Igkc | 16071; Igkj1 | 110759; Igkj2 | 110760; Igkj4 | 110762; Igkj5 |
110763; Igkv1-110 | 381777; Igkv1-117 | 16098; Igkv1-122 | 434024; Igkv1-132 | 243423;
Igkv1-133 | 628027; Igkv1-135 | 243420; Igkv1-88 | 692154; Igkv1-99 | 434028; Igkv10-94 |
667550; Igkv10-95 | 434031; Igkv11-125 | 243428; Igkv12-38 | 620050; Igkv12-41 | 619960;
Igkv12-44 | 545851; Igkv12-46 | 692245; Igkv12-89 | 384411; Igkv12-98 | 435900; Igkv13-84
692152; Igkv13-85 | 434036; Igkv14-100 | 243439; Igkv14-111 | 545847; Igkv14-130 |
628072; Igkv16-104 | 381778; Igkv17-121 | 667435; Igkv17-127 | 243433; Igkv18-36 |
620088; Igkv19-93 | 692161; Igkv2-109 | 628268; Igkv2-112 | 381776; Igkv20-101-2 |
724035; Igkv3-1 | 108004; Igkv3-10 | 667924; Igkv3-12 | 667914; Igkv3-2 | 626583; Igkv3-3 |
667946; Igkv3-4 | 626347; Igkv3-5 | 667940; Igkv3-7 | 108005; Igkv3-9 | 667928; Igkv4-50 |
381782; Igkv4-51 | 619806; Igkv4-53 | 546213; Igkv4-55 | 385253; Igkv4-57 | 235952; Igkv4-
57-1 | 384514; Igkv4-58 | 381831; Igkv4-59 | 667683; Igkv4-61 | 546244; Igkv4-63 | 628571;
Igkv4-68 | 384515; Igkv4-69 | 628541; Igkv4-70 | 385120; Igkv4-71 | 434586; Igkv4-72 |
385109; Igkv4-73 | 628516; Igkv4-74 | 236047; Igkv4-78 | 545849; Igkv4-79 | 213684; Igkv4-
80 | 545848; Igkv4-81 | 243453; Igkv4-86 | 243451; Igkv4-90 | 434034; Igkv4-91 | 434033;
Igkv4-92 | 384410; Igkv5-37 | 384417; Igkv5-39 | 620017; Igkv5-43 | 381783; Igkv5-45 |
545850; Igkv5-48 | 619846; Igkv6-14 | 667881; Igkv6-15 | 108022; Igkv6-17 | 667865; Igkv6-
20 | 108024; Igkv6-23 | 637227; Igkv6-25 | 381784; Igkv6-29 | 620191; Igkv6-32 | 434039;
Igkv7-33 | 243461; Igkv8-16 | 640340; Igkv8-19 | 232065; Igkv8-21 | 620400; Igkv8-24 |
677858; Igkv8-27 | 434041; Igkv8-28 | 434040; Igkv8-30 | 384419; Igkv8-34 | 620126; Igkv9-
120 | 434025; Igkv9-123 | 628144; Igkv9-124 | 243431; Iglc1 | 110785; Iglc2 | 110786; Iglc3 |
110787; Iglj1 | 404737; Iglj2 | 404739; Iglj3 | 404740; Iglv1 | 16142; Iglv2 | 110612; Iglv3 |
404743

TABLE 19-12
Platelet (also see Table 19A-28 for human orthologs)

Cd9 | 12527; Cxcl5 | 20311; Esam | 69524; Gp1ba | 14723; Gp5 |
14729; Gp6 | 243816; Gp9 | 54368; Ly6g6d | 114654; Mmrn1 | 70945;
Myl9 | 98932; Pear1 | 73182; Pf4 | 56744; Plek | 56193; Ppbp |
57349; Slc35d3 | 76157; Treml1 | 71326; Tspan9 | 109246

TABLE 19-13
Pattern Recognition Receptors (also see Table 19A-27 for human orthologs)

Ager | 11596; Alpk1 | 71481; Apobec3 | 80287; Cd209a | 170786; Cd209e | 170780; Clec4d |
17474; Clec4e | 56619; Clec7a | 56644; Ddx58 | 230073; Ifi209 | 236312; Irak4 | 266632; Irf7 |
54123; Irf8 | 15900; Irf9 | 16391; Lrrfip1 | 16978; Marco | 17167; Mefv | 54483; Nlrc3 |
268857; Nlrc5 | 434341; Nlrp10 | 244202; Nlrp12 | 378425; Nlrp3 | 216799; Nod2 | 257632;
Oas2 | 246728; Oas3 | 246727; Oasl2 | 23962; Pglyrp1 | 21946; Tlr1 | 21897; Tlr5 | 53791;
Tlr7 | 170743; Tlr9 | 81897; Unc93b1 | 54445

TABLE 19-14
MHC Class One (also see Table 19A-20 for human orthologs)

B2m | 12010; Cd1d1 | 12479; Erap1 | 80898; Fcgrt | 14132; H2-D1 | 14964; H2-D2 | 110536;
H2-D3 | 110553; H2-D4 | 14967; H2-K1 | 14972; H2-M1 | 224756; H2-M2 | 14990; H2-M3 |
14991; H2-M5 | 240095; H2-M9 | 14997; H2-Q1 | 15006; H2-Q10 | 15007; H2-Q2 | 15013;
H2-Q4 | 15015; H2-Q5 | 15016; H2-Q6 | 110557; H2-Q7 | 15018; H2-Q8 | 15019; H2-Q9 |
110558; H2-T10 | 15024; H2-T22 | 15039; H2-T23 | 15040; H2-T24 | 15042; H2-T3 | 15043;
H2-T9 | 15051; Lrmp | 16970; Lrmp | 16970; Mill2 | 243864; Mr1 | 15064; Tap1 | 21354;
Tap2 | 21355; Tapbp | 21356; Tapbpl | 213233

TABLE 19-15
MHC Class Two (also see Table 19A-21 for human orthologs)

Cd74 | 16149; Ciita | 12265; H2-Aa | 14960; H2-Ab1 | 14961;
H2-DMa | 14998; H2-DMb2 | 15000; H2-Eb1 | 14969; H2-eb2 |
381091; H2-Oa | 15001; H2-ob | 15002; Rfx5 | 53970

TABLE 19-16
IFN Stimulated Genes (also see Table
19A-10 for human orthologs)

Eif2ak2 | 19106; Gbp10 | 626578; Gbp2 | 14469; Gvin1 | 74558;
Ifi204 | 15951; Ifi207 | 226691; Ifi213 | 623121; Ifi30 | 65972;
Ifi44 | 99899; Ifitm1 | 68713; Iigp1 | 60440; Irgm1 | 15944;
Irgm2 | 54396; Mx1 | 17857; Parp9 | 80285; Rsad2 | 58185

TABLE 19-17
Pro-Cell-Cycle (also see Table 19A-31 for human orthologs)

Aurkb | 20877; Bub1 | 12235; Bub1b | 12236; Ccdc88a | 108686; Ccnd2 | 12444; Ccng1 |
12450; Cdc20 | 107995; Cdc25b | 12531; Cdca2 | 108912; Cdca5 | 67849; Cdk14 | 18647;
Cdk5 | 12568; Cdk6 | 12571; Cenpc1 | 12617; Cep55 | 74107; Clspn | 269582; Dbf4 | 27214;
Dsn1 | 66934; E2f2 | 242705; E2f3 | 13557; Ect2 | 13605; Esco2 | 71988; Espl1 | 105988;
Foxm1 | 14235; Incenp | 16319; Itgb3bp | 67733; Kif11 | 16551; Kiz | 228730; Kntc1
208628; Map7d3 | 320923; Mcm6 | 17219; Mcmbp | 210711; Mei4 | 75033; Mis12 | 67139;
Mis18bp1 | 217653; Mnat1 | 17420; Ncapd2 | 68298; Ncapg | 54392; Ncaph | 215387; Ndc80 |
67052; Nde1 | 67203; Nek2 | 18005; Nek7 | 59125; Ninl | 78177; Pold1 | 18971; Prc1 |
233406; Racgap1 | 26934; Rec8 | 56739; Repin1 | 58887; Rfc1 | 19687

TABLE 19-18
Pro-Apoptosis (also see Table 19A-30 for human orthologs)

Aatk | 11302; Aen | 68048; Aifm2 | 71361; Apaf1 | 11783; Bex3 | 12070;
Bik | 12124; Casp2 | 12366; Casp8 | 12370; Casp9 | 12371; Cradd |
12905; Ddn | 13199; Fasl | 14103; Glipr1 | 73690; Gsdme | 54722;
Lgals7 | 16858; Pmaip1 | 58801; Prune2 | 353211; Rassf6 | 73246;
Stk17b | 98267

TABLE 19-19
mRNA Splicing (also see Table 19A-24 for human orthologs)

Alkbh5 | 268420; Ctnnbl1 | 66642; Khdrbs3 | 13992; Mbnl3 | 171170;
Rbm10 | 236732; Rbm11 | 224344; Rbm20 | 73713; Rbm24 | 666794;
Rsrc1 | 66880; Rtca | 66368

TABLE 19-20
Transcription Factors (also see Table 19A-34 for human orthologs)

Adnp | 11538; Aes | 14797; Aire | 11634; Arid3a | 13496; Arid5b | 71371; Ascl2 | 17173;
Atad2 | 70472; Atmin | 234776; Atn1 | 13498; Atoh8 | 71093; Basp1 | 70350; Batf2 | 74481;
Batf3 | 318319; Bhlha15 | 17341; Bhlhe40 | 20893; Bzw1 | 66882; Carf | 241066; Casz1 |
69743; Cbfb | 12400; Ccdc85b | 240514; Cebpb | 12608; Cited1 | 12705; Cited4 | 56222; Cry1
| 12952; Deaf1 | 380959; E2f7 | 52679; Eaf2 | 106389; Egr2 | 13654; Egr3 | 13655; Eid1 |
58521; Eid2 | 386655; Elf1 | 13709; Elk1 | 13712; Emx1 | 13796; En2 | 13799; Eomes | 13813;
Ets1 | 23871; Etv4 | 18612; Etv5 | 104156; Etv6 | 14011; Eya2 | 14049; Fank1 | 66930; Foxn1 |
15218; Foxn3 | 71375; Fubp1 | 51886; Gata1 | 14460; Gfi1b | 14582; Glis3 | 226075; Gm340 |
212391; Grhl1 | 195733; Gtf2ird1 | 57080; Gzf1 | 74533; Hes1 | 15205; Hesx1 | 15209; Hey1 |
15213; Hic2 | 58180; Hif1a | 15251; Hinfp | 102423; Hivep3 | 16656; Hlx | 15284; Hmx2 |
15372; Hnf1a | 21405; Hnf1b | 21410; Hoxa10 | 15395; Hoxa4 | 15401; Hoxa7 | 15404; Hoxa9
| 15405; Hoxb5 | 15413; Hoxb7 | 15415; Hoxc11 | 109663; Hoxc4 | 15423; Hoxc9 | 15427;
Hoxd3 | 15434; Hsf4 | 26386; Id2 | 15902; Irf2bp2 | 270110; Irf6 | 54139; Irx1 | 16371; Irx4 |
50916; Irx5 | 54352; Jun | 16476; Klf12 | 16597; Klf4 | 16600; Klf6 | 23849; Lcor | 212391;
Ldb1 | 16825; Lmo1 | 109594; Maff | 17133; Maz | 17188; Mbd1 | 17190; Mef2c | 17260;
Meis3 | 17537; Mier2 | 70427; Mitf | 17342; Mxd3 | 17121; Myb | 17863; Mybl1 | 17864;
Mybl2 | 17865; Myc | 17869; Mzf1 | 109889; Nab1 | 17936; Nanos1 | 332397; Nfe2 | 18022;
Nfia | 18027; Nfil3 | 18030; Nfkb1 | 18033; Nfkb2 | 18034; Nfxl1 | 100978; Nfyc | 18046;
Nkx1-2 | 20231; Onecut3 | 246086; Ovol1 | 18426; Pax2 | 18504; Pax8 | 18510; Pbx1 | 18514;
Pbx3 | 18516; Pcbp3 | 59093; Pcgf2 | 22658; Pim2 | 18715; Pknox1 | 18771; Pou2f2 | 18987;
Pou4f1 | 18996; Ppargc1b | 170826; Rnf10 | 50849; Sall1 | 58198; Sim1 | 20464; Sim2 |
20465; Sox7 | 20680; Spib | 272382; Tfap2c | 21420; Tfcp2l1 | 81879; Trp63 | 22061; Twist1 |
22160; Wt1 | 22431; Zbtb32 | 58206; Zeb1 | 21417; Zfp367 | 238673

TABLE 19-21
Hif1a Signaling Pathway (also see
Table 19A-9 for human orthologs)

Cybb | 13058; Rwdd3 | 66568; Hif1a | 15251; Mir210 | 387206; Pdk1 |
228026; Pdk3 | 236900

TABLE 19-22
WNT Signaling (also see Table 19A-36 for human orthologs)

Adgra2 | 78560; Apcdd1 | 494504; Dkk2 | 56811; Frat2 | 212398; Fzd5 | 14367; Fzd7 | 14369;
Islr | 26968; Lzts2 | 226154; Ryk | 20187

TABLE 19-23
General Mitochondria (also see Table 19A-22 for human orthologs)

Acad10 | 71985; Acad8 | 66948; Acadsb | 66885; Acp6 | 66659; Acsm2 | 233799; Adhfe1 |
76187; Agk | 69923; Agmat | 75986; Agxt2 | 268782; Aldh5a1 | 214579; Amt | 434437;
Armcx1 | 78248; Armcx2 | 67416; Armcx3 | 71703; Bckdhb | 12040; Bco2 | 170752; Ccdc58 |
381045; Clpb | 20480; Clybl | 69634; Cmc1 | 67899; Coa5 | 76178; Coq2 | 71883; Coq4 |
227683; Coq7 | 12850; Cryzl2 | 226527; Cyb5b | 66427; Cyp11a1 | 13070; Cyp27a1 | 104086;
Cyp27b1 | 13115; Cyp2u1 | 71519; Ddah1 | 69219; Dhodh | 56749; Dhtkd1 | 209692; Dmgdh |
74129; Efhd1 | 98363; Ethe1 | 66071; Fahd1 | 68636; Fahd2a | 68126; Fam173a | 214917;
Fam72a | 108900; Fastk | 66587; Gad1 | 14415; Ggnbp1 | 70772; Gk | 14933; Gls2 | 216456;
Glyat | 107146; Gpat2 | 215456; Gstk1 | 76263; Hemk1 | 69536; Hint2 | 68917; Hoga1 |
67432; Iba57 | 216792; Immp2l | 93757; Ivd | 56357; Kmo | 98256; Ldhd | 52815; Lipt1 |
623661; Macrod1 | 107227; Maoa | 17161; Mccc2 | 78038; Mcee | 73724; Mcub | 11839; Me3
| 109264; Mettl15 | 76894; Micu3 | 78506; Mpst | 246221; Mrs2 | 380836; Mthfd2l | 665563;
Naxe | 246703; Nme4 | 56520; Nmnat3 | 74080; Pcca | 110821; Pccb | 66904; Pcx | 18563;
Pde12 | 211948; Pgs1 | 74451; Plekhn1 | 231002; Plscr3 | 70310; Ppox | 19044; Pptc7 |
320717; Prodh | 19125; Prodh2 | 56189; Pycr1 | 209027; Rexo2 | 104444; Sfxn2 | 94279

TABLE 19-24
Glycolysis (also see Table 19A-8 for human orthologs)

Aldoa | 11674; Aldob | 230163; Aldoc | 11676; Eno1 | 13806; Gpi1 | 14751; Hk2 | 15277;
Hkdc1 | 216019; Ldha | 16828; Pfkfb3 | 170768; Pfkp | 56421; Pgk1 | 18655; Pkm | 18746;
Slc2a1 | 20525; Tpi1 | 21991

TABLE 19-25
Oxidative Phosphorylation (also see Table 19A-26 for human orthologs)

Atp5a1 | 11946; Atp5b | 11947; Atp5d | 66043; Atp5f1 | 11950; Atp5g1 | 11951; Atp5g2 |
67942; Atp5g3 | 228033; Atp5h | 71679; Atp5j | 11957; Atp5j2 | 57423; Atp5o | 28080; Atp5s
| 68055; Bcs1l | 66821; Cep89 | 72140; Coa3 | 52469; Coa4 | 68185; Coa5 | 76178; Coa7 |
69893; Cox10 | 70383; Cox11 | 69802; Cox14 | 66379; Cox15 | 226139; Cox16 | 66272;
Cox17 | 12856; Cox18 | 231430; Cox19 | 68033; Cox20 | 66359; Cox4i1 | 12857; Cox4i2 |
84682; Cox5a | 12858; Cox6a1 | 12861; Cox6b1 | 110323; Cox7a1 | 12865; Cox7a2 | 12866;
Cox7a2l | 20463; Cox7b | 66142; Cox8a | 12868; Cyc1 | 66445; Cycs | 13063; Gm9763 |
100042918; LOC102631912 | 102631912; LOC102640946 | 102640946; Ndufa1 | 54405;
Ndufa10 | 67273; Ndufa11 | 69875; Ndufa12 | 66414; Ndufa13 | 67184; Ndufa2 | 17991;
Ndufa3 | 66091; Ndufa7 | 66416; Ndufa8 | 68375; Ndufa9 | 66108; Ndufab1 | 70316; Ndufaf1 |
69702; Ndufaf2 | 75597; Ndufaf3 | 66706; Ndufaf4 | 68493; Ndufaf5 | 69487; Ndufaf6 |
76947; Ndufaf7 | 73694; Ndufb10 | 68342; Ndufb11 | 104130; Ndufb2 | 68198; Ndufb3 |
66495; Ndufb4 | 68194; Ndufb5 | 66046; Ndufb6 | 230075; Ndufb7 | 66916; Ndufb8 | 67264;
Ndufb9 | 66218; Ndufc1 | 66377; Ndufc2 | 68197; Ndufs1 | 227197; Ndufs2 | 226646; Ndufs3
| 68349; Ndufs4 | 17993; Ndufs6 | 407785; Ndufs7 | 75406; Ndufs8 | 225887; Ndufv1 | 17995;
Ndufv2 | 72900; Ndufv3 | 78330; Nubpl | 76826; Oxa1l | 69089; Sco2 | 100126824; Slc25a4 |
11739; Timmdc1 | 76916; Tmem126b | 68472; Trap1 | 68015; Uqcc1 | 56046; Uqcc2 | 67267;
Uqcr10 | 66152; Uqcr11 | 66594; Uqcrb | 67530; Uqcrc1 | 22273; Uqcrc2 | 67003; Uqerfs1 |
66694; Uqcrh | 66576; Uqcrq | 22272

TABLE 19-26
Amino Acid Metabolism (also see Table 19A-1 for human orthologs)

Aass | 30956; Agxt | 11611; Agxt2 | 268782; Aldh4a1 | 212647; Aldh7a1 | 110695; Arg1 |
11846; Glud1 | 14661; Got1 | 14718; Gpt2 | 108682; Grhpr | 76238; Ivd | 56357; Mccc2 |
78038; Oat | 18242; Otc | 18416; Oxct1 | 67041; Pcx | 18563; Psat1 | 107272; Sds | 231691

TABLE 19-27
Fatty Acid Oxidation (also see Table 19A-6 for human orthologs)

Abcd1 | 11666; Abcd2 | 26874; Acaa2 | 52538; Acacb | 100705; Acad11 | 102632; Acadl |
11363; Acadm | 11364; Acadvl | 11370; Acat1 | 110446; Acat3 | 224530; Acox1 | 11430;
Acox2 | 93732; Acoxl | 74121; Acsbg2 | 328845; Adipoq | 11450; Akt2 | 11652; Auh | 11992;
Bdh2 | 69772; Cpt1a | 12894; Cpt2 | 12896; Decr1 | 67460; Echdc2 | 52430; Echs1 | 93747;
Eci2 | 23986; Ehhadh | 74147; Etfb | 110826; Etfdh | 66841; Fabp1 | 14080; Gcdh | 270076;
Haao | 107766; Hacl1 | 56794; Hadh | 15107; Hadha | 97212; Hadhb | 231086; Hibch |
227095; Hsd17b4 | 15488; Irs1 | 16367; Ivd | 56357; Lep | 16846; Mlycd | 56690; Pex13 |
72129; Pex2 | 19302; Pex5 | 19305; Pex7 | 18634; Plin5 | 66968; Ppard | 19015; Pparg | 19016;
Ppargc1a | 19017; Sesn2 | 230784; Slc27a2 | 26458; Twist1 | 22160

TABLE 19-28
Lipid Metabolism (also see Table 19A-17 for human orthologs)

Aadac | 67758; Abhd6 | 6082; Acly | 104112; Acsl5 | 433256; Angptl3 | 30924; Angptl4 |
57875; Apoa4 | 11808; Apoa5 | 66113; Apoe | 11816; Apoh | 11818; Ccdc3 | 74186; Cnep1r1 |
382030; Dbi | 13167; Dgat1 | 13350; Dgat2 | 67800; Disp3 | 242748; Fabp3 | 14077; Gpam |
14732; Gpld1 | 14756; Hsd17b13 | 243168; Ldlr | 16835; Mid1ip1 | 68041; Nr1h2 | 22260;
Nr1h3 | 22259; Pcx | 18563; Pnpla2 | 66853; Ppara | 19013; Slc27a1 | 26457; Srebf1 | 20787

TABLE 19-29
Podocyte (also see Table 19A-29 for human orthologs)

Nphs1 | 54631; Nphs2 | 170484; Podxl | 27205; Synpo | 104027; Wt1 | 22431

TABLE 19-30
Kidney Cell (also see Table 19A-12 for human orthologs)

Acot4 | 171282; Acsm2 | 233799; Agxt2 | 268782; Aldh4a1 | 212647; Bbox1 | 170442;
Ccdc198 | 67082; Cd24a | 12484; Cdh16 | 12556; Chst9 | 71367; Cldn10 | 58187; Cldn16 |
114141; Cldn2 | 12738; Cmbl | 69574; Col4a3 | 12828; Col4a4 | 12829; Col4a5 | 12830; Ctxn3
| 629147; Cxcl14 | 57266; Cyp27b1 | 13115; Cyp4a12a | 13118; Dao | 13142; Dcdc2a |
195208; Dmgdh | 74129; Dnm3 | 103967; Epcam | 17075; Esm1 | 71690; Etnk2 | 214253;
Fam151a | 230579; G6pc | 14377; Galnt14 | 71685; Glyat | 107146; Gne | 50798; Hey1 |
15213; Hnf1b | 21410; Hoga1 | 67432; Hoxd3 | 15434; Hpd | 15445; Hs3st1 | 15476; Hspa12a
| 73442; Hykk | 235386; Itgb8 | 320910; Kif12 | 16552; Lamb2 | 16779; Nat8 | 68396; Nr1h4 |
20186; P3h2 | 210530; Pank1 | 75725; Pax8 | 18510; Pck1 | 18534; Pipox | 19193; Pkhd1 |
241035; Pllp | 67801; Prodh | 19125; Prodh2 | 56189; Prom1 | 19126; Proz | 66901; Rab11fip3
| 215445; Rasip1 | 69903; Ren1 | 19701; Rhbg | 58176; Rhcg | 56315; Rnf152 | 320311;
Rnf183 | 76072; Sall1 | 58198; Sfxn2 | 94279; Sgk2 | 27219; Shisa3 | 330096; Sim2 | 20465;
Slc13a3 | 114644; Slc16a4 | 229699; Slc17a1 | 20504; Slc17a3 | 105355; Slc22a6 | 18399;
Slc22a7 | 108114; Slc30a2 | 230810; Slc34a3 | 142681; Slc3a1 | 20532; Slc7a13 | 74087;
Slc7a7 | 20540; Sod3 | 20657; Sostdc1 | 66042; Sult1c2 | 69083; Tmem174 | 68344; Tmem207
| 100043057; Tmprss4 | 214523; Trabd2b | 666048; Ugt1a2 | 22236; Ugt1a7c | 394432;
Ugt2b37 | 1112417; Ugt2b38 | 100559; Ugt2b5 | 22238; Ugt3a1 | 105887; Ugt3a2 | 223337;
Ugt8a | 22239; Upk1b | 22268; Wnk4 | 69847

TABLE 19-31
Kidney Distal Tubule (also see Table 19A-13 for human orthologs)

Atp6v0d2 | 242341; Atp6v1g3 | 338375; Calb1 | 12307; Cdh1 | 12550; Fxyd2 | 11936; Kcnj1 |
56379; Kcnj16 | 16517; Ppp1r1a | 58200; Pth1r | 19228; Pvalb | 19293; Slc12a1 | 20495;
Slc12a3 | 20497; Tmem72 | 319776; Umod | 22242; Wfdc2 | 67701

TABLE 19-32
Kidney Proximal Tubule (also see Table 19A-15 for human orthologs)

Acy1 | 109652; Aqp1 | 11826; Ass1 | 11898; Cltrn | 57394; Cryaa | 12954; Cryab | 12955;
Cubn | 65969; Ddah1 | 69219; Fmo1 | 14261; Gpx3 | 14778; Hnf1a | 21405; Miox | 56727;
Nox4 | 50490; Pah | 18478; Pax2 | 18504; Pdzk1 | 59020; Pdzk1ip1 | 67182; Slc13a1 | 55961;
Slc22a12 | 20521; Slc22a13 | 102570; Slc22a2 | 20518; Slc22a8 | 19879; Slc34a1 | 20505;
Slc36a2 | 246049; Slc4a4 | 54403; Slc5a2 | 246787; Slc6a18 | 22598

TABLE 19-33
Kidney Loop of Henle (also see Table
19A-14 for human orthologs)

Clcnka | 12733; Clcnkb | 56365; Kng1 | 16644

TABLE 19-34
Kidney Tubule Collecting Duct (also see Table 19A-16 for human orthologs)

Aqp2 | 1127; Cldn4 | 12740; Cldn8 | 54420; Efhd1 | 98363; Fxyd4 | 108017; Glis3 | 226075;
Mitf | 17342; Pigr | 18703; Scin | 20259; Scnn1a | 20276; Sim1 | 20464; Slc4a9 | 240215;
Tfcp2l1 | 81879; Wwc1 | 211652

TABLE 19-35
Androgen Regulated Genes (also see Table 19A-2 for human orthologs)

Akap1 | 11640; Ccdc136 | 232664; Cox6b1 | 110323; Cst3 | 13010; Iapp | 15874; Klk1 |
16612; Klkb1 | 16621; Mrps6 | 121022; Mybbp1a | 18432; Ndufa1 | 54405; Nkx3-1 | 18095;
Nup37 | 69736; Phkg2 | 68961; Prelid1 | 66494; Sord | 20322; St3gal5 | 20454; Tmem86a |
67893

TABLE 19-36
Estrogen Regulated Genes (also see Table 19A-5 for human orthologs)

Abcb6 | 74104; Adam12 | 11489; Aebp1 | 11568; Aen | 68048; Aimp2 | 231872; Ammecr1 |
56068; Amn | 93835; Ank1 | 11733; Arhgap24 | 231532; Asic2 | 11418; Asns | 27053; Bcan |
12032; Cacna1a | 12286; Cckar | 12425; Cep89 | 72140; Cgref1 | 68567; Cited1 | 12705; Dbh |
13166; Fbxo31 | 76454; Fbxw7 | 50754; G6pd2 | 14380; G6pdx | 14381; Gadd45g | 23882;
Ggt5 | 23887; Hif1a | 15251; Hspb8 | 80888; Immt | 76614; Impdh1 | 23917; Impg1 | 63859;
Kcne2 | 246133; Lamb3 | 16780; Lancl3 | 236285; Mcm2 | 17216; Mesd | 67943; Mfge8 |
17304; Mogs | 57377; Myc | 17869; Naaa | 67111; Nhlh2 | 18072; Nomo1 | 211548; Nop56 |
67134; Ntm | 235106; Nudt19 | 110959; Opn3 | 13603; Oxtr | 18430; Pcsk6 | 18553; Perp |
64058; Pgm2 | 72157; Phyhipl | 70911; Plod1 | 18822; Prmt3 | 71974; Ptpn5 | 19259; Rnd2 |
11858; Scnn1g | 20278; Slc10a3 | 214601; Srebf1 | 20787; Stat5a | 20850; Tbl3 | 213773;
Tcam1 | 75870; Tmem86a | 67893; Twnk | 226153; Zfp804a | 241514

Tables 19A-1-19A-36: Human orthologs of the mouse genes listed in Table 19-1 to 19-36. The genes are listed by Gene symbol I Entrez ID.
The genes in each of Tables 19A-1 to 19A-36 can be used as effective biomarkers for classifying the LN disease state of the patient.

TABLE 19A-1
Amino Acid Metabolism

AASS | 10157; AGXT | 189; AGXT2 | 64902; ALDH4A1 | 8659; ALDH7A1 | 501; ARG1 | 383;
GLUD1 | 2746; GOT1 | 2805; GPT2 | 84706; GRHPR | 9380; IVD | 3712; MCCC2 | 64087; OAT
| 4942; OTC | 5009; OXCT1 | 5019; PC | 5091; PSAT1 | 29968; SDS | 10993;

TABLE 19A-2
Androgen_Regulated_Genes

AKAP1 | 8165; CCDC136 | 64753; COX6B1 | 1340; CST3 | 1471; IAPP | 3375; KLK1 | 3816;
KLKB1 | 3818; MRPS6 | 64968; MYBBP1A | 10514; NDUFA1 | 4694; NKX3-1 | 4824; NUP37 |
79023; PHKG2 | 5261; PRELID1 | 27166; SORD | 6652; ST3GAL5 | 8869; TMEM86A | 144110;

TABLE 19A-3
Antigen Presenting Cell

CD1D | 912; CD74 | 972; CIITA | 4261; CLEC10A | 10462; HLA-DMA | 3108; HLA-DMB |
3109; HLA-DOA | 3111; HLA-DOB | 3112; HLA-DQA1 | 3117; HLA-DQB1 | 3119; HLA-DRA |
3122; HLA-DRB5 | 3127; RFX5 | 5993; SCIMP | 388325;

TABLE 19A-4
CD8 T cell

CD8A | 925; CD8B | 926;

TABLE 19A-5
Estrogen_Regulated_Genes

ABCB6 | 10058; ADAM12 | 8038; AEBP1 | 165; AEN | 64782; AIMP2 | 7965; AMMECR1 |
9949; AMN | 81693; ANK1 | 286; ARHGAP24 | 83478; ASIC2 | 40; ASNS | 440; BCAN | 63827;
CACNA1A | 773; CCKAR | 886; CEP89 | 84902; CGREF1 | 10669; CITED1 | 4435; DBH | 1621;
FBXO31 | 79791; FBXW7 | 55294; G6PD | 2539; GADD45G | 10912; GGT5 | 2687; HIF1A |
3091; HSPB8 | 26353; IMMT | 10989; IMPDH1 | 3614; IMPG1 | 3617; KCNE2 | 9992; LAMB3 |
3914; LANCL3 | 347404; MCM2 | 4171; MESD | 23184; MFGE8 | 4240; MOGS | 7841; MYC |
4609; NAAA | 27163; NHLH2 | 4808; NOMO2 | 283820; NOP56 | 10528; NTM | 50863;
NUDT19 | 390916; OPN3 | 23596; OXTR | 5021; PCSK6 | 5046; PERP | 64065; PGM2 | 55276;
PHYHIPL | 84457; PLOD1 | 5351; PRMT3 | 10196; PTPN5 | 84867; RND2 | 8153; SCNN1G |
6340; SLC10A3 | 8273; SREBF1 | 6720; STAT5A | 6776; TBL3 | 10607; TMEM86A | 144110;
TWNK | 56652; ZNF804A | 91752;

TABLE 19A-1
Amino Acid Metabolism

AASS | 10157; AGXT | 189; AGXT2 | 64902; ALDH4A1 | 8659;
ALDH7A1 | 501; ARG1 | 383; GLUD1 | 2746; GOT1 | 2805; GPT2 |
84706; GRHPR | 9380; IVD | 3712; MCCC2 | 64087; OAT | 4942;
OTC | 5009; OXCT1 | 5019; PC | 5091; PSAT1 | 29968; SDS | 10993;

TABLE 19A-7
GC B Cell

FCRLB | 127943; GCSAM | 257144; KLHL6 | 89857; MYBL1 | 4603;
NUGGC | 389643; RGS13 | 6003;

TABLE 19A-8
Glycolysis

ALDOA | 226; ALDOB | 229; ALDOC | 230; ENO1 | 2023; GPI | 2821;
HK2 | 3099; HKDC1 | 80201; LDHA | 3939; PFKFB3 | 5209; PFKP |
5214; PGK1 | 5230; PKM | 5315; SLC2A1 | 6513; TPI1 | 7167;

TABLE 19A-9
Hif1a Signaling

CYBB | 1536; HIF1A | 3091; PDK1 | 5163; PDK3 | 5165; RWDD3 |
25950;

TABLE 19A-10
IFN Stimulated Genes

EIF2AK2 | 5610; GBP2 | 2634; GBP6 | 163351; IFI16 | 3428;
IFI30 | 10437; IFI44 | 10561; IFITM1 | 8519; IRGM | 345611;
MNDA | 4332; MX1 | 4599; PARP9 | 83666; RSAD2 | 91543;

TABLE 19A-11
IG Chain Cell

IGHA1 | 3493; IGHA2 | 3494; IGHE | 3497; IGHG1 | 3500; IGHG2 | 3501; IGHG3 | 3502;
IGHG4 | 3503; IGHM | 3507; IGHV1-18 | 28468; IGHV1-24 | 28467; IGHV1-3 | 28473; IGHV1-
45 | 28466; IGHV1-46 | 28465; IGHV1-58 | 28464; IGHV1-69 | 28461; IGHV1-69-2 | 28458;
IGHV1-69D | 102723169; IGHV1OR15-9 | 390531; IGHV1OR21-1 | 390530; IGHV2-26 | 28455;
IGHV2-5 | 28457; IGHV2-70 | 28454; IGHV2-70D | 102723168; IGHV2OR16-5 | 100129631;
IGHV3-72 | 28410; IGHV3-73 | 28409; IGHV4-28 | 28400; IGHV4-31 | 28396; IGHV4-34 |
28395; IGHV4-39 | 28394; IGHV4-4 | 28401; IGHV4-59 | 28392; IGHV4-61 | 28391;
IGHV4OR15-8 | 28317; IGHV7-4-1 | 57289; IGHV7-81 | 28378; IGKC | 3514; IGKV1-16 |
28938; IGKV1-17 | 28937; IGKV1-33 | 28933; IGKV1-37 | 28931; IGKV1-39 | 28930; IGKV1-5
| 28299; IGKV1D-17 | 28900; IGKV1D-33 | 28896; IGKV1D-37 | 28894; IGKV1D-39 | 28893;
IGKV1D-43 | 28891; IGKV2-30 | 28919; IGKV2D-29 | 28882; IGKV2D-30 | 28881; IGKV3-7 |
28915; IGKV3D-11 | 28876; IGKV3D-15 | 28875; IGKV3D-20 | 28874; IGKV3D-7 | 28877;
IGKV4-1 | 28908; IGKV6-21 | 28906; IGKV6D-21 | 28870; IGLC1 | 3537; IGLC2 | 3538; IGLC3
| 3539; IGLC7 | 28834; IGLV4-3 | 28786; IGLV4-60 | 28785; IGLV4-69 | 28784;

TABLE 19A-12
Kidney Cell

ACOT4 | 122970; ACSM2A | 123876; AGXT2 | 64902; ALDH4A1 | 8659; BBOX1 | 8424; CD24
| 100133941; CDH16 | 1014; CHST9 | 83539; CLDN10 | 9071; CLDN16 | 10686; CLDN2 | 9075;
CMBL | 134147; COL4A3 | 1285; COL4A4 | 1286; COL4A5 | 1287; CTXN3 | 613212; CXCL14 |
9547; CYP27B1 | 1594; CYP4A22 | 284541; DAO | 1610; DCDC2 | 51473; DMGDH | 29958;
DNM3 | 26052; EPCAM | 4072; ESM1 | 11082; ETNK2 | 55224; FAM151A | 338094; G6PC1 |
2538; GALNT14 | 79623; GLYAT | 10249; GNE | 10020; HEY1 | 23462; HNF1B | 6928;
HOGA1 | 112817; HOXD3 | 3232; HPD | 3242; HS3ST1 | 9957; HSPA12A | 259217; HYKK |
123688; ITGB8 | 3696; KIF12 | 113220; LAMB2 | 3913; NAT8 | 9027; NR1H4 | 9971; P3H2 |
55214; PANK1 | 53354; PAX8 | 7849; PCK1 | 5105; PIPOX | 51268; PKHD1 | 5314; PLLP |
51090; PRODH | 5625; PRODH2 | 58510; PROM1 | 8842; PROZ | 8858; RAB11FIP3 | 9727;
RASIP1 | 54922; REN | 5972; RHBG | 57127; RHCG | 51458; RNF152 | 220441; RNF183 |
138065; SALL1 | 6299; SFXN2 | 118980; SGK2 | 10110; SHISA3 | 152573; SIM2 | 6493;
SLC13A3 | 64849; SLC16A4 | 9122; SLC17A1 | 6568; SLC17A3 | 10786; SLC22A6 | 9356;
SLC22A7 | 10864; SLC30A2 | 7780; SLC34A3 | 142680; SLC3A1 | 6519; SLC7A13 | 157724;
SLC7A7 | 9056; SOD3 | 6649; SOSTDC1 | 25928; SULT1C2 | 6819; TMEM174 | 134288;
TMEM207 | 131920; TMPRSS4 | 56649; TRABD2B | 388630; UGT1A4 | 54657; UGT1A9 |
54600; UGT2B28 | 54490; UGT3A1 | 133688; UGT3A2 | 167127; UGT8 | 7368; UPK1B | 7348;
WNK4 | 65266;

TABLE 19A-13
Kidney Distal Tubule

ATP6V0D2 | 245972; ATP6V1G3 | 127124; CALB1 | 793; CDH1 | 999;
FXYD2 | 486; KCNJ1 | 3758; KCNJ16 | 3773; PPP1R1A | 5502; PTH1R |
5745; PVALB | 5816; SLC12A1 | 6557; SLC12A3 | 6559; TMEM72 |
643236; UMOD | 7369; WFDC2 | 10406;

TABLE 19A-14
Kidney Loop of Henle

CLCNKA | 1187; CLCNKB | 1188; CLCNKB | 1188; KNG1 | 3827;

TABLE 19A-15
Kidney Prox Convoluted Tubule

ACY1 | 95; AQP1 | 358; ASS1 | 445; CLTRN | 57393; CRYAA | 1409; CRYAB | 1410; CUBN |
8029; DDAH1 | 23576; FMO1 | 2326; GPX3 | 2878; HNF1A | 6927; MIOX | 55586; NOX4 |
50507; PAH | 5053; PAX2 | 5076; PDZK1 | 5174; PDZK1IP1 | 10158; SLC13A1 | 6561;
SLC22A12 | 116085; SLC22A13 | 9390; SLC22A2 | 6582; SLC22A8 | 9376; SLC34A1 | 6569;
SLC36A2 | 153201; SLC4A4 | 8671; SLC5A2 | 6524; SLC6A18 | 348932;

TABLE 19A-16
Kidney Tubule Collecting Duct

AQP2 | 359; CLDN4 | 1364; CLDN8 | 9073; EFHD1 | 80303; FXYD4 |
53828; GLIS3 | 169792; MITF | 4286; PIGR | 5284; SCIN | 85477;
SCNN1A | 6337; SIM1 | 6492; SLC4A9 | 83697; TFCP2L1 | 29842;
WWC1 | 23286;

TABLE 19A-17
Lipid Metabolism

AADAC | 13; ABHD6 | 57406; ACLY | 47; ACSL5 | 51703; ANGPTL3 | 27329; ANGPTL4 |
51129; APOA4 | 337; APOA5 | 116519; APOE | 348; APOH | 350; CCDC3 | 83643; CNEP1R1 |
255919; DBI | 1622; DGAT1 | 8694; DGAT2 | 84649; DISP3 | 57540; FABP3 | 2170; GPAM |
57678; GPLD1 | 2822; HSD17B13 | 345275; LDLR | 3949; MID1IP1 | 58526; NR1H2 | 7376;
NR1H3 | 10062; PC | 5091; PNPLA2 | 57104; PPARA | 5465; SLC27A1 | 376497; SREBF1 |
6720;

TABLE 19A-18
M1 Macrophage

AOAH | 313; ARHGAP24 | 83478; CCRL2 | 9034; CD300LF | 146722; CD38 | 952; CFB | 629;
CP | 1356; CPD | 1362; CXCL10 | 3627; DDX58 | 23586; DDX60 | 55601; EBI3 | 10148; F11R |
50848; FPR1 | 2357; FPR2 | 2358; GBP6 | 163351; GNGT2 | 2793; GPR18 | 2841; HERC6 |
55008; HLA-A | 3105; HLA-E | 3133; HLA-F | 3134; HP | 3240; IFI44 | 10561; IFIT2 | 3433;
IRAK3 | 11213; ISG15 | 9636; ITGAL | 3683; MARCO | 8685; MS4A4A | 51338; MX1 | 4599;
NFKBIZ | 64332; OASL | 8638; PSTPIP2 | 9050; RSAD2 | 91543; SLFN12 | 55106; SLFN12L |
100506736; SLFN13 | 146857; STAT1 | 6772; STAT2 | 6773; TLR2 | 7097; TUBA4A | 7277;
XAF1 | 54739; ZBP1 | 81030;

TABLE 19A-19
M2 Macrophage

ACTN1 | 87; AMZ1 | 155185; ATP6V0A1 | 535; ATP6V0D2 | 245972; BCAR3 | 8412;
CD300LD | 100131439; CD83 | 9308; CHST7 | 56548; CLEC10A | 10462; CLEC7A | 64581;
EGR2 | 1959; EMP2 | 2013; FLRT2 | 23768; GNB4 | 59345; IL6ST | 3572; LMNA | 4000; MATK
| 4145; MMP12 | 4321; MMP9 | 4318; MRC1 | 4360; MYC | 4609; OLFM1 | 10439; P2RY1 |
5028; PLK2 | 10769; PTGS1 | 5742; RHOJ | 57381; SOCS6 | 9306; TANC2 | 26115; TIAM1 |
7074; TMEM158 | 25907; VWF | 7450;

TABLE 19A-20
MHC-Class-ONE

B2M | 567; CD1D | 912; ERAP1 | 51752; FCGRT | 2217; HLA-A |
3105; HLA-E | 3133; HLA-F | 3134; HLA-G | 3135; MICB | 4277; MR1
| 3140; TAP1 | 6890; TAP2 | 6891; TAPBP | 6892; TAPBPL | 55080;

TABLE 19A-21
MHC-Class-TWO

CD74 | 972; CIITA | 4261; HLA-DMA | 3108; HLA-DMB | 3109;
HLA-DOA | 3111; HLA-DQA1 | 3117; HLA-DQB1 | 3119; HLA-DRB5 |
3127; RFX5 | 5993;

TABLE 19A-22
Mitochondria General

ACAD10 | 80724; ACAD8 | 27034; ACADSB | 36; ACP6 | 51205; ACSM2A | 123876; ADHFE1
| 137872; AGK | 55750; AGMAT | 79814; AGXT2 | 64902; ALDH5A1 | 7915; AMT | 275;
ARMCX1 | 51309; ARMCX2 | 9823; ARMCX3 | 51566; BCKDHB | 594; BCO2 | 83875; CLPB |
81570; CLYBL | 171425; CMC1 | 152100; COA5 | 493753; COQ2 | 27235; COQ4 | 51117;
COQ7 | 10229; CYB5B | 80777; CYP11A1 | 1583; CYP27A1 | 1593; CYP27B1 | 1594; CYP2U1
| 113612; DDAH1 | 23576; DHODH | 1723; DHTKD1 | 55526; DMGDH | 29958; EFHD1 |
80303; ETHE1 | 23474; FAHD1 | 81889; FAHD2A | 51011; FAHD2B | 151313; FAM72A |
729533; FASTK | 10922; GAD1 | 2571; GK | 2710; GLS2 | 27165; GLYAT | 10249; GPAT2 |
150763; GSTK1 | 373156; HEMK1 | 51409; HINT2 | 84681; HOGA1 | 112817; IBA57 | 200205;
IMMP2L | 83943; IVD | 3712; KMO | 8564; LDHD | 197257; LIPT1 | 51601; MACROD1 |
28992; MAOA | 4128; MCCC2 | 64087; MCEE | 84693; MCUB | 55013; ME3 | 10873;
METTL15 | 196074; MICU3 | 286097; MIX23 | 131076; MPST | 4357; MRS2 | 57380;
MTHFD2L | 441024; NAXE | 128240; NME4 | 4833; NMNAT3 | 349565; PC | 5091; PCCA |
5095; PCCB | 5096; PDE12 | 201626; PGS1 | 9489; PLEKHN1 | 84069; PLSCR3 | 57048; PPOX |
5498; PPTC7 | 160760; PRODH | 5625; PRODH2 | 58510; PYCR1 | 5831; REXO2 | 25996;
SFXN2 | 118980;

TABLE 19A-23
Monocyte-Macrophage

ACE | 1636; ADAM8 | 101; ADGRE1 | 2015; APOBR | 55911; APOC1 | 341; C1QA | 712;
C1QB | 713; C1QC | 714; C2 | 717; C4B | 721; C6 | 729; CCL7 | 6354; CCL8 | 6355; CD14 | 929;
CD163 | 9332; CD300E | 342510; CD5L | 922; CFB | 629; CFD | 1675; CFP | 5199; CSF1R |
1436; CXCL13 | 10563; FOLR2 | 2350; IFI16 | 3428; IL31RA | 133396; LGALS12 | 85329;
LYVE1 | 10894; MARCO | 8685; MEGF10 | 84466; MERTK | 10461; MFGE8 | 4240; MRC1 |
4360; MS4A4A | 51338; MSR1 | 4481; PLA2G5 | 5322; SCARF2 | 91179; SIGLEC1 | 6614;

TABLE 19A-24
mRNA Splicing

ALKBH5 | 54890; CTNNBL1 | 56259; KHDRBS3 | 10656; MBNL3 |
55796; RBM10 | 8241; RBM11 | 54033; RBM20 | 282996; RBM24 |
221662; RSRC1 | 51319; RTCA | 8634;

TABLE 19A-25
Myeloid Cell

AIF1 | 199; AOAH | 313; ARG1 | 383; BATF2 | 116071; BMX | 660; BPI | 671; CD300LF |
146722; CD33 | 945; CD68 | 968; CLEC12A | 160364; CLEC4A | 50856; CLEC4C | 170482;
CLEC4D | 338339; CLEC4E | 26253; CLEC4G | 339390; CLEC5A | 23601; CLEC6A | 93978;
CLEC7A | 64581; CSF2RA | 1438; CSF2RB | 1439; CSF3R | 1441; CST3 | 1471; CXCL9 | 4283;
CYBA | 1535; CYBB | 1536; FCER1A | 2205; FCGR1A | 2209; FCGR2A | 2212; FCGR3A |
2214; FFAR2 | 2867; FGR | 2268; IGSF6 | 10261; IL1RN | 3557; LILRB4 | 11006; LYZ | 4069;
MPEG1 | 219972; MS4A6A | 64231; NLRP12 | 91662; NLRP3 | 114548; OLR1 | 4973; P2RX5 |
5026; PILRA | 29992; PLA2G2D | 26279; S100A9 | 6280; SCARB1 | 949; SERPING1 | 710;
SLAMF8 | 56833; TREM1 | 54210; TREML4 | 285852; VSIG4 | 11326;

TABLE 19A-26
Oxidative Phosphorylation

ATP5F1A | 498; ATP5F1B | 506; ATP5F1D | 513; ATP5MC1 | 516; ATP5MC2 | 517; ATP5MC3 |
518; ATP5MF | 9551; ATP5PD | 10476; ATP5PF | 522; ATP5PO | 539; BCS1L | 617; CEP89 |
84902; COA3 | 28958; COA4 | 51287; COA5 | 493753; COA7 | 65260; COX10 | 1352; COX11 |
1353; COX14 | 84987; COX15 | 1355; COX16 | 51241; COX17 | 10063; COX18 | 285521;
COX19 | 90639; COX20 | 116228; COX4I1 | 1327; COX4I2 | 84701; COX5A | 9377; COX6A1 |
1337; COX6B1 | 1340; COX7A1 | 1346; COX7A2 | 1347; COX7A2L | 9167; COX7B | 1349;
COX8A | 1351; CYC1 | 1537; CYCS | 54205; NDUFA1 | 4694; NDUFA10 | 4705; NDUFA11 |
126328; NDUFA12 | 55967; NDUFA13 | 51079; NDUFA2 | 4695; NDUFA3 | 4696; NDUFA7 |
4701; NDUFA8 | 4702; NDUFA9 | 4704; NDUFAB1 | 4706; NDUFAF1 | 51103; NDUFAF2 |
91942; NDUFAF3 | 25915; NDUFAF4 | 29078; NDUFAF5 | 79133; NDUFAF6 | 137682;
NDUFAF7 | 55471; NDUFB10 | 4716; NDUFB11 | 54539; NDUFB2 | 4708; NDUFB3 | 4709;
NDUFB4 | 4710; NDUFB5 | 4711; NDUFB6 | 4712; NDUFB7 | 4713; NDUFB8 | 4714; NDUFB9 |
4715; NDUFC1 | 4717; NDUFC2 | 4718; NDUFS1 | 4719; NDUFS2 | 4720; NDUFS3 | 4722;
NDUFS4 | 4724; NDUFS6 | 4726; NDUFS7 | 374291; NDUFS8 | 4728; NDUFV1 | 4723;
NDUFV2 | 4729; NDUFV3 | 4731; NUBPL | 80224; OXA1L | 5018; SCO2 | 9997; SLC25A4 |
291; TIMMDC1 | 51300; TMEM126B | 55863; TRAP1 | 10131; UQCC1 | 55245; UQCC2 |
84300; UQCR10 | 29796; UQCR11 | 10975; UQCRB | 7381; UQCRC1 | 7384; UQCRC2 | 7385;
UQCRFS1 | 7386; UQCRH | 7388; UQCRQ | 27089;

TABLE 19A-27
Pattern-Recognition-Receptors

AGER | 177; ALPK1 | 80216; APOBEC3H | 164668; CD209 | 30835; CLEC4D | 338339;
CLEC4E | 26253; CLEC7A | 64581; DDX58 | 23586; IFI16 | 3428; IRAK4 | 51135; IRF7 | 3665;
IRF8 | 3394; IRF9 | 10379; LRRFIP1 | 9208; MARCO | 8685; MEFV | 4210; NLRC3 | 197358;
NLRC5 | 84166; NLRP10 | 338322; NLRP12 | 91662; NLRP3 | 114548; NOD2 | 64127; OAS2 |
4939; OAS3 | 4940; PGLYRP1 | 8993; TLR1 | 7096; TLR5 | 7100; TLR7 | 51284; TLR9 | 54106;
UNC93B1 | 81622;

TABLE 19A-28
Platelet

CD9 | 928; CXCL6 | 6372; ESAM | 90952; GP1BA | 2811; GP5 | 2814;
GP6 | 51206; GP9 | 2815; LY6G6D | 58530; MMRN1 | 22915; MYL9 |
10398; PEAR1 | 375033; PF4 | 5196; PLEK | 5341; PPBP | 5473;
SLC35D3 | 340146; TREML1 | 340205; TSPAN9 | 10867;

TABLE 19A-29
Podocyte

NPHS1 | 4868; NPHS2 | 7827; PODXL | 5420; SYNPO | 11346; WT1 |
7490;

TABLE 19A-30
Pro-Apoptosis

AEN | 64782; AIFM2 | 84883; APAF1 | 317; BEX3 | 27018; BIK | 638; CASP2 | 835; CASP8 |
841; CASP9 | 842; CRADD | 8738; DDN | 23109; FASLG | 356; GLIPR1 | 11010; GSDME |
1687; LGALS7 | 3963; PMAIP1 | 5366; PRUNE2 | 158471; RASSF6 | 166824; STK17B | 9262;

TABLE 19A-31
Pro-Cell-Cycle

AURKB | 9212; BUB1 | 699; BUB1B | 701; CCDC88A | 55704; CCND2 | 894; CCNG1 | 900;
CDC20 | 991; CDC25B | 994; CDCA2 | 157313; CDCA5 | 113130; CDK14 | 5218; CDK5 | 1020;
CDK6 | 1021; CENPC | 1060; CEP55 | 55165; CLSPN | 63967; DBF4 | 10926; DSN1 | 79980;
E2F2 | 1870; E2F3 | 1871; ECT2 | 1894; ESCO2 | 157570; ESPL1 | 9700; FOXM1 | 2305;
INCENP | 3619; ITGB3BP | 23421; KIF11 | 3832; KIZ | 55857; KNTC1 | 9735; MAP7D3 |
79649; MCM6 | 4175; MCMBP | 79892; MEI4 | 101928601; MIS12 | 79003; MIS18BP1 | 55320;
MNAT1 | 4331; NCAPD2 | 9918; NCAPG | 64151; NCAPH | 23397; NDC80 | 10403; NDE1 |
54820; NEK2 | 4751; NEK7 | 140609; NINL | 22981; POLD1 | 5424; PRC1 | 9055; RACGAP1 |
29127; REC8 | 9985; REPIN1 | 29803; RFC1 | 5981;

TABLE 19A-32
Tfh Cell

ASCL2 | 430; BCL6 | 604; CXCR5 | 643; ICOS | 29851; NT5E |
4907; PDCD1 | 5133; TNFRSF4 | 7293;

TABLE 19A-33
Th17 Cell

AHR | 196; CD3D | 915; CD3E | 916; CD3G | 917; IL17A |
3605; IL17F | 112744; IL21 | 59067; IL22 | 50616; IRF4 | 3662;
NFXL1 | 152518; PGF | 5228; RORA | 6095; RORC | 6097; TRAF6 |
7189;

TABLE 19A-34
Transcription Factors

ADNP | 23394; AIRE | 326; ARID3A | 1820; ARID5B | 84159; ASCL2 | 430; ATAD2 | 29028;
ATMIN | 23300; ATN1 | 1822; ATOH8 | 84913; BASP1 | 10409; BATF2 | 116071; BATF3 |
55509; BHLHA15 | 168620; BHLHE40 | 8553; BZW1 | 9689; CARF | 79800; CASZ1 | 54897;
CBFB | 865; CCDC85B | 11007; CEBPB | 1051; CITED1 | 4435; CITED4 | 163732; CRY1 |
1407; DEAF1 | 10522; E2F7 | 144455; EAF2 | 55840; EGR2 | 1959; EGR3 | 1960; EID1 | 23741;
EID2 | 163126; ELF1 | 1997; ELK1 | 2002; EMX1 | 2016; EN2 | 2020; EOMES | 8320; ETS1 |
2113; ETV4 | 2118; ETV5 | 2119; ETV6 | 2120; EYA2 | 2139; FANK1 | 92565; FOXN1 | 8456;
FOXN3 | 1112; FUBP1 | 8880; GATA1 | 2623; GFI1B | 8328; GLIS3 | 169792; GRHL1 | 29841;
GTF2IRD1 | 9569; GZF1 | 64412; HES1 | 3280; HESX1 | 8820; HEY1 | 23462; HIC2 | 23119;
HIF1A | 3091; HINFP | 25988; HIVEP3 | 59269; HLX | 3142; HMX2 | 3167; HNF1A | 6927;
HNF1B | 6928; HOXA10 | 3206; HOXA4 | 3201; HOXA7 | 3204; HOXA9 | 3205; HOXB5 |
3215; HOXB7 | 3217; HOXC11 | 3227; HOXC4 | 3221; HOXC9 | 3225; HOXD3 | 3232; HSF4 |
3299; ID2 | 3398; IRF2BP2 | 359948; IRF6 | 3664; IRX1 | 79192; IRX4 | 50805; IRX5 | 10265;
JUN | 3725; KLF12 | 11278; KLF4 | 9314; KLF6 | 1316; LCOR | 84458; LDB1 | 8861; LMO1 |
4004; MAFF | 23764; MAZ | 4150; MBD1 | 4152; MEF2C | 4208; MEIS3 | 56917; MIER2 |
54531; MITF | 4286; MXD3 | 83463; MYB | 4602; MYBL1 | 4603; MYBL2 | 4605; MYC | 4609;
MZF1 | 7593; NAB1 | 4664; NANOS1 | 340719; NFE2 | 4778; NFIA | 4774; NFIL3 | 4783;
NFKB1 | 4790; NFKB2 | 4791; NFXL1 | 152518; NFYC | 4802; NKX1-2 | 390010; ONECUT3 |
390874; OVOL1 | 5017; PAX2 | 5076; PAX8 | 7849; PBX1 | 5087; PBX3 | 5090; PCBP3 | 54039;
PCGF2 | 7703; PIM2 | 11040; PKNOX1 | 5316; POU2F2 | 5452; POU4F1 | 5457; PPARGC1B |
133522; RNF10 | 9921; SALL1 | 6299; SIM1 | 6492; SIM2 | 6493; SOX7 | 83595; SPIB | 6689;
TFAP2C | 7022; TFCP2L1 | 29842; TP63 | 8626; TWIST1 | 7291; WT1 | 7490; ZBTB32 | 27033;
ZEB1 | 6935; ZNF367 | 195828;

TABLE 19A-35
Treg

FOXP3 | 50943; GPR83 | 10888; IKZF2 | 22807; IKZF4 | 64375;
LRRC32 | 2615; PENK | 5179; TNFRSF4 | 7293; TNFRSF9 | 3604;

TABLE 19A-36
WNT-Signaling

ADGRA2 | 25960; APCDD1 | 147495; DKK2 | 27123; FRAT2 | 23401;
FZD5 | 7855; FZD7 | 8324; ISLR | 3671; LZTS2 | 84445; RYK
| 6259;

TABLE 20
Cgnz1 Locus Genes
The human orthologs of the genes in Table 20 can be used as effective
biomarkers for classifying the LN disease state of the patient.

1700009P17Rik (Cfap126); Adamts4; Apoa2; Arhgap30; Atp1a2; Atp1a4; B4galt3; Casq1;
Cd244; Cd48; Cd84; Dedd; F11r; Fcer1g; Fcgr3; Fcgr4; Igsf8; Itln1; Kcnj10; Kcnj9; Klhdc9;
Ly9; Mpz; Ncstn; Ndufs2; Nhlh1; Nit1; Nr1i3; Pcp4l1; Pea15a; Pex19; Pfdn2; Ppox; Pvrl4;
Refbp2 (Alyref2); Sdhc; Slamf1; Slamf6; Slamf7; Tomm40l; Ufc1; Usf1; Usp21; Vangl2;
Wdr42a (Dcaf8);

TABLE 21
DEGs (in glomeruli and TI) from NZM2328 AGN mice
The human orthologs of the genes in Table 21 can be used as effective
biomarkers for classifying the LN disease state of the patient.

Glomerulus
Htr1b; Alox5ap; Gpr31b; Ace; Epha6; Ms4a6b; Peg3; Clec4a3; Ms4a4c; Siglece; Steap4;
Gm11172; Ms4a6d; Fyb; Itgam; Cyth4; Ms4a6c; Ms4a4a; Cfb; Pld4; Cd244; Lyl1; Postn; Irak3;
Atp2b4; Naip6; Myo1g; Sirpb1a; Cdon; Sirpb1a; Fmnl1; Clec4a1; Itgb2; Samsn1; Ptpn6;
AI607873; Hck; Tgfbi; Eid3; Myo1f; Xkr4; Gm14699; Treml4; Nlrp3; Pira1; Pirb; Lcp2;
Gm10198; Csf1r; Bcl6; Fgr; Cd22; Smpdl3b; Gm14548; Nfkbia; Themis2; Gm7609; Ikzf1;
Nfkbiz; H2-Ab1; Eno3; Gm5815; Fcgr4; Cybb; Tyrobp; Sla; Gsap; Iqgap2; Gm15448; Tnfaip8l2;
Gm25720; Gm11710; Ptma-ps1; Tnfaip3; Mir692-3; Tnfrsf1b; Naip5; Lilrb4a; Gm5150; Tbc1d8;
Ctsz; Gm15925; Pik3ap1; Bcl2a1a; Bcl2a1b; Lair1; Figf; Slc11a1; Plek; Psmb8; Ighv1-53; Pilra;
Adrbk2; Sepw1; Lpxn; Icosl; Cd274; Itgal; F10; Gm23722; Apbb1ip; Gm27864; Gm11709;
Nckap1l; Rassf4; Nrxn3; Il17ra; Ftl1; Arhgap30; Clec4n; Serpina3g; Itgax; Rac2; St8sia6; Ctss;
Icam1; Akr1b7; Lilr4b; Tubb2a-ps2; Sowahd; 6330407A03Rik; Ighv1-55; Gm15433; Lcp1;
Clec4b1; Ptprc; Tmem38b; Pglyrp1; Adgre4; Gm7592; Aifm3; Irf5; Pparg; Fcer1g; Gm15930;
Lilra5; Naip2; Pik3cg; Lpcat2; Plcg2; Pot1b; Clec2i; Pira6; Gm17097; Trem3; Xdh; Slfn8;
Dock2; Pqlc3; Il1b; Stap1; Ntrk3; C1qb; Igsf6; Rasgrp1; Igkv8-30; Cx3cr1; Coro1a; Cd9;
Coro1b; Gpr35; Irs2; Nek1; Pou2f2; 5430437J10Rik; AB124611; C3ar1; Tifab; Tnf; Cd74;
Ptk2b; Cd86; Adrb2; Rpsa; Vav1; H2-Eb1; Trpv2; Dok3; Mir29b-1; 5031414D18Rik; Zbp1; Rel;
Ncf4; Pstpip2; Arhgap15; Stk17b; Mt1; Mir692-2; Igha; Pyhin1; Cd44; Mir223; Cytip; B4galnt1;
Klkb1; Gadd45b; Clec12a; Gm4660; Gltscr2; Dusp2; Lilra6; Gm10693; Runx1; Cxcl13; Ighv1-
69; Ly86; Ikbke; Adgre1; Mir30d; St6galnac3; Mt2; Rplp0; Gm20004; Ighv1-11; Lst1; Rab3d;
Tmem 123; Serpine1; Cd180; Frmd3; Csf2rb; Lhfp; Lacc1; Cadm1; B4galt5; A630001G21Rik;
Ren1; Spi1; Fcgr3; Arrb2; Gimap3; Serpina3f; Cd48; Igkv3-2; Efhd2; Csf2rb2; LOC100861864;
Trib1; Tspo; Mvb12a; Mpeg1; Ighv1-70; Evi2a; Casp4; Cd300e; Ipo9; Pak1; Ighv1-62-1; Spn;
Casp12; Trf; C1qa; Dusp6; Lgals1; Gm22338; Gm19938; Cd300ld; Cd68; Cybrd1; Svil;
Mir7118; S100a6; Bin2; Mir30b; Slpi; Cry1; Fam129a; Cd84; RP24-113D21.1; Gm24272; Csf1;
Stk10; Fam111a; Ccl6; Gm11598; Gm20746; Tank; Rnf19b; Klhl32; Sh3bp2; Rnh1;
4833438C02Rik; Vrk1; Gm11710; Wdr83os; Pilrb1; Wfdc17; Gm24959; Ptafr; Cd300a; Gabrb1;
Apobec1; Psmb9; Slc46a3; Nfkbib; H2-Aa; Gm8730; Mir692-1; Cyp4f37; Gch1; Ctnnal1;
Gm4955; Ubash3b; H2-T22; Gm15453; Ighv1-5; Sp110; Sh3bgrl3; Enpep; I830077J02Rik;
Gm11034; Sele; Msr1; Ltb; Pla2g15; S100a9; Taok3; Clec2d; Mir6983; Tgif1; Gm13710; Neurl3;
Atox1; Slc15a3; Vcam1; Apoe; Camkk2; Slc9a9; Cyp4f16; Acp2; AF251705; Laptm5; Gm26951;
Ifitm2; Fermt3; Glcci1; Nrep; Ncam1; Gm17428; AW112010; Tubb2b; Sfxn3; Gm13015; Gngt2;
H2-M3; Hscb; Smtnl1; Pfn3; Pcgf5; Ifi204; Rplp0-ps1; Rasa4; Abcd1; Lctl; Gm20645; Mgat5b;
Tox; Tmem192; Ctsc; Adam22; Lyz2; Csf3r; Spata24; Serpina1b; H2-DMb1; Slfn2; Irf8; Setbp1;
Ms4a7; Ccnjl; Zyx; Prkcb; Alpk1; Cacna1c; H1fnt; Dock8; A530040E14Rik; Fcgrt; Hp; Gnai1;
Cfp; Gm26119; Cln3; Gba; Gm15590; Gpx4; Hmgn216; Hcls1; Fam49a; Htra2; Klhl6; Aif1;
Nfkb2; Fras1; Vmp1; Scel; Gm19765; Spats2l; Gm13846; Mir6995; Ncf2; Gm13063; Lrrc25;
Mir7070; Cyp1b1; Ccdc115; Celf2; Noct; Fam49b; Cd83; Rnf149; Cp; Ly9; Gm1153; Snx10;
Lyz1; Rmnd5a; Gm19422; Lrrc49; Gm23119; Abcb1a; Sirpa; Rad21; Gm20386; Serpine2; Emp3;
Rab29; Ccl19; Gm11594; Jam3; Dennd1c; Rasgrp4; Npl; Nadk; n-R5-8s1; Ptpre; Trim30a; C1qc;
Irak2; Sort1; Qsox1; Sp140; Gm19680; Pdia4; Rap1gap2; Vstm4; Epb41l5; Npas2; Rgs6; Selplg;
Gm2619; Rps8-ps4; Gm15538; Naaa; Ctsb; Krt80; Pnkd; Gm4242; Clec7a; Skap2; Gm11192;
Prkcd; Gm17402; Ier51; Stmn1-rs1; Arhgdib; Unc93b1; Sh3bgrl2; Gm17606; Gm6706;
Gm22637; Adcy7; Mnda; Pdlim1; Nt5c; Rps9; Stk26; Gm4799; Bola2; Ighm; Cxcl2; Gm5637;
Gmfg; Gm17111; Gm10115; Rnase6; Ccl19; Gm19474; Gm11360; Arl5c; Cx3cl1; Tlr2; Rps10-
ps1; Arhgap4; Selm; Rasa3; C130026I21Rik; Cpne7; Itgb3; Traf1; Ttc39b; Cd47; Cxcl1; Rpl12;
F2rl1; Nfam1; Gm7879; H2-Q5; Fam213b; Nr4a1; Gm9895; Sorcs1; Mob3b; Loxl2; Golim4;
Gm24098; Gm25381; Schip1; Cd52; Gm6085; Atp13a3; Tmem173; Rpl3-ps1; Dusp15; Cryga;
Dffb; Slc8b1; Ifngr2; Gas7; Chrac1; Tlr7; Gm11478; Gm22751; Mir30a; Thsd7a; Rarb; Ifitm1;
Lbx2; Ccl2; Gm25595; Oxsr1; Tuft1; Arhgap39; Tgfb1; Bmp3; Tspan4; C1ra; Krt7; Npm3;
AI662270; Arvcf; Gm22311; Plekho2; Car13; Arpc1b; Tmem219; Nfkbie; Parvg; Gm12626;
Hexa; Cklf; LOC100861894; Nrp2; Jchain; Gm9392; Pydc4; Gm12407; Gm15961; Lgalsl;
Mannr; Npr3; Plac8; Zfp229; Snora73b; Gm12443; Nuak1; Lgals1-ps1; Rpl18-ps1; Edn1; P3h4;
B3galt2; Gm9625; Gm10516; Tap1; Dctd; Fxyd6; Gm4919; Gm7079; Gm25992; Nebl; Ppfibp2;
Rpl18a; Nsun3; Man2b1; Rps6ka1; Mocos; Gm20627; Abhd12; Clock; E330009J07Rik; Myh11;
Gm15358; Ccl19; Tlr1; Gm20463; Grm4; Parl; Lyn; Hn1; Nrd1; Gm26510; Dclre1c; Rab3il1;
Krtcap2; Dnml; Enox1; Sp110; Gm13401; Col18a1; Blmh; Mir1962; Gnb2l1; Lyst; Mir6915;
Gm1587; Slc15a4; Pycard; Zc3h6; Rbm3; Lsp1; BC018473; Jam2; Gm17541; Mir8103;
Gm14944; Inpp5d; Tbc1d9; Pnpla7; Sptb; Creg2; 4631405J19Rik; H2-T10; Tppp; Cd37; H13;
Cyba; Syde2; Dapp1; Tmem51; Tbpl1; Tmsb4x; AI413582; Spsb2; Gm11747; D930015E06Rik;
Gm11425; AA666905; Mir669f; Fam96b; Emc9; Nrip2; Gm5899; Ppp1r14b; Cap1;
4921504A21Rik; Dynll2; Tinf2; Gm15675; Vti1a; Nup107; Nol3; Ppp1r14d; Tgm2; Ifi2712a;
Rab8b; Rcor3; LOC100861648; Bmp2; H19; Gm13681; Gm15772; Gm11465; Gm20186;
Tm6sf1; Ighv5-9; Gm5866; Ighv1-56; Slc25a43; Ifitm3; A130040M12Rik; Ar; Rpsa-ps10;
Camk2d; Ahr; Zfp703; Hrc; Snord123; Gm10181; Mir7221; Tigd2; Snord13; Traj57; Cd248;
Gm20620; Dpp4; Capg; Wdfy4; Gm15539; Gm11724; Hfe; Gm8709; Grn; H2-Ea-ps; Nr1h3;
Irak4; Acot9; Hmox1; 1600027J07Rik; Tceb2; Lsm14b; Scimp; Syk; Gm11361; Gm23694;
Hmga2-ps1; S100a4; Gats13; Mir24-2; Gm27722; Gm9525; Rpl28-ps1; Sh3bp1; Psmb10; Il1rl2;
Gm12097; Ctsl; Gm6607; Hipk3; Gm10855; Rgs19; Gm12151; Syt9; Gm14165; Nrros;
Zmynd15; Gusb; Pop1; Gm10051; Lsm4; Brms1l; Zfp36; Slc18a2; Ssh2; Psmg4; Slamf9; Pltp;
Il2rg; Ka1rn; Rpsa-ps9; Gm15755; Gm11539; Snord13; Rhog; Nars2; Arsi; Commd5; Snord65;
Mir8102; Rpl32; Mir3472; Mir3472; Gm13192; Zc3h14; Gm25145; Tek; Ear1; Lncpint; H2-L;
Cdk4; Galc; Ccr2; Otub1; 11-Sep; Pfn1; Pydc3; Ap1s2; Cd36; Arhgap9; Tnip1; Cacna2d2;
Gm10059; Chil1; Tspan5; D230017M19Rik; Rps10; Trpt1; Gm20120; BC031361; Gm536;
Gm19279; Ighv1-14; Gm6291; Gm9843; Nr1d1; Micall2; Rps29; Pisd-ps3; Trav9d-2; Arpc4;
Gm14323; Nfkbid; Gm25357; Ppp1r14a; Lrrc1; Gm24544; Anxa4; Frem2;
Tubulointerstitium (TI)
Ms4a6c; Gm10720; Gm11168; Gm17535; Gm10715; Gm10717; Igha; Itga6; Gm10719;
Gm10718; Gm10721; Igkv8-30; Kl; Xdh; Calb1; Gm10800; Gm21738; Jchain; Rel; Cyp4b1;
Cyp2a4; Ccl19; Cml3; Cyp2a5; Ccl19; ST7-OT4_3; Gm23375; Gm13785; Gm12407; Dusp9;
Gm6788; Npnt; Ccl19-ps1; Gm13416; Adamts6; Speer8-ps1; Cyp2d12; Cybb; Gm4482;
Gm23374; Snap91; Zc3hav1l; Gpr31b; Fgg; Ccr2; Actg-ps1; Slc35g1; Itgax; Lilr4b; Serpinh1;
Bcl2a1a; Cyp4a12a; AI607873; Speer4cos; Fam163a; 2310067E19Rik; Tyw3; Myo1g; Sp4;
Gm8399; 9530091C08Rik; Laptm5; Nfkbia; Acmsd; Lcp1; Irs1; Lamc1; Cyp2e1; H2-Eb1; Ccl19;
Mirt2; Gm14699; Ppard; Fabp1; Xpnpep2; Gm12079; Coch; Arvcf; Gm14778; Tmem254b;
LOC100861647; Gm21847; Igkv1-110; Dhtkd1; Gm6382; Kit; Gats13; Cyp4b1-ps1; Igkv12-46;
Ccl19; Dppa5a; Gm10737; Sparc; Ptprc; C1qb; Gm11710; Sult5a1; Foxq1; Cyp4b1-ps2; Ly6k;
Lncpint; Pld4; Ntn4; Etv1; 9530026P05Rik; Gm21968; Gm10592; 4930466F19RIK; Col4a3;
Mir27a; Gm7609; Nt5e; H2-Ab1; Cx3cr1; Fyb; Nupr1; Chst15; Car12; Mthfr; Aqp6; Col4a2;
Scgb1b19; Gm2810; Fam184a; LOC100861804; Klhl42; H2-Aa; Slc38a4; Pdhal; Igkv1-117;
1810063I02Rik; Slc25a19; Dusp18; Tk1; Bcl2alb; Mirlet7a-1; Ccl19; Acsm2; Gm2444;
Ankrd13c; Anp32-ps; Cyp4a12b; Tex19.2; Mir598; Igkv3-2; Zc3hav1; Gm5943; Nid2; Adgre5;
Hck; Aldh1a1; Coro1a; Slc16a1; 2700046G09Rik; Akr1c19; Gm2115; Gm22121; Gm10658;
Slc22a12; Ighv1-34; Tmem254b; Srsf11; Macrod2; Gm15660; Slco3a1; Gm5537; Tmem61;
Tmem254b; S100g; Gm11814; Dazap2; Gm15146; Calu; Cd3e; Gm17181; Dbp; Gm5510;
Stk381; Rgs5; Htra4; Lilrb4a; Lrp2; Nr1d1; Sel1l3; Hels1; Mir7211; Tfrc; Hist2h2be; Camk1g;
Per3; Mpeg1; Gm12651; Slc11a1; Gm10801; Jam3; Snhg11; Ighv1-15; Gm14604; Atp6v1d;
Gm25896; Mfsd6; Gm13782; Prdx3; Gm15724; Figf; Dpp4; Mthfd11; H2-DMa; Gm23455;
2610016A17Rik; Nckap1l; Gm9308; Pira6; Gm19852; Gm26134; Bcl6b; Ppara; Ankrd33b;
C130026I21Rik; Pdlim3; Gm26265; Tnfsf13b; Ncf2; Adralb; Upf3b; Gm19767; Efhd2; Mir34b;
A130040M12Rik; Dock2; Mvp; Gm11034; Arv1; Gm15573; Tspan17; Bglap2;
A130040M12Rik; Gltscr2; Plk1; Gm26219; Ripk4; Vps4a; Gm22602; Cerk; Gimap3; Gm24931;
Rapsn; Bglap3; Fxyd5; Gm26497; Gm10600; Gria3; Cc15; 1-Mar; H2-DMb2; Rdh7; Lcp2; Ighv1-
23; 1190007I07Rik; Sp110; Stradb; LOC100861887; Mia; Cpeb4; Vsir; Lonp2; Aplnr; Cadm1;
Gtf3c3; Chdh; Cpb2; Axl; Calcr; Rin2; Nfib; Hsd3b5; Cry2; Gm27552; Gm27844; Fbxl16;
Ccdc141; Ireb2; 9130008F23Rik; Acsl1; Gm26881; Tmem89; Endou; Gm13502; Mir6937;
Gm27011; 1700056E22Rik; Sla; Gm11709; Gm14734; Gm14654; Gm26217; Lyz2; Tagap;
Gper1; Slc25a15;

TABLE 22
DEGs (in glomeruli and TI) from R27 mice
The human orthologs of the genes in Table 22 can be used as effective
biomarkers for classifying the LN disease state of the patient.

Glomerulus
Htr1b; Postn; Serpine2; Dusp6; Svep1; Gpc3; Slfn3; Lrp1; Chst15; H2-Q8; LOC100862212;
Tbcld9; Uchl1; Psap; Tox; Cls1; Thbs1; H2-K1; H2-Q5; H2-Ab1; Cp; H2-L; Zyx; Cep83;
Trp53i11; Akap12; Cd74; Alox5ap; mt-Te; Angpt2; Apln; Mmp11; 4930453N24Rik; Cxcl13;
Ngf; Bcl6; Gm5866; Csrp1; Pla2g15; Gm6485; Mir30b; Mt2; H2-T22; Epha6; Tnc; Cdk6; P2ry1;
Fhod1; Lmf2; Fam160a1; Nxpe3; Capzb; Nrxn3; Gabrb1; Mir1906-1; Zfp654; Pltp; C1s2;
Zfp125; Pdgfrb; Ndn; Tgfb1; Slc23a2; Xdh; Gm17530; Sirpa; Ergic1; Cd9; Cxx1b; Bre; Pld4;
Cldn34c1; C3; St5; Gm15079; Osmr; Jag1; Naaa; Sema6b; Smad1; Icosl; Dock8; Mcam;
Tmed10; Gm22755; Gm14584; Chml; Trf; Pisd; Gm13669; Sorcs1; Bmp2; Ret; Mir29b-1; Ank;
H2-T10; Adm; Actb; Pisd-ps3; Agtr1a; Ypel2; Adamts1; Arhgap20; Gm6180; Gm10254;
Gm6821; Aldh1a2; Cacng7; Otub1; Pisd-ps1; Gpc6; Rhbdd1; Zcchc11; Schip1; Ntrk3; H2-Eb1;
Dchs1; Ttc39b; Zfp326; Nebl; Tnfaip1; Gufl; Cdkn1a; Tmem173; Cd9-ps; Tnfrsf10b; Gpcpd1;
Gm10778; Grn; Cyp26a1; LOC100862309; Fam179b; Slc9a1; Figf; Gm19868; Col12a1; F2r;
Tnfaip812; Cacnala; Gm13749; Reck; Csf1; Csnk1g3; Ogfrl1; Dnm1; Gm7609; C1rb; Gramd4;
Xkr4; Hspg2; H6pd; Etv1; Gm5931; Tpm1; Peg3; Dkk2; Vcl; Car13; Ajuba; Esam; Mir30d;
Aste1; Gpihbp1; Zfp329; H2-Q7; Micall2; Ahnak; Mtcp1; Zfp945; Cyth4; Lrrk1; Vcpip1;
Epm2aip1; Hcls1; Gm266; 3110052M02Rik; Fosl2; Ttc14; Rfwd2; Igha; Gm10778; Lef1;
Gm4024; Bmpr1b; Swap70; Snhg20; Dpagt1; Sh3bp5; C1ra; Gpr31b; Gm20386; Actn1; Atg3;
Axl; Cygb; St3gal5; Cald1; Cdkn2aipnl; Ctsb; Ace; Gna12; Fam204a; Man2a2; Lrrcc1; Rcn3;
Slc6a4; Adat3; Vav2; Clk1; Sost; Wdr91; Cd248; Crim1; Ch25h; Flnb; Akr1b7; N4bp3; Sidt2;
LOC100862325; Plxnd1; Mirlet7d; Mir694; AI607873; Mical2; Ccnd1; Ppfibp1; Ddost;
Arhgap12; Ly6e; Mysm1; Sec24d; S1pr2; Kit; Nuak2; Cgrrf1; Plekhm1; Pisd-ps2; Plbd2; Sclt1;
Pvrl3; Kcnq5; Klhl6; Gns; LOC100861810; Toporsos; Gm14639; Plxna2; Plxna1;
B630005N14Rik; Jam2; Actg1; Zfp40; Svip; Dcun1d5; Mir99a; Fabp4; Man1c1;
E230016M11Rik; Unc93b1; Abhd2; Gm10499; Csflr; Ppt1; LOC100862227; Gm15433; Rftn1;
Ctgf; 4930522L14Rik; Nt5dc2; Igip; Nrp2; Ror1; Lhfp; Cyth3; Abcg1; Rassf4; Adrbk1;
Tmem184b; Tcea1; Ranbp6; Rela; Adamts4; Mgp; Adam22; Ets1; Gusb; Npr2; Ifi44; Celf2;
Wasf3; Efcab2; Mir7026; Stab1; Taok3; Fam49a; Kctd10; Gm14541; Slc35d1; Extl3; Fam102b;
Paip1; Gm10509; Wdtc1; Cox20; Gt(ROSA)26Sor; Pttg1ip; Zfp182; Tmeff2; Pqlc3; Mfge8;
G6pdx; Man2b2; Col4a2; Stard13; Nid2; Zfp747; Vopp1; Plekho2; 4-Sep; Fkbp10; Flna; H2-K2;
Slco3a1; Heyl; Arrb1; Myl9; Zfp935; Sfxn3; Mocos; Sh3bp2; Cap1; Fh12; Dpf2; Plp2; Mir6539;
Anxa3; Ptpn9; Gm22067; Esco1; Zfp882; Klf6; P3h3; Fzd8; A530016L24Rik; Fam129a; Prex1;
Nid1; Mkl1; Paqr6; Tnfrsf1a; Sqstm1; Bst1; Gm12647; Scel; Rab8b; Trpt1; Rnf144a; Nedd9;
Cx3cl1; Acsf2; Mamld1; Sh3bp1; Atp6ap1; Naip6; Gm15925; Gm5225; Plod3; 5430416O09Rik;
Gm12276; Themis2; Cdh3; Ighv1-5; Tbc1d8; Cxx1a; Sell; Lztfl1; Gm10337; Phf6; Erich4;
Txlng; Wdr83os; Igkv1-110; H2-DMb2; Amd2; Hexa; Gm12091; Zswim1; Pemt; Gm7592;
Gm14308; Mpeg1; Slc12a4; H2-Q4; Gm11709; 2610002M06Rik; Gtf2h1; Yipf4; Zmynd15;
Gm15784; Dcaf7; Tubb5; Rce1; Rtn1; Trim16; LOC100862055; Tbc1d2b; Cmpk1; Arhgdia;
Psen2; Terf2ip; Stat3; H2-M3; Stk26; Tcf12; Grap; Smpd4; Akna; Pcdh12; Pexl3; Gm4130; Irf8;
Synj2bp; Tek; Itgb2; LOC100861712; Gm5046; Wdfy4; Sgsh; Phf20l1; Rnpc3; Eefsec; 9-Sep;
Mrpl35; Scimp; Sumo3; Ifngr1; Mmrn2; Pyurf; Nfkbiz; N4bp2; Mir6924; Tns3; Sncg; Tmem110;
1700092M07Rik; Cped1; Il6st; Frmd3; Ampd3; Cfl1; Gm16265; Mrpl32; Xpo6; Ciita; Serpine1;
Bgn; Gm6054; Gdpd2; Mmp2; Syp; Plekha2; Cxcr4; Tpm2; Rpl8; Gm12013; Mbtd1; Spin4;
Etv6; Tgfbi; Gm19601; Fbn1; LOC547349; Hmgn5; Setbp1; Mir181b-2; Ackr3; Lama2; Plcg1;
Zfp809; Rapgef5; Gm2573; Wwox; Dtx4; Gm20939; Usp22; Zcchc14; Zfp59; Dhx36; Adam12;
Slc37a1; Caap1; Lilrb4a; Zfp457; Utp14b; Plvap; Zfp597; Plppr2; Cand2; Adgrg1; Fat1; Pdcl;
H2-DMb1; Scn4b; Gm9958; Hdac2; Ercc1; Gm20257; Myd88; Jam3; Baiap3; Fstl1; Suz12;
Sh2d3c; Arglu1; Grk5; Snord98; Kalrn; Gm22105; Gm13487; Syt11; Utp18; Ust; Pcdhgb1;
Nckap1l; Itga9; Igkv1-117; Piral; Cutc; Col4a1; Pkm; Capn7; Dlx5; Ppwd1; Treml4;
2310022B05Rik; Rfk; Mif4gd; Fscn1; Rmnd5a; Gm6695; Ttll12; Nkain4; Qpctl; Daglb; Gm5637;
Myo1g; Arhgap6; Gm14699; Hid1; Pear1; Btd; Capg; Pip5klc; Gm6517; Cdc73; Gm13140;
Zfp273; Plcl1; Atp11a; Mrps30; Cd93; Fcgr4; Ncsl; 4931440F15Rik; Lixll; Pld3; Zfp518a;
Rmdn1; A730008H23Rik; Tafld; Plekhm3; Atp5l; Taco1; Rnf138; Steap3; Cfb; Laptm5;
Nkiras1; Pstk; Gm16488; Lrrc32; Luc7l2; Cyp1b1; Lgals3bp; Gm13294; Dusp1; Ccdc173; Ubb;
Pld2; Ube2v1; Pank1; Gm26407; Sfpq; Cpt1c; Polr2k; Ubl7; Zfp942; Gm12492; C4b; Ighv1-12;
Pdcd4; H2-Q2; Camk2g; Cdh5; Notch2; Cst3; C1galt1; Spsb1; Itgax; Gm4875; Gm26951; Swt1;
Gm11940; Adamts5; Pnpt1; Ttll1; Zfp758; Gm7353; Zranb1; LOC100862198; Cpne4; Prkch;
Tbc1d19; Rcor3; Trp53; Slc11a1; Gmps; Dpysl3; Stxbp3; Hdac9; Pet100; Gm11710; Napa;
Wfs1; Gm22578; Nup98; Rpa1; Birc3; Aoc3; Cnn2; Lamc1; Zfp386; LOC100862331;
5830428H23Rik; Fam188a; Med12; Tlk2; Tnip1; Gm26944; Pcsk6; Gm16356; Arfgef3; Rhoj;
Laptm4b; Ccdc181; Mt1; Slirp; Gm12619; Naip2; Myo1f; Gm14567; Msn; Ndufaf5; Vps28;
Ccdc90b; Dbn1; Lcorl; Itga5; Gm17366; Maea; Glmn; Amd-ps3; Ppm1l; Tbc1d8b; Gm15269;
Zfp101; Gja5; Plxnb2; Erbb4; Adam15; Smt3h1-ps; C1qb; Tmem26; LOC100861682; Gcc2;
Nav2; l7Rn6; Vps33b; Tiparp; Gm21897; Snhg5; Sorbs2; Phfl4; Zfpm1; Gng7; Pelp1; Myh10;
Pus3; Tmem47; Spata13; Hist2h4; Iqgap1; Zfp260; A230050P20Rik; Ikzf2; H2-DMa; Qsox1;
Smarca5; Lat; Emc1; Thap1; 1190002N15Rik; Gm9025; Piezo1; Hs1bp3; Il4ra; Phxr4; Clstn1;
Gm14698; Rsrc2; Nudt10; Zfp638; Ednra; Gm20053; Zfp281; Ctbp1; Tgfbr2; Lmcd1; Kbtbd11;
Gm12723; Ap2b1; Gm6505; Fbln7; Ccbe1; Cln6; Eng; Gpm6b; Fam25c; Clk4; Slfn8; Gm19941;
Ctss; 4732440D04Rik; Ptafr; Gm25278; Coro1c; Gm1974; Tapbp; Mir34a; Wrb; Rasl11a; Lilr4b;
Atp10a; Ighv1-55; Ctc1; Timm9; Rev31; 1810030O07Rik; Des; Dock7; Flt4; Pitpnm1; H2-Aa;
Zdhhc1; Thoc2; Conc; Pparg; Mb21d2; 2300009A05Rik; Irak3; Sema3f; Cacnb3; Ikzf4; Soga1;
Ptges3; E230008O15Rik; G2e3; Gm5423; Loxl1; Slc35a4; Gimap3; Stom; Orc4; Icam1; Tspan9;
Pcnxl3; Pcfl1; Slc9a8; Olfr1372-ps1; Msh3; Larp1b; Etv5; Gm21956; Sepw1; Snapc3; Rps2-ps9;
Psmb8; Eif6; Gm7870; Zfp708; Gm21411; Smek1; Tgfb3; Ankrd1; Serpinb1a; Zfp426; Ighv2-2;
Cers5; Osgepl1; Nr4a1; Mapk3; Fam92a; Gm12727; Zfp846; Smc5; Myc; Tsc2; Col6a3; Svil;
Gnrh1; Trp53-ps; Rtn4rl1; Rock1; Map4k3; Gm14431; Gm14431; Gm14421; Gm11966; N4bp1;
Ccndbp1; Zfp759; Pkd1; Clec4a3; Gm10491; Mgat4b; Tnfrsfl2a; 1700017B05Rik; F2rl1; Eid3;
Slc10a6; Gm20300; Fyb; Zdhhc18; Vill; Gorasp2; Slc6a6; Smarcd1; Gm14108; Gm1848;
Ccdc102a; Rbm12; Fmr1; Fam53b; Paxbp1; Gm5454; Gm14431; Scarb1; Gm4242; Gm26184;
2210404O09Rik; Mir219a-1; Creg2; Cdc42bpb; Rad54l2; Cntln; Abcd1; BC024416; Gm20174;
Smim10l1; Nacc1; Itgb3; Samd4; Pygm; B3galt2; BC002059; Irak2; Adam9; Gm4540; Ebag9;
Bin1; Gm15067; Gm17138; Snora24; Plod2; Ccdc50-ps; Ubxn2b; Ndufab1; Tlr2;
C920025E04Rik; Lama5; St3gal1; H2-Ea-ps; Snord123; Rpl39; Gprc5a; Khdrbs3; Ighm; Il17rd;
Hsf2; Ttc28; Dnttip2; Slc44a2; Hck; Gpr153; Cul7; Ankra2; Amph; Gm22940; Glrx3; Gm1840;
Zfp85; Cers4; Lipo4; Tnfaip2; Cd82; Rcor3; Rhbdf1; Igkv2-137; Bcl2a1a; Gm22507; Gm11949;
Znhit3; Tusc5; Chpt1; Gm24715; Stk10; Gm8648; Malsu1; Myo1b; Prkd2; Gm24876; Gm25969;
Gm19899; Tspan31; Pgf; Gm26141; 1110059G10Rik; Stard3nl; 1810022K09Rik; Gm6542;
Lcp1; Gm25654; Gm13786; Nmt1; Gm13691; Gm14548; S1pr3; Vat1; Snx3; Clptm1l;
Gm13319; Tmem256; Esm1; Crtc3; Mir6989; Ext2; Stc2; Gnpnat1; Gm25636; Leprotl1; Otud6b;
Snx17; Mir677; Ptpn23; Rasa3; Psen1; Luc7l3; Sigmar1; Mir721; Ramp3; Npr1; Gm11331;
Leprot; Gm15562; Cd40; Tor4a; Akt2-ps; Shroom4; Mtss1l; Dusp5; Slc38a4; 6030408B16Rik;
Pde8b; Dcun1d1; Trappc9; Tut1; Gm24145; Stim1; Ssb; Spcs1; Phf20-ps; Mir680-2; Pcmtd1;
Pik3r1; LOC100861648; Zfp960; Ino80c; Impad1; Gm15065; Rars2; Spon1; 1810010H24Rik;
Pml; Gm22094; Phlda3; Lypla2; Stmn2; Rbm39; Zfp131; Plppr5; Gm15446; Tpm3-rs7; Robo4;
Edn1; Ndufb4; Asph; Gm12112; Tpst2; Ssbp1; Tmem43; Ets2; Ercc2; Pigs; Smim15; Elk3;
Ndst1; Zfp948; Pkig; Cask; Gm7030; Gm2004; Nlrc5; Arhgef2; Apbb2; Prkca; Gabra4;
Gm15753; Gm10855; Gm16284; Ap3dl; Cmtr2; Gm24930; Sbk1; 9330159M07Rik;
2610035D17Rik; Gm11451; Gm17576; Nol3; Zfp729a; Hacd1; Homer1; Bmper; Fam126b;
Plppr2; Gm12840; Gm11009; Gm14401; Zfp958; Nol6; Fam135a; Rnaseh2c; Parg; Itsn1;
Gm15972; Gm15861; Gm12669; Gm17546; LOC100862189; Gm15519; Bcl2a1b; Gm6206;
Bin3; Gm15378; Ly96; Prkrir; Glt28d2; Cybb; Fgr; Sema4b; Atr; Abr; Gm14288; Sumo1;
Gm19372; Ephb1; Adprh; Bcr; Gpr27; Oxsm; Gm14393; Cirh1a; Smc6; Gm24630; Gm14400;
Usp10; Lactb; Gdpd5; Orc6; Gm4353; Nudt7; Efemp2; Gemin5; Trak1; Zfp322a; Mcrs1; Nek1;
Psmc6; Asap1; Trbd1; Tmem63a; Usp16; Ist1; Vstm4; Zfp738; Ube2g2; Dld; Scap; Zdhhc7;
Ntrk2; Tmem206; Setd8; Sbno2; LOC100861785; Sema4c; LOC100861916; Chst11; Brcc3;
Trib1; Bmp1; Hmga1-rs1; Anxa11; Tle1; Vps29; Gm16199; Gm3150; Lrp5; Rcc2; Cxcl16;
Zfp932; Rhod; Igkv8-30; Atp5j; Terfl; Gng5; LOC100862018; Oaz1-ps; Gm27690; Tst; Adrbk2;
Med21; Abi3; Mrps36; Pde7b; Nadk; Gm20305; 5-Mar; Rps6ka1; Ssfa2; Crry-ps; Ehbp1l1;
Igfbp2; Gata3; Eif2b2; Gm6863; Gm5612; P2rx1; C330013J21Rik; Dll4; Deb1; Gprc5b;
Mir1954; Igsf6; Plekhg2; mt-Tq; AA474408; Fbxw4; Acyp2; H2af-ps; Nr3c2; Kirrel; Enthd2;
4921504A21Rik; Slc26a10; Adora2a; Acox2; P2ry6; Tspan33; Ighv1-56; Csrp2; Zfp820; Cnot6l;
Gm11008; Xpc; Ppp2r3c; Mettl5; Gm11914; Nutf2-ps2; Galns; Arsa; Gm14000; Ighv1-14; Rbks;
Mrps21; Gm15448; Gm11405; Gng2; Rnps1; Tyrobp; Rgs9; Polr2k-ps; Gm14416; Gm20775;
Hilpda; Gm11034; Tpcn1; Mterf1b; Gm2399; Gars; Mir6929; Hcfc1; Vps50; Cnot7; Jchain;
Gm17296; Olfr1055; Ints3; Nomo1; Cnnm3; Tmem120b; Ighv1-62-1; Trp53inp1; Tnfrsf1b;
Taf15; Ocel1; Gm22829; Plxdc1; Zfp229; Anks1b; Plau; Gaa; Add2; Txnip; Ptger4; Zfp617;
Gm20199; Vbp1; Gm2260; Gm2026; Gm16581; Ppp1r18; Rb1cc1; Dag1; Serinc3; Jade2;
Map3k3; Cdh6; Dapk2; Gm26533; Znfx1; Csprs; Ube2v2; Zfp442; Cwf1912; H2-Ke6; Tctn1;
Prrc2b; Acp2; Gm1826; Dynlt3; Myh11; Pde5a; Cyr61; Pnpla2; Ssrp1; Csnk1d; Tmem161b;
Tpm3-rs2; Ccar2; Lncpint; Kti12; Plek; Furin; Cldn5; Myl12a; Papolg; Gm14308; Zfp280d;
Ighv1-31; Prr23a2; Hnrnpll; Gm12715; Gm6712; Zfp87; Ehd2; Ighv1-73; Specc1l; Gm11644;
Lrrc3b; Gm1821; Bbs7; Gm13810; Fam199x; Fkbp3; Timm8a1; Gm26166; Ube2v1; Sp3; Vac14;
Hist1h3e; Gm14409; Ggh; Atp2a3; Rhbdf2; Impa1; Pofut2; Zfp60; Apoe; Fndc3b; Sik1; Nos2;
Fxyd1; Gm4853; Mir322; Flrt1; Zdhhc4; Msl2; Ogg1; Cbfa2t2-ps1; Yae1d1; Pcm1; Dkk3;
Ccnb1ip1; Fndc4; Gm10012; Rpl26; Zfp97; Srek1; Tcf25; Gm14420; Il1r1; Pwp2; Wwp2;
Dpysl2; Nudt7; Eif2ak2; LOC100861882; Abca1; Prpf8; Gadd45b; Trim12c; Tcf4; Pirb; Irf4;
Anxa5; Gm14431; LOC100862206; Slc15a4; Gm6729; Gm12484; Vps13a; Dzip3; Parp16;
Gm19514; Slc15a3; Gpi1; Slc48a1; Plk3; Tet2; Myo9a; Chuk; Tbc1d22a; Gm15148; Arhgap5;
Ncstn; Gm15327; Sh2b3; Dlg1; Gm19474; Fam120aos; Mocs2; Asun; S1pr1; Ece1; Afap1l2;
Kras; Cacna2d1; Dr1; Gm14408; Zmiz2; Mast3; Ythdc1; Gm15846; Lyrm4; Gm21962;
Casp8ap2; Snord104; Nrbp2; Mir5108; Slc41a2; Eml2; Gipc3; Gm5921; Zc2hc1c; Atp13a3;
Kif21b; Rnf41; Gm13242; Gm26779; Tmem63b; Tm2d1; Rasa1; Gm14405; Cyb561a3; Pyroxd1;
Rbm26; Snord72; Arhgef17; Timp2; Gm26771; Exosc7; Fam81a; Zadh2; Eif1ax; Arhgap27os1;
Slc16a4; Gm2619; Gm5070; LOC100862021; Gm8988; Platr25; Rad9a; Gm7160; Ncf4;
Gm8532; Fermt3; Rora; Pkn2; Cyp2d12; Zfp931; Scn1b; Dnajc19; Ints6; Igdcc3;
2010315B03Rik; Etohi1; Krt40; Ap1ar; Psme4; Cfap97; Fam234a; Usp1; Mir3470a; Acot4;
Pianp; Zfp62; Cpne7; Ube3b; Snap23; Traf3; Mis18a; Gnai1; D930016D06Rik; Ccdc88a;
Arhgap17; Gnai2; Cml1; Zfp36; Cyb5r4; Gm12420; Gm6686; Tubg1; Npc1; Plac9b; Arhgef10l;
Slc25a46; Suv39h1; Gm3244; Cdc42ep3; Tap1; Gm10136; Gm19486; Ddx39b; Vps11; Rpl39-ps;
Gm9922; Slc33a1; Gm13597; Twsg1; Chmp3; Ccdc191; ND6; Qser1; Tnfaip3; Cptp; Magoh;
Myo9b; Tln1; Arl5a; Arhgef6; Gm14392; Rac1; Ubr4; Gm25660; Gm14308; Vstm2b; Efcab7;
Angel2; Mtmr14; Gm14436; Crkl; Zbtb6; Gm14933; Sms; Rabgef1; Gm26826; Mink1; Clip2;
Mtrf1l; Zfp36l1-ps; Gm25799; Ces2c; Celf4; Gm10808; Gm9909; Rapgef3; Nr2f2; Serping1;
Pibf1; Ap3m2; 3110070M22Rik; Mfng; Tpk1; Ambra1; LOC100505224; Gm14391; Gpbp1;
Pspc1; Gm6741; 5730408K05Rik; Runx3; Rrm1; Rbm12b2; Cyp51; Trappc10; Gm2274; Lsm3;
Ints9; Gm14405; Ctdp1; Mpzl1; Tmem167; Pdcd6ip; Epha2; Gm23462; Spry4; Gnptab; Pex2;
Mecom; Ptcd3; Gdf10; Ldlrap1; Hoxb3os; Rbbp6; Dcaf6; Sla; Atpla3; Arf1; Nisch; Zhx1;
Zfp458; Gm20290; Smim8; Runx1t1; Pdgfra; Gm14308; Kank2; Sema3g; Gm13797; Gkap1;
Fam134c; Gm22639; Angptl2; Hook3; Nupr1; Gm13342; Gm4459; Map4k1; Gm20186; Efemp1;
Hist3h2ba; Ube2i; 2210418O10Rik; Mbnl3; Gm14308; Zfp119a; Klhl28; Uevld; Ap3m1-ps;
Tgif1; Prdx2; Mmab; Gm15129; Clec4b1; Atf6; Gm13204; Gm14288; Gm10974; Emd; Mcts2;
Tex9; Lsp1; Plin3; Gm22875; Pon3; Dsel; Irf5; Mup-ps26; Apobec1; AU041133;
LOC100861649; Chmp4c; Hoxa10; Phc2; Yes1; Tnpo2; Gm13695; Orc3; Cln5;
D630045M09Rik; Myh9; Atp6v0b; Gm23413; Tmf1; Tug1; Amd2; Gm12502; Ggcx; Cacul1;
Cep295; Cdk2; Acbd5; Uba1; Cd14; Ednrb; Ifnar2; Gm9222; Camkk2; Fastkd2; Ssh1; Dhx37;
Gm13131; 4930430F08Rik; Gm10193; Gm14422; Nck1; Gm21954; Mir8100; Gm13698;
Gm13693; Gm13696; Gm13694; Gm13697; Hist1h2an; Tsc22d2; Gypc; Gnb2; Smek2; Zfp292;
Lipo1; Wsb2; Gm24081; Pdlim1; Ccdc82; Sowahd; Cstf3; Gm10222; Gm12291; Copg1; Crebrf;
Rergl; 2810474O19Rik; Tle3; Egr1; Fzd4; Bop1; Nav1; Purb; Proser3; Hacl1; Nipbl; Atg4b;
Gm14894; Nop2; Plac9b; Evi5; Crip2; Styx; Vps37a; Exoc8; Lrrc17; Dctn1; Mark2; Echdc1;
Trip4; Ube3a; Prelid2; Ankrd27; Ano6; Rhob; Gm16124; Btg2; Bace2; Ucp2; Gm26135; Tbl1x;
Jak3; Kansl1l; Gm15770; Ppm1m; Pla2r1; Fos; Ift74; 5730455P16Rik; Cd274; Sgsm3; Mboat1;
Trmt2b; Zfp35; Mapk12; Slc51b; Adgre4; Nufip2; Zfp280c; Gm6361; Gm3873; BC003331;
Samd4b; Ticam1; Runx2; Gm18025; Pecam1; Gm10420; Ids; Ache; Gm16357; Lman2; Ice2;
Patl1; Gm26853; Emp2; Gm22548; Entpd1; Tom1l2; Gm13936; Tceal8; Gm17608; Snhg6; Rpn2;
Epas1; Map1lc3b; Tes3-ps; Tep1; Syvn1; Snx9; Psip1; Gm24149; Igsf3; Hace1; Alkbh3; Nucb1;
Socs6; Gadd45g; Ociad2; Gm20691; Gm14387; ND3; Ppp4r2; Gm16363; Hmox1; Anxa6;
Marcksl1; Tmem98; Vasp; Tmem41a; Ufsp2; Kif3c; Cd22; Hyi; Flrt3; Nfkbie; Gm24592; Plcb3;
Gm10139; Rdh19; Hist1h2al; Fundc2; Phf2os1; R3hcc1l; Dnajc2; Mmp14; Tmem87b; Cops2;
Htatsf1; Sap25;
Tubulointerstitium (TI)
Cyp2d12; Kit; Gm13487; Plekhb1; Figf; Cd74; Clenka; Gm13212; Speer8-ps1; Paqr5; Slc12a3;
Gm8116; Aldh1a1; Gtpbp1; Cnnm2; Plxnb2; Napa; Ptk2b; Rela; Gm10139; Rnf186; Cyp4b1;
Ly6e; Hcfc1; Cluh; Usp22; Grn; Tmem94; Sorl1; Gm14392; Cpt1a; Zmiz1; Bckdk; Wdtc1; Insr;
BC026600; Ndrg2; Gm27742; Sirt2; Smc4; Efhd1; Syngr2; Akt2-ps; Gprc5c; Xab2; Cep851;
Sdc4; Zdhhc1; Mrpl32; Gm9755; Gm15664; Sirpa; Serinc2; Cyp4b1-ps1; Myo15b; Fgg; Ambra1;
Gm15446; Cyp2d34; Gm11644; Gpc3; Cotl1; Zc3h7b; Ap3d1; Gm14393; Prom2;
5730507C01Rik; Fbp2; Zfp934; Gm11009; Ulk1; Gm3531; Tmed4; Ntn4; Arl6ip5; Pttg1ip; Gns;
Gm13904; Pla2g15; Sult5a1; Pvrl1; Rnpc3; Gaa; Ndn; Ubald1; AU041133; Speer4cos; Cyp2d41-
ps; Tmem47; Gm1976; Clcnkb; Top3b; Zfp36l1-ps; Rnps1; Zfp935; Mndal; Pigt; Mapk13;
Fam92a; Trmt112; Scap; Gm21847; Anxa11; Adgrg1; Mapk3; Slc4a1; 4930522L14Rik; Ptprf;
Cdh1; Psap; 4930469K13Rik; Dpf2; N4bp1; AI317395; Clu; Carm1; Pisd-ps3; Man2b2; Sap130;
C2cd2l; Zfp932; Asb15; Aqp6; G6pdx; Abca3; Cd9-ps; Bmp6; Nprl2; Gm11675; Ctsa; Aff1;
Gm7935; St14; Rsrc2; Ipo4; Rogdi; Ubc; Trf; Cyp2d10; Gm5454; Btd; Wwp2; Pdk2; Cdhr5;
Psip1; Scgb1b19; Ezh2; Derl3; Syt17; Deptor; Cblc; Ankrd13a; Bcr; Twsg1; Tmem41a; Pisd;
Eef2; Kansl3; Abcc4; ND3; Gm2260; Hpcal1; Gm15487; Pax8; Got2; Tmbim6; Gm14408; Etv1;
Pnpla2; Gm10130; Gm10198; Cables1; Gm15784; Gm16356; Cyp2d11; Mrpl4; Rad9a; Eef1d;
Pot1a; Gm20939; Tsc2; Mif4gd; Bahd1; Nol6; Slco4a1; Rnf167; Gm2274; Sgms2; mt-Te;
Garem; Smarca4; Tom1l2; Git1; Crat; Gm15546; Pisd-ps1; Gm14239; Gt(ROSA)26Sor; C4b;
Cyp2d22; Gm14270; Gm4024; Ajuba; Dhrs3; Gria3; Fech; Folr1; Pld3; Gnpda1; Ddost; Mtor;
Gm5612; Sowahc; Gatsl3; Ncam1; Gm11838; Gramd1a; Ago2; Plekhb2; Dalrd3; Pllp; Inpp5a;
Slc2a12; Retsat; Shank2; Ttll1; Ptch1; Pax2; Grina; Osbp2; Slc7a8; Prdx2; Cyfip2; Gm6517;
Gm14400; Tufm; Stk38l; Chml; Plcg2; Stk11; Akt2; Msantd2; Tmem63b; Gm16379; Gm14401;
Cyp4fl3; Bcar3; Dhcr24; Trak2; Tmem184a; Prpf8; Eif4h; Fam234a; Miox; H2-Eb1; Trim8;
Polr2a; Ccbl1; Ube2v1; Sesn2; Pipox; Fbxo21; Trappc9; Mark2; Tm6sf2; Zfp51; Cdkn1a;
Gm23678; Sik3; Tex2; Gm7665; Gm22061; Gm5830; Asb9; D7Ertd443e; Gm6158; Gm5424;
Ube2v1; Camsap3; Wnk4; Galnt18; 0610040F04Rik; 3300002I08Rik; Chp1; Errfi1; Nrn1l;
Zfp760; Sap30bp; Suco; 5330417C22Rik; Wasf2; Gm15590; Ass1; Gpi1; Dnajc4; Cyp4a12a;
Snx3; Gas6; 5830428H23Rik; Hipk2; Cyp4b1-ps2; Vps28; Pex16; Taf1d; Arnt2; Gm22751;
Rab25; Gm27509; Trpm6; Gm14436; Tle4; Trim28; Etv5; Cyp2e1; Gm5871; Gm13650;
Arhgef11; Cyb5r3; Aldh2; Chrna4; Szt2; Sf3b2; Cwf1912; Gm13586; Dock8; Gulp1;
2610044O15Rik8; Lad1; Ift80; Gm12339; Slc29a3; Foxj2; Ttc14; Slc25a23; Lman2; Apoa2;
Zfp414; Wwc1; Adgre5; Cyp2a4; Gm10715; Scarna10; Got1; Inppl1; Mirlet7d; Fam149a; Bc1;
Gm20056; Man2b1; Gm11168; Spc25; G6pc3; Sgpp2; Hdac4; Rdh10; Furin; Tfcp2l1; Slc22a8;
Sppl2b; Gm5431; Slc25a28; Usp16; Ppif; Celsr2; Gm12727; Zfp101; Prrc2b; Vps50; Pyroxd2;
Pelp1; Gm16354; Gm5417; Myo7b; Mybbp1a; Gtpbp4-ps3; Akr7a5; Lpp; Ttll12; Gm26853;
Ubb; Edem2; Pcm1; Gm10720; Inpp4a; Comt; Fam160a1; Gsta4; Gm24168; Spint2; Gm6361;
mt-Tn; Dvl3; Arfgef3; Akr1c19; Shroom4; H13; Gm14399; Gm1821; Cpeb1; Gm8842; Zfp433;
9-Sep; Tecr; Proc; Pm20d1; Fam13a; Pcnxl3; Fbxw5; Gm16340; Tcea1; Ftl1; Proser1; Gm26239;
Uroc1; Yes1; Dleu2; Gm5228; Zfp948; Mir692-2; Vps11; Fam160b1; Gm22753; Gm12614;
Gm12624; Sqstm1; Gm2444; Gm12929; Prkd1; Smarca5; Tpp1; Gm21897; Gab1; Mkl2; Ndst2;
Gm17404; Zfp709; Txnip; Gm8910; Fam171a1; Gm13436; Atg3; Sp100; Unc93b1; Ksr2; Ten2;
Cyp2d13; Gm24714; Sh3bp1; Gm13139; Pkm; Arglu1; Gimap6; Gm11249; Slc13a3;
5430403G16Rik; Tlr1; Gm22444; Atp11b; Zfp433; Phf12; LOC100503279; Vps25; Sh3rf1;
Oaz1-ps; Slc25a39; Atp13a1; Ogdh; 1700120K04Rik; Gm14405; Slc25a3; Ndufa8; Plxna1;
Cdk4; Rfx1; Tpcn1; Kazn; Ppp2r1a; Slc7a7; Snx32; Fam120a; Gm8841; Dcn; Slc22a30; Znfx1;
Cpn1; Slc35g1; C1qb; Gm11008; Reps1; Ergic1; Nt5c2; Gm14420; Hltf; Gm14327; Llgl2; Tldc2;
Mfsd9; Gm12328; Cdh3; Eif2s1; Tmem37; Gm16581; BC021785; Gm12616; Pisd-ps2; Trmt112-
ps2; Gm6395; Ctsb; Zfp932; Dhrs4; Gm12704; Peg3; Gm14431; Gm14431; Gfra1; Pml;
Gm14000; Cpsf1; Scd2; Gm6743; Nup214; n-R5s41; Tex264; Mapk15; Agpat9; Gm25736;
Gm26779; Larp1b; Gm15012; Oxall; Zfp931; Zfp638; Apom; Timm17b; Slc16a2; Mta3;
Snora21; Aldh1a7; Gm10770; Gm5866; Ube4b; Slc7a4; Mif; 2010007H06Rik; Gm13204;
Cyp4a12b; Ampd3; Usp19; Slc36a1; Hoxb5os; Prpsap1; Mif-ps3; Mgrn1; Cyp27a1; Gm23407;
Foxi1; Gpaa1; Gm14308; Il4ra; Gm5761; Kmt2a; Anxa6; Crim1; Gm8700; Slc44a4; Gm10800;
Gas5; Gm6576; Gm25326; Laptm4b; Dmrt2; Igf1r; Sox6; Macrod2; Rock1; Adi1; Rpl29-ps2;
Gm8648; Mir30b; Bglap3; Sclt1; Tmem252; Rfng; Zfp72; Gm14409; Cyp2u1; Zfp125; Trrap;
Gm26465; Gm25021; Gm24864; Gm24424; Gm24389; Aar2; Eef2kmt; Gm13139; Acox1;
Otud6b; Dsel; Pdlim3; Ndrg1; Gm13248; Zc3hav1; 2310061I04Rik; Gsk3a; Sgta; Slc25a10;
Gm14421; LOC100861620; Gm14322; Cyp2d9; Lmtk2; Lmf1; Gm11449; Gm13235; Gm14431;
Zfp781; Slc6a19; Kng2; Gm8615; Smarcc2; Vps33a; Gm1840; Hmga1-rs1; Gm3414; Rps2-ps10;
Prom1; Vdr; Gm5637; LOC100862386; Cpt2; Gm14188; Gm14036; Bc1; Etnk1; Tbrg4;
Cep170b; Gm5786; 4833439L19Rik; Wsb2; Gm12723; Cd9; Met; Thoc2; Susd2; Ctsz;
Gm13294; Ska2; Cdk10; Erbb2; Serpinh1; Pum1; Sall3; Tpra1; Gm22137; Creb312; Dtnbp1;
Fam65a; Med21; Spred2; Gm13152; Zc3hav1l; Zfp945; Arhgdia; Clmn; Anpep; Ssb; Gtf3c2;
Cnot8; Guca2a; Nav2; Ttc39c; Sympk; Gm3411; Klhl28; Ptpn3; Prkca; Rnf31; Abr; Lypla2; Pxn;
Ap2a2; Acaa2; Gm23140; Cog1; Gm16357; Shmt2; Dclre1b; Bglap2; Ubl7; Gm21738;
Gabarapl1; Gm14537; R3hdm4; Gm5921; Gm23455; Gm10719; Gm10717; Slc2al; Mir692-3;
Gm22392; Prr15; Wbp2; Leng1; Gm6344; Uqcrc1; Smc6; Gm21411; Gm25401; Trappc10;
Cdcp1; Papss2; Maged1; Atp6v0c; Gm5070; Trpv4; BC034090; Fam132a; Gm13127; Hhatl;
Nelfb; Gm17150; Trpt1; Celsr1; Agap1; Pdxk; Gm5513; Cops7a; Gm10563; Carkd; Bicc1;
Rnf208; Gm11533; Zfp930; Gm12615; Elk3; Cited2; Gm11336; Nt5dc3; Slc25a15; Fam83g;
Plekhm1; Sema4b; Nfix; Gm23814; Gm14107; App; Dis312; Etohi1; Gm22969; H2-Ab1; Tuba1c;
Pacs2; BC035947; Zfpl1; Pgam1; Tmem259; Gm25205; Rpl14-ps1; Plin3; Rab4b; Mir6386;
Atxn2; Fam195b; Trp53; Slc22a28; Gm26331; Gm9140; Itpr2; Ctbp1; Dock9; Polrmt; Gm11902;
Dnajb12; Rnf185; Gstp1; Galns; Gm14288; Gtf2h2; Pcif1; Trp53-ps; Slc35a4; Rps2; Dpagt1;
Gm20746; Gstp2; Slc16a13; Mboat1; Gm22968; Gm4968; Prex2; Kng1; Aldh3a2; Acad10;
Gm11843; Slc9a1; Gm27828; Pard6b; AI987944; Sbf1; Rmi1; Magi2; Gm14387; Gm15968;
Gm23264; Cldn6; Eif4ebp2; Ddx39b; Ngef; Zfp131; LOC100862331; Clk1; Tmed10; Plagl2;
Gm23369; Reep4; Gm16177; Gvin1; Gm17535; 2310007B03Rik; Ern1; Eif4b; Spns2; Psmd3;
Nprl3; Rbpms2; Gm8765; Gm3285; Plpp3; 2310022A10Rik; E2f4; Clstn1; Slc19a1; Gm25772;
Sipa1l3; Gm12619; Eno1b; Map2k2; Naga; C4a; Adgrf5; Mbtps1; St6galnac2; Rpl28-ps3;
Mpv1712; Tm9sf4; Slc27a1; Stat5b; Gm24098; Gm25381; Mir692-1; Cs; Rabggta; Ncor1;
Gm15962; Zfx; Eif2ak2; Tctn2; Fam73a; Zfyve26; Ncdn; Gm14411; D630045J12Rik; Gm17421;
Zfp52; Wrap53; Lhx1; Gm20775; Mirlet7a-1; Imp4; Rptor; Ppp1r37; Gnb2; Rnf123; Fopnl;
Epb41l1; Ifi203; Chic2; Hps4; Gm16010; Rpl17-ps9; Kansl11; Mrpl36; Btbd8; LOC100861682;
Hs6st1; Gm13241; Tnpo2; Gm14111; Acsf3; Uhrf1bp1; Gm15536; Gm25577; Plcb3; Phpt1;
Gm14403; Gm5561; H2-DMb1; Gm18733; Gm13242; Numal; Gm25992; Gm24267; Coro7;
Gm10088; Got2-ps1; Mir3471-2; Mir3471-2; Itgax; Gm4944; Athl1; Snhg6; Samd9l; Rps2-ps4;
Ppargc1a; Cstf1; Snap91; Atraid; Znhit2; Dapk1; Ints9; Gm25355; Gm23064; Gm14325;
Gm10420; Tmem50b; E230008O15Rik; Pgm2; Gm11687; ND6; Clk4; Eps8l2; Sgsm1;
Tmem86b; F830001A07Rik; Gm22474; Gm3470; Gm3993; Myof; Gm22132; Mmachc; Ncstn;
Fbxo30; Gm2004; Haus4; Arap2; Kdf1; Iws1; Cyp2a5; Tnfrsf1a; Gm10801; Calcoco2; Gm13022;
Sidt2; Gm14516; Gm5270; Dio1; Fam63a; Dap; Exoc5; Cobll1; Cep83; Gm3500; Lrp5;
Gm23815; Gm3756; Eno1; Cnnm1; Uba1; Farp1; Gm9774; Abhd5; Gm6485; Ubr4; Arsa; Ces2e;
Zfp523; Igf2r; Gm11034; Zcchc11; Nav1; Wdr83os; Gm24715; Mir26a-1; Fuca1; Gm12276;
Pnkd; Zfp326; Mob3b; Gm25337; Slc6a6; Gm25872; Tm7sf3; Gm29376; Gm22821; Gm25484;
Gm24641; Gm17631; Gm6712; Jmjd8; Dusp7; Capn7; Tmem174; Eri3; Cherp; Mfsd4; Gm4735;
Xpo6; Plekhg3; Gm11868; Zhx2; Suz12; Dek; Lpcat3; Scube3; Eif3d; Gm14288; Pi4kb;
Gm10203; Gm6139; Wdyhv1; Vapa; Igkv8-30; Zdhhc4; Gm8580; Aplnr; Pde7b; Gm21833;
Ncln; Zfp62; Zcchc14; Capzb; Nicn1; Calb1; Rdh11; Gm7189; Mir1981; Orc2; Os9; Gm10222;
Rpl18; Pkp4; Wdr81; Gm1965; Rbm39; Tmem8; Umod; Ech1; Mdh2; Prdx2-rs3; Mta1; Pcolce;
Otub1; Acp6; Dym; Hnrnpdl; Smcr8; Lama3; Rmnd5b; Rtfdc1; Polr3a; Nmnat3; Lztfl1; Gm5978;
Gm2808; Gm12444; Hist1h1c; 4930453N24Rik; Pias4; Gm6311; Gm19425; Foxn3; Gm16061;
Gm14712; Atg2b; Gm14431; Map1b; Fam134a; LOC100861804; Cxxc1; Lysmd3; Ube3b;
Polr1b; Pxk; Ahsa2; Ezh1; Gm10254; Anxa4; Psmc6; Gm22463; Gm25972; Gm22547;
Gm23976; Gm26386; Gm25458; Gm23292; Gm22414; Gm23646; Gm22825; Gm26241;
Gm24973; Gm22913; Gm25084; Gm23282; Gm24302; Gm24643; Gm26235; Hgs; Pfkp;
D10Jhu81e; Rps2-ps6; Usp20; Gm14416; Clcn6; Gm12451; Dda1; Idh2; Gm22129; Zdhhc8;
Gm12881; Lyrm7; Alpl; Rpl14; Ccdc173; Reps2; Bpnt1; Ercc6l2; Iqsec1; Noc4l; Gm6445;
Tanc1; Ap5s1; Gm12480; Rpl29; Rgs5; Snhg5; Vrk3; Gm19722; Acnat2; Ano6; Uimc1;
Gm14480; Gm23062; LOC100505224; Dync1h1; Gm14306; Gm3852; Ifi30; Cyb561a3; Mthfd1;
Zswim1; Pigr; Rpl39-ps; Gm13226; Gm7353; mt-Tp; Cisd1; Klhl3; Gm12502; Rai2; Mdk-ps1;
Tmem175; Smad1; LOC100861810; Gm25132; Gm6665; Gm10721; Cxx1c; 4930563D23Rik;
Herc6; Ccndbp1; Tcea1-ps1; Degs2; Gm10094; Smim6; Ggt1; Gm13921; Clptm11; Fat1;
Gm13841; Gm17705; D630045M09Rik; Cat; Rbm26; Zfp809; Stambp; Rhcg; Rps6ka1; 7-Sep;
mt-Tq; 2310001H17Rik; Mysm1; Fah; Ddx47; Arl8a; Ddx24; Nr3c2; Myo9a; Pigo; Rpl18-ps2;
Ssbp1; Gm17131; Crtc1; Phf2; Gm17604; Dnaja1; Ltc4s; Zfp654; Fzd4; F2rl1; Hook3; Hipk1;
Ndufa10; n-R5s180; LOC105244763; Gm6682; Sipa1l1; Lrrc8d; Chchd6; Cmip; Phf20l1; Copa;
Smoc1; Gm25214; Pigq; Gm10586; Pgd; Gm14405; Nnt; Ago1; Rpl26-ps4; Rps2-ps5; Gm22507;
Zfp626; LOC100861882; Gm25080; Mvb12b; Prkcz; Gm10913; Pdgfb; Ncoa1; Gm4917; Dock4;
Rock2; Gm12091; Il10rb; Ptbp1; Gm12193; Synj2bp; Cul5; Slc25a35; Gys1; Bsg; Gm23192;
Gm19313; Rbm12b2; Bmp8a; Nop58; 1110059E24Rik; Mrpl2; Itga8; Ktn1; Gm23095; Parg;
Gm12622; Arfgef2; Pgam1-ps2; Fam98a; Mavs; Mfsd7a; Prr13; Gga2; Gm12627; Ncoa2;
Gm23144; Yif1a; Gm11852; Gm20290; Rsl24d1; Wdfy3; Gm24888; Maea; Rpl29-ps5; Zfp322a;
Gata3; Rac1; Gm13740; Gm18859; Zfp960; Ddx49; Pmm2; Vbp1; Ptpn23; Arsb; Rnd3; Snrnp40;
Tstd1; Zfp329; Gm19541; Pigl; Prkag2; Gm24539; Letmd1; Gm5619; Gm13094; Pmf1; Oaz2;
Gm17035; Eif3g; Gm24614; Gm26297; Esf1; Pfn1; Lrp10; Csnk1d; Gm2573; Gm16124; Abhd4;
Gm15429; Aldh1l1; Gbf1; Luc713; Tmem136; Habp2; Gm26501; Gm7367; Dpy1913; Ppp1ca;
Gm14391; Igsf11; Fads1; Bc1; Gm3934; Gm11966; Gm10193; Slc38a10; Smc5; Gm21750;
Aplp2; Slc16a4; Sparc; Gm13316; Ubqln2; Sec31a; Gm27541; Gm20165; Gm9761; Cdc14b;
Tmem143; Dgkg; Gm22306; Cabin1; Gm13669; Gm14870; Gm16477; Gm10718; Ergic2;
Csnk2b; Tmem171; Gm5046; Gm14418; Gm23648; Rps16; Gm3250; Gm21962; Adck5; Scly;
Mir5111; Gm13213; Ints1; Dcaf5; Sim2; Gm23137; Tifab; Rif1; Hist1h2al; Muc20; Nabp2; Arsg;
Scrt2; Zfp395; Gm15883; Tfec; Sufu; Papss1; Ppp1r15a; Gm23690; Gm14288; Scamp2; Ofd1;
Ttyh2; Nsa2-ps2; Gm24462; Gm10787; Tcf25; Gm12439; Alox12; Atp6ap1; Vkorc1; Wdr91;
Copb1; Gm23934; Gm24182; Gm25829; Gm22360; Gm25583; Gm26496; Gm22295; Gm25869;
Gm22682; Gm25127; Gm24458; Gm22798; Gm24260; Gm24978; Gm24967; Gm22092;
Gm25737; Gm26058; Gm25405; Gm24414; Gm25790; Gm22822; Gm24693; Gm26427;
Gm24205; Gm25064; Gm22288; Gm22580; Gm26317; Gm25256; Gm24486; Gm24019;
Gm26243; Gm25709; Gm24803; Gm24859; Gm22246; Gm22170; Gm23296; Gm24873;
Gm22823; Gm24540; Gm24753; Gm23217; Gm26295; Gm25547; Gm24722; Rnu6; Gm23512;
Glmp; Hax1; Cep70; Gm24863; Mir7027; Gm27825; Gm28023; Naaa; Adrbk1; Gm11405; Pcx;
Gm906; Klhdc3; 4931402G19Rik; Whsc1; Nfx1; Nr0b2; Top2b; Bcas3; Gm6054; Zfp3611;
Gm20899; Tubd1; Anapc10; Fam43a; Gm20267; Bag6; Vps13a; Gm25395; Gm22618; Txlng;
Ndor1; 1700025G04Rik; Epha2; Dhrs1; Fbxl18; Gm11860; Stat3; Jchain; Gm2026; Gm12168;
Kat7; Smim10l1; Preb; Ndufaf4; Fzd2; Gm26128; Cpb2; Npc1; Mcoln1; Gm26716; Baiap2l1;
Gm5762; Dab2ip; Gm15800; Sema4d; Dusp6; Gm13157; Slc1a1; Ifi44; Gm14412; Gm23189;
Gm25708; Gm25959; Gm24332; Gm23802; Gm22903; Gm25571; Gm23049; Gm24370;
Gm24159; Gm25299; Gm23798; Gm25070; Gm22475; Gm23400; Galt; Ppp1cb; Nckap1l; Tek;
Zfp942; Gm12397; Wdsub1; Caap1; 1700028J19Rik; Flt4; Fibp; Sssca1; Gm12421; Iqgap1;
C130026I21Rik; Dusp1; 2210418O10Rik; Gm22550; Tubb2a-ps2; Gm5239; Usp30; Gm20702;
Fbxo9; Mir3471-1; Gm24095; Gm22633; Cldn2; Gm22740; Gm13818; Arhgap18; Camsap1;
Fam168b; Gm6407; BC022687; Slc25a40; Gm12484; Gm15773; Lypla1; Gm12613; Rplp0-ps1;
Lypd6; Slx1b; Nlrp6; Eri1; Mir5100; Fam189a2; Gm6505; H2afy; Smek2; Hoxd11; Ddc;
Gm10169; Gm12341; Pex13; Tbc1d10a; Gm25297; Zfp715; Amot; Galnt9; Cd3e; Cnot6; Gps2;
Gm22370; Nasp; Clptm1; Nelfa; Gm26870; Tamm41; Guf1; 1700060C20Rik; Nutf2-ps2;
Gm14034; Gm8738; Nedd4l; Papd7; Slc12a7; Orc4; Col6a3; Arhgef16; Large; Gm10105;
Gm14312; Gm26231; Gm22234; Gm15878; LOC100862216; Slfn3; Hsp90ab1; Pcyox1; Slc3a2;
Gm22504; Dip2c; Ubiad1; Gm22749; Ubac2; Mir494; Lama1; Alg3; Ppargc1b; Bglap;
BC003331; Dock7; Pcbp1; Nhsl1; Gm11847; Tmem72; Rpl14-ps1; 5730405O15Rik; Ubap2;
Poldip3; Mrpl38; Gm22616; St3gal3; Poldip2; Rab11b; Ppp2r5c; Utp14b; Bco1; Wfdc15b;
Hist1h2an; Farp2; Gm17555; Rpl7a-ps10; Pcdh1; Dennd1a; Gm11539; Polr2e; Ms4a6c; Pom121;
Vstm2a; Ercc4; AU040320; Igha; Gm904; Nrg1; Kmo; Mir99a; Mir5098; Nisch; Cyp24a1;
B4galnt3; Piezo1; mt-Tr; Ralbp1; St5; Vezf1; Zranb1; Skida1; Tubg1; Mtfr1l; Ush1g; Cbfa2t2-
ps1; Gm11512; Ndufs2; Cdh16; Osgepl1; Mbtd1; Dctn3; Smarce1-ps1; Rqcd1; Sarnp; Rassf4;
Alkbh7; Srxn1; Chdh; Gm15662; Rpl10-ps1; Gmppa; Cog7; Gm5455; Akirin2; Gatad2a; Hnf1a;
Hnrnpul1; Zfp963; Krit1; Tgm2; Gm19474; Gnai2; Xpr1; Oaz1; Gm9166; Dzip3; Gm14326;
Slc47a1; Pigm; Gga1; Matr3; Gm24949; Wdr5; Gm13051; Prr14; Ace2; Dmxl1; Ergic3; Sat1;
Pcdhgb1; Heatr5b; Dcun1d1; Gm13416; Fam193a; Gm5093; Alg1; Gm7286; Gcc2; Atp6v0a1; n-
R5s56; Foxk1; Pef1; Gm3695; Rps2-ps9; Gm22623; Cwh43; Gm13882; Pank3; Ift43; Ndrg3;
Gast; Keg1; Rpl10-ps6; Gm3272; Tmem173; Tut1; Gm13864; Mttp; Cyp4f17; Ppp2r2c; Kctd12b;
Gm11172; Cd200; Gm20559; Wrb; Mrpl34; Tmx3; Slc39a9; Nudcd3; Chpt1; Ranbp3; Gm11605;
Gm12892; Hpn; Gm2308; Gm24565; Gm23966; Slc7a9; Zfand5; Lrrc59; Lrrk1; Gm7137;
Rab11b-ps2; Gm6969; Sdhc; Gm12864; Chmp3; Tmem181b-ps; Gba; Mospd3; Gm14419;
Rnu3b4; Pi4ka; Gm8258; Rragd; Stat5a; Midn; Gm23269; Gm22307; Aldh7a1; Hmgn5; Pkn2;
Mrpl14; Slirp; Gm5430; Tinf2; 4833418N02Rik; AI464131; Gm12625; Cdc42bpb; Rap1a;
Cyp2d40; Trim12c; Alkbh5; Ercc2; Eci2; Myo5a; Rnu3b1; Rnu3b3; Rnu3b2; Mir3091;
9530026F06Rik; Gm15467; Pcnp; Ndufb2; Ppp2r5a; Nipal2; Whrn; Gm10524; Mir30d;
Tmem251; Aldoa; Actb; Gm26951; Zfp592; Actn4; Ski; Mettl21a; Ssbp3; Tmco4; Isca2; Eif3j2;
Tnrc6c; Usp10; Pam; Rpl7a-ps3; Rpl7a-ps5; Gm6433; Maf1; Gm8995; Rnpepl1; Gng7; Cd209c;
Alb; Gm15443; Rbbp9; Gm12422; Mfsd11; Gm14723; Slc39a1-ps; Zfp97; Gm23162; Gm20645;
Mir1938; Eif6; Sdr39u1; Nit1; Hibch; Copg1; Mir8115; Bod1l; Gm20252; Gm12922; Caskin2;
Phtf1; Gm12403; Hist1h2af; Gm23579; Gm24646; Rexo1; Slc25a17; Wdr13; Naa15; Sfxn1;
Gm13394; Gm23057; Cirh1a; Abhd3; Psen1; Gm14278; Calcr; Rps13-ps1; Arf1; Taok3;
Hist1h2aj; Gbp4; Them4; Epg5; Map3k15; Gm17184; Pnpla7; Gpx1; Ids; Adra1b; Ppp6r2;
Gm10021; Polb; Gm12669; Vps37a; Rfxank; Gm12033; Gm22531; Ctns; Cxadr; Mitd1;
Gm14180; Gm4581; LOC100861762; 5730409E04Rik; Rpl8; Tmem176a; Pacsin2; Nup133;
Stau1; Tmem87a; Vac14; Map3k5; Cope; Atp6v1b2; Plekhm2; Kras; Tmem180; L1cam;
Gm15355; Aldoart1; Gm11684; Ppp1r1b; Gm4895; Gm11989; Tmem106a; Hmgcs2; Peg13;
Tln2; n-R5s213; Ifih1; Cbl; Gm22906; Ndufv1; Mir7005; Sfxn3; Nedd9; Sema4a; Gm12050;
Gm7027; Zfp386; LOC100861904; Gm24695; Chchd10; Gbp7; Agrn; Gm2445; Hnf4a; Egln3;
Gm8623; Tle3; Gm12162; Slc22a23; Morc3; Notch2; Gm5559; Med16; Dennd4b; Arid1b;
Gm25986; Tmem248; Smim14; Lgmn; Gm25435; Rasa1; Sox9; Cuta; Cnksr3; 2610305D13Rik;
Gm16470; Gm26340; Wiz; Perp; Gm13123; Suclg2; Gm5459; Herc3; Taz; Tctn1; Babam1;
Gm15191; Pcf11; Med15; Gm15846; Gpam; Gm8318; Pik3r2; Mir22; Tox; Gm23244; Fxyd1;
Zfp958; Gm26463; Gm24975; Gm25100; Gm28019; Gm24307; Qars; Apob; Armc10; Cda; Bvht;
Gm12621; Isyna1; Tmem181a; Btbd2; Gm5621; Rbbp8; Gpbp1; Cldn8; Elmod2; Mnda; Rnf114;
Gm13588; Taf8; Nsfl1c; Ing5; Clcn7; Ctsc; Cox5b; Pcyox1l; Gm12999; Wwc2; 9330159M07Rik;
Rab18; Faah; Nr1h2; Cacnb2; Mycbp; Gm23924; Gm23744; 2900026A02Rik; Gm11448;
Rbms3; Mroh1; Gm25563; Gm22852; Tbc1d9; Gm22450; Akr1c21; Mdk; Zfp729b; Kcnj10;
Gapdh; Marcks; Gm12040; Igfbp7; Ift74; Gm25412; Dusp18; 9530068E07Rik; Gm7138;
Gm10451; Ehbp1; Pkd1; Phb2; Dqx1; Gm7808; Fig4; Gm12821; Amt; Ifrd1; Mir6911; Ethe1;
Abca2; LOC100862018; Xpo5; Gm14856; Gm24984; n-R5s165; Ncoa3; Gm6498; Tmem176b;
Btrc; Tmem192; Htra2; Mir320; Gm10051; Kcnc2; Cdk6; Prkrir; Gm25799; Igkv4-56; Porcn;
Gm22505; Gm22363; Ckmt1; Cln8; Gstt3; Gm4875; C1d; Gm23610; 4921524J17Rik; Slc27a2;
Ube2d4; Pidd1; Serinc3; Usp14; Camta2; Arhgef7; Calm2; Prmt2; Scnn1a; Dhx37; Rps11-ps1;
Ralgps1; Gm15638; 2810403A07Rik; LOC100861863; Rnf25; Ino80d; Tmem63a; Gm15173;
Gm18025; Gm13837; Rcan2; Akap1; Rps11-ps3; Gm6712; Gm12565; Chst2; Sgsm2; Gm6564;
Bnc2; Tmem127; Gm22338; Cox4i1; Ercc1; Gm8806; Gm14277; Prdm16; Sfl; BC002059;
Smarcd1; Gm22114; Prosc; Appl1; Mir6957; 1700122H20Rik; Zfp280d; Gm23520; Gm16433;
Bud31; Tjp2; Pdss1; Magi1; Gm14308; Sars2; Gm24271; Atp5sl; Gm24928; Pip5k1a; Gm17146;
Gm26545; Fas; Gm11488; Pccb; Rad51d; Ddah1; Phyhip; Taf3; Trabd2b; Thrb; Mansc1;
Guca2b; Cspp1; Alkbh3; Gm2574; Gm23329; Ppwd1; Gm21954; Cpsf4l; Gtf3c1; Lama5;
Higd2a; Hkdc1; Ruvbl2; Glis3; Elf1; Gm23553; Abhd17c; Gm7973; Plpp2; Gm24812; Slc8b1;
Prkag1; Plvap; Deaf1; Gm5884; Rpl28; Ap2m1; Gm14391; Iscu; Stk32b; Cby1; Lcp1; Gm6493;
Tbxas1; Tmf1; Gpc4; Gm3552; Med25; Rprd1b; Abce1; Cond3-ps; Hadh; Gm15378; Gm14311;
Ccnd1; Traj14; Gm14308; Emx1; Kxd1; Htra1; Nemf; Shmt1; Sf3b4; Araf; Mpdu1; Wwp1;
Gm11914; Gm8225; Cul4b; Idnk; 2010111I01Rik; Col4a2; Msra; Uba52; Zc2hc1c; Smarcd2;
Slc22a1; Snap23; Ankrd39; Blvrb; Tns1; Plbd2; Cdv3; Ppfibp2; Prpf3; Ppt1; Baiap2; Itpkb;
Neurl2; Gm26944; Ube2v2; Zf12; Echdc3; Zfp961; Baz2a; Gemin2; Hif1a; Sec61g; Orc6;
Gm23507; Gm12009; Ugt2b37; Cyp2d37-ps; Gm6808; Tmem168; Hist1h2ag; Ptgr1; Prr5;
2010111I01Rik; Gm14295; Ascc2; Cebpa; Gm14176; Stard5; Mki67; Gm6283; Thoc3; Fam188a;
Gm7860; Gm20483; Myrf; Rps16-ps2; Sil1; Coro1c; Spint1; Nbeal2; Nomo1; Bbs1; Pip5k1c;
Gm22951; Fmo4; Kap; Gm9625; Gm15578; Gm24357; Ankrd40; Gm23766; Pdxk-ps; Aqp1;
Chac2; Spg7; Pnmal2; Atf7ip; Fbxl6; Aup1; Treh; Wsb1; Adam11; Gm11475; Kif3b; Rhot2;
Gm12115; Ddx10; Kcnip2; Ighm; Prima1; Gm5507; Gm10481; Mir3100; Cmpk1; C2cd3;
Snapc3; Acad12; Hyal2; Slc6a9; Eps15; Rcor3; Gm4518; Gm26138; Cenpj; Hbegf; Gm16755;
N4bp2l2; Klhl9; Luc7l2; Gm24689; Prpf39; LOC100861805; Cptp; Gm15981; Rps11-ps2;
Ino80c; n-R5s9; Gmds; n-R5s25; Smek1; Crebbp; Dpy19l1; Styx; Rps13; Coq5; Cars2;
Gm11270; Cept1; Gm22060; Mir378c; Casc4; BC005512; Fh1; Emd; Igkv1-117; Gm25551;
2610042L04Rik; Gm14612; Apoa1bp; Phf11b; Acsl1; Tmem220; Gm3809; Gm13822; Abat;
Pomt2; Gm6286; Gm3238; Mal; Mreg; Klhdc9; Ddx56; Lrrcc1; Gm16436; Zfp185; Gm22730;
Gm26153; Snx17; Tmem181c-ps; Ptcd3; Ildr2; Csrp1; Arhgef3; Gm15516; Dbt; Pcbp2; Gm128;
Smtn; Gak; Aven; Mettl7a1; Ciz1; Gm17149; Def8; Gm14450; Gm14308; Glrx3; Ppm1h;
Gm14150; Gm17576; Col14a1; Fadd; Hpd; Gm14308; Frrs1; Osgep; Rwdd4a; Bhmt-ps1;
Gm20342; Kiss1r; Nol8; Nes; Gm25203; Rtn1; Smndc1; Gm12419; Kat2a; Rpl39; Cox6a2;
Nr1d1; Atp2b4; Tpcn2; Fam25c; Rnf183; Gm22628; Birc2; Mrpl20; Traf7; Smpd4; Sh3tc1;
Gm25450; Mink1; Calcoco1; Fam151a; Fbxl8; Tbl1x; Hsd3b7; Ubqln1; Zdhhc9; G6pd2;
Gm24573; Dcxr; Cnot1; Gm12245; Kat8; Sephs1; Gm14733; Tab2; Kcmf1; Zufsp; Meox1;
Cpeb2; Csnk1g2; Gpbp1l1; Dnm2; Car4; Gm12443; Sigirr; Mfsd1; Gm22596; Rbm12; Med24;
Reep6; Gm3788; B630005N14Rik; Rabgef1; Npy; Tatdn1; Ndufs8; Foxd2os; Zfp429; Wdr45;
Coq2; Tspan31; Mir6546; Tox3; Rhoc; Csnk1g3; Gnb1; Gm1947; Terf2; Dab2; Nsmce2;
Smarcal1; Gm3233; Ift46; Gm24311; Gm24295; Gm26430; Ap2b1; Gm13319; Gm7336; Mrpl1;
Mir5105; Mark3; Rasa2; Rp2; Prpf4; Gm11599; Eaf1; Mrpl35; Alad; Egr1; 2210404O09Rik;
Gm23103; Gm21399; Mir483; R3hdm2; Gm17199; Gm24239; Igkv1-110; Abca8a; Gm5834;
Ntmt1; Gm12366; Gm6999; Eif3i; Crtc2; Trappc12; Zmynd10; Gm12755; Kcnq1; Zfp764;
2510039O18Rik; Bphl; Gm13381; 2510002D24Rik; Eif4a3; Map3k3; Mplkip; Snord8; Trib1;
Arnt; Ifi204; Vwa9; Gm23649; Gm12933; Vnn1; Gm12991; Slc9a8; Mtmr3; Lrp11; Myo7a;
Gm2606; Sord; Vill; Gm8731; Gm22249; Golim4; Amot; Gm19631; Mzt2; Gm12063; Esrrg;
Rnu3b2; Rnu3b3; Rnu3b1; Mkl1; Man1b1; Mir6995; Zfp617; Gtf2h3; Thumpd3; Pogz; Ist1;
Gm26197; Plk3; Sin3b; Gm11583; Slc22a22; Sec61a1; B4galnt1; A730017L22Rik; Dlec1;
Gm25448; Gm24713; Ptprk; Gm24710; Gm24135; Pdcl; Gm13434; Gm8832; Anks1; Gm20492;
Ogn; Gm23992; Fam208a; Zfp281; Gm12122; Mrpl28; BC024416; Camk2g; Nup93; Gm11349;
1810019D21Rik; Gm24336; Gm13145; Polr2k-ps; Dcaf7; P3h2; Gm20472; Blnk; Spns3; Hba-
ps4; Gm25453; Atp5a1; Mir7066; Mir453; Gstm1; Tbc1d8b; Rnu6-ps2; Adat2; Gm2976;
1700119H24Rik; Arhgef10l; Txnl4a; Rab22a; Gcn1l1; Gvin1; Zfp142; Gm15621; Prdx6;
Gm10284; Vpreb2; Tnfaip1; Lilr4b; Atg4b; Rrp8; Itm2b; Gtf2ird1; Snord72; Nadk; H2afv;
Tmem116; Tom1; Atg4a; Tpm3-rs7; Cox18; Acot1; Fads2; Mtmr4; Trak1; Epn1; Gm11203;
Clec2g; Rce1; Rap1gap; Gm10356; Gm27317; 0610009O20Rik; Trav13-4-dv7; Gm10359;
Rnf44; Ighv1-5; 4930444E06Rik; Col3a1; D630029K05Rik; Gm16305; Tbc1d22b; Mcm5;
Gm14548; Tekt2; Gm17546; Champ1; Irak2; Gm25675; Spopl; Gm9034; Gm5225; Dusp15;
Idh3g; Tmem61; Mir1950; Gm11470; Rpl4; Gm22950; Gm4691; Ttc7b; Anxa5; Cisd3;
LOC215866; Rexo4; Hsdl1; Copz1; Prelid1; Poc1b; Nfyc; Vegfb; Med9; Tapbp; Impad1; Ap4e1;
Entpd4; 2310068J16Rik; Paip1; Agpat1; Gm6316; Dnase1; Ppy; Klf7; LOC102631757; Gm6565;
Ecsit; Gm4335; Gm14305; Nacc1; Fnbp11; Apela; Phf14; Rmdn2; Ogfrl1; Pgrmc1; Ypel3;
Zfp597; Gm14165; Mir340; Dpp9; Gm23308; AI838599; Gm3534; Gm17099; Adck1; Gm22743;
Pdzd2; Gm22009; Gm24011; Pamr1; Sorbs1; Gm13252; Gm11992; Mrps26; Slc35e1; Maged2;
Ipo13; Pnn; Ccdc90b; Gm13036; Nub1; Gm12671; Gm15163; Ldhd; Cachd1; Gm12537;
Mir449b; Gm12739; Arhgap39; Gsto1; 5730408K05Rik; Sigmar1; Plp2; Synrg; Gm6415; Rbl2;
Eefsec; Atp6v0d1; Slc26a1; Fcgr3; ND4; Tubb2b; Txn2; Mfsd10; Spin4; Epb41l3; Sp3;
Gm10775; Notch1; LOC100862298; Dera; Tmem102; Gm11808; Lcorl; Gm15343; Psme4;
LOC100861887; Dpp4; Cdk16; Tmprss2; Slc37a1; Zdhhc18; Gm26448; Comtd1; Gm7308;
Kif5b; Wfdc2; Adm; Gm4575; Thbs1; Aldoc; Dnajc16; Aig1; Rad23a; Smo; Gm5138; Mir335;
Slc4a2; Gm26125; Ap2s1; Slc35c1; Zzef1; Gm23092; Gm24242; Gm20004; Acot12; Gm10042;
Gm24288; Gstm2; Gm12783; Cyba; Ces1f; Ipmk; Hdac6; Sh3gl2; Nutf2; B230307C23Rik;
Gm19826; Polr2k; Capns1; Gm9013; Ap1b1; 2610016A17Rik; Gm10819; Tmem69; Gm10358;
Copz2; Esrp1; LOC100862140; Cbr1; Zfp229; Ppcs; Kpna1; Gm23948; Rps8-ps1; Gm7902; Etfb;
Tuba4a; Gm22756; Gm22003; Gm19427; 4930481B07Rik; Olfr1396; Hmgb1-rs17; Atp2a3;
Gm23154; Ubqln4; Mir7091; Gimap3; Ubxn8; Tm2d2; Magea2; Phyhipl; Dlg1; Pim3; Ighv1-34;
Homer1; Marveld1; Tm4sf1; Esco1; Gm13262; Gm25247; Tgfbrap1; Gm6901; Tollip; Slc25a46;
Bop1; Eci1; Kremen1; Sult1c2; Add3; Hsp90aa1; Rnf168; Adgra2; Slc25a1; Atp6v1b1; Iqce;
Asb8; Gm22671; Laptm5; Tbc1d9b; Fam20b; Plppr2; Trim63; Myd88; Gm24119; Mtf1; Zfp442;
Tmem150c; n-R5s195; Sgf29; Gm8330; Exosc10; Ift27; Gm22578; Malrd1; Ddx19a; Zdhhc12;
Gm16580; Mir6910; Psmb2; Slc41a1; Gm27626; Dad1; n-R5s155; 1700040L02Rik; Gm12896;
Fstl1; Gm10566; Ddx54; Gna12; Gm15786; Cops2; Adcy6; Pibf1; Slfn5; Rnf216; Gm15538;
Pigb; Stambpl1; Rb1cc1; Npr2; Rab38; Ptpn21; E130309D02Rik; Hnrnpu; Myo1c; Gm24741;
Uhrf2; Sumo3; Ube3a; Slc45a3; Gm17828; Sfpq; Slc24a3; Gm17538; Nbn; Oasl2; Gm14946;
LOC100862373; Fgfr4; Ept1; Usp1; Prok2; Thap6; Zbtb20; Avpr1a; Tmppe; Pou2f1; Upb1;
Ushbp1; Tspan7; Rnf225; B230325K18Rik; Letm1; LOC100862199; Slc38a4; Rps9; Fkbp7;
Pde4dip; Scarb2; 9430016H08Rik; Vwa5a; Gm5976; Zbed4; Ighv1-12; Gm19648; Rnf138;
Gm2423; Yipf3; Capn1; Gm3242; Mllt1; Zfp637; Casr; B4galt5; Pcsk5; Myl12a; Parl; Gm12112;
Tmem201; Cfap36; Gapdh-ps14; Nbeal1; Gm5555; 2410004B18Rik; Prpf40a; Hba-a2;
Gm14182; Gm4540; LOC100862112; Gm11807; Ubap2l; Fkbp3; Edaradd; Ccnyl1; Hmbs;
Abhd6; Ppil3; Slc35e3; Cmc2; Gm23888; Smad9; Usp36; Gm2310; Gm5426; Gm17428;
Tmem52b; Gm10777; Tbc1d19; Gm26112; Slc22a15; C1qtnf7; Anapc5; Neu1; Pdss2; Mir7087;
Zfp524; Mocos; Sirt5; Gm19545; Ly6f; Gm14760; S100g; Cog6; Cox7a21; Gm25467; Dnajc14;
Map1lc3b; Strada; Eif4a-ps4; Zfp825; Gm12582; Glb1l2; Gm23257; C330006A16Rik;
Gm24078; Gm5931; Slc16a7; Gstm7; Gm23997; B130055M24Rik; n-R5s205; Ssh3; Akap2;
Pigu; Cdca8; Gm7820; Gm26880; Ripply3; LOC100862267; Srebf2; Gm19868; Tmsb10;
Gm16984; Fabp1; Gm11408; Sdha; Ep400; Enox2; Rell1; Slc5a9; Trmu; Gm7160; Ebf4;
Gm14308; Zfp768; Gm16383; Frem1; Pet117; Scnn1b; Gm12017; Zfp937; Crk; Slc39a6;
Gm27219; Hook1; Rabep1; Gm10327; Mib1; Lrrc71; Epn3; Mir6982; Mir6412; Gm12508;
Gm17132; Necap2; Cacna1d; Slco2a1; Gsr; Tmod3; Slc44a3; Gm7613; Ighv1-73; n-R5s98;
Zfp781; Gm22067; H2-DMa; Gm22063; Fth1; Gm24475; Slc30a7; Uqcc1; Gm4398; Sharpin;
Dnttip2; Dnajc19-ps; Ing2; Sphk2; Acads; Gm15920; Plxnb1; Upf3a; n-R5s89; Rab3ip; Syncrip;
Gm16588; Park2; Gga3; Lmcd1; Gnptab; Gm11473; Gm24770; Ash1l; Dmtn; Hmga2-ps1;
Zkscan7; Gm6887; Gm22685; Iigp1; Cox20; Tesk2; Zc3h3; Brms1l; Asna1; Gm9824; Pan2;
Gm23722; Zeb2os; Gm12466; Gm6206; Rest; Zswim4; Mir6924; Gm5139; Gm15027;
Gm14536; Ift122; Gsk3b; Gm6184; Ezr; Gm7251; Gm23377; Spats21; Rps13-ps5; Akr1d1;
Gm23731; Zfp260; Atrx; Rbm34; Ugt2b5; Mob4; n-R5s88; Itgb5; Tsc22d1; Mir1935; Tspan1;
Cers2; 4430402I18Rik; Rreb1; Clta; Tmem231; Erbb3; Slc15a4; Thnsl1; Cndp1;
1810022K09Rik; Phf10; Adat3; Gm3839; Gm23298; Mageh1; Fam199x; Spi1; Simc1; Gm12129;
Axin1; Mir3472; Mir3472; Gm9386; Lin37; Gm22122; Gm24456; Borcs8; Gm20274; Cep120;
Far2os1; Gm9292; Gm11630; Omp; Gm15105; Timm8b; Cand2; Rgma; Txndc9; Gm13632;
Ppp2r5d; Sh3glb1; Chrac1; Rbm18; Gm22273; Gpt2; Gm20430; Mia3; Tmem86a; Dgcr2;
Gm12340; Col15a1; D2Wsu81e; Zfp512b; Myg1; Gm8624; Rpl13-ps1; Ggnbp2; Fgfr1;
Gm23079; Gm15781; Gm23355; Clec2d; Irf7; Lncpint; Akr1a1; Gm19601; Mir5128; Fam19a5;
Mir6943; Fga; Smg9; S100a2; Fubp3; Dctn4; Wfdc16; Rhbdf1; Zkscan6; Slc50a1; Tbc1d17;
Tbc1d25; Gm25633; 9030624G23Rik; Gm6807; Slc19a2; Vps41; Pcnx; Lims2; Narf; Gm9392;
Gm16056; Mir7226; Mafg; Gm11957; Gm14734; Rhoq; Spata31d1b; Rn28s1; Col27a1; Atp5o;
Emc1; Mlh3; Pnpt1; Mpp5; Gm16017; Iah1; Cdk2; Lancl1; Scmh1; Naa60; n-R5s50; Plekha6;
Gm684; Snx7; Kif2a; Dirc2; Pou5f2; Gm13461; 2810002D19Rik; Mcm4; 5530601H04Rik;
Inpp5d; Nrbp2; Gm23563; 9230114K14Rik; Cdca71; Gm26378; Flii; Nmrk1; Gm20506; Mtfr1;
Nckipsd; Carhsp1; Mphosph10; Map4; Xiap; Nab2; Rbm19; Enpp1; Vasn; RP24-414G20.1; Ddt;
Gm10048; Gm13552; Ubxn2b; Smarcc1; Il27ra; n-R5s151; Gpatch8; Rps13-ps4; Fastkd2;
Grcc10; Zfp422; Prkaa2; Zfp788; Foxb2; Tchhl1; Crot; Gm11370; Gm11643; Jund; Gm23153;
Rnf34; Gm24204; Gm13050; Myo1e; Gm26622; Ube2d1; Fam192a; Olfr558; Gm24893;
Gnpnat1; Crbn; Hacd1; Scfd1; Rcor3; Arel1; Mir1224; Ube2g1; 2500002B13Rik; Cenpc1;
Gm12109; Gm10012; Ggn; Gm10778; Gm25698; Kars; Casp3; Rplp0; Gm11709; Ldhb; Rhox4d;
Rgl3; Rnf41; Pira1; Armcx4; Chaf1a; Mrpl37; Hdac3; Timp3; Trap1; Rab4a; Gm11360;
9330151L19Rik; Gm9909; G2e3; Ppp1r8; Rps11; Smad4; Acly; Dnttip1; Scpep1; mt-Tg; Pdik1l;
Rnf19a; Gm8034; Palld; Gm20045; Phrf1; LOC100861983; Mtmr11; Tbc1d8; Lrp3;
LOC100862215; Gnrh1; Mfsd12; BC005624; Akirin1; 2210016F16Rik; Eaf2; Narfl; Gm23713;
Gm22615; Gm6098; Sdad1; Slc2a2; Gon4l; Lnx2; Faap20; Epm2aip1; Fam102b; Kcnj12; Lmf2;
Gm5698; Gm7863; Gm11771; Rfx2; Ptpn2; Gnaz; Mss51; Gm23801; Gm24485; Gm24331;
Atg4a; Gm19304; Fpgs; Btf3; Gm2383; Fbxo18; LOC100861675; 4930430F08Rik; Zfp160;
Mfsd5; Chka; LOC100503176; Txnl1; Gm15263; Hs1bp3; Fam83h; Gm15542; Slk; Galm; Lztr1;
Cetn3; Gm26528; Gm7418; Zfp385b; Gm26204; Tmem150a; Flrt1; 1110038F14Rik; Gm4853;
Wdr59; 3110040N11Rik; Rnf144a; Gm15547; Metazoa_SRP; Abcc10; Tango2; Rtf1;
2210013O21Rik; Gm17383; Atf6; Car3; Pcbp4; Wdr6; Zfp790; Cml5; Gm2a; Ccnl1; Gm12286;
Gm26285; Hars2; Tns3; Syde1; Gm13292; 4930597O21Rik; Pofut2; Rhot1; Gm5814; Kctd10;
Rnu3a; Gm11185; Cbfb; Gm13180; Tmem51; Taf4a; Snrpb; Nus1; Gstm3; Gm25312; Gm5436;
Gsg2; Tmem184b; Dpf3; Pdgfd; Zfp947; Camsap2; Gm9174; Hsf1; Depdc7; Pus10; Cdkl1;
Abcb1b; Dnajc2; Acyp2; Nudt5; Gm23037; Gm23726; Adck2; Zfp595; Casz1; Gm18737;
Epb41l4b; Tmem178; Grwd1; Pdpn; Gm15913; LOC432823; Aes; Fam53c; Zfp81; Golm1;
Gfod2; Gm11801; Tcf21; Klhdc8a; Smu1; Tmlhe; H2-Q8; Ict1; Gm23523; Gm15755; Cml1;
Mir7078; Pet100; Shpk; Slco2b1; Trappc4; Parva; Mms19; Tesc; Dll4; Atp6v1a; Rmi2;
Gm16464; Dgcr14; Pard6a; Gm15067; Snrpa; Gm22426; Rps8-ps2; Ppp1r1a; Gm22662; Pstpip2;
Gm14238; Hba-a2; Gm25801; Pex11a; Hsd17b4; Cubn; Agpat3; 1110004F10Rik; Ranbp6;
Gm24230; AU021092; Gm14414; Mir6337; Lenep; Gm24730; Prmt7; Gm24018; Cib1; Wscd2;
Gm15655; Tnrc6b; Tada3; Gm26913; Npas2; Bcl6b; Katnb1; Zfp563; Gm15785; Pola2; Rps27rt;
Gm23191; Limd1; Lcmt1; Zranb2; Entpd4; Gm16339; Slc6a8; Srp19; Mab21l3; Hsf2;
E130116L18Rik; Fam168a; Cldnd2; Alkbh6; Bscl2; Jak3; Dctn6; Tmem243; Fcer1g; Azin1;
Arid1a; Grb7; Gm3222; Pmepa1; Gm4242; Gm16073; Med29; Dhtkd1; Supt5; Mrps33; Ndufs7;
Ifnar1; Sema5a; Repin1; Rcor3; Nsun4; A330017A19Rik; Exoc7; Bcl9; Ikzf2; Gm6630;
Atxn7l3b; Gm25670; Gm5597; Dkk3; Nono; Psmd9; Gm14263; Gm4945; Adgra3; Krtap10-4;
Gm22720; Timm8a1; Serpinf2; Tmem129; Prkab1; Mettl3; Fam134c; Fut9; Fdx1l; Tox4;
Gm4262; Prkcsh; Slc16a9; 4930502E09Rik; Rps6kb1; Gm22663; Gm23391; Cyr61; Gm25580;
Sgsh; Fhod3; Srsf1; Cln6; Gm4899; Ube2e3; Rad17; Zfp426; Gm20324; Pacs1; Gpr17; Slc9a3r1;
Gm19402; Plekhh1; Suv420h2; Dcaf10; Inpp5k; Pnp2; Sun1; Mir3473f; LOC100861632;
5830417I10Rik; Gm9525; A530017D24Rik; Pld4; St3gal2; Fam126b; Gm12447; Gm3671;
Mir194-1; Gtf2i; Sfr1; Pdcd4; Erlin2; Wnt9a; Tcf20; Nae1; Slc5a8; Cdk18; Zfp511; Hpgd; Bre;
Igkv4-54; Gm9432; Gm27702; Eif2d; Gpr31b; Snord69; LOC100862155; D430042O09Rik;
Tgif1; Tusc2; C1qtnf9; Gm10540; Sumo1; Gm11560; Gm19924; Gm5944; Zfp687; Gm23840;
Det1; Zfp738; Arfgef1; Gm26397; Nfkbiz; Slc17a5; Snx30; Gm23148; Gm13400; Gm14567;
Gm25190; Ints4; Msantd3; Plppr2; Gm15653; Gm15483; Nploc4; Gm19899; Cybb; Camta1;
Ece1; Ndst1; Gm15724; Fam110c; H2-T10; Stab1; LOC100862054; Tpp2; Rpl3; Mir6360;
Rpl10; Gpa33; Actg1; Hist1h2ai; Ppp6r1; Ado; Pafah1b3; Gm10293; Gatad2b; Dgkb; Gm26983;
Srsf6; 3830406C13Rik; Nphp1; Rpl28-ps1; Pex11b; Amigo1; Ufsp2; Alg12; Gm15504; Fibin;
Gm19816; Gm25321; Gm8659; Npepl1; Oraov1; Nars; BC026585; Galnt1; Mvp; Gm11307;
Efna1; Eif3l; Bcl9l; Gm11787; Zfp319; G530011O06Rik; Gm24189; Slc22a6; Zfp58; Mtx2;
Mir6916; Gm23721; Hgfac; Klhl6; Ei24; Lgr4; Gm26600; Hnrnpc; Fam204a; Hoxc10; Tm2d3;
Fahd2a; Gstt2; Tubb2a; Rfc4; Gm5708; Osbpl10; n-R5s51; Rfwd2; Gm12110; Gm25193;
Ppp4r1; Smt3h1-ps; Pym1; Gm24143; Gm22395; Ighv1-53; Fbxo7; Gm53; Sema4g; Zfp438;
Mtch1; Prrc2c; Gm27445; n-R5s21; Cyb5d1; Fbrsl1; Ccdc3; Gm2991; Gm13755; Rufy2;
A930015D03Rik; Mcm2; Gm7676; D8Ertd82e; Id3; Tuba1a; Scnn1g; Fam160a2; Krtcap2;
Gm4013; Patl1; Gm12959; Gm19636; Gm15530; Cep83os; Zfp704; Phtf1os; Gm25798; Trim25;
Gm24968; Tnfsfm13; Ptdss1; Osbpl3; Gm11950; Sars; Frmd4b; Olfr527; Gm4294; Pex6; Adra1a;
Mbnl2; Lama4; Gm14584; Gm24140; Mir7004; Gm2115; Trp53rka; LOC100862335; Gm24339;
Rpl9-ps4; Gm8752; Gm23833; Anapc2; H2-DMb2; Gm10011; Dnajb6; Gm22494; Hmox2;
Trbv2; Aldh6a1; Acacb; Gm10250; Gm6285; Arf2; Gjb4; Gm9951; Gm2546; Gm13146; Zfp282;
Tmem19; Nr2f2; 1700015O11Rik; Gm16330; Mterf3; Nup98; 2810408A11Rik; P2rx7; Gna11;
Gm13111; BC017643; Gm24592; Rab11fip4; Prep; Usp31; Atp5d; Pold4; Gm11380;
3000002C10Rik; Cfp; Iyd; Sez6; Sec16a; Hsd11b2; Elac2; Orai3; Kmt2d; Fastk; Gle1; Acer2;
Yeats2; Gm11271; Rgp1; Gm14193; Herc4; Xbp1; Mtrf1l; Peo1; Zfp964; Atxn2l; Taf5l;
Gm7117; Sde2; Gm12070; Fbxo11; Pabpc1; Eea1; Tardbp; Kcnc4; Gm26647; Pygb; Gm5939;
Sh3d19; Zmiz2; Mpv17; Gm23918; Snord123; Arl2bp; Slmo2; Ankrd12; Zfp182; Usp53; Ppm1d;
Gm6750; Rpl9-ps7; Crb3; Shisa5; Tsn; Gm24826; Poglut1; Gm13431; Atp5b; Ctc1; Hp1bp3;
Ccdc25; Gm8343; Ifit2; 4930526F13Rik; Eif2s2; Slc52a2; Aprt; Cfhr2; Tmem43; Gm12712;
Gm22655; Gm12828; D5Ertd605e; Efna5; Ralgapa2; Top1mt; Gm13652; Ppp5c; Vps4a;
Gm9507; Fzr1; Rmdn1; Gm25672; Numb; Sap25; Gm9457; Pigc; Uhmk1; Rpp25l; Tfap2b;
4930555K19Rik; Gm26783; Gm22251; Tprn; Trp53cor1; Alg9; Grhl2; Slc39a14; Gm11997;
H2afx; Sec24b; Gm12987; Nup188; Izumo4; Fndc3b; Gm7572; Adgrg5; Idh3b; Noc2l;
Gm26328; Specc1l; Gm14497; Gm25777; Prdm2; Cpsf3; Snora41; Hoxb8; Ints3; Tmem151b;
Eapp; Bap1; Gm4835; Tssk3; Eif3k; Agpat2; Eif2ak1; Armc8; Gm24767; Gm8709; Alms1-ps1;
Gm26023; H2-Aa; Igtp; Fam234b; Khk; Gm13378; Fads3; Taf6; A230046K03Rik; Chrna7;
Lamc1; Cc2d1a; Tmem114; Rrp1; Cutc; Rnf181; Gm12178; Mgam; Btbd10; Ppara; Gramd3;
Gm25554; Fam63b; Mir322; Nudt2; Cbr2; Wdr1; Helb; Ppp1r7; Gdf15; Scnm1; Pcbd2;
2510009E07Rik; Gm8532; Gm15824; Phtf2; LOC100862001; Ptger3; Casp7; Mettl2; Fam53b;
Elovl5; Pgap3; Acsl5; Pdp2; Mrpl17; Gm15375; Raph1; Por; Gm11710; Stard10; Aamp;
Gm13171; Ercc3; B130024G19Rik; Gm25442; Osbpl11; Gm13039; Gm11222; Slc35e2; Dnaja1-
ps; Scarna6; Gm24272; Rsph9; Hivep1; Add1; Gm16195; Lats2; Gm6563; Nfkbia; Gm10131;
Surf1; Jun; Elmsan1; Bid; Renbp; Txndc5; Slc2a9; Pf4; Ptges3; Commd8; Pebp1; Cxxc5; Ehf;
Fam160b2; Cacnb4; Smc3; Cox6b2; Gm15528; Tuba1b; Rcl1; Xylt2; Ilf3; Hbp1; Rbbp4; F2r;
Cox6a1; Gm26272; Gm22043; Igkv2-109; Gm13181; Nos1ap; Rnf170; Gm4609; Eif3e; Traj29;
Kat6a; Rny1; Polr3b; Gm20346; Tmem189; Gm8988; Gm25844; Gm16315; Gstt1; Poc5; Ints6;
Mlxip; Gm12551; Saraf; Larp1; Gm14008; Gm8864; Arpc4; Xpo1; Pdcd6ip; Rac2; Cxcl12;
Gm14130; Svip; Irgm1; E330033B04Rik; Slc10a7; Gm19486; Ppp1r14a; Gm5652; Fgfr3; Clpb;
Cox15; Gm14719; Gm24756; Fam208b; Gm7985; Gm22994; Gm8292; Bsdc1; Gm22637;
Gm17402; Dhx30; Gm19865; Cdkal1; Stag1; Gm14777; Secisbp2; Abcc1; Ehd2; Bbs9;
D630036G22Rik; Dnmbp; Gm11991; Traj25; Ifrd2; Top3a; Ppp4r2; n-R5s161; Gtf2b;
4930578I06Rik; Bag4; Alox5ap; Gm5617; Pik3c2g; AA986860; Orai1; 0610040J01Rik;
Gm9844; Sh3bgr; Plekha7; Sgcb; Fyttd1; Fabp5l2; Cald1; Fgf1; Gm22944; Mdfic; Selm;
Gm25537; Gm12983; Mcm3; Lctl; Gm25117; Kif21a; Cyp1b1; Qprt; Gm26323; Lamtor1;
Mir3098; Pex19; Gm13464; n-R5s28; Zfp42; Itpa; Sap30; Gm13509; Hspa4l; Rab11fip2;
Gm11509; D930048N14Rik; Ctdspl; Gm13197; Gmpr; Cfi; Scp2-ps2; Snx9; Anks6; Slc35e4;
Gm4654; Gm9825; Tmem179b; Efr3a; Lmntd2; Trip11; Tomm6; Hsd3b3; Gm20536; Plxna2;
Gm11214; Gm17113; Mpzl1; 4931414P19Rik; Hid1; Gm12348; Tbc1d20; Ranbp10; Gm5899;
Dnajc1; Pcdh17; Ikzf4; Dse; Gm19774; Wdr19; Tbc1d15; 4930402H24Rik; Med1; Rny1;
Bmp2k; Gm22567; Zfp74; Gm7129; Atad3a; Mir6962; Gm7816; Mitf; Gm4795; Gm15859;
Tmem104; Eif3h; Gm13545; Pop1; Ankrd13b; Gm22113; Wbp1; Cyth4; Gm3200; Fra10ac1;
Gpr4; Dmgdh; Stard3; Slc17al; Stx6; Gm12866; Pip4k2c; Orc3; Gm15848; Cad; Gm12523;
Gm7879; Veph1; Gm11747; Gm19794; Ssr2; Ndufaf3; Gm27530; Lacc1; Rnf43; Slc9a7; Ovca2;
Tspan14; Entpd2; Ctdsp1; Afg3l2; Fcgr2b; B230104I21Rik; Gm5313; Gm10418; Lima1;
Gm12482; Gm13340; Ninj1; Gm24596; Gm25591; Irx3; BC021891; Gm25414; Gm5108; Agbl5;
Coro2b; Tnfrsf22; Ugt2b38; Pnpla6; Gm22508; Hist1h2ao; Nfkbie; Gm17304; Klkb1; Ighv1-11;
Kalrn; Lrrc61; 4930526A20Rik; Xaf1; Gm23437; Plagl1; Esrrb; Gm26308; Ptov1; Nudt12;
Eif2b2; Aspdh; Egr4; Setd1a; Mir6965; Gm10013; Grik5; Gucy1b3; Tmem246; Polr2f; Ank3;
Gm15519; Slc12a9; Zdhhc6; Tbcb; Olfr686; Ggps1; Gm13810; P3h1; 1810032O08Rik; Egfl8;
Zdhhc20; Gm12481; Gm20157; Lrfn3; Arfip2; Atpif1; Smarcb1; 2210016L21Rik; Anapc11;
Pdpr; Gm3555; Dhrs7; Gm24290; Nsun3; Shroom3; Hyal1; Arhgef5; Magt1; Gm22761; Sdccag3;
4930466I24Rik; Gm15198; Hccs; Fam102a; Slc45a4; Map3k9; Vamp7; Ifi27l2a; Pdzk1ip1; Gp2;
1700044C05Rik; B4galt3; Gm27405; Mterf1b; Kifc3; Slc48a1; Cul7; Qrsl1; Dusp9;
A330023F24Rik; mt-Tf; Rab3a; Igkv8-27; Adipor1; Pomt1; Sim1; Gm12220; Ube2q1; Rhox4g;
Cyp51; Mfap3; Cdk12; Gm4804; Hmx2; Bola2; Ints8; Dync2h1; Gm14702; Gm15793; Zcchc8;
Adrb1; Pnrc2; Sugp1; Mir3065; Clca3a1; Lonp1; Gm24824; Slc6a3; Dus2; Cdkl2; Gm26999;
Ndufb7; Dgkq; Gm15583; Ube2w; Bcl2l1; Ildr1; Gm10181; Nqo1; LOC636187; Phf6; Net1;
Gm4788; Rhox2f; Gm24054; Ppp1r2-ps3; Ppp4r1l-ps; Znrf3; Flnb; Atp1a2; Cstf2; Sec24c;
Gm24870; Arfip1; Chst14; Gm19705; Lipo2; Immp1l; Gm11450; 3-Mar; Zfp748; Ube2i; Erf;
Tomm70a; Esm1; Max; Tal1; Foxp1; Cdt1; Rnf26; Olfm4; Gm13369; Igfbp4; Scaf4; Agps;
Fam120c; Isoc1; Fa2h; Gm20548; Tmem30a; Gm14964; Gm7931; Atp1a1; Slc6a7; Gm12117;
Rpl37rt; LOC100862227; Hmgxb4; Cpsf7; Eef1g; Ggt6; Gm15753; Gm17080; Trav4-3; Aqp11;
Desi2; Hist3h2bb-ps; Gm11518; 2810025M15Rik; Dusp12; Nme3; Pdzd3; Gm20185; Mir6913;
Gm24445; Gm20454; Exosc4; Lpar3; Clec16a; Tug1; Mir188; Acss1; BC017158; Mrap; n-
R5s197; Gm24530; Gm15934; Zfp574; Pgam1-ps1; HOTTIP_1; Gm9988; Puf60; Gm15985;
Eif4a1; Rnf6; Rel; Gm12260; Zfp532; Prrc2a; Ighv2-1; Gm13185; Nckap5; Zfp770; Gm23368;
Mrps2; Tnpo1; Tnnc2; Mir7-1; Zkscan3; Crtc3; Tcea3; Asl; Papd4; Sec14l1; Tax1bp1;
C030037D09Rik; Gm12957; Syvn1; Lgals3bp; Gm20594; Slc10a5; Gm23279; Abca7; Gm23207;
Trbv14; Med13l; Wars; Prpf18; Pcmtd1; Rps10-ps1; Dcun1d4; Gm25245; Asb13; Gm10275;
Fam173b; Ube2ql1; Hdac2; Mir874; Taf1b; Ssrp1; Dhx34; Jam3; Polr2b; Acp2; Cstb; Gm23884;
Cpsf3l; Bckdhb; Gm22666; Noc3l; Mapk1ip1; Snx2; Skap2; Nudt21; Zscan22; Dnaja2; Rpl9-ps1;
Gm12013; Ndufa9; Ppp2r3d; Rex2; Gm14276; Tbce; Cln3; Gm26047; Ccdc68; Rap2a;
LOC100503407; Gm16232; Gm9396; Ighv5-12-4; Cdip1; Olfr523; Gm8121; Adra2b; Gm7125;
Gm5514; Tmem131; Slc35f2; Foxp4; Tagap1; Gmps; Gm16505; Gm13131; Pbld2; Ap3s1-ps2;
Rpl12; Csgalnact2; Phf1; Rap1gap2; Taco1; Tesk1; Gm27012; 0610010K14Rik; Gm24514;
Fam163a; 4930515G01Rik; Gltscr1l; Gm12751; Nipbl; Cobl; Uchl1; Arhgap26; Hist1h2bc;
Slc22a18; AB041806; Per1; Emilin1; Has2; Gm9797; Mex3c; Tomm5; Sqle; Bptf; Col5a2; Pak4;
Tex9; Gm22464; BC023202; Gm13649; LOC100862164; Trav13-2; Glyat; Gm5909; Gm22701;
Ssh1; 2310057M21Rik; Kcnj15; Ccdc71l; Gm5185; Gm12536; Pla2g6; Gm24362; Aak1; Clpp;
Gm7502; Fbxw4; Mchr1; Msgn1; Mrps30; Hfe; LOC100861604; Hps1; Sgsm3; Gm5523; Exoc8;
Gm14429; Ssfa2; Ttc13; Echdc1; Mief1; Ppm1a; Nudt9; Map3k8; Stx12; Itih5; Gm20451;
Cyp4f37; Gm25092; Myo5b; Ppp2r1b; Gm15148; Clrn3; Kctd13; Gm12428; Acaa1a; Tubb5;
Ttpa; Mir7036; Gm11953; Calcrl; Gm12176; Bri3; Gm26180; Arhgdib; Gm14190; P2rx4;
Gm26122; Mtmr12; Msi2; Ablim2; Gm22286; Dennd3; Gm22120; Gm23045; Tor2a; Gm15458;
1700031F10Rik; Gm15696; Gm23168; Gm27392; Gm12895; C1galt1; Uchl4; Dazap2;
Gm25847; Fam210b; Atp6ap2; Rad54l2; Wbp1l; Gm10313; Zscan5b; Gm13343; Gtf3a; Sypl2;
Rpl26-ps5; Mir1951; Me3; Ddx31; Phf8; A730035I17Rik; Gm19725; Gm25201; Il17re;
Gm10931; Abhd17b; Mapk1ip1l; Gm24535; Rorc; Gm24644; Ick; 6030426L16Rik; Gm25618;
Gm7206; Dtx3; LOC100861902; Gm6736; Tsga10ip; Gm23540; Ppp2r5b; Ptger4; Thoc7;
Gm5451; Tbcd; Gm15481; Gm24996; Abhd12; Urb2; Mipep-ps; Ddx59; Rhpn2; Pspc1; Snhg3;
Atp11c; Lypd5; Gm14209; Samd4b; Gm8357; Aamdc; Psmd11; Mpzl2; Gm13339; Npnt; Cops8;
Proca1; Gm8662; Taf1a; Afm; Sft2d2; Aktip; Gc; 9130230N09Rik; Pfdn4; Tefm; Gm16145;
0610030E20Rik; Gm12161; 2700046G09Rik; Gm24145; B330016D10Rik; Gm7068; Gm21814;
Stk25; Ccdc50-ps; Mrps34; Sf3a1; Gm4217; Bbip1; Gm19377; Cfl1; Pcp4; Spred1; Foxo3;
Gm2862; Robo2; Gm11438; Gm8839; Ppdpf; Gm11472; Med12; Ost4; Lsr; Slc33a1; Ogdhl;
Gm22201; Vax1; Gpn3; Gm12054; Gm6104; Coq6; Fam134b; Gm26442; Tgoln1; Ces2h;
Eif2ak3; Gm14024; Abhd14b; Pde4a; Layn; Nolc1; Mars; Gm22057; Ttll4; Polr3e; Mir6934;
Actr8; Htatsf1; Gm4929; Pomgnt2; Bckdha; Eps8; Gm16437; Gm20554; Gm12258; Gm26582;
Nek9; Gm27974; Cd47; Ndufa12; Gm7664; Gm6478; Gm12235; Sik2; Fbxw8; Ankfy1;
Gm25770; Inpp5e; Smim11; Gm7887; Gm17163; Gm10734; Mir6960; Hscb; Gm22443; Cideb;
Pianp; Arhgap29; LOC432823; Abi1; Upf1; Gtf2e2; Gm12791; Mir7023; Dip2b; Slc2a5;
Gm13965; Daglb; Gm12307; Gm10025; Gm8100; Cyp2c44; Otud3; Gm20760; Tubb4a;
Dnajc30; Pla2g3; Gm20721; Pemt; Cdk2ap1; Ralb; Arhgap8; 4930553M12Rik; Tarm1;
Gm10012; Ly6i; Acot11; Gm15331; Gm14231; H2-Ea-ps; Smarce1; Spn; Gstz1; Dnajc19;
Ociad1; Rpl15-ps2; Tssk6; Ccdc153; Rab7; B930078G14Rik; Gm19658; Commd10; Bcam;
Mir467c; Rpl18a; Tubgcp2; Tmem167; Mir5115; Gm15013; Igip; Gm12123; Mtdh; Rnmtl1;
Gm23388; Cacna1i; Degs1; Rhbdf2; Cnnm3; Gm23199; Mir7665; Prodh; Srcap; Zdhhc14; Slx4;
Caly; Gm14967; Gm26211; Mbd1; Rpl10-ps2; Gm24284; Rasgrp3; Abhd2; Mir7046; Tdp1;
Abcd4; Gm14472; Alg11; Emc3; Mt2; Herpud2; Cplx3; Dtx4; Sbf2; Dcp1a; Pbld1; Atxn7l1;
Atp5l-ps2; Gm11483; Gm19246; Mir6361; Bbs2; Lrsam1; Cst3; Gm10822; Rabif; Gm8730;
Vat1; Gm17233; Gm14639; Iars; 2410015M20Rik; Angel2; Metazoa_SRP; Slc25a22; Mir8105;
Pcbp3; Gm20379; Spg21; Slit3; Ap1g2; Sar1b; Ap3s1; Zbtb46; Gm6180; Wac; Mov10; Gkap1;
Lmbr1l; Nfkb1; Limch1; Sgk2; Prnd; Zfp598; Gm8756; Gm24326; Snrk; Pcsk6; Gm26318;
Kcnj1; Gm12416; 0610007P14Rik; Gm5787; Mir8103; Zbtb7c; Phf11a; Them6; Gm2745;
Wdr75; mt-Th; Slc25a11; Ccdc84; Hcfc2; Gm23969; 6230400D17Rik; Ctps; Gm25173;
Gm25211; Gm26324; Gm23732; Cbs; Tbc1d10b; Psmc5; Sult1a1; Vps53; Gm9105;
3110002H16Rik; Gm21857; Pik3c3; Cul1; Gyltl1b; Gm17374; Myo19; Gm21103; Fbxo33; C1s2;
Supt6; Dnajb14; Gm13488; Cebpg; Gm23706; Gm10291; Mapk8ip1; Parp8; Frs3; Hoxd8;
Tmem115; Tulp4; Spa17; Gm10221; Srsf3; Stx2; Prrg4; Trappc11; Smchd1; Pigs; Cxx1a;
9030619P08Rik; Bach2; Atg4c; Cox7b; Gm11688; Fzd5; Hspa13; 5330438D12Rik; Klhl29;
Syngr1; Ddrgk1; Aebp2; Gm26127; Tcerg1; B630019K06Rik; Rpe; Gm14853; Rpn2; Mir3060;
Zdhhc3; Mepce; Gm27306; Ighv2-3; Mettl1; Gm13765; Atp2b4; Lin7a; Gm23755; Snapc1;
Gm25319; Nlgn2; Igkv4-59; Coq7; Ell; Gm11979; Rrm1; Ctdp1; Cdkn2aipnl; Cnksr1; Gm22993;
Gm5255; C730027H18Rik; Tmem14c; Tmem26; Drap1; Coq9; Tmem161a; Rbck1; Blmh;
Slc6a13; Epor; Gm25125; Fau; Hrct1; Dhx32; Zbtb39; Igkv3-2; D1Ertd622e; Coa6; Fryl; Irak1;
Gbp10; Prkcq; Ptpn4; Rhobtb1; Numbl; Gm8974; Gm25635; Snapc4; Gm13680; Gm25660;
Gm11453; Gm5687; Lrp2; Sirt7; Cap2; Gm24032; Ubxn2a; Gm22304; Rps27-ps1; Zcchc17;
Zfp280c; Adamtsl2; Gm15714; Mir196a-1; Amdhd2; Mir6959; Abcc6; Mir1935; Mir7047; Ocrl;
Samd8; Hsd3b6; Txnl4b; Zswim8; 1700105P06Rik; Mir1195; Dctn1; Mir7003; Gm13392;
Gm10653; Hoxb3os; Fam84b; Arl6; Extl3; Gm16298; Rab34; Gm6091; Gpc2; Mir1957a;
Hmgb2; Wipf1; Slc35f1; Nf2; Gm16096; Ilvbl; Mxra7; Gm28003; Cgnl1; Irf3; Mirlet7e;
Gm7670; Tsg101; Gm23657; Wasl; Myo1g; Bach1; Tbc1d32; Tnfrsf21; Jade2; 1010001B22Rik;
Gm15475; 1700028B04Rik; Cryl1; Gm15766; Grb10; Syne1; Gm26490; Amn; Tubgcp3;
Gm22926; Usp15; Mat2b; Gm10780; Lrp4; Cyb5r4; Gm7847; Gm22886; Nudt4; Ttc37;
2500004C02Rik; Steap4; Gm28033; Hist1h2bj; 0610011F06Rik; Gm11773; Dtnb; Gm22355;
Gm26168; Gm5763; Atp6v0a2; Gm12396; Rny3; Rny3; Snrnp35; Gm10355; Gm8199;
Gm13410; SMAD5-AS1_4; Gm13424; Tti1; Gm14091; Mir7661; Plbd1; Kat5; 2410002F23Rik;
Tmod1; Rn18s; Sh3glb2; Alpk1; Gm24748; Gm23322; LOC100861967; Aph1a; Bhmt2;
Gm19426; Pik3c2a; A730008H23Rik; Snora69; Naif1; Satb2; Gm25516; Thoc1; Pex26; Pik3ap1;
Cldn1; n-R5s168; Gm12240; Zfp512; Atp6v0a4; Commd4; Anks4b; Gm12490; D730003I15Rik;
Praf2; Gnb2l1; Sppl3; Gm17541; Rcan3; Mir684-1; Skiv2l2; Cnn3; Cct4; Grb14; Vps13d;
1810012K16Rik; Srf; Trav4n-3; Egf; Sfrp1; Gm7507; Mbip; Fam188b; Enox1; Gm15567; Pdk1;
Tnnt2; Ltn1; 2310040G24Rik; C1s1; Gm23973; Gm8174; Zfp335; Gltscr2; Prmt5; Raf1; Igfbp5;
Gm7493; Gm13737; Acot2; Gm25101; Mdp1; Gm27284; Gm7781; Srsf10; Colgalt2; Rdh14;
Gm26189; Vcp-rs; Qrich1; Thap2; Eif5; Abca13; D930016D06Rik; Gm12138; Gm6594; Amotl2;
Gm24464; Arhgap5; Gm7609; Gm22352; Gm24584; Mir143hg; Hlcs; Ptprj; Atp6v1f; Gm10322;
Igflr1; Asun; Lrrc41; Egfl7; Evc; Atp10b; Fam107b; Gm12693; Arfgap3; Dusp4; Aaas; Prkaca;
Aspa; Gm7809; Cldn3; Mex3a; Ptgds; Traj46; Gm9994; Erbb4; Kl; Col6a1; Gm15195; Cyp3a13;
Gm25665; Ccl5; Zdhhc23; Rnf146; Slc22a2; Mcmbp; Neo1; Fam179b; Colec12; Arhgap10; Siae;
Gm23712; Dopey2; 2610037D02Rik; Syn2; Timm13; Brpf3; Tmbim4; Cdc25a; Arfgap1;
Bloc1s2-ps; Rpl31-ps6; Snrnp48; Mob2; Tor3a; Nkiras1; Cpeb4; Hmgn2-ps1; Wdr41; Gnl3; H2-
K2; Cnrip1; Yae1d1; Gm27529; Cct2; Cdh22; Pde8a; Gm15454; Rab3gap2; Slc41a3; Tbc1d24;
Ndufaf5; Eif4g3; Otulin; Pvr; Olfr239; Gm24265; Gm4819; Ncr3-ps; Gm10184; Prrg3; Trip6;
Tomm34; Slc25a30; Mgat1; Gm26808; Gm13357; Gm23965; Rab5a; Rps15a; Rtn4ip1; Flcn;
Ctla2a; Rsbn1l; Sav1; C030039L03Rik; Dip2a; Emc2; Vwa8; Parm1; 4933412E12Rik; Fgd3;
Mbd6; Ptgs1; Gm26476; Mir684-2; Nol12; Dkc1; Slc13a1; St7l; Lcor; Gm16238; Phc2;

Tables 26-1 to 26-60: MEGENA gen2 Modules: NZM2328 Glomerulus
The genes in each of Tables 26-1 to 26-60 can be used as effective biomarkers for classifying the LN disease state of the patient.

TABLE 26-1
Module 2.1

KLK1, EMP3, C1RB, P2RY6, EFHD2, MGAT5B, FRZB, TGIF1, MMP14, ARHGAP5,
CAPG, IFI30, TCIRG1, CSTB, GNAI2, DPP4, THBS1, PLK3, GADD45B, LRBA,
ARPC1B, GRN, DUSP5, BHLHE40, RAB32, BOK, LRRC1, ACTA2, LGALS1, RUNX1,
CD14, GLCCI1, RMND5A, ENPEP, RPLP0, MYADM, ATF4, ABCA1, IFNGR2, RAB31,
CDC42EP3, NEO1, RNF19B, CXCR4, ABCB7, SYNGR2, REEP5, GSTM2, PLD3, MYC,
ZYX, NUAK1, SBNO2, PGS1, FSTL1, MAP3K8, PRKAR2B, PF4, SERPING1,
LGALS3BP, C1S1, AIFM3, LRP6, NFKBIA, CKAP4, AP2S1, NFKBIB, MFSD5, RASSF5,
PRR13, AKNA, IRAK2, CEP85L, LY6E, NPC1, RHOC, EGF, 2610524H06RIK,
2310022B05RIK, PPP1R14B, RASSF10, FKBP10, ITGB8, NSA2, MYO6, GNA13, TSPO,
LITAF, CX3CL1, LGALS9, CDK4, TBC1D19, FOLH1, PHLDA3, TAF9B, ALDH3B1,
TAPBP, NGF, CHPT1, EDN1, CALM1, CSRP1, TAGLN2, S100A6, GLTP, TIMM13,
SFR1, SYNE1, MUSTN1, KDR, PRKCDBP, 9130401M01RIK, CMTM3, CNN2, MFGE8,
RRAS, SERPINA1C, NUCB2, TMED10, GDF3, 9-SEP, S100G, FBLIM1, LRP10, ADAT3,
TPCN2, NTRK2, NID1, COL8A1, TEK, CAR13, PLA2G15, CGNL1, RAB20, ARMCX6,
PTGER3, EGR1, ETNK1, KIF21A, SCRN3, RB1CC1, PMF1, ABCA5, BTBD11, MVB12A,
RGS10, MGAT4A, STX11, GLB1L2, NRM, SRF, PAPPA, PRELID1, CIB1, TWIST1,
2700094K13RIK, OSBPL6, PSMC4, UACA, NUMB, MAN1C1, ARHGDIA, EPHX2,
IQGAP2, BOLA2, ITGA9, ITGB6, PCM1, NPY6R, POLR1D, FIBP, NT5C, DCAF15,
TMEM160, GSTM3, CAPZB, PRAF2, RCN3, RHBDF2, YIF1B, DEFB1, ASNA1, CELF2,
SMIM3, SLC4A9, CEBPB, SNX29, TSPAN13, SLC25A1, TNFRSF4, ARSJ, PGAM1,
PSMC5, NEXN, WDR63, RGS9, SPTBN5, MOB3A, ATF3, NNT, RPL32, PSMA5, CMBL,
UGT2B34, 1810011H11RIK, YIF1A, CCDC68, ACOT13, 1700016C15RIK, UBXN2B,
MPND, EHD3, CPEB4, SRM, CTSW, MGP, DDX28, ADAM9, BMPR1A, WWP1,
TSPAN17, ABCASA, FOS, FAM26E, EHF, BAX, CXCL14, KDELR2, MRPL2, PCMTD2,
OSBPL9, NHP2, CPB2, DDAH2, COPE, MAMLD1, ARMCX2, GM4951, ALG3, NDST3,
NTF3, ZMYM5, TTLL12, GSTM1, PANK1, TMEM64, CADM4, IFI47, RIPK3, EFEMP2,
HIGD1B, SOX18, BCL2L11, VPS37C, ZFP40, ENG, MEX3B, ATP8B1, ABCG2, STAT3,
PITPNM1, BMX, PSMB5, ZFP277, DPEP1, MMP11, PVR, CCND1, MEX3D, EDNRA,
NR4A3, PDGFD, SSR2, KLK1B5, AGAP2, CERS6, FBXO3, LPP, PON2, KIF12, SHMT2,
MAPK3, GPIHBP1, ATG101, LRRC20, COPZ1, PDIA5, ZMAT5, MFSD10, SLIT2, REN1,
ZFP266, TBCB, CREBRF, ARPC5, PTS, TNFRSF10B, ABI1, UBL7, PAQR5,
1700029J07RIK, SHANK2, TTYH2, SH2D4A, BC048403, CPT2, ARL15, PI4K2A,
NKAIN1, HLX, LIX1L, AMOT, NEK6, BOC, SNAI2, KBTBD3, MRPL36, FAM46A,
ZBTB42, KIN, AA467197, FAM104A, TMX2, FAM81A, NEDD8, C1RA, SLC35F2,
VSIG10L, JAM2, CRTAP, ARFGAP3, ZFP36L2, GM5945, FOSB, SLC16A2, TMEM138,
ENHO, UBQLN2, UQCRQ, RNF122, LGI2, OTUB1, TLDC2, MARCKSL1, IER5L,
A930007A09RIK, CHMP5, LRRC28, WDR82, SCIN, ADGRG6, SHISA5, PRG2, DAPP1,
1500009L16RIK, XPA, 9130008F23RIK, CST12, HEY1, ACSF2, GCNT4, TMEM47,
WARS, PLK2, MET, UBALD2, PAK1IP1, ARRDC4, TMEM252, RARB, ACOT2,
PLXDC1, DAPK2, SMDT1, 1810011O10RIK, ARHGEF38, SRR, SNX3, ART4,
ARHGAP26, ZDHHC4, SOWAHB, DPAGT1, IGSF5, PLCB2, 1110007C09RIK, SERF2,
GM3414, MND1, TRP53I11, SOX17, TSPAN12, OLFR110, RPRM, SELP, SOX7,
UQCRC1, OLFR649, CALML3, DEFB29, ALPL, ART3, CUEDC2, PDGFRA, STAP2,
DOPEY1, IFT22, TMEM18, DEFB23, DDAH1, 2310050C09RIK, DIO1, RASSF9, CLN3,
MCTS2, NPC1L1, CLCN7, POP4, MED10, MFNG, TUBG1, SLC35D2, POLB,
A930018P22RIK, CTSO, OXSR1, RARRES2, CDK5, CNTN1, ZBTB10, ATP10A, NSDHL,
BRE, 2210016F16RIK, WFDC10, EHBP1L1, RPS9, RNH1, SH3KBP1, NFATC1, VEPH1,
GMIP, SRSF9, BGN, C1S2, NMRK1, TBC1D2B, IMPDH2, SLC30A9, TRAFD1, CALM2,
EGR2, ZFP423, RPS26, PCGF5, PCMTD1, ARPC3, CLCN3, RNPS1, SCPEP1,
SERPINA1E, SSNA1, CCSER2, RFTN1, SNRNP25, DBN1, CDKN2AIPNL, NMT1,
OLFM4, TXNDC5, LIN7C, KIF16B, SEL1L3, CTPS, MYCBPAP, TRAF3, TRIM47,
RAB24, STARD8, CCDC115, SLC26A2, HAUS4, FAM96B, ARL4C, AIMP2, LRRC66,
SYT17, ASCL2, COPS7A, MRPL34, CDON, GTF2F2, AP1S3, DCTN3, HIGD2A, GPHN,
VPS28, CPNE3, RQCD1, TMEM38B, MKL1, HIST2H2AC, CDS1, DTNBP1, EMP1,
MYCT1, AVPI1, IFI35, UBE2L6, XRCC5, TINAGL1, TRIM8, AQP3, BOP1, ZFP612,
EIF2S2, PLEKHJ1, EIF5A, FBXO8, ETHE1, GNL3, UBTD2, GM8773, AMT, GPX7,
POLR2J, PCDHB15, SNRPC, ADAM22, NOXO1, SLC25A43, HNMT, S100A16, ASAP2,
LTV1, SLC9A5, GNA14, UNC13C, PTGER4, TMEM86B, KLHL12, MPHOSPH6,
OLFR449, DUSP23, GPR182, KLK1B27, RDX, TMEM158, RCE1, RABAC1, GM2933,
OLFR338, ZFP300, KCNIP4, TNFRSF26, GM5382, MPTX1, NOXRED1, OLFR632,

TABLE 26-2
Module 2.11

EIF3M, LFNG, CACNB3, LLGL1, APELA, ZMIZ1, CORO1C, MYO9B, FLNA, GPCPD1,
MCAM, RNPC3, GALNS, RELA, SYNJ2BP, POR, FURIN, KIZ, RHOB, CFL1, SULF2,
HMGA1, PLXNB2, FASN, EPM2AIP1, ST3GAL6, GRINA, MNDAL, GIT1, BABAM1,
3830406C13RIK, SCAMP3, PARP3, ZFP598, HIST2H2BE, VPS37A, ARRDC1, SETD1A,
ICE2, ZFP759, ZC3H12A, APOO, SGTA, PCIF1, RAB4B, RAB43, CARM1, GM4787,
MOCS2, ANKRD13A, SFXN4, TESK1, ITFG1, NAP1L1, HSPG2, TMEM259, SAMD5,
THEM6, ARL8A, DPP3, 1500011K16RIK, PIANP, TTC14, N4BP3, MPV17L2, CITED2,
EPOR, TMEM43, MKNK2, LAMC1, COX20, ENDOU, GATSL3, SEMA6B, PSTK,
CCDC173, PNPLA2, LRRC4, TUBB4B, NET1, CRY1, LMCD1, DHRS1, ROBO4, RCC2,
LOXL2, MRPL42, CEP112, TRAM2, SUPT3, FAM229B, FBXL6, TRPV4, ADAMTS1,
SAP25, RMDN1, STUB1, YME1L1, ANXA6, CMIP, IMMP1L, ADRA2C, DOK1, TSSC4,
EMCN, ITGB3BP, STFA2, GRK6, NOSTRIN, LIMK1, SHARPIN, CDK10, ACTR6,
ADAMTS9, NOP10, 2510039O18RIK, MSMP, SLC6A9, WDR1, ANKRD52, MRPS33,
FADS1, EMC2, ATF7IP, HINT3, HERPUD1, TSPAN8, INMT, REPIN1, IER3, GRAP,
MKKS, PREP, SLC2A1, KIF5B, NLGN2, PPP1R3C, RANBP6, TLN1, TNFAIP1, NEU1,
CRIP2, ARL9, TRPC6, NDUFB5, UNC5B, SMAD1, ADD2, CNTLN, E2F5, MAFB, JAG1,
ANXA2, CETN2, COX17, MCEE, SQLE, LDLR, MKRN2OS, ALDH2, GLO1, TRIP10,
IRAK4, CAAP1, 2300009A05RIK, RASL2-9, SPP1, ZFP938, ZNRF4, GM13275, CCNC,
ARHGEF37, TUBG2, CCPG1OS, BBIP1, CYGB, LZTFL1, METTL15, LMF2, EIF4E,
GM2799, MAP6, CLDN15, PXDN, PRRG1, ZNRD1, DAGLA, CCDC94, ASXL3, MYH10,
AGPAT4, C330018D20RIK, IGFALS, CLU, GPX8, GJA5, DISP1, 6720489N17RIK, HPGD,
LY96, F2RL1, CCDC167, IFRD1, PCDHB20, CAMKK1, CEP70, ND2, NAIF1, CAPSL,
AP4S1, ELK3, 1190002N15RIK, TPK1, RBBP7, TSPYL5, ID1, RAMP3, SPRY1, AP5M1,
CHST1, EGLN1, EEF2, ZFP563, IL17C, TAS2R135, GTF2H3, RNF40, TSPAN6,
OLFR522, AW209491, DESI2, STXBP1, ATP5S, KRT19, 1700001J03RIK, LRG1,
TUBB2A, MTCP1, MED16, MSMO1, DRAM2, USP27X, HAVCR1, SLC25A20, ZFP11,
AOC1, 4930544G11RIK, ROBO1, HYAL2, ETS2, RABL3, FAM171B, GP5, DEFB47,
SKA2, MARS2, STFA2L1, EPHA7, ATP1B3, HILPDA, POU5F2, CCND3, AKT1, BIN3,
TPPP3, ATG2A, ERGIC2, FMO5, PIGS, LYRM9, HSPA4, ACTN4, FAM171A2, GM15446,
SEC24D, SCOC, NGFRAP1, SLC23A2, MYH9, RP9, CDH3, FADS3, METTL10, POFUT2,
2310011J03RIK, A930017K11RIK, FLT4, NARS, TOPORSOS, DGCR14, CYSLTR2,
SH3GL1, KSR1, SRSF4, PVRL3, CCNG1, MRPL54, SEC62, GHITM, ARL5A, CDH5,
GM13277, GRAMD1A, MLH3, ATP2A2, LRRC8A, GM13306, SFN, KCTD5, DEFB14,
TRPD52L3, ABHD4, ELOVL5, ZFP804A, OLFR65, TPGS2, GOLPH3L, ZDHHC23,
OLFR1028, BIRC2,

TABLE 26-3
Module 3.12

SRP54A, SLC22A28, STAG2, USP9X, MATR3, XPO1, VPS13A, 7-SEP, GDI2, OLFM1,
DLD, VPS35, SNX13, RASA1, SLC22A30, HNRNPU, FNBP1L, BTAF1, SMIM10L1,
SACM1L, CLTC, ZFP703, DDX6, PPIP5K2, RANBP2, CASD1, ACMSD, GM14420,
STT3A, HNRNPA2B1, TMX3, IVNS1ABP, CUL3, GK, 2010315B03RIK, PNN, ZFP729A,
SRP54B, PAIP1, ZFP51, CAPZA2, ZFP729B, CSDE1, SMCHD1, CSE1L, ORC4, RAD21,
SCFD1, NUPR1, SLMAP, CIRH1A, C1GALT1C1, ATP6V1D, GM2411, CSTAD, ROCK1,
GM715, PLN, PCDHB7, ATP13A3, CALCRL, HMGCS1, PLPPR2, LCAT, MAFF, PSPC1,
HS6ST1, ATAD1, OGT, DNAJC13, EIF4A2, AFTPH, ATRX, CUL2, TBC1D32, TARDBP,
FUT8, UAP1L1, TTC37, STRAP, FBXO11, RPE, NAA15, 1500011B03RIK, GCA, JMJD4,
GDPD3,

TABLE 26-4
Module 3.13

GM2004, ZFP960, GM14399, GM14325, GM14308, ZFP931, GM14431, GM14305,
GM14326, GM14322, ZFP638, FAM208A, GM14295, GM14393, GM14391, ZFP433,
ETOHI1, ZFP97, RAB35, ZBTB41, PDS5B, FBRS, ZFP933, ZFP873, ZFP760, DOPEY2,
NDUFA6, CAV2, ZNHIT2, ATP11C, FPGT, TRAPPC2, 9830147E19RIK, UBA3,
GM14288, ZFP871, CLK4, POM121, PDCD7, TM9SF2, ZFP958, NDUFS1, PRR14, SDE2,
FZD7, 2610044O15RIK8,

TABLE 26-5
Module 3.14

DDX3X, DDX5, NCKAP1, USP47, IL13RA1, HSPA9, YES1, IREB2, HAX1, TM9SF3,
USP24, IPO7, TJP1, PPP1CB, GLS, SUPT16, BORCS7, STRN3, ABCD3, LIFR, CAND1,
PROS1, HNRNPM, IWS1, KITL, PANK3, LACE1, EPC2, ERICH1, MCUR1, 6-MAR,
A230046K03RIK, COL4A3BP, FXR1, RPP25L, B3GLCT, ATL3, LARP7, MAT2A,
EXOC5, CYP24A1, PDHA1, ZBTB44, SON, PHPT1, RAB1A, GIMAP4, ANKRD28,
CTNNA1, GNAI3, SPRR2B, TAPT1, ENOX2, HACD2, PREX2, KRTAP28-13, CCDC117,
LCMT1, TOP2B, ADAMTS5, EML5, TUBA4A, CD200, 1700024G13RIK, ADGRF5,
MDM4, EMC9, RWDD4A, NBEA, COG3, GTF3C3, SASS6, KLHL24, HOXA5, TM4SF1,
PDK3,

TABLE 26-6
Module 3.15

ETAA1, PPP1R9B, FAM133B, SARNP, PSME2B, RBM46, ZCRB1,
MIA2, NMI, RNF168, SFT2D2, AU019823, PLA2G16, NKAP, SLC5A1,
IFITM1, 2410016O06RIK, CR1L, HIGD1A, MOSPD2, MBLAC1

TABLE 26-7
Module 3.17

HSPD1, IFT27, UQCC3, BLOC1S3, HOXC10, SPCS2, RGS2, N6AMT1, RHOJ, ANTXR1,
ABCE1, HTRA4, NMB, AGFG2, ENSA, RPL31, FAM20A, NOP14, 2810474O19RIK,
DNTTIP1, TRIL, TFRC, GNB4, IRAK1BP1, ST13, IZUMO4, YPEL3, ADA, TBC1D7,
SLC25A30, UBQLN3, RGCC, MXRA8, RSPO3, GLTSCR2, PCDHB17, PRKAB2,
CAPRIN1, TRMT112, TMEM140, GTSF1L, TRP53RKA, GM590, SLC30A6, CLDN11

TABLE 26-8
Module 4.18

CSF2RB2, CSF2RB, IKBKE, CTSZ, ITGAL, RNF149, IRG1, NLRP3, SLC15A3, HCLS1,
F10, NCF4, LPCAT2, CD300LB, PRKCD, CLEC4D, IRAK3, CYP4F16, TLR2, PSD4,
MCEMP1, SLC2A6, TRAF1, IRF5, IL1RN, TMEM173, NFAM1, MEFV, LPXN, PTPN1,
ACP2, CSF3R, SLC5A3, TGM2, CTNNAL1, CTSA, IL17RA, SH3BP2, PIK3R5,
B4GALT5, PSTPIP2, SLFN2, TANK, FCGR2B, GRB2, NCF2, SLC38A1, ZMYND15,
BATF, TNIP3, CCL2, PID1, NEBL, LAT2, NADK, SERPINA1B, TREM3, PLEKHO2,
TBC1D9, PTPRJ, DENND4B, GPR84, NFKB1, DRAM1, ATP1A3, NRP2, LRRC25,
HMOX1, CYP4F18, NCAM1, MAPKAPK3, BCL6B, GBP2, SAA3, CD302, SH2B2,
STK17B, CCDC50, SH3PXD2B, NOS2, RAVER2, SNX18, DCAF6, ADCY7, PPP4C,
ORAI2, SMO, NLRC4, CH25H, BCL3, BTG1, PTP4A3, H2-M2, PLXND1, RNASE2B,
MMP9, BTG2, CXCL1, ADAM12, NCF1, RELB, BACH1, SLAMF8, CDC42EP2,
BCKDHB, PTGIR, 1700025G04RIK, CALML4, NUFIP1, OLFR267, SPATA13,
DCUN1D2, RGS19, CCDC88C, RASA4, ADAP1, NFKB2, IL10RB, VSIR, MOB2, NOD2,
D930015E06RIK, PLAGL2, GPR137B, MICALL2, PELI2, ATP6V1B2, GPR132,
TMEM176A, ST3GAL4, KLKB1, RRAD, LDLRAD3, LSMEM1, RUNX3, HIST2H2AB,
TM4SF19, IL1RL2, RNF26, ZFP799, PANX1, SVOPL, GPR62,

TABLE 26-9
Module 4.19

LILR4B, PIRB, CD74, GM14548, FGR, GM15448, PIK3AP1, MYO1G, PTPN6, LILRB4A,
TNFRSF1B, LCP1, ITGAX, NCKAP1L, FPR1, PIRA6, ITGB2, TYROBP, MS4A6C,
TREML4, IGSF6, CLEC4E, FCER1G, H2-EB1, PLEK, PILRA, LILRA6, CYBB, HCK,
CLEC4A1, RAC2, CD300E, CTSS, FYB, BCL2A1D, SLC11A1, DOCK2, GPR31B, FPR2,
SPI1, CLEC4N, MS4A6D, MPEG1, BCL2A1C, GBP4, CTSC, APOBEC1, PTK2B, CYTH4,
PTAFR, PTPRC, SMPDL3B, ADGRE4, CD300A, PIK3CG, CD68, IL1B, WAS, TLR13,
PLCG2, THEMIS2, ARHGAP9, NR4A1, ARHGDIB, GM11709, IKZF1, CD300LD, EVL,
POT1B, NAIP6, IL10RA, TNFAIP3, SYK, H2-K1, SRGAP1, CSF1R, MMP12, REL,
CX3CR1, NEURL3, CLEC4A3, H2-AB1, CD244, LYZ1, ALOX5AP, C1QA, PTPRE,
MS4A6B, ARHGEF18, PRKCB, CD274, ADRBK2, CD52, GPR65, GM21188, CD44,
SERPINA3G, IFI204, TLR1, CSF2RA, KLHL32, AI607873, APBB1IP, BTK, ARHGAP30,
ATP8B4, WT1, BCL6, LAIR1, FCGR4, CD53, GM5150, FAM49A, MS4A7, PPFIBP2,
STAP1, H2-DMB1, CCL8, CCL6, DOK3, GM6377, LILRA5, IRF8, C3AR1, IFNAR2,
RASSF4, PYGL, CCL3, STK10, GSAP, SLC16A10, SORL1, ANO1, LACC1, IFI27L2A,
B4GALNT1, ARL5C, B2M, PSMB8, PLAT, SASH3, PTGS2, GLIPR1, ALPK1, H2-AA,
CXCL16, BST2, TNF, CCL4, TNFAIP8L2, H2-Q7, MARCKS, TICAM2, RGS1, GBP3,
IRS1, HSPA12A, PSMB10, SCIMP, SNX10, GPR141, CD86, ADORA2B, ACE,
DENND1C, PILRB1, HEXB, MTCL1, CCR5, IFIH1, UBE2F, CKLF, ZBP1, C1QC, H2-T22,
CXCL10, PLAC8, FABP4, ICAM1, GBP8, ZFP185, LGR4, GBP2B, GBP10, SYDE2,
RPS6KA1, KCNAB2, CORO1B, KRT7, RNF213, CYTIP, USP13, PLXDC2, TRPS1,
FAM49B, MME, VMP1, CABLES1, ACER3, SHTN1, KREMEN1, TAP1, SAP30,
SEMA3E, REXO2, POSTN, TRABD2B, IL12RB2, GALC, NXN, PLEKHH2, MS4A4A,
CEACAM1, HAVCR2, H2-M3, CD4, TGFBR1, SLC15A4, MNDA, CCL9, IIGP1, ICOSL,
AIM1, HCST, RIPK2, H2-DMB2, DTX3L, TNFSF15, DAAM2, ABI3, PIP4K2A,
MTAP7D3, ABLIM3, ABCC9, EAR1, ZC3H12D, C3, RAB3B, SORT1, MEOX1, NCK2,
PARVG, SAMHD1, GM11710, PPARG, ABRACL, BCL2A1B, IFITM6, ZFP438, IFI202B,
IL18BP, DONSON, SMAD9, NAIP5, SERPINA3F, RIN2, TMEM219, GPAM, WSB2,
MYL7, GALNT7, CLIC5, HPGDS, NELL2, MAN1A2, SAMSN1, F2RL3, FCGR1,
ABCB1B, CD69, FCRL5, GNGT2, PARP14, KIT, CNIH4, SELE, S1PR5, PALMD, FAR2,
ADGRE1, SERPINI1, CHRNB1, SPINT1, AP3S1, KRT80, LTB, SLC9A9, TIPIN, CD72,
IL2RB, GLIPR2, LYST, PARP8, CKS2, NPR1, GM6498, TMEM204, ADAM8, CREB3L2,
SLC39A10, SCD2, TLR12, TRAF3IP3, PNO1, TRIM30D, CEACAM2, EMB, SPARCL1,
H2-DMA, SAMD12, CIITA, TMA16, ALDH1A2, HIST1H3H, EFCAB7, WFDC16, NOCT,
SULF1, SMS, SNX24, TSPAN5, TLR4, H2-Q4, KRAS, SASH1, ENPP4, CORO1A,
VCAM1, FMNL1, CYBA, SLA, PIK3CD, NFKBIZ, LRMP, AIF1, AF251705, CD37,
5031414D18RIK, PRCP, RHOG, LYL1, PILRB2, DCTD, TIFAB, NAIP2, A630001G21RIK,
SKAP2, MED12L, ZAK, POU2F2, ENO3, SYT9, SOAT1, ME2, GSDMD, ZFP521, UTRN,
PKP4, ARHGEF12, FAM105A, CXCL2, RAP1B, FUT4, B4GALT6, PARVA, MRPL52,
PEG3, FAM26F, PNP, SP140, ACADSB, CYP4F37, APOL7C, CD200R4, RILPL2, PODXL,
MICAL3, SIGLECE, ZEB2, NUP210, MAST4, PTPN3, FCRL1, GNA15, KLRB1C, NPM3,
JAM3, EYA1, ADRB2, GYG, EIF1A, JUNB, CTSE, LRPAP1, TMTC1, LOC100041057,
TBPL1, KANKI, CSF1, COL18A1, HHEX, VRK2, KLRA2, TLR9, BANK1, EOMES,
PIGU, TNFAIP8L1, KLK1B24, CEBPA, RBMS3, SIGLEC1, 6030408B16RIK, TOB1, GLA,
DDX43, 2010111I01RIK, TAPBPL, SERPINA3I, UBXN7, SIAH2, FAP, FBLL1, EFR3B,
OLFR348, CD40, VAV3, IRGM2, PSMD10, KCNK5, ALG14, CHRNA7, ABHD6,

TABLE 26-10
Module 4.2

CTSB, SELENBP2, GUSB, PLTP, ABCG1, TMEM182, ADRBK1, PSAP, CD200R1,
SLC43A2, FAM64A, PREX1, ADAM19, FAM13A, MCM3, HIST1H2AO, CENPN,
ALDH6A1, RORA, HOXC8, FOXS1, SELENBP1, SLC18B1, MFSD1, GEN1, ARHGAP32,
GNPTAB, FCHSD2, NDC1, LMO7, PARD6G, M6PR

TABLE 26-11
Module 4.21

GM20746, FTL1, IFITM3, ATOX1, TMEM14C, RPS11, TALDO1, SEPW1, TRAPPC4,
PSME2, GBA, NINJ1, HSDL2, RPL28, COMMD4, LXN, POMP, PPP1R11, GSTO1,
RAB8A, TXN1, LSM4, PSMB2, FBXL7, TUFM, KRTCAP2, IFIT3, HSCB, RPL14,
IFITM2, UBE2A, PABPC1, TRAPPC1, SPINK8, HSD17B1, GDNF, ATP2B4, WDR83OS,
NDUFS8, GLMP, CCDC80, PHLDA1, JUN, AMD2, ARL11, COA7, BBS12, PARL,
LAMTOR2, LSM12, PPIB, EIF3H, PSMG4, SNAP29, PSMD8, SREK1, MAP7, TSTD3,
TMEM86A, CEBPZOS, SEC61B, CALM3

TABLE 26-12
Module 4.22

HIST1H2AN, H2AFX, NT5DC2, TCF19, CCNE1, SLC26A7, ERBB4,
FAM102B, RPS6KA2, TTYH3, TFDP1, HIST1H2AF, NID2, TSLP,
ZFP72, 2610318N02RIK, ENO1B, FHL5, UBE2QL1, THSD1, GPR176,
ADCY4, SIM1, PCDHB12, ATP13A2, CTSD, PTTG1, OLFR1346,

TABLE 26-13
Module 4.23

OAS1A, SLFN8, LY9, HEXA, OAS2, LPL, IRF7, SLAMF7, OAS1G, KLK1B4, ADCY1,
IKZF3, FAM213B, TRPV2, JCHAIN, RTP4, GPR18, SELPLG, KLRA10, GAPT, USP18,
SLFN9, V1RA8, HEBP1, KLRA9, OASL2, PYCARD, KLK1B9, FAM96A, ITPRIPL1,
CYSLTR1, CAMK1D, CD5L, NME1, TBXAS1, ADRB1, SLC12A2, DDX58, GJB2,
PINX1, P2RY14, UBE2D1, GINS1, CHST7, STARD3NL, EXO5, OLFR460, SLC40A1,
TIMP3, DHX58, DST, ATP2A3, KLRA8, TTC33, S1PR4, CCDC86, TAGAP, GZMK,
STAT2, CYB5D1, CCL24, RORC, CMPK2, IFIT3B, FRMD6, C4B,

TABLE 26-14
Module 4.24

GM21119, ITGAM, 4930467E23RIK, GM7697, HP, CHIL1, GM15319, AB124611, CFB,
NTN4, N4BP1, EHD1, FERMT3, EMILIN2, LAMA4, SLPI, SLC7A11, CMKLR1, TREM1,
PTPRD, IFNAR1, TRPC1, CST7, XLR3B, HSPA1B, ACP5, SUSD2, GPX1, IL10, MYD88,
IL21R, GABARAPL1, MC4R, ELMO1, RND1, PKP2, PIK3R6, ZFP57, MADCAM1,
GUCY1B3, BST1, TNFRSF18, PLET1, MAPK1IP1, IP6K3, ABCC5, METTL7A1,
GM9495, RGS16, CLEC1B, SAT2, STEAP2, ZFP946, TUBB2B, FZD8, GDF5, PIK3R3,
GM614, INSIG1, MAL2, HMGCS2, PTPN7, MAN1A, MZB1, PPP2R3D, KCNT2, NARF,
PGRMC1, TRPM3, RAB8B, TMEM206, 6330416G13RIK, RALGDS, PRDX5, SLC39A4,
XLR3C, MSRB1, RALB, CYP2D22, MFSD7A, DUSP2, ARHGEF28, HNRNPAB,
ABHD12, PPRC1, BBS2, HIST1H4D, PECAM1, WLS, SLC51B, NHLRC1, PDCD1,

TABLE 26-15
Module 4.25

PHF11B, MS4A4C, XAF1, PYHIN1, PHF11A, IL18, GM8369, WDFY4, PSMB9, GVIN1,
NAAA, GM5431, TGTP2, GBP7, PHF11D, MAP4K1, PLBD1, KLRA17, RNF130, SP100,
CCL5, GM12185, COA3, SFT2D1, DDX60, BC094916, ISG15, NPY1R, ARL8B,
RNF113A2, UBD, CTSH, IRF4, RNASET2A, GRAP2, AI467606, IRGM1, PPT1, USP54,
ATF7IP2, TESPA1, MGL2, OLFR380,

TABLE 26-16
Module 4.26

CFHR2, TUBA1A, SH3BGRL3, CFH, SH3BP1, MAN2B1, TGFB1, TNIP1, TRP53INP1,
ARRB2, ACTG1, VASP, FES, UNC93B1, FXYD5, RASA3, CLOCK, PFN1, SLC8B1,
NFKBIE, PDLIM1, ABCD1, IER2, ARPC4, HTR1B, TUBA1B, PLAUR, TNFRSF1A,
ADAMTS16, APLN, PPP1R18, ETV6, UCHL1, AUNIP, P2RX1, SRGN, LTC4S,
TMEM176B, LSM2, ARHGAP25, CAMKK2, AMY1, ACTB, PDGFB, GBP9, SLC39A1,
SLC25A24, ACSS3, GM7221, APOLD1, TMEM123, VCL, LRRC8B, COLEC12,
TMEM192, OLFR1357, EXOC3L, GM4788, TAOK3, CTDNEP1, ADAMTS7, CDK2,
PPP2R3A, MTHFD2, SSSCA1, MEX3A, NEURL2, ADPRH, FAM46B, SDHAF1, MCTP2,
EPDR1,

TABLE 26-17
Module 4.27

PARD3B, MAGI2, SNCAIP, MYOM2, ROBO2, CD80, CFP, CD84, CORO2B, DACH1,
NTNG2, SCHIP1, SPIC, CD2AP, MSR1, NPHS1, TK1, CD93, GPR35, ST3GAL3, ABAT,
DDN, MYO5A, PTH1R, SEMA5A, COLGALT2, PRR5, GDA, PLIN2, TCF21, MAPT,
VEGFA, ASB15, ITGB5, E2F2, PTPRO, CD59A, FABP5, RHPN1, GGT5, PCSK5, H2AFZ,
ZNRF3, FLRT1, SGIP1, KLHL6, WIPF3, EPN3, FA2H, RP2, CLIC3, TMEM150C,
ANGPTL2, IL1A, PRR15, PDK2, FLT1, NPR3, COBLL1, NSF, BAMBI, NRP1, NECAB3,
FAM69A, PCNA, P2RY12, PCBD1, SHISA3, PTPN13, RNPEP, SLC38A4, DLL4, VSIG2,
DOCK11, MLKL, HOXB4, TSPAN15, LGALS4, MTSS1, EZH2, PTPRU, KCNJ5,
CD300LF, CEPT1, AK1, VDR, CHCHD10, SLC2A12, CENPH, RCAN1, TFEC, AU021092,
EDNRB, RAB10, AMPH, ARHGEF15, HOXC4, RAB29, SLC2A4, CD59B, PTCH1,
ALAD, SLC37A2, ATP5G1, CCNB1IP1, PLPPR4, SEMA6A, ATP6V0E, CLDN5, DDHD1,
ARHGAP24, LIN9, DDIT4L, TRAM1, RRAGA, DTNA, PITPNC1, EFNB1, MYO5B,
FXYD4, EPSTI1, TOP1MT, PLXNB1, TNFRSF11A, MICU1, SLC22A17, VANGL1,
INSRR, RASL12, MPP3, CRB2, SNRK, PKNOX2, RERE, ELL2, ERBB2, EGFL6,
COL3A1, DCXR, SERPINB8, EFNA1, TSPAN3, HIPK2, CISD1, SLC6A8, GDPD5,
HIST2H3B, USHBP1, OLFR1342,

TABLE 26-18
Module 4.28

HPSE, IL2RG, NR1H3, UBASH3B, ARHGAP4, SIRPA, CD55, P2RY13, MILR1, SLAMF6,
SPN, RAP1GAP2, CXCL9, SEMA4D, TSPAN32, NRROS, FAM65B, STOM, NFKBID,
ITGA8, ZFP697, MALT1, B430306N03RIK, MCOLN2, TNFSF10, HRH2, PLCL2, GDPD1,
ERC2, SLC7A8, CCL7, ACSL5, UPP1, D330045A20RIK, CCL12, SYTL5, TMC3, AREG,
TMEM106A, IL27RA, FOXN2, CYFIP2, COL15A1, SPON2, PRKAA2, EREG, CYP20A1,
DSE, SOD2, SOX4, RASGRP4, STMN2, BAZ1A, BLVRA, BMP15, NDN, ARXES2,
RERGL, PDYN, PIK3C2B, PPM1F, MAP3K7CL, GIMAP5, CD55B, CTSL, HVCN1,
TNFRSF14, ZC3HAV1, RAPH1, GM2A, IRF1, ELF4, FRMD4B, KLRB1B, EPS8, PGM5,
MFSD2B, HIST1H2BK, ADORA2A, BNIP3, PRRT4, EPHB1, RBM24,

TABLE 26-19
Module 4.29

LST1, CASP1, CD48, CLEC12A, SIRPB1A, ABCG3, CD180, CD83, CLEC4A2, WFDC17,
FAM111A, CCR2, CCDC88A, GMFG, AIM2, PLXNC1, CLEC4B1, EAR10, CASP4,
CLEC2I, TLR8, SP110, TRIM30A, EVI2A, RNASE6, PCDH17, I830077J02RIK, HGF,
ADCY6, ID2, A630033H20RIK, CD22, SNX5, NUP37, HFE, CDH1, BC028528,
ARHGAP15, SLC46A3, SCG5, MIS18A, GM9733, DNASE1L3, C130026I21RIK,
SLAMF9, MTHFSL, RASGEF1B, LGALSL, TPCN1, CLEC2D, TMED5, MGAT2, PPIH,
TMSB4X, TTLL4, FCMR, GAS7, SH2D1B1, NAT14,

TABLE 26-20
Module 4.3

PLCE1, THSD7A, ASTN2, CACNA2D2, ARHGAP28, CLIC1, AEBP1, CCDC109B,
FCGR3, SH3BGRL2, CD47, OPTN, ATP8A1, MPP5, MSN, EPB41L5, TBC1D8, LRRC49,
SPATS2L, STK26, FRAS1, PLEKHA5, GOLIM4, ARVCF, LCTL, VTCN1, SNX31,
S100A4, TM6SF1, FLNB, MCM6, C5AR1, ZRSR1, RAB6B, ASF1B, ST6GALNAC3,
DMPK, PTPN22, FAM184A, DUSP15, DPEP2, VWA8, MYO1D, GRIN3A, PDE1B,
TSHZ2, PPP1R14A, TUBB5, MBNL3, IQGAP1, AIDA, TET1, ABCC3, SNTB1, AKR1B7,
LY75, LGALS3, GFPT2, APOE, PARP4, BLM, KCNMB1, TXNRD1, RASGRF2, IL1R1,
MAN2A1, RAPGEF4, ADAP2, NGB, SLC7A6, STARD5, AP1S2, FXYD6, PPIL6, SDPR,
CACNA1G, FAM129A, MRC1, ZFP462, R3HDML, CSK, KLF12, MCC, RPL7L1, NPC2,
SGPL1, ZMAT4, FGD3, GRIP1, CDC14A, DYNLL2, FAM222A, 11-SEP, UQCC1, NRIP2,
TUBB6, RASSF2, ACOT9, DYNC1I1, SLC31A2, APRT, LY6I, CYTH3, SYT15, DIAPH1,
RGS6, LEPR, GCNT2, HEY2, NIPSNAP3B, PNPLA7, SLC9A7, MRC2, NBAS, GLRX,
PLAU,

TABLE 26-21
Module 4.31

TDRD5, NARS2, PAK1, NPHS2, SIK1, NEK1, ARHGAP12, GM15453, GADD45A,
DOCK4, BIRC3, RBM3, OLFR558, UPRT, TTC39B, PPP1R14C, NPAS2, NAT2, PSMA6,
EID3, RAPGEF5, TAAR2, HSPA1A, TMEM14A, DENND4A, PCDHB11, MICU2,
E430025E21RIK, MGAT5, INPP5D, SPATS2, NLK, SPATA24, ANTXR2, FAM3C, PFN3,
TDPOZ3,

TABLE 26-22
Module 4.32

SLC28A2, A, ITGA3, ITPR3, CSPRS, GM7609, UNC13B, COL4A3, ECSCR, LRRC55,
FGFR2, MS4A4B, ZFP385B, EFNB2, C130050O18RIK, COL4A4, ELMO2, AIF1L,
C1QTNF1, GM14085, SDC4, ARHGEF26, OLFR111, LPAR4, DSP, ICOS, LUM, GPC1,
AKR1C18, TSPAN2, OGDH, ST6GAL1, SLC41A2, DLG5, TXNIP, PRKD1, PLPP1, EZR,
ITK, ERLIN2,

TABLE 26-23
Module 4.33

ADGRL3, ZFP273, ZBTB20, CLEC5A, TRPC4, MAPKAPK2, ABI3BP,
DGKB, STXBP4, GSR, LDHA, PDE5A, ZFP85, KPNA2, LGMN,
KLHDC1, EEF1E1, UPK1B, 5031439G07RIK, UNC5C, MCM4,
GRK5, GOLT1B, SLC25A27,

TABLE 26-24
Module 4.34

MGLL, AZIN1, SAT1, TPD52, ITGA4, D630045J12RIK, LYN, CAPZA1, RAP1A,
NFE2L2, CTDSPL, HOXA9, EZH1, PHF1, SPIRE1, NABP1, TMEM108, SHROOM2,
FOXC2, HOXA6, KCTD12B, RASL11B, SH3BGRL, CD34, ARL4D, CYP2R1, MOB1A,
LTBP4, ZBTB7C, METTL20, PRELP, 4921507P07RIK, ACOT1, SESN1, SMIM15,
CCRL2, FZD4, POLA1, PTPN2, PDGFRB, SLC1A4, RBMS2, PALLD, KCNF1, ZFP651,
MORC3, SLC25A35, TPP2, SH2D4B, MRGPRA1, TMX1, IDS, TMEM2,

TABLE 26-25
Module 4.36

RCAN2, RBL1, RAB3A, UFSP1, RTN4RL1, FAM214A, GK5, LIMS2,
ZDHHC14, TFDP2, PDGFC, CCDC3, KCNMA1, IGTP, GABRB1,
GIMAP7, TAB1, IGF2R, PTEN, ANKS1B, GDPD2, ITLNB, FECH,
HIST1H2BH, CEP126,

TABLE 26-26
Module 4.38

ACTR3, RSPH9, DUSP16, SERP1, SPPL2A, D15ERTD621E, 4930538K18RIK, GALNT1,
CALU, SP4, RHOA, BZW1, SDCBP, SPRED1, EGR3, FBXL5, SPA17, OIT1, IL1F9,
OLFR1393, EIF2AK2, KLF15, TMEM95, OLFR1490, PGK1, NXPE3, API5, MORF4L2,
RAP2C, AGO4

TABLE 26-27
Module 4.39

CD3E, CD3D, CD3G, NLRC5, CD5, CD8B1, ATG4A, MS4A4D,
LAT, ALG8, SDF2L1, RASAL3, STAT1, IL7R, 2210408I21RIK,
JMY, GZMA, SELL, TAS2R108, IRF9, STK4, CD7, TMEM229B,
HAPLN1, 1700092M07RIK,

TABLE 26-28
Module 4.41

ARNT2, EPHX1, PLPP3, GALNT10, NT5E, ILDR2, NPNT,
TNS2, KIRREL, AJUBA, KCTD12, ISYNA1, GM527, HTRA1,
PTPN14, ARHGEF17, GATA3, ACAA2, MYLIP, EXOC3L2,
PXDC1, CYSTM1, GAREM, PDPN, TBX3, CDKN1C, HS3ST3A1,
SYDE1, SNX33,

TABLE 26-29
Module 4.44

NR2F2, RAP1GDS1, CPED1, MBIP, TMEFF2, FKBP7, MNAT1,
QPCT, UPF3B, SMIM4, CIR1, FXYD1, TMEM136, GJB4,
AGA, ENPP5, IGFBP4, ZFP955B, 4930523C07RIK,
TRIQK, GIN1, ELFN1,

TABLE 26-30
Module 5.53

SLC5A12, ACSM1, ACSM2, ARAP2, HSD3B2, SLC16A12,
GM16432, OXCT1, GATM, UMOD, D630023F18RIK, PRDX3,
SORD, SLC47A1, SLC26A1, GM7068, GSTZ1, SELT,
FMO1, SLC22A12, PRODH, LTBP2, PPP1R16B, MPZL2,

TABLE 26-31
Module 5.54

LRP2, UGT3A2, PAH, SLC34A1, SLC7A9, CUBN, CYP2J5, MIOX, KEG1, SLC6A20B,
TMEM27, SLC4A4, CAR4, HGD, ASPDH, UGT3A1, SLC10A5, SLC27A2, BC089597,
RDH16, CYP2D26, APOB, AADAC, ALDOB, UGT2B38, UPB1, DNAJC12, SLC22A1,
HABP2, PM20D1, CYP2J11, AK4, FXYD2, SLC22A6, BC021785, SLC13A1, PBLD1,
KCNJ16, SLC17A1, PROM1, GALNT11, SLC1A1, TMEM8, FBP1, PAPSS2, FAM151A,
CLRN3, PTER, HNF4A, PHF19, SLC3A1, GM11128, DMGDH, ACSS1, ALDH4A1,
CAR12, SLC22A5, G6PC, PCK1, GGT1, FABP7, CHRNA4, TMEM116, HYKK, SLC2A2,
SLC22A22, UGT2B5, SUCLG2, ACY3, ACAD11, CYP4A32, HAAO, SUCNR1, ACE2,
SLC5A8, SLC23A1, KMO, SLC25A10, CIDEB, ABCA13, SLC22A2, CRYZ, CREB5,
IGSF11, ENPP3, THEM7, LPAR3, SLC12A3, GPM6A, PAQR9, RBP1, MEP1A, LSM7,
METTL7B, OLFR64, RANBP3L, KAP, LMBRD2, RAP2A, LRAT, CAV1, SLC22A19,
SLC35E3, GPX3, SPP2, SLC9A3, ACAT1, SLC25A21, ENPP6, SLC17A3, PBLD2,
HSD3B3, PCX, VIL1, ND6, GCDH, ALDH9A1, SLC44A4, SOD3, TIMD2, GCNT1,
RETSAT, WDR74, AU041133, CAT, SLC25A15, ACY1, ANKRD44, TRDMT1, IDH1,
UGT2B37,

TABLE 26-32
Module 5.55

SLC22A29, GM8765, HAO2, CLDN10, TINAG, MDH1, ADHFE1, ASPA, GC, SLC16A4,
PDZK1, PECR, SULT1C2, SLC16A7, SLC22A27, WFDC15B, SLC19A3, TPMT, CAR2,
PHYH, ESRRG, SUGCT, 1700011H14RIK, LYPLAL1, SLC25A36, SLC8A1, HADH,
FAHD1, BDH2, ARSK, GALM, MTHFD1, KCNJ1, SLC22A18, TMEM139, GM906,
CML1, BSND, SMIM22, TTR, MYO9A, GYS2, DAB2, CRYGS, SDCBP2, NT5C3, PGF,
PDE7B, PRLR, ADTRP, HIBADH, SMIM24, SLC37A4, ANGPT1, PLS1, PLEKHA6,
SUCLG1, GRTP1, YAE1D1, PHKB, SEMA6D, PRPS2,

TABLE 26-33
Module 5.56

NOX4, CES1D, AASS, F13B, LRRC19, FUT9, PIK3C2G, CTH,
A1CF, FMO2, CES1E, UGT1A2, KYNU, ALDH8A1, GLYAT, CLYBL,
RNF128, AQP11, ABCC2, IGFBP1, OLFR920, DPYD, MGAM, CLDN2,
RHOD, CES1F, AGXT2, TMIGD1, PMP22, HMCN1, 4931406C07RIK,
PHYHIPL, PRODH2, LACTB2, SLC16A9, PXMP4, MOGAT1,
B3GALNT1, CES1G,

TABLE 26-34
Module 5.57

KCNJ15, FOLR1, SLC6A19, XYLB, PIPOX, ALDH1L1, PRSS8,
NDRG1, TMEM174, CDHR5, TMEM106B, DAO, GPD1, P2RY2,
DGAT2, NR1H4, CYP4B1, GLDC, ABHD3, TMEM30B, AQP1,
HSD3B4, WNK4, SLC6A20A, CML5, CRYL1, SLC16A14, TCN2,
ACADM, MSRA, GRB7, TKFC, AS3MT, BC025446, HSD3B6,
MGST3, SLC6A13,

TABLE 26-35
Module 5.58

SLC12A1, CDKL1, KNG2, TMEM72, ATP6V0A4, PKHD1, THBS2,
ARRDC3, CALB1, CDH16, EFHD1, PPARGC1A, PSD3, PPP1R1A,
WISP1, SLC26A4, CLCNKB, ATP6V1B1, ATP1B1, CLDN16, SCNN1G,
SGK2, CASR, SOSTDC1, CAPNS2, CAR15, LDHB, ATP6V0D2,
HSD11B2, ALCAM, CXCR6, OXGR1, ANXA9, SCNN1A, KNG1,
CROT, KLHL3, BRCA2, RHCG, TMED6, FABP3, NADK2, WDR72,
MYOM1,

TABLE 26-36
Module 5.59

SERPINE1, SLC34A3, CADM1, KL, TNC, CYP1B1, GALNT14, KLF6,
CRIP1, ANXA3, SLC5A2, BTNL9, LSP1, SLC18A1, LCN2, PRSS23,
PDK4, NRG1, AMICA1, CCL17, SGPP2, SLC7A1, XKR4, CLDN8,
GLOD5, PLOD2, TRF, ANXA1, SERPINB6B, ANGPTL4, C2,
OGFRL1, POLR2H, RAB7B, EXT1, TES, MPPED2, ECI3,
SRD5A1, SLC9A2, DCLK1, TATDN2, TMEM102, GM19668,

TABLE 26-37
Module 5.61

SLC22A8, WSB1, SLC13A3, ASS1, LRRK2, COX7A1, ATP1A2,
ADGRL2, SLC51A, 1810010H24RIK, GM5622, CHORDC1,
PDGFRL, ENPP2, TIGD5, TCF23, TDRKH, SRPX2, GATA6,
PLSCR1, EPB41L3, MED17,

TABLE 26-38
Module 5.63

ND3, ZFP825, DMXL1, HRSP12, UQCRC2, HDAC2, CAB39L,
FH1, ND4, PPP6C, RIF1, TLE3, ARRDC2, FAR1, UBP1,
NEURL1B, TMEM30A, FASTKD2, SYAP1, FUCA2, RBM25,
GGH, UBE3A, CUZD1, ING3, DECR1, BRAP, 10-SEP, SAR1B,

TABLE 26-39
Module 6.79

HIST1H2BL, TOP2A, KIF23, NUSAP1, KIF11, KIF15, PRC1, CASC5, CDK1, ASPM,
DEPDC1A, HMMR, HIST1H2BN, DLGAP5, CENPE, STIL, GAS2L3, MIS18BP1,
PLA2G7, CKAP2L, CEP55, CENPF, SGOL2A, KIF20B, HIST1H2BA, CENPW, NUF2,
NCAPG, KNTC1, BUB1B, RAD51, CKAP2, NEIL3, KIF2C, FIGNL1, E2F8, CDKN3,
OLR1, HIST1H2BF, GM15056, SMC2, ATAD2, SHCBP1, HIST1H2AK, MAD2L1,
HMGB2, GDF10, AOAH, PARK2, NCAPG2, ESCO2, ANPEP, HS3ST3B1, HELLS, OIP5,
F7, TTK, ARMCX4, WDHD1, CENPM, HIST1H2BJ, PLOD1, GSTA4, ACKR3, SLC35G1,
BRIP1, AKR1C19, ACHE, HIST1H2BB, RFC4, ARG2, NPY, MUC1, C330027C09RIK,
ZFP2, HNF1B, HIST1H2BM, CYP27A1, PLK4, CDCA2, DLGAP1, AURKB, BUB1,
INCENP, SMC4, RACGAP1, CLSPN, ARHGAP11A, CENPI, ZWILCH, NCAPD2, UBE2T,
NSL1, CIT, PPMIN, ZDHHC13, KIF18A, PCP4L1, SERPINH1, CHEK2, ADAM10, FGA,
GABRA4, 4930503L19RIK, MSH6, ABCD2,

TABLE 26-40
Module 6.8

TPX2, CCNB1, CCNB2, STMN1, MKI67, DIAPH3, ANLN, KIF22, MCM5, UBE2C,
RRM2, PLK1, DTL, PRR11, TACC3, SKA3, KNSTRN, CKS1B, PBK, NCAPH, GPNMB,
CLMN, CDCA8, ULBP1, CDC6, COTL1, NDC80, KIF20A, MCM2, MASTL, E2F7,
FOXM1, ECT2, 2810417H13RIK, EPCAM, CHEK1, MEST, SPC24, CDC20, KIF4, BIRC5,
CCR1, DDO, UHRF1, SFRP2, LIG1, TOPBP1, SGOL1, ADH1, GRM7, GSTT2, EFEMP1,
GMNN, BC051142, BMP7, AURKA, PLPPR5, CDKN2D, IQGAP3, TRPM6, OLFR524,
HINT2, FAM83D, FBX05, SSFA2, MS4A8A, NME3, NR3C2, FDPS, OLFML1, PARPBP,
DBF4, ADGRL4, GM7694, CYP39A1, PRIM2, 8430408G22RIK, CDKL5, CDC45, LRIT2,
CENPK, POLE, TRIP13, KIF18B, TNFAIP2, CCNE2, CENPP, SKA1, HIST1H3E, PLA2R1,
MED11, DGKG, A530016L24RIK, SPON1, TMEM213, KLK1B1, DUSP9, TNS3, DPEP3,
TTC8, TMEM45B, ANKRD46,

TABLE 26-41
Module 7.84

XLR4B, ADAMTSL5, XLR4A, GSN, TEAD2, MYO1B, CRISPLD2, LAMB2, MXD4,
CORO2A, FGFR1, TMEM26, APLNR, TSPAN7, ITGA7, ZFP646, P4HA2, USP53, RSAD2,
AMIGO2, GAB2, DTX3, GPC6, BMP6, NBL1, CDO1, PDE3A, PER1, XLR4C, CPE, PI16,
PTPRB, ZHX3, SPSB1, FOXO3, FRMD3, MAL, NKG7, SCN3A, ST6GALNAC2, MMP15,
HOXD10, ZFP618, KLRD1, USP2, FOXQ1, CSRP2, TSPAN1, TXNRD3, HIST1H4I,
ESYT2, GCLC, KANK4, CCND2, OCIAD2, RTN4IP1, FOLR2, HYPM, LAPTM4B,
PRELID2, METRNL, CD151, CUEDC1, AFF1, RRAS2, KLHDC8A, SORBS1, SFSWAP,
PAN2, CD226, IER5, CD96, CACNA2D1, NLRP1A, FKBP14, HECTD2, PINK1, CLDN7,
GNPDA1, AI317395, SLC19A2, SLFN1, BSPRY,

TABLE 26-42
Module 7.85

FN1, DNASE1, TGFB3, CYP2D11, ACTN1, VIM, CYP2D10, TAGLN, DAP, CLEC2H,
HPD, PVALB, CDK6, ACSM5, GPRC5A, MMP2, FBN1, CYP2D12, PTGFRN, GPX6,
DDC, AFM, OLFML2B, TIMP1, RALGAPA1, ADAMTS2, DHTKD1, SHISA4, COL12A1,
YBX3, CAR14, COL1A2, CYP2D9, TMEM260, CDH11, COL4A2, NAT8, RPA1,
NSMCE2, AKAP12, GDF6, FAT4, COL1A1, CYP2E1, FAM19A1, TMTC2, PIGR,
NOVA1, TNFSF13B, MMP10, GPR39, LAMA2, CYP2A4, RRM1, VMN1R27, NPBWR1,
SLC35F6, COL4A1, CYP2D34, SLC39A6, TPM2, PDZK1IP1, CRLF1, TPM1, MYL12B,
ACOT3, SLC6A4, TGFB1I1, PDGFA, SYT12, ALB, MUT, PTPRQ, F3, LBH, GM904,
P3H4, OLFR1511, TDPOZ1,

TABLE 26-43
Module 7.86

CLCA3A1, SLC6A6, MAP3K6, WEE1, HHIPL1, STIM1, AGAP1, SGTB, ITGB3,
CDKN1A, POLN, SEMA7A, TSC22D3, INF2, FKBP5, PIM3, TUSC5, SORBS3,
FAM208B, ARHGAP31, SIDT2, P2RY1, MLXIP, GLUL, CLCA3A2, LRRC16A, ELL,
DDIT4, ZBTB16, ELN, SUN2, NQO2, CDC42EP4, CLIP1, CEP170B, TMOD2, VPS11,
MARK4, ENTPD1, KLF13, ROM1, XDH, PDE2A, CHML, RNF169, RASD1, FAT1I,
B3GALT2, FLRT3, TAAR5, PBX1, ZXDB, PLPP2, FADS2, SPATA5L1, ZFYVE1,
MFSD2A, FAM210A, PLCD3, CSRNP1, A430105119RIK, SMAD6, LY6C2, MCF2L,
CHMP7, SDHAF3, ACKR2, SCD1, HIPK1, FAM189A2, OLFR1414, 1810062G17RIK,
VMN1R168

TABLE 26-44
Module 7.87

GM3591, GM3636, GM10406, GM5796, PER2, GM3383, GM2237, GM3317, GM2897,
GM3264, DBP, GM3558, TEF, CRY2, PER3, CIART, AVPR1A, GM2956, CPEB1,
GM3667, ARNTL, BHLHE41, LEO1, CYS1, GIMAP3, NREP, NR1D1, IL6RA, PI15, HLF,
TSPAN4, NR1D2, AR, VSTM4, GSTT1, ZIK1, GNAT1, GM3696, GM3500, RAB3D,
KCTD2, IFNGR1, FAM76A, TMEM71, HOXB5, CRISPLD1, OLFR878, MEIOB,

TABLE 26-45
Module 7.88

1700049E17RIK1, CD109, ST8SIA2, ASTN1, SORCS2, PHGDH, IGFBP2, FLNC,
LOC101056159, PTGIS, CRIM1, EPHA2, IGFBP6, LAMC2, 1700049E17RIK2, CLDN1,
PAX8, MAP1B, PLCB1, ARHGAP20, SERINC2, DES, TMEM171, FAM180A, TACSTD2,
ADAMTS8, CDH6, KRT18, 4930503E14RIK, FREM1, ITGA2, ECM1, MYOF, ANXA8,
KRT8, AKR1B8, CMTM8, APLP1, SRGAP3, FST, NES, EDIL3, GXYLT2, RTP3, CLDN4,
STEAP1, ADGRG2, APOL10B, SOX9, SBSN, SLC17A9, SNAPIN, MMP7, SYNC, KLK8,
P4HA1, CLDN3, GM22, TTLL1, E2F3, LY6G, ALDH1A1, LYPD6, CYP26B1, MIDI,
GLI1, CFTR, TIMP2, PRKCQ, SHROOM3, SERPINF1, GM8267, FBLN2, RGL1, CNN1,
CAPN2, SECTM1B, PLEKHG1, ZSCAN25, CDH2, CCBE1, PDXP, SLIT3, ICAM2,
WFDC2, PKDREJ, SLC24A3,

TABLE 26-46
Module 7.89

CD248, FIGF, PAMR1, AXL, LOXL1, ANGPTL7, VOPP1, CES2G, CEBPD, MICAL2,
S100A11, IL34, KLHL30, AMPD3, PTRH1, SLC7A5, TNFRSF12A, PCDH19, ADAM4,
SLC10A6, CES2E, GIMAP9, PCP4, AHNAK, DUSP6, B4GALT1, ZFAND2A, TFB1M,
DUSP1, TMEM200B, PTN, MS4A2, F8, PYGM, PLA1A, IL1R2, CMAH, SLC43A3,
KCNJ12, HMGN5, LDLRAD4, LRRK1, TUSC1, SEMA4B, LTF, ARHGEF25, NCS1,
NIPAL1, MARK1, GREM1, FIBIN, S100A7A, ARC, TMEM63C, PLCB4,

TABLE 26-47
Module 7.9

PRRG4, DARS, ZFP937, PEAR1, ADGRA2, IRS2, PMEPA1, KLHL29, AGAP3, TRIP6,
PRKCG, CXCL13, SAMD4, FSTL3, P2RX5, CARHSP1, CPT1C, 2200002D01RIK,
SORCS1, ZFP442, ITGA6, LDB1, ARHGAP18, ST8SIA4, PPP1R15A, CACNG7, MED13L,
RNF41, GPBP1, KALRN, TLE1, RBPMS, SMOC1, OLFR1396, SCARF2, PLXNA2,
CERCAM, FARP1, HSPB2, ST8SIA6, LIMD1, RASGRP3, BMP2, ZCCHC14, ERCC1,
BAIAP2, ZFP934, VAV2, NPL, SLC35E4, MXRA7, AOX1, LTBP3, SUCLA2, SFRP1,
GM10094, TGFB2, ZC3H7B, HRCT1, ITPRIP, FAM174B, PEA15A, EPHA6, LRRC75B,
FKRP, AGER, JADE2, INTS6, IFT43, EXOC6, ULK1, PTGS1, RMND5B, FEZ2,
D130040H23RIK, PDZD2, NTRK3, PNPLA6, CAMK2N1, FYN, MT1, ZDHHC1, TACC1,
HSPB1, IGFBP3, SLFN3, CERK, PNKD, GM5141, BSG, 5430416009RIK, CYP4F17,
PSMB11, DNM3, CCNJL, ETD, LONRF3, PLCL1, GM3604, DUSP18, NANOS3, 4-SEP,
DEFB48, HOXA11, GIPC3, CYB5R1, TPGS1, PLEKHG3, PHKG2, ITIH5, PSEN2, RAD18,
SMAD7, RCAN3, MKL2, GATA2, R3HDM4, MNT, ADARB1, PLEKHH3, PCED1B,
CCNYL1, CAR3, GTF2I, DNM1, HOXA10, MANBAL, RAB3IL1, GPR173, SLC37A1,
GNAO1, 2010109A12RIK, IFIT1BL1, LAYN,

TABLE 26-48
Module 7.91

SFXN3, QSOX1, F2R, ITGA5, KLF4, LRP1, NDST1, HBA-A2, VAT1, HMGA1-RS1,
ABCA9, FOSL2, MTHFD1L, HBB-BT, REEP4, PLEC, UCP2, ABCA7, ODF2, NEPN,
DOCK8, KBTBD7, CDH13, BMP3, PLP2, METTL4, MT2, CENPT, GPRC5B, COQ10B,
ZMAT1, PQLC3, CXX1B, LMNA, ERN1, GNS, SPTSSA, PDLIM7, 4932429P05RIK,
LRRC32, ADGRE5, PLCD1, SDC1, HIST1H2BE, AKR1E1, HBB-BS, PLCG1, SLC38A2,
D3ERTD751E, CD33, PFKFB3, P2RX4, LTBP1, LOX, SLC26A10, LYRM4, EIF6, GEM,
CARNMT1, P2RX7, LIPO1, HEG1, SLC20A1, RNF144B, KCNAB3, FUCA1, IKZF2,
GNAI1, THBD, SVIL, BPHL, LRRC3B, JDP2, PCDH12, THAP2, CALCA, DAPL1, ARF2,
GRASP, C7, RNF125, PLEKHG2, BMP1, KLF3, BAG3, IL4RA, OLFR609, DCLRE1B,
AAGAB, ITPRIPL2, ACYP1, PKN3, S100A3, GCSH, RPL36, NOS3, 4931406P16RIK,
CNN3, USP11, CPT1A, GALNT4, CCL28,

TABLE 26-49
Module 7.92

SMURF1, ANXA11, LAMA5, TEAD3, SLC25A28, QPCTL, NOTCH3,
PKD1, NCDN, MAF1, KIFC3, MIDN, ZFP930, CTGF, PLXNA1,
CSPG4, RERG, XCR1, FGD4, HIST1H2BG, CLEC9A, H6PD,
CD36, PRKCA, RGS5, GALNT18, CTDSP2, BTLA,
1110038F14RIK, SGK3,

TABLE 26-50
Module 7.93

FGFR3, DENND3, FAM117B, INPP5K, HYAL1, SULT1A1, FAM107A,
TNRC6A, ATG14, LY6C1, CDR2, FAS, CLEC1A, SGK1, LPIN1,
LAMA3, CAPN5, INSR, KLF2, BCKDHA, NAMPT, TESK2, LNX2,
9430020K01RIK, MERTK, SHROOM4, NOP58, DHH,,

TABLE 26-51
Module 7.94

RNF152, IFI203, PPP1R1C, IFIT1, IFI44, PYDC4, TIMP4,
TTLL7, TFPI, FBXO40, DZIP3, MYOCD, TNNT2, ZADH2,
SPRY2, NQO1, PYDC3, H2-T24, TEX35, TBC1D5, CHFR,
TAS2R143, PERP, TRIM34B, SLC23A3, NSG1, HERC6, OSTC,
H2AFB2, OLFR584, KCNJ10, SENP8, TRIM9, 4930444G20RIK,
SPHK1, RAB1B, A3GALT2, GALNT15, PABPC1L,

TABLE 26-52
Module 7.95

ANK, ELMOD1, SERPINE2, SETBP1, CP, DKK2, ISLR, PLVAP,
OSMR, LHFP, ANGPT2, CPNE7, SLCO3A1, NRXN3, DMD, ROR1,
PSMG2, CPNE4, STK38L, DYNC2LI1, AKIP1, CHST15, BMP4,
TSPYL1, TOX, ZSWIM7, PCDH7,

TABLE 26-53
Module 7.96

INAFM1, HSP90B1, FBXO32, ACER2, LMBR1L, BMPER, MAGT1, ZC3H6, HSPA5,
ASCC3, LRIF1, PIK3IP1, 1700109H08RIK, 1110017D15RIK, CORO6, THAP3, CALR,
FUBP1, PDIA4, CIRBP, SERPINB9, PPP1R3A, DNAJA1, SLC38A11, MSS51, HSPH1,
GPR157, ABCA6, MR1, UBE2N, CACYBP, OLFR1392, ZFP580, HNRNPA3, MSL3L2,
OCIAD1, CD46, SLCO1C1, 1810024B03RIK, OLFR78, 5730480H06RIK, PRDM1,
CYP26A1, INTS3, TBX6, PHYHD1, GRK4, DMTF1, ZFP687, UVSSA, PDIA3, MYRIP,
ZFP526, IFFO1, 1700034O15RIK, 1700047I17RIK2, FRAT2, RNASE12, 5730455P16RIK,
FAM209, OLFR38,

TABLE 26-54
Module 8.101

HPRT, GNG5, RPL22L1, SHFM1, TCEB1, HCAR2, FAM174A, SSB, UCHL3, TLR3,
ZUFSP, N6AMT2, ENY2, PSMA1, TMEM258, CFDP1, RDH14, ZFP821, GM13199,
1700020D05RIK, CNIH2, TMEM209, SRI, DEPDC7, CHAC2, 2-SEP, AMIGO3,

TABLE 26-55
Module 8.102

POLR2F, SRA1, TIMM17A, FIS1, DDIT3, PSMB6, MIF,
1810037I17RIK, 2310036O22RIK, ICT1, MRPL11,
NDUFB7, VPS25, ID3, CUTA, GSTP2, HSBP1, CD300LG,
PCDHB6, DPM2, ZCCHC3, SNCG, HMGN3, FBRSL1, PDE10A,
NDUFA11, RNF141, SIX2, DOLK, KCNE1L, S100A13,
IMP4, OLFR686, ACTL7A,

TABLE 26-56
Module 8.98

RPS27L, DYNLT1F, TCEB2, RPS27RT, ERH, PRDX4,
MAGOHB, SF3B6, PSMB1, PSMA2, SEC11C, SNRPF,
EID1, HACD4, ATG5, DYNLT1B, RPS25, TRIM44,
DCDC2A, NSMCE1, TAF1B, UQCC2, 1700013G24RIK,
TMEM256, TMEM29, RPS4X, CMC1, CHCHD1, GM14151,
RNF223, LRRC26,

TABLE 26-57
Module 8.99

3830403N18RIK, 9930111J21RIK2, TLR7, TBCA, SDHAF4,
FCF1, MRPS18C, RPF2, MED30, SAMD9L, PIN4, 1810022K09RIK,
RASSF8, CCDC58, SREK1IP1, LY6K, CBWD1, WBP5, LPAR6,
XLR, SHANK3, ARXES1, VRK1, H2-OB, 9930111J21RIK1,
ESF1, TIMM10, SNRPG, AKR1C13, DCUN1D5, APLP2,
2700060E02RIK,

TABLE 26-58
Module 9.104

CCDC90B, MINOS1, TOMM7, CETN3, BLOC1S4, LYRM2, NDUFA1,
CCDC66, POLR2K, SNX16, SWI5, JKAMP, ARL6IP1, CTLA2B,
DNAJC15, VTI1B, TMEM179B, TMEM126A, MAGOH, COX6B1,
COA6, GTF3C6, PDCD10, MRPS36, TMCO1, CHM, CMTM7,
EIF1B, CTLA2A, NOL7, MRPS28, ADO, CNPY2, SPACA4, MED31,
GPR33, GTPBP8, D230025D16RIK, DPH6, NUDT7, NDUFC1,
HIF1AN, CSNKA2IP, TIMM8B, APIP, SELK, POLE4, SUGT1,
SLAIN2, ATP6V1E1, MRPL17, EFCAB2, PCSK2, NUP35, COX11,

TABLE 26-59
Module 9.105

MED21, NDUFS4, ETFA, 0610037L13RIK, IFT20, SLIRP, UQCRB,
USMG5, UBE2V2, MBD1, PNPT1, NDUFA4, DNAJC19, TAS2R126,
HSPE1, COX7B, 2010107E04RIK, COX7A2, KLHDC3, HNRNPC,
CHCHD3, SCN7A, CYCS, ZFP518A, RPA3, CCNH, SNAPC5,
TRIM12C, NDUFC2, MCTS1, JRKL, L7RN6, SUMO1, DHFR,
PIGF, PKIA, MOB4, BAHD1, SNRNP70, LSM3, FUNDC2, CYTH1,
SNX6, GM4924, RCVRN, ZFP768, EPHA3, PON3, GRRP1, FGFBP1

TABLE 26-60
Module 9.106

GM2964, NDUFAF6, POLR2G, CHURC1, ZFP639, NDUFA2,
NDUFA13, NDUFB9, UQCR10, BC031181, TUSC3, ATP5K, GATC,
YARS2, PRORSD1, ALKBH7, OLIG1, ETFB, GM561, S100A1,
ANAPC13, IDH3G, FRK, OLFR1162, KCNG3, DKKLI, ESM1,
GM21699, PRMT6, UQCR11, SNRNP27, PLSCR2, ZNHIT6,

Table 27-1 to 27-48: MEGENA gen2 Modules: NZM2328 TI
The genes in each of Tables 27-1 to 27-48 can be used as effective biomarkers for classifying the LN disease state of the patient.

TABLE 27-1
Module 2.33

GM906, HADH, MGAM, SORD, DMGDH, SCP2, SHMT2, AK4, SLC10A5, PM20D1,
PBLD1, BHMT2, AKR7A5, TRAP1, DHFR, PRODH2, GM8765, ABAT, FGGY, NEPN,
PBLD2, APOO, TLN2, ADHFE1, AQP1, KHK, ACAA1B, MCUR1, CLEC2H, CGREF1,
SUGCT, D10JHU81E, PDE9A, GCSH, GGT6, CRYL1, OXNAD1, ND4, PLEKHA6,
CLDN4, LCN2, CHAC2, SBNO2, KSR2, SAMD5, CMAH, FAM210A, SLC22A4, COX20,
SCRN3, NDUFAB1, CLDN7, GATB, BMP7, TSPAN8, TMEM42, SEMA4G, TACSTD2,
CAT, SECTM1B, ACAA1A, TUBB2B, ZC2HC1C, PDCD4, SUFU, SLC39A5, SLC33A1,
CYP2D40, IL6RA, SMS, FGF1, MAVS, RBPMS2, PCSK5, FANCD2OS, CHCHD10,
PRSS8, TUBA4A, MAL2, FAM120C, GPHN, STXBP5L, APOA2, SLC6A4, GHR,
TANGO2, MACROD1, PPP1R12A, AAMDC, SLC16A9, TRPV5, NDUFS7, FN3K, SNX33,
GPR137B, SLC16A2, OSBPL6, SLC9A8, PITPNC1, IVD, HNMT, DNAJC6, DIO1,
2310061I04RIK, RMND1, GABRB3, TMEM70, MOCS2, PXDC1, IGFALS, ASPN, TRPV6,
PRPS2, NICN1, SDPR, TST, TOPORSOS, PCDH17, CYB5D2, DCLRE1C, GTF3A, ACO1,
MPV17L, EPHX2, GM4787, GREB1L, PCDH7, FAM60A, HIGD1A, COX6A1, NOVA1,
AP3S1, ZFP654, OLFR1487, ITGA7, TMEM205, CCDC189, ALDH5A1, FADS1, NAT2,
MURC, TM6SF2, MMP13, RBKS, GPRC5B, ESP8, GPD2, IPCEF1, NKAPL, OLFR353,
CYM, TAS2R108, SUCLG1, PGM2, MRPS35, INVS, METTL7A1, ATP8B1, OLFM3,
DYNLL2, LAMB2, GFM2, ACSF3, 2-MAR, SLC28A1, VEGFB, GM904, SQRDL, CIT,
ZFHX3, GOT2, TMEM135, PRKG1, 8-SEP, GULP1, FAM57A, BTG3, GABBR1, LCORL,
NDUFAF7, TMEM87B, 1110001J03RIK, GM561, CPT1B, SLIRP, ASAH2, NAALADL1,
TMEM218, DUSP11, UMPS, ITGB3BP, OLFR1359, DMP1, MYCT1, VMN1R87, LY96,
THBD, PGAM5,

TABLE 27-2
Module 3.35

SLC22A28, LRP2, CUBN, ZFP960, SLC22A2, FBP1, PAH, CYP2D26, SLC22A6,
SLC22A22, LRPAP1, SLC22A8, SLC6A20B, F13B, TRP53INP1, ANPEP, TCN2, ALPL,
ASS1, SPP2, MIOX, BC089597, 4833439L19RIK, METTL7B, A1CF, UGT3A1, PMPCB,
LACTB2, AADAC, CAR4, PCCB, HAO2, HGD, FAHD2A, SUSD2, SLC34A1, LPAR3,
C2CD2L, SLC1A1, UQCRC2, AGXT2, GLB1L, PTER, SLC13A3, CES1E, BC026585,
GATM, MYO7B, GSTZ1, RASD1, ACMSD, FMO1, PPL, SLC22A30, CIDEB, SLC2A2,
AOAH, ADD3, PYCRL, OIT1, VIL1, CYP2J5, TKFC, PCTP, TMEM139, AKR1A1,
ARHGEF37, UGT3A2, CISD3, 4931406C07RIK, TBC1D15, ACAA2, 0610011F06RIK,
LOX, ABHD3, SLC12A3, CLDN3, PLET1, IDH3G, HSD3B4, GPD1, DDAH1, CAR12,
GSTP1, DDIT4, SCEL, BC021785, PECR, REV3L, ZFAND5, JMJD8, D630023F18RIK,
TMX2, KMO, SUSD1, PPIF, SULT1C2, ST14, PFKFB3, SLCO4A1, ACNAT1, GLYAT,
NDUFA6, PGAM1, IGFBP5, RDH16, SRR, HAAO, HSD3B2, PCX, ACY3, CLCF1,
GCNT1, APOA1BP, BCAM, EPCAM, NPL, GGT1, LRRK2, MUC1, SDR39U1, CALML4,
CPN1, UGDH, CHCHD6, FAM151A, SLC51A, ARG2, INF2, RGS1, SLC4A4, SLC12A7,
GAB2, KIF21A, SLC25A15, TMEM116, WFS1, MACC1, SERINC2, CLDN10, DHRS1,
SAMD4, L3HYPDH, SLC5A9, SLC23A1, NDUFB5, TSPAN7, PIK3IP1, CERK, BCL2L11,
SMIM4, AI464131, POMT2, SMIM20, ENO1B, ERP44, CDHR5, JAK2, DECR1, SESN1,
LAP3, MRPL37, HIGD2A, MSS51, MRPL42, ODC1, SPRR2F, PCBD2, ELOVL2,
HIST1H2BC, PAQR9, GTF2I, TRPC1, PPP1R15A, LRP3, AR, PAX8, AMACR, AMN1,
CD46, CORO6, MASP1, DPEP1, SLC26A4, PLCB1, UEVLD, CAB39L, VKORC1,
SLC6A20A, HIST1H2BK, NABP1, COTL1, 2610507B11RIK, RAB11FIP1, EPRS, KDR,
SLC9A3R1, B4GALT6, MLXIP, AMY1, STIP1, ARRDC1, DGKH, ABCA6, ANGPT1,
HOXB5, AGO4, TGOLN1, ABCA3, NDUFV3, CYP4F17, ZC3H6, ADTRP, LDB1,
LMBRD2, SCPEP1, PLPP2, TMPRSS2, UBXN2B, NBEAL1, SLC44A1, FUCA2, SDC1,
TMEM33, IGFBP4, ILDR2, TFPI2, LMBR1L, MOB2, LAMA5, EPOR, L7RN6, APOD,
ZFP750, ALDOB, ITGB4, CDC25B, TLDC2, PROM1, MEP1A, ENPP3, FAM174B,
CCDC141, MAPKAPK3, RASGRP3, POLN, SELT, 4930402H24RIK, C1RL, TSHR,
TACO1, EID3, SLC3A2, VSIG10, TMCO1, SCAI, HOXB3, MLX, THEM7, FXYD6,
MTTP, FGFR1, ADORA1, GOLT1A, CPOX, MFGE8, GPR157, TAS2R143, SAP30, ADH7,
FAM110C, MPPED2, MED29, JAM3, ZFP97, 0610010F05RIK, YIF1B, GC, SULF2, MT1,
ARL4D, GNPNAT1, ABCB1B, FAM83B, SLC6A13, IFT22, REN1, RFC2, GPT2, BCKDK,
PHPT1, RNF169, MAP3K7CL, KCNJ10, CDH1, PKLR, STEAP2, IMP4, PPPIR16B,
SOCS2, ANKS4B, SLC37A1, GSTA4, COPZ1, PLCD1, TXNDC15, ACE2, TSPAN15,
ACP5, 0610007P14RIK, HTRA1, EXOC3, CDO1, PFKFB1, SCNN1A, TGFB2, PROC,
MED21, DBNDD2, SETD7, GSN, MAP3K3, HEXB, NSMCE2, VEPH1, CRY2, SFR1,
HTRA4, PAPD7, FAM174A, FAM208B, GALNT15, ZFP369, PIGQ, CES1G, PROB1,
LGMN, RETSAT, ACVR2A, SLC29A3, ZFP799, LAMA1, FLRT2, CD300LG, SLC5A1,
ZFP36L2, PCDHB17, TXNL4B, CNDP2, PEG3, 1110012L19RIK, XRCC5, AF529169,
PACS1, AP2A2, ENO1, SMAD2, GLT1D1, LRRC31, ETAA1, TAMM41, EMC2,
MAP3K15, GDPD1, RRAS2, FABP7, PRKAB1, DHRS7B, HEY2, TMEM14A, TMEM71,
LDHD, SMLR1, PLAU, KCNH1, PLA1A, SETBP1, CBR1, ATG4A, ZFP953,
9130008F23RIK, AQP6, LINGO4, SLC50A1, CES1F, CHST15, TMEM150A, ACADL,
SERPINB1A, KDSR, IGFBP1, ST8SIA6, SOX7, INPP5K, SEPSECS, DHRS4, SLC4A9,
GNA14, UPK1A, CXX1B, TAS2R126, AKR1C19, BDKRB2, IGFBP3, PAN2, OLFM4,
PHF7, PNO1, RABAC1, CPB2, PRR5, SLC25A20, CRY1, MAOA, PCDHB12, OLFR920,
ZFP273, AIM1, SEPP1, PRCP, LRP11, CD59A, COQ3, ARMC12, EPB41L3, EPS8L3,
TFAP2A, DCUN1D5, ATP6VOD2, POLR3K, OLFR1445, RMDN2, VSIG10L, TERF1,
HESX1, CTDSP2, SIK1, GSTP2, DMRT2, STC1, CLDN8, GM5901, RHEBL1, GSTO1,
PKDREJ, GM12248, 1700024G13RIK, 4930557A04RIK, HOXB4, GCNT4, HDAC4,
GPR33, SCNN1B, IFRD2, NUAK2, SH3BP4, OXCT2A, ADARB1, COX5B, PHYH,
TEAD2, SPSB1, AK2, APOB, SIAH2, ZCCHC11, LYPD6B, C1QBP, SLC35C2, TACC1,
STK16, AFG3L2, MARVELD1, SPINT1, DNLZ, PMPCA, MANBA, GAS2, PTPRK,
P2RY2, USH1C, KIF3C, MYRF, NLGN2, NDUFS5, 2810474019RIK, PARK7, TRPM4,
FAM213A, MRPL27, MTMR4, METTL15, CRAT, SLC43A1, DENND1A, TNRC6A,
HAUS6, SLC38A9, FBXL20, ELL, DARS, CLCN5, GALC, UCK1, 1110008F13RIK,
ERLIN1, BHLHE41, MGEA5, PIM1, HECA, RHOU, MTCH2, POF1B, METTL1, TLE3,
PRDX2, NAALADL2, STIM1, USP43, EIF2AK4, ACOT4, SOWAHC, SMG7, CLIC5,
ZFYVE1, ZBTB4, OSBP2, USP10, CYP2S1, TMEM39B, CETN2, G6PC3, MARVELD2,
GTF3C6, NTF3, DCAF6, GSS, HDGFRP3, NOP2, B630005N14RIK, TNS2, PHF1, PARK2,
ACYP2, CBLC, WDR72, GM8439, ESRP1, CTLA4, COG6, SZRD1, RASGEF1B,
PRELID1, SLC35B3, RCAN2, YEATS4, QTRT1, STK39, RALGAPA2, NHEJ1, SCUBE3,
ATG10, ITFG1, CYP39A1, USP22, GM2A, 2210408I21RIK, KLF2, HUS1, TRAPPC1,
OFD1, NEURL1B, ADAMTS9, ZFP521, MTX3, ZFP119B, SPCS2, PPP2R5E, MLST8,
MAPK1IP1, CCDC84, OCIAD2, HOXC10, IER5, TAF5, DPCR1, DCBLD1, VCPIP1,
MAGEA6, OLFR1391, SNAPIN, GML,

TABLE 27-3
Module 3.36

HABP2, DAO, ENPP6, SLC7A7, GABRP, UGT2B38, TMEM27, CSMD1, MMP7, AS3MT,
SOD3, FXYD2, SLC1A5, HCAR2, IGSF11, IDH1, TPI1, PSMB5, MGST3, TPP1, IDH3A,
PLLP, UGT2B5, SLC30A9, S100A1, TMPRSS4, CHDH, CLDN2, TRPV4, DCPS, PDIA5,
TREH, EIF4E, PTS, SLC38A3, TIMM22, SLC35E3, ALDH3A2, KCNK5, IHH, SIM2,
ATP6V1C1, UBE2N, SORBS1, PTCD3, RRAGA, ATP6V0E, ALG14, 4930550C14RIK,
DPP4, GLTSCR2, RPE, 1700017B05RIK, IDI1, BYSL, SDF2, HAUS1, BCAT1,
4930563E22RIK, PARP6, SLC4A5, SPC25, GGH, CPT2, CDH16, CPPED1, RPL7L1,
NPY6R, TSPAN31, ADPGK

TABLE 27-4
Module 4.44

HIST1H2BL, HIST1H2BN, CHORDC1, PDIA4, HSPA5, NPHS2,
CALR, HSP90B1, HIST1H2BJ, DZIP3, STT3A, MAP2K7,
SAT2, TOP1, THAP3, CECR2, SGTA, NAIF1, WSB1, EZH1,
CIRBP, HIST1H2BA, INTS3, HIST1H2BF, UBAC2, NOLC1,
KLKB1, CYS1, NOL10,

TABLE 27-5
Module 5.47

HIBADH, NOX4, ALDH7A1, ALDH8A1, MUT, MDH1, CSNK1E, ACAD11, KCNT2,
MCCC1, KYNU, AASS, ALDH6A1, ITPR3, ASL, PEAR1, PDZD3, ATP5F1, TCAIM, FH1,
EPHX1, DBT, PTGR2, BST1, GCDH, KLRG2, HAP1, DEB1, TIMP3, ZDHHC15, ANKIB1,
FKBP4, 4930523C07RIK, HAGH, COL12A1, SOX4, CMBL, CERCAM, DLST, PDP2,
SFXN2, PCYT2, PDE3A, RAB11FIP3, HADHB, STK17B, SLC27A2, DKK3, RBM46,
XDH, SLC25A10, HNF4A, SHMT1, NDRG3, PAMR1, CAPG, SLC43A3, PDHB, CD33,
BCKDHA, NDUFS2, CPT1C, CHPT1, IMMT, 1700024P16RIK, GJB2, G6PC, JAM2,
PHOSPHO1, SCARA3, MFSD10, FAM205A2, IL4RA, HOXB9, CHST11, CXCR4, EFNA4,
NIPAL2, OLFR1396, NPY1R, ZFP706, PHB, HSPB2, NUP37, SLC37A4, INAFM1,
SLC25A42, BEND5, PLA2G7, ATP6V1B2, DOK1, ARHGEF38, KCNJ15, CRISPLD2,
REM1, BC039771, ENTPD1, 9430020K01RIK, MFSD7C, RHOG, TEX2, CORO2A,
SUCLA2, ATAD3A, PTK2B, PIANP, GXYLT2, FMNL3, PLD2, RNF180, CAPN5, LIN7A,
HPN, FASTKD1, MLYCD, MATN2, RASA4, PIK3CG, CTTNBP2NL, ATP2A3, NCOA7,
SH3TC1, PPT2, SLC25A16, ARHGEF17, ZFP703, UTRN, MMGT1, UGT1A2, TPCN1,
CDK17, F13A1, STUB1, LYRM9, COX11, HSPAIA, CRIP2, MINPP1,

TABLE 27-6
Module 5.48

SLC26A1, FUT9, ACAT1, PDZK1, GM13157, GALNT11, SLC16A4, PCBD1, FADS2,
ACSM1, SMIM24, DMTN, SLC5A2, ACY1, CHRNA4, ALDH4A1, TGIF1, CAR14,
LAMTOR5, SARDH, DPEP2, CYP2J11, SLC16A12, UQCR11, GNPDA1, XPNPEP1,
PRLR, ACSS2, TXN2, SLC34A3, AFM, UNC5C, KL, SLC36A1, PTH1R, SLC18A1,
APEH, DNASE1, BNC2, MTHFD1, F3, FADS3, SSH2, SLC25A21, PLAUR, WDR26,
DDO, MRPS36, FOSL2, DNAJC12, FGFR4, VEGFA, VWA5A, CNNM2, ND3, AKAP12,
SPATA6, CEBPD, THSD7A, XYLT1, USP53, CCKAR, L2HGDH, BTG1, CELSR2,
MBNL2, SCOC, NNT, WASF2, EHF, AKR1E1, TGFB3, XYLB, ANKRD44, OAT, RERGL,
SLC7A8, ATP5O, PYROXD2, ZFP52, CCPG1OS, 4933411K16RIK, F2RL1, PAPSS2,
DCDC2A, NADSYN1, DLEC1, DNAJC5, 1700040L02RIK, NFKBIA, NFYC, PTPN22,
B4GALNT3, MRPL15, PTGFRN, INPP5J, TMEM86B, ST3GAL4, ZBTB42, EML4, SAT1,
RHCG, CCND1, PHF20, NPAT, SEL1L3, TMEM206, SMC5, AMPD3, MTFP1, RELL1,
RGL1, PLCB4, INSRR, AHNAK, FLRT1, CAR2, SLC25A19, KIF12, SORBS2, UBP1,
QSOX1, FAM76B, ESRRG, AMT, SHANK2, ADGRG2, SUPV3L1, SLC22A17, GM15446,
RHBG, CAST, OXGR1, MXRA7, RAPGEF5, CAMKK2, MXD4, S1PR1, MIER1, L1CAM,
CCDC82, NDUFAF1, POMT1, ETHE1, PREX2, FBXO4, PDZD11, PHF13, EFEMP1,
ANTXR2, VOPP1, MMACHC, MID1IP1, PPP1R2, COQ9, S100G, CAR5B, TRIP10,
PRDM4, SLC35E4, KLF3, RASL11A, MS4A4A, RASL11B, ACO2, THAP4, RCAN3,
SMPDL3A, RAB11FIP4, PAPD4, ANXA7, FAM199X, WBP1L, CBLB, ACER2,
PLEKHA2, ABCA13, 5730480H06RIK, DUSP10, DCUN1D2, MLXIPL, UBE2A, TSPAN1,
RAD18, TMEM171, AW209491, RANBP3L, TXNIP, REEP4, DENND4A, PHF20L1, NPY,
HIVEP1, LZTR1, APLNR, SEMA4A, BC025446, POLR2K, DCLRE1B, GADD45A, PLEC,
DDX26B, SLC9A2, ARHGAP18, KCNJ1, RFNG, EMB, BC005561, RC3H1, ACAP2,
TTC30A2, EDNRB, MFSD7B, SLC44A3, SLC4A7, CLNK, KLHL6, IFRD1, PHF10, MAL,
EFCAB10, FAM222A, MTHFR, CBR3, GIMAP9, ATP6V1B1, FXYD4, YBX3, PGF,
GRPEL2, DPP7, IMMP1L, MRPL39, SLC7A13, RNF125, KCNC2, LETM1, ANO9,
AGPAT1, HIST1H2BB, MZB1, MYLIP, GM13152, ANGPTL7, STON1, SERINC5, SOX17,
MTHFD1L, LRRC20, GALNT4, GTF2F2, CCNL1, OLFR417, FABP5, ACAD8, ATP5C1,
UNC13B, NDUFA12, COX8A, TRAFD1, SGPP1, LRRC16A, NOP10, TYMS, PROZ, AK3,
COX7B, DALRD3, RAB3IL1, ABI1, C330018D20RIK, DHRS11, REX2, PRKCZ, DHCR7,
EPN2, CLCN6, MED13L, CYP2U1, COX15, CHIC2, UTP14B, KRT7, NFAT5, NIT1,
HDHD2, SLC41A1, CCNG2, TRIM24, 0610009B22RIK, ZHX2, PDE2A, CDS2, TOPBP1,
DNAH7B, SMAD4, CYLD, ZAK, SERPINB6A, SIAH1A, GM5622, SNX24, ZYX,
IRF2BPL, PPM1H, SCARB1, CHFR, CTSL, FN3KRP, INTS10, BC003965, FAM124A,
MYBL1, NOA1, SLC1A2, SMOC1, SLC16A5, PET100, DARS2, ABHD15, DDIT4L,
SLC25A34, ZFP691, UPP1, OLFR43, OLFR1362, BBS10, GZMB, MAP10, ADRA2C,

TABLE 27-7
Module 5.49

GYS2, SLC22A29, ASPDH, UPB1, AFMID, DCXR, AQP11,
SLC22A27, ACAD10, TGFBR1, FAM129A, ACP6, CDKN1A,
PDGFRA, DYNC1LI2, MORN2, SCIN, SASH1, ADAM10,
DHTKD1, 4931406P16RIK, CPD, MIS18A, ADAMTS5,
ARL15, TPP2, COQ5, PLAT, SMC4, AKR1B7, PDGFD,
GALNT9, NOXRED1, ZKSCAN3, H2AFB1, GALNT7, MASTL,
TNFAIP8, ECHDC2, IDO2, MRPS21, SHC1, MYEF2,
TBL1X, TMEM88B, CTSO, MAP2K6,

TABLE 27-8
Module 5.5

MYO1B, ITGA3, ASAH1, NLRP6, CTGF, CAMK2D, SERINC3,
CYP20A1, CXADR, SEMA4B, PDK4, ABCASA, SEC14L1, SCARB2,
XRN2, PLEKHH2, ANGPTL4, FAS, KLHDC7A, RBFOX2,
TMEM43, PGD, POSTN, TET1,

TABLE 27-9
Module 9.62

SELENBP2, DDX58, RTP4, EIF2AK2, OASL2, CMTM3, PARP14,
TRIM12C, SLC34A2, SELENBP1, DDX60, IGTP, STAT2, DHX58,
IRF7, DPYD, IFIT1, HACD4, IFIH1, GBP7, PPM1K, GM8267,
FMO2, ZMYND15, FBXW17, CXCL1, FAM171A2, ISG15, TLR3,
SHISA4, NDRG2, SLITRK1, PARP9, HMGN5, ADAR, IFIT3B,
IFI35, GABARAPLI, MLKL, 1700049E17RIK1, BCAR3,
IFIT3, SLC35D1,

TABLE 27-10
Module 9.63

9930111J21RIK1, C1RB, OAS1A, C1S1, MS4A4C, H2-AB1, H2-EB1, CD74, MNDA,
OAS1G, PYHIN1, H2-DMB1, ADGRE1, SAMHD1, BST2, IFI204, OAS2, CCND2,
LGALS9, TRIM30A, SCIMP, H2-AA, TLR9, ZBP1, XAF1, SERPINA3G, LSP1, SP100,
FCGR1, GM5431, CXCL9, HERC6, USP18, PYDC4, B2M, MS4A4B, GM11710, NRP1,
IFI27, C1S2, CX3CL1, NAV1, CFI, UBD, FOLH1, CLEC5A, IFI27L2A, LGALS3BP,
EFEMP2, PIK3R1, H2-DMA, HSD3B6, PHF11D, ARHGDIB, CD72, FAM111A, CXCL10,
MARCKS, MIA2, CAMK1D, LAPTM4B, BC094916, SMIM3, PTPRO, RSAD2, ALDH1L1,
C1RA, AQP3, CCL12, A930007A09RIK, OLFR228, NAT8, PSMB9, MGL2, TSSK1,
PYDC3, IQCF4, ZFP189, 9930111J21RIK2, IL2RG, IFI202B, UBE2L6, GPM6B, GLIPR1,
SLFN5, GRAP2, ACTB, SRGAP2, IRAK4, CRTAP, PRKCDBP, IRS1, BAMBI, TRIM34B,
PMEPA1, SALL1, IFI27L2B, TRIM30D, CD69, DNASE1L1, KCNJ13, GRAP, CXCR6,
CCL20, DYNAP,

TABLE 27-11
Module 9.64

H2-DMB2, MS4A6C, CYBB, MRC1, MS4A6D, VCAM1, AOC1, GM12185, MGP,
MS4A6B, APOBEC1, SLIT3, IIGP1, AXL, ADGRG6, TNFSF13B, IGSF6, P2RY6, C1QC,
ALOX5AP, IFITM3, CCL6, NFKBIZ, CSF1, IL10RA, STAMBPL1, ITGAM, SERPINE2,
ICAM1, TGTP2, LYZ1, ELF4, TGFB1, LGALS1, FKBP10, DOCK10, FBLIM1, FCER1G,
PMP22, CSF1R, HCLS1, CRIP1, H2-M3, CCR5, RAB31, GPC3, TLR13, PSMB8, C1QA,
AKR1B8, PLEKHO1, PIP4K2A, CDK6, SIGLECE, CSTB, ESM1, GM21188, RAB8B,
CADM1, MMP12, TLR8, RAB32, ZC3HAV1, CXCL14, S100A4, LAIR1, PSMB10,
ATP6V0A4, ASF1B, MILR1, MEF2C, ADGRE5, FAM49A, NFKB1, DDX52, SLFN2,
REEP5, 1810011H11RIK, TLR1, PTRH1, IRGM1, CAR13, IL1F6, MEX3B, LRRC25,
RGS10, PF4, ATP2B4, H2-T24, GSDMD, CENPT, LPIN1, DKK2, H2-T22, MKRN2OS,
LRRC32, FAM101B, PPP1R14B, TMEM123, NRM, FAM134B, GSG2, MID1, CALCRL,
CDKN2AIPNL, WDR43, GPAM, IER3, LIG1, LYN, SPI1, NUP210, CORO1A, SRGN,
AF251705, IDH2, ARPC2, TIMP1, CBL, SHTN1, PLA2G4A, 2200002D01RIK, LPCAT2,
FPR2, MCM3, SLC30A2, A630001G21RIK, GM4951, CXCL2, TAPBPL, ELK3, SLFN9,
VMP1, GUSB, SRPX, SERPINA1E, ARSA, NUAK1, A930017K11RIK, CARHSP1,
S100A13, 1700020N15RIK, CD83, OLFR982, OLFR875,

TABLE 27-12
Module 10.66

CFHR2, CFH, SLC7A9, PIK3C2G, BPHL, GM7068, THNSL2, HYKK, SLC9A3, FOLR1,
LRRC19, AMN, NBL1, ABCG2, ASPA, SLC22A1, 2010107E04RIK, ACOT13, IYD,
LYPLAL1, KEG1, PDZD2, MRPS22, UQCR10, LGI2, BDH2, CLUH, KRT18, DAB2,
PSCA, USMG5, DGAT2, PDPN, SCRN2, SDHC, PIPOX, TMEM106A, PGK1, CLYBL,
KLF4, ALDH9A1, ACADVL, SLC5A11, SFXN1, SGK2, ST6GAL1, FAM163A, RDH19,
GPNMB, SERPINA3N, DHCR24, PTPN14, CFAP69, ISOC2A, SCN7A, ITGA2, UGT2B34,
RNASEL, SLC51B, RHOBTB1, FAM46A, MGLL, ME1, CLTB, ELF3, MTFR1, MDK,
DOCK9, PRKAA2, NME2, GM4788, RAB3D, GM16432, HSD3B3, ENPP2, TEFM,
ACADS, AGT, ABCG1, FABP3, PSMA4, KRT19, MME, ADGRA1, RGS16, SERPINA10,
CD302, MRPL45, PCDHB20, PTGIR, SYPL2, MPZL1, KIF1A, RSPO3, DECR2, FBXO40,
PTPRD, XPR1, OXSM, FBXO7, SLC22A23, PIEZO2, NDUFC2, CRYZ, NDUFB6,
SLC47A1, MSRA, FAM172A, GPM6A, CDHR2, SYTL5, MPV17, SORT1, AAMP,
SYDE2, STOML2, HSD17B10, DERA, BC034090, NKIRAS1, TRABD2B, RMND5B,
CRYM, PRPS1, PANX1, EFNA5, SFN, CHML, SPRYD4, OLFR624,

TABLE 27-13
Module 10.68

GALM, SLC22A18, NDUFA9, UQCRC1, GHITM, TMEM8, BPNT1, CHCHD3, ATP5B,
ETFA, REPS2, YIF1A, B3GALNT1, FAM195A, GDE1, ECSIT, RTN4IP1, FAH, GRHPR,
CYC1, NDUFAF5, GLYCTK, SLC25A39, COX7A2, CES1D, PLSCR2, ABHD14A, BID,
ALDH2, MFSD1, MRPL46, PNKD, SIGIRR, LRCH1, YARS2, ADAMTS10, SLC46A1,
NDUFA8, FASTKD2, MRPL22, NSDHL, ATP6V1E1, CYCS, NDUFS4, ATP6V1H,
HIST1H4I, NUDT16L1, TBC1D8, CRLS1, ALS2, MMAA, PEPD, TM2D3, ESD, SEC31A,
ATP5A1, SDHAF4, NQO2, ATPAF1, SLC31A1, TBC1D22B, SAMM50, RHOT2, GM8369,
CMPK1, NANP,

TABLE 27-14
Module 10.7

TOMM7, ETFB, MINOS1, NDUFA2, NDUFV1, LAMTOR2, D8ERTD738E, GM10094,
BSG, HRSP12, GNPTG, TIMD2, TMEM147, NME3, FUCA1, PSMG1, FAM132A, IFT20,
KHDRBS3, ETV5, SMDT1, SAR1B, SNAPC5, TIMMDC1, DUSP9, TMEM52B, CES2E,
CYP2C69, FECH, HSD11B2, ETV1, NUDT4, AGTRAP, MRPL24, SNX27, CD164L2,
OMA1, MRPL17, CYB561D2, MND1, CYP2C68, EEFSEC, IL22RA1,

TABLE 27-15
Module 10.71

NUS1, ITGB8, GFM1, DNAJC13, ACADM, SLC16A13, HSPH1,
P4HA1, GPC4, SLC8B1, KLF5, CGNL1, SLC16A10, TYW3, ITGB6,
VWA8, RBM11, 1700034O15RIK, EXOC6, PDE1B, MAML3, NT5C1A,
CCDC47, TEK, SULT5A1,

TABLE 27-16
Module 10.72

NDUFA4, NDUFV2, NDUFC1, OXCT1, GCHFR, ABHD14B, SUOX, GLOD5, LPL,
MIPEP, NIT2, ECHS1, AP1B1, CYB5A, IGFBP2, ARHGAP42, SLC16A7, NUBPL, MKKS,
GM3873, ACSS1, NFS1, NDUFAF4, MPST, CYB5B, WNK1, ABHD17C, MTIF3, MPC1,
RUFY3, AHSA1, ADGRF1, PCDHB8, NDUFB4, PTRF, TMEM143, ARHGEF39, MR1,
CHCHD1, RPRM, SPHK1, TBX2, PYURF, CD300LF, OCIAD1, BC100451, PEX19,
HHIPL1, GPR173, ACER3, CCL17, CCDC3,

TABLE 27-17
Module 10.73

2610528J11RIK, ACOT3, VWCE, CCDC107, TTC36, AKR1D1,
ENTPD8, ESYT2, SLC2A12, FXN, HEMK1, CML1, ZMYND10, CKS2,
MGAT3, RBPMS, HIVEP2, ARVCF, MOB3B, TECR, NHS, LZTS3,
NME4, FTCD, TYSND1, PGAP3, PRIMA1, BMP1, CNNM4, PPOX,
XKR7,

TABLE 27-18
Module 12.76

CLCA3A1, GM7697, GM21119, FLNA, ANXA2, GM9495, FAT1, TAGLN, ACTN1,
RRAS, TUBB5, TPM1, ADAMTS1, HPD, PEA15A, MYADM, ITGA5, EMP1, ACTA2,
YWHAH, TUBA1A, ADAMTS2, MYH9, ANO6, UCP2, LEPROT, CLCA3A2, ANXA5,
NRG1, HSBP1L1, GSTM7, SERPINE1, GBP3, MCAM, CYP1B1, PXMP2, TAGLN2,
ANXA6, LOXL2, TAX1BP3, CEP170, GNB1, PARD6G, MYL9, SLCO3A1, SQLE,
ITM2C, DLL4, CAPNS1, ADSSL1, NES, ATP11A, NCS1, GBP2, PTPN9, GPX8,
4430402I18RIK, MFAP3, CD248, GOLT1B, TRIM47, PLIN2, CYR61, LAMB1, NKAIN1,
TMEM252, DYSF, PAPPA, HACL1, DEFB1, CABLES1, ARRB1, DDIT3, FIGF,
SLC25A35, CAV1, EXT1, TPM2, SEMA5A, CCDC86, PIK3R3, ENG, KCNH5, NKX3-1,
TUBA1B, TAS2R119, IL17RB, TSPAN6, CLDN34D, FGA, OLFR283, RBM4, GM5741,
HEY1, STRA6L, UCK2, FGF9, GM15319, GNAI2, ABCD4, PRRX1, TMSB10, SLC39A6,
WDR1, LIMCH1, LAYN, AGFG1, ASNS, AIDA, DCLK3, HSPG2, RAP2A, OXLD1,
FAM13B, CASP8, CALM1, ACSL5, PERM1, DUSP5, EHD2, HEG1, P2RX1, DNAJB9,
REPIN1, VAMP1, ZFP648, C1QTNF9, RGCC, OLFR993, OSBPL3,

TABLE 27-19
Module 13.77

SDHB, GM11128, FAHD1, TINAG, SLC3A1, NDUFA10, UQCRB, NDUFS8, TCEA3,
UQCRH, HAX1, LYRM5, NAA38, NDUFA1, SOD2, ATP5K, ARL6IP1, HINT2, SMIM22,
PPA2, ASB9, CCS, GM5617, EMX1, NDUFB9, ATP5H, POLDIP2, TMEM174, UBE2U,
GCAT, NIPSNAP1, NDUFA5, ALAD, MRPL28, HMGCL, NME5, COA6, NMRK1,
RASL2-9, ACBD4, MRPL13, IAH1, GRTP1, CISD1, TRIM7, MRPL47, MAGI2, TPRKB,
GM4477, RSPH3A, FRRS1, APOOL, COASY, DDT, FMO4, SEC62, SOD1, KNG2,
ECHDC3, SNX6, DEFB29, SIAE, 3830406C13RIK, PSMD3, AGMO, SRD5A2, COL18A1,
MAPT, ICT1, PITRM1, PON3, PCLO, ALKBH7, GM13306, MARK1, APLN, MCCC2,
EGLN3, HLF, EAF2, CASR, AKR1B10, SERPINB9, NPRL2, STXBP4, GUCA2B, TTC7B,
VDR, GPATCH8, TTR, NDUFA13, PIM3, NDUFAF3, HNF1A, MEIS1, TXNDC17,
METTL7A3, GPX1, C1QTNF7, NAP1L5, CCBL2, PLA2G5, FAM58B, A930033H14RIK,
RSPH9, SURF1, PAM16, MRPL4, SLC44A4, THPO, PVRL3, MRPL48, BLOC1S1,
CHCHD2, SIPA1L1, CS, D7ERTD443E, ATP5D, TRMT2B, KLF12, EBP, KCNJ5, ZFP740,
FUS, RP2, PDXK, ZFP944, TMEM53, SLC13A2, FBXW15, QDPR, CPEB1, TMEM72,
CRLF3, MRPS28, FAM217A, WDR18, SYT17, SLC35F3, ALS2CR12, 9230112D13RIK,
CCDC122, 2300009A05RIK,

TABLE 27-20
Module 15.83

GM20746, TIMM17A, GTF2H3, IL10RB, ARL8B, FTL1, MED28,
MAN2B2, PSMC5, GJB3, SREK1, TRAPPC4, CDK10, CD163, CCNH,
GPR141, 9130023H24RIK, TMEM238, TREM1, DHRS7, FUOM,
BC030336, CFDP1, AGK, GM11564,

TABLE 27-21
Module 15.84

COA3, NME1, MRPL2, TIMM13, UCHL3, COPS6, CNPY2, N6AMT2, TMEM14C,
TMEM126A, AMDHD2, DYNC2LI1, TSTD1, GGA2, TGM2, POMP, RPP14, CCDC58,
GSTT1, SNX16, RENBP, AMD2, GLMP, REXO2, CIB1, FAM149A, FZD8, FAM136A,
SOSTDC1, PCP4, OLFR1196, FOXC1, SEC23B, ERGIC3, WDR83OS, E430025E21RIK,
RCE1,

TABLE 27-22
Module 17.95

MRPL55, COX7A1, DNAJC15, NDUFA11, HSPE1, SLC10A6, COX5A, COX6B1,
ATP5G1, BC031181, SFRP1, BTG2, ATP5E, BCS1L, TUFM, MCEE, HACD2, MRPL34,
PTBP2, MAP3K6, UQCRQ, FKBP5, SCLY, DAD1, PSTK, GRPEL1, ROGDI, ZBTB16,
NQO1, PRDX4, SUCO, TSC22D3, MKNK2, TOMM5, MRPL21, TBXAS1, PGAM2,
BC022687, TLR6, RAD23A, KDELR2, CYBA, CCDC90B, ZDHHC20, TMEM179B,
4921507P07RIK, TMA7, SCN3A, D6WSU163E, ALG3, SGTB, PER1, TNFSF15,
TMEM242, SMPD1, AQP2, 1700021F05RIK, RNF133, NINJ1, CRYGA, ALG8, MRPL10,
MSRB2, GNG11, FAM117B, PSMG2, PHLDA1, METTL23, GIPC1, PRKAA1, GLO1,
STAP1, CHID1, BC051142, ZC4H2, ALYREF2, TMEM138, 1500011B03RIK, OCLN,
ZNHIT1, MRPL38, BC004004, SPOPL, HYAL2, FCGR2B, PARP4, ST3GAL3, NFIA,
TMEM175, CMC2, 1810037I17RIK, KLF13, FOXO3, SNRNP70, ZSCAN25, USP54,
SF3B6, FAM173B, APBB3, HS6ST1, APLF, GM6176,

TABLE 27-23
Module 17.96

NDUFB7, MIEN1, NDUFS6, COX14, MRPS15, PGM2L1, CCDC120, ID2, EMC9,
LAMTOR4, ANAPC13, SLC35F1, 1810043H04RIK, IRAK1BP1, 2210016F16RIK,
SMIM11, 1700109H08RIK, TRAPPC2L, SUCNR1, TCEB2, H2-KE6, FBXO11, LYRM2,
CUTA, MRPL16, APIP, MRPL36, RPL31, DDRGK1, CMTM8, PHLDA2, ARRDC3,
AGTR1A, ZMAT5, NHLRC1, NDUFAF2, TMEM208, SH2D4B, TMEM141, SNRNP27,
ENOX2, SELM, TTC32, SIVA1, VSIG2, CPEB2, TK1, TMEM220, RNF32, MOCS3,
SVOPL, MSH6, UQCC3, ADRB1, SPCS1, MCTS2, COMMD4, MYEOV2, TGM1, ZFP639,
SERF2, FKBP11, OLFR520, TSPYL1, HNF4G, AP1S3, CES2H, LCE3A, MED10,
VMN1R19, COX17, RWDD4A, NEK4, DPH6, GATC, HSD17B11, MRPS16, MRPL11,
TOP1MT, PRORSD1, IFT27, SLC17A4, POLR2F, SNRPF, 2700097009RIK, B3GALT5,
HSCB, SLC39A10, TMEM237, TBCA, FRS2, CPTP, DGKG,

TABLE 27-24
Module 17.98

CDKN1B, CREBRF, RMND5A, HBP1, KLHL24, PPP1R11, ANKRD53, STAG2,
1600002K03RIK, GCLC, RGL3, ERBB3, RASSF6, PLCG2, TMEM88, IP6K2, ZFP958,
RCAN1, COL4A3BP, SEC22A, FNBP1L, FCHO2, EML5, ZBTB44, DTX3, OLFR1392,
CHD2, ACTR1A, PCDHB10, EPS8L2, PALLD, BZW1, EFCAB8, OLFR630, DYNLL1,
DYX1C1,

TABLE 27-25
Module 18.1

GM2237, GM2897, GM3264, INPP5D, GM10406, GM5796, GM3591, MYO5A, GM3500,
GM3667, CDH6, IRF4, GM2956, RNF150, GM3383, LTBP2, NCAM1, SCUBE1,
SLC4A11, PTGDS, GM3317, P2RY13, SPINK8, ADK, GM3696, GM3558, AIM2, CCL28,
FOXC2, GM3636, AGAP2, 2610042L04RIK, LYST, CNRIP1, TFB2M, GM9994,
SLCO4C1, SAMD9L, LYNX1,

TABLE 27-26
Module 18.101

C3, H2-K1, NEURL3, PLAC8, TNFAIP3, PARP12, H2-Q4, CD44, CD86, AI607873,
RUNX1, CASP4, H2-Q7, TAP1, CALB1, STAB1, MVD, LPXN, C2, CTLA2A,
5031414D18RIK, PPM1J, JCHAIN, SLA, SLC8A1, MS4A4D, ARHGAP25, GALNT12,
CLEC4N, MDFIC, GALNT18, EFHD2, HBEGF, ZFP830, XBP1, TMEM150B, GBAS,
FERMT3, XLR, C5AR1, PINX1, AI429214, S1PR2, CTLA2B, TMPRSS12, RRAD, FMOD,
ATF3, LRRC26, TOR3A, H2-T23, LHFP, H2-Q2, NLRC5, D630039A03RIK, SLC25A12,
FLI1, RAB3IP, ICOS, FAM234A, FRG1, ZWINT, 3830403N18RIK, SIAH3,

TABLE 27-27
Module 19.102

MPEG1, TYROBP, MS4A7, ITGAX, FYB, C3AR1, UNC93B1, CYTH4, CTSS, CCR2,
GM11709, APBB1IP, IKZF1, DOCK2, SLC9A9, SLC15A3, IL1RN, THEMIS2, HCK,
PLA2G15, REL, PTAFR, MS4A14, SLFN8, RASAL3, LY9, SIGLEC1, RASSF4, CD4,
ARHGAP4, PTPRC, LAT, GLDC, CIITA, KLRB1B, BTK, PLXNC1, PTPRE, LILRA5,
SLAMF6, TESPA1, SKAP2, LYL1, PRODH, CCL7, KLRD1, KLHL33, IL27RA, FOXS1,
CYSLTR1, KLRA17, FBXL21, KPNA6, NCF4, GMFG, NAIP6, PRKCB, MFAP4,
GM4980, PILRB2, ELMO1, FAM105A, GAL3ST1, PSME2B, APOL7C, RIPK3, ELMOD3,
LCAT, GM21645,

TABLE 27-28
Module 19.103

GM14548, IL1B, PIRB, ITGB2, PIRA6, CLEC4A1, SIRPB1A, PLTP, PLEK, CCL2, FPR1,
GM15448, APOE, BCL2A1D, BCL2A1C, CCL8, PILRA, CLEC4A2, CD8B1, LILRA6,
TREML4, TNFAIP8L2, CSF3R, HP, ADGRE4, OLR1, UPK1B, GM5150, TNF, ATP8B4,
ZC3H12D, GNA15, CSPRS, SLC5A10, SLITRK6, FGR, OLFR605, TRF, SPRR2B,
SPRR2A3, COX6B2, GIMAP5, HCST, SELPLG, IRF5, AB124611, PPARG, CD300LD,
GLTP, GNGT2, BC028528, CLEC4E, EAR1,

TABLE 27-29
Module 19.104

CSF2RB2, LILRB4A, CD68, NCKAP1L, ARRB2, CTSC, LILR4B, SLC11A1, FXYD5,
FES, FAM49B, CD84, TMEM173, VSIR, PTGER4, TM6SF1, TNFRSF1B, CFB, NCF2,
GPR65, CSF2RB, MYD88, CD53, CCL9, SP140, CCR1, LAT2, ADCY7, CASP1,
PLEKHO2, LACC1, ADAM8, MFAP5, ADAMTS16, TNFRSF4, BAZ1A, GLP1R, CFP,
EDARADD, TMSB4X, 1700056E22RIK, ADGRF5, PLXND1, TRPV2, SYK, DOCK11,
NFAM1, FCGR3, TNFRSF1A, ITGA4, CTSE, MAP7, ADGRG5, ACADSB, ADAM23,
POT1B, MAFB, CD2, ETV6, DENND3, DOK3, CCR4,

TABLE 27-30
Module 19.105

CLEC4B1, RAC2, CLEC4A3, CX3CR1, GPR31B, PHF11A, LCP1, FCGR4, MNDAL,
EVI2A, MYO1G, LST1, CD48, PHF11B, NAAA, BCL2A1B, FGG, CD180, AIF1,
SLAMF9, FAM26F, CD52, PSME2, CD3D, CTSB, CLEC12A, TRP53, CLEC2D, IFI203,
TTLL12, WFDC17, ERH, CMTM7, SGMS2, CD300E, CYP24A1, C4B, IFI44, OSTC, LUM,
TIFAB, SNAI2, FABP1, CCL5, TMED10, PDGFB, HIST1H2BG, CHST2, GUCA2A,
IMPDH2, CTSW, IRG1, BCL6, CNNM1, SH2D1B1, SPN, SSR2, PRMT6, OLFM1,
PIK3AP1, ARHGAP30, GIMAP3, PTPN6, IRF8, NKG7, IFITM2, H2-OB, IFNAR2,
NUCKS1, CDK4, RAB3GAP1, DDX21, MFSD5, SLC5A3, FUT4, BCL6B, GAPT,
OLFR361, E2F3, CCR7, BC035044, RND1,

TABLE 27-31
Module 20.108

SLC38A2, ACOX2, NCL, FCAMR, SLC20A1, WFDC15B, 2610305D13RIK, ROCK2,
BCKDHB, PPP1R1A, UROS, ZFP600, ITGAV, PPARGC1B, GM13242, NUPL1, LRRCC1,
FAM107A, PHYHIP, SSH3, LNPEP, GLS, PPFIBP1, SLC12A2, ACSL4, DUSP15,
ERBB2IP, PLK3, CTNNA1, KIF2A, EGFL6, ZFP948, UROC1, PTP4A1, ZFP810, ZBTB9,
ARHGAP11A, ZFP442, SLC25A30, TOP3B, FAM135A, USP6NL, SDCBP, MOGAT2,
TUBB2A, RNF217, NBEA, ZBTB10, GM13247, GM10277, AZIN1, ADAMTS6, CAV2,
CD151, ZFP942, USP14, MSMP, PCDHB14, ATP11C, PTGER3, LIX1, NR0B2, MIA,
TEX30, YPEL2, ZFP937, OLFR1375, EPHX3, GM3414, SLC1A4, PDCL3, SLC12A1,
FZD4, CAR8, ZFP526, RAD50, PEX6, FITM2, AGPS, H6PD, RDH5, COG3, TFCP2,
TOP2B, MYH11, PUS10, TSPAN12, FBXL16, OLFR1490, APELA,

TABLE 27-32
Module 21.109

IL34, PLP2, C7, SYTL2, CPE, SMPDL3B, CHRNB1, TES, KLF6, CNDP1, GOLM1,
BSPRY, MYOF, LHX1, ECI3, PARP8, ANXA1, PVALB, LYPLA1, CLIC1, EGF, SNX29,
ACSM5, EXTL3, ACOT9, KLHL3, PDK2, GRN, NUPR1, GPX6, LRRC52, PQLC3,
SLC43A2, SH3BGRL3, CDH3, ELF1, CD24A, PIEZO1, PRRG4, KRT8, B3GALT1,
MAMLD1, TMEM181A, TSPAN17, YBX2, RTN4, CACNB3, UBTD2, SRD5A1,
ZDHHC14, RASSF2, SIRT3, PI15, CTSD, TRAM2, KIRREL, FRY, HSD3B5, MAOB,
HPGDS, SLC6A9, LRRFIP2, OLFML2B, ZNRD1AS, CBR2, SRGAP1, FOLR2, MPP4,
BCAT2, MAP1B, PRIM2, SLC7A6, GMDS, DNPH1, SEMA7A, RGS2, TEAD1, DUSP7,
PVR, SLC7A5, MAB21L3, SYN3, SLC22A26, EIF1A, TVP23B, HMOX1, TPCN2,
ATP6V1C2, CALML3, PFN3, HDDC3, MAP4, F8, TMEM144, APOLD1, TGFB1I1, NMI,
NRAS, CRLF1, LOXL3, DYNC1I2, OGFRL1, HOXA11, SLC25A24, TMEM165, RHOJ,
PDGFA, EPHA2, PCDHB21, IMPA1, MITD1, NLRP3, ARID5B, DUSP8, SNRPN,
TBC1D13, C1QTNF3, CPNE4, CCDC109B, MYC, LOC100041057, RAD1, ZFP146,
THRB, TXNRD3, ZFP768, HDHD3, GPR39, TTBK2,

TABLE 27-33
Module 21.11

CP, IQGAP1, LY6E, TAPBP, IL33, S100A11, CD14, CTPS, LIMS1,
FAM81A, MSR1, DPYS, MOB1A, RAP1B, CDKL1, TLR2, CERS5,
TTC41, SLC2A4, ACSM3, CISH, JUN, TNS3, EHD4, TMEM47,
KCNC4, MFSD4, LRRC14B,

TABLE 27-34
Module 21.111

LY6C2, HEBP1, ANGPT2, ITPR2, LIX1L, BNIP2, F2R, MT2, ACAD12, LY6C1, ARPC5,
RAPH1, SLC4A1, ABCA9, NOTUM, PRKX, FITM1, TMOD3, FKBP7, CACNA2D1,
SLC44A2, DPYSL2, IRGM2, WHRN, ITPRIPL2, MAGIX, S1PR3, GCGR, ISOC2B,
DIAPH2, SAV1, ARXES2, SFXN4, FNDC3B, 1110034G24RIK, 3010026O09RIK,
PPP3CC, METTL21B, GM10272, LIPO1,

TABLE 27-35
Module 21.112

SLC19A3, LY6A, B4GALNT1, IFIT2, CTH, DPYSL3, AHCY, FRK,
SLC22A15, ALPK1, IL7R, GGACT, DAPP1, CYP2D22,
1500009L16RIK, PHYHIPL, AGRN, PCK1, NEU1, PLPPR1,
MRGPRH, GM4737, GM13212, EIF4E3, DSEL, BEND7, IL15, RILPL2,

TABLE 27-36
Module 21.113

ANXA3, VAT1, LITAF, SMURF1, SERPINB6B, AKT3, PDZKIIP1, TIMP2, CPNE8,
IFNGR1, CD47, TLR7, TTC39B, RGS5, DDAH2, KLF7, HMGA1-RS1, ST3GAL1,
CACNB4, GM2115, CD2AP, ABCA1, PROS1, SLC11A2, FAT4, UGCG, PTPN1, TFEC,
BOC, CHCHD5, OLFR54, TMEM25, KRAS, MAP4K4, PFKM, CREB5, ZC2HCIA,
STIM2, SOAT1, CCBL1, NELL2,

TABLE 27-37
Module 21.114

SPP1, TNFRSF12A, CFTR, ITGB1, B4GALT1, RHOB, RHOC, ABCA4, MSN, EIF6,
CYP2J13, HSPB1, 1700016C15RIK, GREM1, CAR15, CYFIP2, CREB3, ADAMTSL2,
NDN, ANO10, INMT, STOM, PDE4C, RHOD, LARGE, ATP1B3, FAM171B, TMIGD1,
OLFR1416, CSRP1, CLCA3B, FRS3, CYB5R3, IGSF5, NPR2, GDF15, GM128,
6720489N17RIK,

TABLE 27-38
Module 22.117

OGDH, ACSM2, ASB13, MDH2, PLS1, MLEC, CCL19, LACE1, MRPS7, MAGT1,
ENPEP, SDHD, PRDX3, NDUFS1, CACNA1D, SLC5A12, KIDINS220, ECHDC1, LIPA,
GALNT14, SFXN3, BSND, TCIRG1, SLC25A11, MFSD6, ST8SIA1, CASP12, CLRN3,
HADHA, ACSS3, SDHA, HHATL, TBC1D9, MRPL50, DMXL1, ENTPD5, NIPAL1,
HSPA8, ABCC3, IL18R1, VDAC3, GADD45B, CHD3, KCNK1, UFM1, OPHN1,
TMEM140, ERC2, LRPPRC, 9030617O03RIK, LRRC3, HMGCS1, PLAG1, ITK, PTCD2,
MUC20, AP5M1, AIFM1, CACYBP, SH3PXD2B, KDM7A, HOXB8, LAMA3, ORMDL2,
RNF152, MYO15B, KIT, ZFP626, EVC, SLC6A18, KCTD21, SOX9, ST3GAL5, GM4952,
NUDT7, C6, RPS27RT, NUFIP1, USP27X, NSG1, CDX1, COPS8, CCSER2, ADSL, LDHB,
ARHGAP24, SEMA3B, SULF1, MAML2, KDM1B, WDFY3, MAPK13, BAIAP2L2,
AU019823, GATA6, ACOT6, HIBCH, RNF186, RHBDF2, ARHGAP31, TRP53I11,
GM684, CTXN3, OLFR558, GM6377, IFT43, MGAT5, CAV3,

TABLE 27-39
Module 23.12

NUSAP1, MKI67, TOP2A, RBL1, TPX2, PLK1, KNSTRN, STMN1,
SMC2, MAD2L1, PRR11, PBK, ECT2, TACC3, KIF11, RRM2,
CCNB2, KCNMB1, UBE2C, CD34, CDKN3, PRC1, CYP3A13, ZIM1,
DEPDC1A, CDK1, NCAPH, CDC20, BIRC5, HIST1H3E, PODXL,
ZBTB3, AP1M2,

TABLE 27-40
Module 24.122

S100A6, BGN, THBS2, PDGFRB, SERPING1, MMP2, CXCL16, IFNGR2, GVIN1, NEXN,
LRP1, DDR2, CALD1, LAMA2, EDNRA, CPXM1, IL6ST, MXRA8, FAM13A, FHL2,
MAN1C1, DCN, RNF122, ANTXR1, IGSF10, MOB3A, LXN, PLXNA1, PDLIM1, P3H4,
RBMS3, ZEB2, GUCY1B3, LBH, NEK6, NRP2, TSHZ2, PRELID2, FAM175A, FAM102B,
KCNK6,

TABLE 27-41
Module 24.123

FN1, GBP9, COL15A1, THBS1, LOXL1, FBN1, TNC, NID1, DSE, EMP3, VCL, FSTL1,
GBP10, VIM, DCLK1, COL8A1, FREM1, RCN3, COL1A2, SERPINA1B, PSD3, SPARC,
GBP4, CDH11, ANKRD1, SRPX2, CNN2, STAT1, ANK, CD93, SCGB1B7, IRF9, MRC2,
MCM5, RNF213, GBP8, ADAM19, NGEF, COL3A1, PPIL6, TWF1, PXDN, COL1A1,
IL1R1, 6-MAR, FNIP2, PEAK1, ESRRB, MCM4, PPARGC1A, ECSCR, CRYAB, GRB7,
CFL1, TDRD3, DES, NCEH1, VMN1R27, CPNE2, WFDC16, PTPN23, ASCC3, HSD11B1,
NOP58, WNK4, 1-MAR, AKR1C13, KLF15, TOX3, IFIT1BL2, SH3D19, OLFR1342, 11-
SEP, GLUL, KLF10, INSIG1, PEMT, GSTO2, MORN4, CIRH1A, CDKL2, CKS1B,
EMILIN1, CKLF, SCGB1B29, BNIP3, GUCY1A2, GSTT3, HEYL, ETS1, GOT1, RAB27A,
ZKSCAN7, ENDOU, 2-SEP, LIN52, CEP192, SEMA3D, IER5L, ATXN1L, RBBP8,
CDC42EP3, ADORA2B, CCRL2, ISLR, RABL3,

TABLE 27-42
Module 24.124

FRZB, TRPM6, SVEP1, HGF, MCM6, NAT14, SYN2, SERPINA1C, ITGA9, PLOD2,
ERAP1, VGLL3, H2AFX, EFHD1, ALDH1A2, ASB15, CML2, SPRR1A, CMPK2, SUGT1,
ATP12A, 4930503L19RIK, GBA, EMCN, CCNB1IP1, B4GALT5, FIGN, ANLN, OLFR324,
MCM2, LRRC8C, HIST1H2AO, PSMG4, SLFN1,

TABLE 27-43
Module 24.126

FBLN5, OLFR1393, COL14A1, NT5DC2, HPGD, KCNS1,
A930018P22RIK, SLC40A1, OLFR320, 4930563D23RIK,
ITGA8, NID2, GDF10, FGFBP1, OLFR1451, SLC35G2,
HUNK, PPP2R3D, SERPINH1, ITIH5,

TABLE 27-44
Module 25.127

ST13, ANKRD13C, GK, DOPEY2, OPA1, ATP6V1A, APMAP,
SLC22A5, ETFDH, ITPR1, NXT2, GRAMD1A, SLC16A14,
A230050P20RIK, MEX3A, TNFSF10, HOXC8, TMEM229A,
TANC2, TESK2, VDAC1, HSD17B4, FUT8, FBXO22,

TABLE 27-45
Module 26.13

SPARCL1, MAP3K8, IRAK3, OSMR, MMP14, HIP1, LBP, STAT3, ART4, MEF2A,
SPON1, SERPINF1, LRRC66, MMP23, CPED1, P2RX7, ARRDC4, HAUS4, RASA3,
SLC7A1, GSTA3, ALCAM, SLC36A4, CXCL13, BIRC3, RDH7, GDPD3, ZFP759, CD55,
SWAP70, PLEKHG1, TNFRSF10B, PON2, CYGB, CLDN6, PLK2, CHST1, OLFR168,
SOX18, CYP2J8, SEMA4C, AVPR1A, ISG20L2, LONP1, FMNL2, CENPC1, AGL, BOK,
EPB41L2, NFATC1, AGPAT4, FXYD1, P2RY12, MTSS1, PECAM1, ZFP677, IZUMO4,

TABLE 27-46
Module 27.132

RNASE6, IL2RB, SLAMF7, CD3E, CD5, WDFY4, DENND1C, CD3G,
CLEC9A, HEXA, TSPAN32, CD226, TNS1, IGF1, PLCB2, LIMS2,
FLT3L, SELL, 3-SEP, FGL2, LONRF3, FBLN1, BC052040, CD96,
MCEMP1, ZFP938, GM17455,

TABLE 27-47
Module 29.139

EXOC5, DDX3X, GNAI3, PPP1CB, SUPT16, CAPZA2, LGALSL, MAT2A, ABCD3,
BMPR1A, TMEM106B, TXNRD1, ADSS, USP47, MATR3, IPO7, ZFP433, RBM45, TMF1,
USP24, PLEKHA1, APOL9B, ZFP873, IWS1, SLC30A5, RASA1, D15ERTD621E, RHOA,
KCNJ16, STRN3, HIF1AN, ZFP933, DHX9, ATP13A3, HSDL2, BTAF1, 1700029J07RIK,
CALU, PSAT1, NUDT2, SIRT5, SRSF5, STK38L, COMMD5, SLC46A3, MPP1, CHMP5,
YPEL3, ZFP867, LMO1, FRMD4B, PNN, TNFRSF21, DDX28, DEPDC1B, CCNYL1,
SGK1, TARM1, DDX10, NCKAP1, NAMPT, EIF2S1, CLDN19, S100A8, SAMD12,
S100A9, MAP3K5, ADAM4, AI467606, DUSP23, RAB21, ORAI3, NFIL3, GM5072,
GM13769, OLFR566, OLFR47, CLEC14A, PPP1R3D, FCRL5, IL1F9, TMEM41A,
SMARCAD1, ATP11B, RAD21, ATL3, PNP2, ATE1, 2010315B03RIK, CAND2, APOC3,
DDB1, CYP2J6, BCL2L1, SLC19A1, REST, PACRG, LCE3B, CAPZA1, LYPD5, PFPL,
TRIM68, INSM1, CPM, SWSAP1,

TABLE 27-48
Module 29.14

7-SEP, NARS, A230046K03RIK, PIGR, RRM1, CYP2A4, ICAM4,
TMEM184C, ZFP72, COL4A2, ZDHHC13, DEFB23, EPO, KRTAP11-1,
BRF2, RARRES2, HSPB3, UBXN4, DPPA5A, FGFBP3, WDR5B,

Table 28: Gene expression signatures to separate LN disease stages
The genes in each of Tables 28-1 to 28-22 can be used as effective biomarkers for classifying the LN disease state of the patient.

TABLE 28-1
Amino Acid Metabolism

AASS, AGXT, AGXT2, ALDH4A1, ALDH7A1, ARG1, GLUD1, GOT1,
GPT2, GRHPR, IVD, MCCC2, OAT, OTC, OXCT1, PC, PSAT1, SDS

TABLE 28-2
Antigen Presenting Cell

CD1D, CD74, CIITA, CLEC10A, HLA-DMA, HLA-DMB, HLA-DOA,
HLA-DOB, HLA-DQA1, HLA-DQB1, HLA-DRA, HLA-DRB5, RFX5,
SCIMP,

TABLE 28-3
CD8 T cell

CD8A, CD8B

TABLE 28-4
Fatty Acid Oxidation

ABCD1, ABCD2, ACAA2, ACACB, ACAD11, ACADL, ACADM, ACADVL, ACAT1,
ACAT2, ACOX1, ACOX2, ACOXL, ACSBG2, ADIPOQ, AKT2, AUH, BDH2, CPT1A,
CPT2, DECR1, ECHDC2, ECHS1, ECI2, EHHADH, ETFB, ETFDH, FABP1, GCDH,
HAAO, HACL1, HADH, HADHA, HADHB, HIBCH, HSD17B4, IRS1, IVD, LEP,
MLYCD, PEX13, PEX2, PEX5, PEX7, PLIN5, PPARD, PPARG, PPARGCIA, SESN2,
SLC27A2, TWIST1

TABLE 28-5
GC B Cell

FCRLB, GCSAM, KLHL6, MYBL1, NUGGC, RGS13

TABLE 28-6
General Mitochondria

ACAD10, ACAD8, ACADSB, ACP6, ACSM2A, ADHFE1, AGK, AGMAT, AGXT2,
ALDH5A1, AMT, ARMCX1, ARMCX2, ARMCX3, BCKDHB, BCO2, CLPB, CLYBL,
CMC1, COA5, COQ2, COQ4, COQ7, CYB5B, CYP11A1, CYP27A1, CYP27B1, CYP2U1,
DDAH1, DHODH, DHTKD1, DMGDH, EFHD1, ETHE1, FAHD1, FAHD2A, FAHD2B,
FAM72A, FASTK, GAD1, GK, GLS2, GLYAT, GPAT2, GSTK1, HEMK1, HINT2,
HOGA1, IBA57, IMMP2L, IVD, KMO, LDHD, LIPT1, MACROD1, MAOA, MCCC2,
MCEE, MCUB, ME3, METTL15, MICU3, MIX23, MPST, MRS2, MTHFD2L, NAXE,
NME4, NMNAT3, PC, PCCA, PCCB, PDE12, PGS1, PLEKHN1, PLSCR3, PPOX, PPTC7,
PRODH, PRODH2, PYCR1, REXO2, SFXN2,

TABLE 28-7
IG Chains

IGH, IGHA1, IGHA2, IGHD2-15, IGHD2-21, IGHD3-10, IGHD3-16, IGHD3-3, IGHE,
IGHG1, IGHG2, IGHG3, IGHG4, IGHGP, IGHJ1, IGHJ2, IGHJ3, IGHJ4, IGHJ5, IGHJ6,
IGHMBP2, IGHV1-18, IGHV1-2, IGHV1-24, IGHV1-3, IGHV1-45, IGHV1-46, IGHV1-58,
IGHV1-69-2, IGHV2-26, IGHV2-5, IGHV2-70, IGHV3-11, IGHV3-13, IGHV3-15, IGHV3-
16, IGHV3-20, IGHV3-21, IGHV3-23, IGHV3-33, IGHV3-35, IGHV3-38, IGHV3-43,
IGHV3-47, IGHV3-48, IGHV3-49, IGHV3-53, IGHV3-54, IGHV3-7, IGHV3-72, IGHV3-
73, IGHV3-74, IGHV3OR16-8, IGHV4-28, IGHV4-30-2, IGHV4-31, IGHV4-34, IGHV4-
39, IGHV4-59, IGHV4-61, IGHV5-51, IGHV5-78, IGHV6-1, IGHV7-34-1, IGHV7-81, IGK,
IGKC, IGKJ1, IGKJ2, IGKJ3, IGKJ4, IGKJ5, IGKV@, IGKV1-16, IGKV1-17, IGKV1-27,
IGKV1-5, IGKV1-6, IGKV1-9, IGKV1D-16, IGKV1D-17, IGKV1D-27, IGKV1D-43,
IGKV1D-8, IGKV2D-24, IGKV2D-26, IGKV3-20, IGKV3-7, IGKV3D-20, IGKV3D-7,
IGKV4-1, IGKV5-2, IGL, IGLC1, IGLC2, IGLC6, IGLC7, IGLJ3, IGLJ6, IGLL1, IGLL3P,
IGLL5, IGLV@, IGLV1-36, IGLV1-40, IGLV1-44, IGLV1-47, IGLV1-50, IGLV10-54,
IGLV2-11, IGLV2-14, IGLV2-18, IGLV2-23, IGLV2-5, IGLV2-8, IGLV3-1, IGLV3-10,
IGLV3-12, IGLV3-16, IGLV3-19, IGLV3-21, IGLV3-22, IGLV3-25, IGLV3-27, IGLV3-9,
IGLV4-3, IGLV4-60, IGLV4-69, IGLV5-37, IGLV5-45, IGLV5-48, IGLV6-57, IGLV7-43,
IGLV7-46, IGLV8-61, IGLV9-49, IGLVI-56, IGLVI-70,

TABLE 28-8
Kidney Cell

ACOT4, ACSM2A, AGXT2, ALDH4A1, BBOX1, CD24, CDH16, CHST9, CLDN10,
CLDN16, CLDN2, CMBL, COL4A3, COL4A4, COL4A5, CTXN3, CXCL14, CYP27B1,
CYP4A22, DAO, DCDC2, DMGDH, DNM3, EPCAM, ESM1, ETNK2, FAM151A, G6PC1,
GALNT14, GLYAT, GNE, HEY1, HNF1B, HOGA1, HOXD3, HPD, HS3ST1, HSPA12A,
HYKK, ITGB8, KIF12, LAMB2, NAT8, NR1H4, P3H2, PANK1, PAX8, PCK1, PIPOX,
PKHD1, PLLP, PRODH, PRODH2, PROM1, PROZ, RAB11FIP3, RASIP1, REN, RHBG,
RHCG, RNF152, RNF183, SALL1, SFXN2, SGK2, SHISA3, SIM2, SLC13A3, SLC16A4,
SLC17A1, SLC17A3, SLC22A6, SLC22A7, SLC30A2, SLC34A3, SLC3A1, SLC7A13,
SLC7A7, SOD3, SOSTDC1, SULT1C2, TMEM174, TMEM207, TMPRSS4, TRABD2B,
UGT1A4, UGT1A9, UGT2B28, UGT2B28, UGT2B28, UGT3A1, UGT3A1, UGT3A2,
UGT8, UPK1B, WNK4,

TABLE 28-9
Kidney Distal Tubule

ATP6V0D2, ATP6V1G3, CALB1, CDH1, FXYD2, KCNJ1, KCNJ16,
PPP1R1A, PTH1R, PVALB, SLC12A1, SLC12A3, TMEM72,
UMOD, WFDC2,

TABLE 28-10
Kidney Loop of Henle

CLCNKA, CLCNKB, CLCNKB, KNG1

TABLE 28-11
Kidney Prox Convoluted Tubule

ACY1, AQP1, ASS1, CLTRN, CRYAA, CRYAB, CUBN, DDAH1,
FMO1, GPX3, HNF1A, MIOX, NOX4, PAH, PAX2, PDZK1,
PDZK1IP1, SLC13A1, SLC22A12, SLC22A13, SLC22A2,
SLC22A8, SLC34A1, SLC36A2, SLC4A4, SLC5A2, SLC6A18,

TABLE 28-12
LDG

CTSG, ELANE, LCN2, MPO, OSM

TABLE 28-13
Monocyte-Macrophage

ACE, ADAM8, ADGRE1, APOBR, APOC1, C1QA, C1QB, C1QC,
C2, C4B, C6, CCL7, CCL8, CD14, CD163, CD300E, CD5L,
CFB, CFD, CFP, CSF1R, CXCL13, FOLR2, IFI16, IL31RA,
LGALS12, LYVE1, MARCO, MEGF10, MERTK, MFGE8, MRC1,
MS4A4A, MSR1, PLA2G5, SCARF2, SIGLEC1,

TABLE 28-14
Myeloid Cell

AIF1, AOAH, ARG1, BATF2, BMX, BPI, CD300LF, CD33,
CD68, CLEC12A, CLEC4A, CLEC4C, CLEC4D, CLEC4E,
CLEC4G, CLEC5A, CLEC6A, CLEC7A, CSF2RA, CSF2RB,
CSF3R, CST3, CXCL9, CYBA, CYBB, FCER1A, FCGR1A,
FCGR2A, FCGR3A, FFAR2, FGR, IGSF6, IL1RN, LILRB4,
LYZ, MPEG1, MS4A6A, NLRP12, NLRP3, OLR1, P2RX5,
PILRA, PLA2G2D, S100A9, SCARB1, SERPING1,
SLAMF8, TREM1, TREML4, VSIG4,

TABLE 28-15
NK Cell

KLRB1, KLRB1, NCR1, SERPINB9, STYK1

TABLE 28-16
Oxidative Phosphorylation

ATP5F1A, ATP5F1B, ATP5F1D, ATP5MC1, ATP5MC2, ATP5MC3,
ATP5MF, ATP5PD, ATP5PF, ATP5PO, BCS1L, CEP89, COA3,
COA4, COA5, COA7, COX10, COX11, COX14, COX15, COX16,
COX17, COX18, COX19, COX20, COX4I1, COX4I2, COX5A,
COX6A1, COX6B1, COX7A1, COX7A2, COX7A2L, COX7B,
COX8A, CYC1, CYCS, NDUFA1, NDUFA10, NDUFA11, NDUFA12,
NDUFA13, NDUFA2, NDUFA3, NDUFA7, NDUFA8, NDUFA9,
NDUFAB1, NDUFAF1, NDUFAF2, NDUFAF3, NDUFAF4,
NDUFAF5, NDUFAF6, NDUFAF7, NDUFB10, NDUFB11, NDUFB2,
NDUFB3, NDUFB4, NDUFB5, NDUFB6, NDUFB7, NDUFB8,
NDUFB9, NDUFC1, NDUFC2, NDUFS1, NDUFS2, NDUFS3,
NDUFS4, NDUFS6, NDUFS7, NDUFS8, NDUFV1, NDUFV2,
NDUFV3, NUBPL, OXA1L, SCO2, SLC25A4, TIMMDC1,
TMEM126B, TRAP1, UQCC1, UQCC2, UQCR10, UQCR11,
UQCRB, UQCRC1, UQCRC2, UQCRFS1, UQCRH, UQCRQ,

TABLE 28-17
pDC

BST2, CD300C, CD33, TCF4

TABLE 28-18
Platelet

CD9, CXCL6, ESAM, GP1BA, GP5, GP6, GP9, LY6G6D, MMRN1,
MYL9, PEAR1, PF4, PLEK, PPBP, SLC35D3, TREML1, TSPAN9,

TABLE 28-19
Podocyte

NPHS1, NPHS2, PODXL, SYNPO, WT1

TABLE 28-20
TCA cycle

ACO2, CS, DLAT, DLD, DLST, FH, GLUD1, IDH1, IDH2, IDH3A,
IDH3B, IDH3G, MDH2, MPC1, MPC2, OGDH, OGDHL, PDHA1,
PDHB, PDHX, PDK1, PDK2, PDK3, PDP2, PDPR, SDHA, SDHAF2,
SDHB, SDHC, SDHD, SUCLA2, SUCLG1, SUCLG2, SUGCT,

TABLE 28-21
Tfh Cell

ASCL2, BCL6, CXCR5, ICOS, NT5E, PDCD1, TNFRSF4,

TABLE 28-22
Th17 Cell

AHR, CD3D, CD3E, CD3G, IL17A, IL17F, IL21, IL22, IRF4, NFXL1,
PGF, RORA, RORC, TRAF6,

### REFERENCES

1. Davidson A. What is damaging the kidney in lupus nephritis?. Nat. Rev. Rheumatol. 2016; 12(3):143-153.
2. Maria N I, Davidson A. Protecting the kidney in systemic lupus erythematosus: from diagnosis to therapy. Nat. Rev. Rheumatol. 2020; 16(5):255-267.
3. Mimura I, Nangaku M. The suffocating kidney: Tubulointerstitial hypoxia in end-stage renal disease. Nat. Rev. Nephrol. 2010; 6(11):667-678.
4. Suárez-Fueyo A, et al. T cells and autoimmune kidney disease. Nat. Rev. Nephrol. 2017; 13(6):329-343.
5. Nangaku M. Chronic hypoxia and tubulointerstitial injury: a final common pathway to end-stage renal failure. J. Am. Soc. Nephrol. 2006; 17(1):17-25.
6. Leatherwood C, et al. Clinical characteristics and renal prognosis associated with interstitial fibrosis and tubular atrophy (IFTA) and vascular injury in lupus nephritis biopsies. Semin. Arthritis Rheum. 2019; 49(3):396-404.
7. Liu B-C, et al. Renal tubule injury: a driving force toward chronic kidney disease. Kidney Int. 2018; 93(3):568-579.
8. Weening J J, et al. The Classification of Glomerulonephritis in Systemic Lupus Erythematosus Revisited. J. Am. Soc. Nephrol. 2004; 15(2):241-250.
9. Ortega L M, et al. Lupus nephritis: Pathologic features, epidemiology and a guide to therapeutic decisions. Lupus 2010; 19(5):557-574.
10. Hsieh C, et al. Predicting outcomes of lupus nephritis with tubulointerstitial inflammation and scarring. Arthritis Care Res. (Hoboken). 2011; 63(6):865-874.
11. Schwartz M M, et al. Irreproducibility of the activity and chronicity indices limits their utility in the management of lupus nephritis. Lupus Nephritis Collaborative Study Group. Am. J. kidney Dis. Off J. Natl. Kidney Found 1993; 21(4):374-377.
12. Mubarak M, Nasri H. ISN/RPS 2003 classification of lupus nephritis: time to take a look on the achievements and limitations of the schema. J. Nephropathol. 2014; 3(3):87-90.
13. Almaani S, et al. Rethinking Lupus Nephritis Classification on a Molecular Level. J. Clin. Med 2019; 8(10). doi:10.3390/jcm8101524
14. Waters S T, et al. NZM2328: a new mouse model of systemic lupus erythematosus with unique genetic susceptibility loci. Clin. Immunol. 2001; 100(3):372-383.
15. Waters S T, et al. Breaking Tolerance to Double Stranded DNA, Nucleosome, and Other Nuclear Antigens Is Not Required for the Pathogenesis of Lupus Glomerulonephritis. J. Exp. Med 2004; 199(2):255-264.
16. Ge Y, et al. Cgnz1 allele confers kidney resistance to damage preventing progression of immune complex-mediated acute lupus glomerulonephritis. J. Exp. Med 2013; 210(11):2387-2401.
17. Bagavant H, et al. Role for Nephritogenic T Cells in Lupus Glomerulonephritis: Progression to Renal Failure Is Accompanied by T Cell Activation and Expansion in Regional Lymph Nodes. J. Immunol. 2006; 177(11):8258-8265.
18. Fu S M, et al. Pathogenesis of proliferative lupus nephritis from a historical and personal perspective. Clin. Immunol. 2017; 185:51-58.
19. Peterson K S, et al. Characterization of heterogeneity in the molecular pathogenesis of lupus nephritis from transcriptional profiles of laser-captured glomeruli. J. Clin. Invest. 2004; 113(12):1722-1733.
20. Hanzelmann S, Castelo R, Guinney J. GSVA: gene set variation analysis for microarray and RNA-Seq data. BMC Bioinformatics 2013; 14(1):7.
21. Schwartzman-Morris J, Putterman C. Gender Differences in the Pathogenesis and Outcome of Lupus and of Lupus Nephritis. Clin. Dev. Immunol. 2012; 2012:604892.
22. Lahita R G. The role of sex hormones in systemic lupus erythematosus. Curr. Opin. Rheumatol. 1999; 11(5):352-356.
23. Moulton V R. Sex Hormones in Acquired Immunity and Autoimmune Disease. Front. Immunol. 2018; 9. doi:10.3389/fimmu.2018.02279
24. Kramer A, et al. Causal analysis approaches in Ingenuity Pathway Analysis. Bioinformatics 2014; 30(4):523-530.
25. Mannik M, et al. Multiple autoantibodies form the glomerular immune deposits in patients with systemic lupus erythematosus. J. Rheumatol. 2003; 30(7):1495-1504.
26. Reynolds J, et al. Anti-CD8 monoclonal antibody therapy is effective in the prevention and treatment of experimental autoimmune glomerulonephritis. J. Am. Soc. Nephrol. 2002; 13(2):359-369.
27. Chen A, et al. Role of CD8+ T cells in crescentic glomerulonephritis. Nephrol. Dial. Transplant. Off Publ. Eur. Dial. Transpl. Assoc.-Eur. Ren. Assoc. 2020; 35(4):564-572.
28. Couzi L, et al. Predominance of CD8+T lymphocytes among periglomerular infiltrating cells and link to the prognosis of class III and class IV lupus nephritis. Arthritis Rheum. 2007; 56(7):2362-2370.
29. Kim H J, et al. CD8+ T regulatory cells express the Ly49 class I MHC receptor and are defective in autoimmune prone B6-Yaa mice. Proc. Natl. Acad. Sci. U.S.A 2011; 108(5):2010-2015.
30. Arazi A, et al. The immune cell landscape in kidneys of patients with lupus nephritis. Nat. Immunol. 2019; 20(7):902-914.
31. Toro-Dominguez D, et al. Stratification of Systemic Lupus Erythematosus Patients Into Three Groups of Disease Activity Progression According to Longitudinal Gene Expression. Arthritis Rheumatol. 2018; 70(12):2025-2035.
32. Der E, et al. Tubular cell and keratinocyte single-cell transcriptomics applied to lupus nephritis reveal type I IFN and fibrosis relevant pathways. Nat. Immunol. 2019; 20(7):915-927.
33. Sung S J, et al. Dependence of Glomerulonephritis Induction on Novel Intraglomerular Alternatively Activated Bone Marrow-Derived Macrophages and Mac-1 and PD-L1 in Lupus-Prone NZM2328 Mice. J. Immunol. 2017; 198(7):2589-2601.
34. Sung S-S J, Fu S M. Interactions among glomerulus infiltrating macrophages and intrinsic cells via cytokines in chronic lupus glomerulonephritis. J. Autoimmun. 2020; 106:102331.
35. Kuriakose J, et al. Patrolling monocytes promote the pathogenesis of early lupus-like glomerulonephritis. J. Clin. Invest. 2019; 129(6):2251-2265.
36. Schiffer L, et al. Activated renal macrophages are markers of disease onset and disease remission in lupus nephritis. J. Immunol. 2008; 180(3):1938-1947.
37. Ma R, et al. Intrarenal macrophage infiltration induced by T cells is associated with podocyte injury in lupus nephritis patients. Lupus 2016; 25(14):1577-1586.
38. Sakhi H, et al. Podocyte Injury in Lupus Nephritis. J. Clin. Med. 2019; 8(9). doi:10.3390/jcm8091340
39. Tian Y, et al. Nestin protects podocyte from injury in lupus nephritis by mitophagy and oxidative stress. Cell Death Dis. 2020; 11(5):319.
40. Deng W, et al. Hypoxia inducible factor-1 alpha promotes mesangial cell proliferation in lupus nephritis. Am. J. Nephrol. 2014; 40(6):507-515.
41. Chen P M, et al. Kidney tissue hypoxia dictates T cell-mediated injury in murine lupus nephritis. Sci. Transl. Med. 2020; 12(538). doi:10.1126/scitranslmed.aay1620
42. Fine L G, Bandyopadhay D, Norman J T. Is there a common mechanism for the progression of different types of renal diseases other than proteinuria?Towards the unifying theme of chronic hypoxia. Kidney Int. Suppl. 2000; 75:S22-6.
43. Fortner K A, et al. Targeting mitochondrial oxidative stress with MitoQ reduces NET formation and kidney disease in lupus-prone MRL-lpr mice. Lupus Sci. Med. 2020; 7(1). doi:10.1136/lupus-2020-000387
44. Hong S, Healy H, Kassianos A J. The Emerging Role of Renal Tubular Epithelial Cells in the Immunological Pathophysiology of Lupus Nephritis. Front. Immunol. 2020; 11(September): 1-8.
45. Bonventre J V. Kidney injury molecule-1 (KIM-1): A urinary biomarker and much more. Nephrol. Dial. Transplant. 2009; 24(11):3265-3268.
46. Zhou Y, et al. Comparison of kidney injury molecule-1 and other nephrotoxicity biomarkers in urine and kidney following acute exposure to gentamicin, mercury, and chromium. Toxicol. Sci. 2008; 101(1):159-170.
47. Castillo-Rodriguez E, et al. Kidney Injury Marker 1 and Neutrophil Gelatinase-Associated Lipocalin in Chronic Kidney Disease. Nephron 2017; 136(4):263-267.
48. Moschen A R, et al. Lipocalin-2: A Master Mediator of Intestinal and Metabolic Inflammation. Trends Endocrinol. Metab. 2017; 28(5):388-397.
49. Ansar Ahmed S, Penhale W J, Talal N. Sex hormones, immune responses, and autoimmune diseases. Mechanisms of sex hormone action. Am. J. Pathol. 1985; 121(3):531-551.
50. Cutolo M, Wilder R L. Different roles for androgens and estrogens in the susceptibility to autoimmune rheumatic diseases. Rheum. Dis. Clin. North Am. 2000; 26(4):825-839.
51. Shim G-J, et al. Autoimmune glomerulonephritis with spontaneous formation of splenic germinal centers in mice lacking the estrogen receptor alpha gene. Proc. Natl. Acad. Sci. 2004; 101(6):1720-1724.
52. Rider V, et al. Molecular mechanisms involved in the estrogen-dependent regulation of calcineurin in systemic lupus erythematosus T cells. Clin. Immunol. 2000; 95(2):124-134.
53. Lang T J, et al. Increased severity of murine lupus in female mice is due to enhanced expansion of pathogenic T cells. J. Immunol. 2003; 171(11):5795-5801.
54. Graham J H, Yoachim S D, Gould K A. Estrogen Receptor Alpha Signaling Is Responsible for the Female Sex Bias in the Loss of Tolerance and Immune Cell Activation Induced by the Lupus Susceptibility Locus Sleib. Front. Immunol. 2020; 11. doi:10.3389/fimmu.2020.582214
55. Cutolo M, et al. Androgens and estrogens modulate the immune and inflammatory responses in rheumatoid arthritis. Ann. N. Y. Acad. Sci. 2002; 966:131-142.
56. Gubbels Bupp M R, Jorgensen T N. Androgen-Induced Immunosuppression. Front. Immunol. 2018; 9:794.
57. Aitman T J, et al. Copy number polymorphism in Fcgr3 predisposes to glomerulonephritis in rats and humans. Nature 2006; 439(7078):851-855.
58. Stratigou V, et al. Altered expression of signalling lymphocyte activation molecule receptors in T-cells from lupus nephritis patients-A potential biomarker of disease activity. Rheumatol. (United Kingdom) 2017; 56(7):1206-1216.
59. Zhou D, et al. Wnt/β-catenin signaling in kidney injury and repair: A double-edged sword. Lab. Investig. 2016; 96(2):156-167.
60. Bult C J, et al. Mouse Genome Database (MGD) 2019. Nucleic Acids Res. 2019; 47(D1):D801-D806.
61. Heng T S P, et al. The Immunological Genome Project: networks of gene expression in immune cells. Nat. Immunol. 2008; 9(10):1091-1094.
62. Kingsmore K M, et al. Altered expression of genes controlling metabolism characterizes the tissue response to immune injury in lupus. Sci. Rep. 2021; 11(1):14789.

### Example 4: Transcriptomic Analysis of Lupus Nephritis Kidneys Identifies Molecular Endotypes

Predicting the course or response to treatment of lupus nephritis (LN) from standard renal biopsies is problematic. We analyzed bulk RNA from renal biopsies and paired blood to understand and determine gene expression based lupus nephritis and molecular biomarkers of LN.

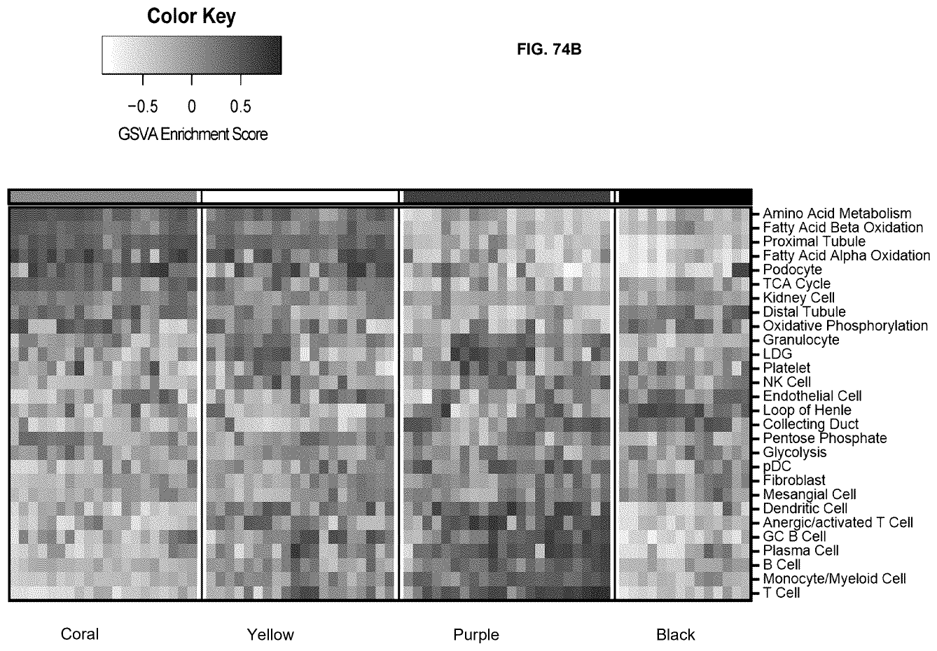

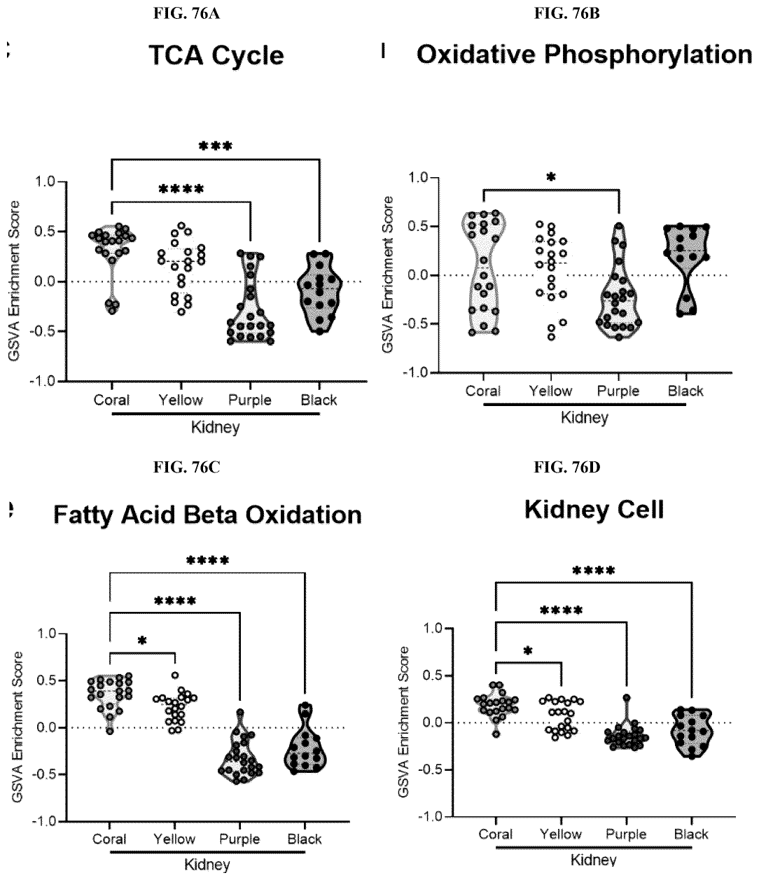

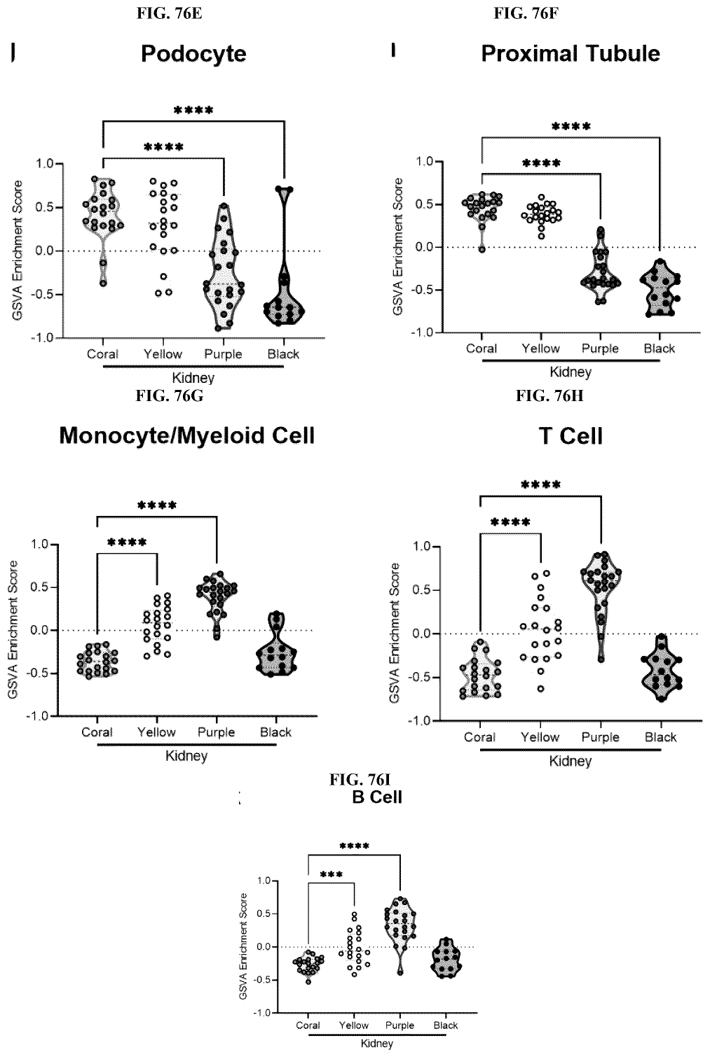

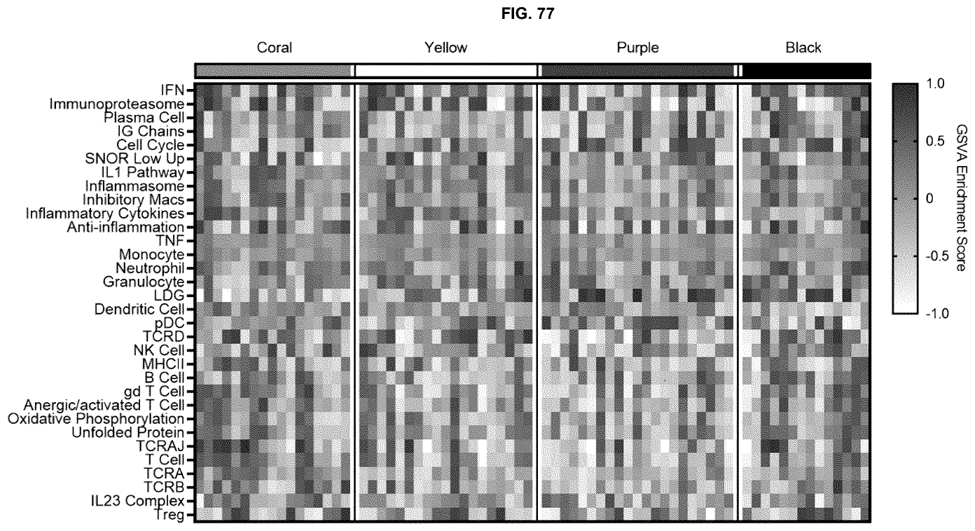

Methods: Gene expression data was collected from the whole kidney and blood of patients with LN and additional datasets were identified in the Gene Expression Omnibus (GEO) (Table 24). Enrichment of informative modules of co-expressed genes in individual samples was analyzed using Gene Set Variation Analysis (GSVA). Gene modules identifying immune/inflammatory cells, resident kidney cells, and metabolic and inflammatory processes as described (1) were employed where appropriate. Kidney biopsy histology and immunofluorescence were scored by a blinded clinical pathologist. Gene sets used as input for GSVA for FIGS. 74 and 76 are listed in Tables 23-1 to 23-28. For example, Table 23-1 lists Amino Acid Metabolism signature genes. Gene sets used as input for GSVA for FIGS. 77, 78 and 80 are listed in Tables 25-1 to 25-32. The genes are grouped according to cell type or pathway signature.

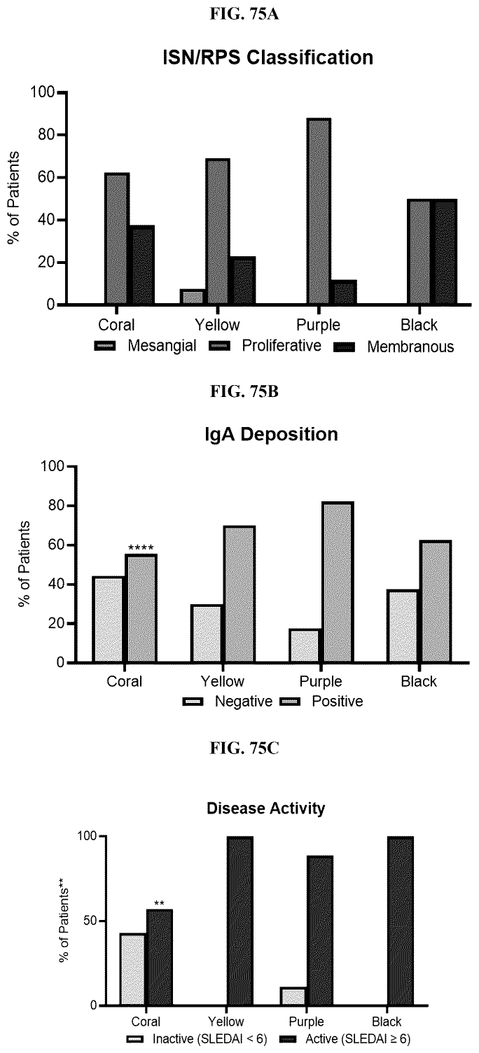

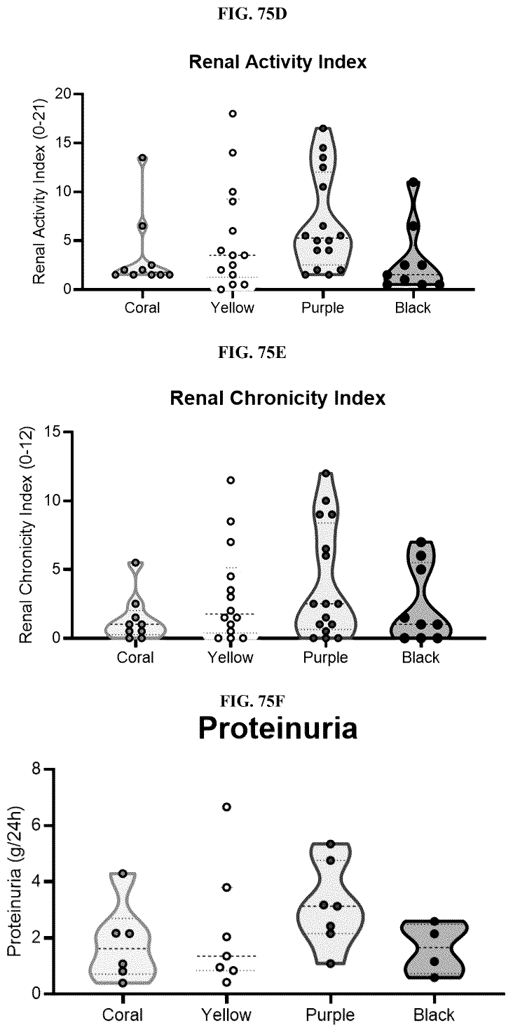

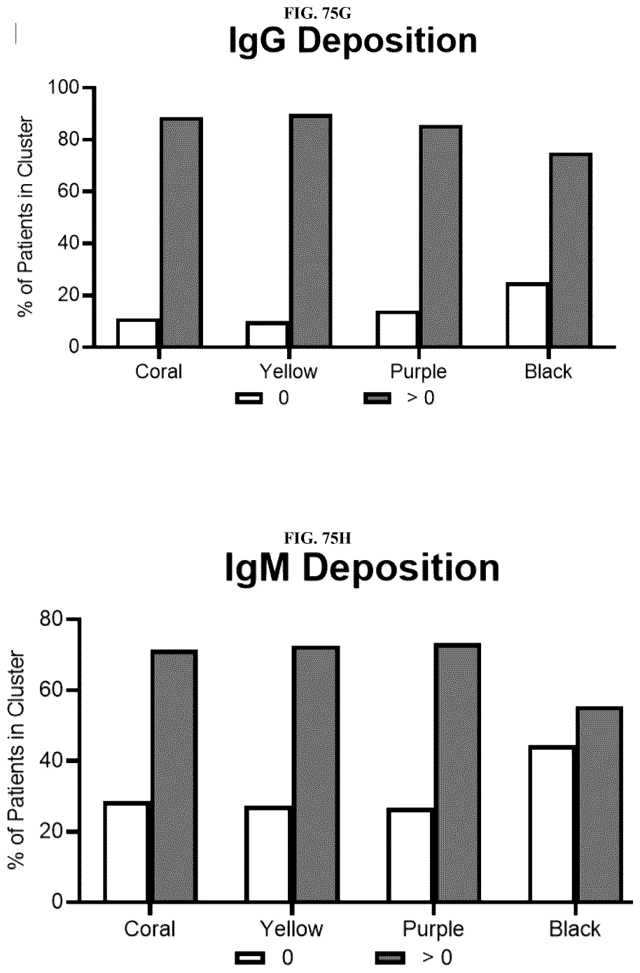

Results: FIGS. 75 A-B and 76A-I show clustering of metabolic and cellular GSVA enrichment scores in LN kidneys reveals four distinct endotypes of patients with LN. Analysis of LN gene expression with GSVA of kidney biopsy samples of 76 patients with LN (BH11201) revealed four distinct endotypes of LN (FIG. 74A), which could be ordered from least to most severe based on the profile of abnormal features (FIG. 74B). The least severe cluster (“coral”) exhibited minimal immune cell infiltrates or changes to kidney cell or metabolic signature expression. The second cluster (“yellow”) had increased expression of immune/inflammatory cell signatures, with minimal changes to kidney cell or metabolic signatures. The third cluster (“purple”) exhibited both increased expression of immune cell signatures and decreased expression of kidney cell and metabolic signatures. The final cluster (“black”) exhibited minimal immune cell gene expression, but decreased metabolism signatures and increased endothelial cell, fibroblast, and mesangial cell signatures. The columns represent individual patients that are grouped into four clusters (Coral, Yellow, Purple, and Black). The rows represent gene modules indicative of immune/inflammatory cells, non-hematopoietic cells, and cellular metabolism.

FIGS. 76A-I show comparison of the molecular endotypes, as shown in FIGS. 74A and B, for GSVA enrichment of signatures for specific (A-C) metabolism (D-F) non-hematopoietic cells, and (G-I) immune cells. Significant differences in mean GSVA enrichment score between Coral, the “least abnormal” LN endotype, and each other cluster were assessed by Brown-Forsythe and Welch ANOVA with Dunnett's T3 multiple comparisons. *, p<0.05, ***, p<0.001, ****, p<0.0001.

FIGS. 75A-H show Systemic disease activity correlates with molecular endotypes of LN, whereas histologic metrics do not. FIG. 75A shows ISN/RPS classes in 46 patients with LN. FIG. 75B shows positive or negative IgA deposition in 44 patients with LN. FIG. 75C shows distribution of active or inactive disease by SLEDAI in 32 patients with LN. FIG. 75D shows renal activity index in 49 patients with LN. FIG. 75E shows renal chronicity index in 49 patients with FIG. 75F shows proteinuria values (g/24 h) in 24 patients with LN. FIG. 75G shows positive or negative IgG deposition in 41 patients with LN. FIG. 75H positive or negative IgM deposition in 42 patients with LN. Of the LN samples with paired histology, 24/33 samples with proliferative LN were found in clusters with increased immune/inflammatory cell signatures (15/33 in the purple cluster, 9/33 in the yellow cluster) (FIG. 75A). Notably, the percentage of patients with IgA deposition on the glomerular basement membrane was highest in the purple cluster, and significantly lower in the coral cluster (FIG. 75B). Moreover, the percentage of patients with active disease determined by SLEDAI was lowest in the coral cluster (FIG. 75C). Mean renal activity and chronicity indices were not significantly different between clusters (FIG. 75D-E). In FIGS. 75 A, B, C, G and H significant differences in expected and observed frequencies between Coral, the “least abnormal” LN endotype, and all other clusters were evaluated by Chi Square Test. The likelihood (odds ratio) of having active disease in the Coral cluster was 0.06 (p=0.006) as compared to the three other clusters. The likelihood (odds ratio) of having proliferative nephritis, or positive IgA deposition, IgG deposition, or IgM deposition in the Coral cluster as compared to the other three clusters was not significant. In (FIGS. 75 A, B, C, G and H) significant associations between the categorical variables and all clusters (denoted with asterisks on the y-axis) were evaluated using Chi Square Test of Independence. In (FIGS. 75 D, E, and F) significant differences in enrichment of the clinical variable between each cluster and Coral was assessed by Brown-Forsythe and Welch ANOVA with Dunnett's T3 multiple comparisons. All data is for patients in dataset BH11201

FIG. 77 show clustering of GSVA enrichment scores into the four kidney-derived molecular clusters, using informative cellular and pathway signatures (Tables 25-1 to 25-32) in paired blood from patients with LN (BH11201). The columns represent individual patients that are grouped into four clusters (Coral, Yellow, Purple, Black). The rows represent gene modules indicative of immune/inflammatory cells and cellular pathways/processes.

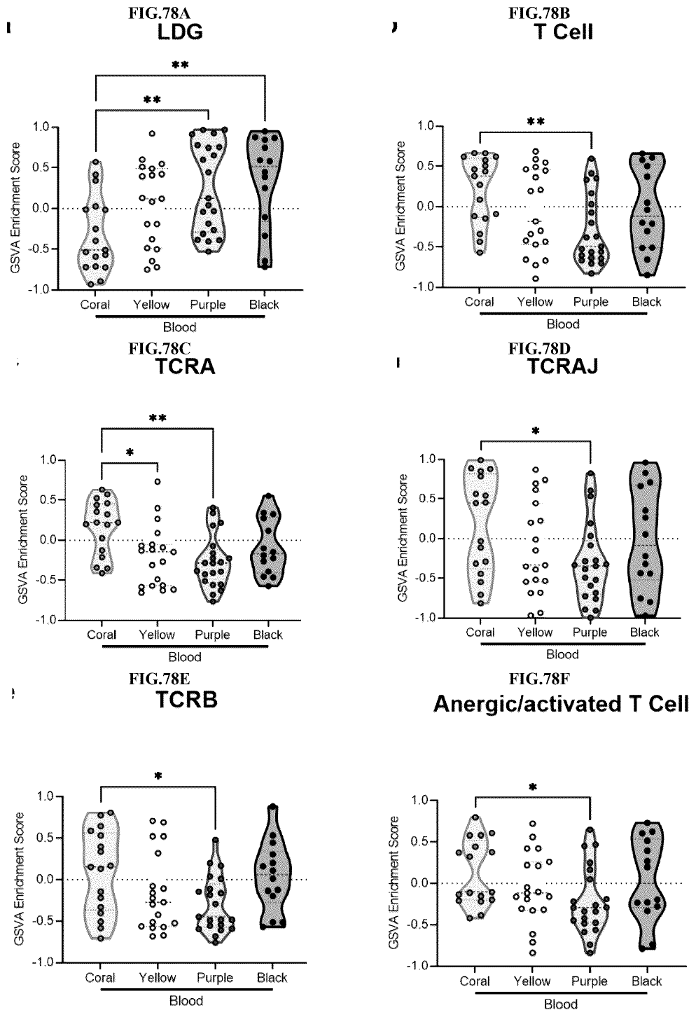

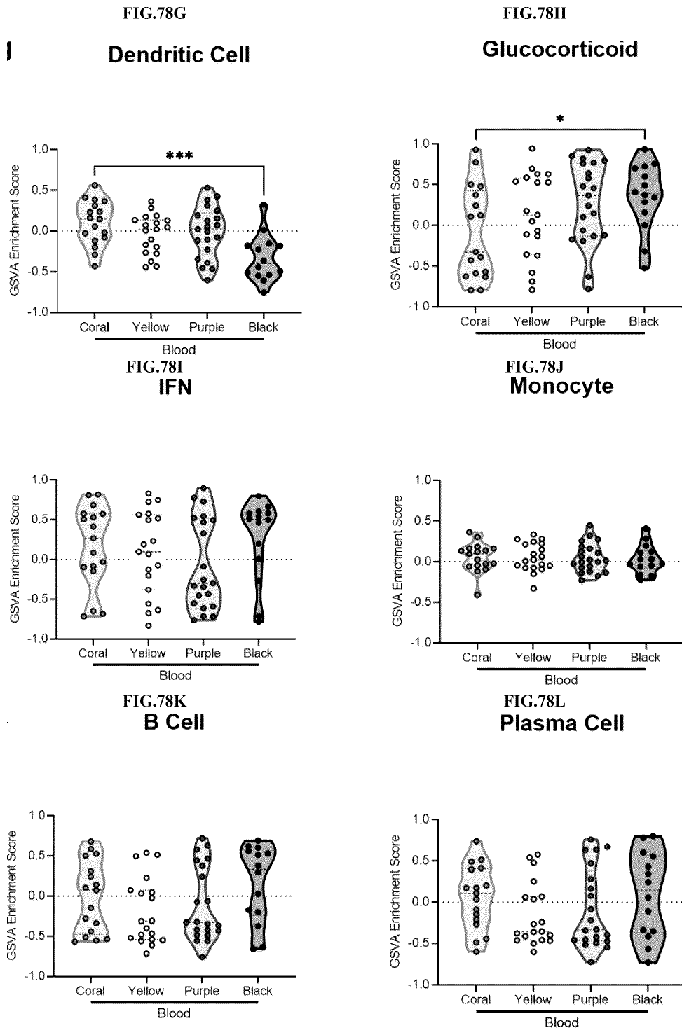

FIGS. 78A-L show analysis of paired blood of patients with LN demonstrates cluster-specific enrichment of LDG, T cell, dendritic cell, and glucocorticoid signatures. GSVA enrichment of (FIG. 78A) LDG, (FIG. 78B) T cell, (FIG. 78C) TCRA, (FIG. 78D) TCRAJ, (FIG. 78E) TCRB, (FIG. 78F) anergic/activated T cell, (FIG. 78G) dendritic cell, (FIG. 78H) glucocorticoid, (FIG. 781) interferon (IFN), (FIG. 78J) monocyte, (FIG. 78K) B cell, and (FIG. 78L) plasma cell signatures in the blood of 71 patients with LN (BH11201) are shown. X-axis clusters denote the cluster to which the sample belongs based upon analysis of paired kidney gene expression. Significant differences in enrichment of gene signatures between each cluster and Coral was assessed by Brown-Forsythe and Welch ANOVA with Dunnett's T3 multiple comparisons. *, p<0.05, **, p<0.01, ***, p<0.001. The glucocorticoid signature is derived from Northcott et al. (2).

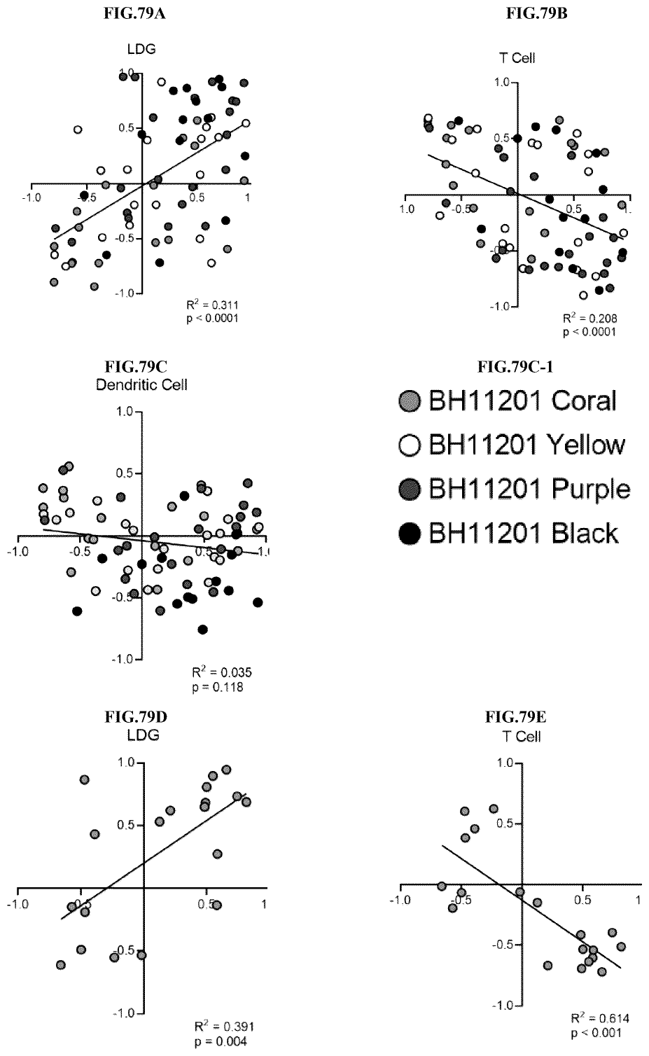

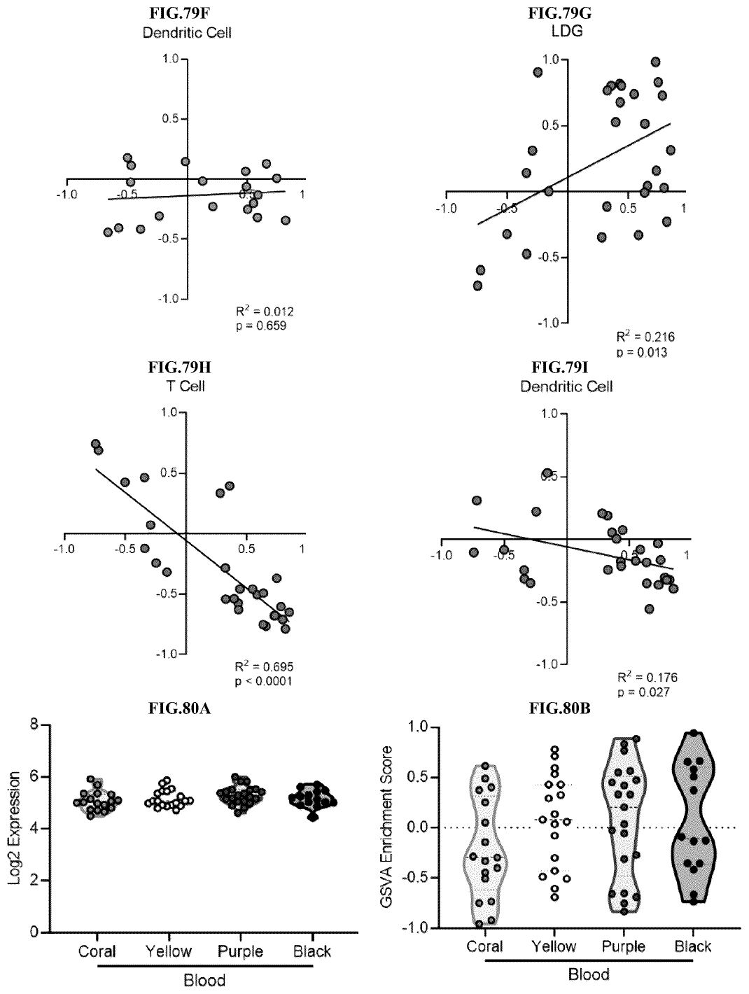

FIGS. 79A-I show the LDG and T cell signatures are consistently correlated with the glucocorticoid signature in the blood of patients with LN, whereas the dendritic cell signature is not. Linear regression of the glucocorticoid signature with the LDG, T cell, and dendritic cell signatures in the blood of patients with lupus nephritis for (FIGS. 79A-C) BH11201 (n=71), (FIGS. 79D-F) GSE49454 (n=19), and (FIGS. 79G-J) GSE99967 (n=28) is shown. The glucocorticoid signature is derived from Northcott et al. (2). In each of FIGS. 79-I the glucocorticoid signature is shown in the x-axis.

FIGS. 80A-B show the expression of erythropoietin (EPO) or a recombinant human erythropoietin (rHuEPO) signature in the blood of patients with LN is not associated with the molecular endotypes LN. (a) Log2 expression of EPO in the paired blood of 71 patients with LN. (b) GSVA enrichment of the rHuEPO signature in the paired blood of 71 patients with LN. The rHuEPO signature was derived from Wang et al (3), where differentially expressed genes were measured after administration of rHuEPO, and nine of the genes that were consistently expressed after rHuEPO administration comprised the signature.

Conclusion: Transcriptomic analyses suggest the existence of LN endotypes that progress from acute inflammatory to chronic kidney disease with little inflammation and marked kidney damage. These endotypes include: 1) minimal disease (coral cluster); 2) inflammatory disease without kidney cell damage or metabolic dysfunction (yellow cluster); 3) inflammatory disease with kidney cell and metabolic dysfunction (purple cluster); and 4) markedly decreased kidney cell and metabolic function with little inflammation (black cluster). There is an association between disease activity and molecular endotypes of LN, whereas there is minimal to no association between molecular endotypes and histologic phenotype. Analysis of the blood of patients with LN suggest that the two most severe molecular endotypes of LN have different profiles than patients with minimal disease. Although the LDG signature can relate to glucocorticoid treatment (4) and T cell lymphopenia is a marker of severe lupus in general (5), and both were correlated with the glucocorticoid signature, the combination suggests greater suspicion of progression to more severe LN. The dendritic cell signature was less likely to be associated with the glucocorticoid signature, suggesting it may be a promising blood biomarker of more severe LN. Although production of erythropoietin is known to decrease with chronic kidney disease (6), expression of EPO was unchanged in the blood across subsets. The molecular endotypes of LN may be an important way to stage LN and provide useful information to guide therapy.

Tables 23-1 to 23-28 show the GSVA gene sets.
The genes in each of Tables 23-1 to 23-28 can be used as effective biomarkers for classifying the LN disease state of the patient.

TABLE 23-1
Amino Acid Metabolism

AASS, AGXT, AGXT2, ALDH4A1, ALDH7A1, ARG1, GAD1, GLUD1,
GOT1, GPT2, GRHPR, IVD, MCCC2, OAT, OTC, OXCT1, PC,
PSAT1, SDS

TABLE 23-2
Fatty Acid Alpha Oxidation

HAAO, HACL1, PEX13, PHYH, SLC27A2

TABLE 23-3
Fatty Acid Beta Oxidation

ABCD1, ABCD2, ABCD3, ACAA2, ACACB, ACAD11, ACADL, ACADM, ACADS,
ACADVL, ACAT1, ACAT2, ACOX1, ACOX2, ACOX3, ACOXL, ACSBG2, ACSL5, ADIPOQ,
AKT2, AUH, BDH2, CPT1A, CPT2, CROT, DECR1, ECHDC1, ECHDC2, ECHS1, ECI1, ECI2,
EHHADH, ETFA, ETFB, ETFDH, FABP1, GCDH, HADH, HADHA, HADHB, HIBCH,
HSD17B4, IRS1, IRS2, IVD, LEP, PEX2, PEX5, PEX7, SESN2, SLC25A17, SLC27A2,
TWIST1

TABLE 23-4
Glycolysis

G6PC2, HKDC1, LDHAL6A, LDHAL6B, PFKFB2, PFKFB3, PFKFB4,
PKM, SLC2A1, SLC2A3, SLC2A4, SLC2A5

TABLE 23-5
Oxidative Phosphorylation

ATP5A1, ATP5B, ATP5D, ATP5E, ATP5F1, ATP5G1, ATP5G2, ATP5G3, ATP5H, ATP5I,
ATP5J, ATP5J2, ATP5L, ATP5O, ATP5S, BCS1L, CEP89, COA1, COA3, COA4, COA5,
COA6, COA7, COX10, COX10-AS1, COX11, COX14, COX15, COX16, COX17, COX18,
COX19, COX20, COX4I1, COX4I2, COX5A, COX5B, COX6A1, COX6A2, COX6B1,
COX6B2, COX6C, COX7A1, COX7A2, COX7A2L, COX7B, COX7B2, COX7C, COX8A,
COX8C, CYC1, CYCS, DNAJC15, MT-ATP6, MT-ATP8, MT-CO1, MT-CO2, MT-CO3, MT-
CYB, MT-ND1, MT-ND2, MT-ND3, MT-ND4, MT-ND4L, MT-ND5, MT-ND6, NDUFA1,
NDUFA10, NDUFA11, NDUFA12, NDUFA13, NDUFA2, NDUFA3, NDUFA4, NDUFA4L2,
NDUFA5, NDUFA6, NDUFA7, NDUFA8, NDUFA9, NDUFAB1, NDUFAF1, NDUFAF2,
NDUFAF3, NDUFAF4, NDUFAF5, NDUFAF6, NDUFAF7, NDUFAF8, NDUFB1, NDUFB10,
NDUFB11, NDUFB2, NDUFB2-AS1, NDUFB3, NDUFB4, NDUFB5, NDUFB6, NDUFB7,
NDUFB8, NDUFB9, NDUFC1, NDUFC2, NDUFS1, NDUFS2, NDUFS3, NDUFS4, NDUFS5,
NDUFS6, NDUFS7, NDUFS8, NDUFV1, NDUFV2, NDUFV3, NUBPL, OXA1L, RFESD,
SCO1, SCO2, SLC25A4, SURF1, TACO1, TIMMDC1, TMEM126B, TRAP1, TTC19, UQCC1,
UQCC2, UQCC3, UQCR10, UQCR11, UQCRB, UQCRC1, UQCRC2, UQCRFS1, UQCRH,
UQCRHL, UQCRQ

TABLE 23-6
Pentose Phosphate

G6PD, H6PD, PGD, PRPS1, PRPS1L1, PRPS2, RBKS, RGN, RPE,
RPIA, TALDO1, TKT, TKTL1, TKTL2

TABLE 23-7
TCA Cycle

ACO2, CS, DLAT, DLD, DLST, FH, GLUD1, IDH1, IDH2, IDH3A, IDH3B, IDH3G, MDH2,
MPC1, MPC2, OGDH, OGDHL, PDHA1, PDHA2, PDHB, PDHX, PDK1, PDK2, PDK3, PDK4,
PDP1, PDP2, PDPR, SDHA, SDHAF1, SDHAF2, SDHAF3, SDHAF4, SDHB, SDHC, SDHD,
SUCLA2, SUCLG1, SUCLG2, SUGCT

TABLE 23-8
Anergic or Activated T

CD160, CD244, CTLA4, ICOS, KLRG1, LAG3, PDCD1

TABLE 23-9
B Cell

AICDA, BANK1, BLK, BLNK, CD19, CD22, CD79A, CD79B, CLEC17A, CLNK, CR2,
DAPP1, DTX1, FAM129C, FAM30A, FCRL1, FCRL2, FCRL4, FCRL5, FCRLA, GAPT,
GNG7, GPR183, HLA-DOA, HLA-DOB, IGHD, IGHM, IRF4, LINC00926, LY6D, MEF2B,
MS4A1, MSC, PAX5, PLCL2, POU2AF1, PRKCH, SH2B2, SIGLEC6, ST6GAL1,
TNFRSF13B, TNFRSF13C, VPREB1, VPREB3

TABLE 23-10
Dendritic Cell

CD1B, CD1C, CD1E, CD207, CLEC10A, CLEC9A, GPR31, MRC1,
XCR1

TABLE 23-11
GC B Cell

FCRLB, GCSAM, KLHL6, LRMP, NUGGC, P2RY8, PARPBP, RGS13

TABLE 23-12
Granulocyte

ADGRG5, ARG1, BPI, CAMP, CD177, CLC, CRISP3, CTSS, CXCR2,
DEFA3, FPR2, LTB4R, MMP25, MMP8, MS4A2, OLFM4, OSM,
PGLYRP1, PRG2, PRTN3, S100A12

TABLE 23-13
LDG

AZU1, CEACAM3, CEACAM4, CEACAM5, CEACAM6, CEACAM8,
CTSG, DEFA4, ELANE, MPO, RETN, RNASE3

TABLE 23-14
Monocyte/Myeloid Cell

ACE, ADAM8, ADAMDEC1, ADGRE1, ADGRE2, ADGRE3, AIF1, AOAH, APOBEC3B,
APOBEC3G, APOBR, APOC1, ART4, BMX, C1QA, C1QB, C1QC, C2, C4A, C4B, C4BPA,
C4BPB, C5, C6, C8A, C9, CCL17, CCL18, CCL22, CCL28, CCL7, CCL8, CD101, CD14,
CD163, CD209, CD300C, CD300E, CD300LF, CD33, CD5L, CD68, CD80, CFB, CFD, CFP,
CHI3L1, CHIT1, CLEC12A, CLEC12B, CLEC16A, CLEC1A, CLEC2B, CLEC4A, CLEC4D,
CLEC4E, CLEC4G, CLEC5A, CLEC6A, CLEC7A, CSF1R, CSF2RA, CSF2RB, CSF3R, CST3,
CXCL1, CXCL10, CXCL11, CXCL13, CXCL8, CXCL9, CYBA, CYBB, F12, FCER1G,
FCGR1A, FCGR1B, FCGR2A, FCGR2B, FCGR3A, FCGR3B, FFAR2, FGR, FLT3, HMMR,
HVCN1, IFI30, IFNL1, IFNL2, IFNL3, IGSF6, IL10RA, IL12A, IL12B, IL1A, IL1B, IL1F10,
IL1RAP, IL1RN, IL20, IL23A, IL26, IL27, IL31RA, IL37, ITGAX, LAMP3, LGALS12,
LGALS4, LGALS9, LGALS9B, LGALS9C, LILRA1, LILRA2, LILRA3, LILRA5, LILRA6,
LILRB1, LILRB2, LILRB3, LILRB4, LILRB5, LMNB1, LY86, LYVE1, LYZ, MARCO, MEFV,
MEGF10, MERTK, MFGE8, MNDA, MPEG1, MS4A4A, MS4A6A, MS4A7, MSR1, NLRP12,
NLRP3, NOD2, NTSR1, OCM, OLR1, OSCAR, OTOF, PDCD1LG2, PECAM1, PILRA,
PLA2G2D, PLEK, PRAM1, PYHIN1, RNASE6, RNASE7, S100A8, S100A9, S1PR5, SCARB1,
SCARF2, SECTM1, SEMA4A, SERPINB9, SERPING1, SIGLEC1, SIGLEC10, SIGLEC14,
SIGLEC5, SIGLEC7, SKAP2, SLAMF8, SLC11A1, SLITRK4, SLPI, SMPDL3B, SPI1, SPIC,
STAB2, STAP2, TEK, TGM2, TIMD4, TLR2, TLR8, TNF, TNFAIP8L2, TNFRSF1B,
TNFSF13B, TNFSF14, TNIP3, TREM1, TREML2, TREML4, TULP1, TYROBP, UBD,
UNC93B1, VENTX, VSIG4, VSTM1

TABLE 23-15
NK Cell

KIR2DL4, KLRC3, KLRF1, NCAM1, NCR1, SH2D1B, TNFSF11, TXK

TABLE 23-16
pDC

CLEC4C, IRF7, LILRA4, NRP1, PACSIN1, PLAC8, PTCRA,
SERPINF1, SLC15A4, TCF4

TABLE 23-17
Plasma Cell

DERL3, FANCI, GPRC5D, IGH, IGHA1, IGHA2, IGHD2-15, IGHD2-21, IGHD3-10, IGHD3-
16, IGHD3-3, IGHE, IGHG1, IGHG2, IGHG3, IGHG4, IGHGP, IGHJ1, IGHJ2, IGHJ3, IGHJ4,
IGHJ5, IGHJ6, IGHMBP2, IGHV1-18, IGHV1-2, IGHV1-24, IGHV1-3, IGHV1-45, IGHV1-46,
IGHV1-58, IGHV1-69-2, IGHV2-26, IGHV2-5, IGHV2-70, IGHV3-11, IGHV3-13, IGHV3-15,
IGHV3-16, IGHV3-20, IGHV3-21, IGHV3-23, IGHV3-33, IGHV3-35, IGHV3-38, IGHV3-43,
IGHV3-47, IGHV3-48, IGHV3-49, IGHV3-53, IGHV3-54, IGHV3-7, IGHV3-72, IGHV3-73,
IGHV3-74, IGHV3OR16-8, IGHV4-28, IGHV4-30-2, IGHV4-31, IGHV4-34, IGHV4-39,
IGHV4-59, IGHV4-61, IGHV5-51, IGHV5-78, IGHV6-1, IGHV7-34-1, IGHV7-81, IGK, IGKC,
IGKJ1, IGKJ2, IGKJ3, IGKJ4, IGKJ5, IGKV@, IGKV1-16, IGKV1-17, IGKV1-27, IGKV1-5,
IGKV1-6, IGKV1-9, IGKV1D-16, IGKV1D-17, IGKV1D-27, IGKV1D-43, IGKV1D-8,
IGKV2D-24, IGKV2D-26, IGKV3-20, IGKV3-7, IGKV3D-20, IGKV3D-7, IGKV4-1, IGKV5-2,
IGL, IGLC1, IGLC2, IGLC6, IGLC7, IGLJ3, IGLJ6, IGLL1, IGLL3P, IGLL5, IGLV@,
IGLV10-54, IGLV1-36, IGLV1-40, IGLV1-44, IGLV1-47, IGLV1-50, IGLV2-11, IGLV2-14,
IGLV2-18, IGLV2-23, IGLV2-5, IGLV2-8, IGLV3-1, IGLV3-10, IGLV3-12, IGLV3-16, IGLV3-
19, IGLV3-21, IGLV3-22, IGLV3-25, IGLV3-27, IGLV3-9, IGLV4-3, IGLV4-60, IGLV4-69,
IGLV5-37, IGLV5-45, IGLV5-48, IGLV6-57, IGLV7-43, IGLV7-46, IGLV8-61, IGLV9-49,
IGLVI-56, IGLVI-70, MZB1, PCLAF, TNFRSF17

TABLE 23-18
Platelet

GP1BA, GP9, ITGA2B, PF4, PF4V1, PPBP

TABLE 23-19
T Cell

CD247, CD3D, CD3E, CD3G, CD8A, CD8B, TRAC, TRAT1,
TRAV20, TRBC1, TRDC

TABLE 23-20
Endothelial Cell

DLC1, ECSCR, EMCN, FLT1, KDR, LDB2, LRRC32, MEIS2,
PLAT, PTPRB, SELE, TM4SF1, TM4SF18, VWF

TABLE 23-21
Fibroblast

43894, ADAM33, ADAMTS6, AGTR1, ALPK2, ANGPTL2, ANKRD45, ANO2, ANPEP,
ARMC9, ASPN, BDKRB2, BDNF, BMPER, C11orf87, C1R, C1S, CATSPER3, CCDC102B,
CCDC80, CCDC81, CEMIP, CHAC1, CHRM2, CLMP, CNN1, COL14A1, COL3A1, COL5A1,
COLEC10, CPXM2, CPZ, CRABP2, CXCL12, DDR2, DKK1, DMRTA1, EGFL6, ELOVL2,
EMILIN1, FAM180A, FBLN7, FBN1, FGF5, FMN2, FOXF2, FST, FSTL1, GFRA1, GLIS1,
GLT8D2, GPR176, GREM1, GREM2, GRIK2, GUCY1A2, HSD17B2, HSPA2, HSPB3, IL19,
IQCD, KCNMB2, KIRREL3, KRT34, KRTAP1-5, KRTAP3-1, L1CAM, LAYN, LMOD1,
LOXL4, LUM, LY6K, MFAP4, MFAP5, MGARP, MGP, MKX, MMP2, MXRA5, MXRA8,
MYPN, NEXN, NFASC, NID2, NTF3, OLFML3, P3H3, P4HA3, PAMR1, PAX3, PCDHGA2,
PCDHGA3, PCDHGA7, PDE8B, PDGFRA, PDGFRL, PDZRN3, PLA2R1, PLEKHA4, PLPP4,
PLPPR4, PRKG1, PSG5, PTPRQ, PTRF, SEMA3A, SEMA5A, SEMA6D, SHOX, SLC16A2,
SMIM2, SPARCL1, SPHKAP, SSTR1, STC1, STXBP6, SUSD5, SVEP1, TBX15, THBS2,
TIMP2, TIMP3, TMEM119, TMEM130, TMEM47, TRHDE, TRPC4, UACA, UBL4B, VAT1L,
VEGFC, WNT5A, WNT5B, ZFPM2

TABLE 23-22
Kidney Cell

A2LD1, ACE2, ACOT4, ACP6, ACSF2, ADCY5, ADGRF1, ADHFE1, AGBL3, AGMAT,
AGR2, AJAP1, AKR7A3, ALDH8A1, ARL4C, ARSE, ARSF, BICC1, BICDL1, C16orf11,
C21orf62, C6orf47, CALCRL, CCDC106, CCDC160, CD24, CDHR5, CDKL2, CHDH, CHST9,
CLDN10, CLDN16, CLDN2, CLRN3, COL4A3, COL4A4, COL4A5, COMTD1, COQ8A,
CPVL, CTXN3, CXorf57, CYP27B1, CYP4A22, CYS1, DAO, DCDC2, DCXR, DHDH,
DMGDH, EFHD1, EME2, EN2, ERVMER34-1, ESM1, ETNK2, FAM110C, FAM134B,
FAM151A, FAM184A, FANK1, FNDC8, FOXI1, FRMD7, FTCD, G6PC, GGH, GLOD5, GNE,
HABP2, HAO2, HAVCR1, HEY1, HIST1H4D, HMX1, HNF1A, HNF1B, HOGA1, HOOK1,
HOXD3, HPD, HS3ST1, HS6ST2, HSPA12A, HYKK, IL17RB, INSRR, IRX5, ITGB8, KCNH6,
KCNJ11, KCTD14, KIF12, LAMB2, LGSN, LHX1, LIPG, LRP2, LRRC19, MDC1, MZF1,
NME5, NOS1, NR1H4, OR2T10, OSBPL10, OVGP1, P3H2, PADI1, PANK1, PAX8, PCK1,
PCSK5, PDZD3, PDZK1IP1, PKHD1, PKLR, PLEKHA1, PLLP, PLPPR1, POU3F3, PPT2,
PRKAA2, PRODH, PRODH2, PROM1, PROZ, PRRG1, PTCHD1, RALYL, RANBP3L, RARB,
RARRES3, RASIP1, RBM20, REN, RHBG, RIDA, RNF182, RNF212B, RPRM, RSPH1, RYR3,
SALL1, SCEL, SCNN1G, SFXN2, SGK2, SGPP2, SHISA3, SIM2, SLC10A2, SLC13A3,
SLC16A4, SLC16A5, SLC17A1, SLC17A3, SLC22A11, SLC22A24, SLC22A6, SLC22A7,
SLC28A1, SLC30A2, SLC34A3, SLC3A1, SLC47A2, SLC4A4, SLC6A19, SLC7A13, SLC7A7,
SLC7A9, SLCO4A1, SMTNL2, SOD3, SOSTDC1, SPON1, SYT9, SYTL2, TAGLN3, TLN2,
TMEM207, TMPRSS4, TPD52L1, TRABD2B, TRPV5, TSPAN1, TSPAN12, UGT1A9,
UGT2A3, UGT3A1, UGT8, UPK1B, UPP2, VEPH1, WIF1, WNK4, XPNPEP2, ZBED2,
ZNF324B, ZNF331, ZNF837, ZPLD1, ZSCAN18

TABLE 23-23
Kidney Distal Tubule

ATP6V0A4, ATP6V0D2, C14orf105, CALB1, CDH1, CDH16,
CLCNKA, CLDN8, EMX1, EPCAM, KCNJ1, PTH1R, PVALB,
SCIN, SCNN1A, SIM1, SLC12A1, SLC6A13, SULT1C2,
TFCP2L1, TMEM213, TMEM72, UMOD, WFDC2

TABLE 23-24
Kidney Loop of Henle Cell

CD9, CLCNKB, KCNJ16, KNG1, S100A6, SPP1

TABLE 23-25
Kidney Proximal Convoluted Tubule

ACSM2A, ACSM2B, ACY1, ANK2, AQP1, ASS1, BBOX1, BHMT,
BHMT2, C10orf126, CMBL, CRYAA, CRYAB, CUBN, CXCL14,
DDAH1, FMO1, GALNT14, GLYAT, GPX3, MIOX, NAT8, NOX4,
PAH, PAX2, PDZK1, RAB11FIP3, RNF152, SLC12A3, SLC13A1,
SLC16A12, SLC22A12, SLC22A13, SLC22A2, SLC22A8, SLC34A1,
SLC36A2, SLC5A2, SLC6A18, TMEM174, TMEM52B, UGT2B7

TABLE 23-26
Kidney Tubule Collecting Duct Cell

AQP2, CLDN4, COL4A1, CPE, CYR61, FXYD4, GLIS3,
MACC1, MITF, PIGR, SLC4A9, WWC1

TABLE 23-27
Mesangial Cell

ACTA2, ACTN1, ADAMTSL4, AKAP12, AMOTL2, ANGPT2,
ARID5B, CALD1, CSTB, CTGF, DYRK1A, ECM1, FGF1,
FILIP1L, FLNA, FN1, GATA3, HOPX, ITGA8, LATS2,
LRP5, MAP3K20, MEF2C, MIB1, NCOA2, NOCT, NR4A2,
OGT, PAWR, PDE3A, PDGFB, PDGFRB, PER3, PLAU,
PML, PTN, RASD1, ROCK1, SERPINE2, SFRP2,
SH3BGRL, SMARCA5, SNCG, TBX18, THY1, TNS1,
TPM2, TRIP4, YAP1

TABLE 23-28
Podocyte

NPHS1, NPHS2, PODXL, SYNPO, WT1

TABLE 24
Kidney and blood datasets for patients with
LN, SLE, or controls used for the analysis.

| | | | Con- | |
|---|---|---|---|---|
| Dataset | Organization | Tissue | trol | Lupus |
| BH11201 | Shanghai JiaoTong | Kidney | 8 | 76 (LN) |
| | University | | | |
| | School of Medicine | | | |
| BH11201 | Shanghai JiaoTong | Blood | 16 | 71 (LN) |
| | University | | | |
| | School of Medicine | | | |
| GSE49454 | Baylor Institute for | Blood | 10 | 30 (Non-renal SLE) |
| | Immunology Research | | | 19 (LN) |
| GSE99967 | Ontario Institute for | Blood | 16 | 12 (Non-renal SLE) |
| | Cancer | | | 28 (LN) |
| | Research | | | |

Tables 25-1 to 25-32: 32 Genes Modules listing 989 Genes. Genes are Listed by: Gene Symbol|Gene Entrez ID 11)
The genes in each of Tables 25-1 to 25-32 can be used as effective biomarkers for classifying the LN disease state of the patient.

TABLE 25-1
Module: Anergic/Activated T cells

CD160 | 11126 || CD244 | 51744 || CTLA4 | 1493 || HAVCR2 | 84868 || ICOS | 29851 ||
KLRG1 | 10219 || LAG3 | 3902 || PDCD1 | 5133 ||

TABLE 25-2
Module: Anti inflammation

IL1RN | 3557 || SOCS3 | 9021 || TNFAIP3 | 7128 ||

TABLE 25-3
Module: B cells

BANK1 | 55024 || BLK | 640 || BLNK | 29760 || CD19 | 930 || CD22 | 933 || CD79A | 973 ||
CD79B | 974 || DAPP1 | 27071 || FCRL1 | 115350 || FCRL2 | 79368 || FCRL3 | 115352 ||
FCRLA | 84824 || GON4L | 54856 || GPR183 | 1880 || IGHD | 3495 || IGHM | 3507 ||
KLHL6 | 89857 || MS4A1 | 931 || PAX5 | 5079 || PLCL2 | 23228 || SH3BP5 | 9467 ||
VPREB1 | 7441 || ZNF318 | 24149 ||

TABLE 25-4
Module: Cell Cycle

ASPM | 259266 || AURKA | 6790 || AURKB | 9212 || BRCA1 | 672 || CCNB1 | 891 ||
CCNB2 | 9133 || CCNE1 | 898 || CDC20 | 991 || CENPM | 79019 || CEP55 | 55165 || E2F3 |
1871 || GINS2 | 51659 || MCM10 | 55388 || MCM2 | 4171 || MKI67 | 4288 || NCAPG |
64151 || NDC80 | 10403 || PTTG1 | 9232 || TYMS | 7298 ||

TABLE 25-5
Module: Dendritic

CLEC10A | 10462 || CLEC12A | 160364 || CLEC9A | 283420 || CSF1R |
1436 || IGIP | 492311 || LILRA4 | 23547 || LY75 | 4065 || XCR1 | 2829 ||

TABLE 25-6
Module: gd T cells

TRG-AS1 | 100506776 || BLK | 640 || TARP | 445347 || TRDC | 28526 ||
CD3E | 916 || CD3G | 917 ||

TABLE 25-7
Module: Granulocyte
Feature co-efficient = 0.48; Absolute feature co-efficient = 0.48

CD177 | 57126 || CLC | 1178 || CTSS | 1520 || CXCR2 | 3579 || DEFA1 |
1667 || FUT7 | 2529 || LTB4R | 1241 || MMP25 | 64386 || OSM |
5008 || RETN | 56729 ||

TABLE 25-8
Module: IFN

EIF2AK2 | 5610 || GBP1 | 2633 || GBP2 | 2634 || GBP4 | 115361 || HERC5 | 51191 ||
HERC6 | 55008 || IFI27 | 3429 || IFI30 | 10437 || IFI35 | 3430 || IFI44 | 10561 || IFI44L |
10964 || IFI6 | 2537 || IFIT1 | 3434 || IFIT2 | 3433 || IFIT3 | 3437 || IFIT5 | 24138 || IFITM1
| 8519 || IFITM2 | 10581 || IFITM3 | 10410 || ISG15 | 9636 || ISG20 | 3669 || MX1 | 4599 ||
MX2 | 4600 || OAS1 | 4938 || OAS2 | 4939 || OAS3 | 4940 || OASL | 8638 || RSAD2 |
91543 || SAMD9 | 54809 || SAMD9L | 219285 || SP100 | 6672 || SP110 | 3431 ||

TABLE 25-9
Module: IG CHAINS

IGHA1 | 3493 || IGHA2 | 3494 || IGHD2-15 | 28503 || IGHD2-2 | 28505 || IGHD2-21 |
28502 || IGHD3-10 | 28499 || IGHD3-16 | 28498 || IGHD3-3 | 28501 || IGHD3-9 | 28500 ||
IGHG1 | 3500 || IGHG2 | 3501 || IGHG3 | 3502 || IGHG4 | 3503 || IGHJ1 | 28483 || IGHJ2 |
28481 || IGHJ3 | 28479 || IGHJ4 | 28477 || IGHJ5 | 28476 || IGHJ6 | 28475 || IGHV1-18 |
28468 || IGHV1-2 | 28474 || IGHV1-24 | 28467 || IGHV1-3 | 28473 || IGHV1-45 | 28466 ||
IGHV1-46 | 28465 || IGHV1-58 | 28464 || IGHV1-69-2 | 28458 || IGHV2-26 | 28455 ||
IGHV2-5 | 28457 || IGHV2-70 | 28454 || IGHV3-13 | 28449 || IGHV3-15 | 28448 ||
IGHV3-20 | 28445 || IGHV3-21 | 28444 || IGHV3-23 | 28442 || IGHV3-33 | 28434 ||
IGHV3-43 | 28426 || IGHV3-48 | 28424 || IGHV3-49 | 28423 || IGHV3-53 | 28420 ||
IGHV3-62 | 28416 || IGHV3-64 | 28414 || IGHV3-7 | 28452 || IGHV3-72 | 28410 ||
IGHV3-73 | 28409 || IGHV3-74 | 28408 || IGHV4-28 | 28400 || IGHV4-30-2 | 28398 ||
IGHV4-34 | 28395 || IGHV4-39 | 28394 || IGHV4-59 | 28392 || IGHV4-61 | 28391 ||
IGHV5-51 | 28388 || IGHV6-1 | 28385 || IGHV7-81 | 28378 || IGKC | 3514 || IGKJ1 |
28950 || IGKJ2 | 28949 || IGKJ3 | 28948 || IGKJ4 | 28947 || IGKJ5 | 28946 || IGKV1-16 |
28938 || IGKV1-17 | 28937 || IGKV1-27 | 28935 || IGKV1-5 | 28299 || IGKV1-6 | 28943 ||
IGKV1-9 | 28941 || IGKV1D-16 | 28901 || IGKV1D-17 | 28900 || IGKV1D-43 | 28891 ||
IGKV1D-8 | 28904 || IGKV2-24 | 28923 || IGKV2D-26 | 28884 || IGKV2D-29 | 28882 ||
IGKV2D-30 | 28881 || IGKV3-20 | 28912 || IGKV3D-20 | 28874 || IGKV3D-7 | 28877 ||
IGKV4-1 | 28908 || IGKV5-2 | 28907 || IGLC2 | 3538 || IGLC7 | 28834 || IGLJ6 | 28828 ||
IGLV10-54 | 28772 || IGLV1-36 | 28826 || IGLV1-40 | 28825 || IGLV1-47 | 28822 ||
IGLV2-11 | 28816 || IGLV2-18 | 28814 || IGLV2-23 | 28813 || IGLV2-33 | 28811 || IGLV2-
8 | 28817 || IGLV3-1 | 28809 || IGLV3-10 | 28803 || IGLV3-12 | 28802 || IGLV3-16 | 28799
|| IGLV3-19 | 28797 || IGLV3-21 | 28796 || IGLV3-25 | 28793 || IGLV3-27 | 28791 ||
IGLV3-32 | 28787 || IGLV4-3 | 28786 || IGLV4-60 | 28785 || IGLV4-69 | 28784 || IGLV5-
37 | 28783 || IGLV5-45 | 28781 || IGLV6-57 | 28778 || IGLV7-43 | 28776 || IGLV8-61 |
28774 || IGLV9-49 | 28773 || IGLVI-70 | 28763 ||

TABLE 25-10
Module: IL1 Pathway

CHUK | 1147 || IKBKB | 3551 || NFKB1 | 4790 || MAP2K1 | 5604 || MAP2K4 | 6416 ||
IKBKG| 8517 || IL1A | 3552 || IL1R1 | 3554 || IRAK1 | 3654 || MAP3K3 | 4215 || MYD88
| 4615 || MAP2K6 | 5608 || MAP3K7 | 6885 || TRAF6 | 7189 || TAB1 | 10454 || TAB2 |
23118 || IRAK4 | 51135 || TOLLIP | 54472 || MAP3K8 | 1326 || IL1RAP | 3556 || IRAK2 |
3656 || MAPK8 | 5599 || RELA | 5970 || TAB3 | 257397 || IL1B | 3553 || UBE2N | 7334 ||
SQSTM1 | 8878 || IRAK3 | 11213 ||

TABLE 25-11
Module: IL23 Complex

IL12B | 3593 || IL12RB1 | 3594 || IL23A | 51561 || IL23R | 149233 ||

TABLE 25-12
Module: Immunoproteosome

PSMB10 | 5699 || PSMB8 | 5696 || PSMB9 | 5698 ||

TABLE 25-13
Module: Inflammasome

AIM2 | 9447 || CASP1 | 834 || CASP5 | 838 || CTSB | 1508 || GSDMB |
55876 || GSDMD | 79792 || NAIP | 4671 || NEK7 | 140609 || NLRC4 |
58484 || NLRP1 | 22861 || NLRP3 | 114548 || NOD2 | 64127 || P2RX7 |
5027 || PANX1 | 24145 || PYCARD | 29108 || RIPK1 | 8737

TABLE 25-14
Module: Inflammatory Cytokines

IL1A | 3552 || CXCL10 | 3627 || CXCL11 | 6373 || CXCL9 | 4283 ||
TNFSF13B | 10673 || CCL8 | 6355 || TNF | 7124 || IL1B | 3553 ||
IL18 | 3606 ||

TABLE 25-15
Module: Inhibitory Macs

AOAH | 313 || BACH1 | 571 || CD200R1 | 131450 || CD300A | 11314 ||
CD163 | 9332 || CLEC7A | 64581 || SCARB1 | 949 || MS4A4A | 51338 ||
IL10 | 3586 ||

TABLE 25-16
Module: LDG

AZU1 | 566 || CAMP | 820 || CEACAM6 | 4680 || CEACAM8 | 1088 ||
CTSG | 1511 || DEFA4 | 1669 || ELANE | 1991 || LCN2 | 3934 ||
LTF | 4057 || MPO | 4353 || OLFM4 | 10562 || RNASE3 | 6037 ||

TABLE 25-17
Module: MHC II

HLA-DMA | 3108 || HLA-DMB | 3109 || HLA-DPA1 | 3113 || HLA-DPB1 | 3115 || HLA-
DPB2 | 3116 || HLA-DQA1 | 3117 || HLA-DQA2 | 3118 || HLA-DQB1 | 3119 || HLA-
DQB2 | 3120 || HLA-DRA | 3122 || HLA-DRB1 | 3123 || HLA-DRB3 | 3125 || HLA-DRB4
| 3126 || HLA-DRB5 | 3127 || HLA-DRB6 | 3128 ||

TABLE 25-18
Module: Monocyte

ACE | 1636 || ADAM8 | 101 || ADAMDEC1 | 27299 || ADGRE1 | 2015 || ADGRE2
30817 || APOBEC3B | 9582 || APOBEC3G | 60489 || APOBR | 55911 || ART4 | 420 ||
C1QA | 712 || C1QC | 714 || C2 | 717 || C4A | 720 || C4B | 721 || C4BPA | 722 || C4BPB |
725 || C5 | 727 || C6 | 729 || C8A | 731 || C9 | 735 || CCL17 | 6361 || CCL18 | 6362 || CCL22
| 6367 || CCL28 | 56477 || CCL7 | 6354 || CCL8 | 6355 || CD14 | 929 || CD163 | 9332 ||
CD209 | 30835 || CD300C | 10871 || CD300E | 342510 || CD5L | 922 || CD80 | 941 || CFB |
629 || CFD | 1675 || CFP | 5199 || CHI3L1 | 1116 || CHIT1 | 1118 || CLEC12A | 160364 ||
CLEC12B | 387837 || CLEC4D | 338339 || CLEC4E | 26253 || CLEC5A | 23601 ||
CLEC7A | 64581 || CLEC9A | 283420 || CSF1R | 1436 || CXCL1 | 2919 || CXCL10 | 3627
|| CXCL11 | 6373 || CXCL13 | 10563 || CXCL8 | 3576 || CXCL9 | 4283 || CYBB | 1536 ||
F12 | 2161 || FCGR3B | 2215 || FFAR2 | 2867 || HMMR | 3161 || HVCN1 | 84329 || IGSF6 |
10261 || IL10RA | 3587 || IL12A | 3592 || IL12B | 3593 || IL1RAP | 3556 || IL20 | 50604 ||
IL23A | 51561 || IL27 | 246778 || IL31RA | 133396 || LAMP3 | 27074 || LGALS12 | 85329
|| LGALS4 | 3960 || LGALS9 | 3965 || LGALS9B | 284194 || LGALS9C | 654346 ||
LILRA2 | 11027 || LILRA5 | 353514 || LILRA6 | 79168 || LILRB5 | 10990 || LMNB1 |
4001 | LY86 | 9450 | LYZ | 4069 | MARCO | 8685 | MEGF10 | 84466 | MERTK | 10461
|| MFGE8 | 4240 || MPEG1 | 219972 || MRC1 | 4360 || MS4A4A | 51338 || MSR1 | 4481 ||
NTSR1 | 4923 || OCM | 654231 || OLR1 | 4973 || OSCAR | 126014 || OTOF | 9381 ||
PDCD1LG2 | 80380 || PILRA | 29992 || PLA2G2D | 26279 || PLA2G5 | 5322 || PYHIN1 |
149628 || S100A8 | 6279 || S100A9 | 6280 || S1PR5 | 53637 || SCARB1 | 949 || SCARF2 |
91179 || SECTM1 | 6398 || SEMA4A | 64218 || SERPINB9 | 5272 || SERPING1 | 710 ||
SIGLEC1 | 6614 || SIGLEC14 | 100049587 || SIGLEC5 | 8778 || SIGLEC7 | 27036 ||
SLC11A1 | 6556 || SLITRK4 | 139065 || SMPDL3B | 27293 || SPIC | 121599 || STAB2 |
55576 || STAP2 | 55620 || TEK | 7010 || TGM2 | 7052 || TIMD4 | 91937 || TLR2 | 7097 ||
TLR8 | 51311 || TNF | 7124 || TNFAIP8L2 | 79626 || TNFRSF1B | 7133 || TNIP3 | 79931 ||
TULP1 | 7287 || UBD | 10537 || VENTX | 27287 || VSTM1 | 284415 ||

TABLE 25-19
Module: Neutrophil

ABHD3 | 171586 || ADAM8 | 101 || CD177 |
57126 || CD83 | 9308 || CLEC6A | 93978 ||
LY6E | 4061 || S100A6 | 6277 ||

TABLE 25-20
Module: NK

KLRF1 | 51348 || NCAM1 | 4684 || NCR1 | 9437
|| NCR3 | 259197 | | SH2D1B | 117157 ||

TABLE 25-21
Module: Oxidative Phosphorylation

ATP5A1 | 498 || ATP5B | 506 || ATP5D | 513 || ATP5E | 514 || ATP5F1 | 515 || ATP5G1 |
516 || ATP5G2 | 517 || ATP5G3 | 518 || ATP5H | 10476 || ATP5I | 521 || ATP5J | 522 ||
ATP5J2 | 9551 || ATP5L | 10632 || ATP5O | 539 || ATP5S | 27109 || BCS1L | 617 || CEP89
| 84902 || COA1 | 55744 || COA3 | 28958 || COA4 | 51287 || COA5 | 493753 || COA6 |
388753 || COA7 | 65260 || COX10 | 1352 || COX10-AS1 | 100874058 || COX11 | 1353 ||
COX14 | 84987 || COX15 | 1355 || COX16 | 51241 || COX17 | 10063 || COX18 | 285521 ||
COX19 | 90639 || COX20 | 116228 || COX4I1 | 1327 || COX4I2 | 84701 || COX5A | 9377 ||
COX5B | 1329 || COX6A1 | 1337 || COX6A2 | 1339 || COX6B1 | 1340 || COX6B2 |
125965 || COX6C | 1345 || COX7A1 | 1346 || COX7A2 | 1347 || COX7A2L | 9167 ||
COX7B | 1349 || COX7B2 | 170712 || COX7C | 1350 || COX8A | 1351 || COX8C | 341947
|| CYC1 | 1537 || CYCS | 54205 || DNAJC15 | 29103 || MT-ATP6 | 4508 || MT-ATP8 |
4509 || MT-CO1 | 4512 || MT-CO2 | 4513 || MT-CO3 | 4514 || MT-CYB | 4519 || MT-ND1
| 4535 || MT-ND2 | 4536 || MT-ND3 | 4537 || MT-ND4 | 4538 || MT-ND4L | 4539 || MT-
ND5 | 4540 || MT-ND6 | 4541 || NDUFA1 | 4694 || NDUFA10 | 4705 || NDUFA11 |
126328 || NDUFA12 | 55967 || NDUFA13 | 51079 || NDUFA2 | 4695 || NDUFA3 | 4696 ||
NDUFA4 | 4697 || NDUFA4L2 | 56901 || NDUFA5 | 4698 || NDUFA6 | 4700 || NDUFA7 |
4701 || NDUFA8 | 4702 || NDUFA9 | 4704 || NDUFAB1 | 4706 || NDUFAF1 | 51103 ||
NDUFAF2 | 91942 || NDUFAF3 | 25915 || NDUFAF4 | 29078 || NDUFAF5 | 79133 ||
NDUFAF6 | 137682 || NDUFAF7 | 55471 || NDUFAF8 | 284184 || NDUFB1 | 4707 ||
NDUFB10 | 4716 || NDUFB11 | 54539 || NDUFB2 | 4708 || NDUFB2-AS1 | 100134713 ||
NDUFB3 | 4709 || NDUFB4 | 4710 || NDUFB5 | 4711 || NDUFB6 | 4712 || NDUFB7 | 4713
|| NDUFB8 | 4714 || NDUFB9 | 4715 || NDUFC1 | 4717 || NDUFC2 | 4718 || NDUFS1 |
4719 || NDUFS2 | 4720 || NDUFS3 | 4722 || NDUFS4 | 4724 || NDUFS5 | 4725 || NDUFS6
| 4726 || NDUFS7 | 374291 || NDUFS8 | 4728 || NDUFV1 | 4723 || NDUFV2 | 4729 ||
NDUFV3 | 4731 || NUBPL | 80224 || OXA1L | 5018 || RFESD | 317671 || SCO1 | 6341 ||
SCO2 | 9997 || SLC25A4 | 291 || SURF1 | 6834 || TACO1 | 51204 || TIMMDC1 | 51300 ||
TMEM126B | 55863 || TRAP1 | 10131 || TTC19 | 54902 || UQCC1 | 55245 || UQCC2 |
84300 || UQCC3 | 790955 || UQCR10 | 29796 || UQCR11 | 10975 || UQCRB | 7381 ||
UQCRC1 | 7384 || UQCRC2 | 7385 || UQCRFS1 | 7386 || UQCRH | 7388 || UQCRHL |
440567 || UQCRQ | 27089 ||

TABLE 25-22
Module: pDC

CLEC4C | 170482 || NRP1 | 8829 || IL3RA | 3563 ||

TABLE 25-23
Module: Plasma Cells

C19orf10 | 56005 || IGH | 3492 || IGHD | 3495 || IGHG1 | 3500 || IGHMBP2 | 3508 ||
IGHV2-5 | 28457 || IGHV3-20 | 28445 || IGHV3-23 | 28442 || IGHV4-28 | 28400 ||
IGHV4-31 | 28396 || IGHV4-34 | 28395 || IGK | 50802 || IGKC | 3514 || IGL | 3535 ||
IGLJ3 | 28831 || IGLL1 | 3543 || IGLV@ | 3546 || IGLV1-40 | 28825 || IGLV1-44 | 28823 ||
IGLV2-14 | 28815 || IGLV2-5 | 28818 || IGLV3-1 | 28809 || IGLV3-19 | 28797 || IGLV3-25
| 28793 || IGLV4-3 | 28786 || IGLV4-60 | 28785 || IGLV5-45 | 28781 || IGLV6-57 | 28778 ||
IGLVI-70 | 28763 || MZB1 | 51237 || PRDM1 | 639 || SDC1 | 6382 || THEMIS2 | 9473 ||
TNFRSF17 | 608 ||

TABLE 25-24
Module: SNOR LOW Up

CEACAM1 | 634 || FCGR1 | 2209 || LGALS1 | 3956 || SNORD24 | 26820 || SNORD44 |
26806 || SNORD47 | 26802 || SNORD80 | 26774 ||

TABLE 25-25
Module: T Cells

CCR3 | 1232 || CD226 | 10666 || CD247 | 919 || CD28 | 940 || CD3D | 915 || CD3E | 916 ||
CD3G | 917 || CD4 | 920 || CD5 | 921 || CD8A | 925 || CD8B | 926 || ETS1 | 2113 || GATA3
| 2625 || GRAP2 | 9402 || LEF1 | 51176 || SH2D1A | 4068 || TRAC | 28755 || TRBC1 |
28639 || TRDC | 28526 ||

TABLE 25-26
Module: T reg

FOXP3 | 50943 || IKZF2 | 22807 ||

TABLE 25-27
Module: TCRA

TRAV10 | 28676 || TRAV1-1 | 28693 || TRAV1-2 | 28692 || TRAV12-1 | 28674 ||
TRAV12-2 | 28673 || TRAV12-3 | 28672 || TRAV13-1 | 28671 || TRAV13-2 | 28670 ||
TRAV14DV4 | 28669 || TRAV16 | 28667 || TRAV17 | 28666 || TRAV18 | 28665 ||
TRAV19 | 28664 || TRAV2 | 28691 || TRAV20 | 28663 || TRAV21 | 28662 || TRAV22 |
28661 || TRAV23DV6 | 28660 || TRAV24 | 28659 || TRAV25 | 28658 || TRAV26-1 |
28657 || TRAV26-2 | 28656 || TRAV27 | 28655 || TRAV29DV5 | 28653 || TRAV3 | 28690
|| TRAV30 | 28652 || TRAV34 | 28648 || TRAV35 | 28647 || TRAV36DV7 | 28646 ||
TRAV38-1 | 28644 || TRAV38-2DV8 | 28643 || TRAV39 | 28642 || TRAV4 | 28689 ||
TRAV40 | 28641 || TRAV41 | 28640 || TRAV5 | 28688 || TRAV7 | 28686 || TRAV8-1 |
28685 || TRAV8-2 | 28684 || TRAV8-3 | 28683 || TRAV8-4 | 28682 || TRAV8-6 | 28680 ||
TRAV8-7 | 28679 || TRAV9-1 | 28678 || TRAV9-2 | 28677 ||

TABLE 25-28
Module: TCRAJ

TRAJ10 | 28745 || TRAJ11 | 28744 || TRAJ12 | 28743 || TRAJ13 | 28742 || TRAJ14 | 28741
|| TRAJ15 | 28740 || TRAJ16 | 28739 || TRAJ17 | 28738 || TRAJ18 | 28737 || TRAJ19 |
28736 || TRAJ20 | 28735 || TRAJ21 | 28734 || TRAJ22 | 28733 || TRAJ23 | 28732 ||
TRAJ24 | 28731 || TRAJ25 | 28730 || TRAJ26 | 28729 || TRAJ27 | 28728 || TRAJ28 | 28727
|| TRAJ29 | 28726 || TRAJ3 | 28752 || TRAJ30 | 28725 || TRAJ31 | 28724 || TRAJ32 |
28723 || TRAJ33 | 28722 || TRAJ34 | 28721 || TRAJ35 | 28720 || TRAJ36 | 28719 ||
TRAJ37 | 28718 || TRAJ38 | 28717 || TRAJ39 | 28716 || TRAJ4 | 28751 || TRAJ40 | 28715
|| TRAJ41 | 28714 || TRAJ42 | 28713 || TRAJ43 | 28712 || TRAJ44 | 28711 || TRAJ45 |
28710 || TRAJ46 | 28709 || TRAJ47 | 28708 || TRAJ48 | 28707 || TRAJ49 | 28706 || TRAJ5
| 28750 || TRAJ50 | 28705 || TRAJ52 | 28703 || TRAJ53 | 28702 || TRAJ54 | 28701 ||
TRAJ56 | 28699 || TRAJ57 | 28698 || TRAJ58 | 28697 || TRAJ59 | 28696 || TRAJ6 | 28749
|| TRAJ61 | 28694 || TRAJ7 | 28748 || TRAJ8 | 28747 || TRAJ9 || 28746 ||

TABLE 25-29
Module: TCRB

TRBC2 | 28638 || TRBJ2-1 | 28629 || TRBJ2-2 | 28628 || TRBJ2-2P | 28627 || TRBJ2-3 |
28626 || TRBJ2-4 | 28625 || TRBJ2-5 | 28624 || TRBJ2-6 | 28623 || TRBJ2-7 | 28622 ||
TRBV1 | 28621 || TRBV10-1 | 28585 || TRBV10-2 | 28584 || TRBV11-1 | 28582 ||
TRBV11-2 | 28581 || TRBV19 | 28568 || TRBV2 | 28620 || TRBV20-1 | 28567 || TRBV21-
1 | 28566 || TRBV23-1 | 28564 || TRBV24-1 | 28563 || TRBV25-1 | 28562 || TRBV27 |
28560 || TRBV28 | 28559 || TRBV3-1 | 28619 || TRBV4-1 | 28617 || TRBV4-2 | 28616 ||
TRBV5-1 | 28614 || TRBV5-3 | 28612 || TRBV5-4 | 28611 || TRBV5-5 | 28610 || TRBV5-6
| 28609 || TRBV5-7 | 28608 || TRBV6-1 | 28606 || TRBV6-4 | 28603 || TRBV6-5 | 28602 ||
TRBV6-6 | 28601 || TRBV6-7 | 28600 || TRBV6-8 | 28599 || TRBV7-1 | 28597 || TRBV7-3
| 28595 || TRBV7-4 | 28594 || TRBV7-5 | 28593 || TRBV7-6 | 28592 || TRBV7-7 | 28591 ||
TRBV9 | 28586 ||

TABLE 25-30
Module: TCRD

TRDC | 28526 || TRDJ1 | 28522 || TRDJ2 | 28521 || TRDJ3 | 28520 || TRDJ4 | 28519 ||
TRDV1 | 28518 || TRDV2 | 28517 || TRDV3 | 28516 ||

TABLE 25-31
Module: TNF Waddel Up

ACLY | 47 || ACSL1 | 2180 || ADGRE2 | 30817 || AK3 | 50808 || AKAP10 | 11216 ||
AMPD3 | 272 || APOL3 | 80833 || ARID3A | 1820 || ARSE | 415 || ASAP1 | 50807 ||
B4GALT5 | 9334 || BCL2A1 | 597 || BHLHE41 | 79365 || BHMT | 635 || BIRC3 | 330 ||
BRCA1 | 672 || CALD1 | 800 || CASP1 | 834 || CASP10 | 843 || CCL15 | 6359 || CCL20 |
6364 || CCL23 | 6368 || CCL3L1 | 6349 || CD37 | 951 || CD38 | 952 || CD83 | 9308 ||
CDKN3 | 1033 || CKB | 1152 || CR2 | 1380 || CTNND2 | 1501 || CXCL1 | 2919 || CXCL2 |
2920 || CXCL3 | 2921 || CXCL8 | 3576 || CYP27B1 | 1594 || DAB2 | 1601 || EBI3 | 10148 ||
EGR1 | 1958 || EGR2 | 1959 || EPB41 | 2035 || EREG | 2069 || ETAA1 | 54465 || F3 | 2152 ||
FABP1 | 2168 || FBXL2 | 25827 || FCER2 | 2208 || FCGR2A | 2212 || FLJ11129 | 54674 ||
FLNA | 2316 || G0S2 | 50486 || GBP1 | 2633 || GCH1 | 2643 || GJB2 | 2706 || GLS | 2744 ||
GMIP | 51291 || GP1BA | 2811 || GRK3 | 157 || HCAR3 | 8843 || HHEX | 3087 || HOMER2
| 9455 || HP | 3240 || ICAM1 | 3383 || IDO1 | 3620 || IFI44 | 10561 || IKBKG | 8517 || IL16 |
3603 || IL18 | 3606 || IL1A | 3552 || IL1B | 3553 || IL1RN | 3557 || IL6 | 3569 || INHBA |
3624 || INSIG1 | 3638 || ITGA6 | 3655 || KITLG | 4254 || KLF1 | 10661 || KMO | 8564 ||
LGALS3BP | 3959 || MAP3K4 | 4216 || MARCKS | 4082 || MGLL | 11343 || MMP19 |
4327 || MN1 | 4330 || MRPS15 | 64960 || MSC | 9242 || MTF1 | 4520 || MX1 | 4599 ||
NAMPT | 10135 || NELL2 | 4753 || NFKB1 | 4790 || NFKB2 | 4791 || NFKBIA | 4792 ||
NFKBIZ | 64332 || NKX3-2 | 579 || NR3C1 | 2908 || OAS3 | 4940 || PATJ | 10207 ||
PDE4DIP | 9659 || PDPN | 10630 || PIAS4 | 51588 || PLAUR | 5329 || PTGES | 9536 ||
PTGS2 | 5743 || RELB | 5971 || RPGR | 6103 || RPS9 | 6203 || SDC4 | 6385 || SERPIND1 |
3053 || SFRP1 | 6422 || SH3BP5 | 9467 || SLAMF1 | 6504 || SLC30A4 | 7782 || SOD2 |
6648 || SPI1 | 6688 || SSPN | 8082 || STAT4 | 6775 || TAF15 | 8148 || TAP2 | 6891 || TBX3 |
6926 || TFF1 | 7031 || TNF | 7124 || TNFAIP2 | 7127 || TNFAIP3 | 7128 || TNFRSF11A |
8792 || TRAF1 | 7185 || TSC22D1 | 8848 || TYROBP | 7305 || UBE2C | 11065 || VEGFA |
7422 || WT1 | 7490 ||

TABLE 25-32
Module: Unfolded Protein

B4GALT3 | 8703 || CALR | 811 || CALU | 813 || CANX | 821 || CDS2 | 8760 || CHST12 |
55501 || CHST2 | 9435 || DERL1 | 79139 || DERL2 | 51009 || DNAJC3 | 5611 || EDEM2 |
55741 || EDEM3 | 80267 || EMC9 | 51016 || ERAP1 | 51752 || ERGIC2 | 51290 || ERO1L |
30001 || EXT1 | 2131 || GALNT2 | 2590 || GOLT1B | 51026 || HERPUD1 | 9709 || HYOU1
| 10525 || IER3IP1 | 51124 || IMPAD1 | 54928 || KDELC1 | 79070 || KDELR2 | 11014 ||
LMAN2 | 10960 | LPGAT1 | 9926 | MAN1A1 | 4121 | MANEA | 79694 | MANF | 7873 ||
NUCB2 | 4925 || PDIA4 | 9601 || PDIA6 | 10130 || PIGK | 10026 || PPIB | 5479 || SEC24D |
9871 || SEC61G | 23480 || SPCS3 | 60559 || SSR1 | 6745 || SSR3 | 6747 || TRAM1 | 23471 ||
TRAM2 | 9697 || UGGT1 | 56886 || XBP1 | 7494 ||

### REFERENCES

(1) Kingsmore K M, Bachali P, Catalina M D, et al. Altered expression of genes controlling metabolism characterizes the tissue response to immune injury in lupus. Sci Rep. 2021; 11(1):14789.
(2) Northcott M, Gearing U J, Nim H T, et al. Glucocorticoid gene signatures in systemic lupus erythematosus and the effects of type I interferon: a cross-sectional and in-vitro study. The Lancet Rheumatology. 2021; 3(5):e357-e370.
(3) Wang G, Durussel J, Shurlock J, et al. Validation of whole-blood transcriptome signature during microdose recombinant human erythropoietin (Rhuepo) administration. BMC Genomics. 2017; 18(8):817.
(4) Dale D C, Fauci A S, Guerry D I V, Wolff S M. Comparison of agents producing a neutrophilic leukocytosis in man. Hydrocortisone, prednisone, endotoxin, and etiocholanolone. J Clin Invest. 1975; 56(4):808-813.
(5) Martin M, Guffroy A, Argemi X, Martin T. [Systemic lupus erythematosus and lymphopenia: Clinical and pathophysiological features]. Rev Med Interme. 2017; 38(9):603-613.
(6) Portolés J, Martin L, Broseta J J, Cases A. Anemia in chronic kidney disease: from pathophysiology and current treatments, to future agents. Front Med. 2021; 8:642296.

While preferred embodiments have been shown and described herein, such embodiments are provided by way of example only. Numerous variations, changes, and substitutions will now occur to those skilled in the art without departing from the scope of the disclosure. It may be understood that various alternatives to the embodiments described herein may be employed in practice. Numerous different combinations of embodiments described herein are possible, and such combinations are considered part of the present disclosure. In addition, all features discussed in connection with any one embodiment herein may be readily adapted for use in other embodiments herein. It is intended that the following claims define the scope of the disclosure and that methods and structures within the scope of these claims and their equivalents be covered thereby.

1. A method for assessing a disease state of a patient, the method comprising: analyzing a data set comprising or derived from gene expression measurement data of at least 2 genes or human orthologs thereof selected from the genes listed in Tables 19-1 to 19-36, Tables 19A-1 to 19A-36, Table 20, Table 21, Table 22, Tables 23-1 to 23-28, Tables 25-1 to 25-32, Tables 26-1 to 26-60, Tables 27-1 to 27-48, and Tables 28-1 to 28-22 in a biological sample from the patient, to classify the disease state of the patient, wherein the disease state of the patient is lupus nephritis.

2. The method of claim 1, wherein the lupus nephritis disease state of the patient is classified as acute lupus nephritis, transitional lupus nephritis, chronic lupus nephritis, or absence of lupus nephritis.

3. The method of claim 1, wherein the data set comprises or is derived from gene expression measurement data of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 450, 500, 550, 600, 650, 700, 750, 850, 900, 950, 1000, 1050, 1100, 1150, 1200, 1250, 1300, 1350, 1400, 1450, 1500, 1550, 1600, 1700, 1800, 1900, or 2000 genes, selected from the genes listed in Tables 19-1 to 19-36, Tables 19A-1 to 19A-36, Table 20, Table 21, Table 22, Tables 23-1 to 23-28, Tables 25-1 to 25-32, Tables 26-1 to 26-60, Tables 27-1 to 27-48, and Tables 28-1 to 28-22 in the biological sample from the patient.

4. The method of claim 1, wherein the genes or human orthologs thereof are selected from the genes listed in: (i) Tables 19-1 to 19-36; (ii) Table 20; (iii) Table 21; (iv) Table 22; (v) Tables 23-1 to 23-28; (vi) Tables 25-1 to 25-32; (vii) Tables 26-1 to 26-60; (viii) Tables 27-1 to 28-48; or (ix) Tables 28-1 to 28-22.

5. (canceled)

6. (canceled)

7. (canceled)

8. (canceled)

9. (canceled)

10. (canceled)

11. (canceled)

12. (canceled)

13. The method of claim 1, wherein the data set comprises or is derived from gene expression measurement data of at least 2 to all, or any value or range there between, genes or human orthologs thereof selected from the genes listed in each of one or more Tables selected from Tables 19-1 to 19-36, Tables 19A-1 to 19A-36, Table 20, Table 21, Table 22, Tables 23-1 to 23-28, Tables 25-1 to 25-32, Tables 26-1 to 26-60, Tables 27-1 to 27-48, and Tables 28-1 to 28-22 in the biological sample from the patient, wherein a different or identical number of genes are selected from the genes listed in each selected table.

14. (canceled)

15. The method of claim 13, wherein the one or more Tables comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 Tables selected from Tables 19-1 to 19-36, Tables 19A-1 to 19A-36, Table 20, Table 21, Table 22, Tables 23-1 to 23-28, Tables 25-1 to 25-32, Tables 26-1 to 26-60, Tables 27-1 to 27-48, or Tables 28-1 to 28-22.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. The method of claim 1, wherein the lupus nephritis disease state of the patient is classified with: (i) an accuracy of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%; (ii) a sensitivity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%; (iii) a specificity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%; (iv) a positive predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%; (v) a negative predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%; or (vi) a Receiver operating characteristic (ROC) curve having an Area-Under-Curve (AUC) of at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

33. (canceled)

34. (canceled)

35. (canceled)

36. (canceled)

37. (canceled)

38. The method of claim 1, wherein the data set is derived from the gene expression measurement data using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, Z-score, log 2 expression analysis, or any combination thereof.

39. (canceled)

40. The method of claim 38, wherein the data set comprises one or more GSVA scores of the patient, wherein the one or more GSVA scores are generated based on one or more Tables selected from Tables 19-1 to 19-36, Tables 19A-1 to 19A-36, Tables 23-1 to 23-28, Tables 25-1 to 25-32, Tables 26-1 to 26-60, Tables 27-1 to 27-48, and Tables 28-1 to 28-22, wherein for each selected Table, at least one GSVA score of the patient is generated based on enrichment of expression of at least 2 genes or human orthologs thereof listed in the selected Table, and wherein the one or more GSVA scores comprise each generated GSVA score.

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. The method of claim 40, wherein independently for each selected Table, the at least one GSVA score of the patient is generated based on enrichment of expression of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295 or all genes selected from the genes listed in the respective Table.

60. The method of claim 1, wherein the analyzing the data set comprises providing the data set as an input to a trained machine-learning model to classify the lupus nephritis disease state of the patient, wherein the trained machine-learning model generates an inference indicative of the lupus nephritis disease state of the patient based at least on the data set.

61. The method of claim 60, wherein the data set comprises the one or more GSVA scores of the patient, and the trained machine-learning model generates the inference based at least on the one or more GSVA scores.

62. The method of claim 60, wherein the method further comprises receiving, as an output of the trained machine-learning model, the inference; and/or electronically outputting a report indicating the lupus nephritis disease state of the patient.

63. The method of claim 60, wherein the machine-learning model is trained using linear regression, logistic regression, Ridge regression, Lasso regression, elastic net (EN) regression, support vector machine (SVM), gradient boosted machine (GBM), k nearest neighbors (kNN), generalized linear model (GLM), naïve Bayes (NB) classifier, neural network, Random Forest (RF), deep learning algorithm, linear discriminant analysis (LDA), decision tree learning (DTREE), adaptive boosting (ADB), Classification and Regression Tree (CART), hierarchical clustering, or any combination thereof.

64. The method of claim 60, wherein the lupus nephritis disease state of the patient is classified based on a lupus nephritis disease risk score generated from the data set and/or from the one or more GSVA scores of the patient.

65. (canceled)

66. The method of claim 40, wherein the patient: (i) is at elevated risk of having lupus; (ii) is suspected of having lupus; (iii) is asymptomatic for lupus: (iv) has lupus: (v) is at elevated risk of having lupus nephritis: (vi) is suspected of having lupus nephritis; (vii) is asymptomatic for lupus nephritis; or (viii) has lupus nephritis.

67. (canceled)

68. (canceled)

69. (canceled)

70. (canceled)

71. (canceled)

72. (canceled)

73. (canceled)

74. The method of claim 1, further comprising identifying, selecting, recommending and/or administering a treatment to the patient based at least in part on the classification of the lupus nephritis disease state of the patient.

75. The method of claim 74, wherein the treatment: (i) is configured to treat lupus nephritis; (ii) is configured to reduce a severity of lupus nephritis; (iii) is configured to reduce a risk of having lupus nephritis: (iv) comprises a pharmaceutical composition.

76. (canceled)

77. (canceled)

78. (canceled)

79. The method of claim 1, wherein the biological sample comprises a kidney biopsy sample, a blood sample, isolated peripheral blood mononuclear cells (PBMCs), or any derivative thereof.

80. A method for validating a mouse model useful for identifying and/or characterizing a human disease, the method comprising:
a) providing a gene set capable of classifying a mouse as having an endotype selected from two or more endotypes of the disease;
b) determining human orthologs of the gene set;
c) classifying a human patient as having an endotype selected from the two or more endotypes of the disease using the human orthologs; and
d) using the human orthologs to classify the mouse model as having an endotype selected from the two or more endotypes of the disease,
wherein the endotype of a validated mouse model classified using the human orthologs corresponds to the human endotype of step (c) identified using the human orthologs.

81. (canceled)
